(19) United States
(12) Patent Application Publication
(10) Pub. No.: US 2026/0183407 A1
(43) Pub. Date: Jul. 2, 2026

(54) DYRK/CLK PROTACS AND USES THEREOF

(71) Applicants: Arizona Board of Regents on Behalf of the University of Arizona, Tucson, AZ (US); RWTH Aachen, Medical Faculty, Aachen (DE)

(72) Inventors: Christopher Hulme, Tucson, AZ (US); Kevin Schofield, Tucson, AZ (US); Walter Becker, Aachen (DE); Gerrit Franz Wilms, Aachen (DE)

(21) Appl. No.: 19/130,769

(22) PCT Filed: Nov. 16, 2023
(86) PCT No.: PCT/US2023/080099

§ 371 Date: May 16, 2025

Related U.S. Application Data
(60) Provisional application No. 63/426,218, filed on Nov. 17, 2022.

(51) Int. Cl. A61K 47/55; A61K 47/54; A61P 25/28; C12N 9/99

(52) U.S. Cl. CPC A61K 47/55; A61K 47/545; A61P 25/28; C12N 9/99

(57) ABSTRACT

The present invention relates to bifunctional compounds, which find utility to degrade and (inhibit) one or more of the following kinases: DYRK1A, DYRK1B, DYRK2, DYRK3, CLKI, CLK2, CLK3, CLK4, and HASPIN. In particular, the present invention is directed to compounds, which contain on one end an E3 ubiquitin ligase binding moiety which binds to an E3 ubiquitin ligase and on the other end a moiety which binds one or more of the following kinases: DYRK1A, DYRK1B, DYRK2, DYRK3, CLK1, CLK2, CLK3, CLK4, and HASPIN, such that the one or more kinases is placed in proximity to the ubiquitin ligase to effect degradation (and inhibition) of the one or more kinases. The bifunctional compounds serve as therapeutics for the treatment of Alzheimer's disease, down syndrome, diabetes, an autoimmune disease, an inflammatory disorder (e.g., airway inflammation, osteoarthritis (e.g., knee related osteoarthritis)), cancer (e.g., glioblastoma, prostate cancer, metastatic breast cancer, metastatic lung cancer, multiple myeloma, secondary metastatic tumors of the brain, colorectal cancer), a viral infection (e.g., SARS-COV-2 infection (e.g., COVID-19)), and other diseases.

### CROSS-REFERENCE TO RELATED APPLICATION

This application claims priority to U.S. provisional patent application Ser. No. 63/426,218, filed Nov. 17, 2022, which is incorporated herein by reference in its entirety.

### STATEMENT REGARDING FEDERALLY SPONSORED RESEARCH OR DEVELOPMENT

This invention was made with government support under Grant No. AG067926 awarded by National Institutes of Health. The government has certain rights in the invention.

### FIELD OF THE INVENTION

The present invention relates to bifunctional compounds, which find utility to degrade and (inhibit) one or more of the following kinases: DYRK1A, DYRK1B, DYRK2, DYRK3, CLK1, CLK2, CLK3, CLK4, and HASPIN. In particular, the present invention is directed to compounds, which contain on one end an E3 ubiquitin ligase binding moiety which binds to an E3 ubiquitin ligase and on the other end a moiety which binds one or more of the following kinases: DYRK1A, DYRK1B, DYRK2, DYRK3, CLK1, CLK2, CLK3, CLK4, and HASPIN, such that the one or more kinases is placed in proximity to the ubiquitin ligase to effect degradation (and inhibition) of the one or more kinases. The bifunctional compounds serve as therapeutics for the treatment of Alzheimer's disease, down syndrome, diabetes, an autoimmune disease, an inflammatory disorder (e.g., airway inflammation, osteoarthritis (e.g., knee related osteoarthritis)), cancer (e.g., glioblastoma, prostate cancer, metastatic breast cancer, metastatic lung cancer, multiple myeloma, secondary metastatic tumors of the brain, colorectal cancer), a viral infection (e.g., SARS-COV-2 infection (e.g., COVID-19)), and other diseases.

### INTRODUCTION

It's estimated that 2 million new cases of cancer will be shortly diagnosed per year with over 600,000 deaths in the US per annum. DYRK and CLK kinase inhibition affords opportunities across a spectrum of malignancies. Moreover, small molecule inhibition of DYRK and CLK kinase may play a role in mitigating disease progression of autoimmune disease and inflammatory disorders, exemplified by osteo-arthritis. DYRK1A has been revealed to play a key role in dementia and down syndrome pathogenesis. With >40 million patients suffering, dementia is currently a leading unmet medical need and costly burden on public health. Seventy percent of these cases have been attributed to Alzheimer's disease (AD), a neurodegenerative pathology whose most evident symptom is a progressive decline in cognitive functions. The underlying treatment of learning and/or memory disorders is a huge and significantly unmet medical need and also includes learning and memory repair after incidents of stroke or significant brain damage.

The present invention addresses these needs.

### SUMMARY OF THE INVENTION

The proteasome (immuno- and constitutive), heat shock factor 1, and mammalian target of rapamycin (mTOR) are essential protein complexes responsible for maintaining growth, division, and survival of cells in eukaryotes and are required for almost all cellular activities. Any impairment of any one or more of the complexes often underlies neurodegenerative diseases, cancer, immune disorders, and the aging process. Targeting these complexes has been clinically proven to be effective against all forms of cancers. RNA interference, kinome-wide screen, and biochemical studies demonstrate that blocking 26S proteasome and heat shock factor 1 phosphorylations triggered by DYRK2 markedly impairs proteostasis and impedes cell proliferation (see, Guo et.al. 2016 Nature Cell Biology; Moreno et.al. 2021 Cell death and differentiation; Banerjee et.al. PNAS 2018; Banerjee et.al. PNAS 2019). Furthermore, inhibition of DYRK3 activity leads to loss of PRAS40 phosphorylation leading to loss of mTOR signaling which reduces cancer cell proliferation. Importantly, loss of DYRK2 and DYRK3 activities significantly inhibited tumor formation in mice (see, eg, Banerjee et.al. 2019 PNAS). Accordingly, small-molecule inhibitors of DYRK kinases, either used alone or in combination with existing chemotherapy and/or proteasome inhibitors, have unique therapeutic potentials in treating human cancers with deregulated growth and proliferation.

Moreover, canonical WNT signaling is a key developmental pathway that has garnered significant interest for therapeutic intervention. The ability to modulate the WNT pathway and thus restore the health of diseased tissues affords possibilities in regenerative therapeutics and oncology. Noteworthy, WNT signaling controls chondrocyte osteoblast and synovial cell functions in osteoarthritis (Tao et. al., Theranostics, 2017, 7, 180-195). Indeed, numerous biological processes and targets related to WNT activation have been reported (Zhan et. al., Oncogene 2017, 36, 1461-1473. Ahmed et. al., Cancers, 2016, 8, 66). Part of this set of targets comprises the serine/threonine kinase CLKs shown to modulate the Wnt pathway by regulating pre-mRNA splicing (Deshmukh et. al., Osteoarthritis Cartilage 2019, 27, 1347-1360, Wang et. al., Nature 2008, 456-470-476). They consist as four isoforms in mammals (CLK1 through CLK4) and belong to the CMGC group of kinases which includes DYRKS, cyclin-dependent kinases (CDKs), GSK3, serine-arginine-rich protein kinases (SRPK) and others. CLK protein over-expression affects splicing site selection of pre-mRNA and as such, several CLK family inhibitors have been reported to play roles in the control mechanisms of mRNA splicing (Bossard et al., Cancer Res., 2020, 80, 5691. Deshmukh et. al., Osteoarthritis Cartilage 2019, 27, 1347-1360). Specifically, two high profile CLK inhibitors in clinical trials are SM08502 (Indication: Colorectal cancer, NCT03355066) and SM04690 (Indication: Osteoarthritis of the knee, Phase 3, NCT03928184).

In addition to the overwhelmingly prominent 8-amyloid hypothesis being evaluated in a multitude of clinical trials through small molecule modulation of γ- and β-secretases and numerous immune-based approaches, aberrant phosphorylation of the tau protein is believed to significantly contribute to the development of AD and thus affords an alternate approach for therapeutic development. Tau is a cytoplasmic protein involved in the stabilization of microtubules under normal conditions. In AD, neuronal tau has been found to be excessively phosphorylated, with subsequent generation of aggregates of phosphorylated tau protein, known as “neurofibrillary tangles” (NFTs). NFTs and amyloid plaques are considered the most common hallmarks of AD and are correlated with neurofibrillary degeneration, neuronal death, and dementia.

Interestingly, several protein kinases have been implicated in neuronal development and their overexpression and aberrant activation have been shown to play a significant role in the development of AD via tau phosphorylation. Dual specificity tyrosine phosphorylation regulated kinase-1A (DYRK1A) is important in neuronal development and plays a variety of functional roles within the adult central nervous system. The DYRK1A gene is located within the Down syndrome critical region (DSCR) on human chromosome 21 and current research suggests that overexpression of DYRK1A may be a significant factor leading to cognitive deficits in people with Alzheimer's disease (AD) and Down syndrome (DS).

The selective degradation of target proteins using small molecules is a new approach to the treatment of various diseases. Proteolysis Targeting Chimeric molecules (Protacs) are bifunctional molecules which can simultaneously bind a target protein and an E3 ubiquitin ligase thereby bringing the ligase and target in close proximity These bifunctional molecules allow the efficient ubiquitin transfer from the ligase complex to the target protein which is subsequently recognized by the proteasome and degraded. This degradation of the target protein provides treatment of diseases or conditions modulated through the target protein by effectively lowering the level of said target protein in the cells of the patient. An advantage of Protacs is that a broad range of pharmacological activities is possible, consistent with the degradation/inhibition of targeted proteins from virtually any class or family.

E3 ubiquitin ligases (of which hundreds are known in humans) confer substrate specificity for ubiquitination and therefore are more attractive therapeutic targets than general proteasome inhibitors due to their specificity for certain protein substrates. The development of ligands for E3 ligases has proven challenging.

Protacs employed to target proteins to the E3 ligase IAP (Inhibitors of Apoptosis) through the ligand bestatin have been proposed with limited success, see for example Ohoka et al, Cell Death and Disease, 2014,5,e1513. Unfortunately bestatin is a non-specific ligand with multiple activities. IAP inhibitors are known which can be of use in their own right as antitumour agents (see L. Bai et al. Pharmacology & Therapeutics 144 (2014) 82-95). Apoptosis is one form of programmed cell-death and is a normal cellular process used by multi-cellular organisms to eliminate damaged or unwanted cells. Apoptosis is a tightly regulated process and faulty regulation of apoptosis is implicated in many human diseases, including cancer, autoimmune diseases, inflammation, and neurogenesis (see, Lowe S. W and Lin 2000 Carcinogenesis 21 (3), 485-495, Nicholson D. W. 2000, Nature 407 (6805) 810-816, Reed J. C. 2002 Nat Rev Drug Discovery 1 (2) 111-121).

Selective IAP inhibitors are known which describe IAP inhibitors designed for treating disorders associated with apoptosis, particularly cancer (see, WO 2014031487; WO 2014047024; WO 2014055461; WO 2008128171; WO2008/016893; WO 2014/060768; WO2014/060767; WO15092420).

The present inventors have identified kinase targets which are capable of being degraded by Protacs comprising IAP inhibitors targeting the E3 ligase IAP, in particular the targets DYRK1A, DYRK1B, DYRK2, DYRK3, CLK1, CLK2, CLK3, CLK4, HASPIN, and WNT signaling.

Indeed, experiments conducted during the course of developing embodiments for the present invention designed, synthesized and biologically evaluated various Protac compounds capable of inhibiting one or more of DYRK1A, DYRK1B, DYRK2, DYRK3, and CLK1, CLK2, CLK3, CLK4, and HASPIN.

The DYRK/CLK Protac compounds described herein can also be considered as potential therapeutics for the treatment of developmental diseases such as Down syndrome, and neurodegenerative diseases such as Parkinson's disease, and Huntington's disease. Moreover, the DYRK/CLK Protac compounds of the present invention have been also implicated as potential therapeutics for the treatment of glioblastomas and further potential utility is highlighted in the oncology arena (see, e.g., Ionescu et al., Mini-reviews in Medicinal Chemistry, 2012, 12, 1315-1329). Moreover, the DYRK/CLK Protac compounds of the present invention have been also implicated as potential therapeutics for the treatment of viral infections (e.g., SARS-COV-2 infection (e.g., COVID-19)), and other diseases).

These novel DYRK/CLK Protac compounds may also have utility as general cognitive enhancers, given the published findings that DYRK1A can phosphorylate sirtuin 1, a key regulator of learning and memory (see, e.g., Michan et al., J. Neurosci. 2010, 30 (29), 9695-9707; Guo et al., J Biol. Chem. 2010, 285 (17), 13223-13232). Moreover, the effectiveness of small molecule inhibition of DYRK1A in mitigating both insoluble tau aggregates and amyloid plaques has been demonstrated (see, e.g., Branca et al., Aging Cell, 2017, 16 (5), 1146-1154). The mechanistic rational for this was detailed previously (Smith et al., ACS Chem. Neuroscience, 2012, 3 (11), 857-872). These novel DYRK/CLK Protac compounds may also have further utility as results identify DYRK1A as a physiologically relevant regulator of Treg cell differentiation and suggest a broader role for other DYRK family members in immune homeostasis. As such, new roles may be found in autoimmune diseases such as inflammatory bowel disease and type 1 diabetes (see, e.g., Khor B, et al., eLife 2015; 4: e05920).

Accordingly, the present invention relates to bifunctional compounds, which find utility to degrade and (inhibit) one or more of the following kinases: DYRK1A, DYRK1B, DYRK2, DYRK3, CLK1, CLK2, CLK3, CLK4, and HASPIN. In particular, the present invention is directed to compounds, which contain on one end an E3 ubiquitin ligase binding moiety which binds to an E3 ubiquitin ligase and on the other end a moiety which binds one or more of the following kinases: DYRK1A, DYRK1B, DYRK2, DYRK3, CLK1, CLK2, CLK3, CLK4, and HASPIN, such that the one or more kinases is placed in proximity to the ubiquitin ligase to effect degradation (and inhibition) of the one or more kinases. The bifunctional compounds serve as therapeutics for the treatment of Alzheimer's disease, down syndrome, diabetes, an autoimmune disease, an inflammatory disorder (e.g., airway inflammation, osteoarthritis (e.g., knee related osteoarthritis)), cancer (e.g., glioblastoma, prostate cancer, metastatic breast cancer, metastatic lung cancer, multiple myeloma, secondary metastatic tumors of the brain, colorectal cancer), a viral infection (e.g., SARS-COV-2 infection (e.g., COVID-19)), and other diseases.

The present disclosure describes compounds, including compositions comprising the same, which function to recruit endogenous proteins to an E3 ubiquitin ligase enzyme (e.g., Von Hippel-Lindau (VHL) E3 ubiquitin ligase

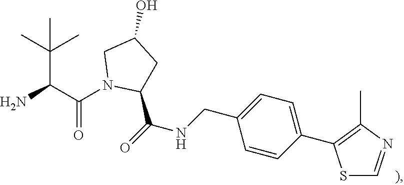 [SMILES: Cc1ncsc1-c1ccc(CNC(=O)[C@@H]2C[C@@H](O)CN2C(=O)[C@@H](N)C(C)(C)C)cc1]

cereblon [symbol] 2-(2,6-dioxopiperidin-3-yl)-5-fluoroisoindoline-1,3-dione [symbol] 3-(1-oxoisoindolin-2-yl)piperidine-2,6-dione [symbol] 2-(1-methyl-2,6-dioxopiperidin-3-yl)isoindoline-1,3-dione [symbol] 3-phenylpiperidine-2,6-dione [symbol] IAP (XIAP), and MDM2) for ubiquitination and subsequent degradation, and methods of using the same. In particular, the present disclosure provides bifunctional or proteolysis targeting chimeric (PROTAC) compounds, which find utility as modulators of targeted ubiquitination and degradation of one or more of DYRK1A, DYRK1B, DYRK2, DYRK3, CLK1, CLK2, CLK3, CLK4, and HASPIN.

In certain embodiments, the present invention provides compounds which function to recruit endogenous proteins (one or more of DYRK1A, DYRK1B, DYRK2, DYRK3, CLK1, CLK2, CLK3, CLK4, and HASPIN) to E3 Ubiquitin Ligase for ubiquitination and degradation.

In certain embodiments, the compounds have the following general structure: Target Binding Moiety (TBM)-L-ULM, ULM-L-TBM, or TBM-ULM.

In some embodiments, the TBM is a small molecule inhibitor of one or more of DYRK1A, DYRK1B, DYRK2, DYRK3, CLK1, CLK2, CLK3, CLK4, and HASPIN, ULM is an E3 ligase binding moiety (e.g., a VHL binding moiety, a cereblon binding moiety, a XIAP binding moiety, or a MDM2 binding moiety), and L is a bond or a linker moiety which links the TBM and ULM.

In certain embodiments, the small molecule inhibitor of one or more of DYRK1A, DYRK1B, DYRK2, DYRK3, CLK1, CLK2, CLK3, CLK4, and HASPIN is selected from 4-(3-(3-fluoro-4-(4-methylpiperazin-1-yl)phenyl)-2-methyl-3H-imidazo[4,5-b]pyridin-5-yl)pyridin-2-amine

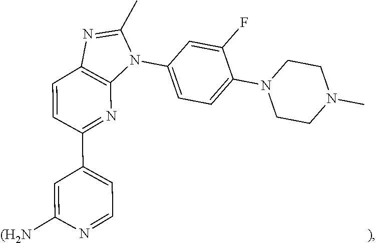

4-(3-(3-methoxy-4-(4-methylpiperazin-1-yl)phenyl)-2-methyl-3H-imidazo[4,5-b]pyridin-5-yl)pyridin-2-amine

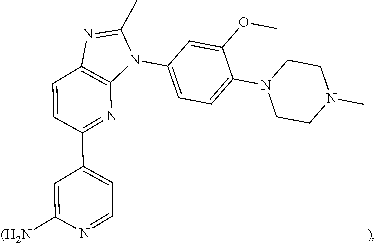

4-(3-(3,5-difluoro-4-(4-(1-methylazetidin-3-yl)piperazin-1-yl)phenyl)-2-methyl-3H-imidazo[4,5-b]pyridin-5-yl)pyridin-2-amine

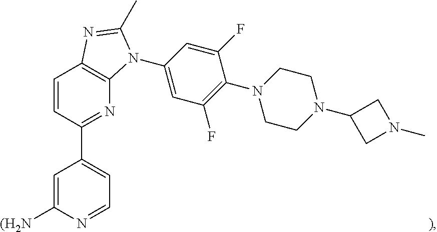

4-(3-(3,5-difluoro-4-(6-methyl-2,6-diazaspiro[3.3]heptan-2-yl)phenyl)-2-methyl-3H-imidazo[4,5-b]pyridin-5-yl)pyridin-2-amine

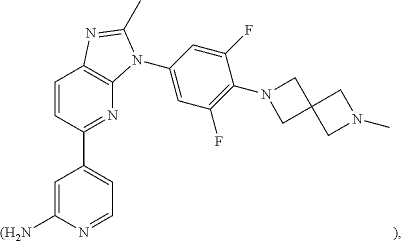

4-(3-(3,5-difluoro-4-((3S,5R)-3,4,5-trimethylpiperazin-1-yl)phenyl)-2-methyl-3H-imidazo[4,5-b]pyridin-5-yl)pyridin-2-amine

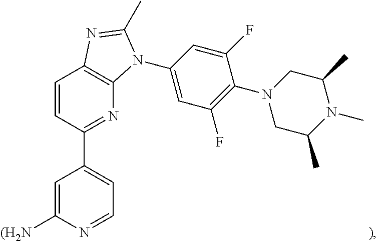

4-(3-(3,5-difluoro-4-(4-(1-methylpiperidin-4-yl)piperazin-1-yl)phenyl)-2-methyl-3H-imidazo[4,5-b]pyridin-5-yl)pyridin-2-amine

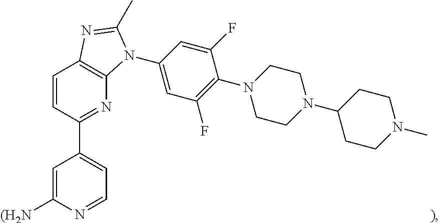

4-(3-(3-fluoro-4-((1S,4S)-5-methyl-2,5-diazabicyclo[2.2.1]heptan-2-yl)phenyl)-2-methyl-3H-imidazo[4,5-b]pyridin-5-yl)pyridin-2-amine

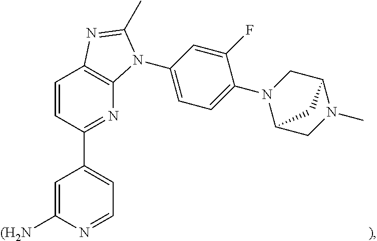

4-(3-(3-fluoro-4-(3-methyl-3,8-diazabicyclo[3.2.1]octan-8-yl)phenyl)-2-methyl-3H-imidazo[4,5-b]pyridin-5-yl)pyridin-2-amine

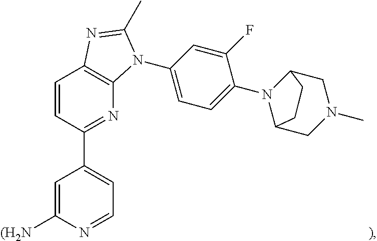

4-(3-(3-fluoro-4-((3aR,6aS)-5-methylhexahydropyrrolo[3,4-c]pyrrol-2 (1H)-yl)phenyl)-2-methyl-3H-imidazo[4,5-b]pyridin-5-yl)pyridin-2-amine

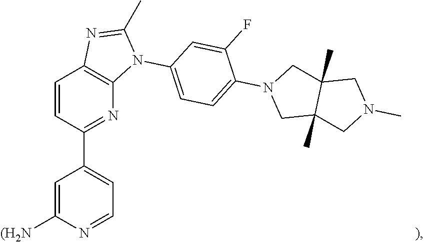

4-(3-(3,5-difluoro-4-(4-methylpiperazin-1-yl)phenyl)-2-methyl-3H-imidazo[4,5-b]pyridin-5-yl)pyridin-2-amine

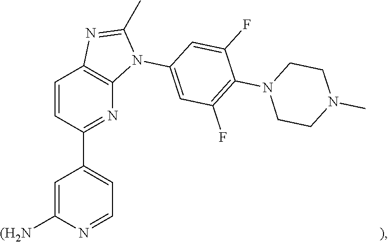

4-(2-methyl-3-(4-(4-methylpiperazin-1-yl)-3-(trifluoromethyl)phenyl)-3H-imidazo[4,5-b]pyridin-5-yl)pyridin-2-amine

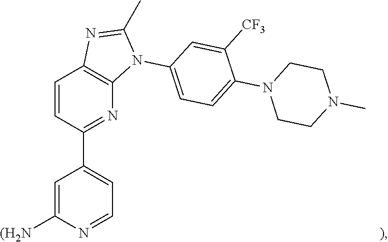

4-(2-methyl-3-(4-((4-methylpiperazin-1-yl)methyl)phenyl)-3H-imidazo[4,5-b]pyridin-5-yl)pyridin-2-amine

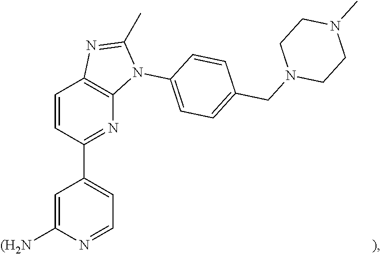

and 4-(3-(3,5-dichloro-4-(4-methylpiperazin-1-yl)phenyl)-2-methyl-3H-imidazo[4,5-b]pyridin-5-yl)pyridin-2-amine

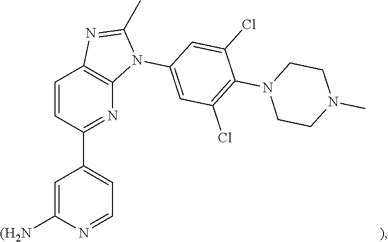

In some embodiments, the TBM covalently couples with the linker at any location within the TBM. In some embodiments related to the small molecule inhibitors of one or more of DYRK1A, DYRK1B, DYRK2, DYRK3, CLK1, CLK2, CLK3, CLK4, and HASPIN, the TBM covalently couples with the linker at the following “ [symbol] ” location:

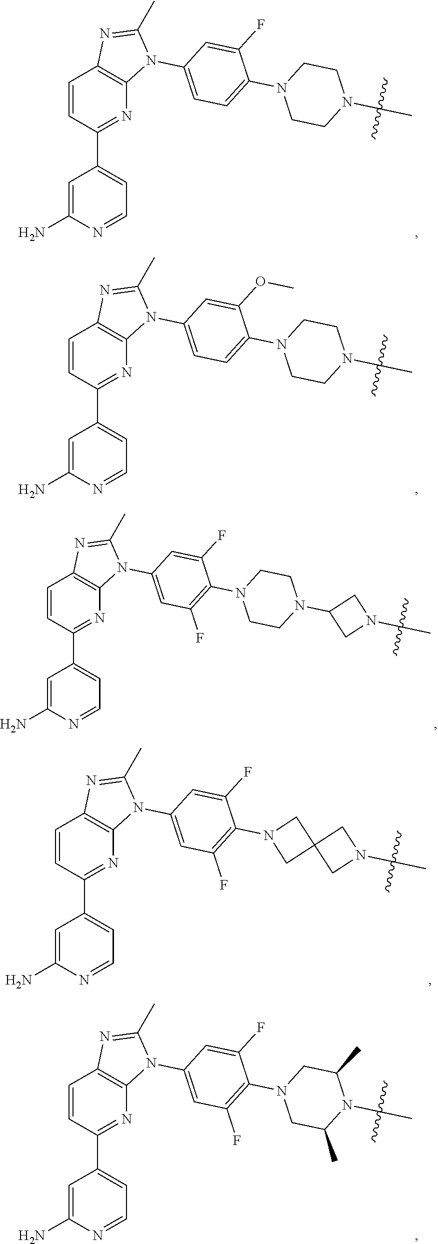

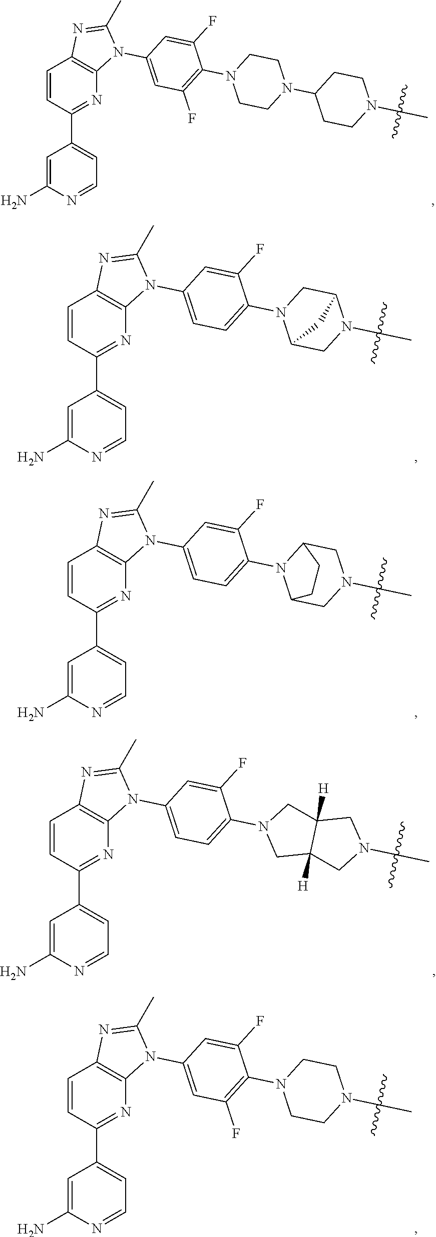

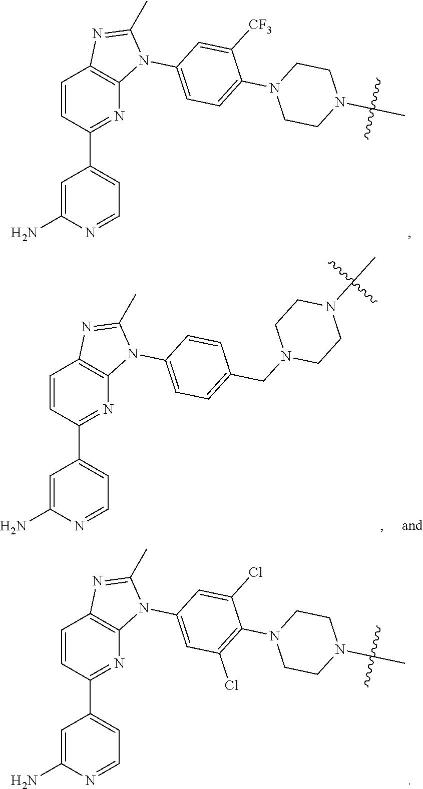 [SMILES: *N1CCN(c2ccc(-n3c(C)nc4ccc(-c5ccnc(N)c5)nc43)cc2C(F)(F)F)CC1 ; *N1CCN(Cc2ccc(-n3c(C)nc4ccc(-c5ccnc(N)c5)nc43)cc2)CC1 ; *N1CCN(c2c(Cl)cc(-n3c(C)nc4ccc(-c5ccnc(N)c5)nc43)cc2Cl)CC1]

In some embodiments, the ULM covalently couples with the linker at any location within the ULM. For example, in some embodiments, the ULM covalently couples with the linker at the following [symbol] location: VHL

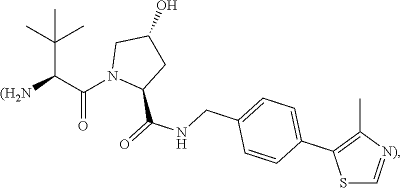

cereblon

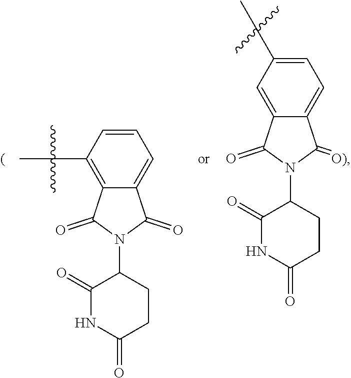

2-(2,6-dioxopiperidin-3-yl)-5-fluoroisoindoline-1,3-dione

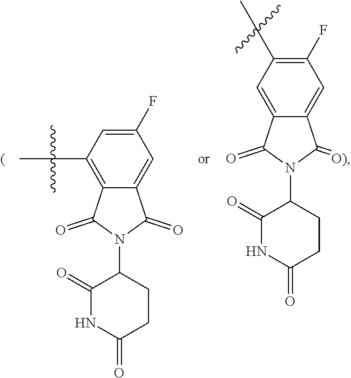

3-(1-oxoisoindolin-2-yl)piperidine-2,6-dione

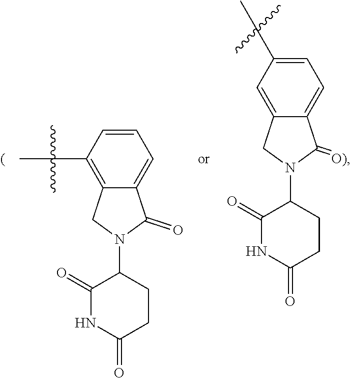

2-(1-methyl-2,6-dioxopiperidin-3-yl)isoindoline-1,3-dione

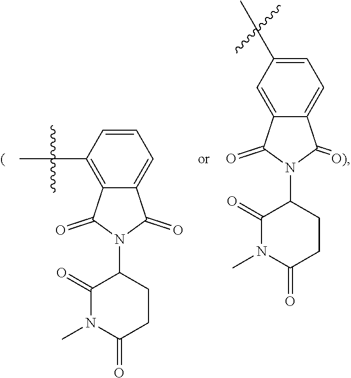

and 3-phenylpiperidine-2,6-dione

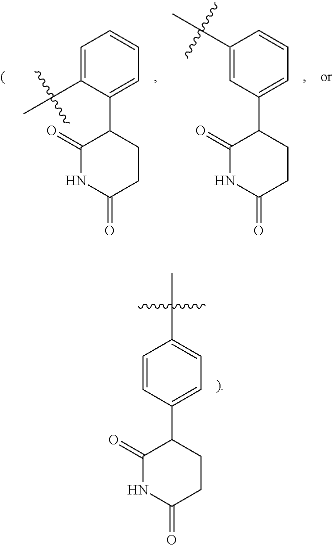 [SMILES: *c1ccccc1C1CCC(=O)NC1=O ; *c1cccc(C2CCC(=O)NC2=O)c1 ; *c1ccc(C2CCC(=O)NC2=O)cc1]

As such, in certain embodiments, the description provides compounds having one of the following general structures: TBM-L-VHL binding moiety, TBM-L-Cereblon binding moiety, TBM-L-XIAP binding moiety, and TBM-L-MDM2 binding moiety.

In certain embodiments, the linker is a bond or a chemical linker that covalently couples the TBM and the ULM.

In some embodiments, the linker is represented by:

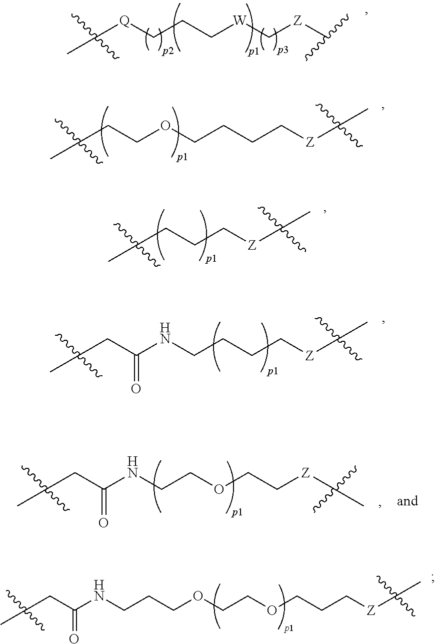

or an enantiomer, diastereomer, or stereoisomer thereof, wherein p1 is an integer selected from 0 to 12; p2 is an integer selected from 0 to 12; p3 is an integer selected from 1 to 6; each W is independently absent, CH₂, O, S, NH, or NR⁸; Z is absent, CH₂, O, NH, or NR⁸; each R⁸ is independently C₁-C₃ alkyl; and Q is absent or CH₂C(O)NH.

In some embodiments, the linker is selected from:

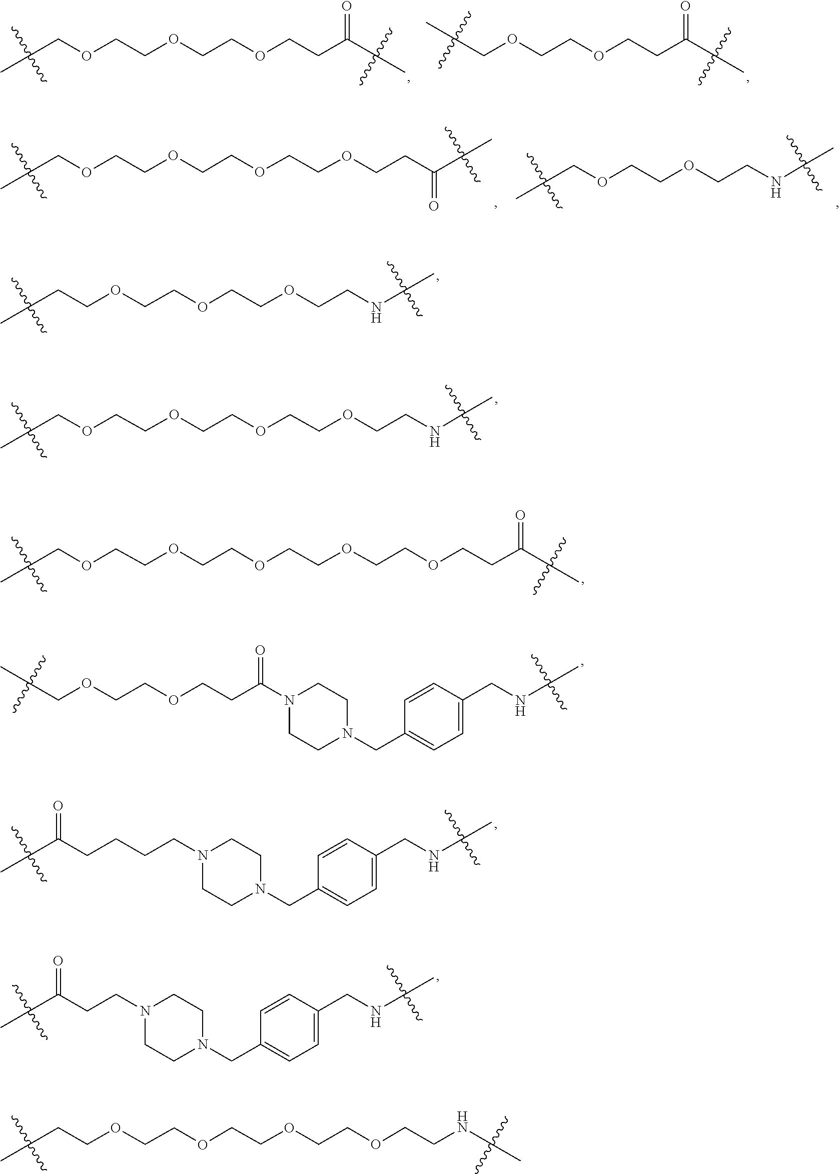

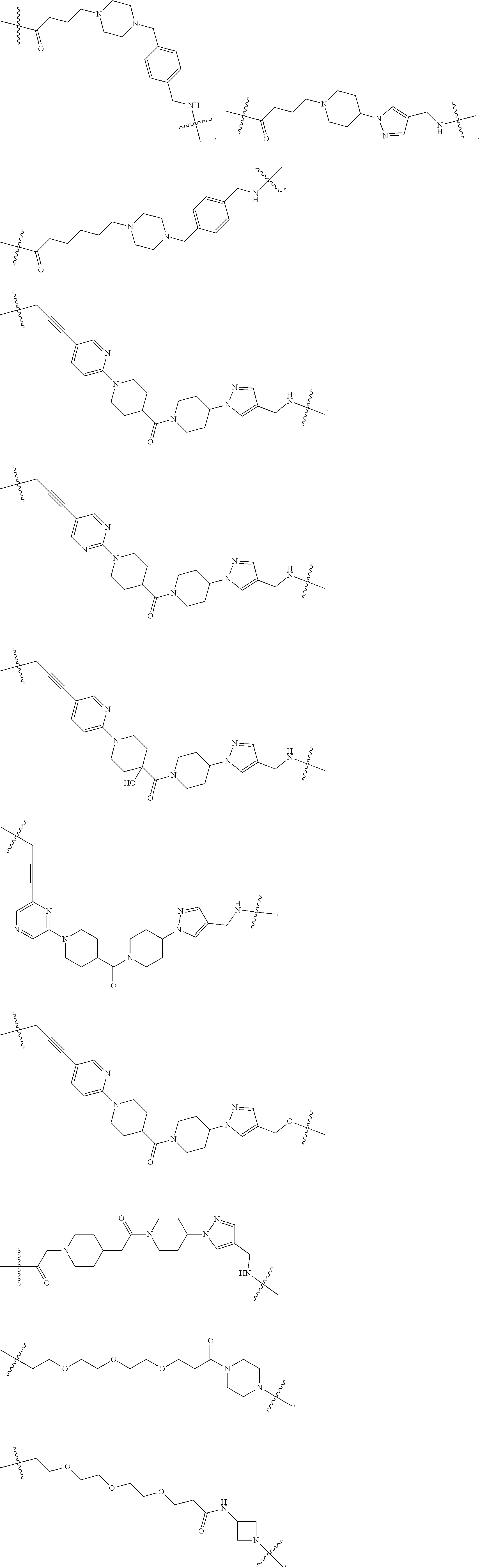

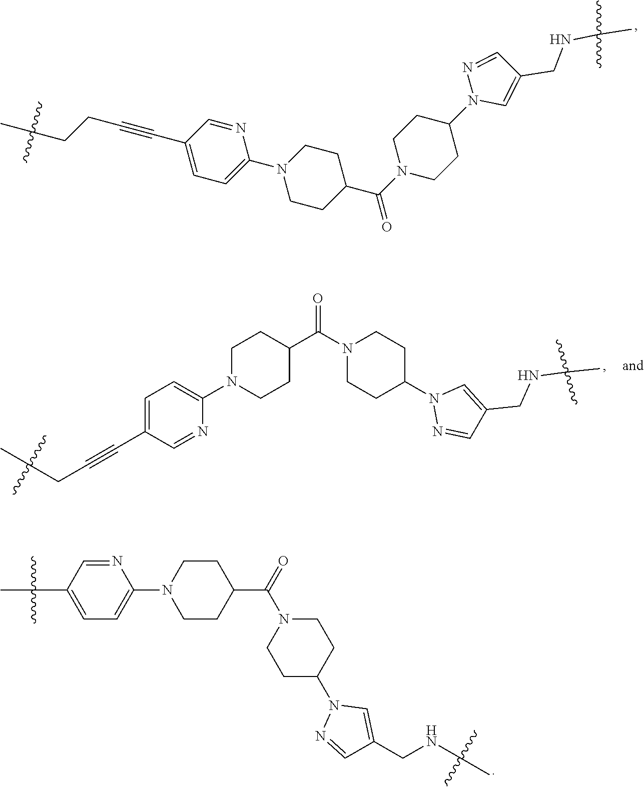 [SMILES: *CCC#Cc1ccc(N2CCC(C(=O)N3CCC(n4cc(CN*)cn4)CC3)CC2)nc1 ; *CC#Cc1ccc(N2CCC(C(=O)N3CCC(n4cc(CN*)cn4)CC3)CC2)nc1 ; *NCc1cnn(C2CCN(C(=O)C3CCN(c4ccc(*)cn4)CC3)CC2)c1]

In some embodiments, TBM-L-ULM is represented by TBM-A-B-modified cereblon, wherein A is represented by

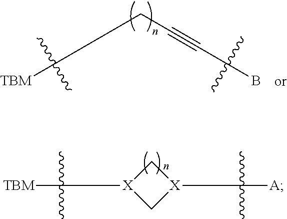

wherein B is represented by

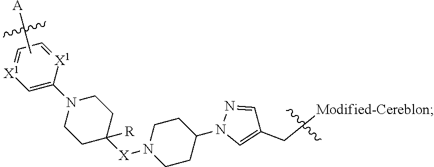

wherein modified-cereblon is selected from:

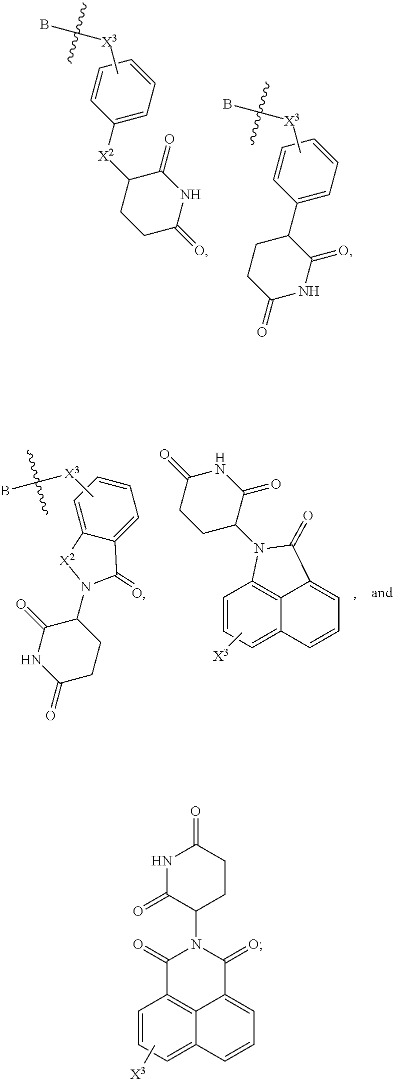

wherein n is 0, 1 or 2; wherein R is OH or H; wherein X is CH₂ or CO; wherein each X¹ is either N or CH; wherein X² is CO, CH₂, or NH; wherein X³ is NH or O.

It will be understood that the general structures are exemplary and the respective moieties can be arranged spatially in any desired order or configuration, e.g., ULM-L-TBM, TBM-L-ULM, TBM-ULM, ULM-TBM respectively.

In certain additional embodiments, the compounds comprise a plurality of E3 ligase binding moieties and/or a plurality of TBMs.

In certain embodiments, the description provides a bifunctional compound having a structure as described herein, a salt, a polymorph, and a prodrug thereof.

In certain embodiments, the bifunctional compound is selected from:

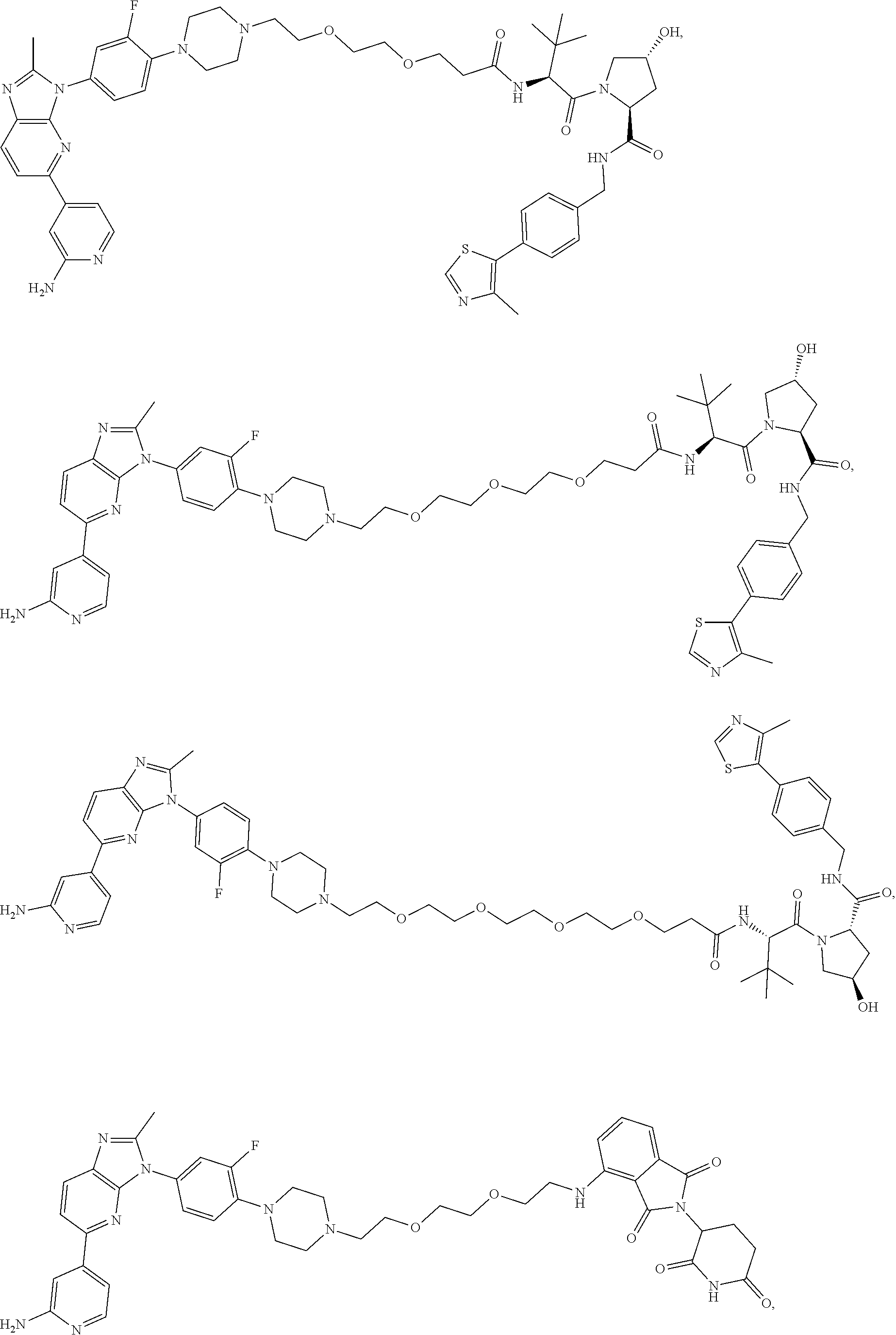

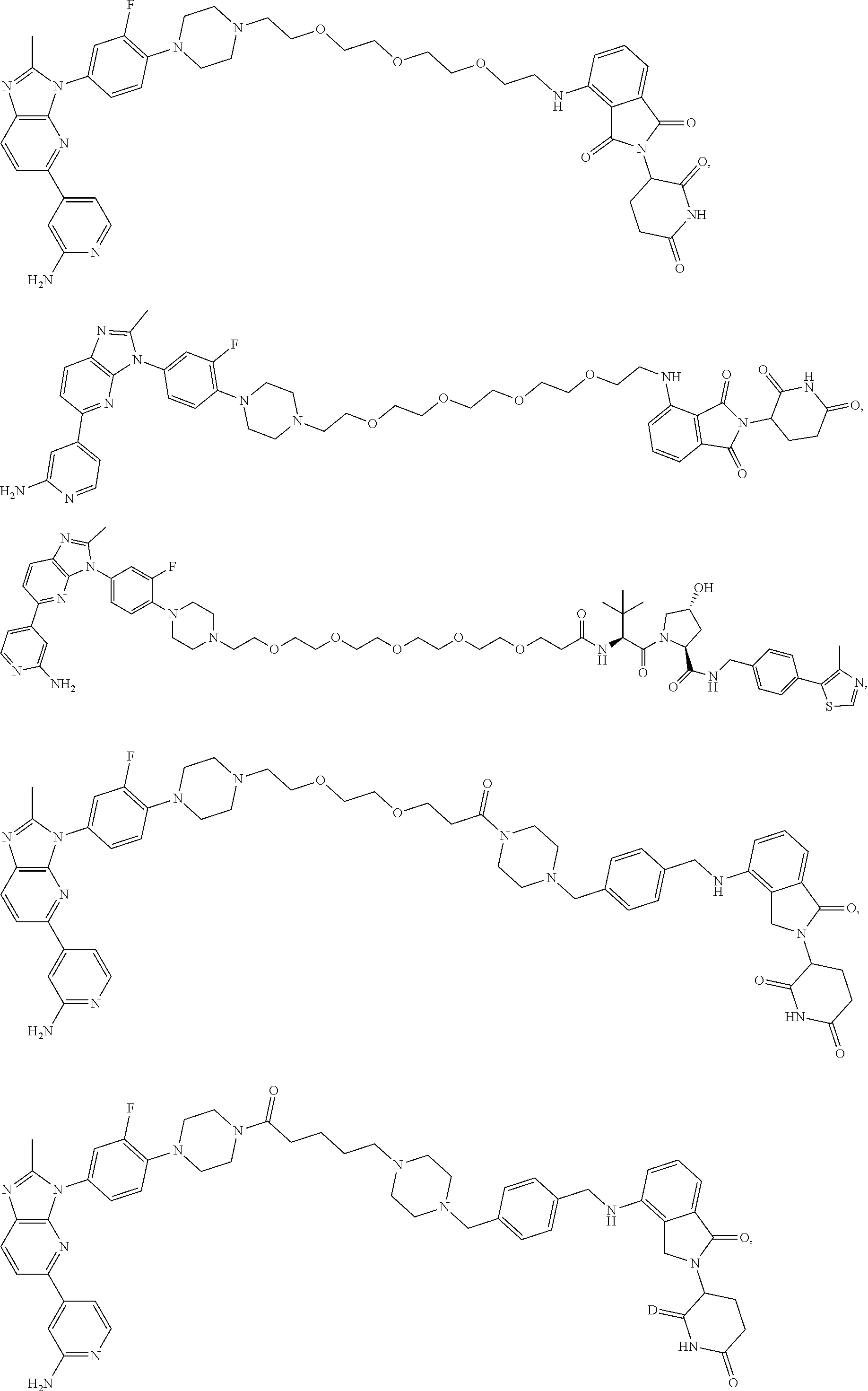

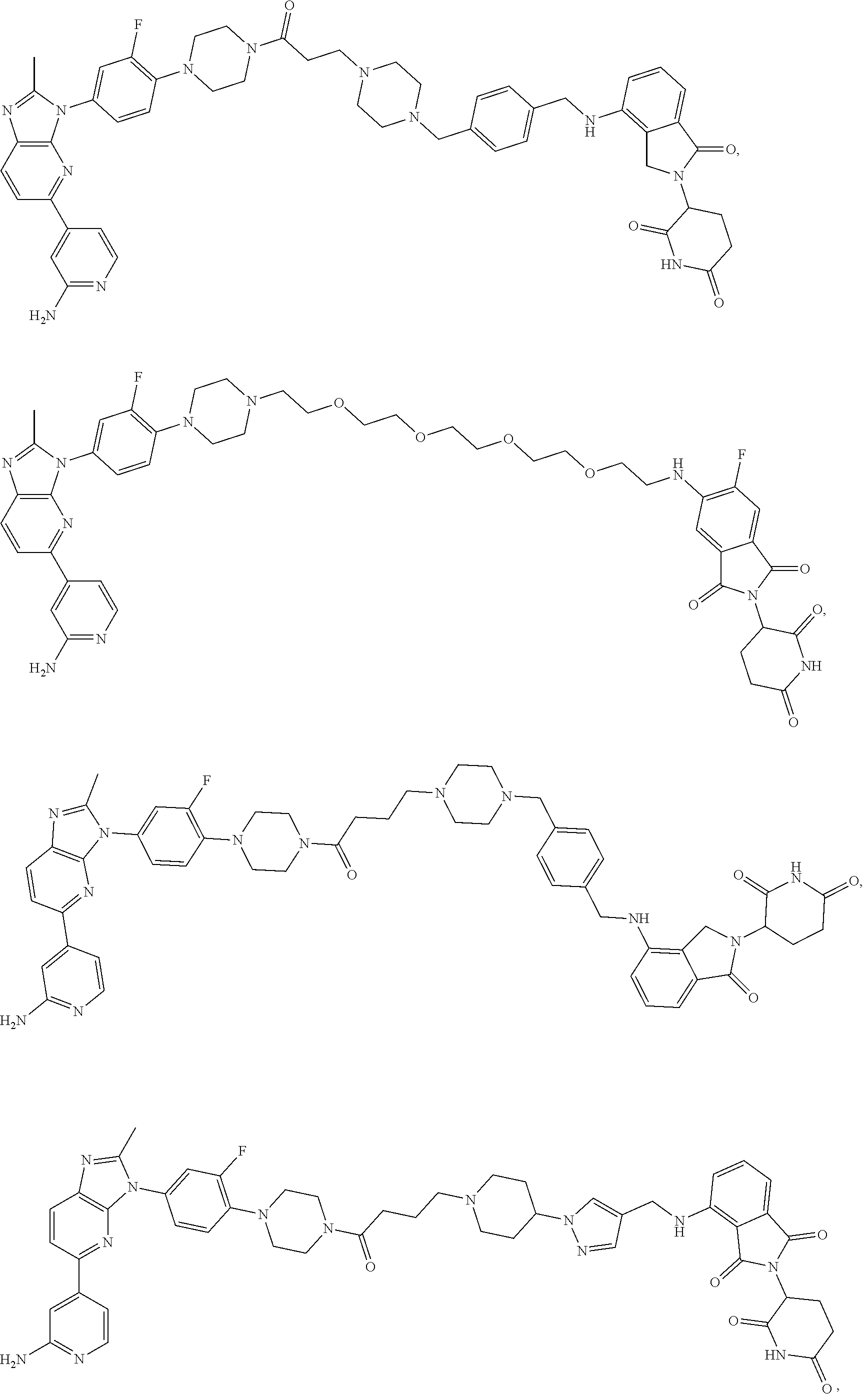 [SMILES: Cc1nc2ccc(-c3ccnc(N)c3)nc2n1-c1ccc(N2CCN(C(=O)CCN3CCN(Cc4ccc(CNc5cccc6c5CN(C5CCC(=O)NC5=O)C6=O)cc4)CC3)CC2)c(F)c1 ; Cc1nc2ccc(-c3ccnc(N)c3)nc2n1-c1ccc(N2CCN(CCOCCOCCOCCOCCNc3cc4c(cc3F)C(=O)N(C3CCC(=O)NC3=O)C4=O)CC2)c(F)c1 ; Cc1nc2ccc(-c3ccnc(N)c3)nc2n1-c1ccc(N2CCN(C(=O)CCCN3CCN(Cc4ccc(CNc5cccc6c5CN(C5CCC(=O)NC5=O)C6=O)cc4)CC3)CC2)c(F)c1 ; Cc1nc2ccc(-c3ccnc(N)c3)nc2n1-c1ccc(N2CCN(C(=O)CCCN3CCC(n4cc(CNc5cccc6c5C(=O)N(C5CCC(=O)NC5=O)C6=O)cn4)CC3)CC2)c(F)c1]

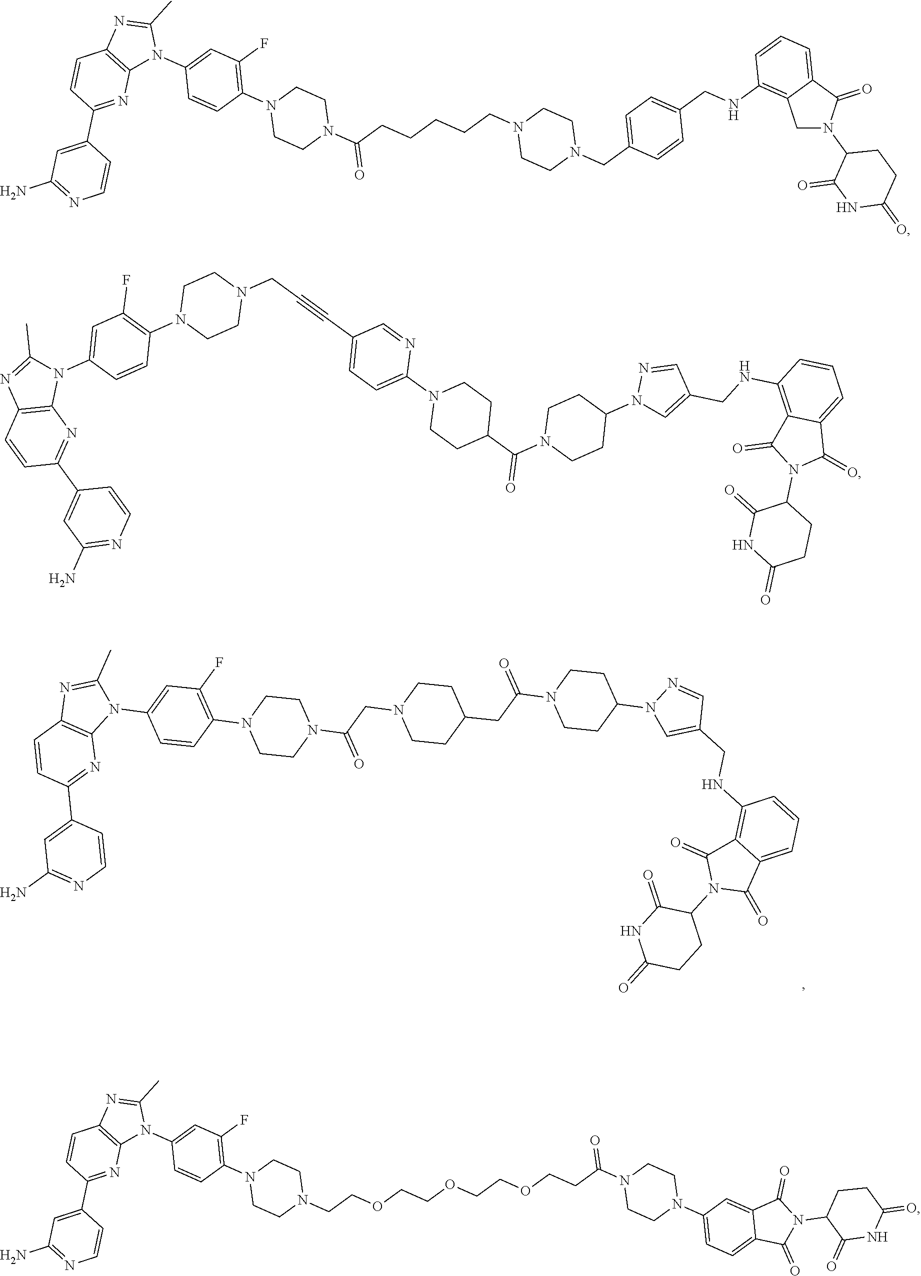

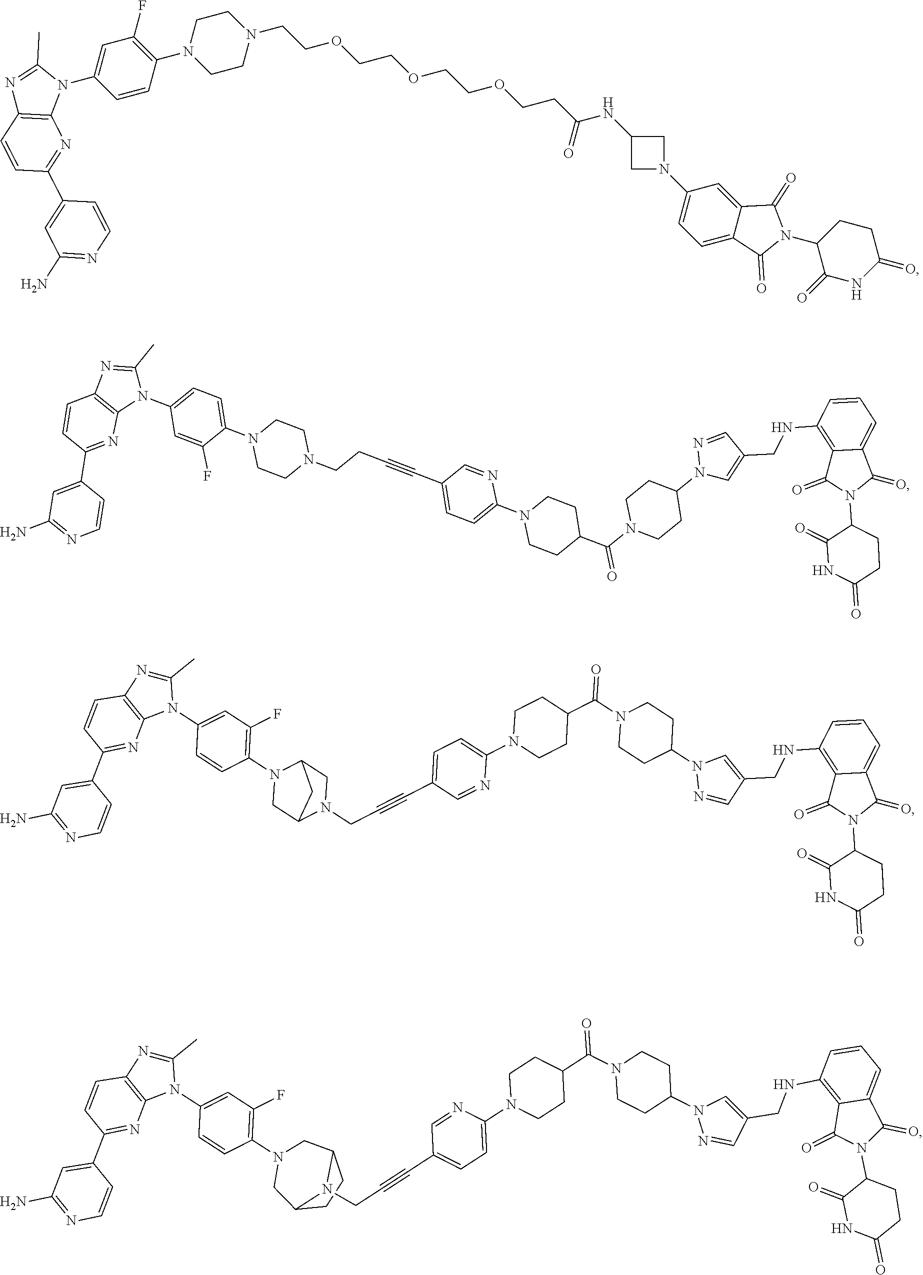

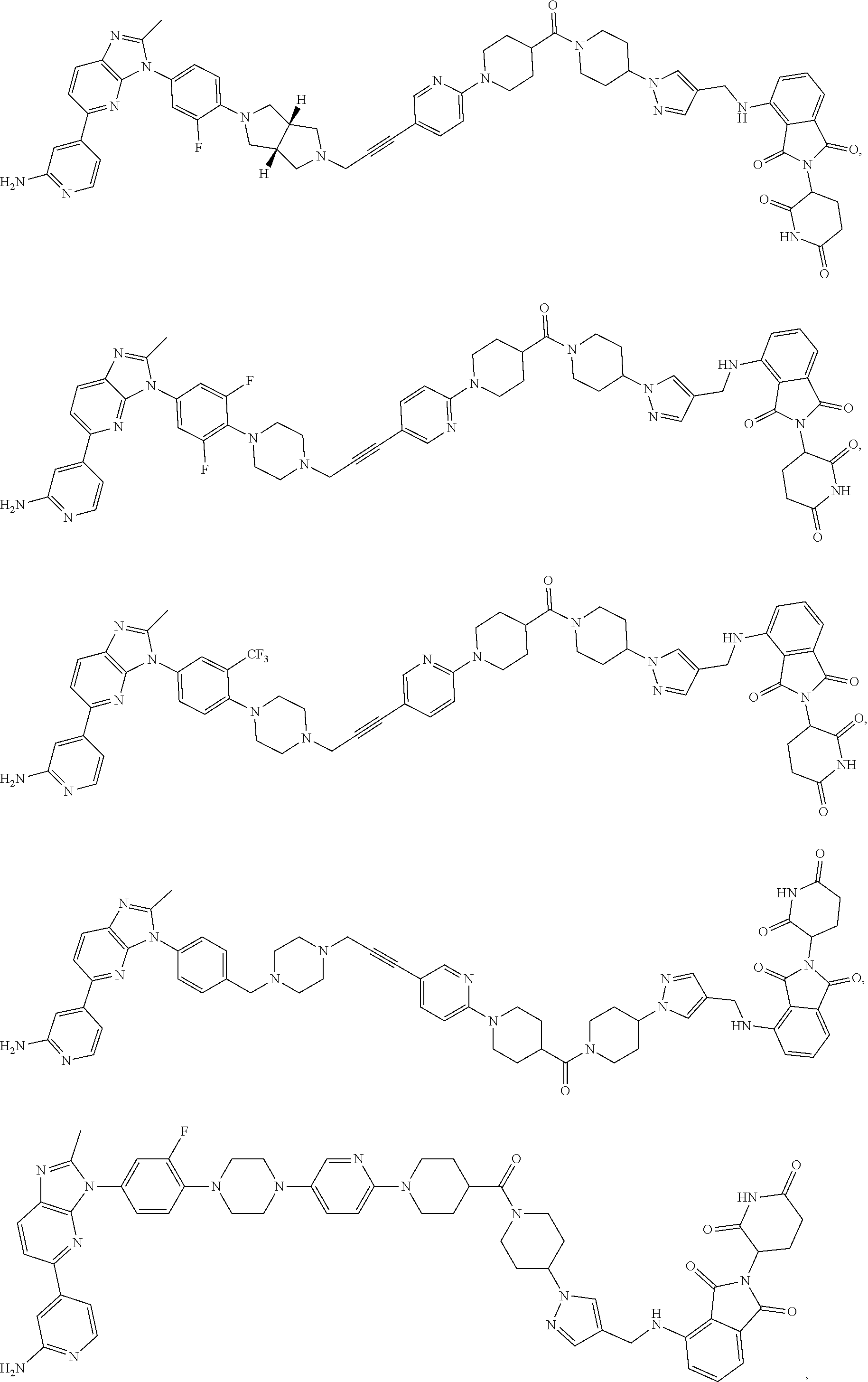

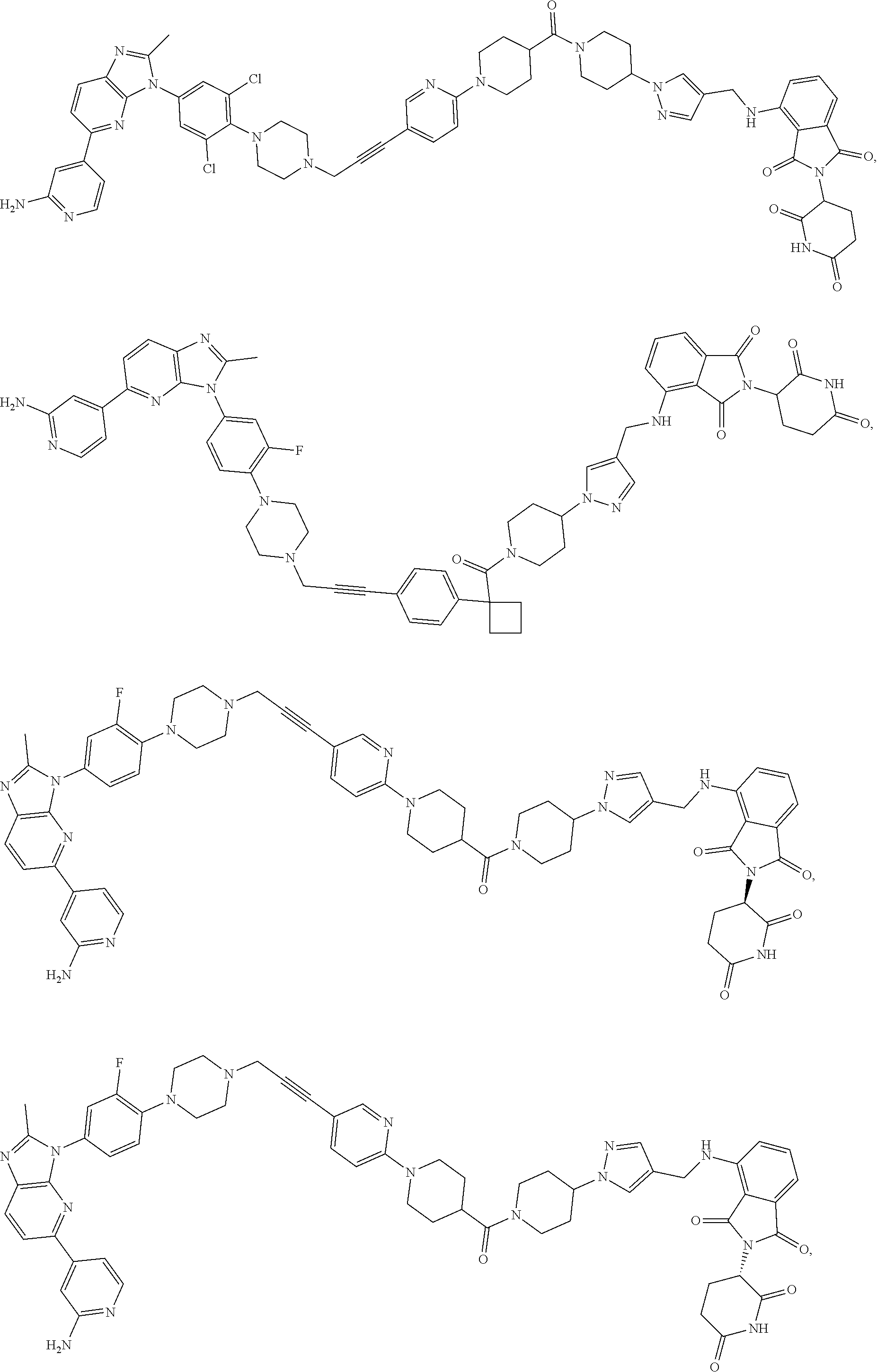

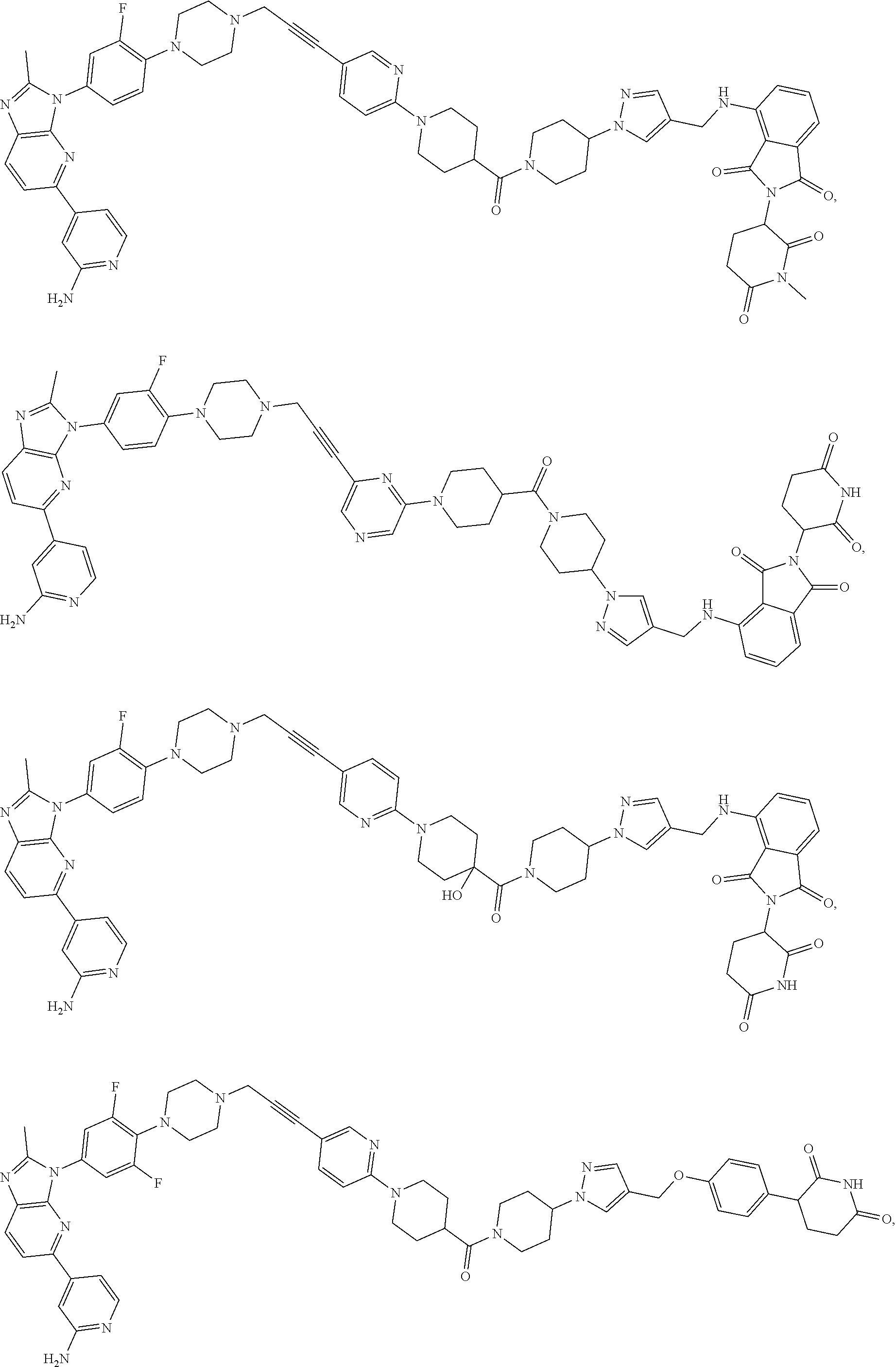

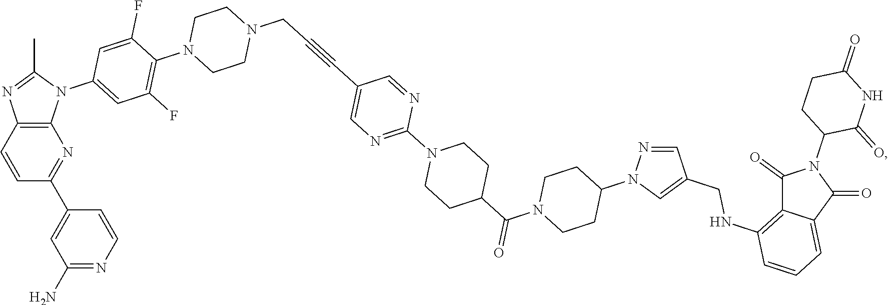 [SMILES: Cc1nc2ccc(-c3ccnc(N)c3)nc2n1-c1cc(F)c(N2CCN(CC#Cc3cnc(N4CCC(C(=O)N5CCC(n6cc(CNc7cccc8c7C(=O)N(C7CCC(=O)NC7=O)C8=O)cn6)CC5)CC4)nc3)CC2)c(F)c1]

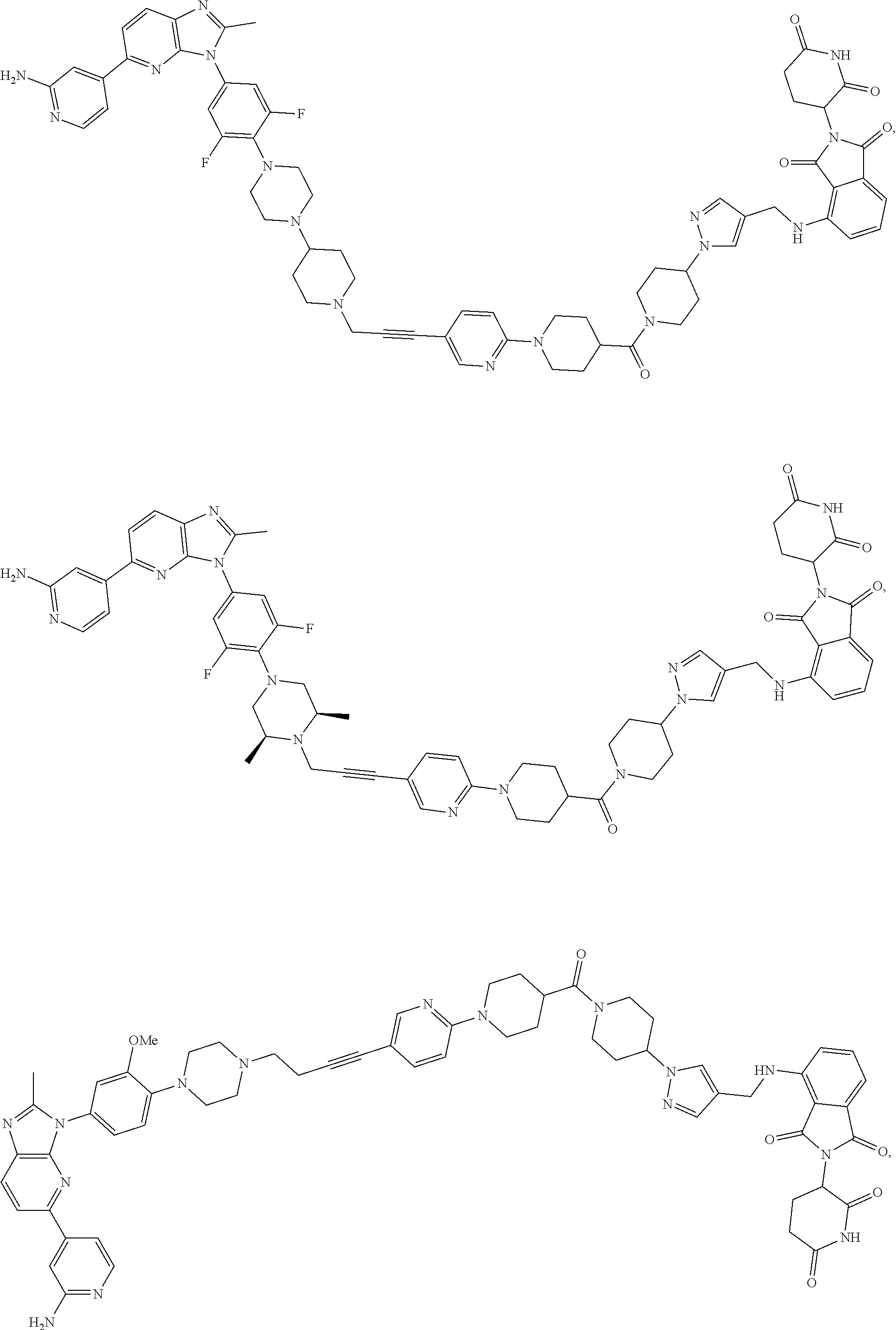 [SMILES: Cc1nc2ccc(-c3ccnc(N)c3)nc2n1-c1cc(F)c(N2CCN(C3CCN(CC#Cc4ccc(N5CCC(C(=O)N6CCC(n7cc(CNc8cccc9c8C(=O)N(C8CCC(=O)NC8=O)C9=O)cn7)CC6)CC5)nc4)CC3)CC2)c(F)c1 ; Cc1nc2ccc(-c3ccnc(N)c3)nc2n1-c1cc(F)c(N2C[C@@H](C)N(CC#Cc3ccc(N4CCC(C(=O)N5CCC(n6cc(CNc7cccc8c7C(=O)N(C7CCC(=O)NC7=O)C8=O)cn6)CC5)CC4)nc3)[C@@H](C)C2)c(F)c1 ; COc1cc(-n2c(C)nc3ccc(-c4ccnc(N)c4)nc32)ccc1N1CCN(CCC#Cc2ccc(N3CCC(C(=O)N4CCC(n5cc(CNc6cccc7c6C(=O)N(C6CCC(=O)NC6=O)C7=O)cn5)CC4)CC3)nc2)CC1]

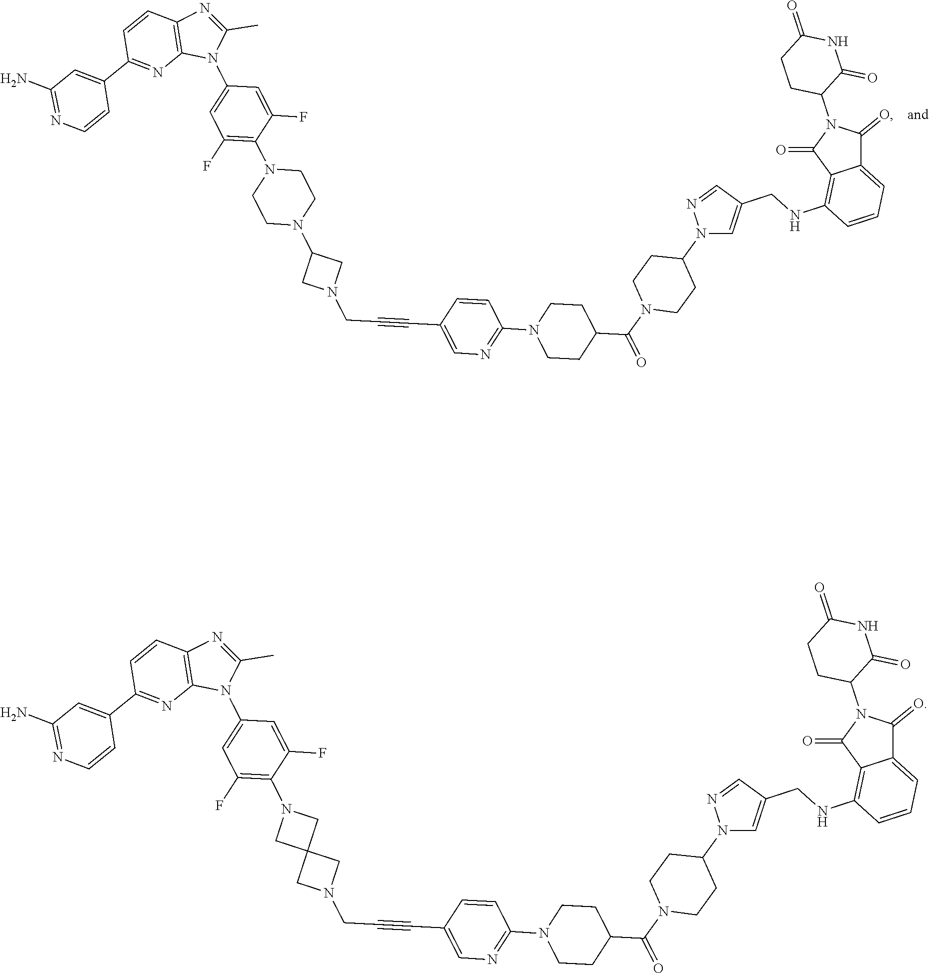 [SMILES: Cc1nc2ccc(-c3ccnc(N)c3)nc2n1-c1cc(F)c(N2CCN(C3CN(CC#Cc4ccc(N5CCC(C(=O)N6CCC(n7cc(CNc8cccc9c8C(=O)N(C8CCC(=O)NC8=O)C9=O)cn7)CC6)CC5)nc4)C3)CC2)c(F)c1 ; Cc1nc2ccc(-c3ccnc(N)c3)nc2n1-c1cc(F)c(N2CC3(CN(CC#Cc4ccc(N5CCC(C(=O)N6CCC(n7cc(CNc8cccc9c8C(=O)N(C8CCC(=O)NC8=O)C9=O)cn7)CC6)CC5)nc4)C3)C2)c(F)c1]

In another aspect, the description provides compositions comprising compounds as described herein, and a pharmaceutically acceptable carrier. In certain embodiments, the compositions are therapeutic or pharmaceutical compositions comprising an effective amount of a compound as described herein and a pharmaceutically acceptable carrier. In certain embodiments, the therapeutic or pharmaceutical compositions comprise an additional biologically active agent, e.g., an agent effective for the treatment of a condition associated with aberrant expression of one or more of DYRK1A, DYRK1B, DYRK2, DYRK3, CLK1, CLK2, CLK3, CLK4, and HASPIN (e.g., Alzheimer's disease, down syndrome, diabetes, an autoimmune disease, an inflammatory disorder (e.g., airway inflammation, osteoarthritis (e.g., knee related osteoarthritis)), cancer (e.g., glioblastoma, prostate cancer, metastatic breast cancer, metastatic lung cancer, multiple myeloma, secondary metastatic tumors of the brain, colorectal cancer), a viral infection (e.g., SARS-COV-2 infection (e.g., COVID-19)), and other diseases).

In any of the aspects or embodiments described herein, the therapeutic compositions comprising compounds described herein can be in any suitable dosage form, e.g., solid, or liquid, and configured to be delivered by any suitable route, e.g., oral, parenteral, intravenous, intraperitoneal, subcutaneous, intramuscular, etc.

In another aspect, the disclosure provides methods of modulating protein ubiquitination and degradation in a subject, e.g., a cell, a tissue, mammal, or human patient, the method comprising administering an effective amount of a compound as described herein or a composition comprising an effective amount of the same to a subject, wherein the compound or composition comprising the same is effective in modulating protein ubiquitination and degradation of the protein in the subject. In certain embodiments, the protein is one or more of DYRK1A, DYRK1B, DYRK2, DYRK3, CLK1, CLK2, CLK3, CLK4, and HASPIN.

In another aspect, the disclosure provides methods of modulating protein ubiquitination and degradation of one or more of DYRK1A, DYRK1B, DYRK2, DYRK3, CLK1, CLK2, CLK3, CLK4, and HASPIN in a subject, e.g., a cell, a tissue, mammal, or human patient, the method comprising administering an effective amount of a compound as described herein or a composition comprising an effective amount of the same to a subject, wherein the compound or composition comprising the same is effective in modulating protein ubiquitination and degradation of one or more of DYRK1A, DYRK1B, DYRK2, DYRK3, CLK1, CLK2, CLK3, CLK4, and HASPIN in the subject.

In another aspect, the disclosure provides methods of treating or ameliorating a symptom of a disease related to one or more of DYRK1A, DYRK1B, DYRK2, DYRK3, CLK1, CLK2, CLK3, CLK4, and HASPIN activity in a subject, e.g., a cell, a tissue, mammal, or human patient, the method comprising administering an effective amount of a compound as described herein or a composition comprising an effective amount of the same to a subject in need thereof, wherein the compound or composition comprising the same is effective in treating or ameliorating a symptom of a disease related to one or more of DYRK1A, DYRK1B, DYRK2, DYRK3, CLK1, CLK2, CLK3, CLK4, and HASPIN activity in the subject. In a preferred embodiment, the subject is a human.

In another aspect, the disclosure provides methods for identifying the effects of the degradation of proteins of interest in a biological system (e.g., one or more of DYRK1A, DYRK1B, DYRK2, DYRK3, CLK1, CLK2, CLK3, CLK4, and HASPIN) using compounds according to the present invention.

In certain embodiments, the present invention provides methods for administering an effective amount of a compound as described herein or a composition comprising an effective amount of the same (alone or with additional agents (e.g., remdesivir)) to a subject (e.g., a human subject) (e.g., a human subject suffering from or at risk of suffering from viral infection) (a human subject suffering from or at risk of suffering from a condition related to a viral infection for purposes of treating, preventing and/or ameliorating the viral infection and/or symptoms of a viral infection (e.g., SARS-COV-2 infection (e.g., COVID-19)). In such embodiments, administration results in suppression of pro-inflammatory cytokine activity (e.g., IL-6 activity) within the subject. In some embodiments, administration results in enhancement of NK cell activity within the subject. In some embodiments, administration results in enhancement of neutrophil activity within the subject. In some embodiments, administration results in inhibition of viral entry into the subject's cells through inhibiting binding of the virus with heparin sulfate proteoglycan within such cells.

In such embodiments, the methods are not limited to treating, preventing and/or ameliorating a particular type or kind of viral infection or symptoms of a particular type or kind of viral infection. In some embodiments, the viral infection is a SARS-COV-2 related viral infection (e.g., COVID-19). In some embodiments, the viral infection is any infection related to influenza, HIV, HIV-1, HIV-2, drug-resistant HIV, Junin virus, Chikungunya virus, Yellow Fever virus, Dengue virus, Pichinde virus, Lassa virus, adenovirus, Measles virus, Punta Toro virus,

Respiratory Syncytial virus, Rift Valley virus, RHDV, SARS coronavirus, Tacaribe virus, and West Nile virus. In some embodiments, the viral infection is associated with any virals having M^{pro} protease activity and/or expression.

In some embodiments, the one or more symptoms related to viral infection includes, but is not limited to, fever, fatigue, dry cough, myalgias, dyspnea, acute respiratory distress syndrome, and pneumonia.

In another aspect, the description provides kits comprising compounds or compositions as described herein. The kit may be promoted, distributed, or sold as a unit for performing the methods of the present invention. In addition, the kits of the present invention may preferably contain instructions which describe a suitable use. Such kits can be conveniently used, e.g., in clinical settings, to treat patients.

Where applicable or not specifically disclaimed, any one of the embodiments described herein are contemplated to be able to combine with any other one or more embodiments, even though the embodiments are described under different aspects of the invention. As such, the preceding general areas of utility are given by way of example only and are not intended to be limiting on the scope of the present disclosure and appended claims. Additional objects and advantages associated with the compositions, methods, and processes of the present invention will be appreciated by one of ordinary skill in the art in light of the instant claims, description, and examples. For example, the various aspects and embodiments of the invention may be utilized in numerous combinations, all of which are expressly contemplated by the present description. These additional advantages objects and embodiments are expressly included within the scope of the present invention. The publications and other materials used herein to illuminate the background of the invention, and in particular cases, to provide additional details respecting the practice, are incorporated by reference.

### BRIEF DESCRIPTION OF THE DRAWINGS

FIG. 1 shows chiral HPLC properties of (R)-4-(((1-(1-(1-(5-(3-(4-(4-(5-(2-aminopyridin-4-yl)-2-methyl-3H-imidazo[4,5-b]pyridin-3-yl)-2-fluorophenyl)piperazin-1-yl)prop-1-yn-1-yl)pyridin-2-yl)piperidine-4-carbonyl)piperidin-4-yl)-1H-pyrazol-4-yl)methyl)amino)-2-(2,6-dioxopiperidin-3-yl)isoindoline-1,3-dione (29).

FIG. 2 shows chiral HPLC properties of(S)-4-(((1-(1-(1-(5-(3-(4-(4-(5-(2-aminopyridin-4-yl)-2-methyl-3H-imidazo[4,5-b]pyridin-3-yl)-2-fluorophenyl)piperazin-1-yl)prop-1-yn-1-yl)pyridin-2-yl)piperidine-4-carbonyl)piperidin-4-yl)-1H-pyrazol-4-yl)methyl)amino)-2-(2,6-dioxopiperidin-3-yl)isoindoline-1,3-dione (30).

### Definitions

The following terms are used to describe the present invention. In instances where a term is not specifically defined herein, that term is given an art-recognized meaning by those of ordinary skill applying that term in context to its use in describing the present invention.

The articles “a” and “an” as used herein and in the appended claims are used herein to refer to one or to more than one (i.e., to at least one) of the grammatical object of the article unless the context clearly indicates otherwise. By way of example, “an element” means one element or more than one element.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of′ or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

The term “about” and the like, as used herein, in association with numeric values or ranges, reflects the fact that there is a certain level of variation that is recognized and tolerated in the art due to practical and/or theoretical limitations. For example, minor variation is tolerated due to inherent variances in the manner in which certain devices operate and/or measurements are taken. In accordance with the above, the phrase “about” is normally used to encompass values within the standard deviation or standard error.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

As used herein in the specification and in the claims, the phrase “at least one,” in reference to a list of one or more elements, should be understood to mean at least one element selected from anyone or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a nonlimiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, in certain methods described herein that include more than one step or act, the order of the steps or acts of the method is not necessarily limited to the order in which the steps or acts of the method are recited unless the context indicates otherwise.

The terms “co-administration” and “co-administering” or “combination therapy” can refer to both concurrent administration (administration of two or more therapeutic agents at the same time) and time varied administration (administration of one or more therapeutic agents at a time different from that of the administration of an additional therapeutic agent or agents), as long as the therapeutic agents are present in the patient to some extent, preferably at effective amounts, at the same time. In certain preferred aspects, one or more of the present compounds described herein, are co-administered in combination with at least one additional bioactive agent, especially including an anticancer agent. In particularly preferred aspects, the co-administration of compounds results in synergistic activity and/or therapy, including anticancer activity.

The term “effective” can mean, but is in no way limited to, that amount/dose of the active pharmaceutical ingredient, which, when used in the context of its intended use, effectuates or is sufficient to prevent, inhibit the occurrence, ameliorate, delay or treat (alleviate a symptom to some extent, preferably all) the symptoms of a condition, disorder or disease state in a subject in need of such treatment or receiving such treatment. The term effective subsumes all other effective amount or effective concentration terms, e.g., “effective amount/dose,” “pharmaceutically effective amount/dose” or “therapeutically effective amount/dose,” which are otherwise described or used in the present application.

The effective amount depends on the type and severity of disease, the composition used, the route of administration, the type of mammal being treated, the physical characteristics of the specific mammal under consideration, concurrent medication, and other factors which those skilled in the medical arts will recognize. The exact amount can be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

The term “pharmacological composition,” “therapeutic composition,” “therapeutic formulation” or “pharmaceutically acceptable formulation” can mean, but is in no way limited to, a composition or formulation that allows for the effective distribution of an agent provided by the invention, which is in a form suitable for administration to the physical location most suitable for their desired activity, e.g., systemic administration.

The term “pharmaceutically acceptable” or “pharmacologically acceptable” can mean, but is in no way limited to, entities and compositions that do not produce an adverse, allergic or other untoward reaction when administered to an animal, or a human, as appropriate.

The term “pharmaceutically acceptable carrier” or “pharmacologically acceptable carrier” can mean, but is in no way limited to, any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, Ringer's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The term “systemic administration” refers to a route of administration that is, e.g., enteral or parenteral, and results in the systemic distribution of an agent leading to systemic absorption or accumulation of drugs in the blood stream followed by distribution throughout the entire body. Suitable forms, in part, depend upon the use or the route of entry, for example oral, transdermal, or by injection. Such forms should not prevent the composition or formulation from reaching a target cell (i.e., a cell to which the negatively charged polymer is desired to be delivered to). For example, pharmacological compositions injected into the blood stream should be soluble. Other factors are known in the art, and include considerations such as toxicity and forms which prevent the composition or formulation from exerting its effect. Administration routes which lead to systemic absorption include, without limitations: intravenous, subcutaneous, intraperitoneal, inhalation, oral, intrapulmonary and intramuscular. The rate of entry of a drug into the circulation has been shown to be a function of molecular weight or size. The use of a liposome or other drug carrier comprising the compounds of the instant invention can potentially localize the drug, for example, in certain tissue types, such as the tissues of the reticular endothelial system (RES). A liposome formulation which can facilitate the association of drug with the surface of cells, such as, lymphocytes and macrophages is also useful.

The term “local administration” refers to a route of administration in which the agent is delivered to a site that is apposite or proximal, e.g., within about 10 cm, to the site of the lesion or disease.

The term “compound”, as used herein, unless otherwise indicated, refers to any specific chemical compound disclosed herein and includes tautomers, regioisomers, geometric isomers, and where applicable, stereoisomers, including optical isomers (enantiomers) and other stereoisomers (diastereomers) thereof, as well as pharmaceutically acceptable salts and derivatives (including prodrug forms) thereof where applicable, in context. Within its use in context, the term compound generally refers to a single compound, but also may include other compounds such as stereoisomers, regioisomers and/or optical isomers (including racemic mixtures) as well as specific enantiomers or enantiomerically enriched mixtures of disclosed compounds. The term also refers, in context to prodrug forms of compounds which have been modified to facilitate the administration and delivery of compounds to a site of activity. It is noted that in describing the present compounds, numerous substituents and variables associated with same, among others, are described.

It is understood by those of ordinary skill that molecules which are described herein are stable compounds as generally described hereunder. When the bond “=” is shown, both a double bond and single bond are represented within the context of the compound shown.

As used herein, “derivatives” can mean compositions formed from the native compounds either directly, by modification, or by partial substitution. As used herein, “analogs” can mean compositions that have a structure similar to, but not identical to, the native compound.

The term “Ubiquitin Ligase” refers to a family of proteins that facilitate the transfer of ubiquitin to a specific substrate protein, targeting the substrate protein for degradation. For example, Von Hippel-Lindau E3 Ubiquitin Ligase or VCB E3 Ubiquitin Ligase is protein that alone or in combination with an E2 ubiquitin-conjugating enzyme causes the attachment of ubiquitin to a lysine on a target protein, and subsequently targets the specific protein substrates for degradation by the proteasome. Thus, E3 ubiquitin ligase alone or in complex with an E2 ubiquitin conjugating enzyme is responsible for the transfer of ubiquitin to targeted proteins. In general, the ubiquitin ligase is involved in polyubiquitination such that a second ubiquitin is attached to the first; a third is attached to the second, and so forth. Polyubiquitination marks proteins for degradation by the proteasome. However, there are some ubiquitination events that are limited to mono-ubiquitination, in which only a single ubiquitin is added by the ubiquitin ligase to a substrate molecule. Mono-ubiquitinated proteins are not targeted to the proteasome for degradation, but may instead be altered in their cellular location or function, for example, via binding other proteins that have domains capable of binding ubiquitin. Further complicating matters, different lysines on ubiquitin can be targeted by an E3 to make chains. The most common lysine is Lys48 on the ubiquitin chain. This is the lysine used to make polyubiquitin, which is recognized by the proteasome.

The term “subject” is used throughout the specification to describe a cell, tissue, or animal, preferably a human or a domesticated animal, to whom treatment, including prophylactic treatment, with the compositions according to the present invention is provided. For treatment of those infections, conditions or disease states which are specific for a specific animal such as a human patient, the term patient refers to that specific animal, including a domesticated animal such as a dog or cat or a farm animal such as a horse, cow, sheep, etc. In general, in the present invention, the term patient refers to a human patient unless otherwise stated or implied from the context of the use of the term.

### DETAILED DESCRIPTION OF THE INVENTION

The following is a detailed description provided to aid those skilled in the art in practicing the present invention. Those of ordinary skill in the art may make modifications and variations in the embodiments described herein without departing from the spirit or scope of the present disclosure. All publications, patent applications, patents, figures and other references mentioned herein are expressly incorporated by reference in their entirety.

Unless otherwise defined, all technical and scientific terms used herein have the same meaning as commonly understood by one of ordinary skill in the art to which this invention belongs. The terminology used in the description is for describing particular embodiments only and is not intended to be limiting of the invention.

Where a range of values is provided, it is understood that each intervening value, to the tenth of the unit of the lower limit unless the context clearly dictates otherwise (such as in the case of a group containing a number of carbon atoms in which case each carbon atom number falling within the range is provided), between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

The DYRK family contains 5 kinases (DYRK1A, DYRK1B, DYRK2, DYRK3 and DYRK4). DYRKs belong to the CMGC group of proline-directed kinases, which also includes cyclin-dependent kinases (CDKs), mitogen-activated protein kinases (MAPKs), glycogen synthase kinases (GSKs) and CDC2-like kinases (CLKs). While the signaling pathways of CDK and MAPK families have been extensively studied, much less is known on how DYRKs and CLKs are linked to other proteins and various physiological or pathological processes. The CLK family comprises CLK1 through CLK4.

The DYRK1A gene is located on chromosome 21 (21q22.2), a region known as the Down-Syndrome Critical Region (DSCR) (see, e.g., Hämmerle et al., 2011 Development 138, 2543-2554). The under- or over-expression of the Dyrk1a gene in mammals or of its orthologous gene minibrain (mnb) in Drosophila causes severe retardation of central nervous system development and maturation. At the molecular level, DYRK1A phosphorylates the nuclear factor of activated T cells (NFAT), counteracting the effect of calcium signaling and maintaining inactive NFAT (see, e.g., Arron et al., 2006 Nature 411, 595-600). DYRK1A has been identified as a negative regulator of the cell cycle that promotes the switch to a quiescent state or differentiation (see, e.g., Chen et al., 2013 Mol. Cell 52, 87-100). In malignant cells, DYRK1A promotes survival via inhibition of pro-apoptotic proteins (see, e.g., Guo et al., 2010 J. Bio. Chem. 285, 13223-13232; Seifert et al., 2008 FEBS J. 275, 6268-6280).

Experiments conducted during the course of developing embodiments for the present invention designed, synthesized and biologically evaluated various Protac compounds capable of inhibiting one or more of DYRK1A, DYRK1B, DYRK2, DYRK3, and CLK1, CLK2, CLK3, CLK4, and HASPIN.

Accordingly, the present invention relates to bifunctional compounds, which find utility to degrade and (inhibit) one or more of the following kinases: DYRK1A, DYRK1B, DYRK2, DYRK3, CLK1, CLK2, CLK3, CLK4, and HASPIN. In particular, the present invention is directed to compounds, which contain on one end an E3 ubiquitin ligase binding moiety which binds to an E3 ubiquitin ligase and on the other end a moiety which binds one or more of the following kinases: DYRK1A, DYRK1B, DYRK2, DYRK3, CLK1, CLK2, CLK3, CLK4, and HASPIN, such that the one or more kinases is placed in proximity to the ubiquitin ligase to effect degradation (and inhibition) of the one or more kinases. The bifunctional compounds serve as therapeutics for the treatment of Alzheimer's disease, down syndrome, diabetes, an autoimmune disease, an inflammatory disorder (e.g., airway inflammation, osteoarthritis (e.g., knee related osteoarthritis)), cancer (e.g., glioblastoma, prostate cancer, metastatic breast cancer, metastatic lung cancer, multiple myeloma, secondary metastatic tumors of the brain, colorectal cancer), a viral infection (e.g., SARS-COV-2 infection (e.g., COVID-19)), and other diseases.

In one aspect, the present invention provides compounds useful for regulating activity of one or more of the following kinases: DYRK1A, DYRK1B, DYRK2, DYRK3, CLK1, CLK2, CLK3, CLK4, and HASPIN. The composition comprises a ubiquitin pathway protein binding moiety (preferably for an E3 ubiquitin ligase, alone or in complex with an E2 ubiquitin conjugating enzyme which is responsible for the transfer of ubiquitin to targeted proteins) according to a defined chemical structure and a protein targeting moiety which are linked or coupled together, preferably through a linker, wherein the ubiquitin pathway protein binding moiety recognizes an ubiquitin pathway protein and the targeting moiety recognizes one or more of the noted kinases.

In certain embodiments, the compounds have the following general structure: Target Binding Moiety (TBM)-L-ULM or ULM-L-TBM or TBM-ULM.

In some embodiments, the TBM is a small molecule inhibitor of one or more of DYRK1A, DYRK1B, DYRK2, DYRK3, CLK1, CLK2, CLK3, CLK4, and HASPIN, ULM is an E3 ligase binding moiety (e.g., a VHL binding moiety, a cereblon binding moiety, a XIAP binding moiety, or a MDM2 binding moiety), and L is a bond or a linker moiety which links the TBM and ULM.

In certain embodiments, the small molecule inhibitor of one or more of DYRK1A, DYRK1B, DYRK2, DYRK3, CLK1, CLK2, CLK3, CLK4, and HASPIN is selected from 4-(3-(3-fluoro-4-(4-methylpiperazin-1-yl)phenyl)-2-methyl-3H-imidazo[4,5-b]pyridin-5-yl)pyridin-2-amine

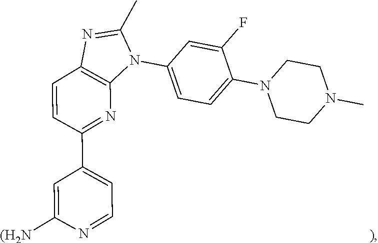

4-(3-(3-methoxy-4-(4-methylpiperazin-1-yl)phenyl)-2-methyl-3H-imidazo[4,5-b]pyridin-5-yl)pyridin-2-amine

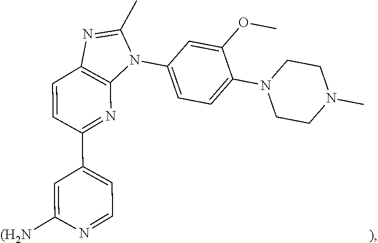

4-(3-(3,5-difluoro-4-(4-(1-methylazetidin-3-yl)piperazin-1-yl)phenyl)-2-methyl-3H-imidazo[4,5-b]pyridin-5-yl)pyridin-2-amine

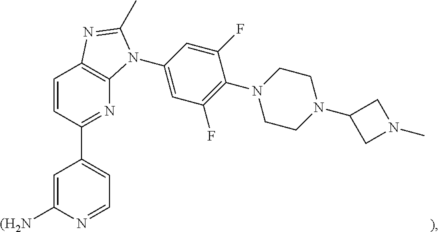

4-(3-(3,5-difluoro-4-(6-methyl-2,6-diazaspiro[3.3]heptan-2-yl)phenyl)-2-methyl-3H-imidazo[4,5-b]pyridin-5-yl)pyridin-2-amine

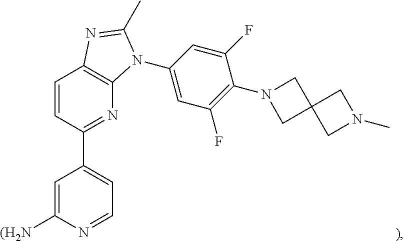

4-(3-(3,5-difluoro-4-((3S,5R)-3,4,5-trimethylpiperazin-1-yl)phenyl)-2-methyl-3H-imidazo[4,5-b]pyridin-5-yl)pyridin-2-amine

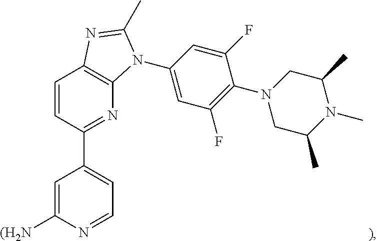

4-(3-(3,5-difluoro-4-(4-(1-methylpiperidin-4-yl)piperazin-1-yl)phenyl)-2-methyl-3H-imidazo[4,5-b]pyridin-5-yl)pyridin-2-amine

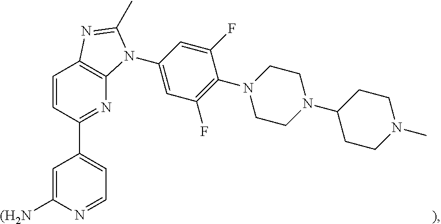

4-(3-(3-fluoro-4-((1S,4S)-5-methyl-2,5-diazabicyclo[2.2.1]heptan-2-yl)phenyl)-2-methyl-3H-imidazo[4,5-b]pyridin-5-yl)pyridin-2-amine

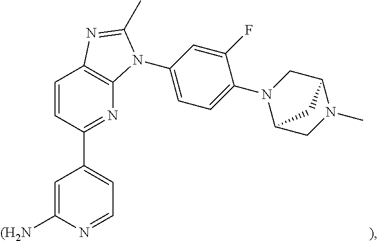

4-(3-(3-fluoro-4-(3-methyl-3,8-diazabicyclo[3.2.1]octan-8-yl)phenyl)-2-methyl-3H-imidazo[4,5-b]pyridin-5-yl)pyridin-2-amine

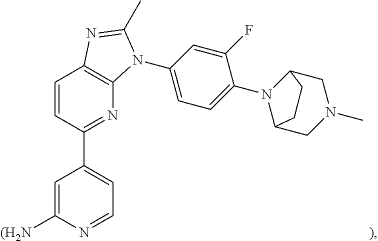

4-(3-(3-fluoro-4-((3aR,6aS)-5-methylhexahydropyrrolo[3,4-c]pyrrol-2 (1H)-yl)phenyl)-2-methyl-3H-imidazo[4,5-b]pyridin-5-yl)pyridin-2-amine

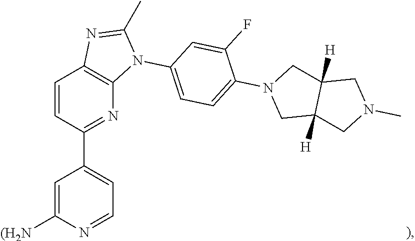

4-(3-(3,5-difluoro-4-(4-methylpiperazin-1-yl)phenyl)-2-methyl-3H-imidazo[4,5-b]pyridin-5-yl)pyridin-2-amine

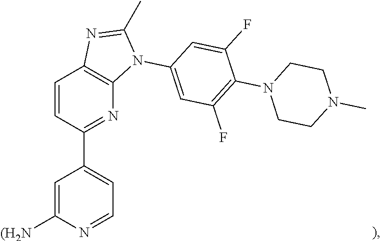

4-(2-methyl-3-(4-(4-methylpiperazin-1-yl)-3-(trifluoromethyl)phenyl)-3H-imidazo[4,5-b]pyridin-5-yl)pyridin-2-amine

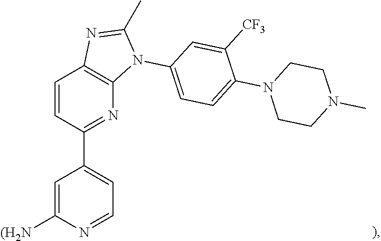

4-(2-methyl-3-(4-((4-methylpiperazin-1-yl)methyl)phenyl)-3H-imidazo[4,5-b]pyridin-5-yl)pyridin-2-amine

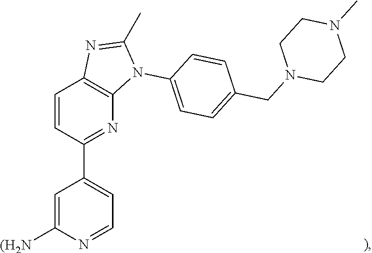

and 4-(3-(3,5-dichloro-4-(4-methylpiperazin-1-yl)phenyl)-2-methyl-3H-imidazo[4,5-b]pyridin-5-yl)pyridin-2-amine

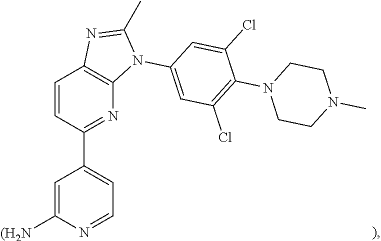

In some embodiments, the TBM covalently couples with the linker at any location within the TBM. In some embodiments related to the small molecule inhibitors of one or more of DYRK1A, DYRK1B, DYRK2, DYRK3, CLK1, CLK2, CLK3, CLK4, and HASPIN, the TBM covalently couples with the linker at the following “ [symbol] ”location:

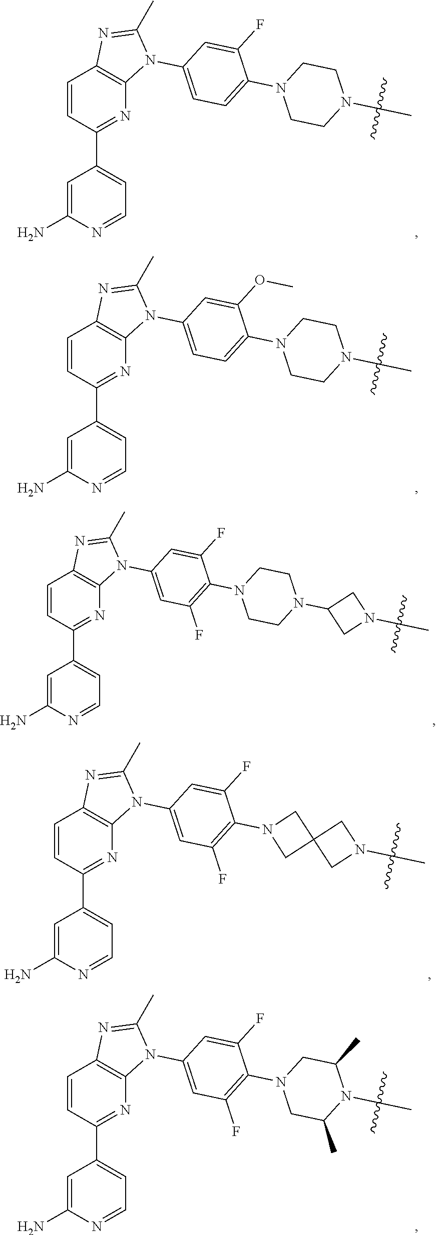

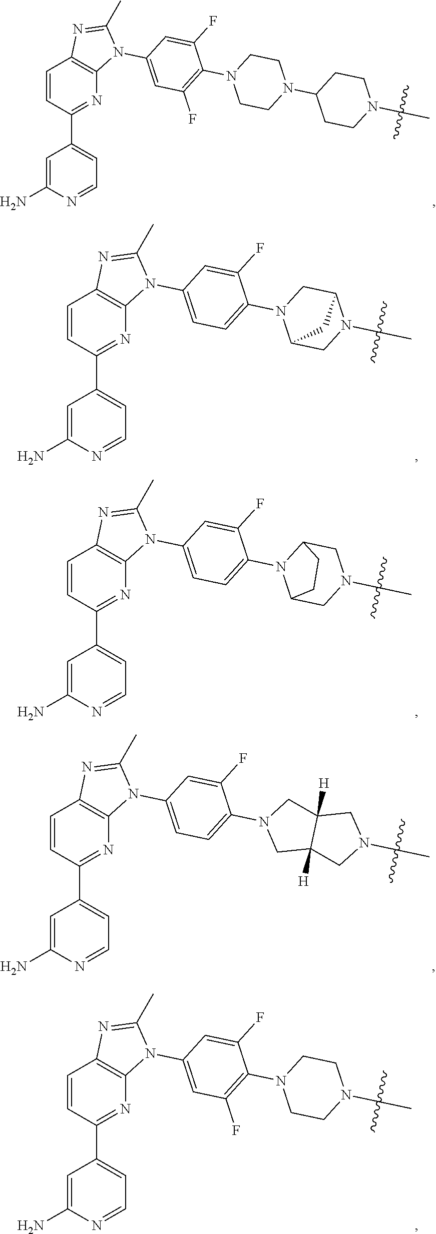

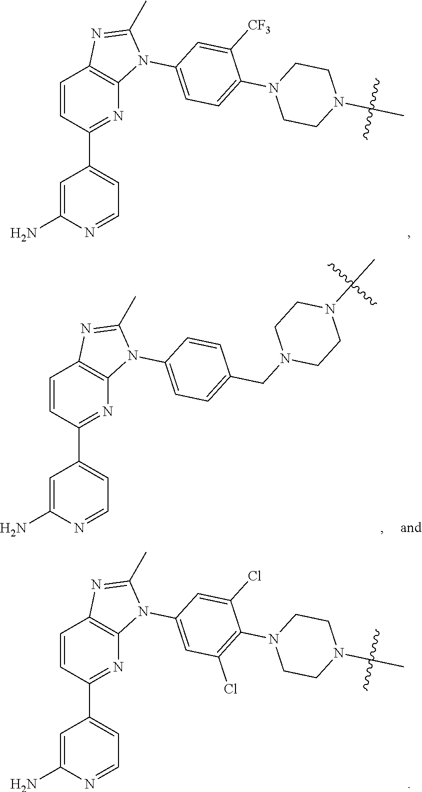 [SMILES: *N1CCN(c2ccc(-n3c(C)nc4ccc(-c5ccnc(N)c5)nc43)cc2C(F)(F)F)CC1 ; *N1CCN(Cc2ccc(-n3c(C)nc4ccc(-c5ccnc(N)c5)nc43)cc2)CC1 ; *N1CCN(c2c(Cl)cc(-n3c(C)nc4ccc(-c5ccnc(N)c5)nc43)cc2Cl)CC1]

In some embodiments, the ULM covalently couples with the linker at any location within the ULM. For example, in some embodiments, the ULM covalently couples with the linker at the following “ [symbol] ” location: VHL

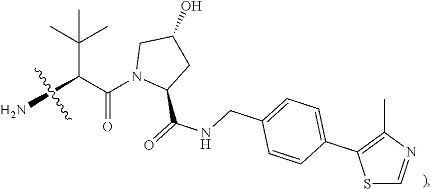

cereblon

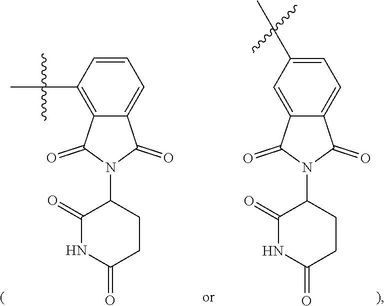 [SMILES: *c1cccc2c1C(=O)N(C1CCC(=O)NC1=O)C2=O ; *c1ccc2c(c1)C(=O)N(C1CCC(=O)NC1=O)C2=O]

2-(2,6-dioxopiperidin-3-yl)-5-fluoroisoindoline-1,3-dione

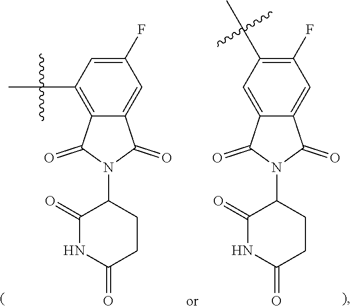

3-(1-oxoisoindolin-2-yl)piperidine-2,6-dione

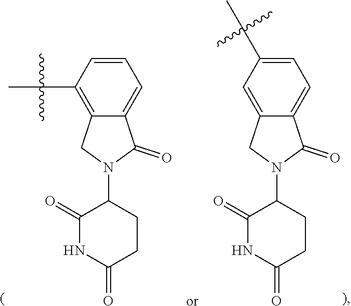

2-(1-methyl-2,6-dioxopiperidin-3-yl)isoindoline-1,3-dione

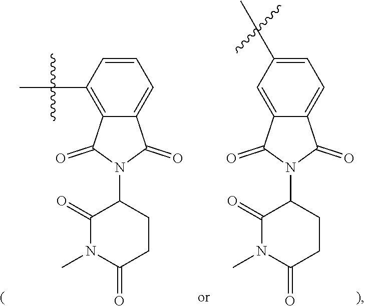 [SMILES: *c1cccc2c1C(=O)N(C1CCC(=O)N(C)C1=O)C2=O ; *c1ccc2c(c1)C(=O)N(C1CCC(=O)N(C)C1=O)C2=O]

and 3-phenylpiperidine-2,6-dione

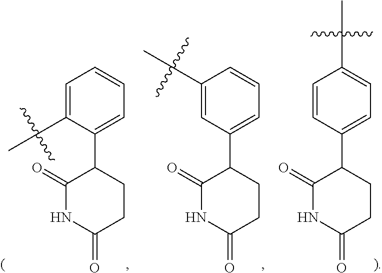

As such, in certain embodiments, the description provides compounds having one of the following general structures: TBM-L-VHL binding moiety, TBM-L-Cereblon binding moiety, TBM-L-XIAP binding moiety, and TBM-L-MDM2 binding moiety.

In certain embodiments, the linker is a bond or a chemical linker that covalently couples the TBM and the ULM.

In some embodiments, the linker is represented by:

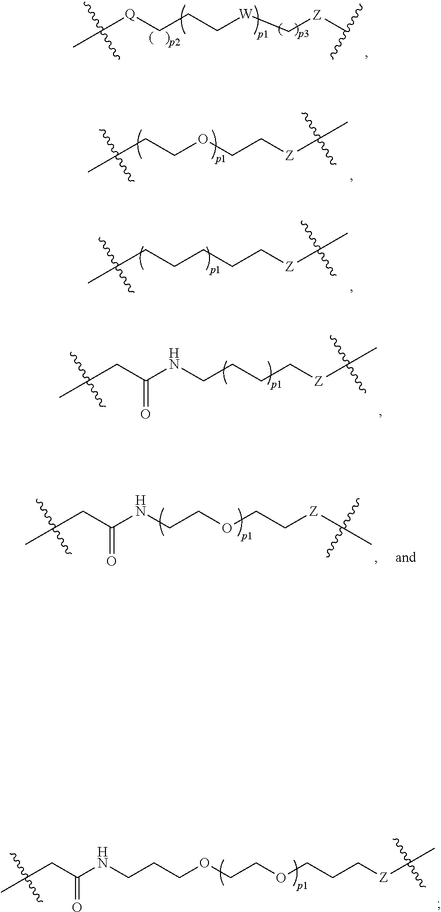

or an enantiomer, diastereomer, or stereoisomer thereof, wherein p1 is an integer selected from 0 to 12; p2 is an integer selected from 0 to 12; p3 is an integer selected from 1 to 6; each W is independently absent, CH₂, O, S, NH, or NR⁸; Z is absent, CH₂, O, NH, or NR⁸; each R⁸ is independently C₁-C₃ alkyl; and Q is absent or CH₂C(O)NH.

In some embodiments, the linker is selected from:

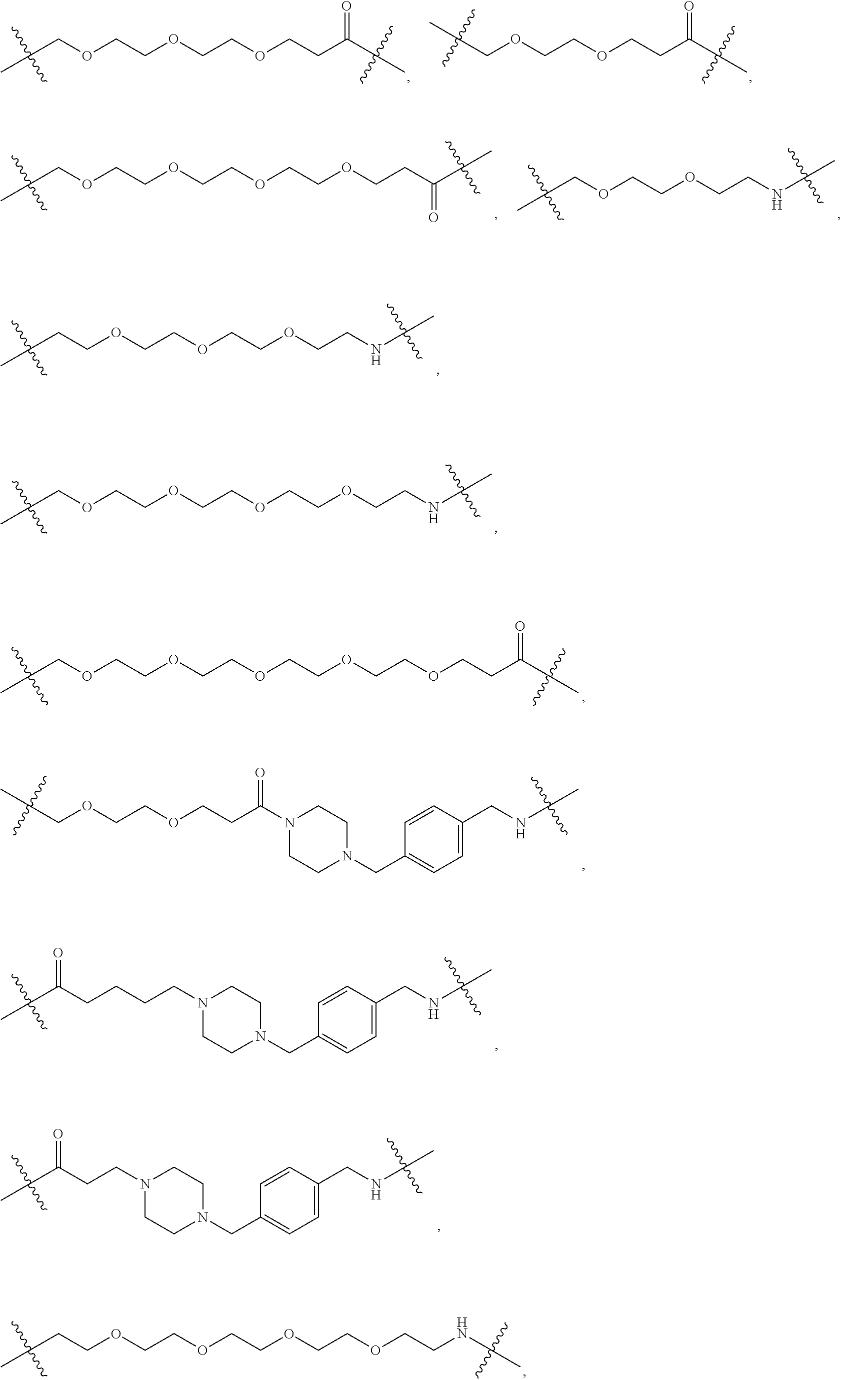

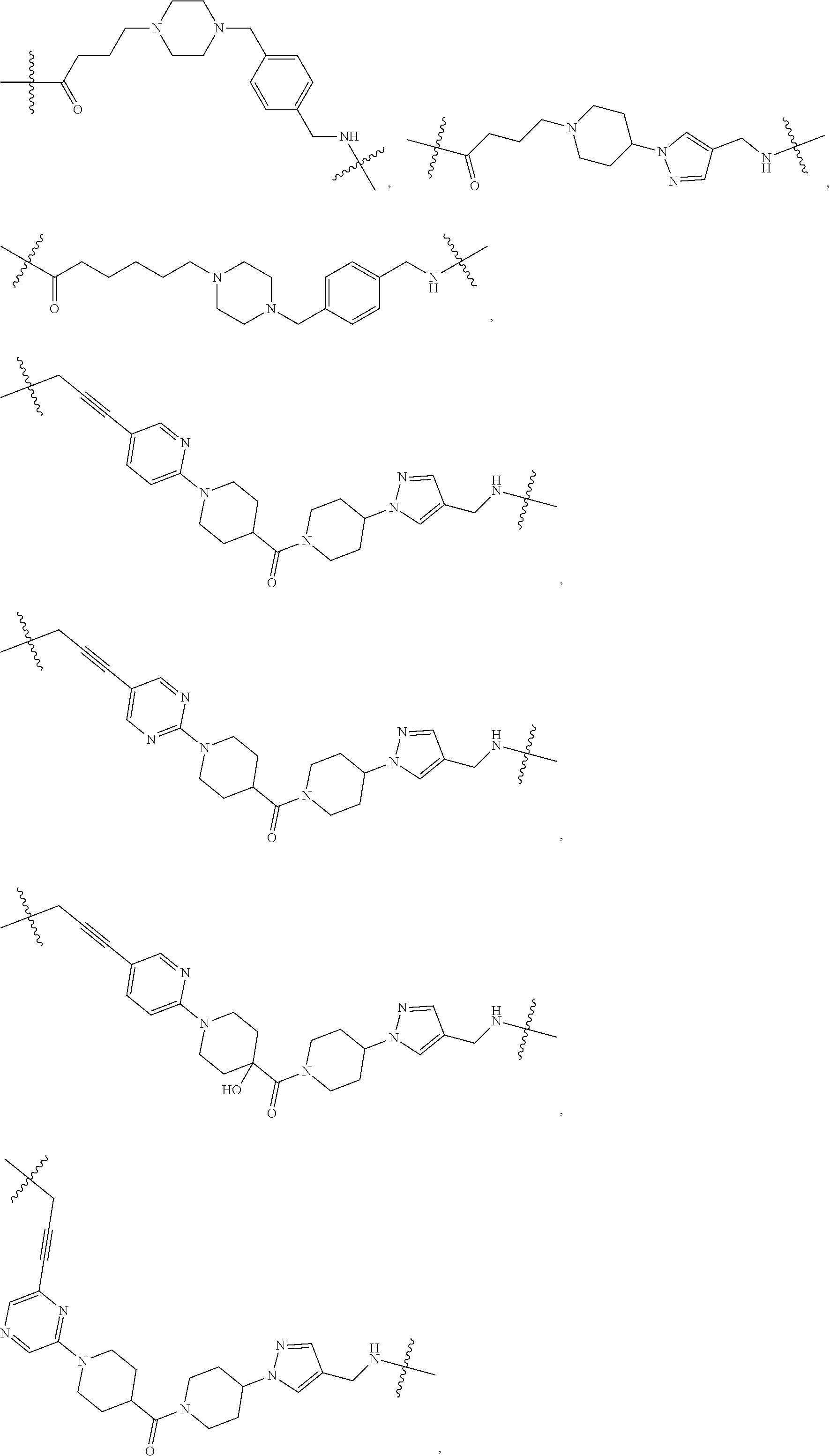

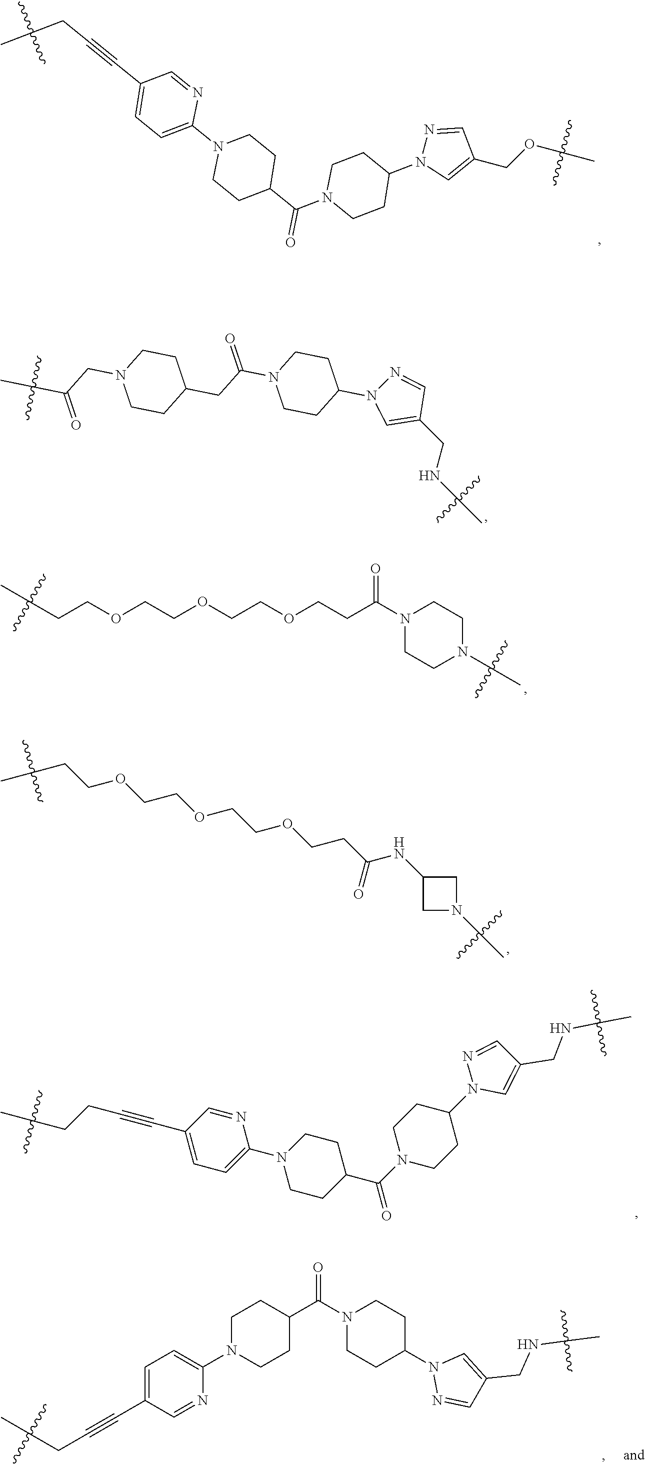 [SMILES: *CC#Cc1ccc(N2CCC(C(=O)N3CCC(n4cc(CO*)cn4)CC3)CC2)nc1 ; *NCc1cnn(C2CCN(C(=O)CC3CCN(CC(*)=O)CC3)CC2)c1 ; *CCOCCOCCOCCC(=O)N1CCN(*)CC1 ; *CCOCCOCCOCCC(=O)NC1CN(*)C1 ; *CCC#Cc1ccc(N2CCC(C(=O)N3CCC(n4cc(CN*)cn4)CC3)CC2)nc1 ; *CC#Cc1ccc(N2CCC(C(=O)N3CCC(n4cc(CN*)cn4)CC3)CC2)nc1]

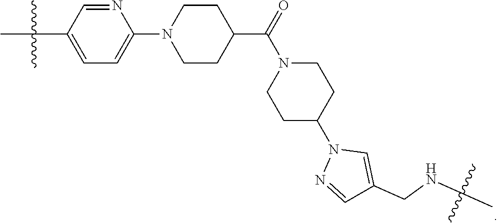 [SMILES: *NCc1cnn(C2CCN(C(=O)C3CCN(c4ccc(*)cn4)CC3)CC2)c1]

It will be understood that the general structures are exemplary and the respective moieties can be arranged spatially in any desired order or configuration, e.g., ULM-L-TBM, TBM-L-ULM, TMB-ULM, ULM-TBM, respectively.

In some embodiments, TBM-L-ULM is represented by TBM-A-B-modified cereblon, wherein A is represented by

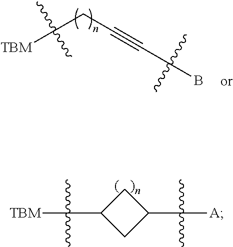

wherein B is represented by

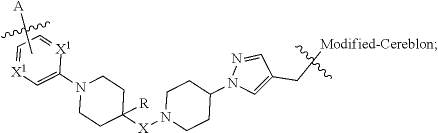

wherein modified-cereblon is selected from:

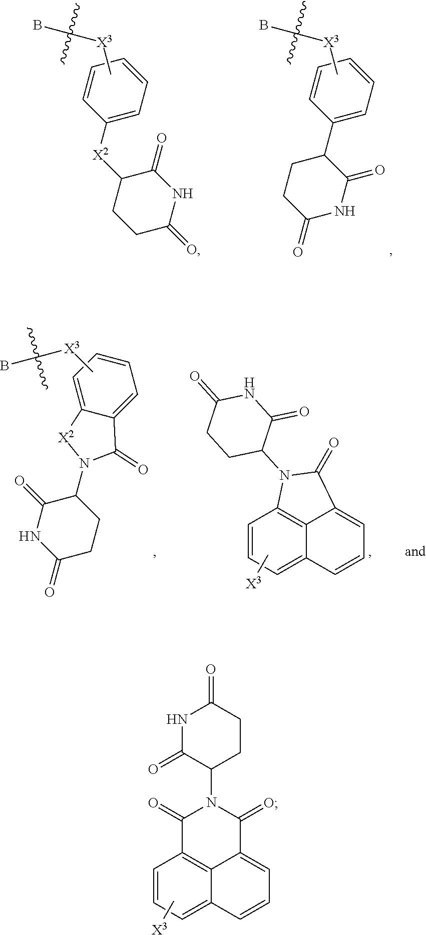

wherein n is 0, 1 or 2; wherein R is OH or H; wherein X is CH₂ or CO; wherein each X¹ is either N or CH; wherein X² is CO, CH₂, or NH; wherein X³ is NH or O.

It will be understood that the general structures are exemplary and the respective moieties can be arranged spatially in any desired order or configuration, e.g., ULM-L-TBM, TBM-L-ULM, TBM-ULM, ULM-TBM respectively. 5

In certain additional embodiments, the compounds comprise a plurality of E3 ligase binding moieties and/or a plurality of TBMs.

In certain embodiments, the description provides a bifunctional compound having a structure as described herein, a salt, a polymorph, and a prodrug thereof.

In certain embodiments, the bifunctional compound is selected from:

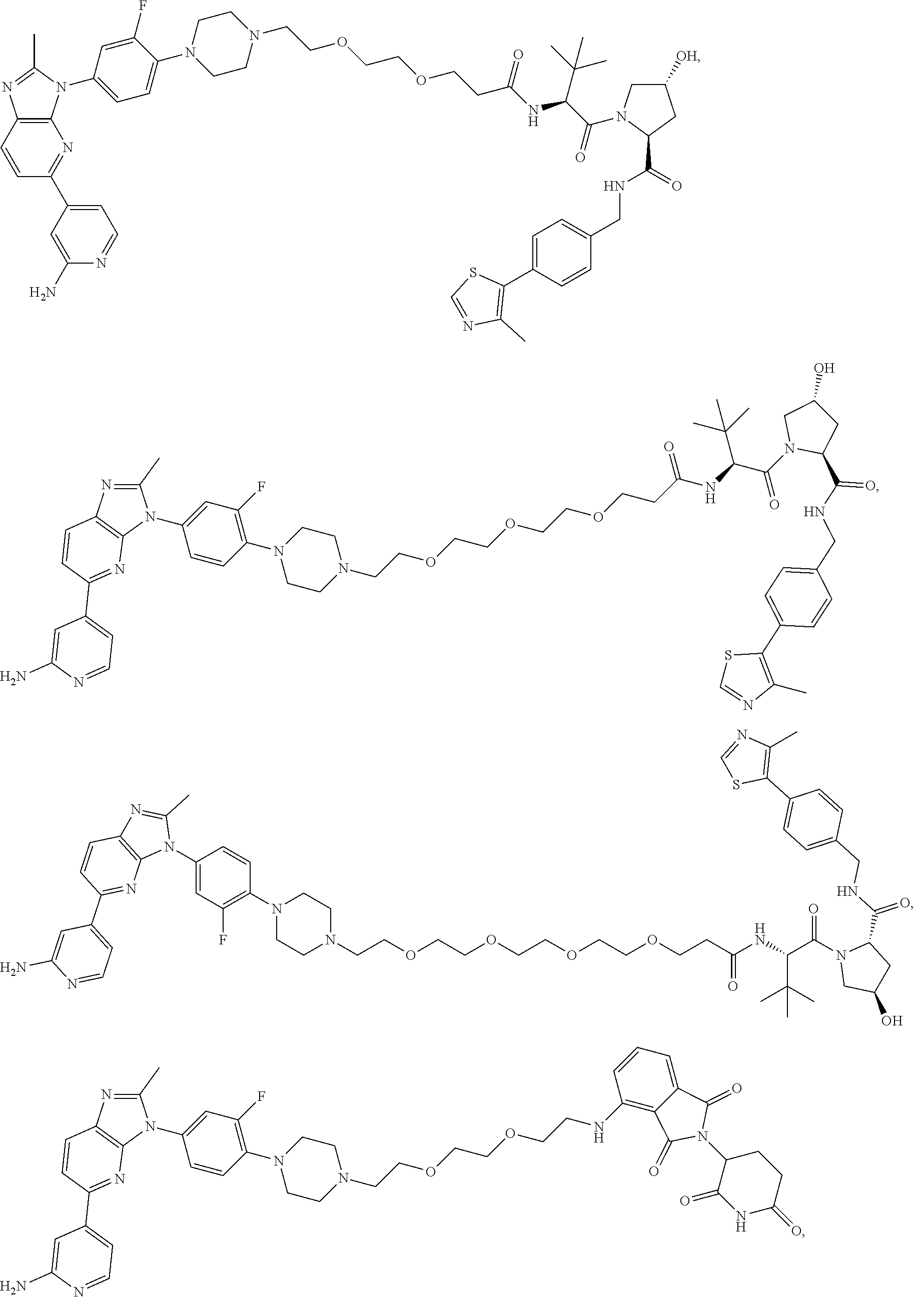

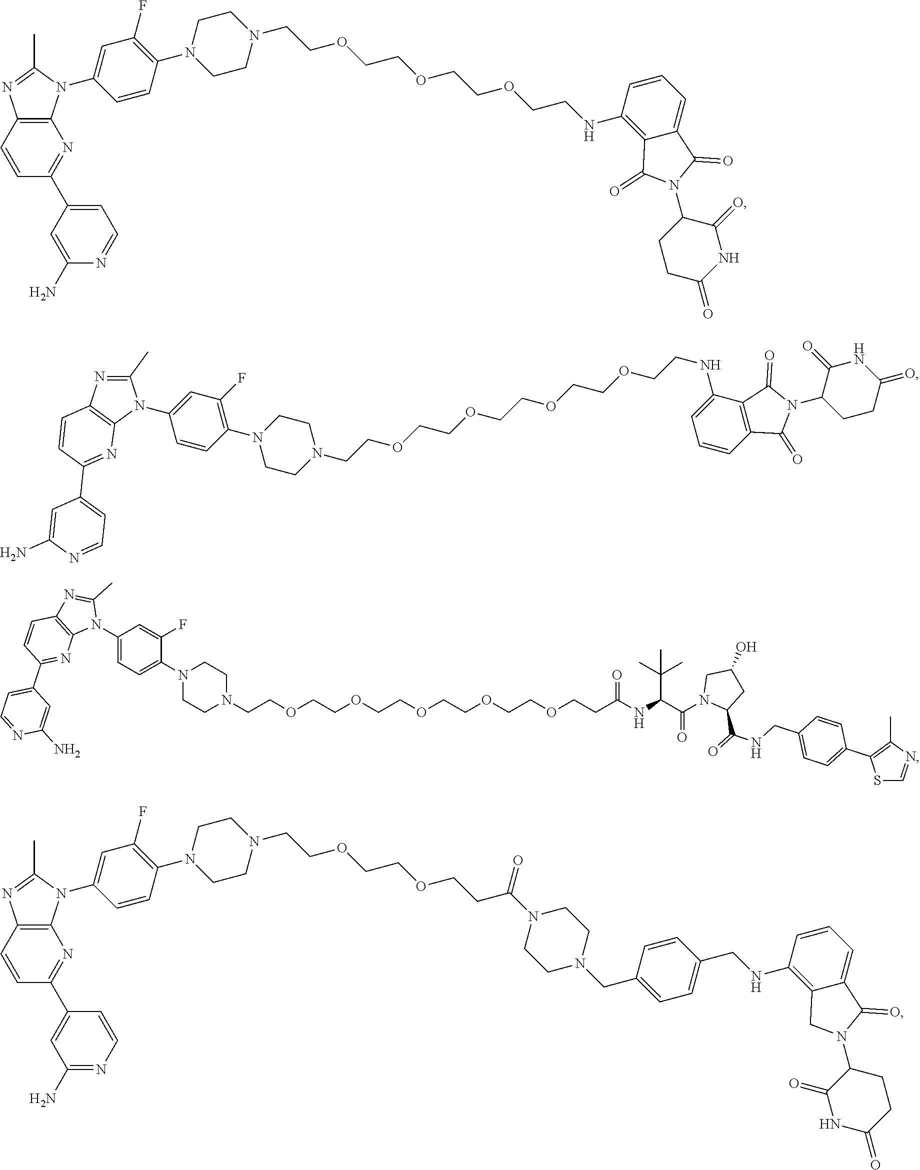

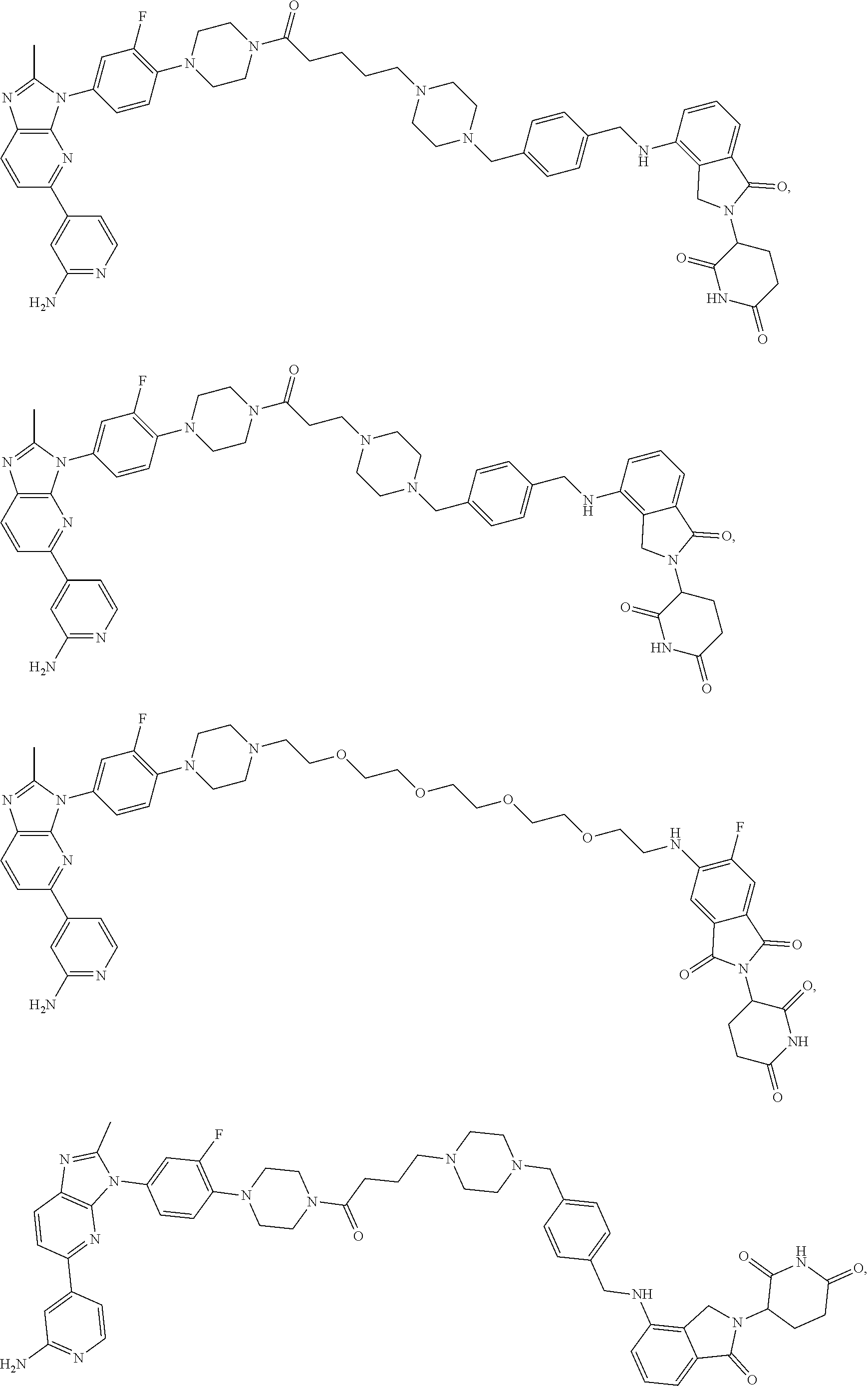 [SMILES: Cc1nc2ccc(-c3ccnc(N)c3)nc2n1-c1ccc(N2CCN(C(=O)CCCCN3CCN(Cc4ccc(CNc5cccc6c5CN(C5CCC(=O)NC5=O)C6=O)cc4)CC3)CC2)c(F)c1 ; Cc1nc2ccc(-c3ccnc(N)c3)nc2n1-c1ccc(N2CCN(C(=O)CCN3CCN(Cc4ccc(CNc5cccc6c5CN(C5CCC(=O)NC5=O)C6=O)cc4)CC3)CC2)c(F)c1 ; Cc1nc2ccc(-c3ccnc(N)c3)nc2n1-c1ccc(N2CCN(CCOCCOCCOCCOCCNc3cc4c(cc3F)C(=O)N(C3CCC(=O)NC3=O)C4=O)CC2)c(F)c1 ; Cc1nc2ccc(-c3ccnc(N)c3)nc2n1-c1ccc(N2CCN(C(=O)CCCN3CCN(Cc4ccc(CNc5cccc6c5CN(C5CCC(=O)NC5=O)C6=O)cc4)CC3)CC2)c(F)c1]

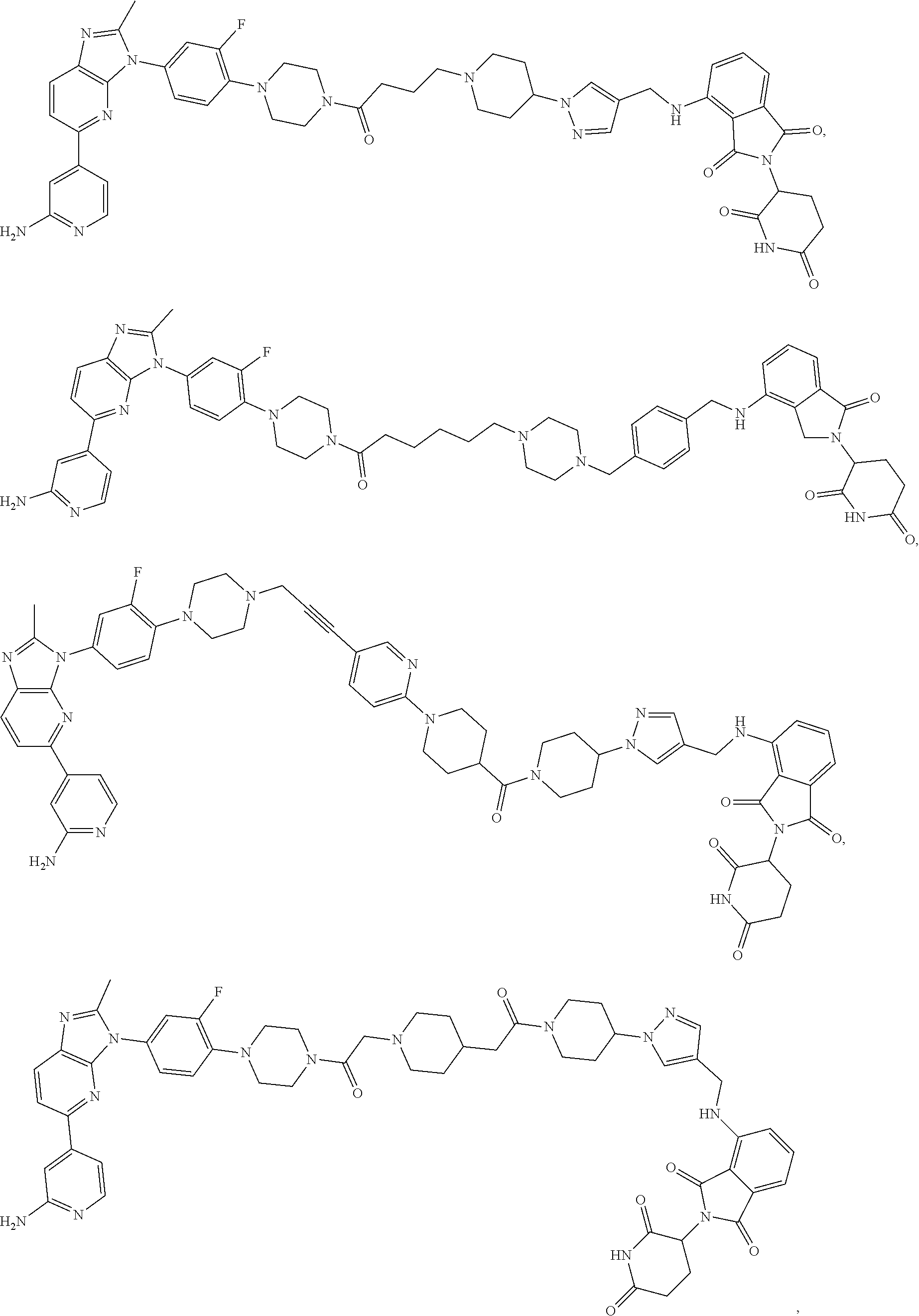

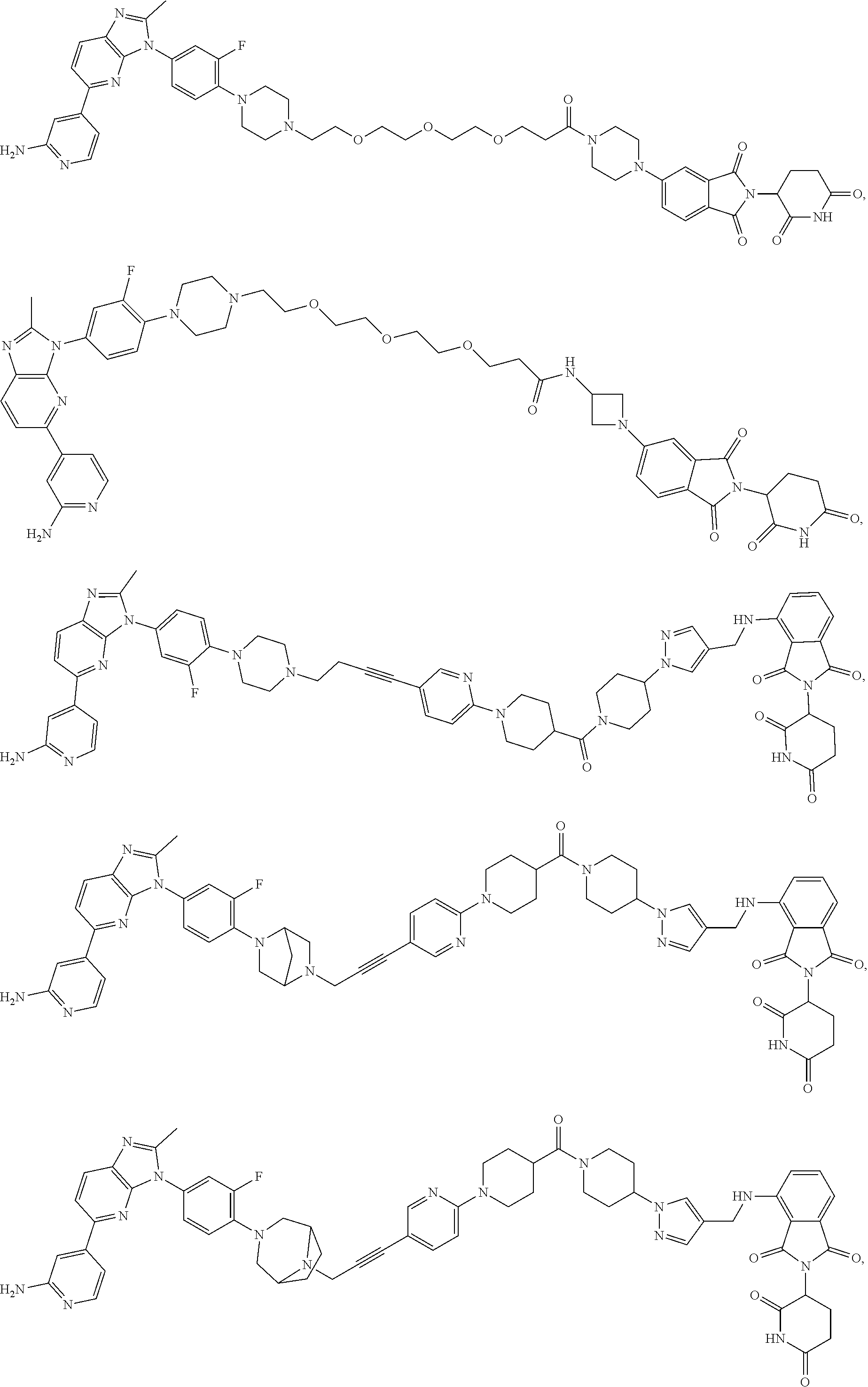

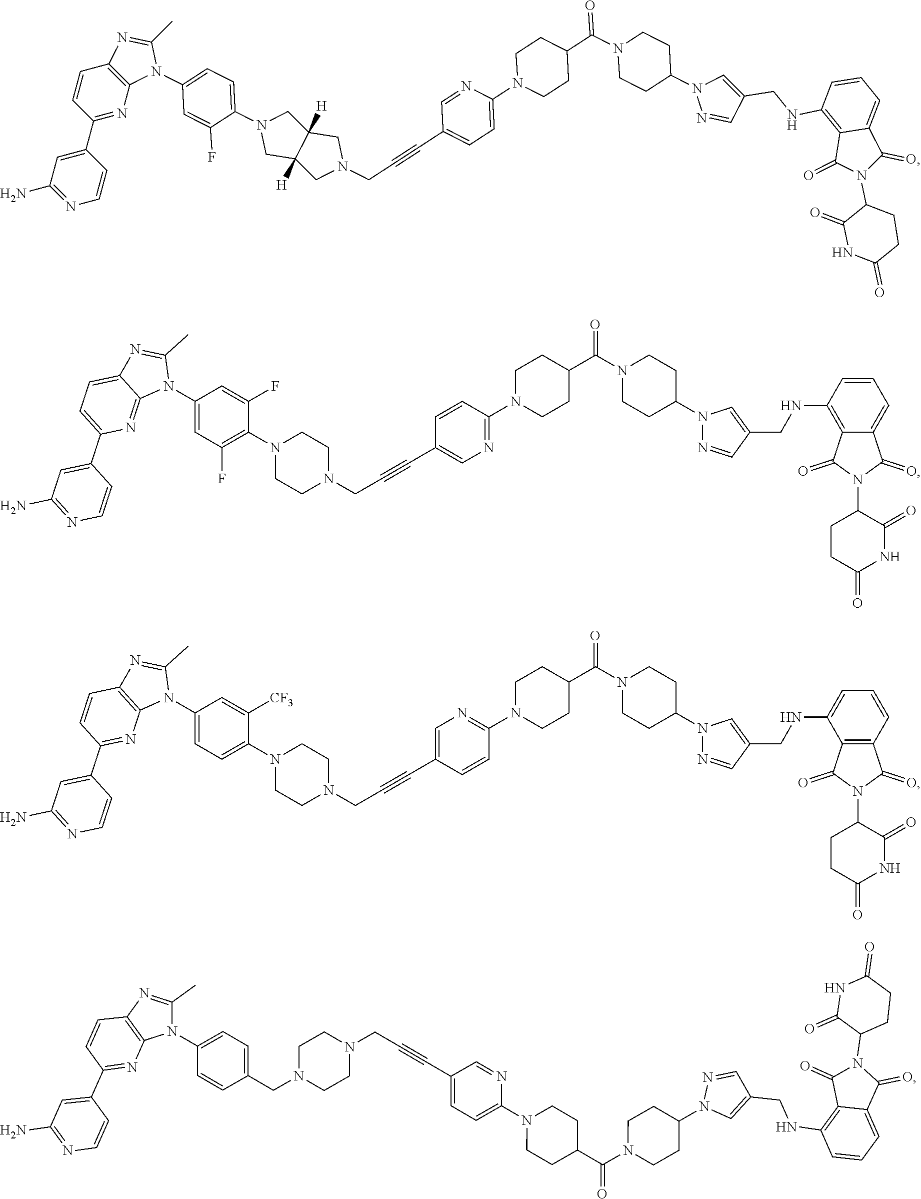

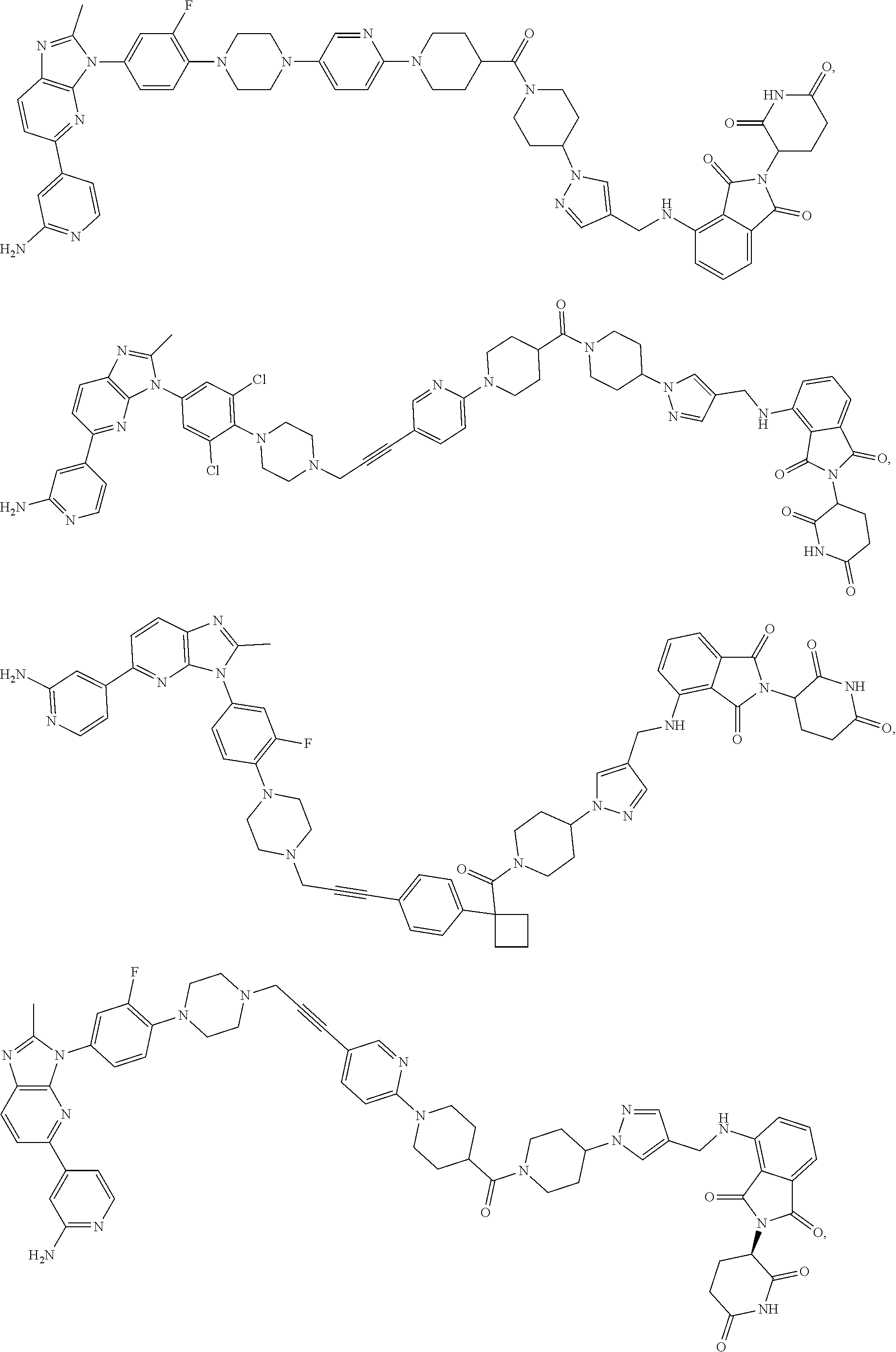

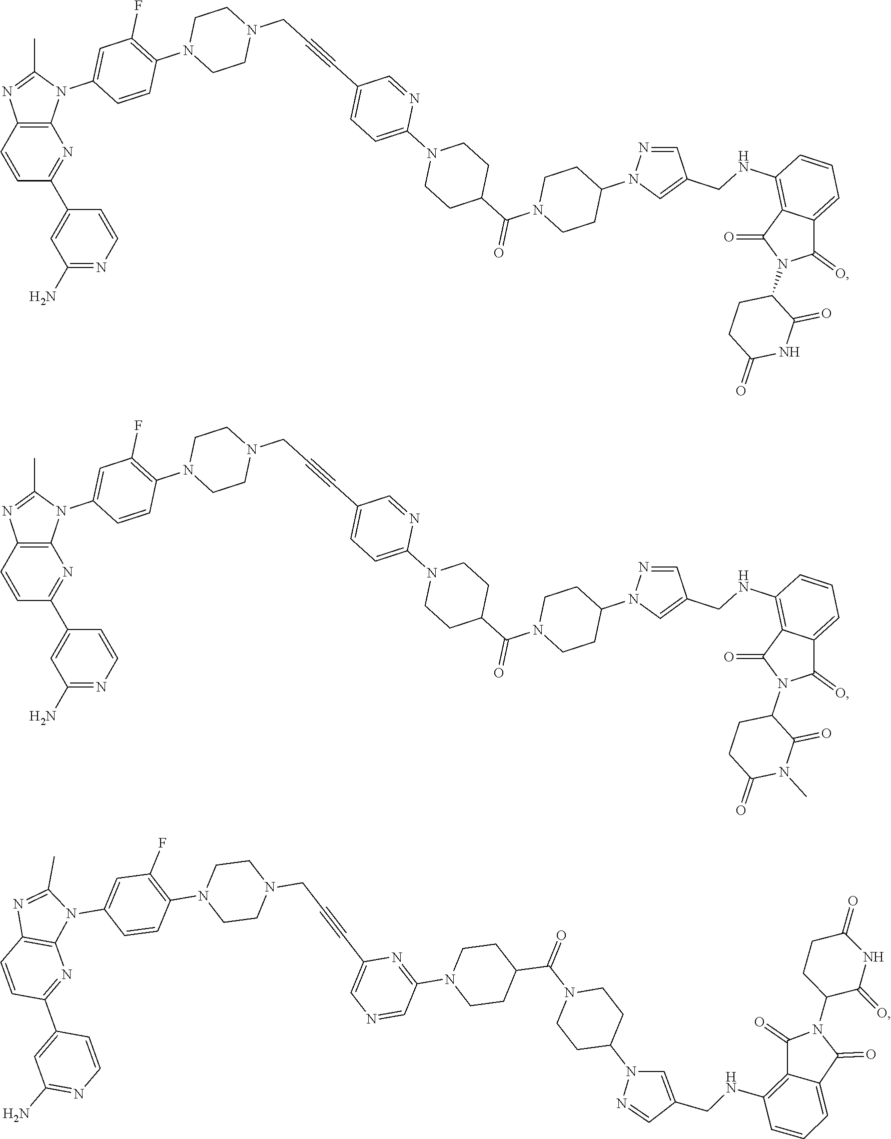 [SMILES: Cc1nc2ccc(-c3ccnc(N)c3)nc2n1-c1ccc(N2CCN(CC#Cc3ccc(N4CCC(C(=O)N5CCC(n6cc(CNc7cccc8c7C(=O)N([C@H]7CCC(=O)NC7=O)C8=O)cn6)CC5)CC4)nc3)CC2)c(F)c1 ; Cc1nc2ccc(-c3ccnc(N)c3)nc2n1-c1ccc(N2CCN(CC#Cc3ccc(N4CCC(C(=O)N5CCC(n6cc(CNc7cccc8c7C(=O)N(C7CCC(=O)N(C)C7=O)C8=O)cn6)CC5)CC4)nc3)CC2)c(F)c1 ; Cc1nc2ccc(-c3ccnc(N)c3)nc2n1-c1ccc(N2CCN(CC#Cc3cncc(N4CCC(C(=O)N5CCC(n6cc(CNc7cccc8c7C(=O)N(C7CCC(=O)NC7=O)C8=O)cn6)CC5)CC4)n3)CC2)c(F)c1]

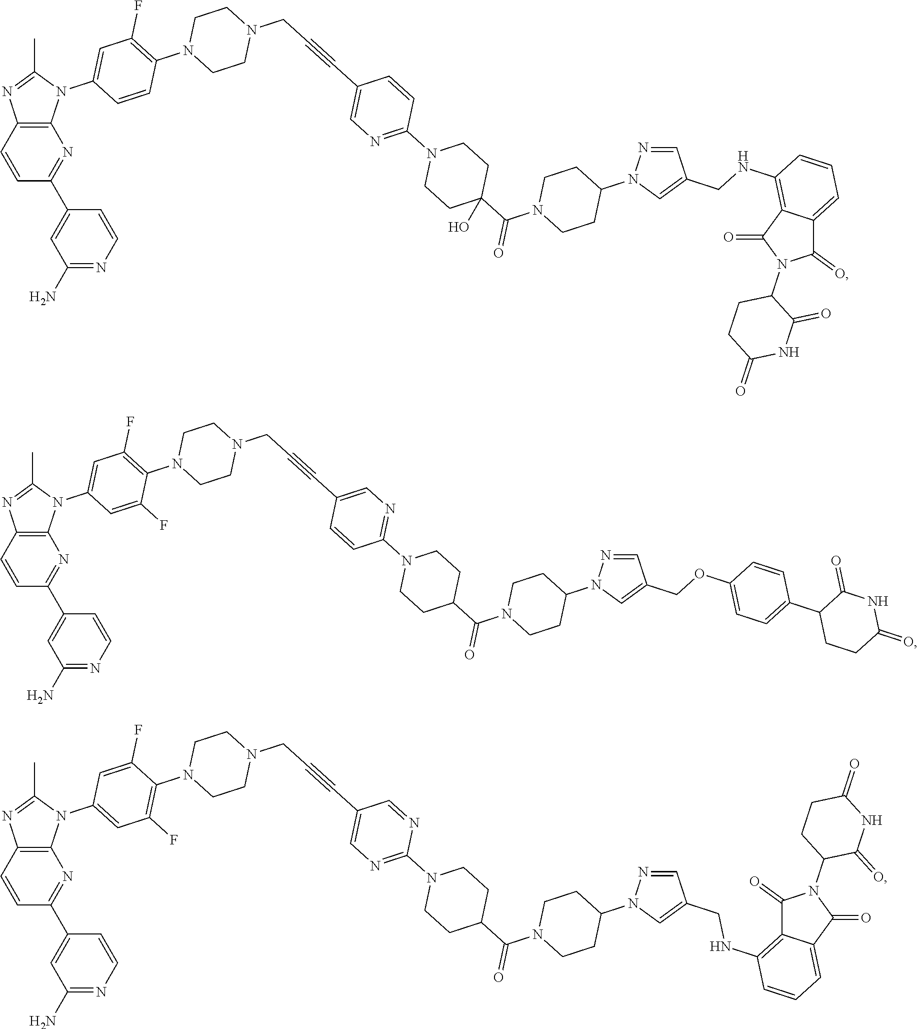 [SMILES: Cc1nc2ccc(-c3ccnc(N)c3)nc2n1-c1ccc(N2CCN(CC#Cc3ccc(N4CCC(O)(C(=O)N5CCC(n6cc(CNc7cccc8c7C(=O)N(C7CCC(=O)NC7=O)C8=O)cn6)CC5)CC4)nc3)CC2)c(F)c1 ; Cc1nc2ccc(-c3ccnc(N)c3)nc2n1-c1cc(F)c(N2CCN(CC#Cc3ccc(N4CCC(C(=O)N5CCC(n6cc(COc7ccc(C8CCC(=O)NC8=O)cc7)cn6)CC5)CC4)nc3)CC2)c(F)c1 ; Cc1nc2ccc(-c3ccnc(N)c3)nc2n1-c1cc(F)c(N2CCN(CC#Cc3cnc(N4CCC(C(=O)N5CCC(n6cc(CNc7cccc8c7C(=O)N(C7CCC(=O)NC7=O)C8=O)cn6)CC5)CC4)nc3)CC2)c(F)c1]

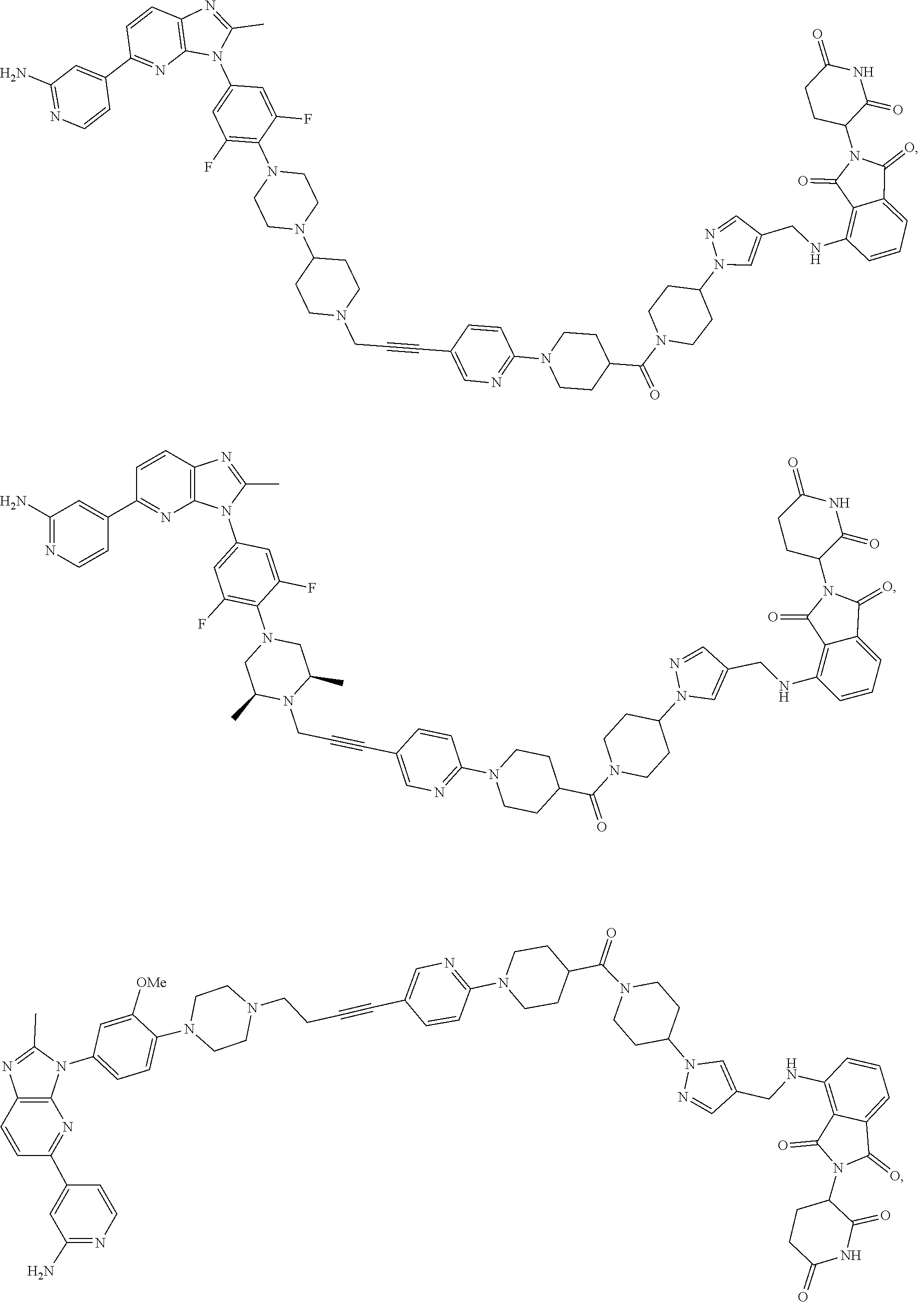 [SMILES: Cc1nc2ccc(-c3ccnc(N)c3)nc2n1-c1cc(F)c(N2CCN(C3CCN(CC#Cc4ccc(N5CCC(C(=O)N6CCC(n7cc(CNc8cccc9c8C(=O)N(C8CCC(=O)NC8=O)C9=O)cn7)CC6)CC5)nc4)CC3)CC2)c(F)c1 ; Cc1nc2ccc(-c3ccnc(N)c3)nc2n1-c1cc(F)c(N2C[C@@H](C)N(CC#Cc3ccc(N4CCC(C(=O)N5CCC(n6cc(CNc7cccc8c7C(=O)N(C7CCC(=O)NC7=O)C8=O)cn6)CC5)CC4)nc3)[C@@H](C)C2)c(F)c1 ; COc1cc(-n2c(C)nc3ccc(-c4ccnc(N)c4)nc32)ccc1N1CCN(CCC#Cc2ccc(N3CCC(C(=O)N4CCC(n5cc(CNc6cccc7c6C(=O)N(C6CCC(=O)NC6=O)C7=O)cn5)CC4)CC3)nc2)CC1]

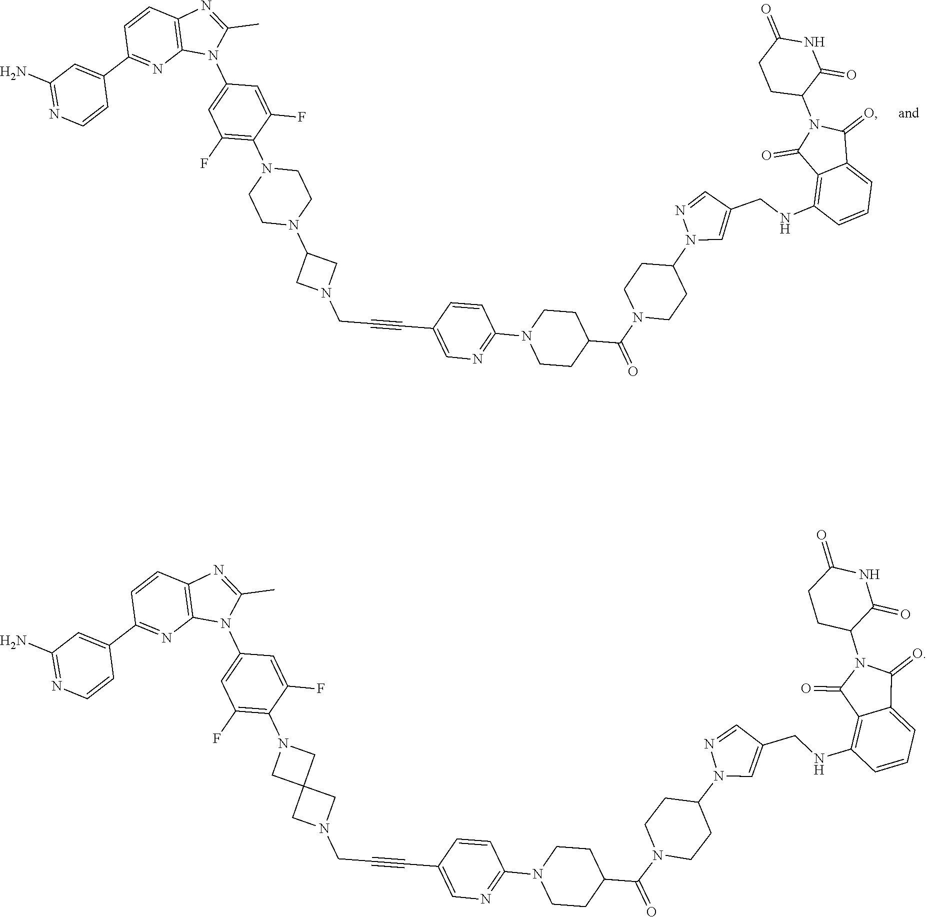 [SMILES: Cc1nc2ccc(-c3ccnc(N)c3)nc2n1-c1cc(F)c(N2CCN(C3CN(CC#Cc4ccc(N5CCC(C(=O)N6CCC(n7cc(CNc8cccc9c8C(=O)N(C8CCC(=O)NC8=O)C9=O)cn7)CC6)CC5)nc4)C3)CC2)c(F)c1 ; "and" ; Cc1nc2ccc(-c3ccnc(N)c3)nc2n1-c1cc(F)c(N2CC3(CN(CC#Cc4ccc(N5CCC(C(=O)N6CCC(n7cc(CNc8cccc9c8C(=O)N(C8CCC(=O)NC8=O)C9=O)cn7)CC6)CC5)nc4)C3)C2)c(F)c1]

Without being bound by any particular theory, it is hypothesized that due at least in part to the proximity of the one or more kinases selected from DYRK1A, DYRK1B, DYRK2, DYRK3, CLK1, CLK2, CLK3, CLK4, and HASPIN and the E3 ubiquitin ligase, the one or more kinases is ubiquitinated by the ubiquitin ligase and degraded.

The von Hippel-Lindau (VHL) tumor suppressor comprises the substrate recognition subunit/E3 ligase complex VCB, which includes elongins B and C, and a complex including Cullin-2 and Rbx1. The primary substrate of VHL is Hypoxia Inducible Factor 1α (HIF-1α), a transcription factor that upregulates genes such as the pro-angiogenic growth factor VEGF and the red blood cell inducing cytokine erythropoietin in response to low oxygen levels. We generated the first small molecule ligands of Von Hippel Lindau (VHL) to the substrate recognition subunit of the E3 ligase, VCB, an important target in cancer, chronic anemia and ischemia, and obtained crystal structures confirming that the compound mimics the binding mode of the transcription factor HIF-1α, the major substrate of VHL.

Inhibitors of Apoptosis Protein (IAPs) are guardian ubiquitin ligases that keep classic pro-apoptotic proteins in check, and regulates not only caspases and apoptosis, but also modulates inflammatory signaling and immunity, copper homeostasis, mitogenic kinase signaling, cell proliferation, as well as cell invasion and metastasis. IAPs act as a direct caspase inhibitor, and directly bind to the active site pocket of CASP3 and CASP7 and obstruct substrate entry. IAPS also inactivate CASP9 by keeping it in a monomeric, inactive state. IAP acts as an E3 ubiquitin-protein ligase regulating NF-kappa-B signaling and the target proteins for its E3 ubiquitin-protein ligase activity include: RIPK1, CASP3, CASP7, CASP8, CASP9, MAP3K2/MEKK2, DIABLO/SMAC, AIFM1, CCS and BIRC5/survivin. Ubiquitination of CCS leads to enhancement of its chaperone activity toward its physiologic target, SOD1, rather than proteasomal degradation. Ubiquitinion of MAP3K2/MEKK2 and AIFM1 does not lead to proteasomal degradation. IAP plays a role in copper homeostasis by ubiquitinating COMMD1 and promoting its proteasomal degradation, and can also function as E3 ubiquitin-protein ligase of the NEDD8 conjugation pathway, targeting effector caspases for neddylation and inactivation. IAP regulates the BMP signaling pathway and the SMAD and MAP3K7/TAK1 dependent pathways leading to NF-kappa-B and JNK activation.

IAPs are an important regulator of innate immune signaling via regulation of Nodlike receptors (NLRs), and protects cells from spontaneous formation of the ripoptosome, a large multi-protein complex that has the capability to kill cancer cells in a caspase-dependent and caspase-independent manner. Suppresses ripoptosome formation by ubiquitinating RIPK1 and CASP8. Acts as a positive regulator of Wnt signaling and ubiquitinates TLE1, TLE2, TLE3, TLE4 and AES. Ubiquitination of TLE3 results in inhibition of its interaction with TCF7L2/TCF4 thereby allowing efficient recruitment and binding of the transcriptional coactivator beta-catenin to TCF7L2/TCF4 that is required to initiate a Wnt-specific transcriptional program. Inhibitors of the IAP, which are useful in making compounds as described herein, are known in the art.

Mouse double minute 2 homolog (MDM2) also known as E3 ubiquitin-protein ligase Mdm2 is a protein that in humans is encoded by the MDM2 gene. Mdm2 is an important negative regulator of the p53 tumor suppressor. Mdm2 protein functions both as an E3 ubiquitin ligase that recognizes the N-terminal trans-activation domain (TAD) of the p53 tumor suppressor and an inhibitor of p53 transcriptional activation. Inhibitors of the MDM2-p53 interaction, which are useful in making compounds as described herein, include the cis-imidazoline analog nutlin.

Cereblon is a protein that in humans is encoded by the CRBN gene. CRBN orthologs are highly conserved from plants to humans, which underscores its physiological importance. Cereblon forms an E3 ubiquitin ligase complex with damaged DNA binding protein 1 (DDB1), Cullin-4A (CUL4A), and regulator of cullins 1 (ROC1). This complex ubiquitinates a number of other proteins. Through a mechanism which has not been completely elucidated, cereblon ubquitination of target proteins results in increased levels of fibroblast growth factor 8 (FGF8) and fibroblast growth factor 10 (FGF10). FGF8 in turn regulates a number of developmental processes, such as limb and auditory vesicle formation. The net result is that this ubiquitin ligase complex is important for limb outgrowth in embryos. In the absence of cereblon, DDB1 forms a complex with DDB2 that functions as a DNA damage-binding protein.

Thalidomide, which has been approved for the treatment of a number of immunological indications, has also been approved for the treatment of certain neoplastic diseases, including multiple myeloma. In addition to multiple myeloma, thalidomide and several of its analogs are also currently under investigation for use in treating a variety of other types of cancer. While the precise mechanism of thalidomide's anti-tumor activity is still emerging, it is known to inhibit angiogenesis. Recent literature discussing the biology of the imides includes Lu et al Science 343, 305 (2014) and Krönke et al Science 343, 301 (2014).

Significantly, thalidomide and its analogs e.g. pomolinamiode and lenalinomide, are known to bind cereblon. These agents bind to cereblon, altering the specificity of the complex to induce the ubiquitination and degradation of Ikaros (IKZF1) and Aiolos (IKZF3), transcription factors essential for multiple myeloma growth. Indeed, higher expression of cereblon has been linked to an increase in efficacy of imide drugs in the treatment of multiple myeloma. Therefore, thalidomide and its analogs are useful cereblon binding moieties for use in making compounds as described herein.

The present description includes, where applicable, the compositions comprising the pharmaceutically acceptable salts, in particular, acid or base addition salts of compounds of the present invention.

The term “pharmaceutically acceptable salt” is used throughout the specification to describe, where applicable, a salt form of one or more of the compounds described herein which are presented to increase the solubility of the compound in the gastic juices of the patient's gastrointestinal tract in order to promote dissolution and the bioavailability of the compounds. Pharmaceutically acceptable salts include those derived from pharmaceutically acceptable inorganic or organic bases and acids, where applicable. Suitable salts include those derived from alkali metals such as potassium and sodium, alkaline earth metals such as calcium, magnesium and ammonium salts, among numerous other acids and bases well known in the pharmaceutical art. Sodium and potassium salts are particularly preferred as neutralization salts of the phosphates according to the present invention.

The acids which are used to prepare the pharmaceutically acceptable acid addition salts of the aforementioned base compounds useful in this invention are those which form non-toxic acid addition salts, i.e., salts containing pharmacologically acceptable anions, such as the hydrochloride, hydrobromide, hydroiodide, nitrate, sulfate, bisulfate, phosphate, acid phosphate, acetate, lactate, citrate, acid citrate, tartrate, bitartrate, succinate, maleate, fumarate, gluconate, saccharate, benzoate, methanesulfonate, ethanesulfonate, benzenesulfonate, p-toluenesulfonate and pamoate [i.e., 1,1′-methylene-bis-(2-hydroxy-3 naphthoate)] salts, among numerous others. Pharmaceutically acceptable base addition salts may also be used to produce pharmaceutically acceptable salt forms of the compounds or derivatives according to the present invention. The chemical bases that may be used as reagents to prepare pharmaceutically acceptable base salts of the present compounds that are acidic in nature are those that form non-toxic base salts with such compounds. Such non-toxic base salts include, but are not limited to those derived from such pharmacologically acceptable cations such as alkali metal cations (eg., potassium and sodium) and alkaline earth metal cations (eg, calcium, zinc and magnesium), ammonium or water-soluble amine addition salts such as N-methylglucamine-(meglumine), and the lower alkanolammonium and other base salts of pharmaceutically acceptable organic amines, among others.

In another aspect, the description provides compositions comprising compounds as described herein, including salts thereof, and a pharmaceutically acceptable carrier. In certain embodiments, the compositions are therapeutic or pharmaceutical compositions comprising an effective amount of a compound as described herein and a pharmaceutically acceptable carrier.

The amount of compound in a pharmaceutical composition of the instant invention that may be combined with the carrier materials to produce a single dosage form will vary depending upon the host and disease treated, the particular mode of administration. Generally, an amount between 0.1 mg/kg and 1000 mg/kg body weight/day of active ingredients is administered dependent upon potency of the agent. Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects. The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

The compositions of the present invention may be formulated in a conventional manner using one or more pharmaceutically acceptable carriers and may also be administered in controlled-release formulations. Pharmaceutically acceptable carriers that may be used in these pharmaceutical compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as prolamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The active compound is included in the pharmaceutically acceptable carrier or diluent in an amount sufficient to deliver to a patient a therapeutically effective amount for the desired indication, without causing serious toxic effects in the patient treated. A preferred dose of the active compound for all of the herein-mentioned conditions is in the range from about 10 ng/kg to 300 mg/kg, preferably 0.1 to 100 mg/kg per day, more generally 0.5 to about 25 mg per kilogram body weight of the recipient/patient per day. A typical topical dosage will range from 0.01-5% wt/wt in a suitable carrier.

The compound is conveniently administered in any suitable unit dosage form, including but not limited to one containing less than 1 mg, 1 mg to 3000 mg, preferably 5 to 500 mg of active ingredient per unit dosage form. An oral dosage of about 25-250 mg is often convenient.

The active ingredient is preferably administered to achieve peak plasma concentrations of the active compound of about 0.00001-30 mM, preferably about 0.1-30 μM. This may be achieved, for example, by the intravenous injection of a solution or formulation of the active ingredient, optionally in saline, or an aqueous medium or administered as a bolus of the active ingredient. Oral administration is also appropriate to generate effective plasma concentrations of active agent.

The concentration of active compound in the drug composition will depend on absorption, distribution, inactivation, and excretion rates of the drug as well as other factors known to those of skill in the art. It is to be noted that dosage values will also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed composition. The active ingredient may be administered at once, or may be divided into a number of smaller doses to be administered at varying intervals of time.

If administered intravenously, preferred carriers are physiological saline or phosphate buffered saline (PBS).

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art.

Liposomal suspensions may also be pharmaceutically acceptable carriers. These may be prepared according to methods known to those skilled in the art, for example, as described in U.S. Pat. No. 4,522,811 (which is incorporated herein by reference in its entirety). For example, liposome formulations may be prepared by dissolving appropriate lipid(s) (such as stearoyl phosphatidyl ethanolamine, stearoyl phosphatidyl choline, arachadoyl phosphatidyl choline, and cholesterol) in an inorganic solvent that is then evaporated, leaving behind a thin film of dried lipid on the surface of the container. An aqueous solution of the active compound are then introduced into the container. The container is then swirled by hand to free lipid material from the sides of the container and to disperse lipid aggregates, thereby forming the liposomal suspension.

In any of the aspects or embodiments described herein, the therapeutic compositions comprising compounds described herein can be in any suitable dosage form configured to be delivered by any suitable route. For example, the compounds can be administered by any appropriate route, for example, orally, parenterally, intravenously, intradermally, subcutaneously, or topically, including transdermally, in liquid, cream, gel, or solid form, rectally, nasally, buccally, vaginally or via an implanted reservoir or by aerosol form.

The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously.

The compounds as described herein may be administered in single or divided doses by the oral, parenteral or topical routes. Administration of the active compound may range from continuous (intravenous drip) to several oral administrations per day (for example, Q.I.D.) and may include oral, topical, parenteral, intramuscular, intravenous, sub-cutaneous, transdermal (which may include a penetration enhancement agent), buccal, sublingual and suppository administration, among other routes of administration. Enteric coated oral tablets may also be used to enhance bioavailability of the compounds from an oral route of administration. The most effective dosage form will depend upon the pharmacokinetics of the particular agent chosen as well as the severity of disease in the patient.

Administration of compounds as sprays, mists, or aerosols for intra-nasal, intra-tracheal or pulmonary administration may also be used. Compounds as described herein may be administered in immediate release, intermediate release or sustained or controlled release forms. Sustained or controlled release forms are preferably administered orally, but also in suppository and transdermal or other topical forms. Intramuscular injections in liposomal form may also be used to control or sustain the release of compound at an injection site.

Sterile injectable forms of the compositions as described herein may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as Ph. Helv or similar alcohol.

The pharmaceutical compositions as described herein may be orally administered in any orally acceptable dosage form including, but not limited to, capsules, tablets, aqueous suspensions or solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions are required for oral use, the active ingredient is combined with emulsifying and suspending agents. If desired, certain sweetening, flavoring or coloring agents may also be added. Oral compositions will generally include an inert diluent or an edible carrier. They may be enclosed in gelatin capsules or compressed into tablets. For the purpose of oral therapeutic administration, the active compound or its prodrug derivative can be incorporated with excipients and used in the form of tablets, troches, or capsules. Pharmaceutically compatible binding agents, and/or adjuvant materials are included as part of the composition.

The tablets, pills, capsules, troches and the like can contain any of the following ingredients, or compounds of a similar nature: a binder such as microcrystalline cellulose, gum tragacanth or gelatin; an excipient such as starch or lactose, a dispersing agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. When the dosage unit form is a capsule, it can contain, in addition to material of the above type, a liquid carrier such as a fatty oil. In addition, dosage unit forms can contain various other materials which modify the physical form of the dosage unit, for example, coatings of sugar, shellac, or enteric agents.

The active compound or pharmaceutically acceptable salt thereof can be administered as a component of an elixir, suspension, syrup, wafer, chewing gum or the like. A syrup may contain, in addition to the active compounds, sucrose as a sweetening agent and certain preservatives, dyes and colorings and flavors.

Alternatively, the pharmaceutical compositions as described herein may be administered in the form of suppositories for rectal administration. These can be prepared by mixing the agent with a suitable non-irritating excipient, which is solid at room temperature but liquid at rectal temperature and therefore will melt in the rectum to release the drug. Such materials include cocoa butter, beeswax and polyethylene glycols.

The pharmaceutical compositions of this invention may also be administered topically. Suitable topical formulations are readily prepared for each of these areas or organs. Topical application for the lower intestinal tract can be effected in a rectal suppository formulation (see above) or in a suitable enema formulation. Topically-acceptable transdermal patches may also be used. For topical applications, the pharmaceutical compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. In certain preferred aspects of the invention, the compounds may be coated onto a stent which is to be surgically implanted into a patient in order to inhibit or reduce the likelihood of occlusion occurring in the stent in the patient.

Alternatively, the pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, the pharmaceutical compositions may be formulated as micronized suspensions in isotonic, pH adjusted sterile saline, or, preferably, as solutions in isotonic, pH adjusted sterile saline, either with our without a preservative such as benzylalkonium chloride. Alternatively, for ophthalmic uses, the pharmaceutical compositions may be formulated in an ointment such as petrolatum.

The pharmaceutical compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Solutions or suspensions used for parenteral, intradermal, subcutaneous, or topical application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parental preparation can be enclosed in ampoules, disposable syringes or multiple dose vials made of glass or plastic.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease or condition being treated.

A patient or subject in need of therapy using compounds as described herein can be treated by administering to the patient (subject) an effective amount of the compound including pharmaceutically acceptable salts, solvates or polymorphs, thereof optionally in a pharmaceutically acceptable carrier or diluent, either alone, or in combination with other known agents.

Disease states of conditions which may be treated using compounds or compositions according to the present description include, but not limited to, for example, Alzheimer's disease and cancer. In certain embodiments, the therapeutic or pharmaceutical compositions comprise an effective amount of an additional biologically or bioactive active agent, e.g., an agent effective for the treatment of cancer, that is co-administered.

The term “coadministration” or “combination therapy” shall mean that at least two compounds or compositions are administered to the patient at the same time, such that effective amounts or concentrations of each of the two or more compounds may be found in the patient at a given point in time. Although compounds according to the present invention may be co-administered to a patient at the same time, the term embraces both administration of two or more agents at the same time or at different times, provided that effective concentrations of all coadministered compounds or compositions are found in the subject at a given time. In certain preferred aspects of the present invention, one or more of the present compounds described above, are coadministered in combination with at least one additional bioactive agent, especially including an anticancer agent. In particularly preferred aspects of the invention, the co-administration of compounds results in synergistic therapeutic, including anticancer therapy.

In another aspect, the description provides a composition comprising an effective amount of two or more of the PROTAC compounds as described herein, and a pharmaceutically acceptable carrier. In certain embodiments, the composition further comprises an effective or synergistic amount of another bioactive agent that is not a PROTAC compound.

Pharmaceutical compositions comprising combinations of an effective amount of at least one bifunctional compound according to the present invention, and one or more of the compounds otherwise described herein, all in effective amounts, in combination with a pharmaceutically effective amount of a carrier, additive or excipient, represents a further aspect of the present invention.

The term “bioactive agent” is used to describe an agent, other than the PROTAC compounds described herein, which is used in combination with the present compounds as an agent with biological activity to assist in effecting an intended therapy, inhibition and/or prevention/prophylaxis for which the present compounds are used. Preferred bioactive agents for use herein include those agents which have pharmacological activity similar to that for which the present compounds are used or administered and include for example, anti-cancer agents.

The term “additional anti-cancer agent” is used to describe an anti-cancer agent, which may be combined with PROTAC compounds according to the present description to treat cancer.

These agents include, for example, everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, a FLT-3 inhibitor, an androgen receptor inhibitor, a VEGFR inhibitor, an EGFR TK inhibitor, an aurora kinase inhibitor, a PIK-1 modulator, a Bcl-2 inhibitor, an HDAC inhibitor, a c-MET inhibitor, a PARP inhibitor, a Cdk inhibitor, an EGFR TK inhibitor, an IGFR-TK inhibitor, an anti-HGF antibody, a PI3 kinase inhibitors, an AKT inhibitor, a JAK/STAT inhibitor, a checkpoint-1 or 2 inhibitor, a focal adhesion kinase inhibitor, a Map kinase kinase (mek) inhibitor, a VEGF trap antibody, pemetrexed, erlotinib, dasatanib, nilotinib, decatanib, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, INO 1001, IPdR1 KRX-0402, lucanthone, LY317615, neuradiab, vitespan, Rta 744, Sdx 102, talampanel, atrasentan, Xr 311, romidepsin, ADS-100380, sunitinib, 5-fluorouracil, vorinostat, etoposide, gemcitabine, doxorubicin, liposomal doxorubicin, 5′-deoxy-5-fluorouridine, vincristine, temozolomide, ZK-304709, seliciclib; PD0325901, AZD-6244, capecitabine, L-Glutamic acid, N-[4-[2-(2-amino-4,7-dihydro-4-oxo-1H-pyrrolo[2,3-d]pyrimidin-5-yl)ethyl]benzoyl]-, disodium salt, heptahydrate, camptothecin, PEG-labeled irinotecan, tamoxifen, toremifene citrate, anastrazole, exemestane, letrozole, DES (diethylstilbestrol), estradiol, estrogen, conjugated estrogen, bevacizumab, IMC-1C11, CHIR-258); 3-[5-(methylsulfonylpiperadinemethyl)-indolylj-quinolone, vatalanib, AG-013736, AVE-0005, the acetate salt of [D-Ser (Bu t) 6,Azgly 10](pyro-Glu-His-Trp-Ser-Tyr-D-Ser (Bu t)-Leu-Arg-Pro-Azgly-NH2 acetate [C₅₉H₈₄N₁₈O₁₄-(C₂H₄O₂)ₓ where x=1 to 2.4], goserelin acetate, leuprolide acetate, triptorelin pamoate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, erbitux, EKB-569, PKI-166, GW-572016, Ionafarnib, BMS-214662, tipifarnib; amifostine, NVP-LAQ824, suberoyl analide hydroxamic acid, valproic acid, trichostatin A, FK-228, SU11248, sorafenib, KRN951, aminoglutethimide, arnsacrine, anagrelide, L-asparaginase, Bacillus Calmette-Guerin (BCG) vaccine, adriamycin, bleomycin, buserelin, busulfan, carboplatin, carmustine, chlorambucil, cisplatin, cladribine, clodronate, cyproterone, cytarabine, dacarbazine, dactinomycin, daunorubicin, diethylstilbestrol, epirubicin, fludarabine, fludrocortisone, fluoxymesterone, flutamide, gleevec, gemcitabine, hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mecaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox, gefitinib, bortezimib, paclitaxel, cremophor-free paclitaxel, docetaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, topotecan, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonist, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa, darbepoetin alfa and mixtures thereof.

In another aspect, the disclosure provides methods of modulating protein ubiquitination and degradation in a subject, e.g., a cell, a tissue, mammal, or human patient, the method comprising administering an effective amount of a compound as described herein or a composition comprising an effective amount of the same to a subject, wherein the compound or composition comprising the same is effective in modulating protein ubquitination and degration of the protiein in the subject. In certain embodiments, the protein is one or more kinases selected from: DYRK1A, DYRK1B, DYRK2, DYRK3, CLK1, CLK2, CLK3, CLK4, and HASPIN.

In certain embodiments, the description provides a method for regulating protein activity of one or more kinases selected from DYRK1A, DYRK1B, DYRK2, DYRK3, CLK1, CLK2, CLK3, CLK4, and HASPIN in a patient in need comprising administering to said patient an amount of a compound as described herein to a patient.

In still additional embodiments, the description provides a method of treating a disease state or condition in a patient wherein dysregulated protein activity is responsible for said disease state or condition, said method comprising administering to said patient an effective amount of a compound as described herein to said patient in order to regulate said protein activity in said patient. In certain embodiments, the protein is one or more kinases selected from: DYRK1A, DYRK1B, DYRK2, DYRK3, CLK1, CLK2, CLK3, CLK4, and HASPIN.

The terms “treat”, “treating”, and “treatment”, etc., as used herein, refer to any action providing a benefit to a patient for which the present compounds may be administered, including the treatment of any disease state or condition which is modulated through the protein to which the present compounds bind. Disease states or conditions, including cancer, which may be treated using compounds according to the present invention are set forth hereinabove.

In another aspect, the disclosure provides methods of modulating protein ubiquitination and degradation in a subject, e.g., a cell, a tissue, mammal, or human patient, the method comprising administering an effective amount of a compound as described herein or a composition comprising an effective amount of the same to a subject, wherein the compound or composition comprising the same is effective in modulating protein ubiquitination and degradation of the protein in the subject. In certain embodiments, the protein is one or more of DYRK1A, DYRK1B, DYRK2, DYRK3, CLK1, CLK2, CLK3, CLK4, and HASPIN.

In another aspect, the disclosure provides methods of modulating protein ubiquitination and degradation of one or more of DYRK1A, DYRK1B, DYRK2, DYRK3, CLK1, CLK2, CLK3,

CLK4, and HASPIN in a subject, e.g., a cell, a tissue, mammal, or human patient, the method comprising administering an effective amount of a compound as described herein or a composition comprising an effective amount of the same to a subject, wherein the compound or composition comprising the same is effective in modulating protein ubiquitination and degradation of one or more of DYRK1A, DYRK1B, DYRK2, DYRK3, CLK1, CLK2, CLK3,

CLK4, and HASPIN in the subject.

In another aspect, the disclosure provides methods of treating or ameliorating a symptom of a disease related to one or more of DYRK1A, DYRK1B, DYRK2, DYRK3, CLK1, CLK2, CLK3, CLK4, and HASPIN activity in a subject, e.g., a cell, a tissue, mammal, or human patient, the method comprising administering an effective amount of a compound as described herein or a composition comprising an effective amount of the same to a subject in need thereof, wherein the compound or composition comprising the same is effective in treating or ameliorating a symptom of a disease related to one or more of DYRK1A, DYRK1B, DYRK2, DYRK3, CLK1, CLK2, CLK3, CLK4, and HASPIN activity in the subject. In a preferred embodiment, the subject is a human.

In another aspect, the disclosure provides methods for identifying the effects of the degradation of proteins of interest in a biological system (e.g., one or more of DYRK1A, DYRK1B, DYRK2, DYRK3, CLK1, CLK2, CLK3, CLK4, and HASPIN) using compounds according to the present invention.

In certain embodiments, the present invention provides methods for administering an effective amount of a compound as described herein or a composition comprising an effective amount of the same (alone or with additional agents (e.g., remdesivir)) to a subject (e.g., a human subject) (e.g., a human subject suffering from or at risk of suffering from viral infection) (a human subject suffering from or at risk of suffering from a condition related to a viral infection for purposes of treating, preventing and/or ameliorating the viral infection and/or symptoms of a viral infection (e.g., SARS-COV-2 infection (e.g., COVID-19)). In such embodiments, administration results in suppression of pro-inflammatory cytokine activity (e.g., IL-6 activity) within the subject. In some embodiments, administration results in enhancement of NK cell activity within the subject. In some embodiments, administration results in enhancement of neutrophil activity within the subject. In some embodiments, administration results in inhibition of viral entry into the subject's cells through inhibiting binding of the virus with heparin sulfate proteoglycan within such cells.

In such embodiments, the methods are not limited to treating, preventing and/or ameliorating a particular type or kind of viral infection or symptoms of a particular type or kind of viral infection. In some embodiments, the viral infection is a SARS-COV-2 related viral infection (e.g., COVID-19). In some embodiments, the viral infection is any infection related to influenza, HIV, HIV-1, HIV-2, drug-resistant HIV, Junin virus, Chikungunya virus, Yellow Fever virus, Dengue virus, Pichinde virus, Lassa virus, adenovirus, Measles virus, Punta Toro virus, Respiratory Syncytial virus, Rift Valley virus, RHDV, SARS coronavirus, Tacaribe virus, and

West Nile virus. In some embodiments, the viral infection is associated with any virals having MPTO protease activity and/or expression.

In some embodiments, the one or more symptoms related to viral infection includes, but is not limited to, fever, fatigue, dry cough, myalgias, dyspnea, acute respiratory distress syndrome, and pneumonia.

In another aspect, the description provides kits comprising compounds or compositions as described herein. The kit may be promoted, distributed, or sold as a unit for performing the methods of the present invention. In addition, the kits of the present invention may preferably contain instructions which describe a suitable use. Such kits can be conveniently used, e.g., in clinical settings, to treat patients exhibiting symptoms of, e.g., Alzheimer's disease, down syndrome, diabetes, an autoimmune disease, an inflammatory disorder (e.g., airway inflammation, osteoarthritis (e.g., knee related osteoarthritis)), cancer (e.g., glioblastoma, prostate cancer, metastatic breast cancer, metastatic lung cancer, multiple myeloma, secondary metastatic tumors of the brain, colorectal cancer), a viral infection (e.g., SARS-COV-2 infection (e.g., COVID-19)), and other diseases.

### Examples

The following examples are illustrative, but not limiting, of the compounds, compositions, and methods of the present invention. Other suitable modifications and adaptations of the variety of conditions and parameters normally encountered in clinical therapy and which are obvious to those skilled in the art are within the spirit and scope of the invention.

### Example I

This example provides synthesis and characterization information for compounds of the present invention. Experiments were conducted involving the designing, synthesis and biological evaluation of various Protac compounds described herein capable of inhibiting one or more of DYRK1A, DYRK1B, DYRK2, DYRK3, and CLK1, CLK2, CLK3, CLK4, and HASPIN (see Table 1).

TABLE 1

| Compound ID | Structure |
|---|---|
| 1 | 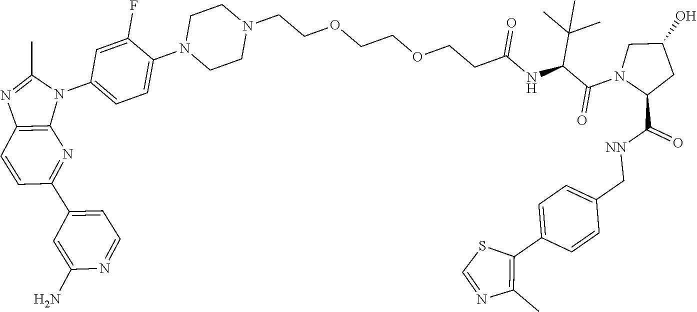 |
| 2 | 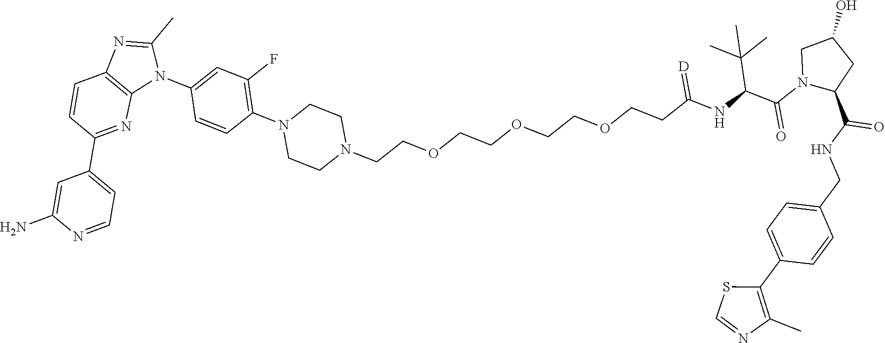 |
| 3 | 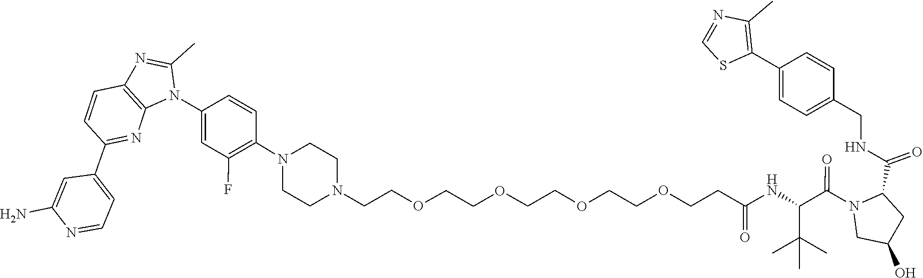 [SMILES: Cc1ncsc1-c1ccc(CNC(=O)[C@@H]2C[C@@H](O)CN2C(=O)[C@@H](NC(=O)CCOCCOCCOCCOCCN2CCN(c3ccc(-n4c(C)nc5ccc(-c6ccnc(N)c6)nc54)cc3F)CC2)C(C)(C)C)cc1] |
| 4 | 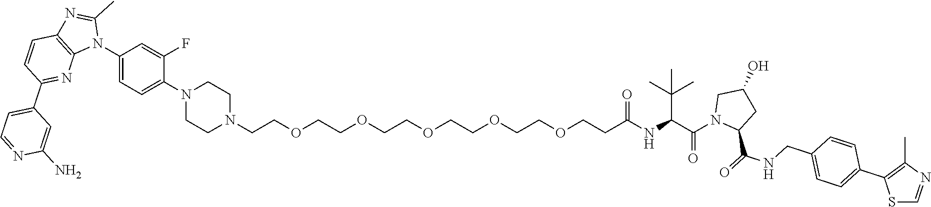 [SMILES: Cc1ncsc1-c1ccc(CNC(=O)[C@@H]2C[C@@H](O)CN2C(=O)[C@@H](NC(=O)CCOCCOCCOCCOCCOCCN2CCN(c3ccc(-n4c(C)nc5ccc(-c6ccnc(N)c6)nc54)cc3F)CC2)C(C)(C)C)cc1] |
| 5 | 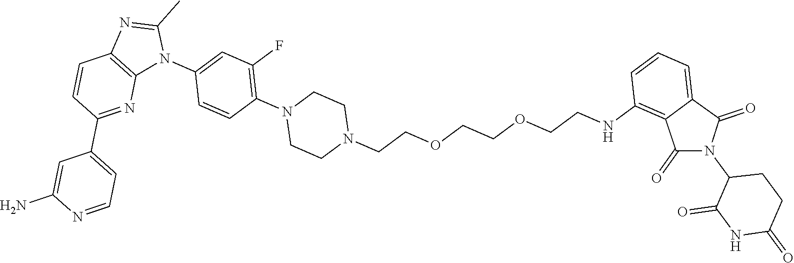 [SMILES: Cc1nc2ccc(-c3ccnc(N)c3)nc2n1-c1ccc(N2CCN(CCOCCOCCNc3cccc4c3C(=O)N(C3CCC(=O)NC3=O)C4=O)CC2)c(F)c1] |
| 6 | 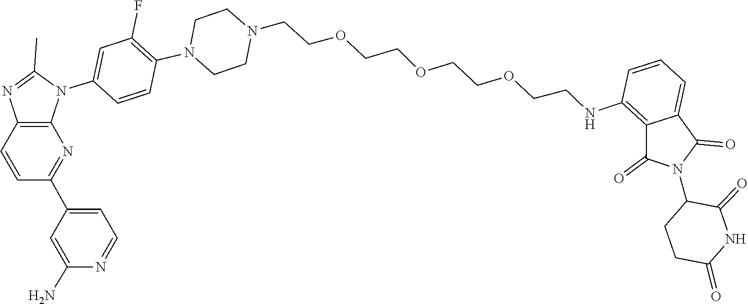 [SMILES: Cc1nc2ccc(-c3ccnc(N)c3)nc2n1-c1ccc(N2CCN(CCOCCOCCOCCNc3cccc4c3C(=O)N(C3CCC(=O)NC3=O)C4=O)CC2)c(F)c1] |
| 7 | 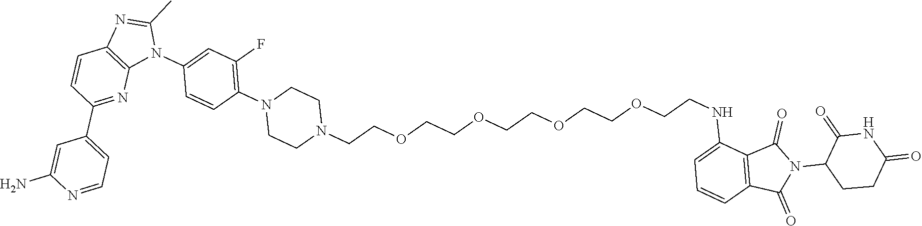 [SMILES: Cc1nc2ccc(-c3ccnc(N)c3)nc2n1-c1ccc(N2CCN(CCOCCOCCOCCOCCNc3cccc4c3C(=O)N(C3CCC(=O)NC3=O)C4=O)CC2)c(F)c1] |
| 8 | 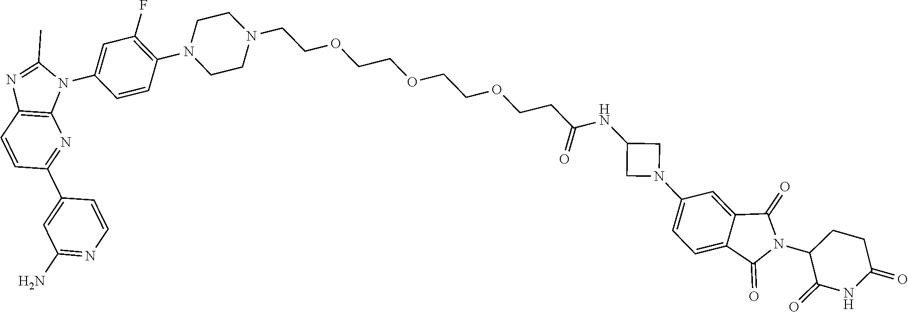 [SMILES: Cc1nc2ccc(-c3ccnc(N)c3)nc2n1-c1ccc(N2CCN(CCOCCOCCOCCC(=O)NC3CN(c4ccc5c(c4)C(=O)N(C4CCC(=O)NC4=O)C5=O)C3)CC2)c(F)c1] |
| 9 | 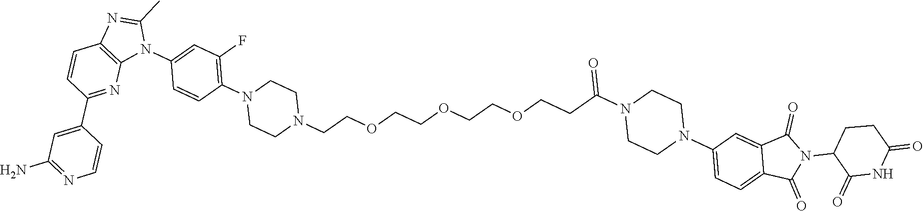 [SMILES: Cc1nc2ccc(-c3ccnc(N)c3)nc2n1-c1ccc(N2CCN(CCOCCOCCOCCC(=O)N3CCN(c4ccc5c(c4)C(=O)N(C4CCC(=O)NC4=O)C5=O)CC3)CC2)c(F)c1] |
| 10 | 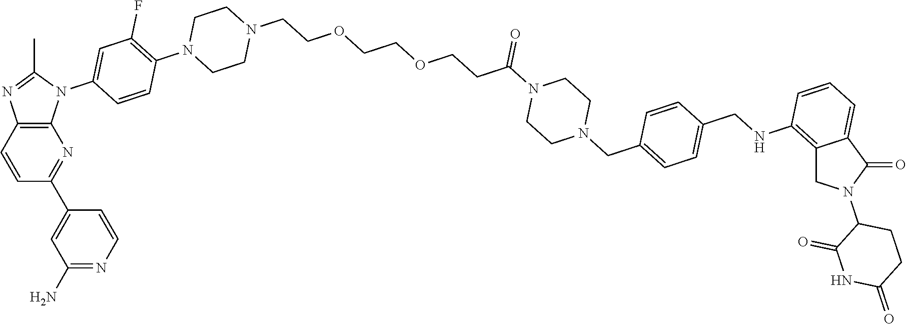 [SMILES: Cc1nc2ccc(-c3ccnc(N)c3)nc2n1-c1ccc(N2CCN(CCOCCOCCC(=O)N3CCN(Cc4ccc(CNc5cccc6c5CN(C5CCC(=O)NC5=O)C6=O)cc4)CC3)CC2)c(F)c1] |
| 11 | 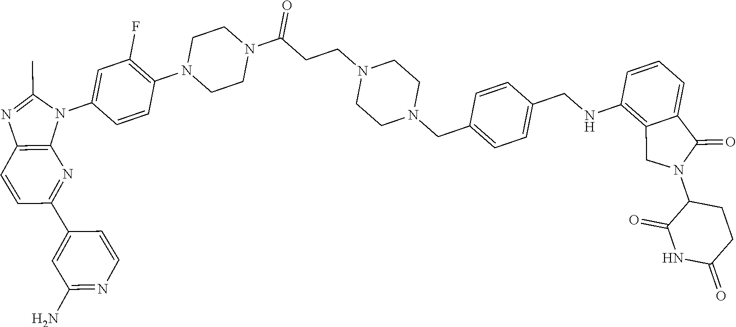 [SMILES: Cc1nc2ccc(-c3ccnc(N)c3)nc2n1-c1ccc(N2CCN(C(=O)CCN3CCN(Cc4ccc(CNc5cccc6c5CN(C5CCC(=O)NC5=O)C6=O)cc4)CC3)CC2)c(F)c1] |
| 12 | 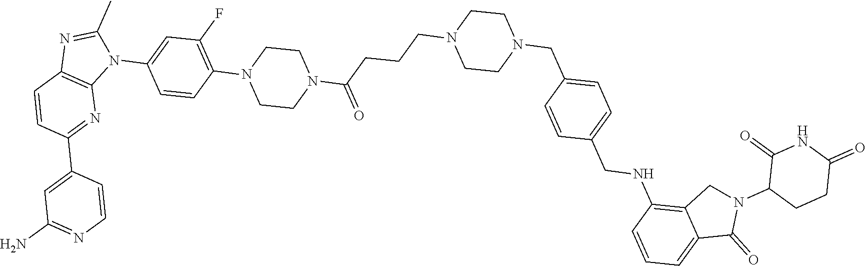 [SMILES: Cc1nc2ccc(-c3ccnc(N)c3)nc2n1-c1ccc(N2CCN(C(=O)CCCN3CCN(Cc4ccc(CNc5cccc6c5CN(C5CCC(=O)NC5=O)C6=O)cc4)CC3)CC2)c(F)c1] |
| 13 | 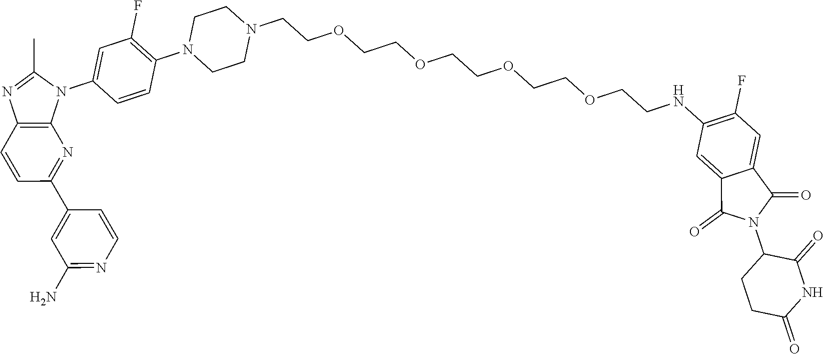 [SMILES: Cc1nc2ccc(-c3ccnc(N)c3)nc2n1-c1ccc(N2CCN(CCOCCOCCOCCOCCNc3cc4c(cc3F)C(=O)N(C3CCC(=O)NC3=O)C4=O)CC2)c(F)c1] |
| 14 | 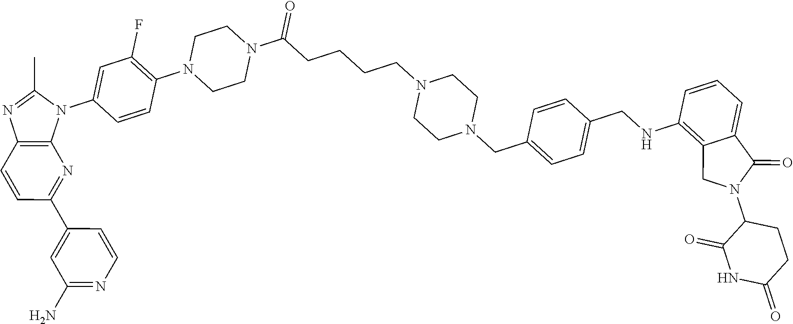 [SMILES: Cc1nc2ccc(-c3ccnc(N)c3)nc2n1-c1ccc(N2CCN(C(=O)CCCCN3CCN(Cc4ccc(CNc5cccc6c5CN(C5CCC(=O)NC5=O)C6=O)cc4)CC3)CC2)c(F)c1] |
| 15 | 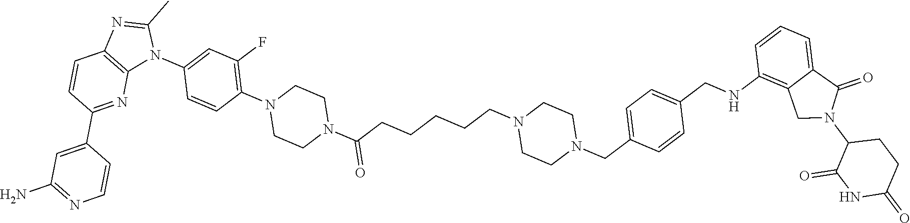 [SMILES: Cc1nc2ccc(-c3ccnc(N)c3)nc2n1-c1ccc(N2CCN(C(=O)CCCCCN3CCN(Cc4ccc(CNc5cccc6c5CN(C5CCC(=O)NC5=O)C6=O)cc4)CC3)CC2)c(F)c1] |
| 16 | 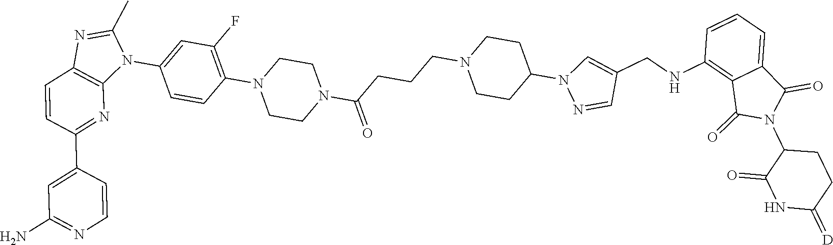 |
| 17 | 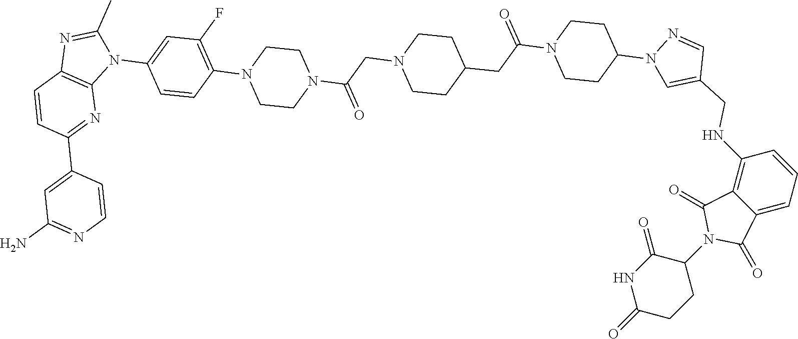 [SMILES: Cc1nc2ccc(-c3ccnc(N)c3)nc2n1-c1ccc(N2CCN(C(=O)CN3CCC(CC(=O)N4CCC(n5cc(CNc6cccc7c6C(=O)N(C6CCC(=O)NC6=O)C7=O)cn5)CC4)CC3)CC2)c(F)c1] |
| 18 | 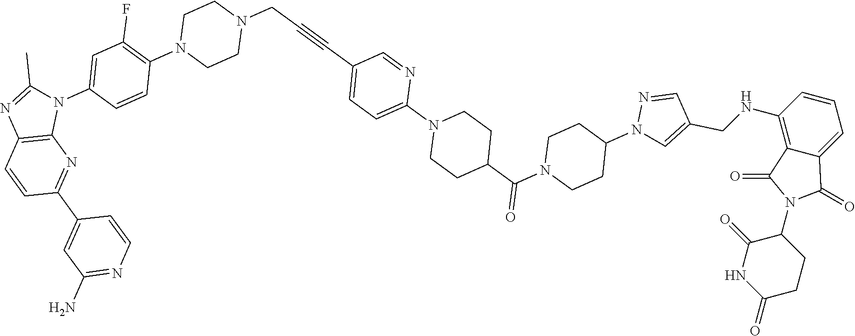 [SMILES: Cc1nc2ccc(-c3ccnc(N)c3)nc2n1-c1ccc(N2CCN(CC#Cc3ccc(N4CCC(C(=O)N5CCC(n6cc(CNc7cccc8c7C(=O)N(C7CCC(=O)NC7=O)C8=O)cn6)CC5)CC4)nc3)CC2)c(F)c1] |
| 19 | 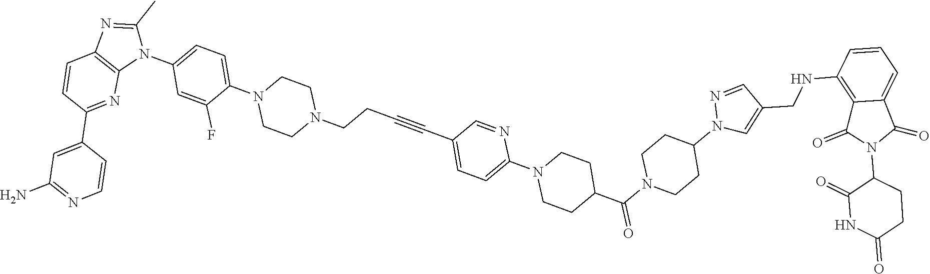 [SMILES: Cc1nc2ccc(-c3ccnc(N)c3)nc2n1-c1ccc(N2CCN(CCC#Cc3ccc(N4CCC(C(=O)N5CCC(n6cc(CNc7cccc8c7C(=O)N(C7CCC(=O)NC7=O)C8=O)cn6)CC5)CC4)nc3)CC2)c(F)c1] |
| 20 | 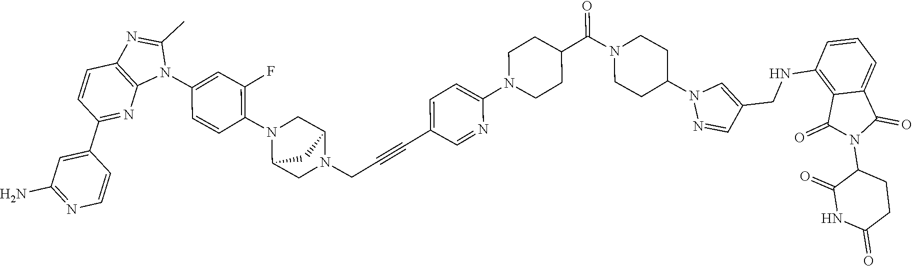 [SMILES: Cc1nc2ccc(-c3ccnc(N)c3)nc2n1-c1ccc(N2C[C@@H]3C[C@H]2CN3CC#Cc2ccc(N3CCC(C(=O)N4CCC(n5cc(CNc6cccc7c6C(=O)N(C6CCC(=O)NC6=O)C7=O)cn5)CC4)CC3)nc2)c(F)c1] |
| 21 | 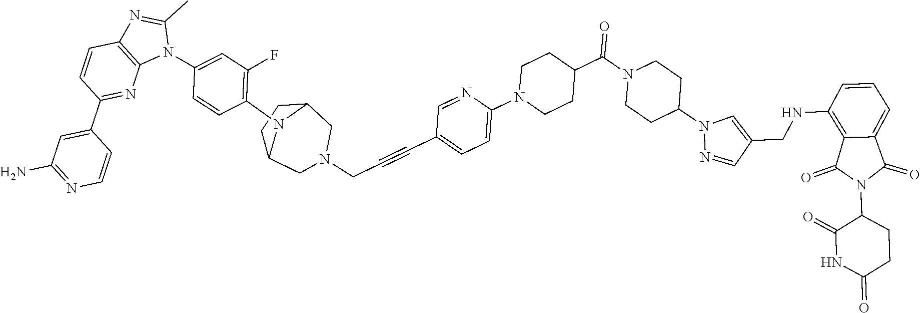 [SMILES: Cc1nc2ccc(-c3ccnc(N)c3)nc2n1-c1ccc(N2C3CCC2CN(CC#Cc2ccc(N4CCC(C(=O)N5CCC(n6cc(CNc7cccc8c7C(=O)N(C7CCC(=O)NC7=O)C8=O)cn6)CC5)CC4)nc2)C3)c(F)c1] |
| 22 | 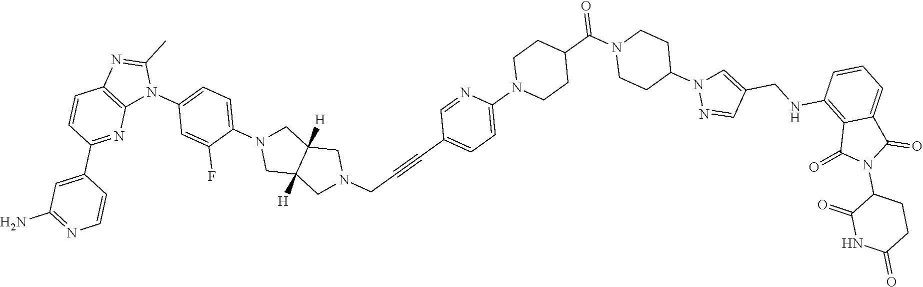 [SMILES: Cc1nc2ccc(-c3ccnc(N)c3)nc2n1-c1ccc(N2C[C@H]3CN(CC#Cc4ccc(N5CCC(C(=O)N6CCC(n7cc(CNc8cccc9c8C(=O)N(C8CCC(=O)NC8=O)C9=O)cn7)CC6)CC5)nc4)C[C@H]3C2)c(F)c1] |
| 23 | 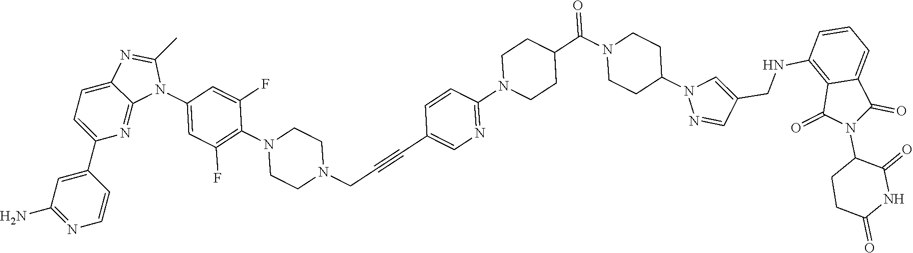 [SMILES: Cc1nc2ccc(-c3ccnc(N)c3)nc2n1-c1cc(F)c(N2CCN(CC#Cc3ccc(N4CCC(C(=O)N5CCC(n6cc(CNc7cccc8c7C(=O)N(C7CCC(=O)NC7=O)C8=O)cn6)CC5)CC4)nc3)CC2)c(F)c1] |
| 24 | 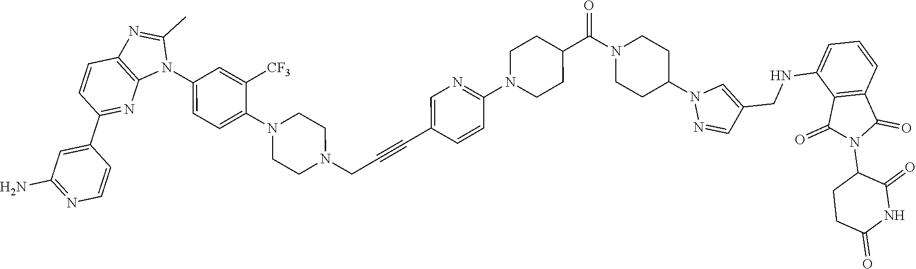 [SMILES: Cc1nc2ccc(-c3ccnc(N)c3)nc2n1-c1ccc(N2CCN(CC#Cc3ccc(N4CCC(C(=O)N5CCC(n6cc(CNc7cccc8c7C(=O)N(C7CCC(=O)NC7=O)C8=O)cn6)CC5)CC4)nc3)CC2)c(C(F)(F)F)c1] |
| 25 | 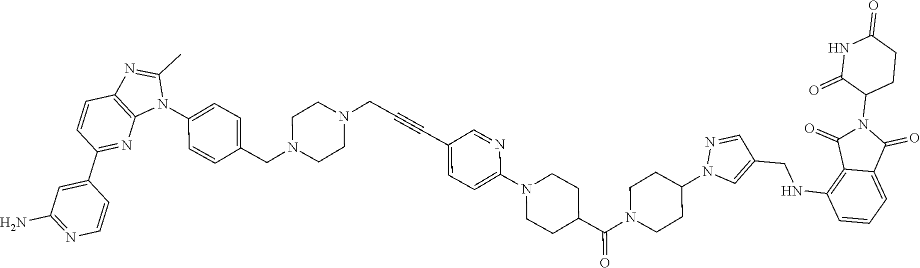 [SMILES: Cc1nc2ccc(-c3ccnc(N)c3)nc2n1-c1ccc(CN2CCN(CC#Cc3ccc(N4CCC(C(=O)N5CCC(n6cc(CNc7cccc8c7C(=O)N(C7CCC(=O)NC7=O)C8=O)cn6)CC5)CC4)nc3)CC2)cc1] |
| 26 | 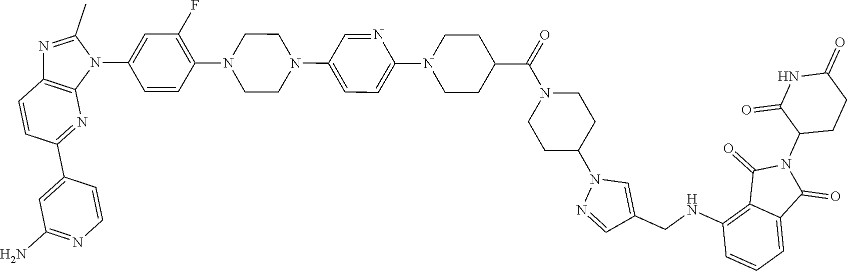 [SMILES: Cc1nc2ccc(-c3ccnc(N)c3)nc2n1-c1ccc(N2CCN(c3ccc(N4CCC(C(=O)N5CCC(n6cc(CNc7cccc8c7C(=O)N(C7CCC(=O)NC7=O)C8=O)cn6)CC5)CC4)nc3)CC2)c(F)c1] |
| 27 | 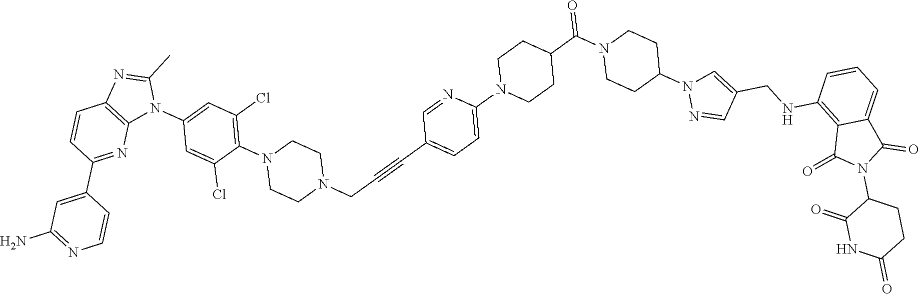 [SMILES: Cc1nc2ccc(-c3ccnc(N)c3)nc2n1-c1cc(Cl)c(N2CCN(CC#Cc3ccc(N4CCC(C(=O)N5CCC(n6cc(CNc7cccc8c7C(=O)N(C7CCC(=O)NC7=O)C8=O)cn6)CC5)CC4)nc3)CC2)c(Cl)c1] |
| 28 | 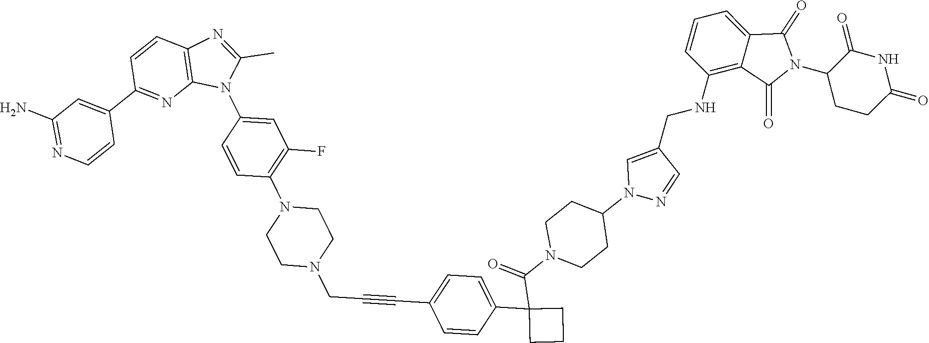 [SMILES: Cc1nc2ccc(-c3ccnc(N)c3)nc2n1-c1ccc(N2CCN(CC#Cc3ccc(C4(C(=O)N5CCC(n6cc(CNc7cccc8c7C(=O)N(C7CCC(=O)NC7=O)C8=O)cn6)CC5)CCC4)cc3)CC2)c(F)c1] |
| 29 | 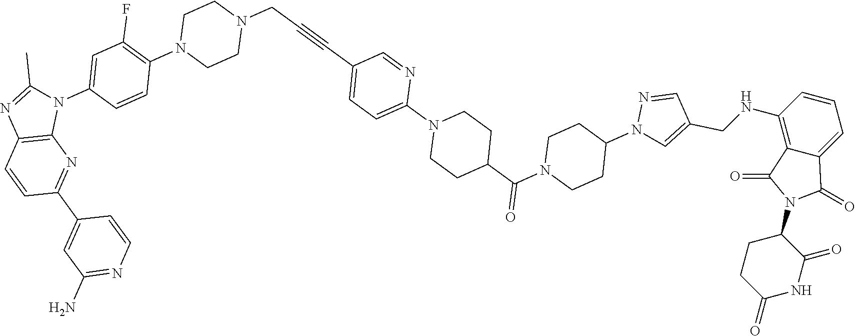 [SMILES: Cc1nc2ccc(-c3ccnc(N)c3)nc2n1-c1ccc(N2CCN(CC#Cc3ccc(N4CCC(C(=O)N5CCC(n6cc(CNc7cccc8c7C(=O)N([C@@H]7CCC(=O)NC7=O)C8=O)cn6)CC5)CC4)nc3)CC2)c(F)c1] |
| 30 | 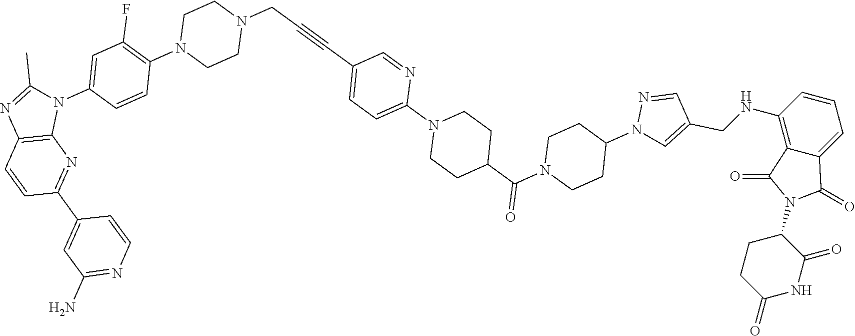 [SMILES: Cc1nc2ccc(-c3ccnc(N)c3)nc2n1-c1ccc(N2CCN(CC#Cc3ccc(N4CCC(C(=O)N5CCC(n6cc(CNc7cccc8c7C(=O)N([C@H]7CCC(=O)NC7=O)C8=O)cn6)CC5)CC4)nc3)CC2)c(F)c1] |
| 31 | 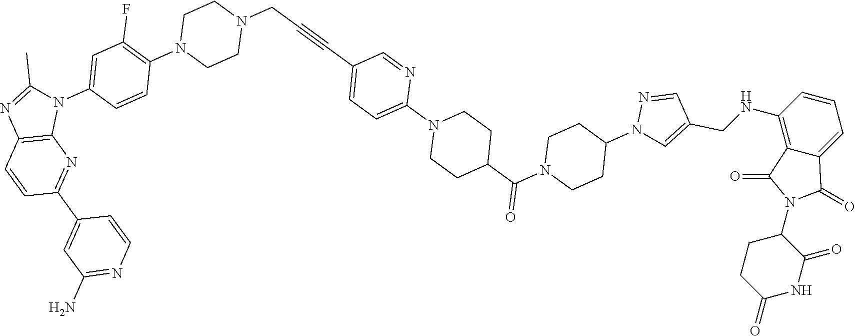 [SMILES: Cc1nc2ccc(-c3ccnc(N)c3)nc2n1-c1ccc(N2CCN(CC#Cc3ccc(N4CCC(C(=O)N5CCC(n6cc(CNc7cccc8c7C(=O)N(C7CCC(=O)NC7=O)C8=O)cn6)CC5)CC4)nc3)CC2)c(F)c1] |
| 32 | 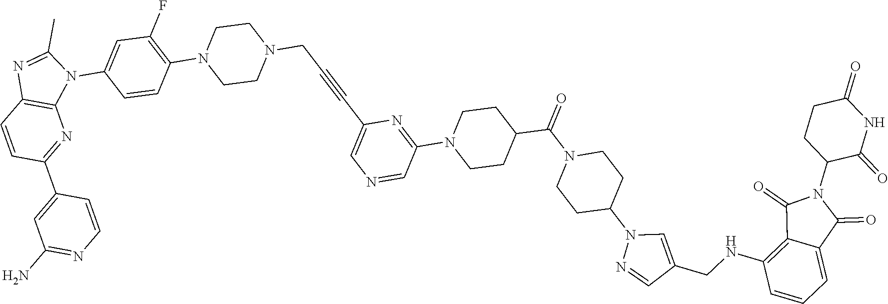 [SMILES: Cc1nc2ccc(-c3ccnc(N)c3)nc2n1-c1ccc(N2CCN(CC#Cc3cncc(N4CCC(C(=O)N5CCC(n6cc(CNc7cccc8c7C(=O)N(C7CCC(=O)NC7=O)C8=O)cn6)CC5)CC4)n3)CC2)c(F)c1] |
| 33 | 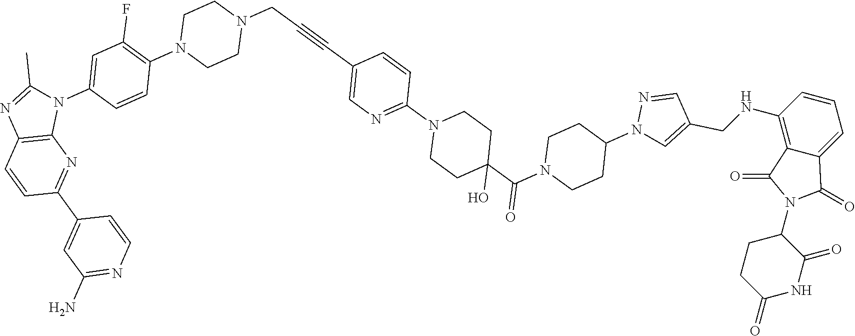 [SMILES: Cc1nc2ccc(-c3ccnc(N)c3)nc2n1-c1ccc(N2CCN(CC#Cc3ccc(N4CCC(O)(C(=O)N5CCC(n6cc(CNc7cccc8c7C(=O)N(C7CCC(=O)NC7=O)C8=O)cn6)CC5)CC4)nc3)CC2)c(F)c1] |
| 34 | 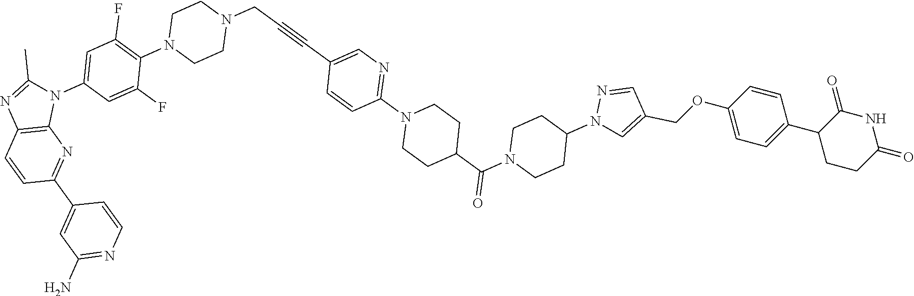 [SMILES: Cc1nc2ccc(-c3ccnc(N)c3)nc2n1-c1cc(F)c(N2CCN(CC#Cc3ccc(N4CCC(C(=O)N5CCC(n6cc(COc7ccc(C8CCC(=O)NC8=O)cc7)cn6)CC5)CC4)nc3)CC2)c(F)c1] |
| 35 | 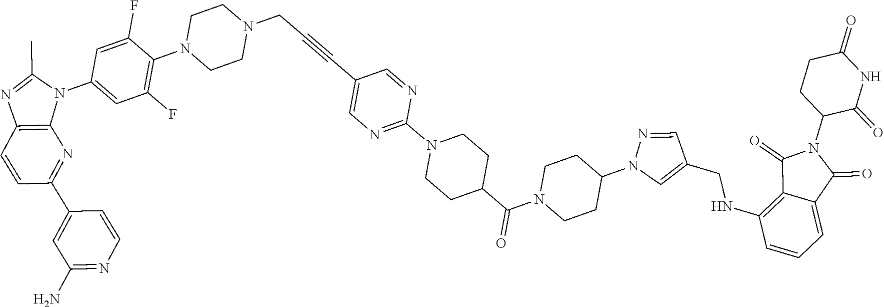 [SMILES: Cc1nc2ccc(-c3ccnc(N)c3)nc2n1-c1cc(F)c(N2CCN(CC#Cc3cnc(N4CCC(C(=O)N5CCC(n6cc(CNc7cccc8c7C(=O)N(C7CCC(=O)NC7=O)C8=O)cn6)CC5)CC4)nc3)CC2)c(F)c1] |
| 36 | 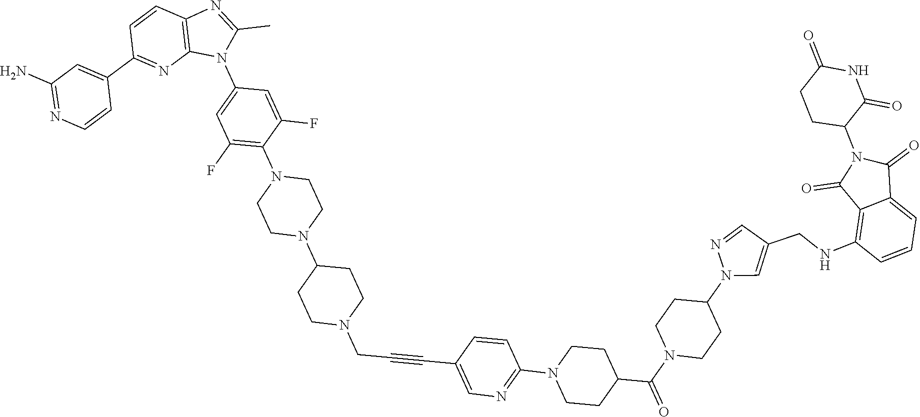 [SMILES: Cc1nc2ccc(-c3ccnc(N)c3)nc2n1-c1cc(F)c(N2CCN(C3CCN(CC#Cc4ccc(N5CCC(C(=O)N6CCC(n7cc(CNc8cccc9c8C(=O)N(C8CCC(=O)NC8=O)C9=O)cn7)CC6)CC5)nc4)CC3)CC2)c(F)c1] |
| 37 | 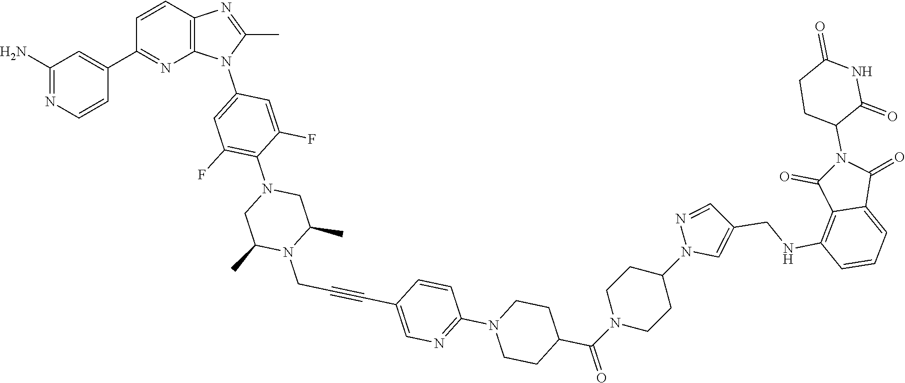 [SMILES: Cc1nc2ccc(-c3ccnc(N)c3)nc2n1-c1cc(F)c(N2C[C@@H](C)N(CC#Cc3ccc(N4CCC(C(=O)N5CCC(n6cc(CNc7cccc8c7C(=O)N(C7CCC(=O)NC7=O)C8=O)cn6)CC5)CC4)nc3)[C@@H](C)C2)c(F)c1] |
| 38 | 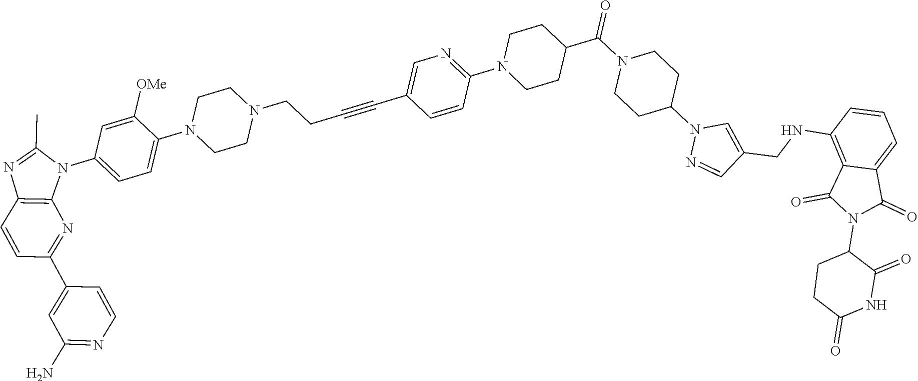 [SMILES: COc1cc(-n2c(C)nc3ccc(-c4ccnc(N)c4)nc32)ccc1N1CCN(CCC#Cc2ccc(N3CCC(C(=O)N4CCC(n5cc(CNc6cccc7c6C(=O)N(C6CCC(=O)NC6=O)C7=O)cn5)CC4)CC3)nc2)CC1] |
| 39 | 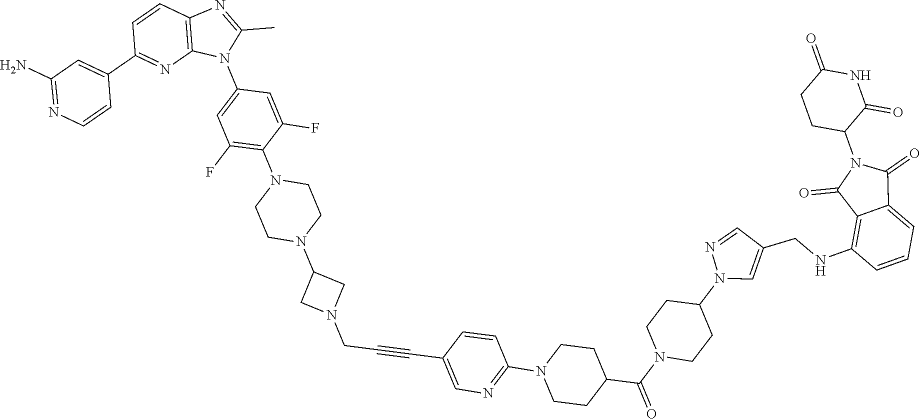 [SMILES: Cc1nc2ccc(-c3ccnc(N)c3)nc2n1-c1cc(F)c(N2CCN(C3CN(CC#Cc4ccc(N5CCC(C(=O)N6CCC(n7cc(CNc8cccc9c8C(=O)N(C8CCC(=O)NC8=O)C9=O)cn7)CC6)CC5)nc4)C3)CC2)c(F)c1] |
| 40 | 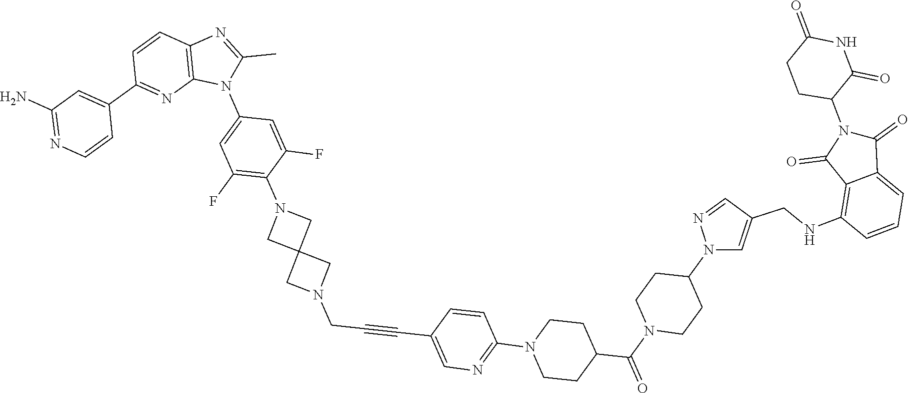 [SMILES: Cc1nc2ccc(-c3ccnc(N)c3)nc2n1-c1cc(F)c(N2CC3(CN(CC#Cc4ccc(N5CCC(C(=O)N6CCC(n7cc(CNc8cccc9c8C(=O)N(C8CCC(=O)NC8=O)C9=O)cn7)CC6)CC5)nc4)C3)C2)c(F)c1] |

### Example 1. Synthesis of (2S,4R)-1-((S)-2-(3-(2-(2-(4-(4-(5-(2-aminopyridin-4-yl)-2-methyl-3H-imidazo[4,5-b]pyridin-3-yl)-2-fluorophenyl)piperazin-1-yl)ethoxy)ethoxy)propanamido)-3,3-dimethylbutanoyl)-4-hydroxy-N-(4-(4-methylthiazol-5-yl)benzyl)pyrrolidine-2-carboxamide (1)

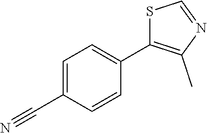

4-(4-methylthiazol-5-yl)benzonitrile (B1)

4-bromobenzonitrile (4.50 g, 24.72 mmol, 1.0 eq.), 4-methylthieazole (4.50 mL, 49.45 mmol, 2.0 eq.), potassium acetate (4.85 g, 49.45 mmol, 2.0 eq.), and Pd(OAc) 2 (55 mg, 0.24 mmol, 0.01 eq.) were dissolved in dimethylacetamide (74 mL) and stirred under argon for 10 minutes. The reaction mixture was heated at 150° C. for 24 hours. The reaction mixture was diluted with ethyl acetated, and washed with water (4×). The organic layer was dried over sodium sulfate, filtered, and concentrated under reduced pressure. The crude residue was purified by flash column chromatography to give 4-(4-methylthiazol-5-yl)benzonitrile (B1) (4.70 g, 23.48 mmol, 95% yield, eluting at 1% MeOH in DCM), a brown solid. ¹H NMR (500 MHz, CDCl₃) δ 8.75 (s, 1H), 7.72 (d, J=8.3 Hz, 2H), 7.56 (d, J=8.5 Hz, 2H), 2.56 (s, 3H). ¹³C NMR (126 MHz, CDCl₃) δ 151.70, 150.20, 137.02, 132.64, 129.86, 118.58, 111.67, 77.41, 77.16, 76.91, 16.47.

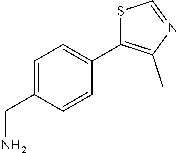

### (4-(4-methylthiazol-5-yl)phenyl)methanamine (B2)

4-(4-methylthiazol-5-yl)benzonitrile (B1) (4.58 g, 22.87 mmol, 1.0 eq.) was dissolved in MeOH (220 mL) and cooled to 4 C. Cobalt (II) chloride hexahydrate (8.16 g, 34.31 mmol, 1.50 eq.) was added, followed by slow addition of sodium borohydride (4.32 g, 114.4 mmol, 5.0 eq.). After 90 minutes, the reaction was quenched by the addition of water and ammonium hydroxide. The mixture was filtered through celite and washed with DCM. The filtrate was concentrated under reduced pressure, extracted with ethyl acetate (3×40 mL) and washed with water. The organic layer was dried over sodium sulfate and concentrated under reduced pressure. The crude residue was purified by flash column chromatography to give (4-(4-methylthiazol-5-yl)phenyl)methanamine (B2) (1.33 g, 6.52 mmol, 28%, eluting at 8% MeOH in DCM), an orange oil. ¹H NMR (500 MHz, CDCl₃) δ 8.50 (s, 1H), 7.29-7.19 (m, 4H), 3.75 (s, 2H), 2.37 (s, 3H), 1.35 (s, 2H). ¹³C NMR (126 MHz, CDCl₃) δ 150.26, 148.54, 143.23, 131.86, 130.53, 129.56, 127.56, 77.41, 77.16, 76.91, 46.24, 16.20.

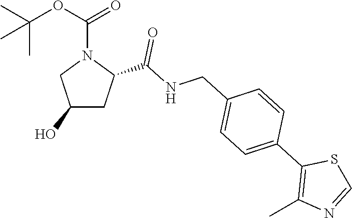 [SMILES: Cc1ncsc1-c1ccc(CNC(=O)[C@@H]2C[C@@H](O)CN2C(=O)OC(C)(C)C)cc1]

### tert-butyl(2S,4R)-4-hydroxy-2-((4-(4-methylthiazol-5-yl)benzyl)carbamoyl)pyrrolidine-1-carboxylate (B3)

1-(tert-butoxycarbonyl)-4-hydroypyrrolidine-2-carboxylic acid (B2) (1.25 g, 5.41 mmol, 1.0 eq.) was dissolved in DMF (52 mL) when EDC, HCl (1.60 g, 7.03 mmol, 1.3 eq.) and HOBt (1.55 g, 8.12 mmol, 1.5 eq., 80% Wt.). After 5 minutes of stirring, (4-(4-methylthiazol-5-yl)phenyl)methanamine (1.10 g, 5.41 mmol, 1.0 eq.) was added. After 24 hours, the reaction was diluted with 50 mL ethyl acetate, washed with brine (2×) and saturated sodium bicarbonate (2 x). The organic layer was dried over sodium sulfate, concentrated under reduced pressure followed by serial concentrations and dilutions with acetonitrile. The crude residue was purified by flash column chromatography to give tert-butyl(2S,4R)-4-hydroxy-2-((4-(4-methylthiazol-5-yl)benzyl)carbamoyl)pyrrolidine-1-carboxylate (B3) (2.02 g, 4.84 mmol, 89% yield), a yellow oil.

¹H NMR (500 MHz, CDCl₃) δ 8.70 (s, 1H), 7.37 (d, J=7.8 Hz, 2H), 7.32 (d, J=7.8 Hz, 2H), 5.29 (s, 1H), 4.47 (s, 5H), 3.54-3.45 (m, 2H), 2.79 (s, 1H), 2.51 (s, 3H), 2.24-1.99 (m, 1H), 1.43 (s, 9H). LCMS (ESI) C₂₁H₂₇N₃O₄S requires 417.17, found 418 (M/Z+H).

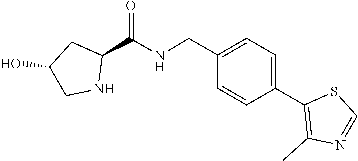 [SMILES: Cc1ncsc1-c1ccc(CNC(=O)[C@@H]2C[C@@H](O)CN2)cc1]

### (2S,4R)-4-hydroxy-N-(4-(4-methylthiazol-5-yl)benzyl)pyrrolidine-2-carboxamide, Trifluoroacetic acid (B4)

TFA (25.00 mL, 320 mmol, 67 eq.) and DCM (25 mL) were added to a sealed 5 ml MWV equipped with a magnetic stir bar containing tert-butyl(2S,4R)-4-hydroxy-2-((4-(4-methylthiazol-5-yl)benzyl)carbamoyl)pyrrolidine-1-carboxylate (2.02 g, 4.84 mmol, 1.0 eq.). After 4 hours the reaction was determined complete by LCMS and the reaction mixture was concentrated under reduced pressure to give (2S,4R)-4-hydroxy-N-(4-(4-methylthiazol-5-yl)benzyl)pyrrolidine-2-carboxamide, trifluoroacetic acid (B4) in quantitative yield and was used without further purification.

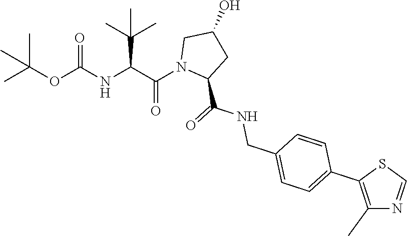 [SMILES: Cc1ncsc1-c1ccc(CNC(=O)[C@@H]2C[C@@H](O)CN2C(=O)[C@@H](NC(=O)OC(C)(C)C)C(C)(C)C)cc1]

### tert-butyl ((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)carbamate (B5)

To a solution of preparative compound (2S,4R)-4-hydroxy-N-(4-(4-methylthiazol-5-yl)benzyl)pyrrolidine-2-carboxamide, trifluoroacetic acid (B4) (2.09 g, 4.84 mmol, 1.0 eq.) in DMF (43 mL) was added(S)-2-((tert-butoxycarbonyl)amino)-3,3-dimethylbutanoic acid (1.12 g, 4.84 mmol, 1.0 eq.) and the solution was stirred at room temperature. N-ethyl-N-isopropylpropan-2-amine (3.38 mL, 19.39 mmol, 4.0 eq.) was added dropwise, and the mixture was stirred for 5 minutes at room temperature. HATU (2.02 g, 5.33 mmol, 1.1 eq.) was added, and the mixture was stirred at room temperature for another 30 min. Water was added, and the mixture was extracted with ethyl acetate (3×). The combined organic phases were washed with brine (x2), dried over Na₂SO₄, and evaporated under reduced pressure to give the corresponding crude. The resulting crude oil was purified by flash column chromatography to give tert-butyl ((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)carbamate (B5) (2.5331 g, 3.6 mmol, 74%, 75% Wt.). LCMS (ESI) C₂₇H₃₈N₄O₅S requires 530.25, found 531 (M/Z+H).

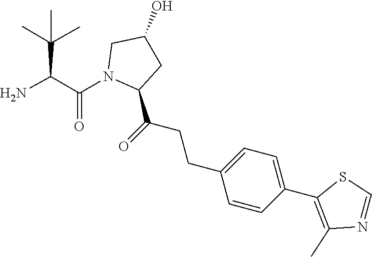 [SMILES: Cc1ncsc1-c1ccc(CCC(=O)[C@@H]2C[C@@H](O)CN2C(=O)[C@@H](N)C(C)(C)C)cc1]

### (2S,4R)-1-((S)-2-amino-3,3-dimethylbutanoyl)-4-hydroxy-N-(4-(4-methylthiazol-5-yl)benzyl)pyrrolidine-2-carboxamide, HCl (B6)

To a 100 mL RBF containing tert-butyl ((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)carbamate (2.5331 g, 75% Wt, 1 Eq, 3.5800 mmol) in DCM (24 mL) was added HCl (36 g, 24 mL, 4 molar, 27 Eq, 96 mmol). The reaction mixture was stirred overnight where it was then concentrated under reduced pressure to give (2S,4R)-1-((S)-2-amino-3,3-dimethylbutanoyl)-4-hydroxy-N-(4-(4-methylthiazol-5-yl)benzyl)pyrrolidine-2-carboxamide, HCl (B6) (1.2047 g, 2.5795 mmol, 72.054%), a yellow solid. LCMS (ESI) C₂₂H₃₀N₄O₃S requires 430.20, found 431 (M/Z+H).

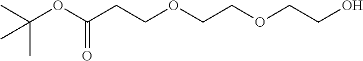 [SMILES: CC(C)(C)OC(=O)CCOCCOCCO]

### tert-butyl 3-(2-(2-hydroxyethoxy)ethoxy)propanoate (1a)

To suspension of sodium hydride (34 mg, 8.58 mmol, 1.1 eq., 60% Wt.) in 1,4-Dioxane (15.6 mL) was added diethylene glycol (2.611 mL, 27.30 mmol, 3.5 eq.) at 0° C. After 15 min at 0° C., tert-butyl acrylate (1.14 mL, 7.80 mmol, 1.0 eq.) was added and the mixture was stirred 16 h at room temperature. A solution of 1M HCl was added dropwise until pH ˜3 was reached, and a saturated solution of sodium bicarbonate was slowly added. The resulting mixture was extracted with DCM and the combined organic extracts were dried over sodium sulfate and concentrated under reduced pressure. The crude residue was purified by flash column chromatography to give tert-butyl 3-(2-(2-hydroxyethoxy)ethoxy)propanoate (la) (1.269 g, 5.416 mmol, 69.42%), a clear liquid. ¹H NMR (400 MHz, CDCl₃) δ 3.76-3.68 (m, 4H), 3.68-3.55 (m, 6H), 2.50 (td, J=6.4, 1.0 Hz, 2H), 2.46 (bs, 1H), 1.44 (s, 9H). ¹³C NMR (101 MHz, CDCl₃) δ 171.08, 80.82, 77.48, 77.16, 76.84, 72.63, 70.50, 70.48, 66.98, 61.92, 36.32, 28.21. Note: The NaH was 60% by weight in mineral oil and was washed with hexanes before use.

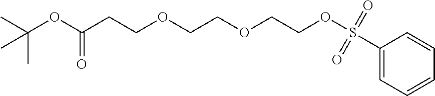 [SMILES: CC(C)(C)OC(=O)CCOCCOCCOS(=O)(=O)c1ccccc1]

### tert-butyl 3-(2-(2-((phenylsulfonyl)oxy)ethoxy)ethoxy)propanoate (1b)

To a solution of tert-butyl 3-(2-(2-hydroxyethoxy)ethoxy)propanoate (la) (1.00 g, 4.26 mmol, 1.0 eq.) in DCM (8.50 mL) were added benzenesulfonyl chloride (0.76 mL, 5.97 mmol, 1.4 Eq), triethylamine (0.65 mL, 4.69 mmol, 1.1 eq.) and DMAP (52 mg, 0.42 mmol, 0.1 eq.). The mixture was stirred 16 hours at room temperature and a saturated solution of ammonium chloride was then added. The mixture was extracted with DCM and the combined organic extracts were dried over sodium sulfate and concentrated under reduced pressure. The crude residue was purified by flash column chromatography to afford tert-butyl 3-(2-(2 ((phenylsulfonyl)oxy)ethoxy)ethoxy)propanoate (1b) (1.33 g, 3.56 mmol, 83%), as a clear liquid.

¹H NMR (400 MHz, CDCl₃) δ 7.96-7.89 (m, 2H), 7.71-7.61 (m, 1H), 7.61-7.52 (m, 2H), 4.23-4.16 (m, 2H), 3.76-3.64 (m, 4H), 3.63-3.50 (m, 4H), 2.49 (t, J=6.5 Hz, 2H), 1.45 (s, 9H). ¹³C NMR (101 MHz, CDCl₃) δ 170.93, 136.18, 133.85, 129.31, 128.02, 80.64, 70.72, 70.40, 69.56, 68.75, 66.99, 36.32, 28.18.

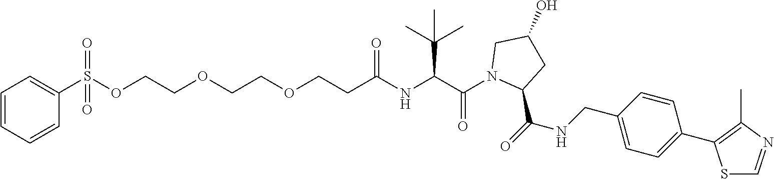 [SMILES: Cc1ncsc1-c1ccc(CNC(=O)[C@@H]2C[C@@H](O)CN2C(=O)[C@@H](NC(=O)CCOCCOCCOS(=O)(=O)c2ccccc2)C(C)(C)C)cc1]

### 2-(2-(3-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-3-oxopropoxy)ethoxy)ethyl benzenesulfonate (1d)

tert-butyl 3-(2-(2-((phenylsulfonyl)oxy)ethoxy)ethoxy)propanoate (1b) (500 mg, 1.34 mmol, 1.0 eq.) was dissolved in DCM (4.1 mL) and TFA (0.51 mL, 6.68 mmol, 5.0 eq.) was added. The reaction mixture was stirred for 2 hours at room temperature where it was then concentrated under reduced pressure to give (1c) 3-(2-(2-((phenylsulfonyl)oxy)ethoxy)ethoxy)propanoic acid, a clear oil, and was used without further purification. LCMS (ESI) C₁₃H₁₈₀₇S requires 318.34, found 319.65 (M/Z+H). HATU (58 mg, 1.1 Eq, 0.15 mmol) was added to a 5 ml MWV containing 3-(2-(2-((phenylsulfonyl)oxy)ethoxy)ethoxy)propanoic acid (1c) (44 mg, 0.14 mmol, 1.0 eq.), (2S,4R)-1-((S)-2-amino-3,3-dimethylbutanoyl)-4-hydroxy-N-(4-(4-methylthiazol-5-yl)benzyl)pyrrolidine-2-carboxamide (B6) (60 mg, 1 Eq, 0.14 mmol), DIPEA (0.15 mL, 0.84 mmol, 6.0 eq.) and DMF (1.4 mL). The reaction mixture was stirred at room temperature for 1 hour and the reaction was quenched with brine (15 ml) and extracted with DCM (3×15 ml). The organic layer was dried over sodium sulfate, concentrated under reduced pressure, and purified via flash column chromatography to give 2-(2-(3-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-3-oxopropoxy)ethoxy)ethyl benzenesulfonate (1d) (92 mg, 0.13 mmol, 90% yield, eluting at 8% MeOH in DCM), a white solid. ¹H NMR (500 MHz, CDCl₃) δ 8.69 (s, 1H), 7.91-7.85 (m, 2H), 7.67-7.60 (m, 1H), 7.53 (t, J=7.8 Hz, 2H), 7.40 (t, J=6.0 Hz, 1H), 7.33 (s, 4H), 6.99 (d, J=8.3 Hz, 1H), 4.69 (t, J=8.0 Hz, 1H), 4.58-4.44 (m, 3H), 4.33 (dd, J=15.0, 5.4 Hz, 1H), 4.22-4.13 (m, 2H), 4.06 (d, J=11.4 Hz, 1H), 3.73-3.63 (m, 5H), 3.63-3.53 (m, 5H), 3.16 (qd, J=7.5, 4.4 Hz, 1H), 2.49 (s, 3H), 2.47-2.35 (m, 2H), 2.20-2.08 (m, 1H), 0.93 (s, 9H). LCMS (ESI) C₃₅H₄₆N₄O₉S₂ requires 730.89, found 731.14 (M/Z+H).

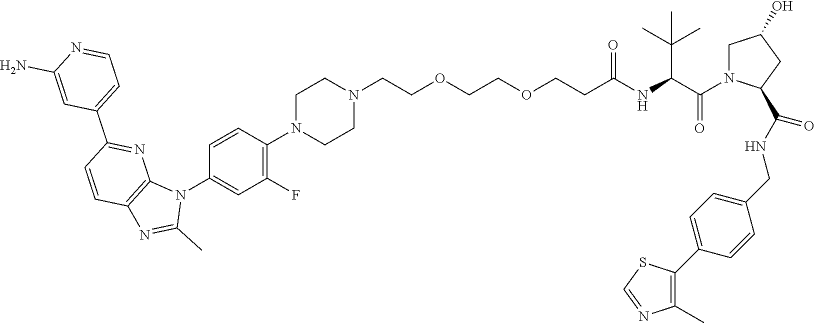 [SMILES: Cc1ncsc1-c1ccc(CNC(=O)[C@@H]2C[C@@H](O)CN2C(=O)[C@@H](NC(=O)CCOCCOCCN2CCN(c3ccc(-n4c(C)nc5ccc(-c6ccnc(N)c6)nc54)cc3F)CC2)C(C)(C)C)cc1]

### (2S,4R)-1-((S)-2-(3-(2-(2-(4-(4-(5-(2-aminopyridin-4-yl)-2-methyl-3H-imidazo[4,5-b]pyridin-3-yl)-2-fluorophenyl)piperazin-1-yl)ethoxy)ethoxy)propanamido)-3,3-dimethylbutanoyl)-4-hydroxy-N-(4-(4-methylthiazol-5-yl)benzyl)pyrrolidine-2-carboxamide (1)

To a 5 mL MWV equipped with a magnetic stir bar containing 4-(3-(3-fluoro-4-(piperazin-1-yl)phenyl)-2-methyl-3H-imidazo[4,5-b]pyridin-5-yl)pyridin-2-amine (A7) (16 mg, 40 μmol, 1.0 eq.), 2-(2-(3-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-3-oxopropoxy)ethoxy)ethyl benzenesulfonate (1d) (32 mg, 44 μmol, 1.1 eq.), potassium carbonate (11 mg, 79 μmol, 2.0 eq.), and sodium iodide (3.0 mg, 20 μmol, 1.1 eq.) was added DMF (0.50 mL). The reaction mixture was stirred at 80° C. for 16 hours where it was diluted and extracted with DCM (3×10 mL) and washed with water. The aqueous was extracted with ethyl acetate (5×10 mL) and washed with saturated sodium bicarbonate. The organic layers were recombined, dried over sodium sulfate, and concentrated under reduced pressure. The residue was purified by flash column chromatography to give (2S,4R)-1-((S)-2-(3-(2-(2-(4-(4-(5-(2-aminopyridin-4-yl)-2-methyl-3H-imidazo[4,5-b]pyridin-3-yl)-2-fluorophenyl)piperazin-1-yl)ethoxy)ethoxy)propanamido)-3,3-dimethylbutanoyl)-4-hydroxy-N-(4-(4-methylthiazol-5-yl)benzyl)pyrrolidine-2-carboxamide (1) (11 mg, 11.8 μmol, 30% yield), a yellow solid. ¹H NMR (500 MHz, MeOD) δ 8.83 (s, 1H), 8.31 (bs, 2H), 8.14-8.06 (m, 1H), 7.99-7.92 (m, 1H), 7.87 (d, J=6.1 Hz, 1H), 7.47-7.27 (m, 10H), 4.67 (s, 1H), 4.62-4.46 (m, 3H), 4.35 (d, J=15.4 Hz, 1H), 3.92-3.72 (m, 6H), 3.72-3.60 (m, 5H), 3.45 (s, 4H), 3.31 (p, J=1.7 Hz, 6H), 3.24 (t, J=4.5 Hz., 2H), 2.56 (d, J=2.8 Hz, 4H), 2.43 (s, 3H), 2.27-2.17 (m, 1H), 2.09 (tdd, J=13.3, 10.0, 4.6 Hz, 1H), 1.04 (s, 9H). LCMS (ESI) C₅₁H₆₂FN₁₁O₆S requires 975.45, found 976 (M/Z+H).

### Example 2. Synthesis of (2S,4R)-1-((S)-1-(4-(4-(5-(6-aminopyrimidin-4-yl)-2-methyl-3H-imidazo[4,5-b]pyridin-3-yl)-2-fluorophenyl)piperazin-1-yl)-14-(tert-butyl)-12-oxo-3,6,9-trioxa-13-azapentadecan-15-oyl)-4-hydroxy-N-(4-(4-methylthiazol-5-yl)benzyl)pyrrolidine-2-carboxamide (2)

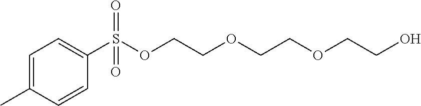 [SMILES: Cc1ccc(S(=O)(=O)OCCOCCOCCO)cc1]

### 2-(2-(2-hydroxyethoxy)ethoxy)ethyl 4-methylbenzenesulfonate (2a)

4-Toluenesulfonyl chloride (900 mg, 4.72 mmol, 1.0 eq.), silver oxide (1.53 g, 6.61 mmol, 1.4 eq.), and potassium iodide (212 mg, 1.27 mmol, 0.27 eq.) were added to a 250 mL RBF containing 2,2′-(ethane-1,2-diylbis(oxy))bis(ethan-1-ol) (1.21 g, 1.08 mL, 8.03 mmol, 1.7 eq.) in DCM (47 mL) at 0° C. The resulting suspension was protected from light and stirred at room temperature for 1 h. The reaction mixture was filtered through celite and the celite pad was washed with DCM. The filtrate was extracted with DCM (4×15 ml) and washed with water (10 ml). The organic layer was dried over sodium sulfate, filtered over, concentrated under reduced pressure and purified by flash column chromatography to give 2-(2-(2-hydroxyethoxy)ethoxy)ethyl 4-methylbenzenesulfonate (2a) (1.11 g, 3.67 mmol, 77%), a light-yellow oil. ¹H NMR (500 MHz, CDCl₃) δ 7.79 (d, J=8.3 Hz, 2H), 7.33 (d, J=8.1 Hz, 2H), 4.18-4.13 (m, 2H), 3.69 (q, J=4.7 Hz, 4H), 3.59 (s, 3H), 3.57-3.52 (m, 2H), 2.43 (s, 3H), 2.27 (s, 1H).

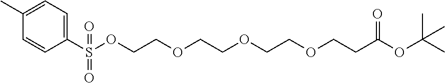 [SMILES: Cc1ccc(S(=O)(=O)OCCOCCOCCOCCC(=O)OC(C)(C)C)cc1]

### tert-butyl 3-(2-(2-(2-(tosyloxy)ethoxy)ethoxy)ethoxy)propanoate (2b)

To a suspension of NaH (159 mg, 3.97 mmol, 1.1 eq., 60% Wt.) in 1,4-dioxane (8.50 mL) was added 2-(2-(2-hydroxyethoxy)ethoxy)ethyl 4-methylbenzenesulfonate (2a) (1.10 g, 3.61 mmol, 1.0 eq.) at 0° C. After 15 minutes at 0° C., tert-butyl acrylate (0.53 mL, 3.61 mmol, 1.0 eq.) was added and the reaction was stirred for 20 hours at room temperature. The reaction mixture was quenched with a saturated solution of ammonium chloride, extracted with DCM (3×20 mL), and washed with water. The organic layers were recombined, dried over sodium sulfate, and concentrated under reduced pressure. The residue was purified via flash column chromatography to give tert-butyl 3-(2-(2-(2-(tosyloxy)ethoxy)ethoxy)ethoxy)propanoate (2b) (363 mg, 0.83 mmol, 23% yield), a clear oil. ¹H NMR (500 MHz, CDCl₃) δ 7.82-7.76 (m, 2H), 7.37-7.30 (m, 2H), 4.18-4.12 (m, 2H), 3.72-3.65 (m, 4H), 3.58 (d, J=6.9 Hz, 8H), 2.49 (t, J=6.6 Hz, 2H), 2.44 (s, 3H), 1.44 (s, 9H). ¹³C NMR (126 MHz, CDCl₃) δ 171.03, 144.91, 133.16, 129.95, 128.12, 80.65, 77.42, 77.16, 76.91, 70.89, 70.67, 70.48, 69.37, 68.82, 67.03, 66.99, 36.39, 28.23, 21.77.

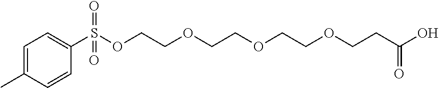 [SMILES: Cc1ccc(S(=O)(=O)OCCOCCOCCOCCC(=O)O)cc1]

### 3-(2-(2-(2-(tosyloxy)ethoxy)ethoxy)ethoxy)propanoic acid (2c)

Trifluoroacetic acid (0.32 mL, 4.21 mmol, 5.0 eq.) was added to a 5 mL MWV equipped with a magnetic stir bar containing tert-butyl 3-(2-(2-(2-(tosyloxy)ethoxy)ethoxy)ethoxy)propanoate (2b) (364 mg, 842 μmol, 1.0 eq.) in DCM (3.0 mL). The reaction mixture was stirred for 2 hours at room temperature where it was then diluted with DCM and concentrated under reduced pressure to give 3-(2-(2-(2-(tosyloxy)ethoxy)ethoxy)ethoxy)propanoic acid (2c) (317 mg, 842 μmol, 100%), a clear oil. ¹H NMR (500 MHz, CDCl₃) δ 7.80 (d, J=8.4 Hz, 2H), 7.34 (d, J=7.9 Hz, 2H), 4.19-4.13 (m, 2H), 3.76 (t, J=6.2 Hz, 2H), 3.71-3.66 (m, 2H), 3.65-3.60 (m, 3H), 3.59 (s, 4H), 2.63 (t, J=6.1 Hz, 2H), 2.44 (s, 3H), 1.44 (s, 1H). ¹³C NMR (126 MHz, CDCl₃) δ 175.23, 144.97, 133.10, 129.97, 128.13, 77.41, 77.16, 76.90, 70.88, 70.66, 70.54, 70.46, 69.42, 68.84, 66.43, 34.86, 28.23, 21.78.

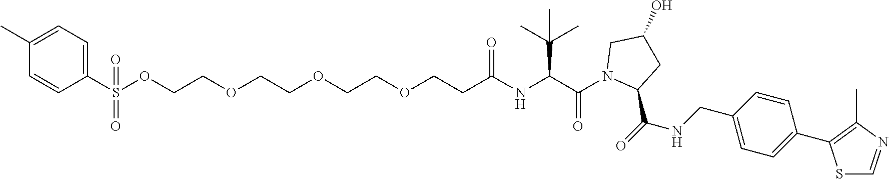 [SMILES: Cc1ccc(S(=O)(=O)OCCOCCOCCOCCC(=O)N[C@H](C(=O)N2C[C@H](O)C[C@H]2C(=O)NCc2ccc(-c3scnc3C)cc2)C(C)(C)C)cc1]

(S)-14-((2S,4R)-4-hydroxy-2-((4-(4-methylthiazol-5-yl)benzyl)carbamoyl)pyrrolidine-1-carbonyl)-15,15-dimethyl-12-oxo-3,6,9-trioxa-13-azahexadecyl 4-methylbenzenesulfonate (2d) HATU (78 mg, 0.20 mmol, 1.1 eq.) and DIPEA (0.19 mL, 1.1 mmol, 6.0 eq.) was added to a 5 mL MWV containing 3-(2-(2-(2-(tosyloxy)ethoxy)ethoxy)ethoxy)propanoic acid (2c) (70 mg, 0.19 mmol, 1.0 eq.), (2S,4R)-1-((S)-2-amino-3,3-dimethylbutanoyl)-4-hydroxy-N-(4-(4-methylthiazol-5-yl)benzyl)pyrrolidine-2-carboxamide (B6) (80 mg, 0.19 mmol, 1.0 eq.), and DMF (1.60 mL). The reaction mixture was stirred overnight at room temperature and the reaction was quenched with brine (15 mL) and extracted with DCM (5×15 ml). The organic layer was dried over sodium sulfate, concentrated under reduced pressure, and purified via flash column chromatography to give(S)-14-((2S,4R)-4-hydroxy-2-((4-(4-methylthiazol-5-yl)benzyl)carbamoyl)pyrrolidine-1-carbonyl)-15,15-dimethyl-12-oxo-3,6,9-trioxa-13-azahexadecyl 4-methylbenzenesulfonate (2d) (66 mg, 75 μmol, 45% yield), a clear semisolid. ¹H NMR (500 MHz, CDCl₃) δ 8.66 (s, 1H), 7.76 (d, J=8.2 Hz, 2H), 7.41 (t, J=6.0 Hz, 1H), 7.36-7.30 (m, 6H), 6.98 (d, J=8.3 Hz, 1H), 4.69 (t, J=8.0 Hz, 1H), 4.56-4.42 (m, 3H), 4.33 (dd, J=15.1, 5.4 Hz, 1H), 4.17-4.09 (m, 2H), 4.06 (d, J=11.4 Hz, 1H), 3.72-3.63 (m, 4H), 3.63-3.53 (m, 8H), 2.49 (s, 3H), 2.47-2.42 (m, 2H), 2.42 (s, 3H), 2.17-2.07 (m, 1H), 1.49-1.37 (m, 2H), 0.93 (s, 9H). LCMS (ESI) C₃₈H₅₂N₄O₁₀S₂ requires 788.31, found 789.12 (M/Z+H).

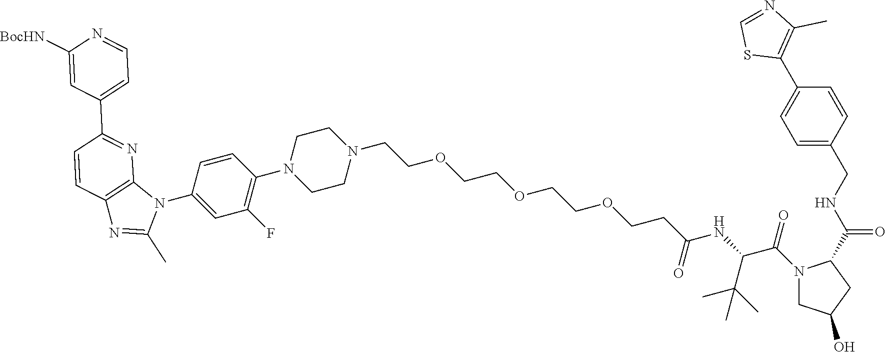 [SMILES: Cc1ncsc1-c1ccc(CNC(=O)[C@@H]2C[C@@H](O)CN2C(=O)[C@@H](NC(=O)CCOCCOCCOCCN2CCN(c3ccc(-n4c(C)nc5ccc(-c6ccnc(NC(=O)OC(C)(C)C)c6)nc54)cc3F)CC2)C(C)(C)C)cc1]

### tert-butyl (4-(3-(3-fluoro-4-(4-((S)-14-((2S,4R)-4-hydroxy-2-((4-(4-methylthiazol-5-yl)benzyl)carbamoyl)pyrrolidine-1-carbonyl)-15,15-dimethyl-12-oxo-3,6,9-trioxa-13-azahexadecyl)piperazin-1-yl)phenyl)-2-methyl-3H-imidazo[4,5-b]pyridin-5-yl)pyridin-2-yl)carbamate (2c)

To a 5 mL MWV equipped with a magnetic stir bar containing tert-butyl (4-(3-(3-fluoro-4-(piperazin-1-yl)phenyl)-2-methyl-3H-imidazo[4,5-b]pyridin-5-yl)pyridin-2-yl)carbamate (40 mg, 79 μmol, 1.0 eq.), (S)-14-((2S,4R)-4-hydroxy-2-((4-(4-methylthiazol-5-yl)benzyl)carbamoyl)pyrrolidine-1-carbonyl)-15,15-dimethyl-12-oxo-3,6,9-trioxa-13-azahexadecyl 4-methylbenzenesulfonate (2d) (69 mg, 87 μmol, 1.1 eq.) sodium iodide (6.0 mg, 40 μmol, 0.5 eq.), and potassium carbonate (22 mg, 160 μmol, 2.0 eq.) was added DMF (0.5 mL). The reaction mixture was stirred at 80° C. for 10 hours where it was then extracted with DCM (5×10 mL) and washed with water. The organic layers were recombined, dried over sodium sulfate, and concentrated under reduced pressure. The residue was purified by flash column chromatography to give tert-butyl (4-(3-(3-fluoro-4-(4-((S)-14-((2S,4R)-4-hydroxy-2-((4-(4-methylthiazol-5-yl)benzyl)carbamoyl)pyrrolidine-1-carbonyl)-15,15-dimethyl-12-oxo-3,6,9-trioxa-13-azahexadecyl)piperazin-1-yl)phenyl)-2-methyl-3H-imidazo[4,5-b]pyridin-5-yl)pyridin-2-yl)carbamate (2e) (47 mg, 42 μmol, 53% yield), a yellow solid. LCMS (ESI) C₅₈H₇₄FN₁₁O₉S requires 1119.53, found 561.35 (M/2+1). ¹H NMR (500 MHz, MeOD) δ 8.84 (s, 1H), 8.47-8.43 (m, 1H), 8.22 (d, J=5.3 Hz, 1H), 8.05 (d, J=8.4 Hz, 1H), 7.91 (d, J=8.4 Hz, 1H), 7.60 (dd, J=5.4, 1.6 Hz, 1H), 7.48-7.26 (m, 7H), 7.22 (t, J=8.9 Hz, 1H), 4.64 (d, J=4.2 Hz, 1H), 4.61-4.53 (m, 2H), 4.52-4.46 (m, 2H), 4.39-4.30 (m, 1H), 3.92-3.83 (m, 1H), 3.81-3.53 (m, 13H), 3.31 (p, J=1.7 Hz, 4H), 3.26-3.21 (m, 5H), 2.79-2.74 (m, 3H), 2.69 (t, J=5.6 Hz, 2H), 2.55 (s, 4H), 2.44 (s, 3H), 2.24-2.17 (m, 1H), 2.11-2.04 (m, 1H), 1.53 (s, 9H), 1.03 (s, 9H). ¹³C NMR (126 MHz, MeOD) δ 173.05, 172.31, 170.72, 156.09, 155.40, 154.13, 153.10, 153.04 151.41, 148.86, 148.62, 147.85, 147.61, 138.86, 134.21, 131.99, 130.08, 128.93, 127.57, 126.29, 123.74, 119.11, 119.08, 116.44, 115.87, 115.53, 115.35, 109.90, 80.35, 70.20, 70.14, 70.05, 70.03, 69.68, 68.17, 66.93, 59.42, 57.53, 57.35, 56.60, 53.21, 49.68, 48.46, 42.30, 37.55, 35.98, 35.41, 27.27, 25.68, 14.48, 13.58. Note: ¹³C splitting from C-F was not measured due to molecule complexity, all peaks were identified instead. LCMS (ESI) C₅₈H₇₄FN₁₁O₉S requires 1119.48, found 1020 (M/Z+H).

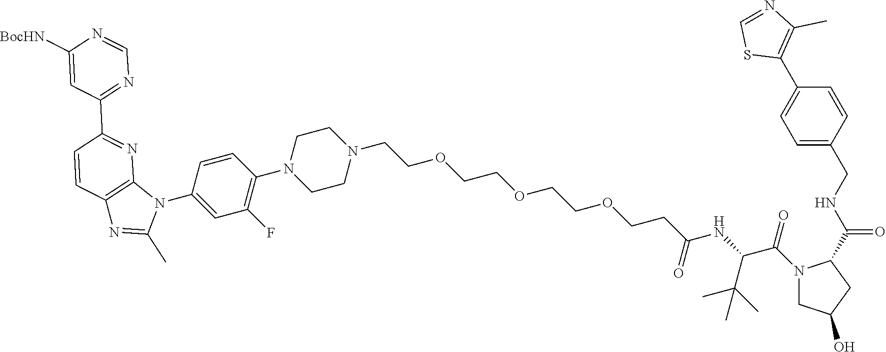 [SMILES: Cc1ncsc1-c1ccc(CNC(=O)[C@@H]2C[C@@H](O)CN2C(=O)[C@@H](NC(=O)CCOCCOCCOCCN2CCN(c3ccc(-n4c(C)nc5ccc(-c6cc(NC(=O)OC(C)(C)C)ncn6)nc54)cc3F)CC2)C(C)(C)C)cc1]

(2S,4R)-1-((S)-1-(4-(4-(5-(6-aminopyrimidin-4-yl)-2-methyl-3H-imidazo[4,5-b]pyridin-3-yl)-2-fluorophenyl)piperazin-1-yl)-14-(tert-butyl)-12-oxo-3,6,9-trioxa-13-azapentadecan-15-oyl)-4-hydroxy-N-(4-(4-methylthiazol-5-yl)benzyl)pyrrolidine-2-carboxamide (2) To a 5 mL MW equipped with a magnetic stir bar containing tert-butyl (4-(3-(3-fluoro-4-(4-((S)-14-((2S,4R)-4-hydroxy-2-((4-(4-methylthiazol-5-yl)benzyl)carbamoyl)pyrrolidine-1-carbonyl)-15,15-dimethyl-12-oxo-3,6,9-trioxa-13-azahexadecyl)piperazin-1-yl)phenyl)-2-methyl-3H-imidazo[4,5-b]pyridin-5-yl)pyridin-2-yl)carbamate (2e) (47 mg, 42 μmol, 1.0 eq.) in DCM (3.5 mL) at 0° C. was added trifluoroacetic acid (0.70 mL, 9.1 mmol, 220 eq.). The reaction mixture was stirred for 2 hours at room temperature where it was then concentrated under reduced pressure and purified by preparative high-performance liquid chromatography (HPLC) to give (2S,4R)-1-((S)-1-(4-(4-(5-(6-aminopyrimidin-4-yl)-2-methyl-3H-imidazo[4,5-b]pyridin-3-yl)-2-fluorophenyl)piperazin-1-yl)-14-(tert-butyl)-12-oxo-3,6,9-trioxa-13-azapentadecan-15-oyl)-4-hydroxy-N-(4-(4-methylthiazol-5-yl)benzyl)pyrrolidine-2-carboxamide (20 mg, 20 μmol, 47% yield), a white solid and cleavage product (2S,4R)-1-((S)-14-(tert-butyl)-1-hydroxy-12-oxo-3,6,9-trioxa-13-azapentadecan-15-oyl)-4-hydroxy-N-(4-(4-methylthiazol-5-yl)benzyl)pyrrolidine-2-carboxamide (2) (13 mg, 20 μmol, 49% yield). Product characterization: ¹H NMR (400 MHz, CDCl₃) δ 8.66 (s, 1H), 8.07 (d, J=8.3 Hz, 1H), 7.90 (d, J=6.1 Hz, 1H), 7.74 (d, J=8.3 Hz, 1H), 7.45 (t, J=6.0 Hz, 1H), 7.35 (s, 4H), 7.31 (s, 1H), 7.22-7.07 (m, 5H), 6.39 (bs, 2H), 4.72 (t, J=8.0 Hz, 1H), 4.57 (dd, J=15.0, 6.6 Hz, 1H), 4.52 (d, J=8.4 Hz, 2H), 4.34 (dd, J=15.0, 5.3 Hz, 1H), 4.03 (d, J=11.2 Hz, 1H), 3.78-3.70 (m, 4H), 3.70-3.57 (m, 10H), 3.30 (t, J=4.9 Hz, 4H), 2.94 (s, 4H), 2.86-2.82 (m, 1H), 2.60 (s, 3H), 2.51 (s, 3H), 2.52-2.42 (m, 3H), 2.16 (dd, J=13.6, 8.0 Hz, 1H), 1.26 (s, 1H), 0.95 (s, 9H). LCMS (ESI) C₅₃H₆₆FN₁₁O₇S requires 1019.48, found 1020 (M/Z+H).

### Example 3. Synthesis of (2S,4R)-1-((S)-1-(4-(4-(5-(2-aminopyridin-4-yl)-2-methyl-3H-imidazo[4,5-b]pyridin-3-yl)-2- fluorophenyl)piperazin-1-yl)-17-(tert-butyl)-15-oxo-3,6,9,12-tetraoxa-16-azaoctadecan-18-oyl)-4-hydroxy-N-(4-(4-methylthiazol-5-yl)benzyl)pyrrolidine-2-carboxamide (3)

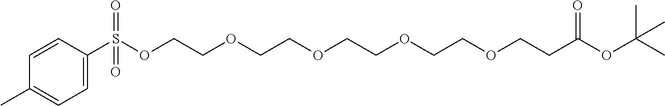 [SMILES: Cc1ccc(S(=O)(=O)OCCOCCOCCOCCOCCC(=O)OC(C)(C)C)cc1]

### tert-butyl 1-(tosyloxy)-3,6,9,12-tetraoxapentadecan-15-oate (3b)

4-toluenesulfonyl chloride (900 mg, 4.72 mmol, 1.0 eq.), silver oxide (1.53 g, 6.61 mmol, 1.4 eq.) and potassium iodide (212 mg, 1.27 mmol, 0.27 eq.) were added to a 250 mL RBF equipped with a stir bar containing 2,2′-((oxybis(ethane-2,1-diyl))bis(oxy))bis(ethan-1-ol) (1.39 mL, 8.03 mmol, 1.7 eq.) in DCM (47 mL). The reaction mixture was protected from light and stirred at room temperature for 1 hour where it was filtered through celite and washed with DCM. The filtrate was extracted with DCM (4×15 mL) and washed with water (10 mL). The organic layers were recombined, dried over sodium sulfate, and concentrated under reduced pressure to give 2-(2-(2-(2-hydroxyethoxy)ethoxy)ethoxy)ethyl 4-methylbenzenesulfonate (3a) and was used in the next step without further purification. To a suspension of NaH (215 mg, 5.38 mmol, 1.35 eq. 60% Wt.) in 1,4-dioxane (7.00 mL) was added 2-(2-(2-(2-hydroxyethoxy)ethoxy)ethoxy)ethyl 4-methylbenzenesulfonate (3a) (3.61 mL, 27.31 mmol) in 1,4-dioxane (2.00 mL) at 0° C. After 15 minutes at 0° C., tert-butyl acrylate (1.14 mL, 7.80 mmol, 1.0 eq.) was added and the mixture was stirred for 20 hours at room temperature. The reaction mixture was quenched with saturated ammonum chloride, extracted with DCM (3×25 mL), and washed with water and brine. The organic layers were recombined, dried over sodium sulfate, and concentrated under reduced pressure. The residue was purified by flash column chromatography to give tert-butyl 1-(tosyloxy)-3,6,9,12-tetraoxapentadecan-15-oate (3b) (346 mg, 0.72 mmol, 18% yield, eluting at 3% MeOH), a clear liquid. ¹H NMR (500 MHz, CDCl₃) δ 7.79 (d, J=8.2 Hz, 2H), 7.33 (d, J=8.0 Hz, 2H), 4.18-4.12 (m, 2H), 3.73-3.65 (m, 4H), 3.63-3.58 (m, 8H), 3.58-3.56 (m, 4H), 2.49 (t, J=6.6 Hz, 2H), 2.44 (s, 3H), 1.44 (s, 9H). ¹³C NMR (126 MHz, CDCl₃) δ 171.02, 144.90, 133.16, 129.94, 128.11, 80.62, 70.88, 70.75, 70.70, 70.65, 70.63, 70.49, 69.36, 68.80, 67.02, 36.39, 28.22, 21.76.

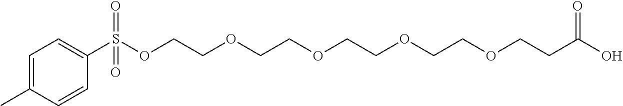 [SMILES: Cc1ccc(S(=O)(=O)OCCOCCOCCOCCOCCC(=O)O)cc1]

### 1-(tosyloxy)-3,6,9,12-tetraoxapentadecan-15-oic acid (3c)

TFA (1.12 mL, 14.5 mmol, 20 eq.) was added to a MWV equipped with a magnetic stir bar containing tert-butyl 1-(tosyloxy)-3,6,9,12-tetraoxapentadecan-15-oate (3b) (346 mg, 0.72 mmol, 1.0 eq.) and the reaction mixture was stirred for 2 hours at room temperature. The reaction mixture was diluted with DCM and concentrated under reduced pressure. The crude residue was purified by flash column chromatography to give 1-(tosyloxy)-3,6,9,12-tetraoxapentadecan-15-oic acid (3c) (239 mg, 0.56 mmol, 78% yield, eluting at 5% MeOH in DCM), a yellow oil. ¹H NMR (500 MHz, CDCl₃) δ 8.77 (s, 1H), 7.79 (d, J=8.2 Hz, 2H), 7.34 (d, J=8.0 Hz, 2H), 4.18-4.13 (m, 2H), 3.76 (t, J=6.2 Hz, 2H), 3.69 (dd, J=5.6, 4.0 Hz, 2H), 3.67-3.54 (m, 12H), 2.62 (t, J=6.1 Hz, 2H), 2.44 (s, 3H). ¹³C NMR (126 MHz, CDCl₃) δ 175.59, 144.96, 133.09, 129.96, 128.11, 70.85, 70.70, 70.68, 70.55, 70.53, 70.37, 69.36, 68.79, 66.47, 34.95, 21.76.

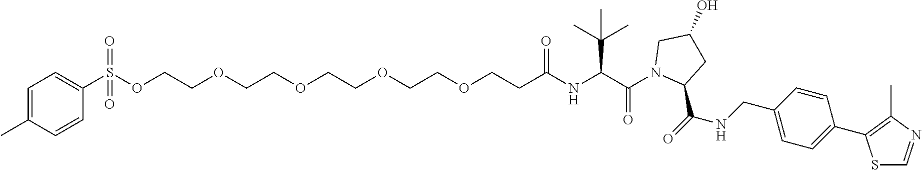 [SMILES: Cc1ccc(S(=O)(=O)OCCOCCOCCOCCOCCC(=O)N[C@H](C(=O)N2C[C@H](O)C[C@H]2C(=O)NCc2ccc(-c3scnc3C)cc2)C(C)(C)C)cc1]

### (S)-17-((2S,4R)-4-hydroxy-2-((4-(4-methylthiazol-5-yl)benzyl)carbamoyl)pyrrolidine-1-carbonyl)-18,18-dimethyl-15-oxo-3,6,9,12-tetraoxa-16-azanonadecyl 4-methylbenzenesulfonate (3d)

HATU (58 mg, 0.15 mmol, 1.1 eq.) was added to a 5 mL MWV containing 1-(tosyloxy)-3,6,9,12-tetraoxapentadecan-15-oic acid (3c) (61 mg, 0.14 mmol, 1.0 eq.), (2S,4R)-1-((S)-2-amino-3,3-dimethylbutanoyl)-4-hydroxy-N-(4-(4-methylthiazol-5-yl)benzyl)pyrrolidine-2-carboxamide (B6) (60 mg, 0.14 mmol, 1.0 eq.), DIPEA (0.15 mL, 0.84 mmol, 6.0 eq.) and DMF (1.20 mL). The reaction mixture was stirred at room temperature for 1 hour and was quenched with brine (15 ml) and extracted with DCM (3×15 ml). The organic layers were recombined and dried over sodium sulfate, concentrated under reduced pressure, and purified via flash column chromatography to give(S)-17-((2S,4R)-4-hydroxy-2-((4-(4-methylthiazol-5-yl)benzyl)carbamoyl)pyrrolidine-1-carbonyl)-18,18-dimethyl-15-oxo-3,6,9,12-tetraoxa-16-azanonadecyl 4-methylbenzenesulfonate (3d) (81 mg, 0.97 mmol, 70% yield, eluting at 7% MeOH in DCM), a white solid. ¹H NMR (500 MHz, CDCl₃) δ 8.70 (s, 1H), 7.78 (d, J=8.2 Hz, 2H), 7.40 (t, J=6.0 Hz, 1H), 7.38-7.30 (m, 6H), 6.99 (d, J=8.2 Hz, 1H), 4.72 (t, J=8.0 Hz, 1H), 4.54 (dd, J=14.9, 6.6 Hz, 1H), 4.50 (tt, J=4.1, 1.9 Hz, 1H), 4.44 (d, J=8.1 Hz, 1H), 4.33 (dd, J=15.0, 5.3 Hz, 1H), 4.17-4.12 (m, 2H), 4.09 (d, J=11.3 Hz, 1H), 3.73-3.64 (m, 4H), 3.65-3.53 (m, 12H), 2.54-2.41 (m, 9H), 2.16-2.07 (m, 1H), 1.49 (d, J=6.5 Hz, 1H), 1.42 (d, J=6.6 Hz, 1H), 0.93 (s, 9H). ¹³C NMR (126 MHz, CDCl₃) δ 172.31, 171.94, 170.95, 150.52, 148.36, 145.00, 138.40, 133.03, 130.88, 129.99, 129.60, 128.24, 128.09, 70.78, 70.59, 70.53, 70.48, 70.22, 69.38, 68.81, 67.25, 58.49, 57.95, 56.71, 54.55, 43.30, 36.73, 36.03, 34.85, 26.53, 21.77, 18.74, 17.45, 16.09.

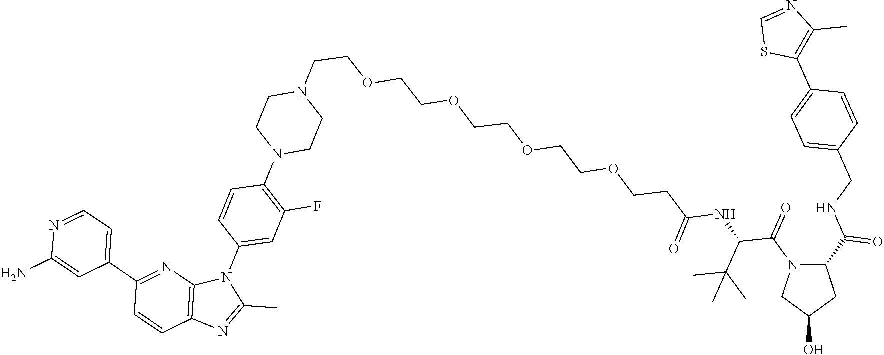 [SMILES: Cc1ncsc1-c1ccc(CNC(=O)[C@@H]2C[C@@H](O)CN2C(=O)[C@@H](NC(=O)CCOCCOCCOCCOCCN2CCN(c3ccc(-n4c(C)nc5ccc(-c6ccnc(N)c6)nc54)cc3F)CC2)C(C)(C)C)cc1]

### (2S,4R)-1-((S)-1-(4-(4-(5-(2-aminopyridin-4-yl)-2-methyl-3H-imidazo[4,5-b]pyridin-3-yl)-2-fluorophenyl)piperazin-1-yl)-17-(tert-butyl)-15-oxo-3,6,9,12-tetraoxa-16-azaoctadecan-18-oyl)-4-hydroxy-N-(4-(4-methylthiazol-5-yl)benzyl)pyrrolidine-2-carboxamide (3)

Acetonitrile (2.00 mL) was added to a 5 mL MWV equipped with a magnetic stir bar containing(S)-17-((2S,4R)-4-hydroxy-2-((4-(4-methylthiazol-5- yl)benzyl)carbamoyl)pyrrolidine-1-carbonyl)-18,18-dimethyl-15-oxo-3,6,9,12-tetraoxa-16-azanonadecyl 4-methylbenzenesulfonate (3d) (69 mg, 83 μmol, 1.0 eq.) and 4-(3-(3-fluoro-4-(piperazin-1-yl)phenyl)-2-methyl-3H-imidazo[4,5-b]pyridin-5-yl)pyridin-2-amine (A7) (32 mg, 79 μmol, 1.0 eq.), potassium carbonate (27 mg, 200 μmol, 2.5 eq.), and potassium iodide (2.2 mg, 13 μmol, 0.17 eq.). The resulting mixture was stirred for 16 hours at 80° C. where it was then extracted with DCM (3×15 mL) and washed with brine. The organic layers were recombined, dried over sodium sulfate, and concentrated under reduced pressure. The residue was purified by preparative HPLC to provide (2S,4R)-1-((S)-1-(4-(4-(5-(2-aminopyridin-4-yl)-2-methyl-3H-imidazo[4,5-b]pyridin-3-yl)-2-fluorophenyl)piperazin-1-yl)-17-(tert-butyl)-15-oxo-3,6,9,12-tetraoxa-16-azaoctadecan-18-oyl)-4-hydroxy-N-(4-(4-methylthiazol-5-yl)benzyl)pyrrolidine-2-carboxamide (3) (30 mg, 26 μmol, 33% yield), a yellow semisolid. ¹H NMR (500 MHz, CDCl₃) δ 8.67 (s, 1H), 8.30 (s, 4H), 8.15-8.07 (m, 1H), 7.82-7.68 (m, 3H), 7.46 (s, 1H), 7.39 (d, J=8.8 Hz, 1H), 7.37-7.27 (m, 4H), 7.05-6.87 (S, 2H), 4.69 (t, J=8.2 Hz, 1H), 4.60-4.49 (m, 3H), 4.32 (dd, J=15.2, 5.4 Hz, 1H), 4.00 (d, J=11.1 Hz, 1H), 3.94-3.87 (m, 2H), 3.68-3.56 (m, 15H),i 3.53-3.48 (m, 4H), 3.48-3.43 (m, 4H), 3.31-3.16 (m, 2H), 2.59 (s, 2H), 2.52-2.39 (m, 5H), 2.32 (ddd, J=13.1, 8.6, 4.2 Hz, 1H), 2.23 (dd, J=13.4, 7.9 Hz, 1H), 0.96 (s, 9H). ¹³C NMR (126 MHz, CDCl₃) δ 172.17, 171.67, 171.44, 166.36, 156.14, 156.06, 155.94, 152.27, 150.66, 149.02, 148.23, 146.29, 138.57, 136.79, 135.76, 131.95, 130.63, 129.47, 128.07, 127.28, 123.90, 120.08, 117.35, 109.97, 109.89, 70.58, 70.51, 70.39, 70.33, 70.11, 67.21, 65.98, 59.22, 57.89, 57.27, 56.55, 52.63, 47.67, 43.16, 36.99, 36.59, 35.58, 26.54, 15.98, 15.30. LCMS (ESI) C₅₅H₇₀FN₁₁O₈S requires 1063.51, found 1064 (M/Z+H).

### Example 4. Synthesis of (2S,4R)-1-((S)-1-(4-(4-(5-(2-aminopyridin-4-yl)-2-methyl-3H-imidazo[4,5-b]pyridin-3-yl)-2-fluorophenyl)piperazin-1-yl)-20-(tert-butyl)-18-oxo-3,6,9,12,15-pentaoxa-19-azahenicosan-21-oyl)-4-hydroxy-N-(4-(4-methylthiazol-5-yl)benzyl)pyrrolidine-2-carboxamide (4)

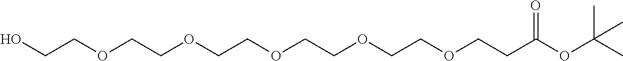 [SMILES: CC(C)(C)OC(=O)CCOCCOCCOCCOCCOCCO]

### tert-butyl 1-hydroxy-3,6,9,12,15-pentaoxaoctadecan-18-oate (4a)

To a suspension of sodium hydride (85 mg, 2.15 mmol, 1.1 eq., 60% Wt.) in 1,4-dioxne (3.35 mL) was added 3,6,9,12-tetraoxatetradecane-1,14-diol (1.24 mL, 5.85 mmol, 3.0 eq.) at 0° C. After 15 minutes, tert-butyl acrylate (0.28 mL, 1.95 mmol, 1.0 eq.) was added and the reaction mixture was warmed to room temperature and stirred for 20 hours. The reaction mixture was concentrated under reduced pressure, quenched with ammonium chloride, and extracted with DCM and washed with water. The organic layers were recombined, dried over sodium sulfate, and concentrated under reduced pressure. The residue was purified by flash column chromatography to give tert-butyl 1-hydroxy-3,6,9,12,15-pentaoxaoctadecan-18-oate (4a) (337 mg, 0.92 mmol, 47% yield), a clear oil. ¹H NMR (400 MHz, CDCl₃) δ 3.75-3.58 (m, 22H), 2.64 (bs, 1H), 2.51 (t, J=6.6 Hz, 2H), 1.46 (s, 9H). ¹³C NMR (101 MHz, CDCl₃) δ 171.03, 80.61, 77.48, 77.16, 76.84, 72.62, 70.73, 70.70, 70.67, 70.60, 70.47, 67.00, 61.85, 36.38, 28.20.

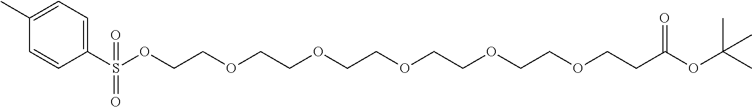 [SMILES: Cc1ccc(S(=O)(=O)OCCOCCOCCOCCOCCOCCC(=O)OC(C)(C)C)cc1]

### tert-butyl 1-(tosyloxy)-3,6,9,12,15-pentaoxaoctadecan-18-oate (4b)

To a solution of tert-butyl 1-hydroxy-3,6,9,12,15-pentaoxaoctadecan-18-oate (4a) (188 mg, 513 μmol, 1.0 eq.) in DCM (1.5 mL) were added 4-methylbenzenesulfonyl chloride (137 mg, 718 μmol, 1.4 eq.), triethylamine (0.08 mL, 564 μmol, 1.1 eq.), and DMAP (6 mg, 51 μmol, 0.1 eq.). The reaction mixture was stirred for 24 hours and was extracted with DCM (3×10 ml) and washed with water. The organic layers were recombined, dried over sodium sulfate, and concentrated under reduced pressure. The resulting crude residue was purified by flash column chromatography to give tert-butyl 1-(tosyloxy)-3,6,9,12,15-pentaoxaoctadecan-18-oate (4b) (197 mg, 378 μmol, 73% yield), a yellow oil. ¹H NMR (400 MHz, CDCl₃) δ 7.79 (d, J=8.2 Hz, 2H), 7.33 (d, J=8.0 Hz, 2H), 4.19-4.11 (m, 2H), 3.72-3.66 (m, 4H), 3.65-3.59 (m, 12H), 3.58-3.56 (m, 4H), 2.49 (td, J=6.6, 1.1 Hz, 2H), 2.44 (s, 3H), 1.44 (d, J=1.1 Hz, 9H). ¹³C NMR (101 MHz, CDCl₃) δ 171.01, 144.89, 133.17, 129.93, 128.10, 80.61, 77.48, 77.16, 76.84, 70.87, 70.74, 70.71, 70.69, 70.64, 70.62, 70.48, 69.35, 68.80, 67.02, 36.39, 28.21, 21.75.

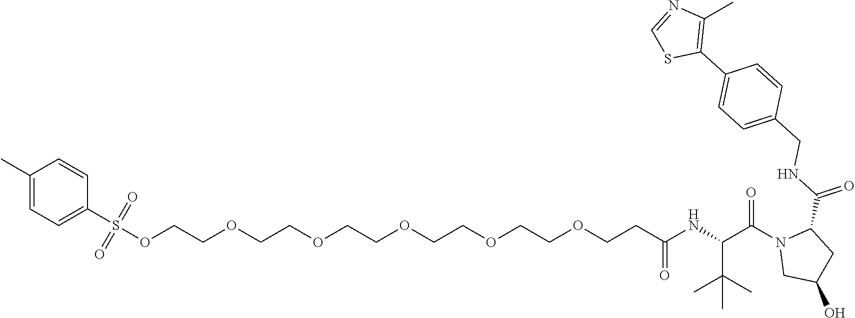 [SMILES: Cc1ccc(S(=O)(=O)OCCOCCOCCOCCOCCOCCC(=O)N[C@H](C(=O)N2C[C@H](O)C[C@H]2C(=O)NCc2ccc(-c3scnc3C)cc2)C(C)(C)C)cc1]

### (S)-20-((2S,4R)-4-hydroxy-2-((4-(4-methylthiazol-5-yl)benzyl)carbamoyl)pyrrolidine-1-carbonyl)-21,21-dimethyl-18-oxo-3,6,9,12,15-pentaoxa-19-azadocosyl 4-methylbenzenesulfonate (4d)

Trifluoroacetic acid (0.14 mL, 1.89 mmol, 5.0 eq.) was added to a solution of tert-butyl 1-(tosyloxy)-3,6,9,12,15-pentaoxaoctadecan-18-oate (197 mg, 0.37 mmol, 1.0 eq.) in DCM (1.40 mL). The reaction mixture was stirred for 2 hours at room temperature. The reaction mixture was diluted with DCM and concentrated under reduced pressure to give 1-(tosyloxy)-3,6,9,12,15-pentaoxaoctadecan-18-oic acid (4c) and was used in the next step without further purification. HATU (68 mg, 0.18 mmol, 1.1 eq.) was added to a 5 ml MWV containing 1-(tosyloxy)-3,6,9,12,15-pentaoxaoctadecan-18-oic acid (4c) (76 mg, 0.16 mmol, 1.0 eq), (2S,4R)-1-((S)-2-amino-3,3-dimethylbutanoyl)-4-hydroxy-N-(4-(4-methylthiazol-5-yl)benzyl)pyrrolidine-2-carboxamide (B6) (70 mg, 0.16 mmol, 1.0 eq.), DIPEA (0.17 mL, 0.98 mmol, 6.0 eq.) and DMF (1.30 mL). The reaction mixture was stirred at room temperature for 1 hour where the reaction was the quenched with brine and extracted with DCM (3×15 ml). The organic layer was dried over sodium sulfate, concentrated under reduced pressure, and purified via flash column chromatography to give(S)-20-((2S,4R)-4-hydroxy-2-((4-(4-methylthiazol-5-yl)benzyl)carbamoyl)pyrrolidine-1-carbonyl)-21,21-dimethyl-18-oxo-3,6,9,12,15-pentaoxa-19-azadocosyl 4-methylbenzenesulfonate (4d) (80 mg, 91 μmol, 56%), a clear oil. ¹H NMR (400 MHz, CDCl₃) δ 8.68 (s, 1H), 7.77 (d, J=8.0 Hz, 2H), 7.40 (t, J=5.9 Hz, 1H), 7.37-7.28 (m, 6H), 6.95 (d, J=8.2 Hz, 1H), 4.70 (t, J=7.9 Hz, 1H), 4.58-4.42 (m, 3H), 4.33 (dd, J=15.0, 5.3 Hz, 1H), 4.18-4.11 (m, 2H), 4.06 (d, J=11.2 Hz, 1H), 3.76-3.55 (m, 21H), 2.53-2.45 (m, 5H), 2.43 (s, 3H), 2.13 (t, J=10.9 Hz, 1H), 1.25 (s, 1H), 0.94 (s, 9H). ¹³C NMR (101 MHz, CDCl₃) δ 172.34, 171.81, 171.05, 150.49, 145.06, 138.36, 132.91, 130.90, 130.01, 129.56, 128.18, 128.05, 77.48, 77.16, 76.84, 70.58, 70.30, 70.26, 70.24, 70.22, 69.40, 68.74, 67.20, 58.60, 57.94, 56.71, 43.24, 36.51, 36.24, 34.96, 26.52, 21.76, 16.14. Note: Many peaks are superimposed on 70.3 ppm that can't be individually measured.

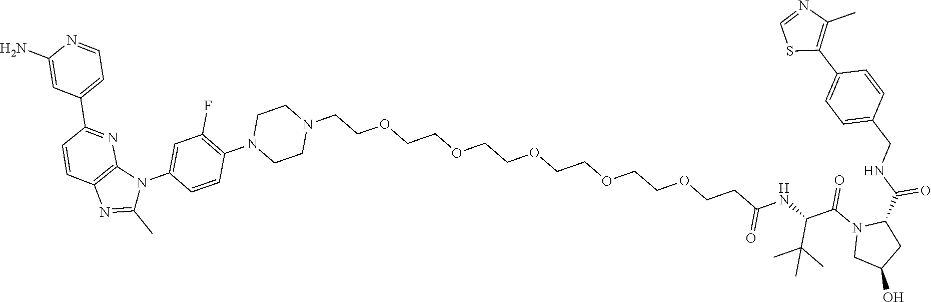 [SMILES: Cc1ncsc1-c1ccc(CNC(=O)[C@@H]2C[C@@H](O)CN2C(=O)[C@@H](NC(=O)CCOCCOCCOCCOCCOCCN2CCN(c3ccc(-n4c(C)nc5ccc(-c6ccnc(N)c6)nc54)cc3F)CC2)C(C)(C)C)cc1]

### (2S,4R)-1-((S)-1-(4-(4-(5-(2-aminopyridin-4-yl)-2-methyl-3H-imidazo[4,5-b]pyridin-3-yl)-2-fluorophenyl)piperazin-1-yl)-20-(tert-butyl)-18-oxo-3,6,9,12,15-pentaoxa-19-azahenicosan-21-oyl)-4-hydroxy-N-(4-(4-methylthiazol-5-yl)benzyl)pyrrolidine-2-carboxamide (4)

To a 5 mL MWV equipped with a magnetic stir bar containing(S)-20-((2S,4R)-4-hydroxy-2-((4-(4-methylthiazol-5-yl)benzyl)carbamoyl)pyrrolidine-1-carbonyl)-21,21-dimethyl-18-oxo-3,6,9,12,15-pentaoxa-19-azadocosyl 4-methylbenzenesulfonate (4d) (68 mg, 78 μmol, 1.0 eq.), 4-(3-(3-fluoro-4-(piperazin-1-yl)phenyl)-2-methyl-3H-imidazo[4,5-b]pyridin-5-yl)pyridin-2-amine (A7) (935 mg, 78 μmol, 1.0 eq.) and sodium iodide (2.9 mg, 20 μmol, 0.25 eq.) was added 1,4-dioxane (1.50 mL) and DIPEA (0.14 mL, 780 μmol, 10.0 eq.) sequentially. The reaction mixture was stirred at 90° C. for 10 hours. The reaction was concentrated under reduced pressure and purified via flash column chromatography using a DCM/MeOH with 3% triethylamine gradient to give (4) (2S,4R)-1-((S)-1-(4-(4-(5-(2-aminopyridin-4-yl)-2-methyl-3H-imidazo[4,5-b]pyridin-3-yl)-2-fluorophenyl)piperazin-1-yl)-20-(tert-butyl)-18-oxo-3,6,9,12,15-pentaoxa-19-azahenicosan-21-oyl)-4-hydroxy-N-(4-(4-methylthiazol-5-yl)benzyl)pyrrolidine-2-carboxamide (14 mg, 13 μmol, 17% yield, eluting at 19% MeOH in DCM), a clear semisolid. ¹H NMR (500 MHz, CDCl₃) δ 8.75 (s, 1H), 8.18-8.06 (m, 1H), 7.95-7.80 (m, 1H), 7.76-7.62 (m, 1H), 7.56-7.45 (m, 1H), 7.39-7.02 (m, 10H), 4.63-4.39 (m, 3H), 4.31 (d, J=15.2 Hz, 1H), 4.11-3.84 (m, 4H), 3.60-3.53 (m, 10H), 3.40-3.20 (m, 7H), 3.14-3.01 (m, 4H), 2.64-2.52 (m, 2H), 2.51-2.34 (m, 5H), 2.23-2.07 (m, 2H), 2.05-1.92 (m, 2H), 1.40-1.27 (m, 5H), 1.28-1.12 (m, 4H), 0.99-0.85 (m, 10H). ¹⁹F NMR (471 MHz, CDCl₃) δ -115.58.

### Example 5. Synthesis of tert-butyl (4-(3-(4-(4-(2-(2-(2-((2-(2,6-dioxopiperidin-3-yl)-1,3-dioxoisoindolin-4-yl)amino)ethoxy)ethoxy)ethyl)piperazin-1-yl)-3-fluorophenyl)-2-methyl-3H-imidazo[4,5-b]pyridin-5-yl)pyridin-2-yl)carbamate (5)

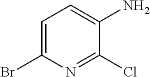

### 6-bromo-2-chloropyridin-3-amine (A1)

NBS (6.092 g, 1.1 Eq, 34.23 mmol) was added over 15 minutes to a dry RBF containing 2-chloropyridin-3-amine (4.000 g, 1 Eq, 31.11 mmol) and DCM (104 mL). The reaction mixture was stirred at room temperature for 18 hours. The reaction mixture was diluted with DCM and washed with started sodium bicarbonate and extracted with DCM (3×30 ml). The organic layer was washed with 10% NaOH, H₂O, brine, and the organic layer was dried over Na₂SO₄, concentrated under reduced pressure to give 6-bromo-2-chloropyridin-3-amine (A1) (6.13 g, 29.56 mmol, 95%) and was used without further purification. ¹H NMR (500 MHz, CDCl₃) δ 7.22-7.13 (m, 1H), 6.94 (d, J=8.2 Hz, 1H), 4.16 (d, J=17.1 Hz, 2H).

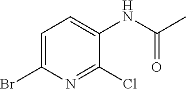

### N-(6-bromo-2-chloropyridin-3-yl)acetamide (A2)

Ac₂O (37.3 mL, 395 mmol, 12.7 eq.) was added dropwise to a dry RBF containing 6-bromo-2-chloropyridin-3-amine (A1) (6.40 g, 31.11 mmol, 1.0 eq.) and acetic acid (35.6 mL, 20.0 Eq, 622 mmol). The reaction mixture was sealed and stirred at room temperature for 18 hours. The mixture was extracted with ethyl acetate (5×30 ml) and washed with an aqueous solution of 10% K₂CO₃ and brine. K₂CO₃ is added until there is no more bubbling. The organic layers were recombined, dried over sodium sulfate, and concentrated under reduced pressure. The black liquid was azeotroped with toluene and hexanes repeatedly until the residue was a dry solid. This was purified by flash column chromatography to give N-(6-bromo-2-chloropyridin-3-yl)acetamide (A2) (6.54 g, 26.21 mmol, 84%), a red solid. ¹H NMR (500 MHz, CDCl₃) δ 8.65-8.58 (m, 1H), 7.59 (s, 1H), 7.40 (d, J=8.6 Hz, 1H), 2.26 (s, 3H).

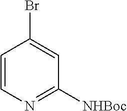 [SMILES: CC(C)(C)OC(=O)Nc1cc(Br)ccn1]

### tert-butyl (4-bromopyridin-2-yl)carbamate (A3)

A solution of 4-bromopyridin-2-amine (5.00 g, 28.90 mmol, 1.0 eq.) in dry THF (100 mL) was treated with LiHMDS (57.8 mL, 57.80 mmol, 1.0 M, 2.0 eq.) at −5° C. and the solution as stirred for 10 minutes. Boc₂O (6.64 mL, 28.90 mmol, 1.0 eq.) was added and the mixture was allowed to warm to room temperature and stirred for 2 hours. Upon completion, the reaction mixture was quenched with saturated ammonium chloride solution (20 mL) and extracted with ethyl acetate (3×60 mL). The combined organic layers were washed with brine (20 mL), dried with sodium sulfate, filtered, and concentrated under reduced pressure. The crude residue was purified by lash column chromatography then washed with hexanes to remove yellow oil impurity to give tert-butyl (4-bromopyridin-2-yl)carbamate (A3) (5.49 g, 20.13 mmol, 69%), a crystalline white solid.

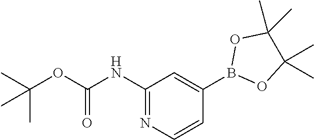 [SMILES: CC(C)(C)OC(=O)Nc1cc(B2OC(C)(C)C(C)(C)O2)ccn1]

### tert-butyl (4-(4,4,5,5-tetramethyl-1,3,2-dioxaborolan-2-yl)pyridin-2-yl)carbamate (A4)

To a batch of five 20 ml microwave vials equipped with a magnetic stir bar were added equally tert-butyl (4-bromopyridin-2-yl)carbamate (A3) (4.32 g, 15.8 mmol, 1.0 eq.), 4,4,4′,4′,5,5,5′,5′-octamethyl-2,2′-bi (1,3,2-dioxaborolane) (5.62 g, 22.1 mmol, 1.4 eq.), potassium acetate (4.97 g, 50.6 mmol, 3.2 eq.), PdCl2 (dppf) (926 mg, 1.27 mmol, 0.08 eq.), and degassed 1,4-Dioxane (78 mL). The MWV's were purged with argon and heated at 90° C. for 3 hours in an oil bath. The reaction vessels were cooled, diluted with EtOAc, and all poured into a separatory funnel. The mixture was extracted and washed with water/brine (3×). The organic layers were recombined, dried over sodium sulfate, filtered, and concentrated under reduced pressure. The crude residue was washed with cold ethanol and filtered to give tert-butyl (4-(4,4,5,5-tetramethyl-1,3,2-dioxaborolan-2-yl)pyridin-2-yl)carbamate (A4) (4.60 g, 14.37 mmol, 90% yield), a grey solid.

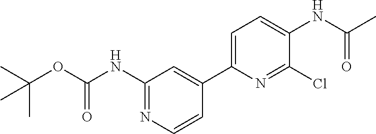 [SMILES: CC(=O)Nc1ccc(-c2ccnc(NC(=O)OC(C)(C)C)c2)nc1Cl]

### tert-butyl (5-acetamido-6-chloro-[2,4′-bipyridin]-2′-yl)carbamate (A5)

N-(6-bromo-2-chloropyridin-3-yl)acetamide (3.30 g, 13.23 mmol, 1.1 eq.), tert-butyl (4-(4,4,5,5-tetramethyl-1,3,2-dioxaborolan-2-yl)pyridin-2-yl)carbamate (A4) (4.640 g, 12.03 mmol, 1.0 eq., 83% Wt.), PdCl2 (dppf) (880 mg, 1.20 mmol, 0.1 eq.), and tripotassium phosphate (5.616 g, 26.46 mmol, 2.2 eq.) were added to a flame dried 250 mL RBF equipped with a magnetic stir bar. The RBF was capped, purged with argon, then injected with degassed dioxane: H₂O (4:1, 60 mL) and was heated at 90° C. for 5 hours in an oil bath. The reaction was cooled, diluted with DCM, filtered through celite and washed with a solution of 5% MeOH in DCM. The filtrate was concentrated under reduced pressure and purified by flash column chromatography to give tert-butyl (5-acetamido-6-chloro-[2,4′-bipyridin]-2′-yl)carbamate (A5) (2.70 g, 7.44 mmol, 61%, eluting at 4% MeOH in DCM) as a beige solid. ¹H NMR (500 MHz, DMSO) δ 9.91 (s, 1H), 9.80 (s, 1H), 8.44 (d, J=1.6 Hz, 1H), 8.41 (d, J=8.3 Hz, 1H), 8.36 (d, J=5.2 Hz, 1H), 8.06 (d, J=8.4 Hz, 1H), 7.61 (dd, J=5.3, 1.6 Hz, 1H), 2.18 (s, 3H), 1.51 (s, 9H). LCMS (ESI) C₁₇H₁₉ClN₄O₃ requires 362.11, found 363 (M/Z+H).

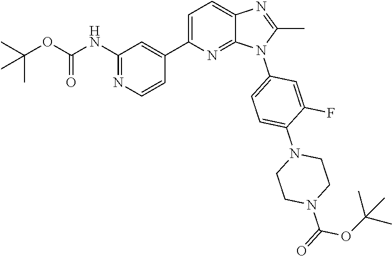 [SMILES: Cc1nc2ccc(-c3ccnc(NC(=O)OC(C)(C)C)c3)nc2n1-c1ccc(N2CCN(C(=O)OC(C)(C)C)CC2)c(F)c1]

### tert-butyl 4-(4-(5-(2-((tert-butoxycarbonyl)amino)pyridin-4-yl)-2-methyl-3H-imidazo[4,5-b]pyridin-3-yl)-2-fluorophenyl)piperazine-1-carboxylate (A6)

Potassium phosphate (3.51 g, 16.53 mmol, 3.0 eq.), tert-butyl (5-acetamido-6-chloro-[2,4′-bipyridin]-2′-yl)carbamate (A5) (2.00 g, 5.51 mmol, 1.0 eq.), tert-butyl 4-(4-amino-2-fluorophenyl)piperazine-1-carboxylate (1.79 g, 6.06 mmol, 1.1 eq.), dicyclohexyl(2′,4′,6′-triisopropyl-[1,1′-biphenyl]-2-yl)phosphane (262 mg, 0.55 mmol, 0.10 eq.), and Pd2(dba)3 (100.96 mg, 0.11 mmol, 0.02 eq.) were added to a flame-dried 5 ml MWV equipped with a magnetic stir bar. The MWV was capped and purged with argon where degassed t-BuOH (14.00 mL) was added and heated to 90° C. for 24 hours. The reaction mixture was allowed to cool to room temperature and diluted with dichloromethane. The diluted mixture was filtered through Celite and. The filtrate was concentrated under reduced pressure and purified by flashcolumn chromatography to give tert-butyl 4-(4-(5-(2-((tert-butoxycarbonyl)amino)pyridin-4-yl)-2-methyl-3H-imidazo[4,5-b]pyridin-3-yl)-2-fluorophenyl)piperazine-1-carboxylate (A6) (2.17 g, 3.60 mmol, 65%, eluting at 5% MeOH in DCM), a brown solid. ¹H NMR (500 MHz, DMSO) δ 9.76 (s, 1H), 8.47 (s, 1H), 8.30 (d, J=5.3 Hz, 1H), 8.15 (d, J=8.2 Hz, 1H), 7.94 (d, J=8.3 Hz, 1H), 7.63-7.55 (m, 2H), 7.43 (dd, J=8.6, 2.4 Hz, 1H), 7.27 (t, J=9.0 Hz, 1H), 3.53 (t, J=4.9 Hz, 4H), 3.11 (t, J=5.1 Hz, 4H), 2.55 (s, 3H), 1.47 (s, 9H), 1.45 (s, 9H).

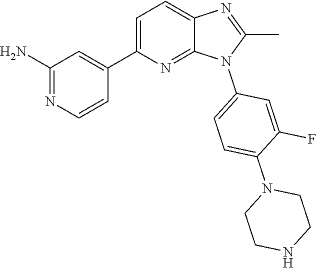 [SMILES: Cc1nc2ccc(-c3ccnc(N)c3)nc2n1-c1ccc(N2CCNCC2)c(F)c1]

4-(3-(3-fluoro-4-(piperazin-1-yl)phenyl)-2-methyl-3H-imidazo[4,5-b]pyridin-5-yl)pyridin-2-amine (A7)

TFA (6.28 mL, 82.8 mmol, 20.0 eq.) was added to a solution of tert-butyl 4-(4-(5-(2-((tert-butoxycarbonyl)amino)pyridin-4-yl)-2-methyl-3H-imidazo[4,5-b]pyridin-3-yl)-2-fluorophenyl)piperazine-1-carboxylate (A6) (2.50 g, 4.14 mmol, 1.0 eq.) in DCM (21 mL) and was stirred at room temperature for 16 hours. The reaction mixture was neutralized with 1.0 M NaOH, and extracted with DCM. The combined organic layers were washed with water, dried over sodium sulfate, and concentrated under reduced pressure. The crude residue was purified by flash column chromatography to give 4-(3-(3-fluoro-4-(piperazin-1-yl)phenyl)-2-methyl-3H-imidazo[4,5-b]pyridin-5-yl)pyridin-2-amine (A7) (1.24 g, 3.08 mmol, 74%, eluting at 15% MeOH in DCM), a light-brown solid. ¹H NMR (500 MHz, CDCl₃) δ 8.10 (d, J=5.5 Hz, 1H), 8.03 (d, J=8.2 Hz, 1H), 7.70 (d, J=8.3 Hz, 1H), 7.23-7.14 (m, 3H), 7.12-7.06 (m, 2H), 4.56 (s, 2H), 3.18 (dd, J=6.4, 3.3 Hz, 4H), 3.10 (dd, J=6.2, 3.4 Hz, 4H), 2.57 (s, 3H), 1.29-1.21 (m, 1H). ¹³C NMR (126 MHz, MeOD) δ 155.25 (d, J=248.3 Hz), 139.60 (d, J=8.4 Hz), 129.02 (d, J=10.3 Hz), 124.17 (d, J=3.5 Hz), ¹³C NMR (126 MHz, MeOD) δ 166.2, 156.4, 156.3, 155.25 (d, J=248.3 Hz), 152.0, 148.8, 146.7, 139.60 (d, J=8.4 Hz), 138.4, 135.1, 129.0 (d, J=10.3 Hz), 126.4, 124.1 (d, J=3.5 Hz), 119.89 (d, J=3.2 Hz), 117.15, 115.84 (d, J=23.4 Hz), 110.21, 108.54, 48.46, 43.52, 13.56. LCMS (ESI) C₂₂H₂₂FN₇ requires 403.47, found 404 (M/Z+H).

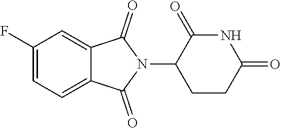 [SMILES: O=C1CCC(N2C(=O)c3ccc(F)cc3C2=O)C(=O)N1]

### 2-(2,6-dioxopiperidin-3-yl)-5-fluoroisoindoline-1,3-dione (C1)

Acetic acid (50.00 mL) was added to a 250 mL RBF equipped with a magnetic stir bar containing sodium acetate (1.23 g, 15.05 mmol, 1.0 eq.), 5-fluoroisobenzofuran-1,3-dione (2.50 g, 15.05 mmol, 1.0 eq.), 3-aminopiperidine-2,6-dione, HCl (2.47 g, 15.05 mmol, 1.0 eq.), and was stirred at 120° C. overnight, cooled, and concentrated under reduced pressure. The residue was suspended in D.I. water (100 mL) and stirred for 3 hours where it was then filtered over a fritted funnel. The solid was dissolved in methanol, concentrated under reduced pressure, washed with hexanes, and filtered to give 2-(2,6-dioxopiperidin-3-yl)-5-fluoroisoindoline-1,3-dione (C) (3.74 g, 13.57 mmol, 90% yield), a purple solid. ¹H NMR (500 MHz, DMSO) δ 11.15 (s, 1H), 8.01 (dd, J=8.3, 4.5 Hz, 1H), 7.85 (dd, J=7.4, 2.4 Hz, 1H), 7.73 (td, J=8.9, 2.4 Hz, 1H), 5.17 (dd, J=13.0, 5.4 Hz, 1H), 2.90 (ddd, J=17.1, 13.9, 5.4 Hz, 1H), 2.66-2.52 (m, 2H), 2.08 (dtd, J=13.1, 5.4, 2.3 Hz, 1H). ¹³C NMR (126 MHz, DMSO) δ 172.77, 169.77, 166.21, 166.01 (d, J=254.2 Hz), 165.92 (d, J=3.2 Hz), 134.22 (d, J=9.8 Hz), 127.44 (d, J=2.6 Hz), 126.30 (d, J=9.7 Hz), 121.78 (d, J=23.7 Hz), 111.45 (d, J=25.2 Hz), 49.21, 30.93, 21.95.

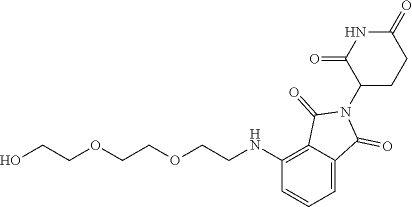 [SMILES: O=C1CCC(N2C(=O)c3cccc(NCCOCCOCCO)c3C2=O)C(=O)N1]

### 2-(2,6-dioxopiperidin-3-yl)-4-((2-(2-(2-hydroxyethoxy)ethoxy)ethyl)amino)isoindoline-1,3-dione (5a)

To a 5 mL MWV equipped with a magnetic stir bar was added 2-(2-(2-aminoethoxy)ethoxy)ethan-1-ol (0.16 mL, 1.18 mmol, 1.0 eq.), DIPEA (1.23 mL, 7.08 mmol, 6.0 eq.) and 1,4-dioxane (3.6 mL). This mixture was stirred for 5 minutes at room temperature under an argon atmosphere where 2-(2,6-dioxopiperidin-3-yl)-4-fluoroisoindoline-1,3-dione (C1) (391 mg, 1.42 mmol, 1.2 eq.) was added and the reaction mixture was stirred at 115° C. for 6 days. The reaction mixture was cooled to room temperature, concentrated under reduced pressure and purified by flash column chromatography to give 2-(2,6-dioxopiperidin-3-yl)-4-((2-(2-(2-hydroxyethoxy)ethoxy)ethyl)amino)isoindoline-1,3-dione (5a) (272 mg, 0.67 mmol, 56% yield), a brown semisolid. ¹H NMR (500 MHz, CDCl₃) δ 8.41 (s, 1H), 7.48 (dd, J=8.5, 7.1 Hz, 1H), 7.09 (d, J=7.1 Hz, 1H), 6.90 (d, J=8.5 Hz, 1H), 6.55 (s, 1H), 4.95-4.87 (m, 1H), 3.77-3.70 (m, 4H), 3.68 (s, 4H), 3.66-3.56 (m, 3H), 3.49-3.44 (m, 2H), 2.90-2.83 (m, 1H), 2.83-2.67 (m, 2H), 2.14-2.07 (m, 1H).

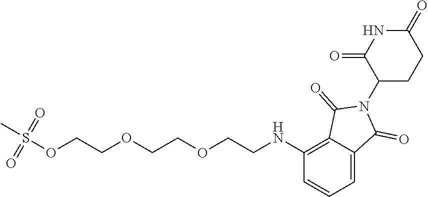 [SMILES: CS(=O)(=O)OCCOCCOCCNc1cccc2c1C(=O)N(C1CCC(=O)NC1=O)C2=O]

### 2-(2-(2-((2-(2,6-dioxopiperidin-3-yl)-1,3-dioxoisoindolin-4-yl)amino)ethoxy)ethoxy)ethyl methanesulfonate (5b)

In a 20 mL MWV equipped with a magnetic stir bar containing 2-(2,6-dioxopiperidin-3-yl)-4-((2-(2-(2-hydroxyethoxy)ethoxy)ethyl)amino)isoindoline-1,3-dione (5a) (247 mg, 609 μmol, 1.0 eq.), triethylamine (185 mg, 1.83 mmol, 3.0 eq.), in DCM (11.9 mL) was added methanesulfonyl chloride (0.06 mL, 731 μmol, 1.2 eq.) at 0° C. The reaction mixture was allowed to warm up to room temperature and was stirred for 24 hours. The reaction mixture was diluted with water and extracted with DCM (3×20 mL). The organic layers were recombined, dried over sodium sulfate, and concentrated under reduced pressure. The crude residue was purified by flash column chromatography using a DCM/MeOH gradient to give 2-(2-(2-((2-(2,6-dioxopiperidin-3-yl)-1,3-dioxoisoindolin-4-yl)amino)ethoxy)ethoxy)ethyl methanesulfonate (5b) (223 mg, 0.46 mmol, 75% yield, eluting at 3% MeOH in DCM), a yellow semisolid. ¹H NMR (500 MHz, CDCl₃) δ 8.64 (s, 1H), 7.49 (dd, J=8.5, 7.1 Hz, 1H), 7.09 (d, J=7.1 Hz, 1H), 6.90 (d, J=8.6 Hz, 1H), 6.50 (d, J=5.8 Hz, 1H), 4.99-4.90 (m, 1H), 4.41-4.33 (m, 2H), 3.83-3.74 (m, 2H), 3.75-3.64 (m, 6H), 3.46 (q, J=4.8 Hz, 2H), 3.04 (s, 3H), 2.89-2.68 (m, 3H), 2.17-2.06 (m, 1H). ¹³C NMR (126 MHz, CDCl₃) δ 171.60, 169.41, 168.83, 167.68, 146.79, 136.15, 132.59, 116.79, 111.73, 110.38, 70.81, 70.57, 69.44, 69.38, 69.18, 48.94, 42.34, 37.58, 31.44, 22.83.

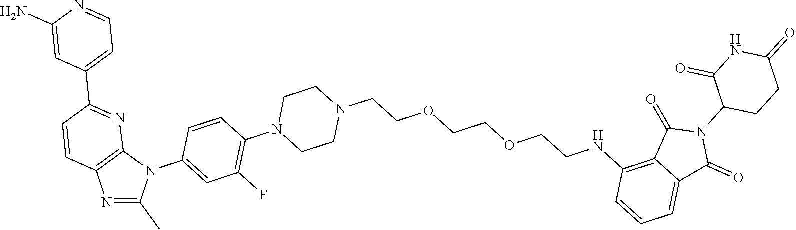 [SMILES: Cc1nc2ccc(-c3ccnc(N)c3)nc2n1-c1ccc(N2CCN(CCOCCOCCNc3cccc4c3C(=O)N(C3CCC(=O)NC3=O)C4=O)CC2)c(F)c1]

### tert-butyl (4-(3-(4-(4-(2-(2-(2-((2-(2,6-dioxopiperidin-3-yl)-1,3-dioxoisoindolin-4-yl)amino)ethoxy)ethoxy)ethyl)piperazin-1-yl)-3-fluorophenyl)-2-methyl-3H-imidazo[4,5-b]pyridin-5-yl)pyridin-2-yl)carbamate (5)

To a 5 mL MWV equipped with a magnetic stir bar containing 2-(2-(2-((2-(2,6-dioxopiperidin-3-yl)-1,3-dioxoisoindolin-4-yl)amino)ethoxy)ethoxy)ethyl methanesulfonate (5b) (50 mg, 100 μmol, 1.2 eq 4-(3-(3-fluoro-4-(piperazin-1-yl)phenyl)-2-methyl-3H-imidazo[4,5-b]pyridin-5-yl)pyridin-2-amine (A7) (35 mg, 87 μmol, 1.0 eq.), and sodium iodide (2.6 mg, 17 μmol, 0.2 eq.) was added DMF (1.75 mL) and DIPEA (0.15 mL, 17 μmol, 3.0 eq.) sequentially. The reaction mixture was stirred at 80° C. for 16 hours where the reaction mixture was extracted with DCM (3×10 mL) and washed with brine (10 mL). The organic layers were recombined, dried over sodium sulfate, concentrated under reduced pressure, and purified by flash column chromatography to give (5) 4-((2-(2-(2-(4-(4-(5-(2-aminopyridin-4-yl)-2-methyl-3H-imidazo[4,5-b]pyridin-3-yl)-2-fluorophenyl)piperazin-1-yl)ethoxy)ethoxy)ethyl)amino)-2-(2,6-dioxopiperidin-3-yl)isoindoline-1,3-dione (5) (24 mg, 87 μmol, 33% yield, 92% purity), a yellow semisolid. ¹H NMR (500 MHz, CDCl₃) δ 8.06 (dd, J=11.7, 6.8 Hz, 2H), 7.71 (d, J=8.2 Hz, 1H), 7.48 (dd, J=8.5, 7.1 Hz, 1H), 7.24-7.12 (m, 4H), 7.12-7.03 (m, 2H), 6.91 (d, J=8.5 Hz, 1H), 6.54 (t, J=5.6 Hz, 1H), 5.06 (s, 2H), 4.90-4.83 (m, 1H), 3.86-3.76 (m, 1H), 3.74 (t, J=5.3 Hz, 2H), 3.72-3.63 (m, 5H), 3.51-3.44 (m, 2H), 3.36-3.28 (m, 4H), 3.09 (q, J=7.4 Hz, 1H), 2.95 (s, 3H), 2.88-2.63 (m, 6H), 2.57 (s, 3H), 2.14-2.06 (m, 1H). LCMS (ESI) C₄₁H₄₃FN₁₀O₆ requires 790.86, found 791 (M/Z+H).

### Example 6. Synthesis of 4-((2-(2-(2-(2-(4-(4-(5-(2-aminopyridin-4-yl)-2-methyl-3H-imidazo[4,5-b]pyridin-3-yl)-2-fluorophenyl)piperazin-1-yl)ethoxy)ethoxy)ethoxy)ethyl)amino)-2-(2,6-dioxopiperidin-3-yl)isoindoline-1,3-dione (6)

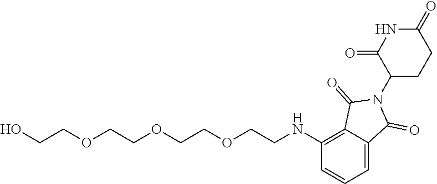 [SMILES: O=C1CCC(N2C(=O)c3cccc(NCCOCCOCCOCCO)c3C2=O)C(=O)N1]

### 2-(2,6-dioxopiperidin-3-yl)-4-((2-(2-(2-(2-hydroxyethoxy)ethoxy)ethoxy)ethyl)amino)isoindoline-1,3-dione (6a)

To a 5 mL MWV equipped with a magnetic stir bar was added 2-(2-(2-(2-aminoethoxy)ethoxy)ethoxy)ethan-1-ol (0.23 mL, 1.29 mmol, 1.0 eq.), DIPEA (1.35 mL, 7.76 mmol, 6.0 eq.), and 1,4-dioxane (3.6 mL). This was stirred for 5 minutes at room temperature under an argon atmosphere where 2-(2,6-dioxopiperidin-3-yl)-4-fluoroisoindoline-1,3-dione (C1) (429 mg, 1.55 mmol, 1.2 eq.) was added and the reaction mixture was stirred at 115° C. for 16 hours. The reaction mixture was cooled to room temperature, concentrated under reduced pressure, and purified by flash column chromatography to give 2-(2,6-dioxopiperidin-3-yl)-4-((2-(2-(2-(2-hydroxyethoxy)ethoxy)ethoxy)ethyl)amino)isoindoline-1,3-dione (6a) (206 mg, 0.45 mmol, 25% yield), a green semisolid. ¹H NMR (400 MHz, CDCl₃) δ 8.33-8.28 (m, 1H), 7.49 (dd, J=8.5, 7.1 Hz, 1H), 7.10 (d, J=7.1 Hz, 1H), 6.92 (d, J=8.5 Hz, 1H), 4.92 (dd, J=11.9, 5.4 Hz, 1H), 3.72 (t, J=4.8 Hz, 4H), 3.68 (d, J=1.6 Hz, 8H), 3.64-3.54 (m, 3H), 3.48 (t, J=5.4 Hz, 2H), 2.92-2.66 (m, 4H), 2.18-2.08 (m, 1H). ¹³C NMR (101 MHz, CDCl₃) δ 171.27, 169.42, 168.54, 167.75, 146.97, 136.19, 132.68, 116.98, 111.84, 110.50, 72.58, 70.92, 70.83, 70.73, 70.51, 69.61, 61.87, 49.00, 42.55, 31.53, 22.97.

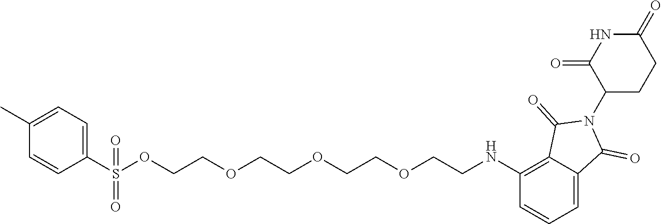 [SMILES: Cc1ccc(S(=O)(=O)OCCOCCOCCOCCNc2cccc3c2C(=O)N(C2CCC(=O)NC2=O)C3=O)cc1]

### 2-(2-(2-(2-((2-(2,6-dioxopiperidin-3-yl)-1,3-dioxoisoindolin-4-yl)amino)ethoxy)ethoxy)ethoxy)ethyl 4-methylbenzenesulfonate (6b)

In a 20 ml MWV equipped with a stir bar containing 2-(2,6-dioxopiperidin-3-yl)-4-((2-(2-(2-(2-hydroxyethoxy)ethoxy)ethoxy)ethyl)amino)isoindoline-1,3-dione (6a) (206 mg, 0.45 mmol, 1.0 eq.), triethylamine (0.19 mL, 1.37 mmol, 3.0 eq.), in DCM (9.00 mL) was added 4-toluenesulfonyl chloride (105 mg, 0.55 mmol, 1.2 eq.) at 0° C. The reaction mixture was stirred at room temperature for 72 hours where it was then diluted with water and extracted with DCM (3×15 ml). The organic layers were recombined, dried over sodium sulfate, concentrated under reduced pressure, and purified by flash column chromatography to give 2-(2-(2-(2-((2-(2,6-dioxopiperidin-3-yl)-1,3-dioxoisoindolin-4-yl)amino)ethoxy)ethoxy)ethoxy)ethyl 4-methylbenzenesulfonate (6b) (210 mg, 0.34 mmol, 75% yield), a yellow oil. ¹H NMR (400 MHz, CDCl₃) δ 8.22 (s, 1H), 7.80 (d, J=8.3 Hz, 2H), 7.50 (dd, J=8.5, 7.1 Hz, 1H), 7.35 (d, J=8.2 Hz, 2H), 7.11 (d, J=7.1 Hz, 1H), 6.94 (d, J=8.5 Hz, 1H), 4.99-4.90 (m, 1H), 4.21-4.13 (m, 2H), 3.77-3.69 (m, 4H), 3.69-3.58 (m, 8H), 3.49 (t, J=5.4 Hz, 2H), 2.95-2.69 (m, 3H), 2.46 (s, 3H), 2.21-2.10 (m, 1H). ¹H NMR (400 MHz, CDCl₃) δ 8.19 (s, 1H), 7.78 (d, J=8.3 Hz, 2H), 7.48 (dd, J=8.5, 7.1 Hz, 1H), 7.33 (d, J=8.2 Hz, 2H), 7.09 (d, J=7.1 Hz, 1H), 6.92 (d, J=8.5 Hz, 1H), 4.97-4.88 (m, 1H), 4.18-4.11 (m, 2H), 3.75-3.67 (m, 4H), 3.67-3.55 (m, 8H), 3.47 (t, J=5.4 Hz, 2H), 2.92-2.67 (m, 3H), 2.43 (s, 3H), 2.19-2.07 (m, 1H). ¹³C NMR (101 MHz, CDCl₃) δ 171.21, 169.38, 168.49, 167.74, 146.96, 144.93, 136.17, 133.12, 132.64, 129.95, 128.10, 116.95, 111.77, 110.42, 77.48, 77.16, 76.84, 70.89, 70.84, 70.78, 70.75, 69.63, 69.43, 68.82, 48.99, 42.55, 31.52, 22.89, 21.75.

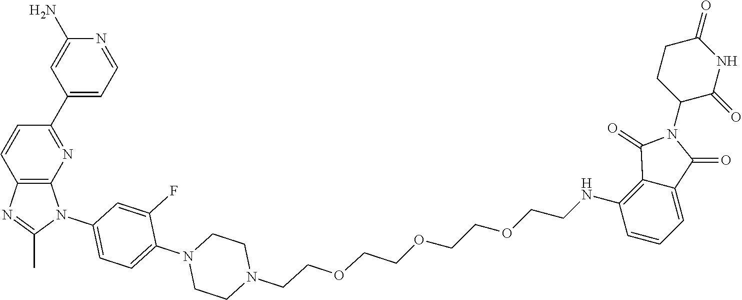 [SMILES: Cc1nc2ccc(-c3ccnc(N)c3)nc2n1-c1ccc(N2CCN(CCOCCOCCOCCNc3cccc4c3C(=O)N(C3CCC(=O)NC3=O)C4=O)CC2)c(F)c1]

### 4-((2-(2-(2-(2-(4-(4-(5-(2-aminopyridin-4-yl)-2-methyl-3H-imidazo[4,5-b]pyridin-3-yl)-2-fluorophenyl)piperazin-1-yl)ethoxy)ethoxy)ethoxy)ethyl)amino)-2-(2,6-dioxopiperidin-3-yl)isoindoline-1,3-dione (6)

To a 5 mL MWV equipped with a magnetic stir bar containing 4-(3-(3-fluoro-4-(piperazin-1-yl)phenyl)-2-methyl-3H-imidazo[4,5-b]pyridin-5-yl)pyridin-2-amine (A7) (24 mg, 59 μmol, 1.0 eq.), 2-(2-(2-(2-((2-(2,6-dioxopiperidin-3-yl)-1,3-dioxoisoindolin-4-yl)amino)ethoxy)ethoxy)ethoxy)ethyl 4-methylbenzenesulfonate (6b) (43 mg, 71 μmol, 1.2 eq.), and sodium iodide (2.2 mg, 15 μmol, 0.25 eq.) were added 1,4-dioxane (1.10 mL) and DIPEA (0.10 mL, 590 μmol, 10.0 eq.) sequentially. The reaction mixture was stirred at 90° C. for 10 hours where it was then extracted with DCM (3×10 mL) and washed with saturated ammonium chloride and water. The organic layers were recombined, dried over sodium sulfate, concentrated under reduced pressure, and purified by flash column chromatography to give 4-((2-(2-(2-(2-(4-(4-(5-(2-aminopyridin-4-yl)-2-methyl-3H-imidazo[4,5-b]pyridin-3-yl)-2-fluorophenyl)piperazin-1-yl)ethoxy)ethoxy)ethoxy)ethyl)amino)-2-(2,6-dioxopiperidin-3-yl)isoindoline-1,3-dione (6) (11.3 mg, 13.5 μmol, 23% yield, 95% pure), a yellow semisolid. ¹H NMR (500 MHz, CDCl₃) δ 8.10-8.02 (m, 2H), 7.71 (d, J=8.2 Hz, 1H), 7.48 (dd, J=8.5, 7.1 Hz, 1H), 7.24-7.12 (m, 4H), 7.12-7.03 (m, 2H), 6.91 (d, J=8.5 Hz, 1H), 6.54 (t, J=5.6 Hz, 1H), 5.06 (s, 2H), 4.91-4.81 (m, 1H), 3.79 (q, J=5.1 Hz, 2H), 3.74 (t, J=5.2 Hz, 2H), 3.70-3.63 (m, 4H), 3.47 (q, J=5.5 Hz, 2H), 3.36-3.28 (m, 4H), 2.95 (bs, 4H), 2.88-2.83 (m, 2H), 2.83-2.66 (m, 3H), 2.57 (s, 3H), 2.14-2.03 (m, 1H). ¹⁹F NMR (470 MHz, CDCl₃) δ -119.45. LCMS (ESI) C₄₃H₄₇FN₁₀O₇ requires 834.91, found 418.80 (M/2+H).

### Example 7. Synthesis of 4-((14-(4-(4-(5-(2-aminopyridin-4-yl)-2-methyl-3H-imidazo[4,5-b]pyridin-3-yl)-2-fluorophenyl)piperazin-1-yl)-3,6,9,12-tetraoxatetradecyl)amino)-2-(2,6-dioxopiperidin-3-yl)isoindoline-1,3-dione, N,N-Diisopropylethylamine (7)

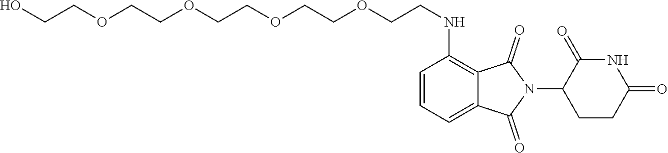 [SMILES: O=C1CCC(N2C(=O)c3cccc(NCCOCCOCCOCCOCCO)c3C2=O)C(=O)N1]

### 2-(2,6-dioxopiperidin-3-yl)-4-((14-hydroxy-3,6,9,12-tetraoxatetradecyl)amino)isoindoline-1,3-dione (7a)

To a 5 ml MWV equipped with a stir bar was added 14-amino-3,6,9,12-tetraoxatetradecan-1-ol (125 mg, 0.52 mmol, 1.0 eq.), DIPEA (0.55 mL, 3.16 mmol, 6.0 eq.), and 1,4-Dioxane (1.55 mL). This mixture was stirred for 5 minutes at room temperature under an argon atmosphere where 2-(2,6-dioxopiperidin-3-yl)-4-fluoroisoindoline-1,3-dione (C1) (175 mg, 0.63 mmol, 1.2 eq.) was added and the reaction mixture was stirred at 115° C. for 24 h. The reaction mixture was cooled to room temperature, concentrated under reduced pressure and purified via flash column chromatography using afford 2-(2,6-dioxopiperidin-3-yl)-4-((14-hydroxy-3,6,9,12-tetraoxatetradecyl)amino)isoindoline-1,3-dione (7a) (126 mg, 0.25 mmol, 48%), a green semisolid. ¹H NMR (400 MHz, CDCl₃) δ 8.47 (s, 1H), 7.49 (dd, J=8.5, 7.1 Hz, 1H), 7.11 (d, J=7.1 Hz, 1H), 6.92 (d, J=8.5 Hz, 1H), 4.91 (dd, J=12.0, 5.4 Hz, 1H), 3.78-3.71 (m, 4H), 3.71-3.65 (m, 12H), 3.65-3.58 (m, 3H), 3.47 (t, J=5.4 Hz, 2H), 2.93-2.66 (m, 3H), 2.18-2.08 (m, 1H). ¹³C NMR (101 MHz, CDCl₃) δ 171.37, 169.93, 168.60, 167.74, 146.97, 136.19, 132.69, 116.97, 111.84, 105.26, 77.48, 77.16, 76.84, 72.73, 71.00, 70.79, 70.73, 70.71, 70.62, 70.57, 69.53, 61.83, 49.00, 42.55, 31.55, 23.00.

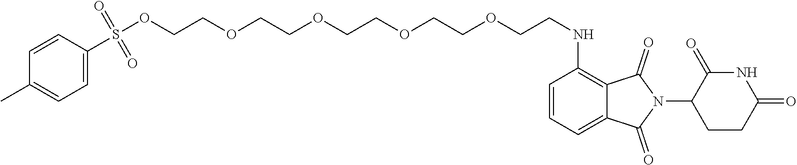 [SMILES: Cc1ccc(S(=O)(=O)OCCOCCOCCOCCOCCNc2cccc3c2C(=O)N(C2CCC(=O)NC2=O)C3=O)cc1]

### 14-((2-(2,6-dioxopiperidin-3-yl)-1,3-dioxoisoindolin-4-yl)amino)-3,6,9,12-tetraoxatetradecyl 4-methylbenzenesulfonate (7b)

In a 20 ml MWV equipped with a stir bar containing a solution of 2-(2,6-dioxopiperidin-3-yl)-4-((14-hydroxy-3,6,9,12-tetraoxatetradecyl)amino)isoindoline-1,3-dione (7a) (227 mg, 0.46 mmol, 1.0 eq.), triethylamine (0.19 mL, 1.38 mmol, 3.0 eq.) in DCM (9.00 mL) was added 4-toluenesulfonyl chloride (105 mg, 0.55 mmol, 1.2 eq.) at 0° C. The reaction mixture was allowed to warm up to room temperature and was stirred for 24 h. The reaction mixture was diluted with water and extracted with DCM (3×20 ml). The organic layers were recombined, dried over sodium sulfate, and concentrated under reduced pressure. The crude residue was purified by flash column chromatography using a DCM/MeOH gradient to give 14-((2-(2,6-dioxopiperidin-3-yl)-1,3-dioxoisoindolin-4-yl)amino)-3,6,9,12-tetraoxatetradecyl 4-methylbenzenesulfonate (7b) (165 mg, 0.25 mmol, 55% yield), a yellow semisolid. ¹³C NMR (101 MHz, CDCl₃) δ 171.22, 169.37, 168.48, 167.74, 146.95, 144.93, 136.17, 133.06, 132.62, 129.96, 128.11, 116.94, 111.77, 110.41, 70.90, 70.86, 70.73, 70.72, 70.71, 70.57, 69.58, 69.37, 68.80, 48.96, 42.51, 31.52, 22.92, 21.77.

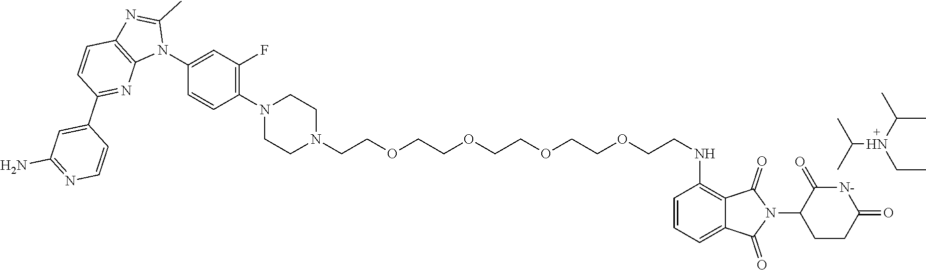

### 4-((14-(4-(4-(5-(2-aminopyridin-4-yl)-2-methyl-3H-imidazo[4,5-b]pyridin-3-yl)-2-fluorophenyl)piperazin-1-yl)-3,6,9,12-tetraoxatetradecyl)amino)-2-(2,6-dioxopiperidin-3-yl)isoindoline-1,3-dione, N,N-Diisopropylethylamine (7)

1,4-Dioxane (1.10 mL) and DIPEA (0.10 mL, 0.57 mmol, 10.0 eq.) were added sequentially to a dry 5 mL MWV under an atmosphere of argon containing 4-(3-(3-fluoro-4-(piperazin-1-yl)phenyl)-2-methyl-3H-imidazo[4,5-b]pyridin-5-yl)pyridin-2-amine (A7) (23 mg, 57 μmol, 1.0 eq.), 14-((2-(2,6-dioxopiperidin-3-yl)-1,3-dioxoisoindolin-4-yl)amino)-3,6,9,12-tetraoxatetradecyl 4-methylbenzenesulfonate (7b) (44 mg, 68 μmol, 1.2 eq.), and sodium iodide (2.1 mg, 14 μmol, 0.25 eq.). The reaction mixture was stirred at 90° C. for 16 hours where it was cooled down to room temperature and purified via flash column chromatography to give 4-((14-(4-(4-(5-(2-aminopyridin-4-yl)-2-methyl-3H-imidazo[4,5-b]pyridin-3-yl)-2-fluorophenyl)piperazin-1-yl)-3,6,9,12-tetraoxatetradecyl)amino)-2-(2,6-dioxopiperidin-3-yl)isoindoline-1,3-dione, N,N-Diisopropylethylamine (7) (25 mg, 57 μmol, 43% yield), a yellow semisolid. ¹H NMR (500 MHz, CDCl₃) δ 8.05 (t, J=7.3 Hz, 2H), 7.72 (d, J=8.1 Hz, 1H), 7.46 (t, J=8.1 Hz, 1H), 7.24-7.06 (m, 6H), 6.89 (d, J=8.5 Hz, 1H), 6.49 (t, J=5.6 Hz, 1H), 5.20 (s, 2H), 4.89 (dd, J=12.4, 5.2 Hz, 1H), 3.77-3.57 (m, 19H), 3.36-3.24 (m, 4H), 2.95-2.66 (m, 8H), 2.58 (s, 3H), 2.15-2.05 (m, 1H). Note, the product was isolated as a DIPEA salt and the NMR peaks that correlate to DIPEA are: ¹H NMR (500 MHz, CDCl₃) δ 11.13 (s, 1H), 3.44 (q, J=5.4 Hz, 2H), 3.09 (q, J=7.4 Hz, 2H), 1.67-1.31 (m, 15H). LCMS (ESI) C₄₅H₅₁FN₁₀Og requires 878.96, found 879 (M/Z+H).

### Example 8. Synthesis of 3-(2-(2-(2-(4-(4-(5-(2-aminopyridin-4-yl)-2-methyl-3H-imidazo[4,5-b]pyridin-3-yl)-2-fluorophenyl)piperazin-1-yl)ethoxy)ethoxy)ethoxy)-N-(1-(2-(2,6-dioxopiperidin-3-yl)-1,3-dioxoisoindolin-5-yl)azetidin-3-yl)propenamide (8)

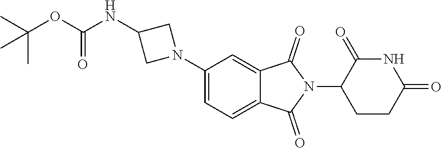 [SMILES: CC(C)(C)OC(=O)NC1CN(c2ccc3c(c2)C(=O)N(C2CCC(=O)NC2=O)C3=O)C1]

### tert-butyl (1-(2-(2,6-dioxopiperidin-3-yl)-1,3-dioxoisoindolin-5-yl)azetidin-3-yl)carbamate (8a)

To a 24 mL MWV equipped with a magnetic stir bar containing 2-(2,6-dioxopiperidin-3-yl)-5-fluoroisoindoline-1,3-dione (C1) (500 mg, 1.81 mmol, 1.0 eq.), tert-butyl azetidin-3-ylcarbamate (312 mg, 1.81 mmol, 1.0 eq.), and NMP (7.20 mL) was added DIPEA (0.78 mL, 4.53 mmol, 2.50 eq.). The reaction mixture was heated at 110° C. for 16 hours where it was then cooled to room temperature and quenched with DI water and extracted with ethyl acetate. The organic layer was washed with brine (4×) to remove NMP, dried over sodium sulfate, and concentrated under reduced pressure. The crude residue was purified by flash column chromatography using a CombiFlash to give tert-butyl (1-(2-(2,6-dioxopiperidin-3-yl)-1,3-dioxoisoindolin-5-yl)azetidin-3-yl)carbamate (8a) (170 mg, 397 μmol, 21.9%). ¹H NMR (500 MHz, CDCl₃) δ 8.17 (s, 1H), 7.64 (d, J=8.3 Hz, 1H), 6.78 (d, J=2.2 Hz, 1H), 6.53 (dd, J=8.3, 2.2 Hz, 1H), 5.12 (d, J=7.7 Hz, 1H), 4.93 (dd, J=12.2, 5.3 Hz, 1H), 4.68 (s, 1H), 4.34 (t, J=8.0 Hz, 2H), 3.83 (dd, J=8.5, 5.3 Hz, 2H), 2.92-2.67 (m, 3H), 2.19-2.08 (m, 1H), 1.46 (s, 9H). ¹³C NMR (126 MHz, DMSO) δ 173.69, 170.97, 168.30, 168.03, 155.44, 134.65, 125.70, 118.80, 115.76, 106.04, 105.16, 56.83, 55.95, 49.66, 41.36, 31.87, 23.07.

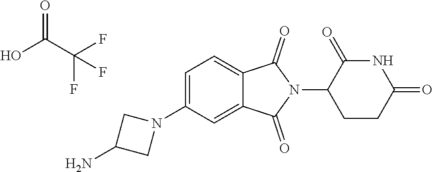 [SMILES: O=C(O)C(F)(F)F ; NC1CN(c2ccc3c(c2)C(=O)N(C2CCC(=O)NC2=O)C3=O)C1]

### 5-(3-aminoazetidin-1-yl)-2-(2,6-dioxopiperidin-3-yl)isoindoline-1,3-dione, Trifluoroacetic acid (8b)

To a 20 mL dram vial containing tert-butyl (1-(2-(2,6-dioxopiperidin-3-yl)-1,3-dioxoisoindolin-5-yl)azetidin-3-yl)carbamate (8a) (150 mg, 1 Eq, 350 μmol) in DCM (4.70 mL) was added trifluoroacetic acid (0.94 mL, 12.3 mmol, 35.0 eq.). The reaction mixture was stirred for 3 hours at room temperature where it was then concentrated under reduced pressure to give 5-(3-aminoazetidin-1-yl)-2-(2,6-dioxopiperidin-3-yl)isoindoline-1,3-dione, Trifluoroacetic acid (8b) and used in the next step without further purification. LCMS (ESI) C₁₆H₁₆N₄O₄ requires 328.33, found 329 (M/Z+H).

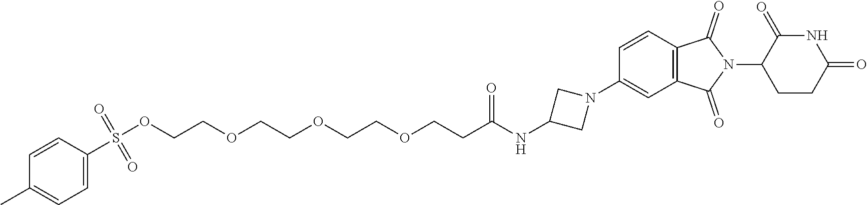 [SMILES: Cc1ccc(S(=O)(=O)OCCOCCOCCOCCC(=O)NC2CN(c3ccc4c(c3)C(=O)N(C3CCC(=O)NC3=O)C4=O)C2)cc1]

### 2-(2-(2-(3-((1-(2-(2,6-dioxopiperidin-3-yl)-1,3-dioxoisoindolin-5-yl)azetidin-3-yl)amino)-3-oxopropoxy)ethoxy)ethoxy)ethyl 4-methylbenzenesulfonate (8c)

To a 5 mL MWV equipped with a magnetic stir bar containing 5-(3-aminoazetidin-1-yl)-2-(2,6-dioxopiperidin-3-yl)isoindoline-1,3-dione, Trifluoroacetic acid (8b) (45 mg, 0.10 mmol, 1.0 eq.), 3-(2-(2-(2-(tosyloxy)ethoxy)ethoxy)ethoxy)propanoic acid, Trifluoroacetic acid (2c) (50 mg, 0.10 mmol, 1.0 eq.), and HATU (46 mg, 0.12 mmol, 1.0 eq.) was added DMF (1.00 mL) and DIPEA (0.11 mL, 0.61 mmol, 6.0 eq.) sequentially. The reaction mixture was stirred for 24 hours at room temperature where it was then concentrated under reduced pressure and purified via flash column chromatography to afford 2-(2-(2-(3-((1-(2-(2,6-dioxopiperidin-3-yl)-1,3-dioxoisoindolin-5-yl)azetidin-3-yl)amino)-3-oxopropoxy)ethoxy)ethoxy)ethyl 4-methylbenzenesulfonate (8c) (37 mg, 54 μmol, 53% yield, eluting at 5% MeOH in DCM), a yellow semisolid. LCMS (ESI) C₃₂H₃₈N₄O₁₁S requires 686.73, found 687.14 (M/Z+H).

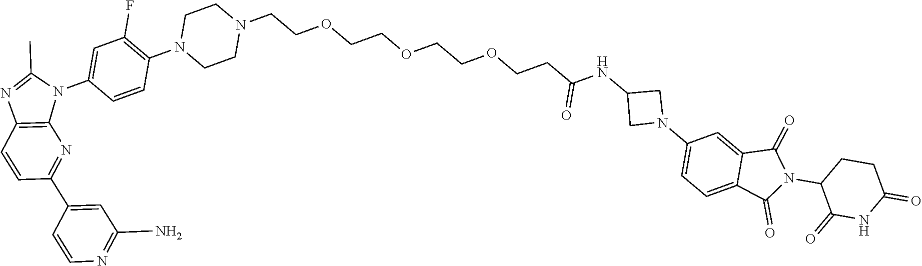 [SMILES: Cc1nc2ccc(-c3ccnc(N)c3)nc2n1-c1ccc(N2CCN(CCOCCOCCOCCC(=O)NC3CN(c4ccc5c(c4)C(=O)N(C4CCC(=O)NC4=O)C5=O)C3)CC2)c(F)c1]

### 3-(2-(2-(2-(4-(4-(5-(2-aminopyridin-4-yl)-2-methyl-3H-imidazo[4,5-b]pyridin-3-yl)-2-fluorophenyl)piperazin-1-yl)ethoxy)ethoxy)ethoxy)-N-(1-(2-(2,6-dioxopiperidin-3-yl)-1,3-dioxoisoindolin-5-yl)azetidin-3-yl)propenamide (8)

1,4-dioxane (1.00 mL) and DIPEA (28 mg, 0.22 mmol, 10.0 eq.) were added sequentially to a 5 mL MWV equipped with a magnetic stir bar containing 4-(3-(3-fluoro-4-(piperazin-1-yl)phenyl)-2-methyl-3H-imidazo[4,5-b]pyridin-5-yl)pyridin-2-amine (A7) (10 mg, 24 μmol, 1.1 eq.), sodium iodide (1.3 mg, 8.7 μmol, 0.4 eq.), and 2-(2-(2-(3-((1-(2-(2,6-dioxopiperidin-3-yl)-1,3-dioxoisoindolin-5-yl)azetidin-3-yl)amino)-3-oxopropoxy)ethoxy)ethoxy)ethyl 4-methylbenzenesulfonate (8c) (15 mg, 22 μmol, 1.0 eq.). The reaction mixture was stirred at 90° C. for 16 hours where the reaction mixture was allowed to cool to room temperature and was concentrated under reduced pressure. The crude residue was purified by flash column chromatography using a DCM/MeOH gradient to give title compound 3-(2-(2-(2-(4-(4-(5-(2-aminopyridin-4-yl)-2-methyl-3H-imidazo[4,5-b]pyridin-3-yl)-2-fluorophenyl)piperazin-1-yl)ethoxy)ethoxy)ethoxy)-N-(1-(2-(2,6-dioxopiperidin-3-yl)-1,3-dioxoisoindolin-5-yl)azetidin-3-yl)propanamide (8) (9 mg, 0.01 mmol, 40% yield), a yellow semisolid. LCMS (ESI) C₄₇H₅₂FN₁₁O₈ requires 918.00, found 460.13 (M/2+H). ¹H NMR (500 MHz, CDCl₃) δ 8.03 (dd, J=8.2, 4.2 Hz, 1H), 7.80-7.62 (m, 5H), 7.55-7.36 (m, 1H), 7.27-7.06 (m, 7H), 4.92-4.80 (m, 1H), 4.79-4.63 (m, 1H), 4.21 (dt, J=7.9, 3.8 Hz, 1H), 3.94-3.77 (m, 3H), 3.70 (dt, J=6.0, 3.0 Hz, 2H), 3.66-3.52 (m, 8H), 3.53-3.43 (m, 3H), 3.43-3.26 (m, 2H), 3.25-3.11 (m, 3H), 3.05 (dtt, J=10.6, 5.8, 2.5 Hz, 4H), 2.81-2.73 (m, 1H), 2.74-2.63 (m, 1H), 2.60-2.53 (m, 3H), 2.51 (td, J=6.0, 2.2 Hz, 1H), 2.29 (s, 4H). ¹⁹F NMR (471 MHz, CDCl₃) δ -119.60. LCMS (ESI) C₄₇H₅₂FN₁₁O₈ requires 918.00, found 460.13 (M/2+H).

### Example 9. Synthesis of 5-(4-(3-(2-(2-(2-(4-(4-(5-(2-aminopyridin-4-yl)-2-methyl-3H-imidazo[4,5-b]pyridin-3-yl)-2-fluorophenyl)piperazin-1-yl)ethoxy)ethoxy)ethoxy)propanoyl)piperazin-1-yl)-2-(2,6-dioxopiperidin-3-yl)isoindoline-1,3-dione (9)

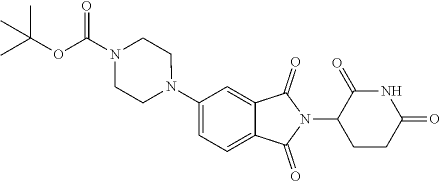 [SMILES: CC(C)(C)OC(=O)N1CCN(c2ccc3c(c2)C(=O)N(C2CCC(=O)NC2=O)C3=O)CC1]

### tert-butyl 4-(2-(2,6-dioxopiperidin-3-yl)-1,3-dioxoisoindolin-5-yl)piperazine-1-carboxylate (9a)

To a 24 mL MWV equipped with a magnetic stir bar containing 2-(2,6-dioxopiperidin-3-yl)-5-fluoroisoindoline-1,3-dione (C1) (500 mg, 1.81 mmol, 1.0 eq.), tert-butyl piperazine-1-carboxylate (337 mg, 1.81 mmol, 1.0 eq.), and NMP (7.20 mL) was added DIPEA (0.78 mL 4.53 mmol, 2.5 eq.). The reaction mixture was heated at 110° C. for 16 hours where it was cooled down to room temperature and was quenched with DI water and extracted with ethyl acetate. The organic layer was washed with brine (4×) to remove NMP, dried over sodium sulfate, and concentrated under reduced pressure. The crude residue was purified via flash column chromatography to give tert-butyl 4-(2-(2,6-dioxopiperidin-3-yl)-1,3-dioxoisoindolin-5-yl)piperazine-1-carboxylate (9a) (510 mg, 1.15 mmol, 63%), a yellow solid. LCMS (ESI) C₂₂H₂₆N₄O₆ requires 442.47, found 387.93 (M/Z+H, tert-butyl cleaved in LCMS so the free carbamic acid was measured).

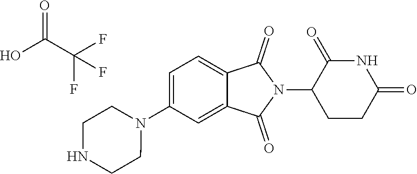

2-(2,6-dioxopiperidin-3-yl)-5-(piperazin-1-yl)isoindoline-1,3-dione, Trifluoroacetic acid (9b) To a 20 mL dram vial containing To a 20 mL dram vial containing tert-butyl 4-(2-(2,6-dioxopiperidin-3-yl)-1,3-dioxoisoindolin-5-yl)piperazine-1-carboxylate (9a) (510 mg, 1.15 mmol, 1.0 eq.) in DCM (12.4 mL) was added TFA (3.11 mL, 40.3 mmol, 3.5 eq.). The reaction mixture was stirred for 3 hours at room temperature where it was then concentrated under reduced pressure to give 2-(2,6-dioxopiperidin-3-yl)-5-(piperazin-1-yl)isoindoline-1,3-dione, Trifluoroacetic acid (9b) in quantitative yield as a yellow solid. This was used in the next step without further purification. ¹H NMR (500 MHz, DMSO) δ 11.09 (s, 1H), 8.99 (s, 2H), 7.75 (d, J=8.5 Hz, 1H), 7.45 (d, J=2.3 Hz, 1H), 7.33 (dd, J=8.6, 2.4 Hz, 1H), 5.09 (dd, J=12.8, 5.4 Hz, 1H), 3.67 (t, J=5.3 Hz, 4H), 3.25 (d, J=6.1 Hz, 4H), 2.89 (ddd, J=16.6, 13.7, 5.4 Hz, 1H), 2.64-2.51 (m, 2H), 2.08-1.98 (m, 1H). ¹³C NMR (126 MHz, DMSO) δ 172.79, 170.02, 167.40, 166.90, 154.43, 133.80, 124.99, 119.75, 118.66, 108.89, 48.85, 44.26, 42.30, 40.02, 39.95, 39.85, 39.78, 39.69, 39.61, 39.52, 39.44, 39.35, 39.19, 39.02, 30.97, 22.14.

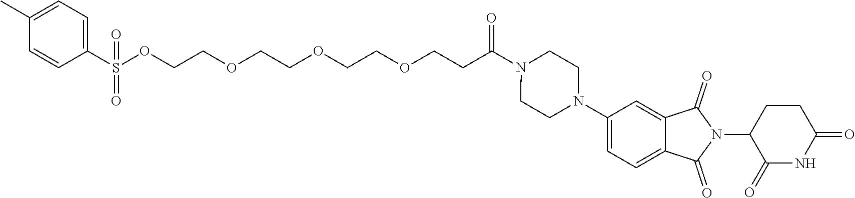 [SMILES: Cc1ccc(S(=O)(=O)OCCOCCOCCOCCC(=O)N2CCN(c3ccc4c(c3)C(=O)N(C3CCC(=O)NC3=O)C4=O)CC2)cc1]

### 2-(2-(2-(3-(4-(2-(2,6-dioxopiperidin-3-yl)-1,3-dioxoisoindolin-5-yl)piperazin-1-yl)-3-oxopropoxy)ethoxy)ethoxy)ethyl 4-methylbenzenesulfonate (9c)

To a 5 mL MWV equipped with a magnetic stir bar containing 2-(2,6-dioxopiperidin-3-yl)-5-(piperazin-1-yl)isoindoline-1,3-dione, Trifluoroacetic acid (9b) (45 mg, 0.1 mmol, 1.0 eq.), 3-(2-(2-(2-(tosyloxy)ethoxy)ethoxy)ethoxy)propanoic acid, Trifluoroacetic acid (2c) (48 mg, 0.1 mmol, 1.0 eq.), and HATU (45 mg, 1.2 Eq, 0.12 mmol) was added DMF (1.00 mL) and DIPEA (0.10 mL, 0.59 mmol, 6.0 eq.) sequentially. The reaction mixture was stirred at room temperature for 24 hours where it was then concentrated under reduced pressure and purified by flash column chromatography to give 2-(2-(2-(3-(4-(2-(2,6-dioxopiperidin-3-yl)-1,3-dioxoisoindolin-5-yl)piperazin-1-yl)-3-oxopropoxy)ethoxy)ethoxy)ethyl 4-methylbenzenesulfonate (9c) (56 mg, 70 μmol, 71% yield, 88% purity), a yellow semisolid. ¹H NMR (500 MHz, CDCl₃) δ 8.24-8.09 (m, 2H), 7.76 (d, J=8.1 Hz, 2H), 7.72 (d, J=8.4 Hz, 1H), 7.37-7.30 (m, 2H), 7.19-7.08 (m, 1H), 4.94 (dd, J=12.3, 5.3 Hz, 1H), 4.15-4.10 (m, 2H), 3.91-3.77 (m, 5H), 3.70-3.52 (m, 10H), 3.54-3.43 (m, 3H), 3.11 (qd, J=7.4, 4.1 Hz, 2H), 2.99-2.95 (m, 1H), 2.91-2.79 (m, 2H), 2.78-2.70 (m, 2H), 2.43 (s, 3H), 2.18-2.10 (m, 1H). ¹³C NMR (126 MHz, CDCl₃) δ 171.09, 168.33, 167.67, 167.14, 163.40, 145.06, 134.37, 132.95, 130.01, 128.07, 125.62, 118.95, 109.37, 77.42, 77.16, 76.91, 70.83, 70.60, 70.52, 69.44, 68.79, 67.55, 54.91, 49.35, 45.32, 43.07, 41.40, 37.32, 33.51, 31.55, 22.81, 21.77. LCMS (ESI) C₃₃H₄₀N₄O₁₁S requires 700.76, found 701.97.

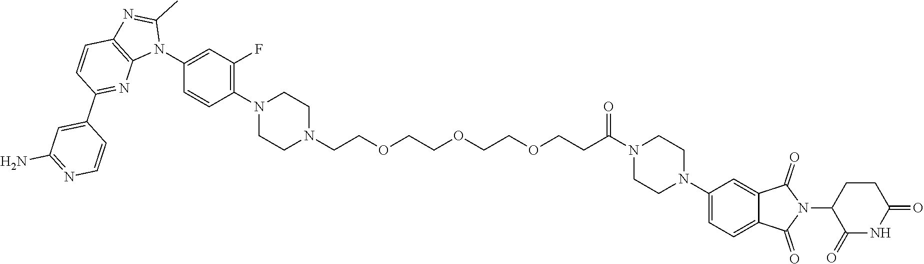 [SMILES: Cc1nc2ccc(-c3ccnc(N)c3)nc2n1-c1ccc(N2CCN(CCOCCOCCOCCC(=O)N3CCN(c4ccc5c(c4)C(=O)N(C4CCC(=O)NC4=O)C5=O)CC3)CC2)c(F)c1]

### 5-(4-(3-(2-(2-(2-(4-(4-(5-(2-aminopyridin-4-yl)-2-methyl-3H-imidazo[4,5-b]pyridin-3-yl)-2-fluorophenyl)piperazin-1-yl)ethoxy)ethoxy)ethoxy)propanoyl)piperazin-1-yl)-2-(2,6-dioxopiperidin-3-yl)isoindoline-1,3-dione (9)

1,4-dioxane (1.00 mL) and DIPEA (30 mg, 0.23 mmol, 10.0 eq.) were added sequentially to a 5 mL MWV equipped with a magnetic stir bar containing 4-(3-(3-fluoro-4-(piperazin-1-yl)phenyl)-2-methyl-3H-imidazo[4,5-b]pyridin-5-yl)pyridin-2-amine (A7) (10 mg, 25 μmol, 1.1 eq.), sodium iodide (1.4 mg, 9.1 μmol, 0.4 eq.), and 2-(2-(2-(3-(4-(2-(2,6-dioxopiperidin-3-yl)-1,3-dioxoisoindolin-5-yl)piperazin-1-yl)-3-oxopropoxy)ethoxy)ethoxy)ethyl 4-methylbenzenesulfonate (9c) (16 mg, 23 μmol, 1.0 eq.). The reaction mixture was stirred at 90° C. for 16 hours where the reaction mixture was allowed to cool to room temperature and was concentrated under reduced pressure. The crude residue was purified by flash column chromatography to give title compound 5-(4-(3-(2-(2-(2-(4-(4-(5-(2-aminopyridin-4-yl)-2-methyl-3H-imidazo[4,5-b]pyridin-3-yl)-2-fluorophenyl)piperazin-1-yl)ethoxy)ethoxy)ethoxy)propanoyl)piperazin-1-yl)-2-(2,6-dioxopiperidin-3-yl)isoindoline-1,3-dione (9) (7.3 mg, 7.8 μmol, 34% yield, 94% purity), a yellow semisolid. ¹H NMR (500 MHz, CDCl₃) δ 8.03 (dd, J=8.5, 3.2 Hz, 1H), 7.81-7.75 (m, 1H), 7.70-7.63 (m, 4H), 7.62-7.54 (m, 1H), 7.44 (t, J=1.8 Hz, 1H), 7.20-7.14 (m, 2H), 7.15-7.08 (m, 4H), 6.99-6.95 (m, 1H), 4.88-4.81 (m, 1H), 3.92-3.83 (m, 2H), 3.80-3.61 (m, 7H), 3.62-3.54 (m, 3H), 3.38 (t, J=5.2 Hz, 2H), 3.34-3.28 (m, 3H), 3.29-3.25 (m, 4H), 3.07-2.98 (m, 4H), 2.79-2.72 (m, 1H), 2.72-2.63 (m, 1H), 2.63-2.57 (m, 2H), 2.53 (s, 3H), 2.27 (s, 5H), 2.08-1.99 (m, 1H). ¹⁹F NMR (471 MHz, CDCl₃) δ -119.76. LCMS (ESI) C₄₈H₅₄FN₁₁O₈ requires 932.03, found 467.25 (M/2+H).

### Example 10. Synthesis of 3-(4-((4-((4-(3-(2-(2-(4-(4-(5-(2-aminopyridin-4-yl)-2-methyl-3H-imidazo[4,5-b]pyridin-3-yl)-2-fluorophenyl)piperazin-1-yl)ethoxy)ethoxy)propanoyl)piperazin-1-yl)methyl)benzyl)amino)-1-oxoisoindolin-2-yl)piperidine-2,6-dione (10)

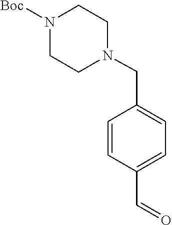 [SMILES: CC(C)(C)OC(=O)N1CCN(Cc2ccc(C=O)cc2)CC1]

### tert-butyl 4-(4-formylbenzyl)piperazine-1-carboxylate (10a)

triethylamine (0.46 mL, 3.32 mmol, 1.1 eq.) was added to a 8 mL MWV containing 4-(bromomethyl)benzaldehyde (600 mg, 3.01 mmol, 1.0 eq.), tert-butyl piperazine-1-carboxylate (674 mg, 3.62 mmol, 1.2 eq.), and MeCN (6 mL). The reaction mixture was stirred at room temperature for 16 hours where it was then concentrated under reduced pressure and purified by flash column chromatography to give tert-butyl 4-(4-formylbenzyl)piperazine-1-carboxylate (10a) (729 mg, 2.39 mmol, 79% yield, eluting at 17% ethyl acetate in hexanes), a yellow oil. ¹H NMR (500 MHz, CDCl₃) δ 10.00 (s, 1H), 7.84 (d, J=8.2 Hz, 2H), 7.51 (d, J=7.9 Hz, 2H), 3.58 (s, 2H), 3.44 (t, J=5.0 Hz, 4H), 2.40 (t, J=5.0 Hz, 4H), 1.45 (s, 9H). ¹³C NMR (126 MHz, CDCl₃) δ 192.08, 154.91, 145.56, 135.75, 129.97, 129.59, 79.81, 62.80, 53.12, 28.56.

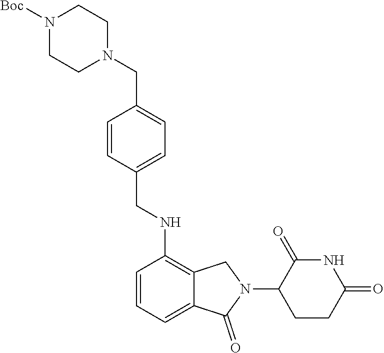 [SMILES: CC(C)(C)OC(=O)N1CCN(Cc2ccc(CNc3cccc4c3CN(C3CCC(=O)NC3=O)C4=O)cc2)CC1]

### tert-butyl 4-(4-(((2-(2,6-dioxopiperidin-3-yl)-1-oxoisoindolin-4-yl)amino)methyl)benzyl)piperazine-1-carboxylate (10b)

Acetic acid (69 mg, 1.15 mmol, 1.0 eq.) was added to a 8 mL MWV containing tert-butyl 4-(4-formylbenzyl)piperazine-1-carboxylate (10a) (350 mg, 1.15 mmol, 1.0 eq.), 3-(4-amino-1-oxoisoindolin-2-yl)piperidine-2,6-dione (298 mg, 1.15 mmol, 1.0 eq.), and DCM (4.72 mL). The reaction mixture was stirred for 30 minutes at 30° C. where sodium triacetoxyborohydride (244 mg, 1.15 mol, 1.0 eq.) was added and the reaction mixture was stirred at room temperature for days with no sign of reaction progression. The reaction was then run at 80 C and after 6 hours, there was no starting material left by TLC. Saturated sodium bicarbonate was added, and the reaction mixture was extracted with DCM (3×15 mL). The organic layers were recombined, dried over sodium sulfate, and concentrated under reduced pressure. The residue was purified by automated flash column chromatography using a CombiFlash to give tert-butyl 4-(4-(((2-(2,6-dioxopiperidin-3-yl)-1-oxoisoindolin-4-yl)amino)methyl)benzyl)piperazine-1-carboxylate (10b) (371 mg, 0.67 mmol, 58% yield, eluting at 5% MeOH in DCM), a white solid. ¹H NMR (500 MHz, CDCl₃) δ 8.56 (s, 1H), 7.35-7.27 (m, 7H), 7.26-7.24 (m, 1H), 6.78 (dd, J=8.0, 0.9 Hz, 1H), 5.17 (dd, J=13.2, 5.2 Hz, 1H), 4.40 (s, 2H), 4.30 (d, J=15.6 Hz, 1H), 4.13 (d, J=15.6 Hz, 1H), 3.42 (t, J=5.1 Hz, 4H), 2.87-2.67 (m, 2H), 2.38 (t, J=5.1 Hz, 4H), 2.22 (qd, J=13.0, 5.1 Hz, 1H), 2.15-2.08 (m, 1H), 1.44 (s, 9H). ¹³C NMR (126 MHz, CDCl₃) δ 171.53, 170.11, 170.04, 154.91, 143.01, 137.60, 137.31, 131.97, 129.89, 129.74, 129.53, 127.62, 127.12, 126.50, 113.53, 113.13, 79.76, 65.18, 62.74, 52.92, 51.87, 47.85, 45.16, 31.64, 28.55, 23.54. HCl

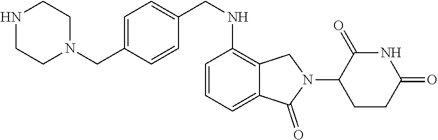 [SMILES: O=C1CCC(N2Cc3c(NCc4ccc(CN5CCNCC5)cc4)cccc3C2=O)C(=O)N1]

### 3-(1-oxo-4-((4-(piperazin-1-ylmethyl)benzyl)amino)isoindolin-2-yl)piperidine-2,6-dione, HCl (10c)

HCl (2.5 mL, 10.0 mmol, 18.0 eq., 4M in 1,4-dioxane) w as added to a 20 mL dram vial containing tert-butyl 4-(4-(((2-(2,6-dioxopiperidin-3-yl)-1-oxoisoindolin-4-yl)amino)methyl)benzyl)piperazine-1-carboxylate (10b) (302 mg, 0.55 mmol, 1.0 eq.). The reaction mixture was stirred for 2 hours where it was then concentrated under reduced pressure to give 3-(1-oxo-4-((4-(piperazin-1-ylmethyl)benzyl)amino)isoindolin-2-yl)piperidine-2,6-dione, HCl (10c) (225 mg, 0.55 mmol, 84% yield), a light-brown solid. This was used without further purification. LCMS (ESI) C₂₅H₂₉N₅O₃ requires 447.22, found 448.10.

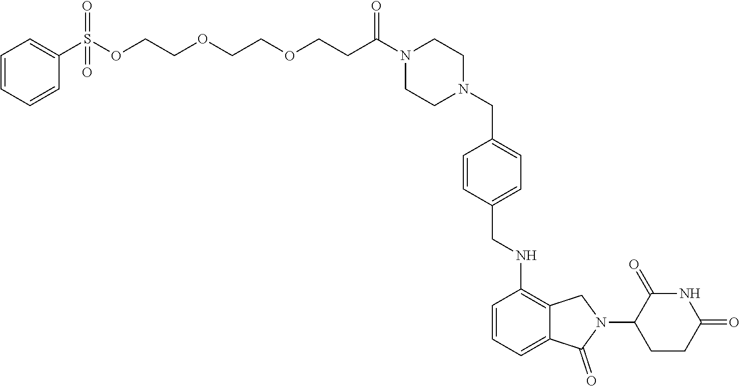 [SMILES: O=C1CCC(N2Cc3c(NCc4ccc(CN5CCN(C(=O)CCOCCOCCOS(=O)(=O)c6ccccc6)CC5)cc4)cccc3C2=O)C(=O)N1]

### 2-(2-(3-(4-(4-(((2-(2,6-dioxopiperidin-3-yl)-1-oxoisoindolin-4-yl)amino)methyl)benzyl)piperazin-1-yl)-3-oxopropoxy)ethoxy)ethyl benzenesulfonate (10d)

DMF (1.0 mL) and DIPEA (33 mg, 0.26 mmol, 5.0 eq.) were added to a 5 mL MWV equipped with a magnetic stir bar containing HATU (24 mg, 0.06 mmol, 1.2 eq.), 3-(1-oxo-4-((4-(piperazin-1-ylmethyl)benzyl)amino)isoindolin-2-yl)piperidine-2,6-dione, HCl (10c) (25 mg, 52 μmol, 1.0 eq.), and 3-(2-(2-((phenylsulfonyl)oxy)ethoxy)ethoxy)propanoic acid (16 mg, 52 μmol, 1.0 eq.). The reaction mixture was stirred at room temperature for 16 hours where it was then diluted with water and extracted with DCM. The organic layers was washed with a saturated solution of sodium bicarbonate and brine. The organic layer was dried over sodium sulfate, concentrated under reduced pressure, and purified by flash column chromatography to give 2-(2-(3-(4-(4-(((2-(2,6-dioxopiperidin-3-yl)-1-oxoisoindolin-4-yl)amino)methyl)benzyl)piperazin-1-yl)-3-oxopropoxy)ethoxy)ethyl benzenesulfonate (10d) (18 mg, 24 μmol, 47% yield). LCMS (ESI) C₃₈H₄₅N₅O₉S requires 747.86, found 748.29 (M/Z+H).

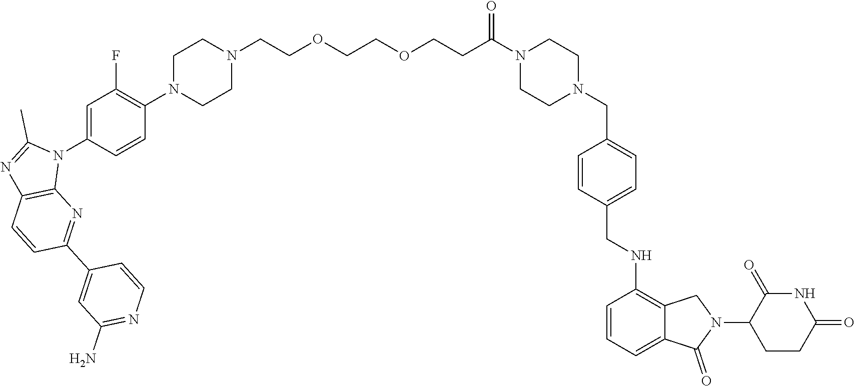 [SMILES: Cc1nc2ccc(-c3ccnc(N)c3)nc2n1-c1ccc(N2CCN(CCOCCOCCC(=O)N3CCN(Cc4ccc(CNc5cccc6c5CN(C5CCC(=O)NC5=O)C6=O)cc4)CC3)CC2)c(F)c1]

### 3-(4-((4-((4-(3-(2-(2-(4-(4-(5-(2-aminopyridin-4-yl)-2-methyl-3H-imidazo[4,5-b]pyridin-3-yl)-2-fluorophenyl)piperazin-1-yl)ethoxy)ethoxy)propanoyl)piperazin-1-yl)methyl)benzyl)amino)-1-oxoisoindolin-2-yl)piperidine-2,6-dione (10)

1,4-Dioxane (1.00 mL) and DIPEA (31 mg, 0.24 mmol, 10.0 eq.) were added to an 5 mL MWV containing 4-(3-(3-fluoro-4-(piperazin-1-yl)phenyl)-2-methyl-3H-imidazo[4,5-b]pyridin-5-yl)pyridin-2-amine (A7) (11 mg, 26 μmol, 1.1 eq.), 2-(2-(3-(4-(4-(((2-(2,6-dioxopiperidin-3-yl)-1-oxoisoindolin-4-yl)amino)methyl)benzyl)piperazin-1-yl)-3-oxopropoxy)ethoxy)ethyl benzenesulfonate (10d) (18 mg, 24 μmol, 1.0 eq.), and sodium iodide (1.4 mg, 9.6 μmol, 0.4 eq.). The reaction mixture was stirred at 90° C. for 16 hours where it was then concentrated under reduced pressure and purified via flash column chromatography to give 3-(4-((4-((4-(3-(2-(2-(4-(4-(5-(2-aminopyridin-4-yl)-2-methyl-3H-imidazo[4,5-b]pyridin-3-yl)-2-fluorophenyl)piperazin-1-yl)ethoxy)ethoxy)propanoyl)piperazin-1-yl)methyl)benzyl)amino)-1-oxoisoindolin-2-yl)piperidine-2,6-dione (10) (10 mg, 10 μmol, 42% yield), a white solid. LCMS (ESI) C₅₄H₆₁FN₁₂₀₆ requires 993.16, found 994 (M/Z+H).

### Example 11. Synthesis of 3-(4-((4-((4-(3-(4-(4-(5-(2-aminopyridin-4-yl)-2-methyl-3H-imidazo[4,5-b]pyridin-3-yl)-2-fluorophenyl)piperazin-1-yl)-3-oxopropyl)piperazin-1-yl)methyl)benzyl)amino)-1-oxoisoindolin-2-yl)piperidine-2,6-dione (11)

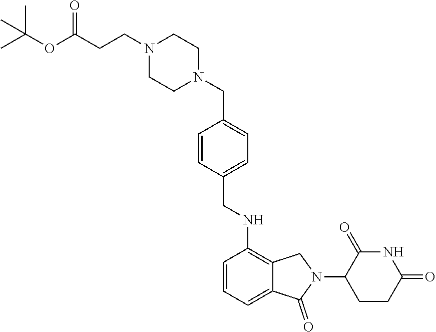 [SMILES: CC(C)(C)OC(=O)CCN1CCN(Cc2ccc(CNc3cccc4c3CN(C3CCC(=O)NC3=O)C4=O)cc2)CC1]

### tert-butyl 3-(4-(4-(((2-(2,6-dioxopiperidin-3-yl)-1-oxoisoindolin-4-yl)amino)methyl)benzyl)piperazin-1-yl)propanoate (11a)

To a 8 mL MWV equipped with a magnetic stir bar containing 3-(1-oxo-4-((4-(piperazin-1-ylmethyl)benzyl)amino)isoindolin-2-yl)piperidine-2,6-dione (10c), HCl (30 mg, 1 Eq, 62 μmol) in MeCN (1.00 mL) was added tert-butyl 3-bromopropanoate (14 mg, 1.05 Eq, 65 μmol), KI (2.1 mg, 0.2 Eq, 12 μmol), and DIPEA (12 mg, 16 μL, 1.5 Eq, 93 μmol). The reaction mixture was stirred for 16 hours at 80° C. After completion of the reaction, the reaction mixture was concentrated under reduced pressure and purified by automated column chromatography to give tert-butyl 3-(4-(4-(((2-(2,6-dioxopiperidin-3-yl)-1-oxoisoindolin-4-yl)amino)methyl)benzyl)piperazin-1-yl)propanoate (11a) (16 mg, 28 μmol, 45%). LCMS (ESI) C₃₂H₄₁N₅O₅ requires 575.71, found 576 (M/Z+H).

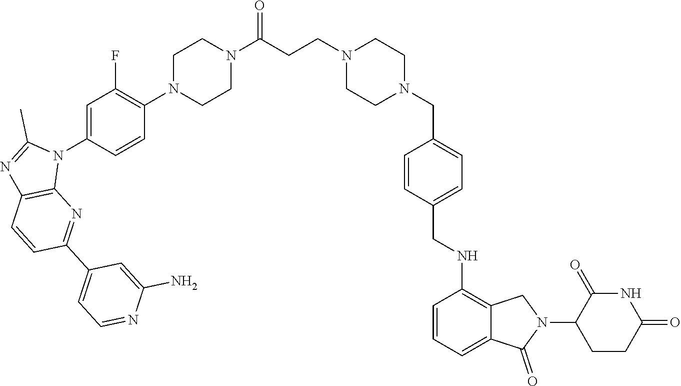 [SMILES: Cc1nc2ccc(-c3ccnc(N)c3)nc2n1-c1ccc(N2CCN(C(=O)CCN3CCN(Cc4ccc(CNc5cccc6c5CN(C5CCC(=O)NC5=O)C6=O)cc4)CC3)CC2)c(F)c1]

### 3-(4-((4-((4-(3-(4-(4-(5-(2-aminopyridin-4-yl)-2-methyl-3H-imidazo[4,5-b]pyridin-3-yl)-2-fluorophenyl)piperazin-1-yl)-3-oxopropyl)piperazin-1-yl)methyl)benzyl)amino)-1-oxoisoindolin-2-yl)piperidine-2,6-dione (11)

To a 8 mL MWV equipped with a magnetic stir bar containing tert-butyl 3-(4-(4-(((2-(2,6-dioxopiperidin-3-yl)-1-oxoisoindolin-4-yl)amino)methyl)benzyl)piperazin-1-yl)propanoate (11a) (16 mg, 28 μmol, 1.0 eq.) in DCM (0.50 mL) was added TFA (0.11 mL) and was stirred at room temperature for 16 hours where it was then concentrated under reduced pressure to give 3-(4-(4-(((2-(2,6-dioxopiperidin-3-yl)-1-oxoisoindolin-4-yl)amino)methyl)benzyl)piperazin-1-yl)propanoic acid, Trifluoroacetic acid (11b) in quantitative yield and was used directly in the next step without further purification. To a 8 ml MWV equipped with a magnetic stir bar containing 3-(4-(4-(((2-(2,6-dioxopiperidin-3-yl)-1-oxoisoindolin-4-yl)amino)methyl)benzyl)piperazin-1-yl)propanoic acid, Trifluoroacetic acid (11b) (18 mg, 28 μmol, 1.0 eq.), 4-(3-(3-fluoro-4-(piperazin-1-yl)phenyl)-2-methyl-3H-imidazo[4,5-b]pyridin-5-yl)pyridin-2-amine (A7) (11 mg, 27 μmol, 1.0 eq.), and HATU (12 mg, 32 μmol, 1.2 eq.) was added DMF (0.50 mL) and DIPEA (22 mg, 0.17 mmol, 6.2 eq.). The reaction mixture was stirred at room temperature for 16 hours where it was concentrated under reduced pressure and purified by flash column chromatography to give 3-(4-((4-((4-(3-(4-(4-(5-(2-aminopyridin-4-yl)-2-methyl-3H-imidazo[4,5-b]pyridin-3-yl)-2-fluorophenyl)piperazin-1-yl)-3-oxopropyl)piperazin-1-yl)methyl)benzyl)amino)-1-oxoisoindolin-2-yl)piperidine-2,6-dione (11) (17 mg, 19 μmol, 69%, eluting at 30% MeOH in DCM). ¹H NMR (500 MHz, DMSO) δ 8.21 (dd, J=4.2, 1.5 Hz, 1H), 8.11 (d, J=8.3 Hz, 1H), 8.01-7.89 (m, 2H), 7.81 (d, J=8.3 Hz, 1H), 7.57 (dd, J=13.1, 2.4 Hz, 1H), 7.41 (dd, J=8.5, 2.4 Hz, 1H), 7.36-7.30 (m, 1H), 7.30-7.14 (m, 6H), 7.08-7.01 (m, 2H), 7.00 (s, 1H), 5.98 (s, 2H), 5.12 (t, J=5.8 Hz, 1H), 4.51-4.39 (m, 2H), 4.40-4.31 (m, 2H), 3.66 (1, J=5.8 Hz, 5H), 3.41 (d, J=7.3 Hz, 2H), 3.18-3.07 (m, 5H). LCMS (ESI) C₅₀H₅₃FN₁₂₀₄ requires 905.05, found 906 (M/Z+H).

### Example 12. Synthesis of 3-(4-((4-((4-(4-(4-(4-(5-(2-aminopyridin-4-yl)-2-methyl-3H-imidazo[4,5-b]pyridin-3-yl)-2-fluorophenyl)piperazin-1-yl)-4-oxobutyl)piperazin-1-yl)methyl)benzyl)amino)-1-oxoisoindolin-2-yl)piperidine-2,6-dione (12)

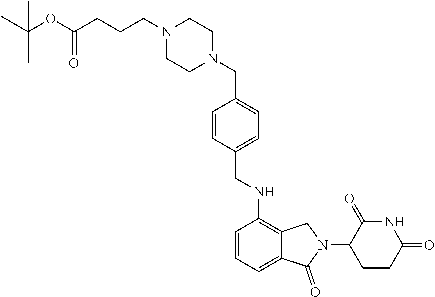 [SMILES: CC(C)(C)OC(=O)CCCN1CCN(Cc2ccc(CNc3cccc4c3CN(C3CCC(=O)NC3=O)C4=O)cc2)CC1]

### tert-butyl 4-(4-(4-(((2-(2,6-dioxopiperidin-3-yl)-1-oxoisoindolin-4-yl)amino)methyl)benzyl)piperazin-1-yl) butanoate (12a)

DMF (1.00 mL), N-ethyl-N-isopropylpropan-2-amine (26 mg, 0.20 mmol, 3.0 eq.), and tert-butyl 4-bromobutanoate (18 mg, 80 μmol, 1.2 eq.) were added sequentially to a 8 mL MWV equipped with a stir bar containing 3-(1-oxo-4-((4-(piperazin-1-ylmethyl)benzyl)amino)isoindolin-2-yl)piperidine-2,6-dione (10c) (30 mg, 67 μmol, 1.0 eq.) and potassium iodide (11 mg, 67 μmol, 1.0 eq.). The reaction mixture was stirred at 80° C. for 24 hours. It was allowed to cool to ambient temperature and was concentrated under reduced pressure. The crude residue was purified by flash column chromatography to give tert-butyl 4-(4-(4-(((2-(2,6-dioxopiperidin-3-yl)-1-oxoisoindolin-4-yl)amino)methyl)benzyl)piperazin-1-yl) butanoate (12a) (31 mg, 53 μmol, 78%), a clear semisolid. LCMS (ESI) C₃₃H₄₃N₅O₅ requires 589.32, found 590.36.

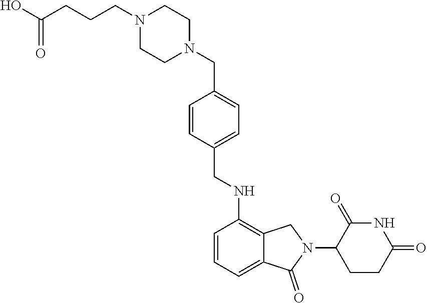 [SMILES: O=C(O)CCCN1CCN(Cc2ccc(CNc3cccc4c3CN(C3CCC(=O)NC3=O)C4=O)cc2)CC1]

4-(4-(4-(((2-(2,6-dioxopiperidin-3-yl)-1-oxoisoindolin-4-yl)amino)methyl)benzyl)piperazin-1-yl) butanoic acid, Trifluoroacetic acid (12b)

To a 20 mL dram vial containing tert-butyl 4-(4-(4-(((2-(2,6-dioxopiperidin-3-yl)-1-oxoisoindolin-4-yl)amino)methyl)benzyl)piperazin-1-yl) butanoate (12a) (31 mg, 53 μmol, 1.0 eq.) in DCM (0.48 mL) was added trifluoroacetic acid (0.12 mL, 1.6 mmol, 30.0 eq.). The reaction mixture was concentrated under reduced pressure to give 4-(4-(4-(((2-(2,6-dioxopiperidin-3-yl)-1-oxoisoindolin-4-yl)amino)methyl)benzyl)piperazin-1-yl) butanoic acid, Trifluoroacetic acid in quantitative yield and was used in the next step without further purification. LCMS (ESI) C₂₉H₃₅N₅O₅ requires 533.63, found 534.24 (M/Z+H).

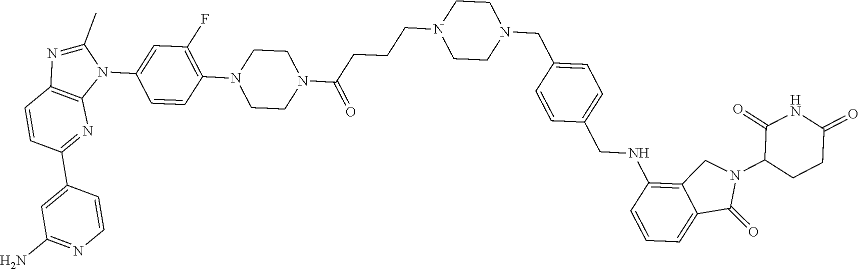 [SMILES: Cc1nc2ccc(-c3ccnc(N)c3)nc2n1-c1ccc(N2CCN(C(=O)CCCN3CCN(Cc4ccc(CNc5cccc6c5CN(C5CCC(=O)NC5=O)C6=O)cc4)CC3)CC2)c(F)c1]

### 3-(4-((4-((4-(4-(4-(4-(5-(2-aminopyridin-4-yl)-2-methyl-3H-imidazo[4,5-b]pyridin-3-yl)-2-fluorophenyl)piperazin-1-yl)-4-oxobutyl)piperazin-1-yl)methyl)benzyl)amino)-1-oxoisoindolin-2-yl)piperidine-2,6-dione (12)

To a 5 mL MWV equipped with a magnetic stir bar containing To a 8 mL MWV equipped with a magnetic stir bar containing 4-(3-(3-fluoro-4-(piperazin-1-yl)phenyl)-2-methyl-3H-imidazo[4,5-b]pyridin-5-yl)pyridin-2-amine (A7) (18 mg, 45 μmol, 1.2 eq.), 4-(4-(4-(((2-(2,6-dioxopiperidin-3-yl)-1-oxoisoindolin-4-yl)amino)methyl)benzyl)piperazin-1-yl) butanoic acid (12b) (24 mg, 45 μmol, 1.0 eq.), and HATU (19 mg, 49 μmol, 1.3 eq.) was added DMF (0.75 mL) and DIPEA (35 mg, 0.27 mmol, 7.3 eq.) sequentially. The reaction mixture was stirred at ambient temperature for 24 hours where it was then concentrated under reduced pressure and purified by flash column chromatography to give 3-(4-((4-((4-(4-(4-(4-(5-(2-aminopyridin-4-yl)-2-methyl-3H-imidazo[4,5-b]pyridin-3-yl)-2-fluorophenyl)piperazin-1-yl)-4-oxobutyl)piperazin-1-yl)methyl)benzyl)amino)-1-oxoisoindolin-2-yl)piperidine-2,6-dione (12) (12.5 mg, 13.6 μmol, 37% yield, eluting at 5% MeOH in DCM). ¹H NMR (500 MHz, MeOD) δ 8.41 (s, 1H), 8.06 (d, J=8.4 Hz, 1H), 7.97 (d, J=8.3 Hz, 1H), 7.85-7.76 (m, 2H), 7.38-7.14 (m, 6H), 7.14-7.07 (m, 2H), 6.96 (d, J=7.5 Hz, 1H), 6.59 (d, J=8.1 Hz, 1H), 5.12-5.01 (m, 1H), 4.36 (s, 2H), 4.29-4.17 (m, 2H), 3.69 (s, 3H), 3.50 (s, 2H), 3.25 (s, 2H), 3.16-3.03 (m, 4H), 2.87-2.76 (m, 1H), 2.73-2.29 (m, 14H), 2.12-2.04 (m, 2H), 1.82-1.75 (m, 2H), 0.86-0.63 (m, 2H). LCMS (ESI) C₅₁H₅₅FN₁₂₀₄ requires 919.44, found 920 (M/Z+H).

### Example 13. Synthesis of 5-((14-(4-(4-(5-(2-aminopyridin-4-yl)-2-methyl-3H-imidazo[4,5-b]pyridin-3-yl)-2-fluorophenyl)piperazin-1-yl)-3,6,9,12-tetraoxatetradecyl)amino)-2-(2,6-dioxopiperidin-3-yl)-6-fluoroisoindoline-1,3-dione (13)

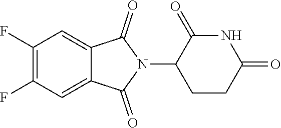 [SMILES: O=C1CCC(N2C(=O)c3cc(F)c(F)cc3C2=O)C(=O)N1]

### 2-(2,6-dioxopiperidin-3-yl)-5,6-difluoroisoindoline-1,3-dione (D)

Acetic acid (18.00 mL) was added to a 250 mL RBF equipped with a stir bar containing 3-aminopiperidine-2,6-dione, HCl (894 mg, 5.43 mmol, 1.0 eq.), 5,6-difluoroisobenzofuran-1,3-dione (1.00 g, 5.43 mmol, 1.0 eq.), 3-aminopiperidine-2,6-dione, HCl (894 mg, 5.43 mmol, 1.0 eq.), and was stirred at 120 C overnight, cooled, and concentratred under reduced pressure. The residue was suspended in D.I. water (100 mL) and stirred for 3 hours and filtered over a fritted funnel. The solid was dissolved and concentrated under reduced pressure. The residue was washed with hexanes and filtered to give 2-(2,6-dioxopiperidin-3-yl)-5,6-difluoroisoindoline-1,3-dione (D) (1.25 g, 4.27 mmol, 78% yield), a purple solid. ¹H NMR (500 MHz, DMSO) δ 11.15 (s, 1H), 8.16 (t, J=7.6 Hz, 2H), 5.18 (dd, J=12.9, 5.4 Hz, 1H), 2.90 (ddd, J=17.1, 13.9, 5.4 Hz, 1H), 2.62 (ddd, J=17.2, 4.6, 2.4 Hz, 1H), 2.58-2.46 (m, 1H), 2.07 (dtd, J=13.0, 5.4, 2.3 Hz, 1H). ¹³C NMR (126 MHz, DMSO) δ 172.70, 169.64, 165.37, 153.80 (dd, J=257.9, 15.4 Hz), 128.57 (t, J=5.8 Hz), 113.91 (dd, J=14.7, 6.4 Hz), 49.36, 30.89, 21.89.

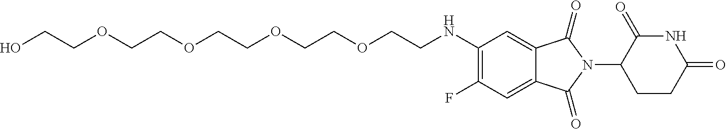 [SMILES: O=C1CCC(N2C(=O)c3cc(F)c(NCCOCCOCCOCCOCCO)cc3C2=O)C(=O)N1]

### 2-(2,6-dioxopiperidin-3-yl)-5-fluoro-6-((14-hydroxy-3,6,9,12-tetraoxatetradecyl)amino)isoindoline-1,3-dione (13a)

To a 5 mL MWV equipped with a stir bar was added 14-amino-3,6,9,12-tetraoxatretradecan-1-ol (63 mg, 0.27 mmol, 1.0 eq.), N-ethyl-N-isopropylpropan-2-amine (0.28 mL, 1.6 mmol, 6.0 eq.), and 1,4-dioxane (0.90 mL). The mixture was stirred for 5 minutes at room temperature under an argon atmosphere where 2-(2,6-dioxopiperidin-3-yl)-5,6-dilfuoroisoindoline-1,3-dione (D) (94 mg, 0.32 mol, 1.2 eq.) was added and the reaction mixture was stirred at 115 C for 24 hours. The reaction was mixture was cooled to room temperature, concentrated under reduced pressure and purified by automated column chromatography using a CombiFlash to afford 2-(2,6-dioxopiperidin-3-yl)-5-fluoro-6-((14-hydroxy-3,6,9,12-tetraoxatetradecyl)amino)isoindoline-1,3-dione (13a) (35 mg, 0.06 mmol, 26% yield), a green solid. ¹H NMR (500 MHz, MeOD) δ 7.31 (d, J=10.2 Hz, 1H), 7.12 (d, J=7.2 Hz, 1H), 4.94 (dd, J=12.8, 5.4 Hz, 1H), 3.61 (t, J=5.4 Hz, 2H), 3.57-3.48 (m, 14H), 3.46-3.36 (m, 5H), 3.21 (p, J=1.7 Hz, 1H), 2.76 (ddd, J=17.4, 13.9, 5.3 Hz, 1H), 2.68-2.54 (m, 2H), 2.00 (dtd, J=13.0, 5.7, 2.7 Hz, 1H). ¹³C NMR (126 MHz, MeOD) δ 174.66, 171.58, 168.92, 168.55 (d, J=2.8 Hz), 156.04, 154.07, 144.59 (d, J=12.8 Hz), 131.47 (d, J=2.3 Hz), 118.93 (d, J=8.7 Hz), 110.61 (d, J=22.5 Hz), 106.74 (d, J=5.7 Hz), 73.63, 71.60, 71.58, 71.55, 71.52, 71.50, 71.34, 62.20, 50.52, 43.80, 32.21, 23.80.

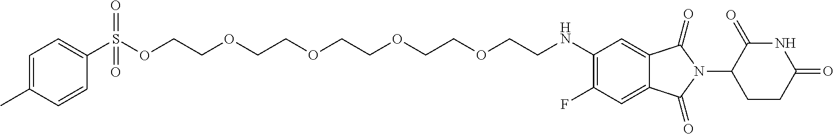 [SMILES: Cc1ccc(S(=O)(=O)OCCOCCOCCOCCOCCNc2cc3c(cc2F)C(=O)N(C2CCC(=O)NC2=O)C3=O)cc1]

### 14-((2-(2,6-dioxopiperidin-3-yl)-6-fluoro-1,3-dioxoisoindolin-5-yl)amino)-3,6,9,12-tetraoxatetradecyl 4-methylbenzenesulfonate (13b)

In a 8 mL MWV equipped with a stir bar containing 2-(2,6-dioxopiperidin-3-yl)-5-fluoro-6-((14-hydroxy-3,6,9,12-tetraoxatetradecyl)amino)isoindoline-1,3-dione (13a) (350 mg, 0.06 mmol, 1.0 eq.), DCM (1.50 mL), and triethylamine (21 mg, 0.21 mmol, 3.0 eq.) was added 4-toluenesulfonyl chloride (16 mg, 0.08 mmol, 1.2 eq.) at 0 C. The reaction mixture was allowed to warm up to room temperature and stirred for 3 hours. The reaction mixture was diluted with water and extracted with DCM (3×20 mL). The organic layers were recombined, dried over sodium sulfate, and concentrated under reduced pressure. The crude residue was purified by column chromatography using a CombiFlash to give 14-((2-(2,6-dioxopiperidin-3-yl)-6-fluoro-1,3-dioxoisoindolin-5-yl)amino)-3,6,9,12-tetraoxatetradecyl 4-methylbenzenesulfonate (13b) (15 mg, 0.02 mmol, 33% yield, eluting at 5% MeOH in DCM), a yellow semisolid. ¹H NMR (500 MHz, CDCl₃) δ 8.09 (s, 1H), 7.79 (d, J=8.3 Hz, 2H), 7.39 (d, J=9.8 Hz, 1H), 7.33 (d, J=8.1 Hz, 2H), 7.09 (d, J=7.1 Hz, 1H), 4.91 (dd, J=12.3, 5.3 Hz, 1H), 4.14 (dd, J=5.6, 4.0 Hz, 2H), 3.76 (t, J=5.1 Hz, 2H), 3.71-3.59 (m, 10H), 3.58 (s, 4H), 3.56 (s, 1H), 3.44 (t, J=5.1 Hz, 2H), 2.92-2.67 (m, 3H), 2.44 (s, 3H), 2.17-2.09 (m, 1H). ¹³C NMR (126 MHz, CDCl₃) δ 171.05, 168.34, 167.52, 166.99 (dd, J=3608.4, 2.6 Hz), 154.97, 152.99, 144.95, 142.82 (d, J=12.7 Hz), 133.12, 130.18 (d, J=2.5 Hz), 129.96, 128.11, 118.72 (d, J=8.8 Hz), 110.25 (d, J=22.3 Hz), 105.87 (d, J=5.3 Hz), 70.89, 70.78, 70.76, 70.69, 70.64, 69.36, 68.94, 68.83, 49.40, 43.04, 31.57, 22.88, 21.78.

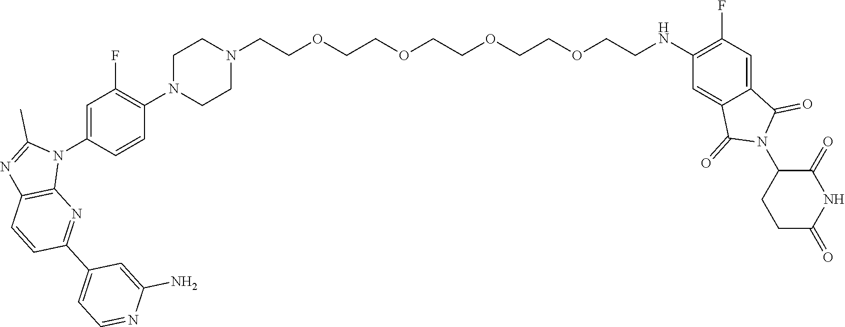 [SMILES: Cc1nc2ccc(-c3ccnc(N)c3)nc2n1-c1ccc(N2CCN(CCOCCOCCOCCOCCNc3cc4c(cc3F)C(=O)N(C3CCC(=O)NC3=O)C4=O)CC2)c(F)c1]

### 5-((14-(4-(4-(5-(2-aminopyridin-4-yl)-2-methyl-3H-imidazo[4,5-b]pyridin-3-yl)-2-fluorophenyl)piperazin-1-yl)-3,6,9,12-tetraoxatetradecyl)amino)-2-(2,6-dioxopiperidin-3-yl)-6-fluoroisoindoline-1,3-dione (13)

1,4-Dioxane (0.85 mL) and DIPEA (8.4 mg, 65 μmol, 3.0 eq.) were added to a 5 mL MWV equipped with a magnetic stir bar containing 4-(3-(3-fluoro-4-(piperazin-1-yl)phenyl)-2-methyl-3H-imidazo[4,5-b]pyridin-5-yl)pyridin-2-amine (A7) (8.7 mg, 22 μmol, 1.0 eq.), 14-((2-(2,6-dioxopiperidin-3-yl)-6-fluoro-1,3-dioxoisoindolin-5-yl)amino)-3,6,9,12-tetraoxatetradecyl 4-methylbenzenesulfonate (13b) (15 mg, 23 μmol, 1.0 eq.), and sodium iodide (1.3 mg, 8.6 μmol, 0.4 eq.). The reaction mixture was stirred at 90° C. for 16 hours where it was then cooled down to room temperature, concentrated under reduced pressure, and purified by flash column chromatography to give 5-((14-(4-(4-(5-(2-aminopyridin-4-yl)-2-methyl-3H-imidazo[4,5-b]pyridin-3-yl)-2-fluorophenyl)piperazin-1-yl)-3,6,9,12-tetraoxatetradecyl)amino)-2-(2,6-dioxopiperidin-3-yl)-6-fluoroisoindoline-1,3-dione (13) (6 mg, 7 μmol, 30% yield), a white solid. LCMS (ESI) C₄₅H₅₀F2N₁₀O₈ requires 896.95, found 897 (M/Z+H). ¹H NMR (500 MHz, CDCl₃) δ 8.05-7.99 (m, 2H), 7.86-7.79 (m, 1H), 7.75 (dd, J=8.3, 3.2 Hz, 1H), 7.36-7.28 (m, 2H), 7.23 (dd, J=6.2, 1.6 Hz, 1H), 7.20-7.10 (m, 3H), 7.03 (d, J=7.1 Hz, 1H), 6.28 (s, 2H), 5.42 (q, J=5.0 Hz, 1H), 4.84 (dd, J=12.1, 5.4 Hz, 1H), 3.82 (s, 2H), 3.68 (dt, J=13.4, 5.9 Hz, 3H), 3.62 (s, 13H), 3.10 (q, J=7.4 Hz, 4H), 2.78-2.62 (m, 5H), 2.55 (s, 3H), 2.11-1.99 (m, 1H), 1.48 (t, J=7.4 Hz, 3H), 1.35 (d, J=6.4 Hz, 1H).

### Example 14. Synthesis of 3-(4-((4-((4-(5-(4-(4-(5-(2-aminopyridin-4-yl)-2-methyl-3H-imidazo[4,5-b]pyridin-3-yl)-2-fluorophenyl)piperazin-1-yl)-5-oxopentyl)piperazin-1-yl)methyl)benzyl)amino)-1-oxoisoindolin-2-yl)piperidine-2,6-dione (14)

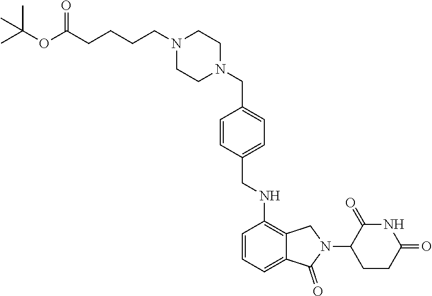 [SMILES: CC(C)(C)OC(=O)CCCCN1CCN(Cc2ccc(CNc3cccc4c3CN(C3CCC(=O)NC3=O)C4=O)cc2)CC1]

### tert-butyl 5-(4-(4-(((2-(2,6-dioxopiperidin-3-yl)-1-oxoisoindolin-4-yl)amino)methyl)benzyl)piperazin-1-yl)pentanoate (14a)

To a 8 mL equipped with a magnetic stir bar containing 3-(1-oxo-4-((4-(piperazin-1-ylmethyl)benzyl)amino)isoindolin-2-yl)piperidine-2,6-dione, HCl (10c) (30 mg, 62 μmol, 1.0 eq.) in acetonitrile (1.00 mL) and DMF (1.00 mL) was added tert-butyl 5-bromopentanoate (15 mg, 65 μmol, 1.05 eq.), potassium iodide (2.1 mg, 12 μmol, 0.2 eq.), and DIPEA (40 mg, 0.31 mmol, 5.0 eq.). The reaction mixture was stirred at 115° C. for 16 hours where it was concentrated under reduced pressure and purified by flash column chromatography to give tert-butyl 5-(4-(4-(((2-(2,6-dioxopiperidin-3-yl)-1-oxoisoindolin-4-yl)amino)methyl)benzyl)piperazin-1-yl)pentanoate (14a) (22 mg, 36 μmol, 59% yield). LCMS (ESI) C₃₄H₄₅N₅O₅ requires 603.76, found 604.25 (M/Z+H).

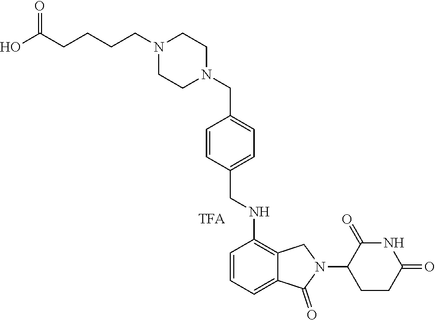 [SMILES: O=C(O)CCCCN1CCN(Cc2ccc(CNc3cccc4c3CN(C3CCC(=O)NC3=O)C4=O)cc2)CC1]

### 5-(4-(4-(((2-(2,6-dioxopiperidin-3-yl)-1-oxoisoindolin-4-yl)amino)methyl)benzyl)piperazin-1-yl)pentanoic acid, Trifluoroacetic acid (14b)

To a 5 mL MWV equipped with a magnetic stir bar containing tert-butyl 5-(4-(4-(((2-(2,6-dioxopiperidin-3-yl)-1-oxoisoindolin-4-yl)amino)methyl)benzyl)piperazin-1-yl)pentanoate (14a) (23 mg, 38 μmol, 1.0 eq.) in DCM (0.40 mL) was added trifluoroacetic acid (0.10 mL, 1.3 mmol, 34 eq.). The reaction mixture was stirred at room temperature for 16 hours where it was concentrated under reduced pressure to give 5-(4-(4-(((2-(2,6-dioxopiperidin-3-yl)-1-oxoisoindolin-4-yl)amino)methyl)benzyl)piperazin-1-yl)pentanoic acid, Trifluoroacetic acid (14b) in quantitative yield and was used in the next step without further purification. LCMS (ESI) C₃₀H₃₇N₅O₅ 547.66, found 548 (M/Z+H).

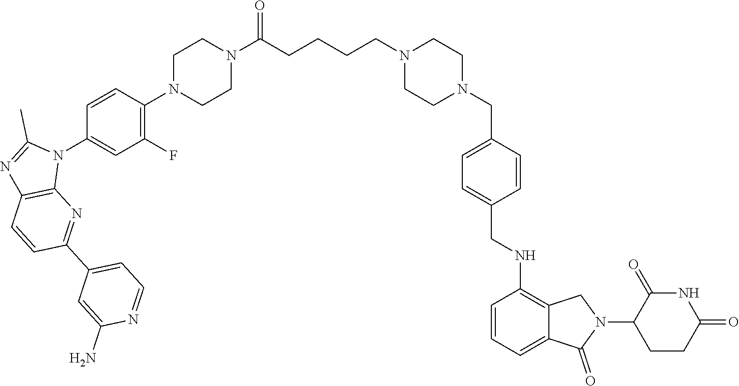 [SMILES: Cc1nc2ccc(-c3ccnc(N)c3)nc2n1-c1ccc(N2CCN(C(=O)CCCCN3CCN(Cc4ccc(CNc5cccc6c5CN(C5CCC(=O)NC5=O)C6=O)cc4)CC3)CC2)c(F)c1]

### 3-(4-((4-((4-(5-(4-(4-(5-(2-aminopyridin-4-yl)-2-methyl-3H-imidazo[4,5-b]pyridin-3-yl)-2-fluorophenyl)piperazin-1-yl)-5-oxopentyl)piperazin-1-yl)methyl)benzyl)amino)-1-oxoisoindolin-2-yl)piperidine-2,6-dione (14)

DMF (0.50 mL) and DIPEA (29 mg, 0.23 mmol, 6.0 eq.) were added to an 5 mL MWV equipped with a magnetic stir bar containing 4-(3-(3-fluoro-4-(piperazin-1-yl)phenyl)-2-methyl-3H-imidazo[4,5-b]pyridin-5-yl)pyridin-2-amine (A7) (15 mg, 38 μmol, 1.0 eq.), 5-(4-(4-(((2-(2,6-dioxopiperidin-3-yl)-1-oxoisoindolin-4-yl)amino)methyl)benzyl)piperazin-1-yl)pentanoic acid, Trifluoroacetic acid (14b) (25 mg, 38 μmol, 1.0 eq.), and HATU (16 mg, 42 μmol, 1.1 eq.). The reaction mixture was stirred overnight at room temperature and was concentrated under reduced pressure. The residue was purified by flash column chromatography to give 3-(4-((4-((4-(5-(4-(4-(5-(2-aminopyridin-4-yl)-2-methyl-3H-imidazo[4,5-b]pyridin-3-yl)-2-fluorophenyl)piperazin-1-yl)-5-oxopentyl)piperazin-1-yl)methyl)benzyl)amino)-1-oxoisoindolin-2-yl)piperidine-2,6-dione (14) (18 mg, 16 μmol, 43% yield, 85% Purity). LCMS (ESI) C₅₂H₅₇FN_{1204 933.11}, found 467.19.

### Example 15. Synthesis of 3-(4-((4-((4-(6-(4-(4-(5-(2-aminopyridin-4-yl)-2-methyl-3H-imidazo[4,5-b]pyridin-3-yl)-2-fluorophenyl)piperazin-1-yl)-6-oxohexyl)piperazin-1-yl)methyl)benzyl)amino)-1-oxoisoindolin-2-yl)piperidine-2,6-dione (15)

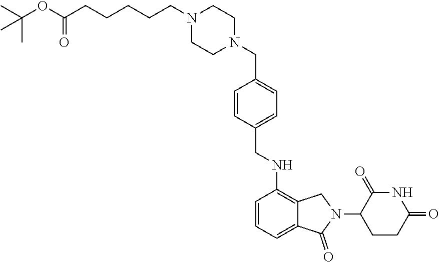 [SMILES: CC(C)(C)OC(=O)CCCCCN1CCN(Cc2ccc(CNc3cccc4c3CN(C3CCC(=O)NC3=O)C4=O)cc2)CC1]

### tert-butyl 6-(4-(4-(((2-(2,6-dioxopiperidin-3-yl)-1-oxoisoindolin-4-yl)amino)methyl)benzyl)piperazin-1-yl) hexanoate (15a)

To a 5 mL MWV equipped with a magnetic stir bar containing 3-(1-oxo-4-((4-(piperazin-1-ylmethyl)benzyl)amino)isoindolin-2-yl)piperidine-2,6-dione, HCl (10c) (30 mg, 62 μmol, 1.0 eq.), in DMF (1.00 mL) was added tert-butyl 6-bromohexanoate (19 mg, 76 μmol, 1.2 eq.), potassium iodide (2.6 mg, 16 μmol, 0.25 eq.), and DIPEA (24 mg, 0.19 mmol, 3.0 eq.). The reaction mixture was stirred for 16 hours at 80 C. After completion of the reaction, the reaction mixture was concentrated under reduce pressure and purified by flash column chromatography to give tert-butyl 6-(4-(4-(((2-(2,6-dioxopiperidin-3-yl)-1-oxoisoindolin-4-yl)amino)methyl)benzyl)piperazin-1-yl) hexanoate (15a) (23 mg, 37 μmol, 60% yield). LCMS (ESI) C₃₅H₄₇N₅O₈ requires 617.79, found 618 (M/Z+H).

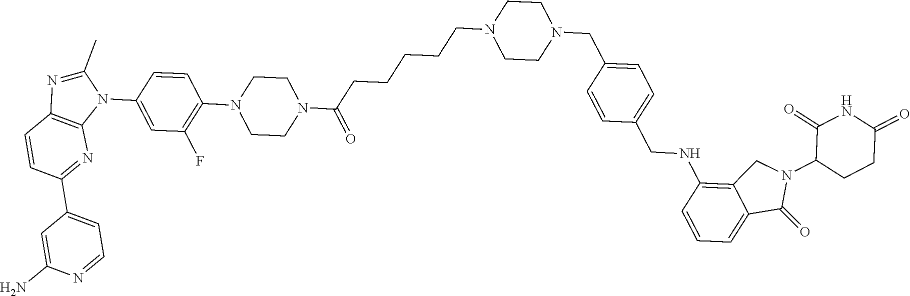 [SMILES: Cc1nc2ccc(-c3ccnc(N)c3)nc2n1-c1ccc(N2CCN(C(=O)CCCCCN3CCN(Cc4ccc(CNc5cccc6c5CN(C5CCC(=O)NC5=O)C6=O)cc4)CC3)CC2)c(F)c1]

### 3-(4-((4-((4-(6-(4-(4-(5-(2-aminopyridin-4-yl)-2-methyl-3H-imidazo[4,5-b]pyridin-3-yl)-2-fluorophenyl)piperazin-1-yl)-6-oxohexyl)piperazin-1-yl)methyl)benzyl)amino)-1-oxoisoindolin-2-yl)piperidine-2,6-dione (15)

To a 20 mL dram vial containing tert-butyl 6-(4-(4-(((2-(2,6-dioxopiperidin-3-yl)-1-oxoisoindolin-4-yl)amino)methyl)benzyl)piperazin-1-yl) hexanoate (15a) (23 mg, 23 μmol, 1.0 eq.) in DCM (0.40 mL) was added trifluoroacetic acid (0.10 mL, 1.3 mmol, 35 eq.). The reaction mixture was stirred at room temperature for 24 hours where it was concentrated under reduced pressure to give 6-(4-(4-(((2-(2,6-dioxopiperidin-3-yl)-1-oxoisoindolin-4-yl)amino)methyl)benzyl)piperazin-1-yl) hexanoic acid, Trifluoroacetic acid (15b) in quantitative yield and was used without further purification. DMF (0.50 mL) and DIPEA (29 mg, 0.23 mmol, 6.0 eq.) were added to a 5 mL MW equipped with a magnetic stir bar containing 4-(3-(3-fluoro-4-(piperazin-1-yl)phenyl)-2-methyl-3H-imidazo[4,5-b]pyridin-5-yl)pyridin-2-amine (A7) (15 mg, 38 μmol, 1.0 eq.), 5-(4-(4-(((2-(2,6-dioxopiperidin-3-yl)-1-oxoisoindolin-4-yl)amino)methyl)benzyl)piperazin-1-yl)pentanoic acid, Trifluoroacetic acid (15b) (25 mg, 38 μmol, 1.0 eq.), and HATU (16 mg, 42 μmol, 1.1 eq.). The reaction mixture was stirred at room temperature for 16 hours where it was concentrated under reduced pressure and purified by flash column chromatography to give 3-(4-((4-((4-(5-(4-(4-(5-(2-aminopyridin-4-yl)-2-methyl-3H-imidazo[4,5-b]pyridin-3-yl)-2-fluorophenyl)piperazin-1-yl)-5-oxopentyl)piperazin-1-yl)methyl)benzyl)amino)-1-oxoisoindolin-2-yl)piperidine-2,6-dione (15) (18 mg, 16 μmol, 43% yield, 85% purity), a white solid. LCMS (ESI) C₅₂H₅₇FN₁₂₀₄ requires 933.11, found 934 (M/Z+H).

### Example 16. Synthesis of 4-(((1-(1-(4-(4-(4-(5-(2-aminopyridin-4-yl)-2-methyl-3H-imidazo[4,5-b]pyridin-3-yl)-2-fluorophenyl)piperazin-1-yl)-4-oxobutyl)piperidin-4-yl)-1H-pyrazol-4-yl)methyl)amino)-2-(2,6-dioxopiperidin-3-yl)isoindoline-1,3-dione (16)

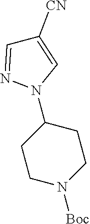 [SMILES: CC(C)(C)OC(=O)N1CCC(n2cc(C#N)cn2)CC1]

### tert-butyl 4-(4-cyano-1H-pyrazol-1-yl)piperidine-1-carboxylate (D1)

To a 8 ml MWV equipped with a magnetic stir bar was added sodium cyanide (534 mg, 10.90 mmol, 1.2 eq.) copper (I) iodide (173 mg, 0.90 mmol, 0.1 eq.), tert-butyl 4-(4-bromo-1H-pyrazol-1-yl)piperidine-1-carboxylate (3.0000 g, 9.08 mmol, 1.0 eq.), and potassium iodide (302 mg, 1.81 mmol, 0.2 eq.). The MWV was then briefly evacuated and backfilled with argon three times. Anhydrous toluene (6.20 mL) and N1,N2-dimethylethane-1,2-diamine (0.97 mL, 9.08 mmol, 1.0 eq.) were added under argon and the reaction mixture was stirred at 110 C for 24 hours. The resulting suspension was allowed to reach room temperature, diluted with 30% aqueous ammonia (5 mL), and extracted with ethyl acetate. The combined organic phases were dried over sodium sulfate, concentrated under reduced pressure, and purified by automated flash column chromatography using a CombiFlash to give tert-butyl 4-(4-cyano-1H-pyrazol-1-yl)piperidine-1-carboxylate (D1) (2.5 g, 9.0 mmol, 99% yield, eluting at 30% EtOAc in hexanes), a clear oil. NMR (500 MHz, CDCl₃) δ 7.83 (s, 1H), 7.80 (s, 1H), 4.34-4.18 (m, 3H), 2.88 (s, 2H), 2.15-2.09 (m, 2H), 1.90 (qd, J=12.3, 4.5 Hz, 2H), 1.47 (s, 9H). ¹³C NMR (126 MHz, CDCl₃) δ 154.58, 142.21, 132.07, 113.53, 92.27, 80.31, 60.37, 32.23, 31.05, 28.52. LCMS (ESI) C₁₄H₂₀N₄O₂ requires 276.15, found 277 [M+H].

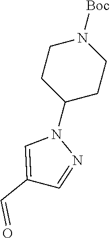 [SMILES: CC(C)(C)OC(=O)N1CCC(n2cc(C=O)cn2)CC1]

### tert-butyl 4-(4-formyl-1H-pyrazol-1-yl)piperidine-1-carboxylate (D2)

DIBAL-H (6.63 mL, 7.96 mmol, 1.1 eq. 1.2 M in hexanes) was added dropwise to a solution at −78° C. of tert-butyl 4-(4-cyano-1H-pyrazol-1-yl)piperidine-1-carboxylate (D1) (2.00 g, 7.2375 mmol, 1.0 eq.) in DCM (22.00 mL). The reaction mixture was stirred for 30 minutes at −78° C. where it was warmed to room temperature and stirred for an additional 5 hours. The reaction mixture was quenched with 1N HCl (20 mL) and stirred for 30 minutes. This was basified with solid NaHCO₃, extracted with DCM (3×25 mL), dried over anhydrous sodium sulfate, filtered, and concentrated under reduced pressure to provide tert-butyl 4-(4-formyl-1H-pyrazol-1-yl)piperidine-1-carboxylate (D2) (855 mg, 3.06 mmol, 42%), a yellow oil. ¹H NMR (400 MHz, CDCl₃) δ 9.85 (s, 1H), 7.97 (s, 1H), 7.96 (s, 1H), 4.36-4.22 (m, 3H), 2.89 (t, J=12.5 Hz, 2H), 2.20-2.10 (m, 2H), 1.90 (qd, J=12.2, 4.5 Hz, 2H), 1.47 (s, 9H). ¹³C NMR (101 MHz, CDCl₃) δ 184.14, 154.63, 140.80, 130.20, 124.29, 80.26, 77.48, 77.16, 76.84, 60.11, 42.72, 32.27, 28.53.

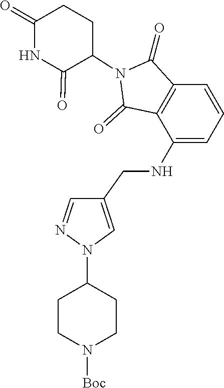 [SMILES: CC(C)(C)OC(=O)N1CCC(n2cc(CNc3cccc4c3C(=O)N(C3CCC(=O)NC3=O)C4=O)cn2)CC1]

### tert-butyl 4-(4-(((2-(2,6-dioxopiperidin-3-yl)-1,3-dioxoisoindolin-4-yl)amino)methyl)-1H-pyrazol-1-yl)piperidine-1-carboxylate (D3)

To a 5 mL MWV containing tert-butyl 4-(4-formyl-1H-pyrazol-1-yl)piperidine-1-carboxylate (123 mg, 440 μmol, 1.0 eq.), 4-amino-2-(2,6-dioxopiperidin-3-yl)isoindoline-1,3-dione (D2) (120 mg, 440 μmol, 1.0 eq.), in DMF (1.50 mL) was added dibutyldichlorostannane (134 mg, 440 μmol, 1.0 eq.) followed by phenylsilane (47 mg, 440 μmol, 1.0 eq.). The reaction mixture was heated at 80° C. for 24 hour and then concentrated under reduced pressure. The crude reside was purified by column chromatography to afford tert-butyl 4-(3-(((2-(2,6-dioxopiperidin-3-yl)-1,3-dioxoisoindolin-4-yl)amino)methyl)-1H-pyrazol-1-yl)piperidine-1-carboxylate (D3) (175 mg, 326 μmol, 74.1% yield), a yellow solid. ¹H NMR (500 MHz, CDCl₃) δ 7.50 (s, 1H), 7.49-7.45 (m, 1H), 7.41 (s, 1H), 7.12 (d, J=7.0 Hz, 1H), 6.91 (d, J=8.5 Hz, 1H), 6.42 (s, 1H), 4.93-4.86 (m, 1H), 4.35 (s, 2H), 4.30-4.21 (m, 3H), 2.91-2.68 (m, 5H), 2.15-2.08 (m, 4H), 1.86 (qd, J=12.3, 4.4 Hz, 2H), 1.45 (s, 9H). ¹³C NMR (126 MHz, CDCl₃) δ 171.18, 170.90, 169.54, 168.51, 167.63, 154.67, 146.46, 137.88, 136.31, 132.60, 125.75, 118.10, 116.95, 112.12, 110.59, 80.07, 77.41, 77.16, 76.90, 59.56, 49.05, 37.79, 32.48, 31.54, 28.52, 22.90, 21.64. LCMS (ESI) C₂₇H₃₂N₆O₆ requires 536.59, found 537 (M/Z+H).

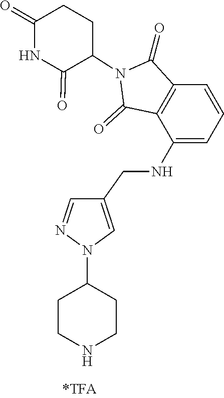 [SMILES: O=C1CCC(N2C(=O)c3cccc(NCc4cnn(C5CCNCC5)c4)c3C2=O)C(=O)N1]

### 2-(2,6-dioxopiperidin-3-yl)-4-(((1-(piperidin-4-yl)-1H-pyrazol-3-yl)methyl)amino)isoindoline-1,3-dione, Trifluoroacetic acid (D4)

4M HCl in dioxane (2.00 mL, 8.15 mmol, 25.0 eq.) was added to a 20 mL dram vial containing tert-butyl 4-(3-(((2-(2,6-dioxopiperidin-3-yl)-1,3-dioxoisoindolin-4-yl)amino)methyl)-1H-pyrazol-1-yl)piperidine-1-carboxylate (D3) (175 mg, 0.32 mmol, 1.0 eq.) in DCM (4.0 mL). The reaction mixture was stirred for 2 hours where it was concentrated under reduced pressure to give 2-(2,6-dioxopiperidin-3-yl)-4-(((1-(piperidin-4-yl)-1H-pyrazol-3-yl)methyl)amino)isoindoline-1,3-dione, Trifluoroacetic acid (D4) (146 mg, 0.26 mmol, 81% yield), an orange solid. LCMS (ESI) C₂₂H₂₄N₆O₄ requires 436.47, found 437 (M/Z+H).

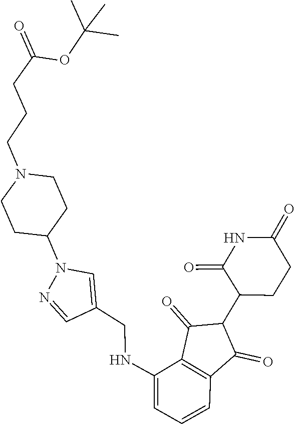 [SMILES: CC(C)(C)OC(=O)CCCN1CCC(n2cc(CNc3cccc4c3C(=O)C(C3CCC(=O)NC3=O)C4=O)cn2)CC1]

### tert-butyl 4-(4-(4-(((2-(2,6-dioxopiperidin-3-yl)-1,3-dioxoisoindolin-4-yl)amino)methyl)-1H-pyrazol-1-yl)piperidin-1-yl) butanoate (16a)

To a 8 mL MWV equipped with a stir bar containing 2-(2,6-dioxopiperidin-3-yl)-4-(((1-(piperidin-4-yl)-1H-pyrazol-4-yl)methyl)amino)isoindoline-1,3-dione, HCl (30 mg, 68 μmol, 1.0 eq.), potassium iodide (5 mg, 27 μmol, 0.4 eq.) in DMF (1.00 mL) was added tert-butyl 4-bromobutanoate (17 mg, 76 μmol, 1.1 eq.) and DIPEA (29 mg, 200 μmol, 3.0 eq.). The reaction mixture was stirred at 80° C. for 24 hours where it was then cooled to ambient temperature and concentrated under reduced pressure. The crude residue was purified by flash column chromatography to give tert-butyl 4-(4-(4-(((2-(2,6-dioxopiperidin-3-yl)-1,3-dioxoisoindolin-4-yl)amino)methyl)-1H-pyrazol-1-yl)piperidin-1-yl) butanoate (16a) (31 mg, 54 μmol, 84% yield, eluting at 28% MeOH in DCM), a yellow semisolid. ¹H NMR (500 MHz, CDCl₃) δ 9.64 (s, 2H), 8.25 (s, 1H), 7.28 (dd, J=8.5, 7.1 Hz, 1H), 7.25 (s, 1H), 6.90 (d, J=7.1 Hz, 1H), 6.71 (d, J=8.5 Hz, 1H), 6.20 (t, J=5.6 Hz, 1H), 4.70 (dd, J=12.1, 5.4 Hz, 1H), 4.13 (d, J=5.5 Hz, 2H), 3.52 (hept, J=6.7 Hz, 3H), 3.13 (s, 2H), 2.95 (q, J=7.4 Hz, 3H), 2.69-2.46 (m, 5H), 1.38 (t, J=7.5 Hz, 5H), 1.22 (s, 8H).

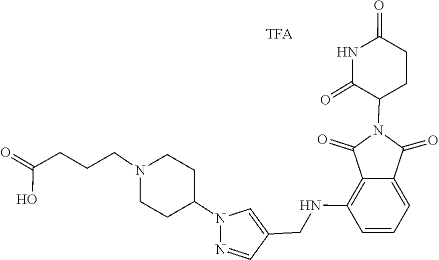 [SMILES: O=C(O)CCCN1CCC(n2cc(CNc3cccc4c3C(=O)N(C3CCC(=O)NC3=O)C4=O)cn2)CC1]

### 4-(4-(4-(((2-(2,6-dioxopiperidin-3-yl)-1,3-dioxoisoindolin-4-yl)amino)methyl)-1H-pyrazol-1-yl)piperidin-1-yl) butanoic acid, Trifluoroacetic acid (16b)

To a 20 mL dram vial containing tert-butyl 4-(4-(4-(((2-(2,6-dioxopiperidin-3-yl)-1,3-dioxoisoindolin-4-yl)amino)methyl)-1H-pyrazol-1-yl)piperidin-1-yl) butanoate (16a) (31 mg, 55 μmol, 1.0 eq.) in DCM (0.60 mL) was added trifluoroacetic acid (0.14 mL, 1.9 mmol, 35.0 eq.). The reaction mixture was stirred for 3 hours at room temperature where it was then concentrated under reduced pressure to give 4-(4-(4-(((2-(2,6-dioxopiperidin-3-yl)-1,3-dioxoisoindolin-4-yl)amino)methyl)-1H-pyrazol-1-yl)piperidin-1-yl) butanoic acid, Trifluoroacetic acid (16b) in quantitative yield. LCMS (ESI) C₃₆H₃₀N₆O₆ requires 522.56, found 523 (M/Z+H).

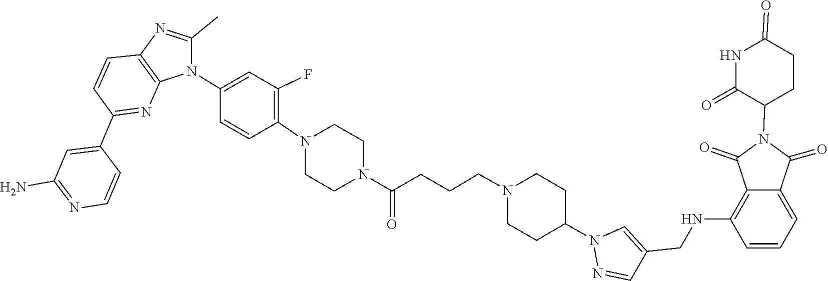 [SMILES: Cc1nc2ccc(-c3ccnc(N)c3)nc2n1-c1ccc(N2CCN(C(=O)CCCN3CCC(n4cc(CNc5cccc6c5C(=O)N(C5CCC(=O)NC5=O)C6=O)cn4)CC3)CC2)c(F)c1]

### 4-(((1-(1-(4-(4-(4-(5-(2-aminopyridin-4-yl)-2-methyl-3H-imidazo[4,5-b]pyridin-3-yl)-2-fluorophenyl)piperazin-1-yl)-4-oxobutyl)piperidin-4-yl)-1H-pyrazol-4-yl)methyl)amino)-2-(2,6-dioxopiperidin-3-yl)isoindoline-1,3-dione (16)

To a 5 mL MWV equipped with a magnetic stir bar containing 4-(3-(3-fluoro-4-(piperazin-1-yl)phenyl)-2-methyl-3H-imidazo[4,5-b]pyridin-5-yl)pyridin-2-amine (A7) (18 mg, 45 μmol, 1.0 eq.), 4-(4-(4-(((2-(2,6-dioxopiperidin-3-yl)-1,3-dioxoisoindolin-4-yl)amino)methyl)-1H-pyrazol-1-yl)piperidin-1-yl) butanoic acid, Trifluoroacetic acid (16b) (23 mg, 36 μmol, 1.0 eq.), and HATU (19 mg, 49 μmol, 1.1 eq.), was added DMF (0.75 mL) and DIPEA (35 mg, 0.27 mmol, 7.5 eq.). The reaction mixture was stirred at room temperature for 24 hours where it was concentrated under reduced pressure and purified by flash column chromatography to give 4-(((1-(1-(4-(4-(4-(5-(2-aminopyridin-4-yl)-2-methyl-3H-imidazo[4,5-b]pyridin-3-yl)-2-fluorophenyl)piperazin-1-yl)-4-oxobutyl)piperidin-4-yl)-1H-pyrazol-4-yl)methyl)amino)-2-(2,6-dioxopiperidin-3-yl)isoindoline-1,3-dione (16) (12 mg, 13 μmol, 38% yield), a yellow solid. LCMS (ESI) C₄₈H₅₀FN₁₃₀₅ requires 907.40, found 908 (M/Z+H).

### Example 17. Synthesis of 4-(((1-(1-(2-(1-(2-(4-(4-(5-(2-aminopyridin-4-yl)-2-methyl-3H-imidazo[4,5-b]pyridin-3-yl)-2-fluorophenyl)piperazin-1-yl)-2-oxoethyl)piperidin-4-yl)acetyl)piperidin-4-yl)-1H-pyrazol-4-yl)methyl)amino)-2-(2,6-dioxopiperidin-3-yl)isoindoline-1,3-dione (17)

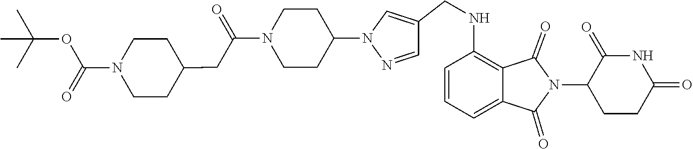 [SMILES: CC(C)(C)OC(=O)N1CCC(CC(=O)N2CCC(n3cc(CNc4cccc5c4C(=O)N(C4CCC(=O)NC4=O)C5=O)cn3)CC2)CC1]

### tert-butyl 4-(2-(4-(4-(((2-(2,6-dioxopiperidin-3-yl)-1,3-dioxoisoindolin-4-yl)amino)methyl)-1H-pyrazol-1-yl)piperidin-1-yl)-2-oxoethyl)piperidine-1-carboxylate (17a)

DMF (1.25 mL) and DIPEA (8.2 mg, 63 μmol, 1.0 eq.) were added sequentially to a 8 mL MWV equipped with a magnetic stir bar containing 2-(2,6-dioxopiperidin-3-yl)-4-(((1-(piperidin-4-yl)-1H-pyrazol-4-yl)methyl)amino)isoindoline-1,3-dione, HCl (30 mg, 63 μmol, 1.0 eq.), 2-(1-(tert-butoxycarbonyl)piperidin-4-yl)acetic acid (17 mg, 70 μmol, 1.1 eq.), and HATU (27 mg, 70 μmol, 1.1 eq.). The reaction mixture was stirred for 24 hours at room temperature where it was then concentrated under reduced pressure and purified by flash column chromatography to give tert-butyl 4-(2-(4-(4-(((2-(2,6-dioxopiperidin-3-yl)-1,3-dioxoisoindolin-4-yl)amino)methyl)-1H-pyrazol-1-yl)piperidin-1-yl)-2-oxoethyl)piperidine-1-carboxylate (17a) (34 mg, 51 μmol, 81%). ¹H NMR (500 MHz, CDCl₃) δ 8.22 (s, 1H), 7.54-7.47 (m, 2H), 7.41 (s, 1H), 7.13 (d, J=7.1 Hz, 1H), 6.93 (d, J=8.5 Hz, 1H), 4.94-4.87 (m, 1H), 4.74 (d, J=13.6 Hz, 1H), 4.36 (s, 2H), 4.35-4.28 (m, 1H), 4.08 (s, 2H), 3.99 (d, J=14.2 Hz, 1H), 3.71 (pd, J=6.6, 4.1 Hz, 2H), 3.22-3.13 (m, 3H), 2.88 (dd, J=16.0, 3.6 Hz, 1H), 2.80-2.68 (m, 5H), 2.27 (d, J=6.8 Hz, 2H), 2.21 (d, J=13.2 Hz, 1H), 2.18-2.09 (m, 1H), 2.06-1.95 (m, 1H), 1.96-1.84 (m, OH), 1.72 (d, J=12.9 Hz, 2H), 1.45 (s, 9H), 1.19-1.08 (m, 2H). ¹³C NMR (126 MHz, CDCl₃) δ 171.05, 170.24, 169.57, 168.47, 167.63, 155.00, 138.18, 136.37, 132.62, 118.23, 112.19, 79.50, 77.41, 77.16, 76.91, 59.21, 55.71, 49.07, 44.72, 43.68, 40.75, 39.68, 33.37, 33.14, 32.36, 32.18, 31.55, 29.83, 28.60, 22.93, 18.74, 17.36, 12.63. LCMS (ESI) C₃₄H₄₃N₇O₇ requires 661.32, found 662 (M/Z+H).

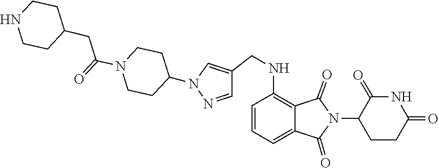 [SMILES: O=C1CCC(N2C(=O)c3cccc(NCc4cnn(C5CCN(C(=O)CC6CCNCC6)CC5)c4)c3C2=O)C(=O)N1]

### 2-(2,6-dioxopiperidin-3-yl)-4-(((1-(1-(2-(piperidin-4-yl)acetyl)piperidin-4-yl)-1H-pyrazol-4-yl)methyl)amino)isoindoline-1,3-dione, Trifluoroacetic acid (17b)

To a 20 mL dram vial containing tert-butyl 4-(2-(4-(4-(((2-(2,6-dioxopiperidin-3-yl)-1,3-dioxoisoindolin-4-yl)amino)methyl)-1H-pyrazol-1-yl)piperidin-1-yl)-2-oxoethyl)piperidine-1-carboxylate (34 mg, 51 μmol, 1.0 eq.) in DCM (0.65 mL) was added TFA (0.14 mL, 1.8 mmol, 35.0 eq.). The reaction mixture was stirred for 3 hours at room temperature where it was then concentrated under reduced pressure to give 2-(2,6-dioxopiperidin-3-yl)-4-(((1-(1-(2-(piperidin-4-yl)acetyl)piperidin-4-yl)-1H-pyrazol-4-yl)methyl)amino)isoindoline-1,3-dione, Trifluoroacetic acid (17b) (25 mg, 37 μmol, 72% yield), a yellow semisolid and was used in the next step without purification. ¹H NMR (500 MHz, CDCl₃) δ 9.66 (s, 2H), 8.85 (d, J=3.1 Hz, 1H), 7.52-7.45 (m, 2H), 7.43 (s, 1H), 7.10 (d, J=7.0 Hz, 1H), 6.91 (d, J=8.5 Hz, 1H), 4.89 (dd, J=12.1, 5.4 Hz, 1H), 4.68 (d, J=13.7 Hz, 1H), 3.69-3.57 (m, 3H), 3.11-3.05 (m, 3H), 2.95-2.79 (m, 2H), 2.80-2.66 (m, 3H), 2.32 (d, J=6.7 Hz, 2H), 2.23-2.07 (m, 5H), 1.99-1.78 (m, 5H), 1.56 (q, J=12.4 Hz, 2H). LCMS (ESI) C₂₉H₃₅N₇O₅ requires 561.26, found 562 (M/Z+H).

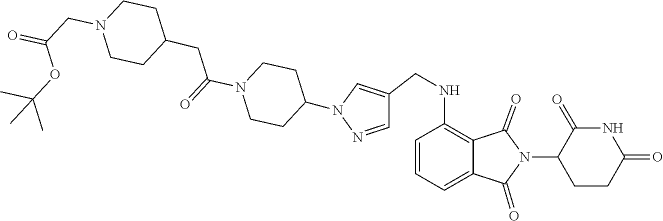 [SMILES: CC(C)(C)OC(=O)CN1CCC(CC(=O)N2CCC(n3cc(CNc4cccc5c4C(=O)N(C4CCC(=O)NC4=O)C5=O)cn3)CC2)CC1]

### tert-butyl 2-(4-(2-(4-(4-(((2-(2,6-dioxopiperidin-3-yl)-1,3-dioxoisoindolin-4-yl)amino)methyl)-1H-pyrazol-1-yl)piperidin-1-yl)-2-oxoethyl)piperidin-1-yl)acetate (17c)

DMF (1.00 mL), DIPEA (17 mg, 0.13 mmol, 3.5 eq.), and tert-butyl 2-bromoacetate (8.7 mg, 0.044 mmol, 1.2 eq.) were added sequentially to a 5 mL MWV equipped with a magnetic stir bar containing 2-(2,6-dioxopiperidin-3-yl)-4-(((1-(1-(2-(piperidin-4-yl)acetyl)piperidin-4-yl)-1H-pyrazol-4-yl)methyl)amino)isoindoline-1,3-dione, trifluoroacetic acid (17b) (25 mg, 0.037 mmol,1.0 eq.) and potassium iodide (2.5 mg, 0.015 mmol, 0.4 eq.). The reaction mixture was stirred at 80° C. for 24 hours. It was allowed to cool to ambient temperature and was concentrated under reduced pressure. The crude residue was purified by flash column chromatography to give tert-butyl 2-(4-(2-(4-(4-(((2-(2,6-dioxopiperidin-3-yl)-1,3-dioxoisoindolin-4-yl)amino)methyl)-1H-pyrazol-1-yl)piperidin-1-yl)-2-oxoethyl)piperidin-1-yl)acetate, DIPEA (17c) (29 mg, 0.034 mmol, 93% yield, eluting at 7% MeOH in DCM), a yellow semisolid. ¹H NMR (500 MHz, CDCl₃) δ 8.53 (d, J=6.5 Hz, 1H), 7.46 (s, 1H), 7.41 (s, 1H), 7.09 (d, J=7.1 Hz, 1H), 6.91 (d, J=8.5 Hz, 1H), 6.39 (t, J=5.6 Hz, 1H), 4.93-4.85 (m, 1H), 4.67 (d, J=13.8 Hz, 1H), 4.34 (d, J=5.5 Hz, 2H), 4.29 (dt, J=11.7, 4.2 Hz, 1H), 3.97 (d, J=13.9 Hz, 1H), 3.48 (s, 2H), 3.30 (d, J=11.1 Hz, 2H), 2.93 (s, 1H), 2.88-2.78 (m, 4H), 2.78-2.65 (m, 3H), 2.32 (d, J=6.8 Hz, 2H), 2.17 (d, J=12.8 Hz, 1H), 2.14-2.02 (m, 1H), 1.96-1.81 (m, 4H), 1.72 (q, J=13.5 Hz, 2H), 1.45 (s, 9H). ¹³C NMR (126 MHz, CDCl₃) δ 171.23, 169.73, 169.49, 168.57, 167.62, 162.67, 146.46, 138.21, 136.30, 132.52, 125.86, 118.04, 117.03, 112.02, 110.48, 59.02, 57.94, 54.43, 52.98, 48.99, 47.81, 44.56, 42.65, 40.72, 38.75, 37.74, 36.60, 32.98, 32.18, 31.51, 31.18, 30.38, 29.75, 28.14, 22.86, 18.98, 18.74, 17.46, 12.27.

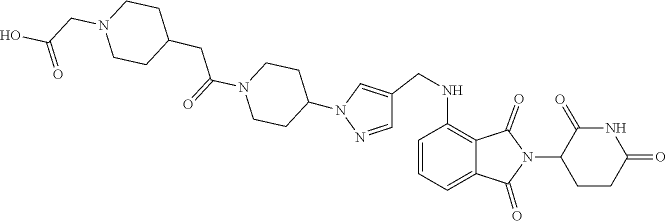 [SMILES: O=C(O)CN1CCC(CC(=O)N2CCC(n3cc(CNc4cccc5c4C(=O)N(C4CCC(=O)NC4=O)C5=O)cn3)CC2)CC1]

### 2-(4-(2-(4-(4-(((2-(2,6-dioxopiperidin-3-yl)-1,3-dioxoisoindolin-4-yl)amino)methyl)-1H-pyrazol-1-yl)piperidin-1-yl)-2-oxoethyl)piperidin-1-yl)acetic acid, Trifluoroacetic acid (17d)

To a 20 mL dram vial containing tert-butyl 2-(4-(2-(4-(4-(((2-(2,6-dioxopiperidin-3-yl)-1,3-dioxoisoindolin-4-yl)amino)methyl)-1H-pyrazol-1-yl)piperidin-1-yl)-2-oxoethyl)piperidin-1-yl)acetate (17c) (29 mg, 43 μmol, 1.0 eq.) in DCM (0.60 mL) was added TFA (0.12 mL, 1.5 mmol, 35.0 eq.). The reaction mixture was stirred for 24 hours at room temperature where it was diluted with DCM, concentrated under reduced pressure and purified by flash column chromatography to give tert-butyl 2-(4-(2-(4-(4-(((2-(2,6-dioxopiperidin-3-yl)-1,3-dioxoisoindolin-4-yl)amino)methyl)-1H-pyrazol-1-yl)piperidin-1-yl)-2-oxoethyl)piperidin-1-yl)acetate, Trifluoroacetic acid (17d) (29 mg, 43 μmol, 92% yield, eluting at 7% MeOH in DCM), a yellow semisolid. LCMS (ESI) C₃₁H₃₇N₇O₇ requires 619.27, found 620 (M/Z+H).

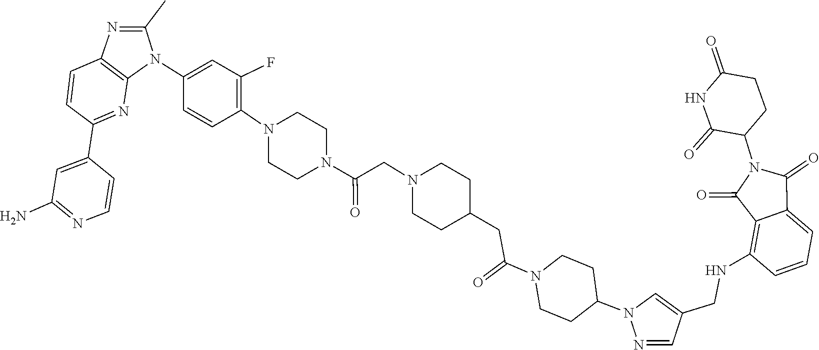 [SMILES: Cc1nc2ccc(-c3ccnc(N)c3)nc2n1-c1ccc(N2CCN(C(=O)CN3CCC(CC(=O)N4CCC(n5cc(CNc6cccc7c6C(=O)N(C6CCC(=O)NC6=O)C7=O)cn5)CC4)CC3)CC2)c(F)c1]

### 4-(((1-(1-(2-(1-(2-(4-(4-(5-(2-aminopyridin-4-yl)-2-methyl-3H-imidazo[4,5-b]pyridin-3-yl)-2-fluorophenyl)piperazin-1-yl)-2-oxoethyl)piperidin-4-yl)acetyl)piperidin-4-yl)-1H-pyrazol-4-yl)methyl)amino)-2-(2,6-dioxopiperidin-3-yl)isoindoline-1,3-dione (17)

To a 8 mL MWV equipped with a magnetic stir bar containing 4-(3-(3-fluoro-4-(piperazin-1-yl)phenyl)-2-methyl-3H-imidazo[4,5-b]pyridin-5-yl)pyridin-2-amine (A7) (14 mg, 35 μmol, 1.2 eq.), 2-(4-(2-(4-(4-(((2-(2,6-dioxopiperidin-3-yl)-1,3-dioxoisoindolin-4-yl)amino)methyl)-1H-pyrazol-1-yl)piperidin-1-yl)-2-oxoethyl)piperidin-1-yl)acetic acid, trifluoroacetic acid (17d) (22 mg, 30 μmol, 1.0 eq.), and HATU (15 mg, 38 μmol, 1.3 eq.) was added DMF (0.75 mL) and DIPEA (27 mg, 0.21 mmol, 7.0 eq.) sequentially. The reaction mixture was stirred at ambient temperature for 24 hours where it was concentrated under reduced pressure and purified by flash column chromatography to give 4-(((1-(1-(2-(1-(2-(4-(4-(5-(2-aminopyridin-4-yl)-2-methyl-3H-imidazo[4,5-b]pyridin-3-yl)-2-fluorophenyl)piperazin-1-yl)-2-oxoethyl)piperidin-4-yl)acetyl)piperidin-4-yl)-1H-pyrazol-4-yl)methyl)amino)-2-(2,6-dioxopiperidin-3-yl)isoindoline-1,3-dione (17) (16.5 mg, 14 μmol, 42% yield, 88% Purity, 48% yield). LCMS (ESI) C₅₃H₅₇FN₁₄₀₆ requires 1004.45, found 1005 (M/Z+H).

### Example 18. Synthesis of 4-(((1-(1-(1-(5-(3-(4-(4-(5-(2-aminopyridin-4-yl)-2-methyl-3H-imidazo[4,5-b]pyridin-3-yl)-2-fluorophenyl)piperazin-1-yl)prop-1-yn-1-yl)pyridin-2-yl)piperidine-4-carbonyl)piperidin-4-yl)-1H-pyrazol-4-yl)methyl)amino)-2-(2,6-dioxopiperidin-3-yl)isoindoline-1,3-dione (18)

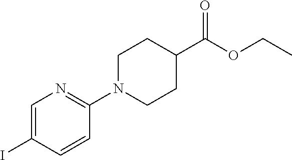 [SMILES: CCOC(=O)C1CCN(c2ccc(I)cn2)CC1]

### ethyl 1-(5-iodopyridin-2-yl)piperidine-4-carboxylate (D5)

To a solution of DMF (7.70 mL), potassium carbonate (879 mg, 6.3609 mmol, 1.0 eq.), and ethyl piperidine-4-carboxylate (0.98 mL, 6.36 mmol, 1.0 eq.) was added 2-fluoro-5-iodopyridine (1.70 g, 7.63 mmol, 1.2 eq.). The MWV was capped and stirred at 110° C. for 24 hours. After stirring, the mixture was cooled to room temperature and extracted with ethyl acetate washed with distilled water (3×) and brine (1×). The combined extracts were dried over Na₂SO₄ and concentrated under reduced pressure. The residue was purified by flash column chromatography to give ethyl 1-(5-iodopyridin-2-yl)piperidine-4-carboxylate (D5) (1.98 g, 5.50 mmol, 86%), a clear liquid. LCMS (ESI) C₁₃H₁₇IN₂O₂ requires 360.03, found 361 (M/Z+H).

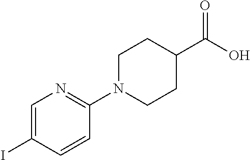 [SMILES: O=C(O)C1CCN(c2ccc(I)cn2)CC1]

### 1-(5-iodopyridin-2-yl)piperidine-4-carboxylic acid (D6)

To a solution of ethyl 1-(5-iodopyridin-2-yl)piperidine-4-carboxylate (D5) (1.98 g, 5.50 mmol, 1.0 eq.) in THF (41.2 mL) and Methanol (13.8 mL) was added lithium hydroxide, water (461 mg, 11.01 mmol, 2.0 eq.). The resulting mixture was stirred at ambient temperature for 16 hours.

Water (50 mL) was added and the reaction mixture was concentrated under reduced pressure. Water (50 ml) and ether (100 mL) were added and the solution was stirred for 10 minutes. The ethereal layer was removed and the aqueous layer acidified to pH 2 with 1N HCl at 0° C. and stirred for 20 minutes. The white precipitate was collected by filtration and washed with ice-cold water to give 1-(5-iodopyridin-2-yl)piperidine-4-carboxylic acid (D6) (245.4 mg, 738.8 μmol, 13.43%), a white solid. LCMS (ESI) C₁₁H₁₃IN₂O₂ requires 332.00, found 333 (M/Z+H).

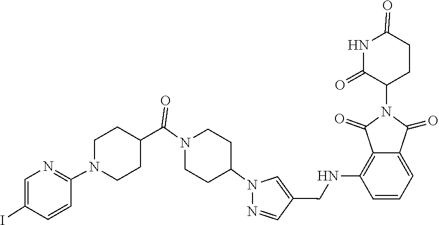 [SMILES: O=C1CCC(N2C(=O)c3cccc(NCc4cnn(C5CCN(C(=O)C6CCN(c7ccc(I)cn7)CC6)CC5)c4)c3C2=O)C(=O)N1]

### 2-(2,6-dioxopiperidin-3-yl)-4-(((1-(1-(1-(5-iodopyridin-2-yl)piperidine-4-carbonyl)piperidin-4-yl)-1H-pyrazol-4-yl)methyl)amino)isoindoline-1,3-dione (D7)

DMF (5.8 mL) and DIPEA (0.60 mL, 3.47 mmol, 6.0 eq.) were added sequentially to a 20 mL MWV equipped with a magnetic stir bar containing HATU (242 mg, 0.63 mmol, 1.1 eq.), 2-(2,6-dioxopiperidin-3-yl)-4-(((1-(piperidin-4-yl)-1H-pyrazol-4-yl)methyl)amino)isoindoline-1,3-dione, Trifluoroacetic acid (D4) (424 mg 0.57 mmol, 1.0 eq., 75% Wt.), and 1-(5-iodopyridin-2-yl)piperidine-4-carboxylic acid (D6) (211 mg, 0.63 mmol, 1.1 eq.). The reaction mixture was stirred at room temperature for 16 hours before it was diluted with water and extracted with ethyl acetate. The organic layer was washed with brine (4×), dried over sodium sulfate, and concentrated under reduced pressure. The crude residue was purified by flash column chromatography to give 2-(2,6-dioxopiperidin-3-yl)-4-(((1-(1-(1-(5-iodopyridin-2-yl)piperidine-4-carbonyl)piperidin-4-yl)-1H-pyrazol-4-yl)methyl)amino)isoindoline-1,3-dione (D7) (330 mg, 0.42 mmol, 73%), a yellow solid. ¹H NMR (400 MHz, CDCl₃) δ 8.32 (s, 1H), 8.28 (dd, J=2.4, 0.7 Hz, 1H), 7.64 (dd, J=9.0, 2.4 Hz, 1H), 7.54-7.46 (m, 2H), 7.40 (s, 1H), 7.13 (dd, J=7.2, 0.6 Hz, 1H), 6.92 (d, J=8.5 Hz, 1H), 6.51 (dd, J=9.1, 0.8 Hz, 1H), 6.42 (t, J=5.6 Hz, 1H), 4.95-4.86 (m, 1H), 4.73 (d, J=13.6 Hz, 1H), 4.40-4.32 (m, 2H), 4.32-4.21 (m, 2H), 3.23 (t, J=12.9 Hz, 1H), 3.02-2.82 (m, 3H), 2.82-2.66 (m, 4H), 2.24 (d, J=13.0 Hz, 1H), 2.16-2.10 (m, 2H), 2.03-1.67 (m, 8H). LCMS (ESI) C₃₃H₃₅IN₈₀₅ requires 750.17, found 751 (M/Z+H).

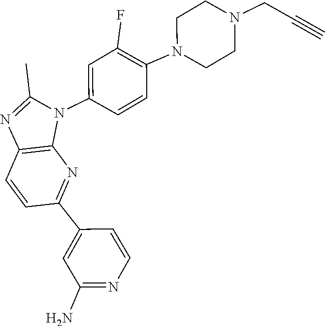 [SMILES: C#CCN1CCN(c2ccc(-n3c(C)nc4ccc(-c5ccnc(N)c5)nc43)cc2F)CC1]

### 4-(3-(3-fluoro-4-(4-(prop-2-yn-1-yl)piperazin-1-yl)phenyl)-2-methyl-3H-imidazo[4,5-b]pyridin-5-yl)pyridin-2-amine (A8)

3-bromoprop-1-yne (9.2 mg, 0.062 mmol, 80% wt, 1.0 eq.) was added to a 5 mL MWV equipped with a magnetic stir bar containing 4-(3-(3-fluoro-4-(piperazin-1-yl)phenyl)-2-methyl-3H-imidazo[4,5-b]pyridine-5-yl)pyridine-2-amine (A7) (25 mg, 0.062 mmol, 1.0 eq.) and potassium carbonate (26 mg, 0.19 mmol, 3.0 eq.) in DMF. The reaction mixture was heated at 90° C. for 16 hours where the reaction mixture was then cooled to room temperature and washed with saturated sodium bicarbonate (5 mL). The mixture was extracted with DCM (3×15 mL) and the organic layers were recombined, dried over sodium sulfate, and concentrated under reduced pressure. The crude residue was purified by automated flash column chromatography using a DCM/MeOH gradient to afford 4-(3-(3-fluoro-4-(4-(prop-2-yn-1-yl)piperazin-1-yl)phenyl)-2-methyl-3H-imidazo[4,5-b]pyridin-5-yl)pyridin-2-amine (A8) (17 mg, 0.039 mmol, 62% yield, eluting at 10% MeOH), a brown solid. ¹H NMR (500 MHz, CDCl₃) δ 8.17 (d, J=5.5 Hz, 1H), 8.12 (d, J=8.3 Hz, 1H), 7.80 (d, J=8.2 Hz, 1H), 7.34-7.15 (m, 5H), 4.83 (s, 2H), 3.49 (d, J=2.5 Hz, 2H), 3.36 (t, J=5.0 Hz, 4H), 2.90 (t, J=4.9 Hz, 4H), 2.67 (s, 3H), 2.40 (t, J=2.4 Hz, 1H). ¹³C NMR (126 MHz, CDCl₃) δ 158.85, 155.30 (d, J=248.7 Hz), 154.65, 149.17, 149.12, 148.96, 147.62, 140.69 (d, J=8.3 Hz), 135.19, 128.17 (d, J=9.7 Hz), 127.07, 123.63 (d, J=3.3 Hz), 119.32 (d, J=4.5 Hz), 116.50, 115.90, 115.72, 112.13, 106.45, 78.63, 73.66, 52.01, 50.32, 50.30, 47.05, 15.44. LCMS (ESI) C₂₅H₂₄FN₇ requires 441.21, found 442 (M/Z+H).

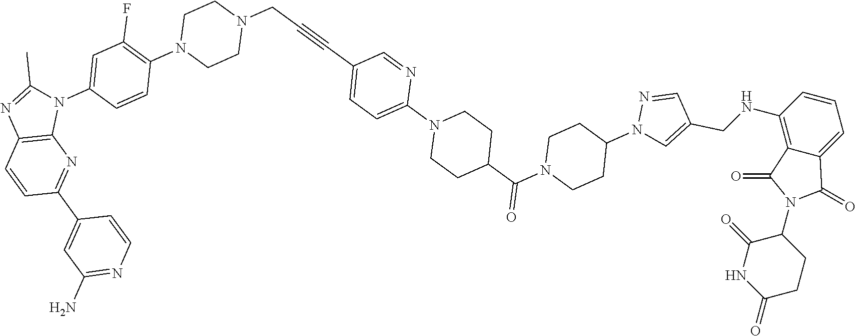 [SMILES: Cc1nc2ccc(-c3ccnc(N)c3)nc2n1-c1ccc(N2CCN(CC#Cc3ccc(N4CCC(C(=O)N5CCC(n6cc(CNc7cccc8c7C(=O)N(C7CCC(=O)NC7=O)C8=O)cn6)CC5)CC4)nc3)CC2)c(F)c1]

### 4-(((1-(1-(1-(5-(3-(4-(4-(5-(2-aminopyridin-4-yl)-2-methyl-3H-imidazo[4,5-b]pyridin-3-yl)-2-fluorophenyl)piperazin-1-yl)prop-1-yn-1-yl)pyridin-2-yl)piperidine-4-carbonyl)piperidin-4-yl)-1H-pyrazol-4-yl)methyl)amino)-2-(2,6-dioxopiperidin-3-yl)isoindoline-1,3-dione (18)

A flame dried 5 ml MWV equipped with a magnetic stir bar containing copper (I) iodide (0.90 mg, 4.7 μmol, 0.25 Eq,), PdCl2 (dppf) (1.9 mg, 2.6 μmol, 0.13 eq.) 4-(3-(3-fluoro-4-(4-(prop-2-yn-1-yl)piperazin-1-yl)phenyl)-2-methyl-3H-imidazo[4,5-b]pyridin-5-yl)pyridin-2-amine (A8) (10 mg, 19 μmol, 1.0 eq.), and 4-(((1-(1-(1-(5-iodopyridin-2-yl)piperidine-4-carbonyl)piperidin-4-yl)-1H-pyrazol-4-yl)methyl)amino)-2-(2,6-dioxopiperidin-3-yl)isoindoline-1,3-dione (D7) (14 mg, 19 μmol, 1.0 eq.) was backfilled with argon 3 times. DMF (0.75 mL) and triethylamine (9.6 mg, 95 μmol, 4.9 eq.) were added sequentially and the reaction mixture was stirred at room temperature for 24 hours. The reaction mixture was concentrated under reduced pressure and purified by automated column chromatography to give 4-(((1-(1-(1-(5-(3-(4-(4-(5-(2-aminopyridin-4-yl)-2-methyl-3H-imidazo[4,5-b]pyridin-3-yl)-2-fluorophenyl)piperazin-1-yl)prop-1-yn-1-yl)pyridin-2-yl)piperidine-4-carbonyl)piperidin-4-yl)-1H-pyrazol-4-yl)methyl)amino)-2-(2,6-dioxopiperidin-3-yl)isoindoline-1,3-dione (18) (12.8 mg, 12.0 μmol, 62%), a yellow solid. ¹H NMR (500 MHz, CDCl₃) δ 8.25 (d, J=2.4 Hz, 1H), 8.13-8.06 (m, 1H), 7.81 (d, J=7.7 Hz, 2H), 7.78-7.72 (m, 1H), 7.54-7.45 (m, 4H), 7.41 (s, 1H), 7.35 (dd, J=6.8, 1.6 Hz, 1H), 7.21-7.14 (m, 3H), 7.11 (d, J=7.1 Hz, 1H), 6.91 (d, J=8.5 Hz, 1H), 6.60 (d, J=9.0 Hz, 1H), 4.89 (dd, J=12.2, 5.4 Hz, 1H), 4.76-4.67 (m, 1H), 4.35 (s, 2H), 4.17-4.03 (m, 1H), 3.91-3.59 (m, 2H), 3.28-3.20 (m, 1H), 3.06-2.91 (m, 2H), 2.91-2.69 (m, 4H), 2.28-2.16 (m, 1H), 2.14-2.08 (m, 1H), 1.90-1.63 (m, 24H). LCMS (ESI) C₅₈H₅₈FN₁₅O₅ requires 1064.2, found 533.15 (M/2).

### Example 19. Synthesis of 4-(((1-(1-(1-(5-(4-(4-(4-(5-(2-aminopyridin-4-yl)-2-methyl-3H-imidazo[4,5-b]pyridin-3-yl)-2- fluorophenyl)piperazin-1-yl) but-1-yn-1-yl)pyridin-2-yl)piperidine-4-carbonyl)piperidin-4-yl)-1H-pyrazol-4-yl)methyl)amino)-2-(2,6-dioxopiperidin-3-yl)isoindoline-1,3-dione (19)

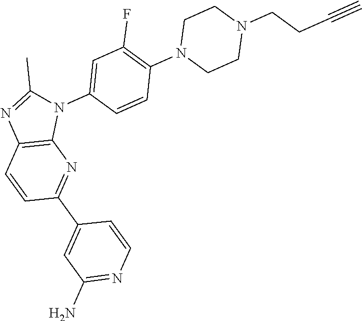 [SMILES: C#CCCN1CCN(c2ccc(-n3c(C)nc4ccc(-c5ccnc(N)c5)nc43)cc2F)CC1]

### 4-(3-(4-(4-(but-3-yn-1-yl)piperazin-1-yl)-3-fluorophenyl)-2-methyl-3H-imidazo[4,5-b]pyridin-5-yl)pyridin-2-amine (A9)

4-bromobut-1-yne (24 mg, 0.18 mmol, 1.1 eq.) was added to a 5 mL MWV equipped with a magnetic stir bar containing 4-(3-(3-fluoro-4-(piperazin-1-yl)phenyl)-2-methyl-3H-imidazo[4,5-b]pyridin-5-yl)pyridin-2-amine (A7) (65 mg, 162 μmol, 1.0 eq.) and potassium carbonate (67 mg, 487 μmol, 3.0 eq.) in DMF (1.60 mL). The reaction mixture was heated at 90° C. for 16 hour where it was then cooled down to room temperature and washed with saturated sodium bicarbonate (5 mL). The mixture was extracted with DCM (3×15 mL) and the organic layers were recombined, dried over sodium sulfate, and concentrated under reduced pressure. The crude residue was purified by flash column chromatography using a DCM/MeOH gradient to afford 4-(3-(4-(4-(but-3-yn-1-yl)piperazin-1-yl)-3-fluorophenyl)-2-methyl-3H-imidazo[4,5-b]pyridin-5-yl)pyridin-2-amine (A9) (28 mg, 61 μmol, 38%), an off-white solid. LCMS (ESI) C₂₆H₂₆FN₇ requires 455.54, found 456.36. (M/Z+H).

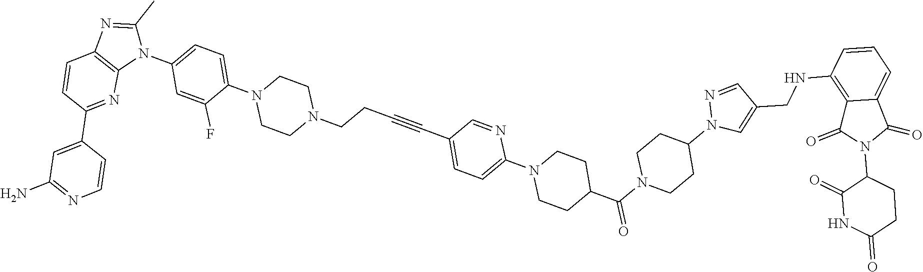 [SMILES: Cc1nc2ccc(-c3ccnc(N)c3)nc2n1-c1ccc(N2CCN(CCC#Cc3ccc(N4CCC(C(=O)N5CCC(n6cc(CNc7cccc8c7C(=O)N(C7CCC(=O)NC7=O)C8=O)cn6)CC5)CC4)nc3)CC2)c(F)c1]

### 4-(((1-(1-(1-(5-(4-(4-(4-(5-(2-aminopyridin-4-yl)-2-methyl-3H-imidazo[4,5-b]pyridin-3-yl)-2-fluorophenyl)piperazin-1-yl) but-1-yn-1-yl)pyridin-2-yl)piperidine-4-carbonyl)piperidin-4-yl)-1H-pyrazol-4-yl)methyl)amino)-2-(2,6-dioxopiperidin-3-yl)isoindoline-1,3-dione (19)

A flame dried 5 ml MWV equipped with a magnetic stir bar containing copper (I) iodide (0.7 mg, 3.6 μmol, 0.12 Eq,), PdCl2 (dppf) (2.6 mg, 3.6 μmol, 0.12 eq.) 4-(3-(4-(4-(but-3-yn-1-yl)piperazin-1-yl)-3-fluorophenyl)-2-methyl-3H-imidazo[4,5-b]pyridin-5-yl)pyridin-2-amine (A9) (13.7 mg, 30.1 μmol, 1.0 eq.), and 4-(((1-(1-(1-(5-iodopyridin-2-yl)piperidine-4-carbonyl)piperidin-4-yl)-1H-pyrazol-4-yl)methyl)amino)-2-(2,6-dioxopiperidin-3-yl)isoindoline-1,3-dione (D7) (22.6 mg, 30.1 μmol, 1.0 eq.) was backfilled with argon 3 times. DMF (0.75 mL) and triethylamine (9.1 mg, 90.2 μmol, 3.0 eq.) were added sequentially and the reaction mixture was stirred at room temperature for 24 hours. The reaction mixture was concentrated under reduced pressure and purified by automated column chromatography to give 4-(((1-(1-(1-(5-(4-(4-(4-(5-(2-aminopyridin-4-yl)-2-methyl-3H-imidazo[4,5-b]pyridin-3-yl)-2-fluorophenyl)piperazin-1-yl) but-1-yn-1-yl)pyridin-2-yl)piperidine-4-carbonyl)piperidin-4-yl)-1H-pyrazol-4-yl)methyl)amino)-2-(2,6-dioxopiperidin-3-yl)isoindoline-1,3-dione (19) (20 mg, 19.0 μmol, 62%), a yellow solid. LCMS (ESI) C₅₉H₆₀FN₁₅O₅ requires 1079.2, found 1080 (M/Z+H).

### Example 20. 4-(((1-(1-(1-(5-(3-((1S,4S)-5-(4-(5-(2-aminopyridin-4-yl)-2-methyl-3H-imidazo[4,5-b]pyridin-3-yl)-2-fluorophenyl)-2,5-diazabicyclo[2.2.1]heptan-2-yl)prop-1-yn-1-yl)pyridin-2-yl)piperidine-4-carbonyl)piperidin-4-yl)-1H-pyrazol-4-yl)methyl)amino)-2-(2,6-dioxopiperidin-3-yl)isoindoline-1,3-dione (20)

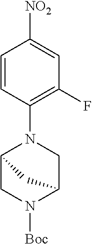 [SMILES: CC(C)(C)OC(=O)N1C[C@@H]2C[C@H]1CN2c1ccc([N+](=O)[O-])cc1F]

### tert-butyl (1S,4S)-5-(2-fluoro-4-nitrophenyl)-2,5-diazabicyclo[2.2.1]heptane-2-carboxylate (20a)

To a 5 mL MWV equipped with a magnetic stir bar containing tert-butyl (1S,4S)-2,5-diazabicyclo[2.2.1]heptane-2-carboxylate (374 mg, 1.89 mmol, 1.0 eq.), potassium carbonate (782 mg, 5.66 mmol, 3.0 eq.), in DMF (2.10 mL) was added 1,2-difluoro-4-nitrobenzene (0.21 mL, 1.89 mmol, 1.0 eq.). The reaction mixture was stirred at 90 C for 18 hours where the resulting mixture was cooled to room temperature and extracted with ethyl acetate (2×20 mL) and washed with water. The organic layer was then washed with water and brine (5×10 mL) to get rid of DMF where the organic layer was then dried over sodium sulfate, filtered off, and concentrated under reduced pressure. The crude residue was purified by flash column chromatography to give tert-butyl (1S,4S)-5-(2-fluoro-4-nitrophenyl)-2,5-diazabicyclo[2.2.1]heptane-2-carboxylate (20a) (555 mg, 1.65 mmol, 87% yield, eluting at 20% ethyl acetate in hexanes), a light-yellow solid. ¹H NMR (500 MHz, CDCl₃) δ 7.91 (dd, J=9.0, 2.6 Hz, 1H), 7.87 (dd, J=14.0, 2.5 Hz, 1H), 6.51 (t, J=8.9 Hz, 1H), 4.72 (s, 1H), 4.67-4.48 (m, 1H), 3.72 (d, J=9.6 Hz, 1H), 3.55-3.24 (m, 3H), 1.96 (s, 2H), 1.42 (s, 9H). ¹³C NMR (126 MHz, CDCl₃) δ 154.17, 149.13 (d, J=242.8 Hz), 141.25 (d, J=9.1 Hz), 137.15 (d, J=7.3 Hz), 121.97, 113.23, 113.11 (d, J=21.4 Hz), 80.29, 60.18, 58.78, 55.76, 53.03, 37.44, 28.57.

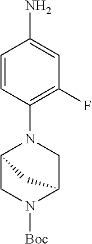 [SMILES: CC(C)(C)OC(=O)N1C[C@@H]2C[C@H]1CN2c1ccc(N)cc1F]

### tert-butyl (1S,4S)-5-(4-amino-2-fluorophenyl)-2,5-diazabicyclo[2.2.1]heptane-2-carboxylate (20b)

Methanol (16.9 mL) was added to a dry 250 mL RBF tert-butyl (1S,4S)-5-(2-fluoro-4-nitrophenyl)-2,5-diazabicyclo[2.2.1]heptane-2-carboxylate (20a) (555 mg, 1.65 mmol, 1.65 mmol) and palladium (55.5 mg, 0.52 mmol, 0.31 mmol). The reaction mixture was stirred under an atmosphere of hydrogen and stirred overnight. After reaction completion (LCMS) the reaction mixture was filtered through celite, concentrated under reduced pressure, and purified by automated column chromatography to give tert-butyl (1S,4S)-5-(4-amino-2-fluorophenyl)-2,5-diazabicyclo[2.2.1]heptane-2-carboxylate (20b) (458 mg, 1.49 mmol, 90.6%). ¹H NMR (500 MHz, CDCl₃) δ 6.60-6.33 (m, 3H), 4.54 (s, 1H), 4.40 (s, 1H), 4.38-4.24 (m, 1H), 4.12-3.68 (bs, 2H), 3.67-3.50, (2H, m), 3.53-3.42 (m, 1H), 3.42-3.27 (m, 1H), 3.27-3.02 (m, 1H), 1.95 (d, J=9.5 Hz, 1H), 1.90-1.80 (m, 1H), 1.43 (d, J=16.3 Hz, 9H).

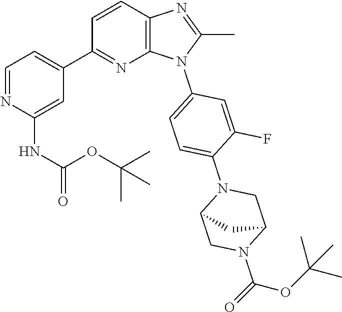 [SMILES: Cc1nc2ccc(-c3ccnc(NC(=O)OC(C)(C)C)c3)nc2n1-c1ccc(N2C[C@@H]3C[C@H]2CN3C(=O)OC(C)(C)C)c(F)c1]

### tert-butyl (1S,4S)-5-(4-(5-(2-((tert-butoxycarbonyl)amino)pyridin-4-yl)-2-methyl-3H-imidazo[4,5-b]pyridin-3-yl)-2-fluorophenyl)-2,5-diazabicyclo[2.2.1]heptane-2-carboxylate (20c)

Potassium phosphate (263 mg, 3 Eq, 1.24 mmol), tert-butyl (5-acetamido-6-chloro-[2,4′-bipyridin]-2′-yl)carbamate (A5) (150 mg, 1 Eq, 413 μmol), tert-butyl 5-(4-amino-2-fluorophenyl)-2,5-diazabicyclo[2.2.1]heptane-2-carboxylate (20b) (140 mg, 1.1 Eq, 455 μmol), dicyclohexyl(2′,4′,6′-triisopropyl-[1,1′-biphenyl]-2-yl)phosphane (19.7 mg, 0.10 Eq, 41.3 μmol), and Pd2(dba)3 (7.57 mg, 0.02 Eq, 8.27 μmol) were added to a flame-dried 5 ml MWV containing a flame dried stir bar. The MWV was capped and purged with argon. Degassed t-BuOH was added to the MWV and heated to 90 C in an oil bath. The reaction mixture was allowed to cool to room temperature and diluted with dichloromethane. The diluted mixture was filtered through Celite with the aid of dichloromethane and 5% MeOH in DCM. The filtrate was concentrated under reduced pressure and purified by automated column chromatography using a CombiFlash to give tert-butyl (1S,4S)-5-(4-(5-(2-((tert-butoxycarbonyl)amino)pyridin-4-yl)-2-methyl-3H-imidazo[4,5-b]pyridin-3-yl)-2-fluorophenyl)-2,5-diazabicyclo[2.2.1]heptane-2-carboxylate (20c) (160 mg, 260 μmol, 62%, eluting at 4% MeOH/DCM) and tert-butyl (4-(3-(4-((1S,4S)-2,5-diazabicyclo[2.2.1]heptan-2-yl)-3-fluorophenyl)-2-methyl-3H-imidazo[4,5-b]pyridin-5-yl)pyridin-2-yl)carbamate (49 mg, 95 μmol, 23%, eluting at 5% MeOH/DCM), a light-brown solid. ¹H NMR (500 MHz, CDCl₃) δ 8.64-8.57 (m, 1H), 8.24 (t, J=5.3 Hz, 2H), 8.09-8.03 (m, 1H), 7.88 (s, 1H), 7.79-7.64 (m, 1H), 7.19-7.06 (m, 2H), 6.75 (t, J=9.1 Hz, 1H), 4.71-4.48 (m, 2H), 3.82-3.70 (m, 1H), 3.70-3.52 (m, 1H), 3.44 (d, J=11.9 Hz, 1H), 3.35 (s, 1H), 2.63-2.59 (m, 3H), 2.06-1.88 (m, 2H), 1.55 (s, 9H), 1.50-1.39 (m, 9H).

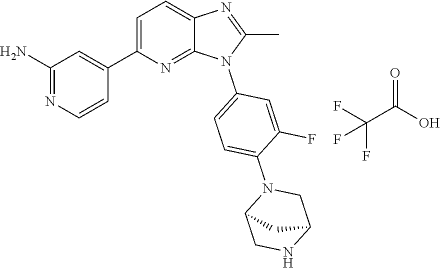

### 4-(3-(4-((1S,4S)-2,5-diazabicyclo[2.2.1]heptan-2-yl)-3-fluorophenyl)-2-methyl-3H-imidazo[4,5-b]pyridin-5-yl)pyridin-2-amine, trifluoroacetic acid (20d)

Trifluoroacetic acid (0.52 mL, 6.79 mmol, 20 eq.) was added to a solution tert-butyl (1S,4S)-5-(4-(5-(2-((tert-butoxycarbonyl)amino)pyridin-4-yl)-2-methyl-3H-imidazo[4,5-b]pyridin-3-yl)-2-fluorophenyl)-2,5-diazabicyclo[2.2.1]heptane-2-carboxylate (20c) (209 mg, 0.33 mmol, 1.0 eq.) in DCM (1.65 mL). The reaction mixture was stirred at room temperature overnight where it was then diluted with DCM and concentrated under reduced pressure (3×). The crude residue was purified by flash column chromatography to give 4-(3-(4-((1S,4S)-2,5-diazabicyclo[2.2.1]heptan-2-yl)-3-fluorophenyl)-2-methyl-3H-imidazo[4,5-b]pyridin-5-yl)pyridin-2-amine, trifluoroacetic acid (20d) (134 mg, 0.33 mmol, 85%), a brown solid. ¹H NMR (400 MHz, DMSO) δ 8.42-8.28 (m, 2H), 8.20 (d, J=8.3 Hz, 1H), 8.02 (dd, J=7.6, 5.3 Hz, 2H), 7.59-7.49 (m, 2H), 7.43 (dd, J=6.8, 1.7 Hz, 1H), 7.33 (dd, J=8.7, 2.2 Hz, 1H), 7.06 (dd, J=9.9, 8.7 Hz, 1H), 4.71 (s, 1H), 4.49 (s, 1H), 3.87-3.78 (m, 1H), 3.56 (dd, J=11.2, 3.9 Hz, 1H), 3.44 (d, J=11.2 Hz, 1H), 3.40-3.32 (m, 1H), 2.52-2.47 (m, 4H), 2.18 (d, J=10.7 Hz, 1H), 2.01 (d, J=10.7 Hz, 1H). ¹³C NMR (101 MHz, DMSO) δ 155.73 (d, J=149.2 Hz), 154.99, 152.11, 151.94, 149.55, 149.15, 144.97, 136.84, 135.89, 135.25 (d, J=9.1 Hz), 135.20, 126.84, 124.36 (d, J=2.7 Hz), 124.03 (d, J=9.8 Hz), 117.15, 116.12, 109.68, 109.00, 57.29, 56.72, 49.50, 48.57, 35.21, 14.91.

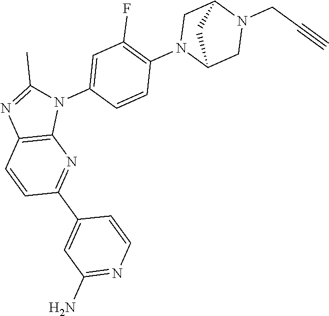 [SMILES: C#CCN1C[C@@H]2C[C@H]1CN2c1ccc(-n2c(C)nc3ccc(-c4ccnc(N)c4)nc32)cc1F]

### 4-(3-(3-fluoro-4-((1S,4S)-5-(prop-2-yn-1-yl)-2,5-diazabicyclo[2.2.1]heptan-2-yl)phenyl)-2-methyl-3H-imidazo[4,5-b]pyridin-5-yl)pyridin-2-amine (20e)

3-bromoprop-1-yne (22 mg, 0.15 mmol, 1.1 eq., 80% Wt.) was added to a 5 mL MWV equipped with a magnetic stir bar containing 4-(3-(4-((1S,4S)-2,5-diazabicyclo[2.2.1]heptan-2-yl)-3-fluorophenyl)-2-methyl-3H-imidazo[4,5-b]pyridin-5-yl)pyridin-2-amine, trifluoroacetic acid (20d) (55 mg, 0.13 mmol, 1.0 eq) and potassium carbonate (55 mg, 0.40 mmol, 3.0 eq.) in DMF (1.3 mL). The reaction mixture was heated at 90° C. for 16 hours it was then cooled down to room temperature and washed with saturated sodium bicarbonate (5 mL). The mixture was extracted with DCM (3×15 mL) and the organic layers were recombined, dried over sodium sulfate, and concentrated under reduced pressure. The crude residue was purified by flash column chromatography using a DCM/MeOH gradient to afford 4-(3-(3-fluoro-4-((1S,4S)-5-(prop-2-yn-1-yl)-2,5-diazabicyclo[2.2.1]heptan-2-yl)phenyl)-2-methyl-3H-imidazo[4,5-b]pyridin-5-yl)pyridin-2-amine (20e) (19 mg, 42 μmol, 32%), a brown solid. LCMS (ESI) C₂₆H₂₄FN₇ requires 453.53, found 454 (M/Z+H).

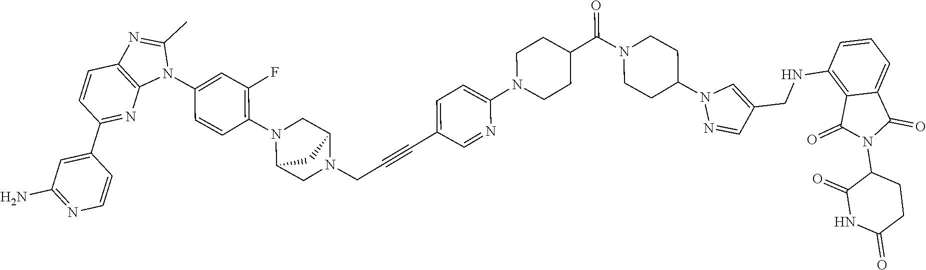 [SMILES: Cc1nc2ccc(-c3ccnc(N)c3)nc2n1-c1ccc(N2C[C@@H]3C[C@H]2CN3CC#Cc2ccc(N3CCC(C(=O)N4CCC(n5cc(CNc6cccc7c6C(=O)N(C6CCC(=O)NC6=O)C7=O)cn5)CC4)CC3)nc2)c(F)c1]

### 4-(((1-(1-(1-(5-(3-((1S,4S)-5-(4-(5-(2-aminopyridin-4-yl)-2-methyl-3H-imidazo[4,5-b]pyridin-3-yl)-2-fluorophenyl)-2,5-diazabicyclo[2.2.1]heptan-2-yl)prop-1-yn-1-yl)pyridin-2-yl)piperidine-4-1,3-dione (20)

A flame dried 5 ml MWV equipped with a magnetic stir bar containing copper (I) iodide (0.4 mg, 2.1 μmol, 0.14 Eq,), PdCl2 (dppf) (1.4 mg, 1.9 μmol, 0.12 eq.) 4-(3-(3-fluoro-4-((1S,4S)-5-(prop-2-yn-1-yl)-2,5-diazabicyclo[2.2.1]heptan-2-yl)phenyl)-2-methyl-3H-imidazo[4,5-b]pyridin-5-yl)pyridin-2-amine (20e) (7.0 mg, 15 μmol, 1.0 eq.), and 4-(((1-(1-(1-(5-iodopyridin-2-yl)piperidine-4-carbonyl)piperidin-4-yl)-1H-pyrazol-4-yl)methyl)amino)-2-(2,6-dioxopiperidin-3-yl)isoindoline-1,3-dione (D7) (12.0 mg, 15 μmol, 1.0 eq.) was backfilled with argon 3 times. DMF (0.75 mL) and triethylamine (4.7 mg, 46 μmol, 3.0 eq.) were added sequentially and the reaction mixture was stirred at room temperature for 7 hours. The reaction mixture was concentrated under reduced pressure and purified by automated column chromatography to give 4-(((1-(1-(1-(5-(3-((1S,4S)-5-(4-(5-(2-aminopyridin-4-yl)-2-methyl-3H-imidazo[4,5-b]pyridin-3-yl)-2-fluorophenyl)-2,5-diazabicyclo[2.2.1]heptan-2-yl)prop-1-yn-1-yl)pyridin-2-yl)piperidine-4-carbonyl)piperidin-4-yl)-1H-pyrazol-4-yl)methyl)amino)-2-(2,6-dioxopiperidin-3-yl)isoindoline-1,3-dione (20) (7.6 mg, 7.1 μmol, 46%), an orange solid. LCMS (ESI) C₅₉H₅₈N₁₅O₅ requires 1075.47, found 1076 (M/Z+H).

### Example 21. 4-(((1-(1-(1-(5-(3-(8-(4-(5-(2-aminopyridin-4-yl)-2-methyl-3H-imidazo[4,5-b]pyridin-3-yl)-2-fluorophenyl)-3,8-diazabicyclo[3.2.1]octan-3-yl)prop-1-yn-1-yl)pyridin-2-yl)piperidine-4-carbonyl)piperidin-4-yl)-1H-pyrazol-4-yl)methyl)amino)-2-(2,6-dioxopiperidin-3-yl)isoindoline-1,3-dione (21)

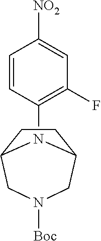 [SMILES: CC(C)(C)OC(=O)N1CC2CCC(C1)N2c1ccc([N+](=O)[O-])cc1F]

### tert-butyl 8-(2-fluoro-4-nitrophenyl)-3,8-diazabicyclo[3.2.1]octane-3-carboxylate (21a)

To a 5 mL MWV equipped with a magnetic stir bar containing 3-Boc-3,8-diazabicyclo[3.2.1]octane (334 mg, 1.57 mmol, 1.0 eq.), potassium carbonate (652 mg, 4.71 mmol, 3.0 eq.), in DMF (1.80 mL) was added 1,2-difluoro-4-nitrobenzene (0.17 mL, 1.57 mmol, 1.0 eq.). The reaction mixture was stirred at 90 C for 18 hours where the resulting mixture was cooled to room temperature and extracted with ethyl acetate (2×20 mL) and washed with water. The organic layer was then washed with water and brine (5×10 mL) to get rid of DMF where the organic layer was then dried over sodium sulfate, filtered off, and concentrated under reduced pressure. The crude residue was purified by flash column chromatography to give tert-butyl 8-(2-fluoro-4-nitrophenyl)-3,8-diazabicyclo[3.2.1]octane-3-carboxylate (21a) (453 mg, 1.29 mmol, 82% yield, eluting at 14% ethyl acetate in hexanes), a yellow solid. ¹H NMR (500 MHz, CDCl₃) δ 7.93 (dd, J=9.2, 2.9 Hz, 1H), 7.89 (dd, J=14.0, 2.6 Hz, 1H), 6.85-6.77 (m, 1H), 4.43 (s, 2H), 3.87 (d, J=13.0 Hz, 1H), 3.73 (d, J=12.9 Hz, 1H), 3.21 (d, J=13.0 Hz, 1H), 3.12 (d, J=13.1 Hz, 1H), 2.06-2.00 (m, 2H), 1.88 (dd, J=17.0, 8.5 Hz, 2H), 1.44 (s, 9H). ¹³C NMR (126 MHz, CDCl₃) δ 155.90, 150.60 (d, J=246.0 Hz), 140.68 (d, J=8.3 Hz), 138.37 (d, J=8.7 Hz), 121.72 (d, J=2.3 Hz), 116.07 (d, J=5.0 Hz), 113.45 (d, J=26.8 Hz), 80.30, 56.43, 48.67, 47.31, 28.48, 26.85, 26.66.

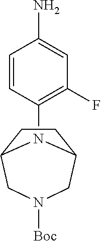 [SMILES: CC(C)(C)OC(=O)N1CC2CCC(C1)N2c1ccc(N)cc1F]

### tert-butyl 8-(4-amino-2-fluorophenyl)-3,8-diazabicyclo[3.2.1]octane-3-carboxylate (21b)

Methanol (12.9 mL) was added to a dry 250 mL RBF containing tert-butyl 8-(2-fluoro-4-nitrophenyl)-3,8-diazabicyclo[3.2.1]octane-3-carboxylate (21a) (453 mg, 1.29 mmol, 1.0 eq.) and palladium (45.3 mg, 0.43 mmol, 0.33 eq.). The reaction mixture was stirred under a atmosphere of hydrogen and stirred overnight. After reaction completion (LCMS) the reaction mixture was filtered through celite, concentrated under reduced pressure, and purified by automated column chromatography to give tert-butyl 8-(4-amino-2-fluorophenyl)-3,8-diazabicyclo[3.2.1]octane-3-carboxylate (21b) (390 mg, 1.21 mmol, 94.1% yield, eluting at 5% MeOH), a dark-brown oil. ¹H NMR (500 MHz, CDCl₃) δ 6.68 (s, 1H), 6.61-6.42 (m, 2H), 5.37-4.14 (bs, 2H), 4.12-3.89 (m, 2H), 3.81 (d, J=12.5 Hz, 1H), 3.68 (d, J=12.5 Hz, 1H), 3.39-3.13 (m, 2H), 1.92 (t, J=6.3 Hz, 2H), 1.87-1.67 (m, 2H).

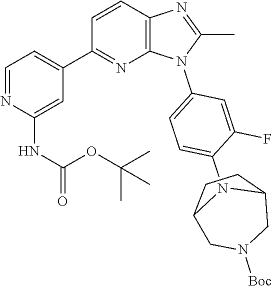 [SMILES: Cc1nc2ccc(-c3ccnc(NC(=O)OC(C)(C)C)c3)nc2n1-c1ccc(N2C3CCC2CN(C(=O)OC(C)(C)C)C3)c(F)c1]

### tert-butyl 8-(4-(5-(2-((tert-butoxycarbonyl)amino)pyridin-4-yl)-2-methyl-3H-imidazo[4,5-b]pyridin-3-yl)-2-fluorophenyl)-3,8-diazabicyclo[3.2.1]octane-3-carboxylate (21c)

Potassium phosphate (263 mg, 3 Eq, 1.24 mmol), tert-butyl (5-acetamido-6-chloro-[2,4′-bipyridin]-2′-yl)carbamate (A5) (150 mg, 1 Eq, 413 μmol), tert-butyl 8-(4-amino-2-fluorophenyl)-3,8-diazabicyclo[3.2.1]octane-3-carboxylate (21b) (146 mg, 1.1 Eq, 455 μmol), dicyclohexyl(2′,4′,6′-triisopropyl-[1,1′-biphenyl]-2-yl)phosphane (19.7 mg, 0.10 Eq, 41.3 μmol), and Pd2(dba)3 (7.57 mg, 0.02 Eq, 8.27 μmol) were added to a flame-dried 5 ml MWV containing a flame dried stir bar. The MWV was capped and purged with argon. Degassed t-BuOH was added to the MWV and heated to 90 C in an oil bath. The reaction mixture was allowed to cool to room temperature and diluted with dichloromethane. The diluted mixture was filtered through Celite with the aid of dichloromethane and 5% MeOH in DCM. The filtrate was concentrated under reduced pressure and purified by automated column chromatography using a CombiFlash to tert-butyl 8-(4-(5-(2-((tert-butoxycarbonyl)amino)pyridin-4-yl)-2-methyl-3H-imidazo[4,5-b]pyridin-3-yl)-2-fluorophenyl)-3,8-diazabicyclo[3.2.1]octane-3-carboxylate (21c) (95 mg, 0.15 mmol, 36%, eluting at 4% MeOH/DCM), a brown solid ¹H NMR (500 MHz, CDCl₃) δ 8.64-8.56 (m, 1H), 8.38-8.16 (m, 2H), 8.10-8.01 (m, 1H), 7.92-7.84 (m, 1H), 7.66 (ddt, J=7.2, 4.9, 2.9 Hz, 1H), 7.20 (dd, J=13.4, 2.4 Hz, 1H), 7.15 (dd, J=8.7, 2.4 Hz, 1H), 7.02 (t, J=9.0 Hz, 1H), 4.27 (d, J=14.1 Hz, 2H), 3.89 (d, J=12.9 Hz, 1H), 3.74 (d, J=12.7 Hz, 1H), 3.35 (d, J=12.7 Hz, 1H), 3.31-3.22 (m, 1H), 2.61 (s, 3H), 2.09-1.99 (m, 2H), 1.92-1.82 (m, 2H), 1.54 (s, 9H), 1.48 (s, 9H).

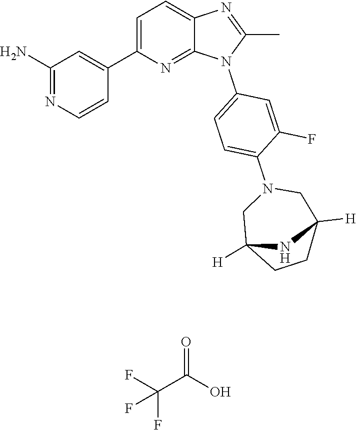 [SMILES: Cc1nc2ccc(-c3ccnc(N)c3)nc2n1-c1ccc(N2C[C@H]3CC[C@@H](C2)N3)c(F)c1 ; O=C(O)C(F)(F)F]

### 4-(3-(4-((1R,5S)-3,8-diazabicyclo[3.2.1]octan-3-yl)-3-fluorophenyl)-2-methyl-3H-imidazo[4,5-b]pyridin-5-yl)pyridin-2-amine, trifluoroacetic acid (21d)

Trifluoroacetic acid (0.38 mL, 4.92 mmol, 20 eq.) was added to a 5 mL MWV equipped with a magnetic stir bar containing tert-butyl 8-(4-(5-(2-((tert-butoxycarbonyl)amino)pyridin-4-yl)-2-methyl-3H-imidazo[4,5-b]pyridin-3-yl)-2-fluorophenyl)-3,8-diazabicyclo[3.2.1]octane-3-carboxylate (21c) (155 mg, 0.24 mmol, 1.0 eq.) in DCM (1.2 mL). The reaction mixture was stirred overnight at room temperature where it was then diluted with DCM and concentrated under reduced pressure (3×). The crude residue was purified by flash column chromatography to give 4-(3-(4-((1R,5S)-3,8-diazabicyclo[3.2.1]octan-3-yl)-3-fluorophenyl)-2-methyl-3H-imidazo[4,5-b]pyridin-5-yl)pyridin-2-amine, trifluoroacetic acid (21d) (82 mg, 0.24 mmol, 61%, eluting at 20% MeOH in DCM), a brown solid. ¹H NMR (400 MHz, DMSO) δ 8.22 (d, J=8.3 Hz, 1H), 8.03 (s, 1H), 8.01 (d, J=2.2 Hz, 1H), 7.59 (dd, J=13.7, 2.4 Hz, 1H), 7.52 (s, 1H), 7.43 (dd, J=6.7, 1.7 Hz, 1H), 7.39 (dd, J=8.7, 2.3 Hz, 1H), 7.30 (t, J=9.2 Hz, 1H), 4.43 (s, 2H), 3.32-3.19 (m, 4H), 2.52 (s, 3H), 2.17-2.04 (m, 5H). ¹³C NMR (101 MHz, DMSO) δ 156.75, 155.70, 154.18, 152.7, 152.24, 145.67, 138.0, 136.35, 135.6, 127.38, 124.92, 118.89, 117.64, 116.88, 110.19, 109.21, 99.33, 55.30, 46.95, 25.87, 15.49.

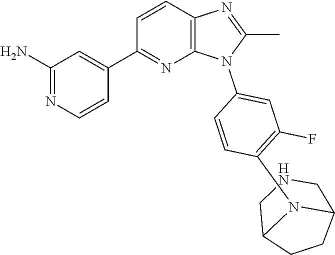 [SMILES: Cc1nc2ccc(-c3ccnc(N)c3)nc2n1-c1ccc(N2C3CCC2CNC3)c(F)c1]

### 4-(3-(4-(3,8-diazabicyclo[3.2.1]octan-8-yl)-3-fluorophenyl)-2-methyl-3H-imidazo[4,5-b]pyridin-5-yl)pyridin-2-amine (21e)

3-bromoprop-1-yne (25 mg, 0.17 mmol, 1.1 eq., 80% Wt.) was added to a 5 mL MWV equipped with a magnetic stir bar tert-butyl 8-(4-(5-(2-((tert-butoxycarbonyl)amino)pyridin-4-yl)-2-methyl-3H-imidazo[4,5-b]pyridin-3-yl)-2-fluorophenyl)-3,8-diazabicyclo[3.2.1]octane-3-carboxylate (21d) (65.7 mg, 153 μmol, 1.0 eq.) and potassium carbonate (63.4 mg, 459 μmol, 3.0 eq.) in DMF (1.55 mL). The reaction mixture was heated at 90° C. for 16 hours where it was then cooled down to room temperature and washed with saturated sodium bicarbonate (5 mL). The mixture was extracted with DCM (3×15 mL) and the organic layers were recombined, dried over sodium sulfate, and concentrated under reduced pressure. The crude residue was purified by flash column chromatography using a DCM/MeOH gradient to afford 4-(3-(4-(3,8-diazabicyclo[3.2.1]octan-8-yl)-3-fluorophenyl)-2-methyl-3H-imidazo[4,5-b]pyridin-5-yl)pyridin-2-amine (39 mg, 84 μmol, 55%, eluting at 40% MeOH in DCM), a brown solid. LCMS (ESI) C₂₇H₂₆FN₇ requires 467.55, found 468 ((M/Z)+H).

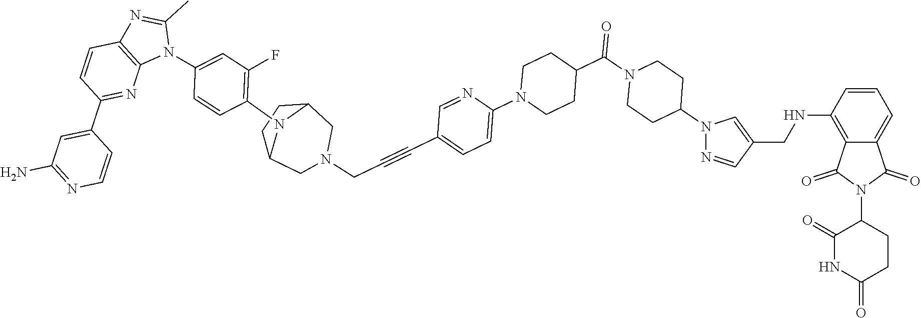 [SMILES: Cc1nc2ccc(-c3ccnc(N)c3)nc2n1-c1ccc(N2C3CCC2CN(CC#Cc2ccc(N4CCC(C(=O)N5CCC(n6cc(CNc7cccc8c7C(=O)N(C7CCC(=O)NC7=O)C8=O)cn6)CC5)CC4)nc2)C3)c(F)c1]

### 4-(((1-(1-(1-(5-(3-(8-(4-(5-(2-aminopyridin-4-yl)-2-methyl-3H-imidazo[4,5-b]pyridin-3-yl)-2-fluorophenyl)-3,8-diazabicyclo[3.2.1]octan-3-yl)prop-1-yn-1-yl)pyridin-2-yl)piperidine-4-carbonyl)piperidin-4-yl)-1H-pyrazol-4-yl)methyl)amino)-2-(2,6-dioxopiperidin-3-yl)isoindoline-1,3-dione (21)

A flame dried 5 ml MWV equipped with a magnetic stir bar containing copper (I) iodide (0.9 mg, 4.9 μmol, 0.12 Eq,), PdCl2 (dppf) (3.6 mg, 4.9 μmol, 0.12 eq.) 4-(3-(4-(3,8-diazabicyclo[3.2.1]octan-8-yl)-3-fluorophenyl)-2-methyl-3H-imidazo[4,5-b]pyridin-5-yl)pyridin-2-amine (21e) (19 mg, 41 μmol, 1.0 eq.), and 4-(((1-(1-(1-(5-iodopyridin-2-yl)piperidine-4-carbonyl)piperidin-4-yl)-1H-pyrazol-4-yl)methyl)amino)-2-(2,6-dioxopiperidin-3-yl)isoindoline-1,3-dione (D7) (31 mg, 41 μmol, 1.0 eq.) was backfilled with argon 3 times. DMF (0.75 mL) and triethylamine (12 mg, 0.12 mmol, 3.0 eq.) were added sequentially and the reaction mixture was stirred at room temperature for 24 hours. The reaction mixture was concentrated under reduced pressure and purified by automated column chromatography to give 4-(((1-(1-(1-(5-(3-(8-(4-(5-(2-aminopyridin-4-yl)-2-methyl-3H-imidazo[4,5-b]pyridin-3-yl)-2-fluorophenyl)-3,8-diazabicyclo[3.2.1]octan-3-yl)prop-1-yn-1-yl)pyridin-2-yl)piperidine-4-carbonyl)piperidin-4-yl)-1H-pyrazol-4-yl)methyl)amino)-2-(2,6-dioxopiperidin-3-yl)isoindoline-1,3-dione (21) (25 mg, 23 μmol, 56%), a yellow solid. LCMS (ESI) C₆₀H₆₀FN₁₅O₅ requires 1089.48, found 1090 (M/2).

### Example 22. Synthesis of 4-(((1-(1-(1-(5-(3-((3aR,6aS)-5-(4-(5-(2-aminopyridin-4-yl)-2-methyl-3H-imidazo[4,5-b]pyridin-3-yl)-2-fluorophenyl) hexahydropyrrolo[3,4-c]pyrrol-2 (1H)-yl)prop-1-yn-1-yl)pyridin-2-yl)piperidine-4-carbonyl)piperidin-4-yl)-1H-pyrazol-4-yl)methyl)amino)-2-(2,6-dioxopiperidin-3-yl)isoindoline-1,3-dione (22)

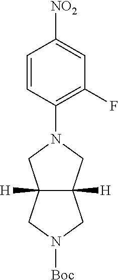 [SMILES: CC(C)(C)OC(=O)N1C[C@@H]2CN(c3ccc([N+](=O)[O-])cc3F)C[C@@H]2C1]

### tert-butyl (3aR,6aS)-5-(2-fluoro-4-nitrophenyl) hexahydropyrrolo[3,4-c]pyrrole-2 (1H)-carboxylate (22a)

To a 5 mL MWV equipped with a magnetic stir bar containing tert-butyl (3aR,6aS)-hexahydropyrrolo[3,4-c]pyrrole-2 (1H)-carboxylate (400 mg, 1.89 mmol, 1.0 eq.), potassium carbonate (782 mg, 5.66 mmol, 3.0 eq.), in DMF (2.10 mL) was added 1,2-difluoro-4-nitrobenzene (0.21 mL, 1.89 mmol, 1.0 eq.). The reaction mixture was stirred at 90° C. for 18 hours where the resulting mixture was cooled to room temperature and extracted with ethyl acetate (2×20 mL) and washed with water. The organic layer was then washed with water and brine (5×10 mL) to get rid of DMF where the organic layer was then dried over sodium sulfate, filtered off, and concentrated under reduced pressure. The crude residue was purified by automated column chromatography to give tert-butyl (3aR,6aS)-5-(2-fluoro-4-nitrophenyl) hexahydropyrrolo[3,4-c]pyrrole-2 (1H)-carboxylate (22a) (568 mg, 1.89 mmol, 85% yield, eluting at 40% ethyl acetate in hexanes), a yellow solid. ¹H NMR (500 MHz, CDCl₃) δ 7.92 (dt, J=9.3, 2.7 Hz, 1H), 7.87 (dt, J=14.1, 2.8 Hz, 1H), 6.53 (td, J=8.9, 1.9 Hz, 1H), 3.82 (td, J=6.2, 2.6 Hz, 2H), 3.70-3.62 (m, 2H), 3.50 (dt, J=11.0, 3.4 Hz, 2H), 3.32 (dd, J=11.9, 3.7 Hz, 2H), 3.04-2.96 (m, 2H), 1.46 (d, J=1.6 Hz, 9H). ¹³C NMR (126 MHz, CDCl₃) δ 154.58, 149.24 (d, J=244.0 Hz), 142.01 (d, J=8.9 Hz), 121.90 (d, J=1.9 Hz), 113.53 (d, J=5.8 Hz), 112.94 (d, J=26.4 Hz), 79.88, 54.03 (d, J=6.0 Hz), 49.92, 41.55, 28.60.

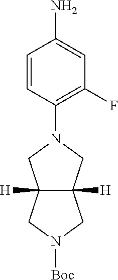 [SMILES: CC(C)(C)OC(=O)N1C[C@@H]2CN(c3ccc(N)cc3F)C[C@@H]2C1]

### tert-butyl (3aR,6aS)-5-(4-amino-2-fluorophenyl) hexahydropyrrolo[3,4-c]pyrrole-2 (1H)-carboxylate (22b)

Methanol (16.2 mL) was added to a dry 250 mL RBF containing tert-butyl (3aR,6aS)-5-(2-fluoro-4-nitrophenyl) hexahydropyrrolo[3,4-c]pyrrole-2 (1H)-carboxylate (22a) (568 mg, 1.62 mmol, 1.0 eq.) and palladium on carbon (57 mg, 0.54 mmol, 0.33 eq.). The reaction mixture was stirred under an atmosphere of hydrogen and stirred overnight. After reaction completion (TLC), the reaction mixture was filtered through celite and washed with ethyl acetate and methanol (1:1). The filtrate was concentrated under reduced pressure and purified by automated flash column chromatography to give tert-butyl (3aR,6aS)-5-(4-amino-2-fluorophenyl) hexahydropyrrolo[3,4-c]pyrrole-2 (1H)-carboxylate (22b) (502 mg, 1.56 mmol, 96% yield, eluting at 2% MeOH), a dark-brown oil. ¹H NMR (500 MHz, CDCl₃) δ 6.69 (s, 1H), 6.62-6.42 (m, 2H), 5.21-4.21 (bs, 2H) 3.62 (s, 2H), 3.44 (s, 2H), 3.40-3.19 (m, 2H), 3.14 (s, 2H), 2.96 (s, 2H), 1.46 (s, 9H).

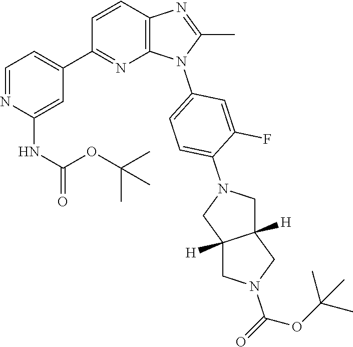 [SMILES: Cc1nc2ccc(-c3ccnc(NC(=O)OC(C)(C)C)c3)nc2n1-c1ccc(N2C[C@H]3CN(C(=O)OC(C)(C)C)C[C@H]3C2)c(F)c1]

### tert-butyl (3aR,6aS)-5-(4-(5-(2-((tert-butoxycarbonyl)amino)pyridin-4-yl)-2-methyl-3H-imidazo[4,5-b]pyridin-3-yl)-2-fluorophenyl) hexahydropyrrolo[3,4-c]pyrrole-2 (1H)-carboxylate (22c)

potassium phosphate (263 mg, 3 Eq, 1.24 mmol), tert-butyl (5-acetamido-6-chloro-[2,4′-bipyridin]-2′-yl)carbamate (A5) (150 mg, 1 Eq, 413 μmol), tert-butyl (3aR,6aS)-5-(4-amino-2-fluorophenyl) hexahydropyrrolo[3,4-c]pyrrole-2 (1H)-carboxylate (22b) (146 mg, 1.1 Eq, 455 μmol), dicyclohexyl(2′,4′,6′-triisopropyl-[1,1′-biphenyl]-2-yl)phosphane (19.7 mg, 0.10 Eq, 41.3 μmol), and Pd2(dba)3 (7.57 mg, 0.02 Eq, 8.27 μmol) were added to a flame-dried 5 ml MWV containing a flame dried stir bar. The MWV was capped and purged with argon. Degassed t-BuOH was added to the MWV and heated to 90° C. in an oil bath. The reaction mixture was allowed to cool to room temperature and diluted with dichloromethane. The diluted mixture was filtered through Celite with the aid of dichloromethane and 5% MeOH in DCM. The filtrate was concentrated under reduced pressure and purified by flash column chromatography to give tert-butyl (3aR,6aS)-5-(4-(5-(2-((tert-butoxycarbonyl)amino)pyridin-4-yl)-2-methyl-3H-imidazo[4,5-b]pyridin-3-yl)-2-fluorophenyl) hexahydropyrrolo[3,4-c]pyrrole-2 (1H)-carboxylate (22c) (162 mg, 257 μmol, 62.2%). ¹H NMR (500 MHz, CDCl₃) δ 8.89-8.72 (m, 1H), 8.18-8.07 (m, 2H), 7.93 (t, J=8.1 Hz, 1H), 7.86-7.75 (m, 1H), 7.57-7.35 (m, 1H), 7.17-7.07 (m, 2H), 6.80 (t, J=9.0 Hz, 1H), 3.75-3.62 (m, 4H), 3.45 (d, J=10.4 Hz, 2H), 3.40-3.23 (m, 2H) 3.01 (s, 2H), 2.62 (d, J=3.3 Hz, 3H), 1.55 (s, 9H), 1.48 (s, 9H).

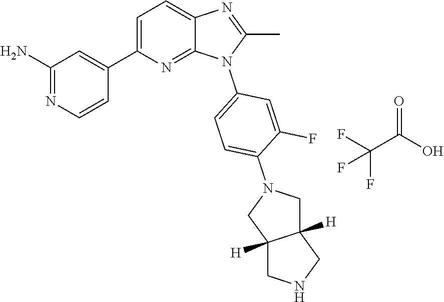

### 4-(3-(3-fluoro-4-((3aR,6aS)-hexahydropyrrolo[3,4-c]pyrrol-2 (1H)-yl)phenyl)-2-methyl-3H-imidazo[4,5-b]pyridin-5-yl)pyridin-2-amine, trifluoroacetic acid (22d)

Trifluoroacetic acid (0.40 mL, 5.14 mmol, 20 eq.) was added dropwise to a 5 mL MWV equipped with a magnetic stir bar containing tert-butyl (3aR,6aS)-5-(4-(5-(2-((tert-butoxycarbonyl)amino)pyridin-4-yl)-2-methyl-3H-imidazo[4,5-b]pyridin-3-yl)-2-fluorophenyl) hexahydropyrrolo[3,4-c]pyrrole-2 (1H)-carboxylate (22c) (162 mg, 0.25 mmol, 1.0 eq.) in DCM (1.3 mL). The reaction mixture was stirred at room temperature overnight where it was then diluted with DCM and concentrated under reduced pressure (3×). The crude residue was purified by flash column chromatography to give 4-(3-(3-fluoro-4-((3aR,6aS)-hexahydropyrrolo[3,4-c]pyrrol-2 (1H)-yl)phenyl)-2-methyl-3H-imidazo[4,5-b]pyridin-5-yl)pyridin-2-amine, trifluoroacetic acid (22d) (127 mg, 0.25 mmol, 90%, eluting at 18% MeOH, in DCM), a yellow solid. ¹H NMR (400 MHz, DMSO) δ 9.24 (s, 2H), 8.20 (d, J=8.3 Hz, 1H), 8.08 (s, 2H), 8.04-7.96 (m, 2H), 7.57-7.47 (m, 2H), 7.41 (dd, J=6.7, 1.7 Hz, 1H), 7.38-7.30 (m, 1H), 7.03 (dd, J=9.8, 8.7 Hz, 1H), 3.59-3.47 (m, 4H), 3.42 (dd, J=9.8, 5.5 Hz, 2H), 3.10 (h, J=4.3 Hz, 4H), 2.50 (s, 3H).

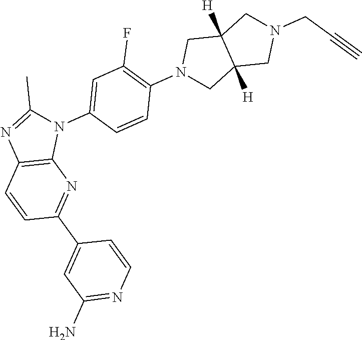 [SMILES: C#CCN1C[C@@H]2CN(c3ccc(-n4c(C)nc5ccc(-c6ccnc(N)c6)nc54)cc3F)C[C@@H]2C1]

### 4-(3-(3-fluoro-4-((3aR,6aS)-5-(prop-2-yn-1-yl) hexahydropyrrolo[3,4-c]pyrrol-2 (1H)-yl)phenyl)-2-methyl-3H-imidazo[4,5-b]pyridin-5-yl)pyridin-2-amine (22e)

3-bromoprop-1-yne (24 mg, 0.16 mmol, 1.0 eq., 80% Wt.) was added to a 5 mL MWV equipped with a magnetic stir bar containing 4-(3-(3-fluoro-4-(hexahydropyrrolo[3,4-c]pyrrol-2 (1H)-yl)phenyl)-2-methyl-3H-imidazo[4,5-b]pyridin-5-yl)pyridin-2-amine (22d) (70 mg, 0.16 mmol, 1.0 eq.) and potassium carbonate (68 mg, 0.49 mmol, 3.0 eq.) in DMF (1.60 mL). The reaction mixture was heated at 90° C. for 16 hours where it was then cooled down to room temperature and washed with saturated sodium bicarbonate (5 mL). The mixture was extracted with DCM (3×15 mL) and the organic layers were recombined, dried over sodium sulfate, and concentrated under reduced pressure. The crude residue was purified by automated flash column chromatography using a DCM/MeOH gradient to afford 4-(3-(3-fluoro-4-(5-(prop-2-yn-1-yl) hexahydropyrrolo[3,4-c]pyrrol-2 (1H)-yl)phenyl)-2-methyl-3H-imidazo[4,5-b]pyridin-5-yl)pyridin-2-amine (22c) (31 mg, 66 μmol, 41%), a brown solid. LCMS (ESI) C₂₇H₂₆FN₇ requires 467.55, found 468 (M/Z+H).

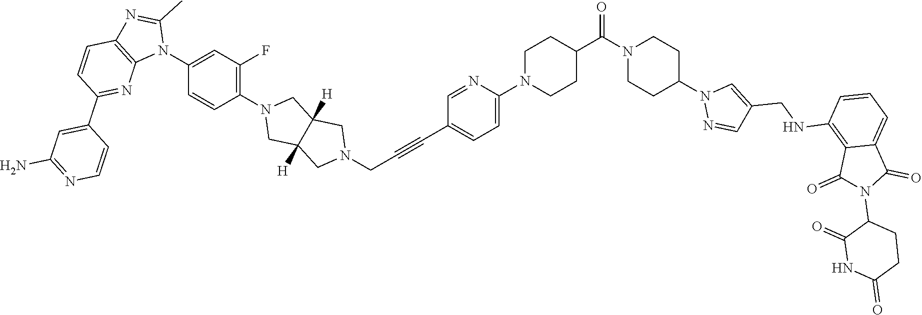 [SMILES: Cc1nc2ccc(-c3ccnc(N)c3)nc2n1-c1ccc(N2C[C@H]3CN(CC#Cc4ccc(N5CCC(C(=O)N6CCC(n7cc(CNc8cccc9c8C(=O)N(C8CCC(=O)NC8=O)C9=O)cn7)CC6)CC5)nc4)C[C@H]3C2)c(F)c1]

### 4-(((1-(1-(1-(5-(3-((3aR,6aS)-5-(4-(5-(2-aminopyridin-4-yl)-2-methyl-3H-imidazo[4,5-b]pyridin-3-yl)-2-fluorophenyl) hexahydropyrrolo[3,4-c]pyrrol-2 (1H)-yl)prop-1-yn-1-yl)pyridin-2-yl)piperidine-4-carbonyl)piperidin-4-yl)-1H-pyrazol-4-yl)methyl)amino)-2-(2,6-dioxopiperidin-3-yl)isoindoline-1,3-dione (22)

A flame dried 5 ml MWV equipped with a magnetic stir bar containing copper (I) iodide (0.41 mg, 2.1 μmol, 0.20 Eq,), PdCl2 (dppf) (0.9 mg, 1.3 μmol, 0.12 eq.) 4-(3-(3-fluoro-4-((3aR,6aS)-5-(prop-2-yn-1-yl) hexahydropyrrolo[3,4-c]pyrrol-2 (1H)-yl)phenyl)-2-methyl-3H-imidazo[4,5-b]pyridin-5-yl)pyridin-2-amine (22e) (5.0 mg, 11 μmol, 1.0 eq.), and 4-(((1-(1-(1-(5-iodopyridin-2-yl)piperidine-4-carbonyl)piperidin-4-yl)-1H-pyrazol-4-yl)methyl)amino)-2-(2,6-dioxopiperidin-3-yl)isoindoline-1,3-dione (D7) (8.0 mg, 11 μmol, 1.0 eq.) was backfilled with argon 3 times. DMF (0.75 mL) and triethylamine (3.2 mg, 32 μmol, 3.0 eq.) were added sequentially and the reaction mixture was stirred at room temperature for 24 hours. The reaction mixture was concentrated under reduced pressure and purified by automated column chromatography to give 4-(((1-(1-(1-(5-(3-((3aR,6aS)-5-(4-(5-(2-aminopyridin-4-yl)-2-methyl-3H-imidazo[4,5-b]pyridin-3-yl)-2-fluorophenyl) hexahydropyrrolo[3,4-c]pyrrol-2 (1H)-yl)prop-1-yn-1-yl)pyridin-2-yl)piperidine-4-carbonyl)piperidin-4-yl)-1H-pyrazol-4-yl)methyl)amino)-2-(2,6-dioxopiperidin-3-yl)isoindoline-1,3-dione (22) (3.0 mg, 2.8 μmol, 26%), a yellow solid. ¹H NMR (500 MHz, CDCl₃) δ 8.31-8.17 (m, 2H), 8.10 (d, J=8.1 Hz, 1H), 7.82 (d, J=8.4 Hz, 1H), 7.76 (d, J=6.9 Hz, 1H), 7.54-7.46 (m, 2H), 7.46-7.38 (m, 2H), 7.17-7.03 (m, 4H), 6.93 (d, J=8.6 Hz, 1H), 6.80 (t, J=8.9 Hz, 1H), 6.75-6.57 (m, 2H), 6.43 (s, 1H), 5.12-5.07 (m, 1H), 4.94-4.86 (m, 1H), 4.76-4.62 (m, 2H), 4.55-4.44 (m, 1H), 4.43-4.23 (m, 5H), 3.39 (dp, J=2.9, 1.6 Hz, 3H), 3.31-3.20 (m, 2H), 3.09-2.91 (m, 3H), 2.90-2.68 (m, 5H), 2.62-2.57 (m, 2H), 2.19-2.08 (m, 2H), 2.08-2.01 (m, 1H), 1.83 (m, 5H). ¹⁹F NMR (471 MHz, CDCl₃) δ -124.67. LCMS (ESI) C₆₀H₆₀FN₁₅O₅ requires 1089.48, found 1090 (M/Z+H).

### Example 23. Synthesis of 4-(((1-(1-(1-(5-(3-(4-(4-(5-(2-aminopyridin-4-yl)-2-methyl-3H-imidazo[4,5-b]pyridin-3-yl)-2,6-difluorophenyl)piperazin-1-yl)prop-1-yn-1-yl)pyridin-2-yl)piperidine-4-carbonyl)piperidin-4-yl)-1H-pyrazol-4-yl)methyl)amino)-2-(2,6-dioxopiperidin-3-yl)isoindoline-1,3-dione (23)

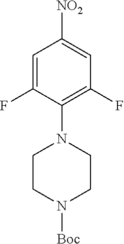 [SMILES: CC(C)(C)OC(=O)N1CCN(c2c(F)cc([N+](=O)[O-])cc2F)CC1]

### tert-butyl 4-(2,6-difluoro-4-nitrophenyl)piperazine-1-carboxylate (23a)

To a 5 mL MWV equipped with a magnetic stir bar containing tert-butyl piperazine-1-carboxylate (1.05 g, 5.64 mmol, 1.0 eq.), potassium carbonate (2.34 g, 16.94 mmol, 3.0 eq.), in DMF (5.65 mL) was added 1,2,3-trifluoro-5-nitrobenzene (0.66 mL, 5.64 mmol, 1.0 eq.). The reaction mixture was stirred at 90° C. for 18 hours where the resulting mixture was cooled to room temperature and extracted with ethyl acetate (2×20 mL) and washed with water. The organic layer was then washed with water and brine (5×10 mL) to get rid of DMF where the organic layer was then dried over sodium sulfate, filtered off, and concentrated under reduced pressure and purified by flash column chromatography to give tert-butyl 4-(2,6-difluoro-4-nitrophenyl)piperazine-1-carboxylate (23a) (1.22 g, 3.57 mmol, 63%), an orange solid. ¹H NMR (400 MHz, CDCl₃) δ 7.83-7.71 (m, 2H), 3.59-3.52 (m, 4H), 3.31 (tt, J=4.9, 1.6 Hz, 4H), 1.48 (s, 9H). ¹³C NMR (101 MHz, CDCl₃) δ 156.63 (d, J=7.9 Hz), 154.79, 154.15 (d, J=7.9 Hz), 134.63, 109.24 (d, J=10.6 Hz), 109.04 (d, J=10.2 Hz), 80.33, 50.67 (t, J=4.1 Hz), 44.28, 28.55. LCMS (ESI) C₁₅H₁₉F2N₃₀₄ requires 343.13, found 288 (M/Z+H), carbamic acid fragment.

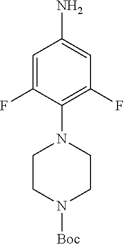 [SMILES: CC(C)(C)OC(=O)N1CCN(c2c(F)cc(N)cc2F)CC1]

### tert-butyl 4-(4-amino-2,6-difluorophenyl)piperazine-1-carboxylate (23b)

To a 20 mL MW vial equipped with a magnetic stir bar containing tert-butyl 4-(2,6-difluoro-4-nitrophenyl)piperazine-1-carboxylate (23a) (700 mg, 2.04 mmol, 1.0 eq.) in Ethanol (14.3 mL) and Water (3.6 mL) was added ammonium chloride (65 mg, 1.22 mmol, 0.6 eq.) and was stirred at 40° C. Iron (683 mg, 12.2 mmol, 6.0 eq.) was then added and heated to 90° C. and stirred for 2 hours. After cooling to room temperature, the reaction mixture was filtered and partitioned between DCM and a saturated solution of sodium bicarbonate. The aqueous phase was extracted with DCM (3 x) and the combined organic phases were dried over sodium sulfate. The residue was redissolved in chloroform and concentrated under reduced pressure (3 x) to give tert-butyl 4-(4-amino-2,6-difluorophenyl)piperazine-1-carboxylate (23b) (641 mg, 2.05 mmol, 100%). LCMS (ESI) C₁₅H₂₁F₂N₃₀₂ requires 313.16, found 314 (M/Z+H).

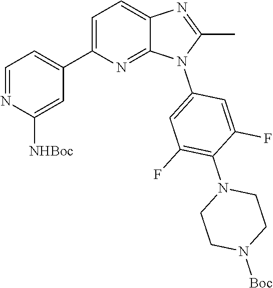 [SMILES: Cc1nc2ccc(-c3ccnc(NC(=O)OC(C)(C)C)c3)nc2n1-c1cc(F)c(N2CCN(C(=O)OC(C)(C)C)CC2)c(F)c1]

### tert-butyl 4-(4-(5-(2-((tert-butoxycarbonyl)amino)pyridin-4-yl)-2-methyl-3H-imidazo[4,5-b]pyridin-3-yl)-2,6-difluorophenyl)piperazine-1-carboxylate (23c)

potassium phosphate (369 mg, 3 Eq, 1.74 mmol), tert-butyl (5-acetamido-6-chloro-[2,4′-bipyridin]-2′-yl)carbamate (A5) (210 mg, 1 Eq, 579 μmol), tert-butyl 4-(4-amino-2,6-difluorophenyl)piperazine-1-carboxylate (23b) (200 mg, 1.1 Eq, 637 μmol), dicyclohexyl(2′,4′,6′-triisopropyl-[1,1′-biphenyl]-2-yl)phosphane (27.6 mg, 0.10 Eq, 57.9 μmol), and Pd2(dba)3 (10.6 mg, 0.02 Eq, 11.6 μmol) were added to a flame-dried 5 ml MWV containing a flame dried stir bar. The MWV was capped and purged with argon. Degassed t-BuOH was added to the MWV and heated to 90 C in an oil bath. The reaction mixture was allowed to cool to room temperature and diluted with dichloromethane. The diluted mixture was filtered through Celite with the aid of dichloromethane and 5% MeOH in DCM. The filtrate was concentrated under reduced pressure and purified by flash column chromatography to give tert-butyl 4-(4-(5-(2-((tert-butoxycarbonyl)amino)pyridin-4-yl)-2-methyl-3H-imidazo[4,5-b]pyridin-3-yl)-2,6-difluorophenyl)piperazine-1-carboxylate (23c) (282 mg, 454 μmol, 78.4%), a white solid. ¹H NMR (400 MHz, CDCl₃) δ 8.58-8.48 (m, 1H), 8.27-8.19 (m, 1H), 8.10-8.02 (m, 1H), 7.95-7.86 (m, 1H), 7.68-7.62 (m, 1H), 7.41-7.33 (m, 1H), 7.15-7.06 (m, 2H), 3.62-3.52 (m, 4H), 3.31-3.15 (m, 4H), 2.66-2.60 (m, 3H), 1.60-1.37 (m, 18H).

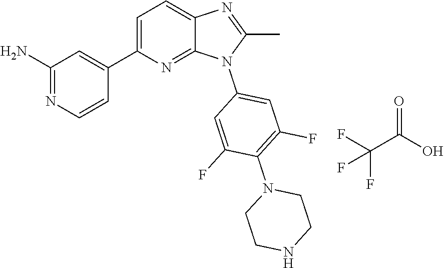

### 4-(3-(3,5-difluoro-4-(piperazin-1-yl)phenyl)-2-methyl-3H-imidazo[4,5-b]pyridin-5-yl)pyridin-2-amine, Trifluoroacetic acid (23d)

Trifluoroacetic acid (0.70 mL, 9.07 mmol, 20 eq.) was added to a 5 mL MWV equipped with a magnetic stir bar containing tert-butyl 4-(4-(5-(2-((tert-butoxycarbonyl)amino)pyridin-4-yl)-2-methyl-3H-imidazo[4,5-b]pyridin-3-yl)-2,6-difluorophenyl)piperazine-1-carboxylate (23c) (282 mg, 0.45 mmol, 1.0 eq.) in DCM (2.80 mL). The reaction mixture was stirred overnight at room temperature where it was then diluted with DCM and concentrated under reduced pressure (3×) to give 4-(3-(3,5-difluoro-4-(piperazin-1-yl)phenyl)-2-methyl-3H-imidazo[4,5-b]pyridin-5-yl)pyridin-2-amine, Trifluoroacetic acid (23d) (228 mg, 0.45 mmol, 93%), a brown solid. LCMS (ESI) C₂₂H₂₁F₂N₇ requires 421.18, found 422 (M/Z+H).

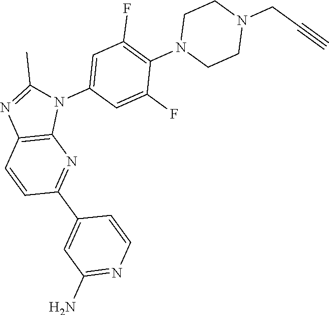 [SMILES: C#CCN1CCN(c2c(F)cc(-n3c(C)nc4ccc(-c5ccnc(N)c5)nc43)cc2F)CC1]

### 4-(3-(3,5-difluoro-4-(4-(prop-2-yn-1-yl)piperazin-1-yl)phenyl)-2-methyl-3H-imidazo[4,5-b]pyridin-5-yl)pyridin-2-amine (23e)

3-bromoprop-1-yne (18 mg, 0.12 mmol, 1.0 eq., 80% Wt.) was added to a 5 mL MWV equipped with a magnetic stir bar containing 4-(3-(3,5-difluoro-4-(piperazin-1-yl)phenyl)-2-methyl-3H-imidazo[4,5-b]pyridin-5-yl)pyridin-2-amine (23d) (50 mg, 0.12 mmol, 1.0 eq.) and potassium carbonate (49 mg, 0.36 mmol, 3.0 eq.) in DMF (1.20 mL). The reaction mixture was heated at 80° C. for 16 hours where it was then cooled down to room temperature and washed with saturated sodium bicarbonate (5 mL). The mixture was extracted with DCM (3×15 mL) and the organic layers were recombined, dried over sodium sulfate, and concentrated under reduced pressure. The crude residue was purified by automated flash column chromatography using a DCM/MeOH gradient to afford 4-(3-(3,5-difluoro-4-(4-(prop-2-yn-1-yl)piperazin-1-yl)phenyl)-2-methyl-3H-imidazo[4,5-b]pyridin-5-yl)pyridin-2-amine (23e) (24 mg, 46 μmol, 39%, 85% purity). LCMS (ESI) C₂₅H₂₃F₂N₇ requires 459.19, found 460. ((M/Z)+H).

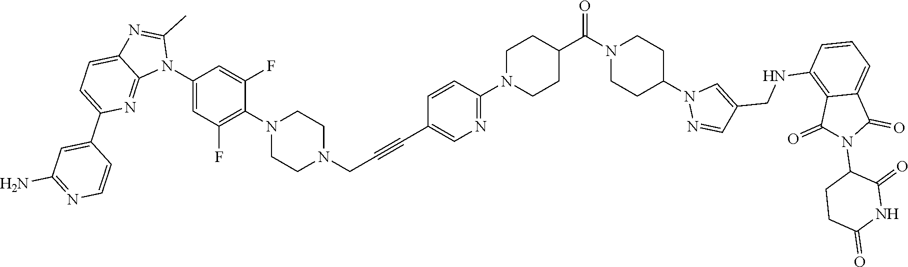 [SMILES: Cc1nc2ccc(-c3ccnc(N)c3)nc2n1-c1cc(F)c(N2CCN(CC#Cc3ccc(N4CCC(C(=O)N5CCC(n6cc(CNc7cccc8c7C(=O)N(C7CCC(=O)NC7=O)C8=O)cn6)CC5)CC4)nc3)CC2)c(F)c1]

### 4-(((1-(1-(1-(5-(3-(4-(4-(5-(2-aminopyridin-4-yl)-2-methyl-3H-imidazo[4,5-b]pyridin-3-yl)-2,6-difluorophenyl)piperazin-1-yl)prop-1-yn-1-yl)pyridin-2-yl)piperidine-4-carbonyl)piperidin-4-yl)-1H-pyrazol-4-yl)methyl)amino)-2-(2,6-dioxopiperidin-3-yl)isoindoline-1,3-dione (23)

A flame dried 5 ml MWV equipped with a magnetic stir bar containing copper (I) iodide (1.0 mg, 3.8 μmol, 0.17 Eq,), PdCl2 (dppf) (2.8 mg, 3.8 μmol, 0.12 eq.) 4-(3-(3,5-difluoro-4-(4-(prop-2-yn-1-yl)piperazin-1-yl)phenyl)-2-methyl-3H-imidazo[4,5-b]pyridin-5-yl)pyridin-2-amine (23e) (17 mg, 31 μmol, 1.0 eq.), and 2-(2,6-dioxopiperidin-3-yl)-4-(((1-(1-(1-(5-iodopyridin-2-yl)piperidine-4-carbonyl)piperidin-4-yl)-1H-pyrazol-4-yl)methyl)amino)isoindoline-1,3-dione (D7) (24 mg, 31 μmol, 1.0 eq.) was backfilled with argon 3 times. DMF (0.75 mL) and triethylamine (12 mg, 120 μmol, 3.0 eq.) were added sequentially and the reaction mixture was stirred at room temperature for 24 hours. The reaction mixture was concentrated under reduced pressure and purified by automated column chromatography to give 4-(((1-(1-(1-(5-(3-(4-(4-(5-(2-aminopyridin-4-yl)-2-methyl-3H-imidazo[4,5-b]pyridin-3-yl)-2,6-difluorophenyl)piperazin-1-yl)prop-1-yn-1-yl)pyridin-2-yl)piperidine-4-carbonyl)piperidin-4-yl)-1H-pyrazol-4-yl)methyl)amino)-2-(2,6-dioxopiperidin-3-yl)isoindoline-1,3-dione (23) (22.7 mg, 3.1 μmol, 67%), a yellow solid. LCMS (ESI) C₅₈H₅₇F₂N₁₅O₅ requires 1082.46, found 1082 (M/Z+H).

### Example 24. Sythesis of 4-(((1-(1-(1-(5-(3-(4-(4-(5-(2-aminopyridin-4-yl)-2-methyl-3H-imidazo[4,5-b]pyridin-3-yl)-2-(trifluoromethyl)phenyl)piperazin-1-yl)prop-1-yn-1-yl)pyridin-2-yl)piperidine-4-carbonyl)piperidin-4-yl)-1H-pyrazol-4-yl)methyl)amino)-2-(2,6-dioxopiperidin-3-yl)isoindoline-1,3-dione (24)

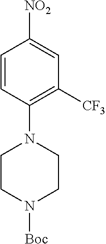 [SMILES: CC(C)(C)OC(=O)N1CCN(c2ccc([N+](=O)[O-])cc2C(F)(F)F)CC1]

### tert-butyl 4-(4-nitro-2-(trifluoromethyl)phenyl)piperazine-1-carboxylate (24a)

To a 5 mL MWV equipped with a magnetic stir bar containing tert-butyl piperazine-1-carboxylate (890 mg, 4.78 mmol, 1.0 eq.), potassium carbonate (1.98 g, 14.35 mmol, 3.0 eq.), in DMF (4.8 mL) was added 1-fluoro-4-nitro-2-(trifluoromethyl)benzene (0.65 mL, 4.78 mmol, 1.0 eq.). The reaction mixture was stirred at 90° C. for 18 hours where the resulting mixture was cooled to room temperature and extracted with ethyl acetate (2×20 mL) and washed with water. The organic layer was then washed with water and brine (5×10 mL) to get rid of DMF where the organic layer was then dried over sodium sulfate, filtered off, and concentrated under reduced pressure and purified by flash column chromatography to give tert-butyl 4-(4-nitro-2-(trifluoromethyl)phenyl)piperazine-1-carboxylate (24a) (884 mg, 2.36 mmol, 49%), an orange solid. ¹H NMR (400 MHz, CDCl₃) δ 8.53 (d, J=2.7 Hz, 1H), 8.35 (dd, J=8.9, 2.7 Hz, 1H), 7.30 (d, J=9.0 Hz, 1H), 3.63-3.56 (m, 4H), 3.09-3.02 (m, 4H), 1.49 (s, 9H). LCMS (ESI) C₁₆H₂₀F₃N₃₀₄ requires 375.14, found 320 (M/Z+H), the carbamic acid adduct.

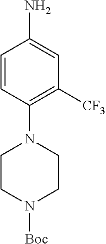 [SMILES: CC(C)(C)OC(=O)N1CCN(c2ccc(N)cc2C(F)(F)F)CC1]

### tert-butyl 4-(4-amino-2-(trifluoromethyl)phenyl)piperazine-1-carboxylate (24b)

To a 20 mL MWV equipped with a magnetic stir bar containing tert-butyl 4-(4-nitro-2-(trifluoromethyl)phenyl)piperazine-1-carboxylate (24a) (853 mg, 2.27 mmol, 1.0 eq.) in Ethanol (14.3 mL) and Water (3.6 mL) was added ammonium chloride (72 mg, 1.36 mmol, 0.6 eq.) and was stirred at 40° C. Iron (761 mg, 13.6 mmol, 6.0 eq.) was then added and heated to 90° C. and stirred for 24 hours. After cooling to room temperature, the reaction mixture was filtered and partitioned between DCM and a saturated solution of sodium bicarbonate. The aqueous phase was extracted with DCM (3×) and the combined organic phases were dried over sodium sulfate. The residue was re-dissolved in chloroform and concentrated under reduced pressure (3×) to give tert-butyl 4-(4-amino-2-(trifluoromethyl)phenyl)piperazine-1-carboxylate (24b) (758 mg, 2.19 mmol, 96%). ¹H NMR (400 MHz, CDCl₃) δ 7.13 (d, J=8.5 Hz, 1H), 6.91 (s, 1H), 6.79 (d, J=8.5 Hz, 1H), 3.91-3.35 (m, 6H), 2.76 (t, J=5.0 Hz, 4H), 1.48 (s, 9H). ¹³C NMR (101 MHz, CDCl₃) δ 155.07, 143.98, 143.14 129.12, 128.87, 128.60, 125.48, 118.70, 113.17, 79.73, 77.48, 77.16, 76.84, 53.64, 28.60.

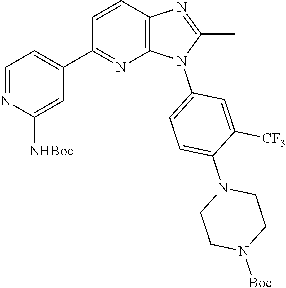 [SMILES: Cc1nc2ccc(-c3ccnc(NC(=O)OC(C)(C)C)c3)nc2n1-c1ccc(N2CCN(C(=O)OC(C)(C)C)CC2)c(C(F)(F)F)c1]

tert-butyl 4-(4-(5-(2-((tert-butoxycarbonyl)amino)pyridin-4-yl)-2-methyl-3H-imidazo[4,5-b]pyridin-3-yl)-2-(trifluoromethyl)phenyl)piperazine-1-carboxylate (24c) potassium phosphate (263 mg, 1.24 mmol, 3.0 eq.), tert-butyl (5-acetamido-6-chloro-[2,4′-bipyridin]-2′-yl)carbamate (A5) (150 mg, 0.41 mmol, 1.0 eq.), tert-butyl 4-(4-amino-2-(trifluoromethyl)phenyl)piperazine-1-carboxylate (24b) (157 mg, 0.45 mmol, 1.1 eq.), dicyclohexyl(2′,4′,6′-triisopropyl-[1,1′-biphenyl]]-2-yl)phosphane (19.7 mg, 41.3 μmol, 0.1 eq.), and Pd2(dba)3 (7.57 mg, 8.27 μmol, 0.02 eq.) were added to a flame-dried 5 ml MWV containing a flame dried stir bar. The MWV was capped and purged with argon. Degassed t-BuOH was added to the MWV and heated to 90 C in an oil bath overnight. The reaction mixture was allowed to cool to room temperature and diluted with dichloromethane. The diluted mixture was filtered through Celite with the aid of dichloromethane and 5% MeOH in DCM. The filtrate was concentrated under reduced pressure and purified by automated column chromatography using a CombiFlash to give tert-butyl (4-(2-methyl-3-(4-(piperazin-1-yl)-3-(trifluoromethyl)phenyl)-3H-imidazo[4,5-b]pyridin-5-yl)pyridin-2-yl)carbamate (24c) (66 mg, 0.12 mmol, 29%), a tan solid. 57% of the aniline starting material was recovered. ¹H NMR (400 MHz, CDCl₃) δ 8.67 (s, 1H), 8.62-8.57 (m, 1H), 8.35-8.27 (m, 1H), 8.07 (d, J=8.3 Hz, 1H), 7.88 (d, J=8.4 Hz, 1H), 7.81 (d, J=2.5 Hz, 1H), 7.72 (dd, J=8.5, 2.6 Hz, 1H), 7.58 (dd, J=5.3, 1.6 Hz, 1H), 7.55 (d, J=8.6 Hz, 1H), 3.61 (t, J=4.9 Hz, 4H), 2.99 (t, J=5.0 Hz, 4H), 2.65 (s, 3H), 1.54 (s, 9H), 1.50 (s, 9H).

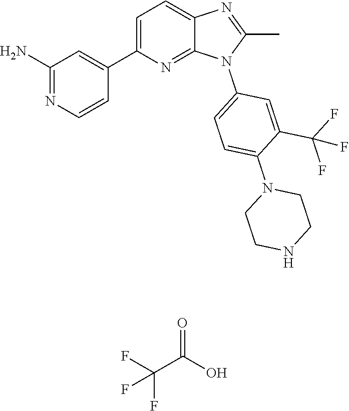 [SMILES: Cc1nc2ccc(-c3ccnc(N)c3)nc2n1-c1ccc(N2CCNCC2)c(C(F)(F)F)c1 ; O=C(O)C(F)(F)F]

4-(2-methyl-3-(4-(piperazin-1-yl)-3-(trifluoromethyl)phenyl)-3H-imidazo[4,5-b]pyridin-5-yl)pyridin-2-amine, trifluoroacetic acid (24d)

Trifluoroacetic acid (0.15 mL, 2.04 mmol, 20 eq.) was added dropwise to a 5 mL MWV equipped with a magnetic stir bar containing tert-butyl 4-(4-(5-(2-((tert-butoxycarbonyl)amino)pyridin-4-yl)-2-methyl-3H-imidazo[4,5-b]pyridin-3-yl)-2-(trifluoromethyl)phenyl)piperazine-1-carboxylate (24c) (66.7 mg, 102 μmol, 1.0 eq.) in DCM (0.75 mL). The reaction mixture was stirred overnight where it was then diluted with DCM and concentrated under reduced pressure (3×) to give 4-(2-methyl-3-(4-(piperazin-1-yl)-3-(trifluoromethyl)phenyl)-3H-imidazo[4,5-b]pyridin-5-yl)pyridin-2-amine, trifluoroacetic acid (24d) (47 mg, 83 μmol, 81%), a brown solid. LCMS (ESI) C₂₃H₂₂F₃N₇ requires 453.47, found 454.29 (M/Z+H).

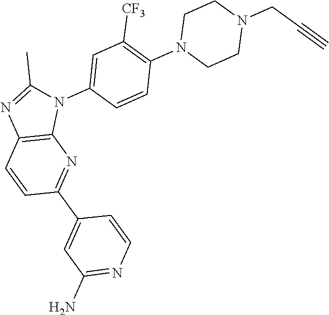 [SMILES: C#CCN1CCN(c2ccc(-n3c(C)nc4ccc(-c5ccnc(N)c5)nc43)cc2C(F)(F)F)CC1]

4-(2-methyl-3-(4-(4-(prop-2-yn-1-yl)piperazin-1-yl)-3-(trifluoromethyl)phenyl)-3H-imidazo[4,5-b]pyridin-5-yl)pyridin-2-amine (24e) 3-bromoprop-1-yne (10 mg, 68 μmol, 1.0 eq., 80% Wt.) was added to a 5 mL MWV equipped with a magnetic stir bar containing 4-(2-methyl-3-(4-(piperazin-1-yl)-3-(trifluoromethyl)phenyl)-3H-imidazo[4,5-b]pyridin-5-yl)pyridin-2-amine (24d) (31 mg, 68 μmol, 1.0 eq.) and potassium carbonate (28 mg, 0.21 mmol, 3.0 eq.) in DMF. The reaction mixture was heated at 80° C. for 16 hours where it was then cooled down to room temperature and washed with saturated sodium bicarbonate (5 mL). The mixture was extracted with DCM (3×15 mL) and the organic layers were recombined, dried over sodium sulfate, and concentrated under reduced pressure. The crude residue was purified by automated flash column chromatography using a DCM/MeOH gradient to afford 4-(2-methyl-3-(4-(4-(prop-2-yn-1-yl)piperazin-1-yl)-3-(trifluoromethyl)phenyl)-3H-imidazo[4,5-b]pyridin-5-yl)pyridin-2-amine (24e) (11 mg, 22 μmol, 33%). LCMS (ESI) C₂₆H₂₄F₃N₇ requires 491.20, found 492 (M/Z+H).

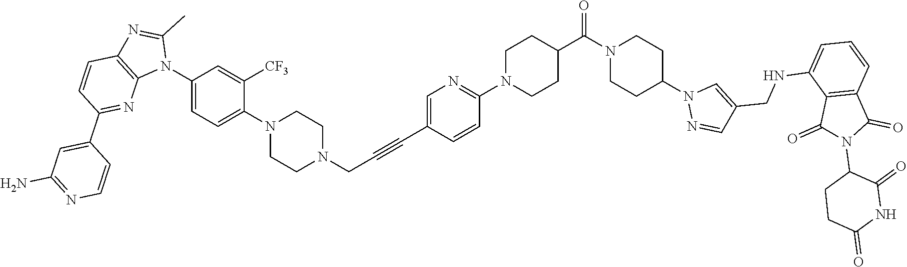 [SMILES: Cc1nc2ccc(-c3ccnc(N)c3)nc2n1-c1ccc(N2CCN(CC#Cc3ccc(N4CCC(C(=O)N5CCC(n6cc(CNc7cccc8c7C(=O)N(C7CCC(=O)NC7=O)C8=O)cn6)CC5)CC4)nc3)CC2)c(C(F)(F)F)c1]

4-(((1-(1-(1-(5-(3-(4-(4-(5-(2-aminopyridin-4-yl)-2-methyl-3H-imidazo[4,5-b]pyridin-3-yl)-2-(trifluoromethyl)phenyl)piperazin-1-yl)prop-1-yn-1-yl)pyridin-2-yl)piperidine-4-carbonyl)piperidin-4-yl)-1H-pyrazol-4-yl)methyl)amino)-2-(2,6-dioxopiperidin-3-yl)isoindoline-1,3-dione (24)

A flame dried 5 ml MWV equipped with a magnetic stir bar containing copper (I) iodide (0.6 mg, 3.0 μmol, 0.20 Eq,), PdCl2 (dppf) (1.8 mg, 2.5 μmol, 0.13 Eq.) 4-(2-methyl-3-(4-(4-(prop-2-yn-1-yl)piperazin-1-yl)-3-(trifluoromethyl)phenyl)-3H-imidazo[4,5-b]pyridin-5-yl)pyridin-2-amine (24e) (9 mg, 18 μmol, 1.0 eq.), and 2-(2,6-dioxopiperidin-3-yl)-4-(((1-(1-(1-(5-iodopyridin-2-yl)piperidine-4-carbonyl)piperidin-4-yl)-1H-pyrazol-4-yl)methyl)amino)isoindoline-1,3-dione (D7) (14 mg, 18 μmol, 1.0 eq.) was backfilled with argon 3 times. DMF (0.70 mL) and diisopropylethylamine (7.1 mg, 55 μmol, 3.0 eq.) were added sequentially and the reaction mixture was stirred at room temperature for 24 hours. The reaction mixture was concentrated under reduced pressure and purified by automated column chromatography to give 4-(((1-(1-(1-(5-(3-(4-(4-(5-(2-aminopyridin-4-yl)-2-methyl-3H-imidazo[4,5-b]pyridin-3-yl)-2-(trifluoromethyl)phenyl)piperazin-1-yl)prop-1-yn-1-yl)pyridin-2-yl)piperidine-4-carbonyl)piperidin-4-yl)-1H-pyrazol-4-yl)methyl)amino)-2-(2,6-dioxopiperidin-3-yl)isoindoline-1,3-dione (24) (5 mg, 4 μmol, 20%), a yellow solid. ¹H NMR (500 MHz, CDCl₃) δ 8.18-8.05 (m, 1H), 8.00-7.91 (m, 1H), 7.79 (s, 1H), 7.74-7.60 (m, 1H), 7.60-7.48 (m, 1H), 7.37 (ddt, J=16.0, 11.7, 5.4 Hz, 4H), 7.26 (d, J=1.8 Hz, 1H), 7.24 (d, J=4.3 Hz, 3H), 7.13 (s, 1H), 7.05-6.94 (m, 1H), 6.86-6.77 (m, 1H), 6.56-6.45 (m, 1H), 4.85-4.70 (m, 1H), 4.56 (s, 1H), 4.33-4.17 (m, 6H), 3.61-3.40 (m, 4H), 3.30-3.18 (m, 7H), 3.04 (q, J=5.4 Hz, 4H), 2.91-2.57 (m, 3H), 2.52-2.45 (m, 3H), 2.12 (s, 2H), 1.73-1.60 (m, 5H). ¹⁹F NMR (471 MHz, CDCl₃) δ -56.72,−56.73. Note: NH₂ did not appear in CDCl₃ and in ¹⁹F There are two peaks under 56.72. LCMS (ESI) C₅₉H₅₈F₃N₁₅O₅ requires 1113.46, found 1114 (M/Z+H).

### Example 25. Synthesis of 4-(((1-(1-(1-(5-(3-(4-(4-(5-(2-aminopyridin-4-yl)-2-methyl-3H-imidazo[4,5-b]pyridin-3-yl)benzyl)piperazin-1-yl)prop-1-yn-1-yl)pyridin-2-yl)piperidine-4-carbonyl)piperidin-4-yl)-1H-pyrazol-4-yl)methyl)amino)-2-(2,6-dioxopiperidin-3-yl)isoindoline-1,3-dione (25)

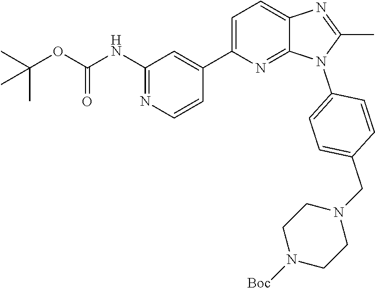 [SMILES: Cc1nc2ccc(-c3ccnc(NC(=O)OC(C)(C)C)c3)nc2n1-c1ccc(CN2CCN(C(=O)OC(C)(C)C)CC2)cc1]

### tert-butyl 4-(4-(5-(2-((tert-butoxycarbonyl)amino)pyridin-4-yl)-2-methyl-3H-imidazo[4,5-b]pyridin-3-yl)benzyl)piperazine-1-carboxylate (25a)

Potassium phosphate (176 mg, 0.82 mmol, 3.0 eq.), tert-butyl (5-acetamido-6-chloro-[2,4′-bipyridin]-2′-yl)carbamate (A5) (100 mg, 276 μmol, 1.0 eq.), tert-butyl 4-(4-aminobenzyl)piperazine-1-carboxylate (88.3 mg, 303 μmol, 1.1 eq.), dicyclohexyl(2′,4′,6′-triisopropyl-[1,1′-biphenyl]-2-yl)phosphane (13.1 mg, 27.6 μmol, 0.10 eq.), and Pd2(dba)3 (5.05 mg, 5.51 μmol, 0.02 eq.) were added to a flame-dried 5 ml MWV containing a flame dried stir bar. The MWV was capped and purged with argon where tert-butanol (1.00 mL) was added and the reaction mixture was stirred at 90° C. for 24 hours. The reaction mixture was cooled to room temperature, diluted with 5% MeOH in DCM and filtered through celite. The celite pad was further washed with this solution and the filtrate was concentrated under reduced pressure and purified by flash column chromatography to give tert-butyl 4-(4-(5-(2-((tert-butoxycarbonyl)amino)pyridin-4-yl)-2-methyl-3H-imidazo[4,5-b]pyridin-3-yl)benzyl)piperazine-1-carboxylate (25a) (150 mg, 0.25 mmol, 90%, eluting at 5% MeOH in DCM), a brown solid. ¹H NMR (500 MHz, CDCl₃) δ 8.54 (s, 1H), 8.42 (s, 1H), 8.30 (s, 1H), 8.06 (d, J=8.3 Hz, 1H), 7.87 (d, J=8.0 Hz, 1H), 7.68-7.53 (m, 3H), 7.53-7.42 (m, 2H), 3.65 (s, 2H), 3.50 (s, 4H), 2.61 (s, 3H), 2.51 (s, 4H), 1.54 (s, 9H), 1.46 (s, 9H). ¹³C NMR (126 MHz, CDCl₃) δ 154.86, 152.97, 152.75, 149.15, 148.24, 130.27, 127.26, 127.06, 116.90, 116.51, 109.80, 80.98, 79.88, 77.41, 77.16, 76.91, 62.52, 29.82, 28.55, 28.48, 25.89, 15.66.

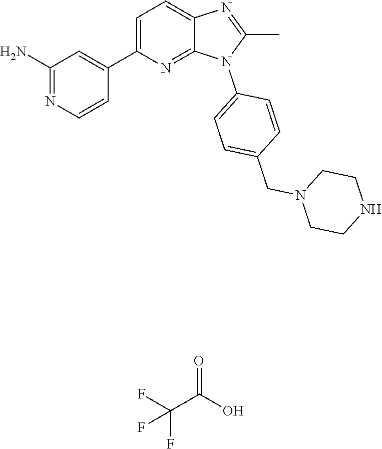 [SMILES: Cc1nc2ccc(-c3ccnc(N)c3)nc2n1-c1ccc(CN2CCNCC2)cc1 ; O=C(O)C(F)(F)F]

### 4-(2-methyl-3-(4-(piperazin-1-ylmethyl)phenyl)-3H-imidazo[4,5-b]pyridin-5-yl)pyridin-2-amine, Trifluoroacetic acid (25b)

Trifluoroacetic acid (0.39 mL, 5.00 mmol, 20 eq.) was added to a solution of tert-butyl 4-(4-(5-(2-((tert-butoxycarbonyl)amino)pyridin-4-yl)-2-methyl-3H-imidazo[4,5-b]pyridin-3-yl)benzyl)piperazine-1-carboxylate (25a) (150 mg, 0.25 mmol, 1.0 eq.) in DCM (1.20 mL). The reaction mixture was stirred at room temperature overnight where it was diluted with DCM and concentrated under reduced pressure. The crude residue was purified by flash column chromatography to give 4-(2-methyl-3-(4-(piperazin-1-ylmethyl)phenyl)-3H-imidazo[4,5-b]pyridin-5-yl)pyridin-2-amine, Trifluoroacetic acid (25b) (54 mg, 0.11 mmol, 42%, eluting at 20% MeOH in DCM), a red semisolid. ¹H NMR (400 MHz, DMSO) δ 8.20 (d, J=8.3 Hz, 1H), 8.09 (bs, 2H), 8.00 (t, J=7.6 Hz, 2H), 7.61 (s, 4H), 7.49 (d, J=1.7 Hz, 1H), 7.38 (dd, J=6.6, 1.7 Hz, 1H), 3.72 (s, 2H), 3.21-3.14 (m, 4H), 2.70 (t, J=5.2 Hz, 4H), 2.54-2.49 (m, 4H). ¹³C NMR (101 MHz, DMSO) δ 155.89, 155.59, 151.48, 148.83, 145.42, 138.06, 135.83, 133.14, 129.89, 127.30, 126.91, 117.09, 109.67, 108.60, 60.81, 49.21, 48.58, 42.88, 15.08.

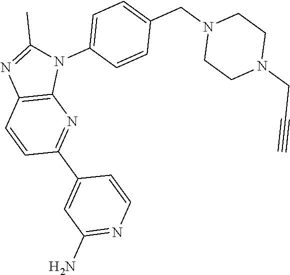 [SMILES: C#CCN1CCN(Cc2ccc(-n3c(C)nc4ccc(-c5ccnc(N)c5)nc43)cc2)CC1]

### 4-(2-methyl-3-(4-((4-(prop-2-yn-1-yl)piperazin-1-yl)methyl)phenyl)-3H-imidazo[4,5-b]pyridin-5-yl)pyridin-2-amine (25c)

3-bromoprop-1-yne (24 mg, 0.16 mmol, 1.2 eq., 80% Wt.) was added to a 5 mL MWV equipped with a magnetic stir bar containing 4-(2-methyl-3-(4-(piperazin-1-ylmethyl)phenyl)-3H-imidazo[4,5-b]pyridin-5-yl)pyridin-2-amine (25b) (54 mg, 0.14 mmol, 1.0 eq.) and potassium carbonate (56 mg, 0.41 mmol, 3.0 eq.) in DMF (1.4 mL). The reaction mixture was heated at 90° C. for 16 hours where it was then cooled down to room temperature and washed with saturated sodium bicarbonate (5 mL). The mixture was extracted with DCM (3×15 mL) and the organic layers were recombined, dried over sodium sulfate, and concentrated under reduced pressure. The crude residue was purified by flash column chromatography using a DCM/MeOH gradient to afford 4-(2-methyl-3-(4-((4-(prop-2-yn-1-yl)piperazin-1-yl)methyl)phenyl)-3H-imidazo[4,5-b]pyridin-5-yl)pyridin-2-amine (25c) (20.8 mg, 47.5 μmol, 35%). LCMS (ESI) C₂₆H₂₇N₇ requires 437.23, found 438 (M/Z+H).

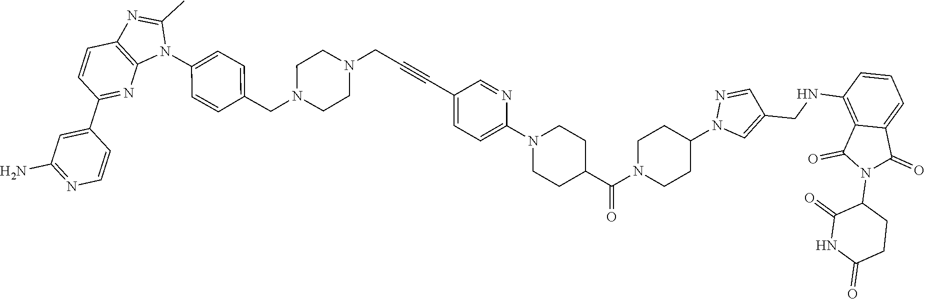 [SMILES: Cc1nc2ccc(-c3ccnc(N)c3)nc2n1-c1ccc(CN2CCN(CC#Cc3ccc(N4CCC(C(=O)N5CCC(n6cc(CNc7cccc8c7C(=O)N(C7CCC(=O)NC7=O)C8=O)cn6)CC5)CC4)nc3)CC2)cc1]

### 4-(((1-(1-(1-(5-(3-(4-(4-(5-(2-aminopyridin-4-yl)-2-methyl-3H-imidazo[4,5-b]pyridin-3-yl)benzyl)piperazin-1-yl)prop-1-yn-1-yl)pyridin-2-yl)piperidine-4-carbonyl)piperidin-4-yl)-1H-pyrazol-4-yl)methyl)amino)-2-(2,6-dioxopiperidin-3-yl)isoindoline-1,3-dione (25)

A flame dried 5 ml MWV equipped with a magnetic stir bar containing copper (I) iodide (1.0 mg, 5.5 μmol, 0.12 eq.), PdCl2 (dppf) (4.0 mg, 5.5 μmol, 0.12 eq.) 4-(2-methyl-3-(4-((4-(prop-2-yn-1-yl)piperazin-1-yl)methyl)phenyl)-3H-imidazo[4,5-b]pyridin-5-yl)pyridin-2-amine (25c) (20 mg, 46 μmol, 1.0 eq.), and 2-(2,6-dioxopiperidin-3-yl)-4-(((1-(1-(1-(5-iodopyridin-2-yl)piperidine-4-carbonyl)piperidin-4-yl)-1H-pyrazol-4-yl)methyl)amino)isoindoline-1,3-dione (D7) (34 mg, 46 μmol, 1.0 eq.) was backfilled with argon 3 times. DMF (0.75 mL) and triethylamine (14 mg, 140 μmol, 3.0 eq.) were added sequentially and the reaction mixture was stirred at room temperature for 24 hours. The reaction mixture was concentrated under reduced pressure and purified by automated column chromatography to give 4-(((1-(1-(1-(5-(3-(4-(4-(5-(2-aminopyridin-4-yl)-2-methyl-3H-imidazo[4,5-b]pyridin-3-yl)benzyl)piperazin-1-yl)prop-1-yn-1-yl)pyridin-2-yl)piperidine-4-carbonyl)piperidin-4-yl)-1H-pyrazol-4-yl)methyl)amino)-2-(2,6-dioxopiperidin-3-yl)isoindoline-1,3-dione (25) (21 mg, 29 μmol, 44%), a yellow solid. LCMS (ESI) C₅₉H₆₁N₁₅O₅ requires 1059.49, found 1060 (M/2).

### Example 26. Synthesis of 4-(((1-(1-(1-(5-(4-(4-(5-(2-aminopyridin-4-yl)-2-methyl-3H-imidazo[4,5-b]pyridin-3-yl)-2-fluorophenyl)piperazin-1-yl)pyridin-2-yl)piperidine-4-carbonyl)piperidin-4-yl)-1H-pyrazol-4-yl)methyl)amino)-2-(2,6-dioxopiperidin-3-yl)isoindoline-1,3-dione (26)

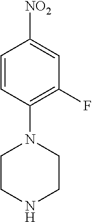 [SMILES: O=[N+]([O-])c1ccc(N2CCNCC2)c(F)c1]

### 1-(2-fluoro-4-nitrophenyl)piperazine, trifluoroacetic acid (26a)

To a 250 mL RBF equipped with a stir bar containing piperazine (5.41 g, 62.85 mmol, 2.5 eq.) in Acetonitrile (42 mL) was added 1,2-difluoro-4-nitrobenzene (2.78 mL, 25.14 mmol, 1.0 eq.) dropwise. The reaction mixture was reflux at 80° C. for 24 hours. After cooling down the room temperature, the reaction mixture was concentrated under reduced pressure, extracted with ethyl acetate (3×30 ml) and washed with brine. The organic layers were recombined, dried over sodium sulfate, and concentrated under reduced pressure. The crude residue was purified by flash column chromatography to give 1-(2-fluoro-4-nitrophenyl)piperazine (26a) (5.584 g, 24.79 mmol, 98%, eluting at 6% MeOH in DCM), a yellow solid. ¹H NMR (500 MHz, CDCl₃) δ 7.99-7.93 (m, 1H), 7.91-7.84 (m, 1H), 6.89 (t, J=8.8 Hz, 1H), 3.28-3.22 (m, 4H), 3.06-3.00 (m, 4H), 1.68 (s, 1H). ¹³C NMR (126 MHz, CDCl₃) δ 153.14 (d, J=249.2 Hz), 146.14 (d, J=7.6 Hz), 140.48 (d, J=8.7 Hz), 121.11 (d, J=2.8 Hz), 117.11 (d, J=4.1 Hz), 112.66 (d, J=26.5 Hz), 50.99 (d, J=5.0 Hz), 46.05.

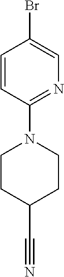 [SMILES: N#CC1CCN(c2ccc(Br)cn2)CC1]

### 1-(5-bromopyridin-2-yl)piperidine-4-carbonitrile (26b)

5-bromo-2-fluoropyridine (0.75 mL, 7.26 mmol, 1.0 eq.) and piperidine-4-carbonitrile (0.81 mL, 7.26 mmol, 1.0 eq.) were added to a 24 mL MWV equipped with a magnetic stir bar containing potassium carbonate (2.01 g, 14.5 mmol, 2.0 eq.) and DMF (17 mL). The reaction mixture was stirred at 90° C. for 24 hours where it was then quenched with water and extracted with ethyl acetate (3×15 mL). The organic layers were recombined and washed with water (5×). The organic layer was then dried over sodium sulfate and concentrated under reduced pressure. The crude residue was purified by flash column chromatography to give 1-(5-bromopyridin-2-yl)piperidine-4-carbonitrile (26b) (982 mg, 3.69 mmol, 50%), a white solid. LCMS (ESI) C₁₁H₁₂BrN₃ requires 265.02, found 265 (M/Z+H).

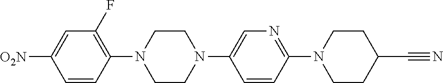 [SMILES: N#CC1CCN(c2ccc(N3CCN(c4ccc([N+](=O)[O-])cc4F)CC3)cn2)CC1]

### 1-(5-(4-(2-fluoro-4-nitrophenyl)piperazin-1-yl)pyridin-2-yl)piperidine-4-carbonitrile (26c)

To a dry 20 mL MWV equipped with a magnetic stir bar was added 1-(2-fluoro-4-nitrophenyl)piperazine (26a) (243 mg, 1.08 mmol, 1.15 eq.), 1-(5-bromopyridin-2-yl)piperidine-4-carbonitrile (26b) (250 mg, 0.93 mmol, 1.0 eq.), Pd2(dba)3 (68 mg, 75 μmol, 0.08 eq.), sodium tert-butoxide (271 mg, 2.82 mmol, 3.0 eq.), and Xantphos (87 mg, 150 μmol, 0.16 eq.). The MWV was capped and purged with argon for 15 minutes where toluene (4.30 mL) was added. The reaction mixture was stirred at 90° C. for 16 hours and was allowed to warm to room temperature, filtered through celite. The celite filter pad was washed with DCM and the filtrate was extracted with water, brine, and ethyl acetate. The organic layers were recombined, dried over sodium sulfate, filtered off, and concentrated under reduced pressure. The crude residue was purified by flash column chromatography to give 1-(5-(4-(2-fluoro-4-nitrophenyl)piperazin-1-yl)pyridin-2-yl)piperidine-4-carbonitrile (26c) (133 mg, 0.32 mmol, 34%, eluting at 100% DCM), a red semisolid. ¹H NMR (400 MHz, CDCl₃) δ 8.04 (ddd, J=9.0, 2.6, 1.0 Hz, 1H), 7.99 (d, J=3.0 Hz, 1H), 7.98 (d, J=2.6 Hz, OH), 7.95 (d, J=2.6 Hz, OH), 7.32 (dd, J=9.2, 3.1 Hz, 1H), 7.00 (t, J=8.8 Hz, 1H), 6.76-6.64 (m, 1H), 3.80 (ddd, J=13.3, 7.0, 3.6 Hz, 2H), 3.54-3.46 (m, 4H), 3.42 (ddd, J=13.3, 7.9, 3.5 Hz, 1H), 3.30-3.22 (m, 4H), 2.88 (tq, J=8.2, 3.6 Hz, 1H), 2.13-1.91 (m, 5H).

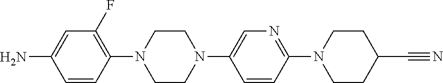 [SMILES: N#CC1CCN(c2ccc(N3CCN(c4ccc(N)cc4F)CC3)cn2)CC1]

### 1-(5-(4-(4-amino-2-fluorophenyl)piperazin-1-yl)pyridin-2-yl)piperidine-4-carbonitrile (26d)

To a 8 mL MWV equipped with a magnetic stir bar containing 1-(5-(4-(2-fluoro-4-nitrophenyl)piperazin-1-yl)pyridin-2-yl)piperidine-4-carbonitrile (26c) (133 mg, 324 μmol, 1.0 eq.) in ethanol (3.24 mL) and water (810 μL) was added ammonium chloride (10.4 mg, 194 μmol, 0.6 eq.) and was stirred at 40 C. iron (109 mg, 1.94 mmol, 6.0 eq.) was then added and heated to 90° C. and stirred for 6 hours. After cooling to room temperature, the reaction mixture was filtered and partitioned between DCM and saturated sodium bicarbonate. The aqueous phase was extracted with DCM (3 x) and the combined organic phases were dried over sodium sulfate. The residue was redissolved in chloroform and concentrated under reduced pressure (3 x) to give 1-(5-(4-(4-amino-2-fluorophenyl)piperazin-1-yl)pyridin-2-yl)piperidine-4-carbonitrile (26d) (140 mg, 0.32 mmol, 100%, 88% Purity). LCMS (ESI) C₂₁H₂₅FN₆ requires 380.21, found 381 (M/Z+H).

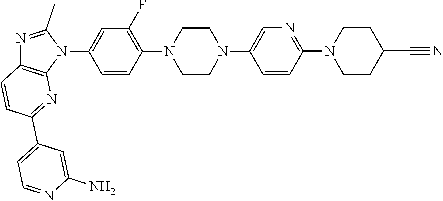 [SMILES: Cc1nc2ccc(-c3ccnc(N)c3)nc2n1-c1ccc(N2CCN(c3ccc(N4CCC(C#N)CC4)nc3)CC2)c(F)c1]

### 1-(5-(4-(4-(5-(2-aminopyridin-4-yl)-2-methyl-3H-imidazo[4,5-b]pyridin-3-yl)-2-fluorophenyl)piperazin-1-yl)pyridin-2-yl)piperidine-4-carbonitrile (26e)

To a 5 mL MWV equipped with a magnetic stir bar containing tert-butyl (5-acetamido-6-chloro-[2,4′-bipyridin]-2′-yl)carbamate (A5) (107 mg, 295 μmol, 1.0 eq.), 1-(5-(4-(4-amino-2-fluorophenyl)piperazin-1-yl)pyridin-2-yl)piperidine-4-carbonitrile (26d) (123 mg, 323 μmol, 1.10 eq.), dicyclohexyl(2′,4′,6′-triisopropyl-[1,1′-biphenyl]-2-yl)phosphane (14.1 mg, 29.5 μmol, 0.10 eq.), and Pd2(dba)3 (5.40 mg, 5.90 μmol, 0.02 eq.), and potassium phosphate (188 mg, 885 μmol, 3.0 eq.) was added tert-butanol (1.08 mL) and the reaction mixture was stirred at 90° C. for 24 hours. The reaction mixture was allowed to cool to room temperature, diluted with DCM, and filtered through celite. The celite pad was washed with a solution of 5% MeOH in DCM. The filtrate was concentrated under reduced pressure and purified by flash column chromatography to give-(5-(4-(4-(5-(2-aminopyridin-4-yl)-2-methyl-3H-imidazo[4,5-b]pyridin-3-yl)-2-fluorophenyl)piperazin-1-yl)pyridin-2-yl)piperidine-4-carbonitrile (26e) (69 mg, 0.10 mmol, 34%), a brown solid. Note: 40% of the chloro-starting material was recovered. LCMS (ESI) C₃₈H₄₁FN₁₀O₂ requires 688.33, found 689 (M/Z+H).

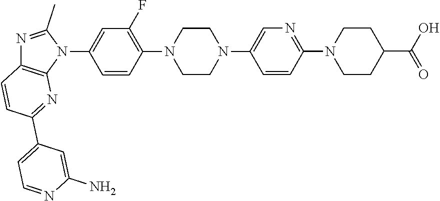 [SMILES: Cc1nc2ccc(-c3ccnc(N)c3)nc2n1-c1ccc(N2CCN(c3ccc(N4CCC(C(=O)O)CC4)nc3)CC2)c(F)c1]

### 1-(5-(4-(4-(5-(2-aminopyridin-4-yl)-2-methyl-3H-imidazo[4,5-b]pyridin-3-yl)-2-fluorophenyl)piperazin-1-yl)pyridin-2-yl)piperidine-4-carboxylic acid (26f)

HCl (1.10 mL, 6.6 mmol, 6M, 61 eq.) was added to a 5 mL MWV equipped with a magnetic stir bar containing tert-butyl (4-(3-(4-(4-(6-(4-cyanopiperidin-1-yl)pyridin-3-yl)piperazin-1-yl)-3-fluorophenyl)-2-methyl-3H-imidazo[4,5-b]pyridin-5-yl)pyridin-2-yl)carbamate (26e) (75 mg, 0.11 mmol, 1.0 eq.) and was stirred at 100° C. for 16 hours where it was then cooled down to room temperature, azeotroped with toluene, and concentrated under reduced pressure to obtain 1-(5-(4-(4-(5-(2-aminopyridin-4-yl)-2-methyl-3H-imidazo[4,5-b]pyridin-3-yl)-2-fluorophenyl)piperazin-1-yl)pyridin-2-yl)piperidine-4-carboxylic acid, HCl (26f), a black semisolid. This crude residue was used without further purification. LCMS (ESI) C₃₃H₃₄FN₉O₂ requires 607.28, found 608 (M/Z+H).

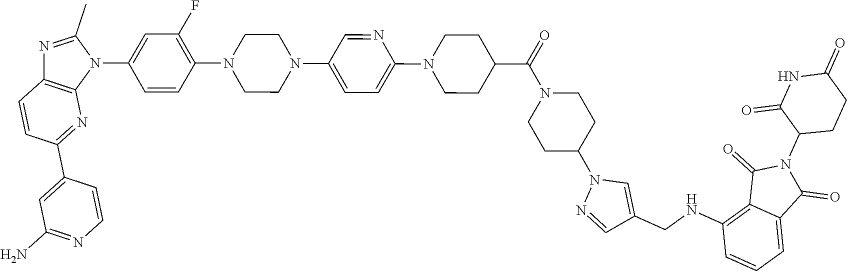 [SMILES: Cc1nc2ccc(-c3ccnc(N)c3)nc2n1-c1ccc(N2CCN(c3ccc(N4CCC(C(=O)N5CCC(n6cc(CNc7cccc8c7C(=O)N(C7CCC(=O)NC7=O)C8=O)cn6)CC5)CC4)nc3)CC2)c(F)c1]

### 4-(((1-(1-(1-(5-(4-(4-(5-(2-aminopyridin-4-yl)-2-methyl-3H-imidazo[4,5-b]pyridin-3-yl)-2-fluorophenyl)piperazin-1-yl)pyridin-2-yl)piperidine-4-carbonyl)piperidin-4-yl)-1H-pyrazol-4-yl)methyl)amino)-2-(2,6-dioxopiperidin-3-yl)isoindoline-1,3-dione (26)

A flame dried 5 ml MWV equipped with a magnetic stir bar containing HATU (25 mg, 65 μmol, 1.2 eq.), 2-(2,6-dioxopiperidin-3-yl)-4-(((1-(piperidin-4-yl)-1H-pyrazol-4-yl)methyl)amino)isoindoline-1,3-dione, Trifluoroacetic acid (D4) (40 mg, 54 μmol, 1.0 eq.), 1-(5-(4-(4-(5-(2-aminopyridin-4-yl)-2-methyl-3H-imidazo[4,5-b]pyridin-3-yl)-2-fluorophenyl)piperazin-1-yl)pyridin-2-yl)piperidine-4-carboxylic acid, Trifluoroacetic acid (26f) (43 mg, 60 μmol, 1.1 eq.) was added DMF (1.00 mL) and diisopropylethylamine (60 μL, 0.33 mmol, 6 eq.). The reaction mixture was stirred at room temperature for 24 hours where it was then concentrated under reduced pressure and purified by flash column chromatography to give 4-(((1-(1-(1-(5-(4-(4-(5-(2-aminopyridin-4-yl)-2-methyl-3H-imidazo[4,5-b]pyridin-3-yl)-2-fluorophenyl)piperazin-1-yl)pyridin-2-yl)piperidine-4-carbonyl)piperidin-4-yl)-1H-pyrazol-4-yl)methyl)amino)-2-(2,6-dioxopiperidin-3-yl)isoindoline-1,3-dione (26) (2.5 mg, 2.4 μmol, 4.5%), a brown solid. LCMS (ESI) C₅₅H₅₆FN₁₅O₅ requires 1026.15, found 514.39 (M/2).

### Example 27. Synthesis of 4-(((1-(1-(1-(5-(3-(4-(4-(5-(2-aminopyridin-4-yl)-2-methyl-3H-imidazo[4,5-b]pyridin-3-yl)-2,6-dichlorophenyl)piperazin-1-yl)prop-1-yn-1-yl)pyridin-2-yl)piperidine-4-carbonyl)piperidin-4-yl)-1H-pyrazol-4-yl)methyl)amino)-2-(2,6-dioxopiperidin-3-yl)isoindoline-1,3-dione (27)

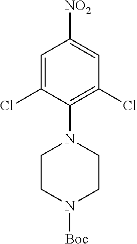 [SMILES: CC(C)(C)OC(=O)N1CCN(c2c(Cl)cc([N+](=O)[O-])cc2Cl)CC1]

### tert-butyl 4-(2,6-dichloro-4-nitrophenyl)piperazine-1-carboxylate (27a)

To a 5 mL MWV equipped with a magnetic stir bar containing tert-butyl piperazine-1-carboxylate (886.9 mg, 1 Eq, 4.762 mmol), potassium carbonate (1.974 g, 3 Eq, 14.29 mmol), in DMF (6.00 mL) was added 1,3-dichloro-2-fluoro-5-nitrobenzene (1.000 g, 1 Eq, 4.762 mmol). The reaction mixture was stirred at 90 C for 18 hours where the resulting mixture was cooled to room temperature and extracted with ethyl acetate (2×20 mL) and washed with water. The organic layer was then washed with water and brine (5×10 mL) to get rid of DMF where the organic layer was then dried over sodium sulfate, filtered off, and concentrated under reduced pressure and purified by flash column chromatography to give tert-butyl 4-(2,6-dichloro-4-nitrophenyl)piperazine-1-carboxylate (933 mg, 2.48 mmol, 52%). ¹H NMR (500 MHz, CDCl₃) δ 8.25 (d, J=1.5 Hz, 1H), 8.16 (d, J=1.6 Hz, 1H), 3.78-3.69 (m, 1H), 3.64-3.51 (m, 5H), 3.27 (t, J=4.9 Hz, 2H), 1.49 (s, 9H).

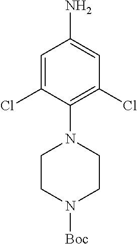 [SMILES: CC(C)(C)OC(=O)N1CCN(c2c(Cl)cc(N)cc2Cl)CC1]

### tert-butyl 4-(4-amino-2,6-dichlorophenyl)piperazine-1-carboxylate (27b)

Methanol (25.00 mL) was added to a dry 250 mL RBF under argon containing tert-butyl 4-(2,6-dichloro-4-nitrophenyl)piperazine-1-carboxylate (27a) (933 mg, 2.48 mmol, 1.0 eq.) and 10% palladium on carbon (92 mg). The reaction mixture was stirred under an atmosphere of hydrogen and stirred overnight where it was then filtered through celite, washed with DCM, and concentrated under reduced pressure. The crude residue was purified by flash column chromatography to give tert-butyl 4-(4-amino-2,6-dichlorophenyl)piperazine-1-carboxylate (27b) (341 mg, 0.98 mmol, 39% yield), an orange semisolid. ¹H NMR (500 MHz, CDCl₃) δ 6.66-6.62 (m, 2H), 3.60-3.46 (m, 6H), 3.07 (t, J=4.7 Hz, 4H), 1.48 (d, J=2.7 Hz, 9H). ¹³C NMR (126 MHz, CDCl₃) δ 155.14, 143.81, 136.66, 136.30, 129.47, 115.72, 114.84, 79.70, 77.41, 77.16, 76.91, 49.62, 28.61, 28.52.

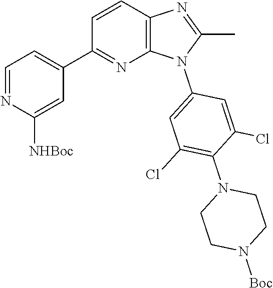 [SMILES: Cc1nc2ccc(-c3ccnc(NC(=O)OC(C)(C)C)c3)nc2n1-c1cc(Cl)c(N2CCN(C(=O)OC(C)(C)C)CC2)c(Cl)c1]

### tert-butyl 4-(4-(5-(2-((tert-butoxycarbonyl)amino)pyridin-4-yl)-2-methyl-3H-imidazo[4,5-b]pyridin-3-yl)-2,6-dichlorophenyl)piperazine-1-carboxylate (27c)

Potassium phosphate (263 mg, 1.24 mmol, 3.0 eq.), tert-butyl (5-acetamido-6-chloro-[2,4′-bipyridin]-2′-yl)carbamate (A5) (150 mg, 413 μmol, 1.0 eq.), tert-butyl 4-(4-amino-2,6-dichlorophenyl)piperazine-1-carboxylate (27b) (157 mg, 455 μmol, 1.1 eq.), dicyclohexyl(2′,4′,6′-triisopropyl-[1,1′-biphenyl]-2-yl)phosphane (19.7 mg, 41 μmol, 0.10 eq.), and Pd2(dba)3 (7.5 mg, 0.02 Eq, 8 μmol) were added to a flame-dried 5 ml MWV containing a flame dried stir bar. The MWV was capped and purged with argon. Degassed t-BuOH (1.50 mL) was added to the MWV and heated to 90° C. in an oil bath. The reaction mixture was allowed to cool to room temperature and diluted with dichloromethane. The diluted mixture was filtered through Celite with the aid of dichloromethane and 5% MeOH in DCM. The filtrate was concentrated under reduced pressure and purified by flash column chromatography to give tert-butyl 4-(4-(5-(2-((tert-butoxycarbonyl)amino)pyridin-4-yl)-2-methyl-3H-imidazo[4,5-b]pyridin-3-yl)-2,6-dichlorophenyl)piperazine-1-carboxylate (27c) (113 mg, 173 μmol, 41%), a brown semisolid. LCMS (ESI) C₃₂H₃₇C₁₂N₇₀₄ requires 653.22, found 654 (M/Z+H).

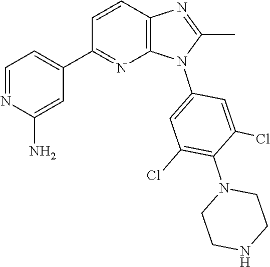 [SMILES: Cc1nc2ccc(-c3ccnc(N)c3)nc2n1-c1cc(Cl)c(N2CCNCC2)c(Cl)c1]

### 4-(3-(3,5-dichloro-4-(piperazin-1-yl)phenyl)-2-methyl-3H-imidazo[4,5-b]pyridin-5-yl)pyridin-2-amine (27d)

TFA (0.37 mL, 4.86 mmol, 20.0 eq.) was added to a 20 mL MWV equipped with a magnetic stir bar containing tert-butyl 4-(4-(5-(2-((tert-butoxycarbonyl)amino)pyridin-4-yl)-2-methyl-3H-imidazo[4,5-b]pyridin-3-yl)-2,6-dichlorophenyl)piperazine-1-carboxylate (27c) (159 mg, 243 μmol, 1.0 eq.) and DCM (1.5 mL). The reaction mixture was stirred overnight where it was then concentrated under reduced pressure and used in the next step without further purification and extracted with DCM and washed with a saturated solution of sodium bicarbonate. The organic layer was dried over magnesium sulfate, filtered, and concentrated under reduced pressure to give4-(3-(3,5-dichloro-4-(piperazin-1-yl)phenyl)-2-methyl-3H-imidazo[4,5-b]pyridin-5-yl)pyridin-2-amine (27d) (60.3 mg, 133 μmol, 54%), a brown solid. ¹H NMR (400 MHz, MeOD) δ 8.17 (dd, J=8.4, 2.7 Hz, 1H), 8.11-8.02 (m, 1H), 7.92-7.84 (m, 2H), 7.75 (s, 1H), 7.68-7.56 (m, 1H), 7.56-7.49 (m, 1H), 3.63 (t, J=5.0 Hz, 3H), 3.44 (t, J=5.1 Hz, 4H), 3.37 (s, 3H), 2.62 (s, 3H). LCMS (ESI) C₂₂H₂₁C₁₂N₇ requires 453.12, found 454 (M/Z+H)

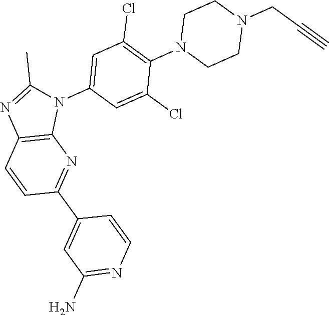 [SMILES: C#CCN1CCN(c2c(Cl)cc(-n3c(C)nc4ccc(-c5ccnc(N)c5)nc43)cc2Cl)CC1]

### 4-(3-(3,5-dichloro-4-(4-(prop-2-yn-1-yl)piperazin-1-yl)phenyl)-2-methyl-3H-imidazo[4,5-b]pyridin-5-yl)pyridin-2-amine (27e)

3-bromoprop-1-yne (10.5 mg, 70.6 μmol, 1.0 eq., 80% Wt.) was added to a 5 mL MWV equipped with a magnetic stir bar containing 4-(3-(3,5-dichloro-4-(piperazin-1-yl)phenyl)-2-methyl-3H-imidazo[4,5-b]pyridin-5-yl)pyridin-2-amine (27d) (32 mg, 70 μmol, 1.0 eq.) and potassium carbonate (29 mg, 0.21 mmol, 3.0 eq.) in DMF (1.00 mL). The reaction mixture was heated at 80° C. for 16 hours where it was then cooled down to room temperature and washed with saturated sodium bicarbonate (5 mL). The mixture was extracted with DCM (3×15 mL) and the organic layers were recombined, dried over sodium sulfate, and concentrated under reduced pressure. The crude residue was purified by automated flash column chromatography using a DCM/MeOH gradient to afford 4-(3-(3,5-dichloro-4-(4-(prop-2-yn-1-yl)piperazin-1-yl)phenyl)-2-methyl-3H-imidazo[4,5-b]pyridin-5-yl)pyridin-2-amine (27e) (9.2 mg, 19 μmol, 27%). LCMS (ESI) C₂₅H₂₃Cl₂N₇ requires 491.13, found 492 (M/Z+H).

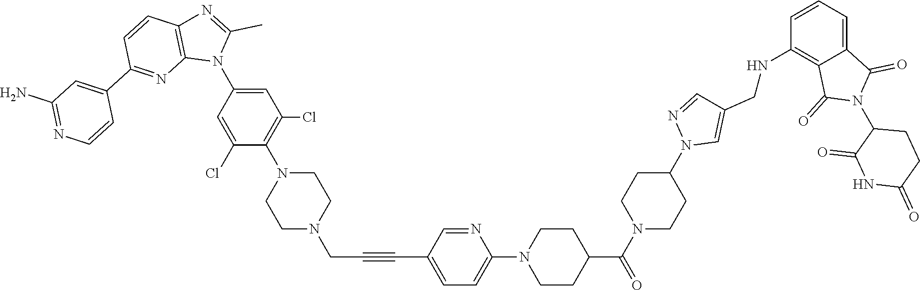 [SMILES: Cc1nc2ccc(-c3ccnc(N)c3)nc2n1-c1cc(Cl)c(N2CCN(CC#Cc3ccc(N4CCC(C(=O)N5CCC(n6cc(CNc7cccc8c7C(=O)N(C7CCC(=O)NC7=O)C8=O)cn6)CC5)CC4)nc3)CC2)c(Cl)c1]

### 4-(((1-(1-(1-(5-(3-(4-(4-(5-(2-aminopyridin-4-yl)-2-methyl-3H-imidazo[4,5-b]pyridin-3-yl)-2,6-dichlorophenyl)piperazin-1-yl)prop-1-yn-1-yl)pyridin-2-yl)piperidine-4-carbonyl)piperidin-4-yl)-1H-pyrazol-4-yl)methyl)amino)-2-(2,6-dioxopiperidin-3-yl)isoindoline-1,3-dione (27)

A flame dried 5 ml MWV equipped with a magnetic stir bar containing copper (I) iodide (0.3 mg, 1.4 μmol, 0.2 eq.), PdCl2 (dppf) (0.6 mg, 0.86 μmol, 0.12 eq.), 4-(1-(3,5-dichloro-4-(4-(prop-2-yn-1-yl)piperazin-1-yl)phenyl)-2-methyl-1H-benzo[d]imidazol-6-yl)pyridin-2-amine (27e) (3.5 mg, 7.2 μmol, 1.0 eq.), and 2-(2,6-dioxopiperidin-3-yl)-4-(((1-(1-(1-(5-iodopyridin-2-yl)piperidine-4-carbonyl)piperidin-4-yl)-1H-pyrazol-4-yl)methyl)amino)isoindoline-1,3-dione (B7) (5.4 mg, 7.2 μmol, 1.0 eq.) was backfilled with argon 3 times. DMF (0.75 mL) and triethylamine (2.2 mg, 22 μmol, 3.0 eq.) were added sequentially and the reaction mixture was stirred at 37° C. for 24 hours. The reaction mixture was concentrated under reduced pressure and purified by automated column chromatography to give 4-(((1-(1-(1-(5-(3-(4-(4-(5-(2-aminopyridin-4-yl)-2-methyl-3H-imidazo[4,5-b]pyridin-3-yl)-2,6-dichlorophenyl)piperazin-1-yl)prop-1-yn-1-yl)pyridin-2-yl)piperidine-4-carbonyl)piperidin-4-yl)-1H-pyrazol-4-yl)methyl)amino)-2-(2,6-dioxopiperidin-3-yl)isoindoline-1,3-dione (27) (3.1 mg, 2.8 μmol, 39%), a yellow solid. LCMS (ESI) C₅₈H₅₇N₁₅O₅ requires 1115.09 found 558.58 (M/2).

### Example 28. Synthesis of 4-(((1-(1-(1-(4-(3-(4-(4-(5-(2-aminopyridin-4-yl)-2-methyl-3H-imidazo[4,5-b]pyridin-3-yl)-2-fluorophenyl)piperazin-1-yl)prop-1-yn-1-yl)phenyl)cyclobutane-1-carbonyl)piperidin-4-yl)-1H-pyrazol-4-yl)methyl)amino)-2-(2,6-dioxopiperidin-3-yl)isoindoline-1,3-dione (28)

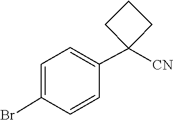 [SMILES: N#CC1(c2ccc(Br)cc2)CCC1]

### 1-(4-bromophenyl)cyclobutane-1-carbonitrile (28a)

1,3-dibromopropane (1.08 mL, 10.71 mmol, 1.05 eq.) and 2-(4-bromophenyl)acetonitrile (2.00 g, 10.20 mmol, 1.0 eq.) were added to a 250 mL RBF equipped with a magnetic stir bar containing tetrabutylammonium bromide (328 mg, 1.02 mmol, 0.1 eq.), potassium hydroxide (3.43 g, 61.20 mmol, 1.0 eq.), toluene (33.00 mL), and water (2.00 mL). The reaction mixture was stirred at 100 C for 3 hours. The reaction mixture was acidified to pH 7 and was extracted with ethyl acetate (3×15 mL). The organic phase was washed with brine, dried over sodium sulfate, and concentrated under reduced pressure. The crude residue was purified by flash column chromatography to give 1-(4-bromophenyl)cyclobutane-1-carbonitrile (28a) (1.32 g, 5.60 mmol, 54% yield, eluting at 5% ethyl acetate in hexanes), yellow liquid. ¹H NMR (500 MHz, CDCl₃) δ 7.52 (d, J=8.7 Hz, 2H), 7.29 (d, J=8.7 Hz, 2H), 2.87-2.77 (m, 2H), 2.63-2.53 (m, 2H), 2.49-2.37 (m, 1H), 2.07 (dtt, J=11.5, 9.1, 4.4 Hz, 1H). ¹³C NMR (126 MHz, CDCl₃) δ 138.96, 132.19, 127.49, 123.98, 122.04, 39.91, 34.75, 17.14.

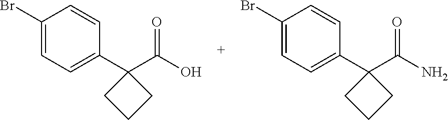 [SMILES: O=C(O)C1(c2ccc(Br)cc2)CCC1 ; NC(=O)C1(c2ccc(Br)cc2)CCC1]

### 1-(4-bromophenyl)cyclobutane-1-carboxylic acid (28b) δ 1-(4-bromophenyl)cyclobutane-1-carboxamide

To a solution of 1-(4-bromophenyl)cyclobutane-1-carbonitrile (28a) (966 mg, 1 Eq, 4.09 mmol) in ethanol (25.5 mL) and water (1.82 mL) was added potassium hydroxide (2.30 g, 40.9 mmol, 10 eq.). The reaction mixture was heated at 80 C for 16 hours where the reaction was then quenched with 1M HCl until pH 7 was reached. The ethanol was removed under reduced pressure and the residue was dissolved in ethyl acetate (40 mL) and washed with brine (3×10 mL). The organic phase was concentrated under reduced pressure and purified by flash column chromatography using to give 1-(4-bromophenyl)cyclobutane-1-carboxylic acid (207 mg, 0.81 mmol, 19% yield), a yellow crystalline solid and 1-(4-bromophenyl)cyclobutane-1-carboxamide (28b) (636 mg, 2.50 mmol, 61% yield), a orange oil. Carboxylic acid: ¹H NMR (500 MHz, CDCl₃) δ 11.65 (s, 1H), 7.46 (d, J=7.8 Hz, 2H), 7.18 (d, J=7.8 Hz, 2H), 2.89-2.80 (m, 2H), 2.49 (t, J=8.9 Hz, 2H), 2.10 (dq, J=11.6, 8.5 Hz, 1H), 1.88 (ddq, J=11.9, 9.4, 4.8 Hz, 1H). ¹³C NMR (126 MHz, CDCl₃) δ 181.99, 142.25, 131.57, 128.42, 121.07, 51.94, 32.37, 16.71. Formamide: ¹H NMR (500 MHz, CDCl₃) δ 7.53-7.46 (m, 2H), 7.22-7.16 (m, 2H), 5.60 (bs, 1H), 5.18 (bs, 1H), 3.48 (d, J=2.4 Hz, 1H), 2.86-2.77 (m, 2H), 2.48-2.38 (m, 2H), 2.14 (dtt, J=11.5, 9.2, 7.2 Hz, 1H), 1.88 (dtt, J=11.3, 9.2, 5.7 Hz, 1H). ¹³C NMR (126 MHz, CDCl₃) δ 178.09, 143.55, 132.08, 128.12, 121.07, 77.42, 77.16, 76.91, 50.96, 32.26, 16.58.

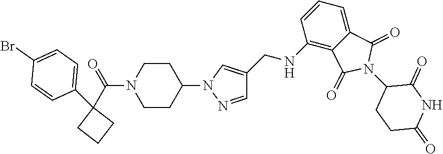 [SMILES: O=C1CCC(N2C(=O)c3cccc(NCc4cnn(C5CCN(C(=O)C6(c7ccc(Br)cc7)CCC6)CC5)c4)c3C2=O)C(=O)N1]

### 4-(((1-(1-(1-(4-bromophenyl)cyclobutane-1-carbonyl)piperidin-4-yl)-1H-pyrazol-4-yl)methyl)amino)-2-(2,6-dioxopiperidin-3-yl)isoindoline-1,3-dione (28c)

DMF (7.50 mL) and DIPEA (0.08 mL, 0.45 mmol, 6.0 eq.) were added sequentially to a 20 mL MWV equipped with a magnetic stir bar containing 2-(2,6-dioxopiperidin-3-yl)-4-(((1-(piperidin-4-yl)-1H-pyrazol-4-yl)methyl)amino)isoindoline-1,3-dione, Trifluoroacetic acid (28b) (55 mg, 75 μmol, 1.0 eq.), 1-(4-bromophenyl)cyclobutane-1-carboxylic acid (19 mg, 75 μmol, 1.0 eq.), and HATU (34 mg, 90 μmol, 1.2 eq.). The reaction mixture was stirred at room temperature for 16 hours before it was diluted with water and extracted with DCM. The organic layer was washed with brine (4×), dried over sodium sulfate, and concentrated under reduced pressure. The crude residue was purified by flash column chromatography to give 4-(((1-(1-(1-(4-bromophenyl)cyclobutane-1-carbonyl)piperidin-4-yl)-1H-pyrazol-4-yl)methyl)amino)-2-(2,6-dioxopiperidin-3-yl)isoindoline-1,3-dione (28c) (31 mg, 46 μmol, 63%, eluting at 3% MeOH in DCM), a yellow solid. ¹H NMR (500 MHz, CDCl₃) δ 7.33-7.20 (m, 4H), 7.12-7.04 (m, 2H), 7.04-6.96 (m, 2H), 6.88 (dd, J=7.2, 3.2 Hz, 1H), 6.67 (dd, J=8.5, 3.3 Hz, 1H), 4.70-4.62 (m, 1H), 4.52-4.41 (m, 1H), 4.09 (s, 2H), 3.99-3.88 (m, 1H), 3.23-3.11 (m, 1H), 2.71-2.42 (m, 5H), 2.35 (s, 4H), 2.09 (bs, 2H), 1.97-1.81 (m, 2H), 1.81-1.62 (m, 2H), 1.56 (bs, 2H). ¹³C NMR (126 MHz, CDCl₃) δ 173.88, 171.75, 169.43, 168.83, 167.69, 146.23, 142.25, 136.19, 132.43, 132.08, 126.74, 120.47, 116.83, 111.96, 110.36, 77.34, 77.29, 77.09, 76.83, 58.76, 51.96, 49.89, 49.72, 49.55, 49.38, 49.20, 49.03, 48.90, 44.12, 41.32, 37.47, 31.78, 31.38, 29.65, 22.74, 15.22. LCMS (ESI) C₃₃H₃₃BrN₆O₅ 672.16, found 673 (M/Z+H).

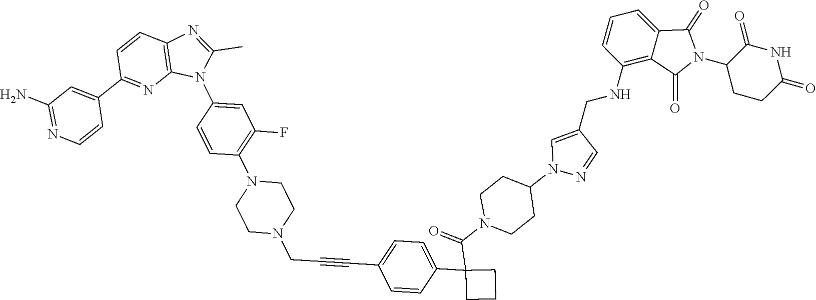 [SMILES: Cc1nc2ccc(-c3ccnc(N)c3)nc2n1-c1ccc(N2CCN(CC#Cc3ccc(C4(C(=O)N5CCC(n6cc(CNc7cccc8c7C(=O)N(C7CCC(=O)NC7=O)C8=O)cn6)CC5)CCC4)cc3)CC2)c(F)c1]

### 4-(((1-(1-(1-(4-(3-(4-(4-(5-(2-aminopyridin-4-yl)-2-methyl-3H-imidazo[4,5-b]pyridin-3-yl)-2-fluorophenyl)piperazin-1-yl)prop-1-yn-1-yl)phenyl)cyclobutane-1-carbonyl)piperidin-4-yl)-1H-pyrazol-4-yl)methyl)amino)-2-(2,6-dioxopiperidin-3-yl)isoindoline-1,3-dione (28)

A flame dried 5 ml MWV equipped with a magnetic stir bar containing PdCl₂(PPh₃)₂ (4 mg, 5.71 μmol, 0.12 eq.), copper (I) iodide (2.0 mg, 0.01 mmol, 0.2 eq.), 4-(3-(3-fluoro-4-(4-(prop-2-yn-1-yl)piperazin-1-yl)phenyl)-2-methyl-3H-imidazo[4,5-b]pyridin-5-yl)pyridin-2-amine (A8) (21 mg, 47 μmol, 1.0 eq.), and 4-(((1-(1-(1-(4-bromophenyl)cyclobutane-1-carbonyl)piperidin-4-yl)-1H-pyrazol-4-yl)methyl)amino)-2-(2,6-dioxopiperidin-3-yl)isoindoline-1,3-dione (B7) (32 mg, 47.6 μmol, 1.0 eq.) was backfilled with argon 3 times. DMF (0.80 mL) and triethylamine (19 mg, 190 μmol, 4,0 eq.) were added sequentially, and the reaction mixture was heated at 40° C. for 16 hours). The reaction mixture was concentrated under reduced pressure and purified by automated column chromatography to give 4-(((1-(1-(1-(4-(3-(4-(4-(5-(2-aminopyridin-4-yl)-2-methyl-3H-imidazo[4,5-b]pyridin-3-yl)-2-fluorophenyl)piperazin-1-yl)prop-1-yn-1-yl)phenyl)cyclobutane-1-carbonyl)piperidin-4-yl)-1H-pyrazol-4-yl)methyl)amino)-2-(2,6-dioxopiperidin-3-yl)isoindoline-1,3-dione (28) (3 mg, 3 μmol, 5%, 88% purity), a yellow solid. LCMS (ESI) C₅₈H₅₆FN₁₃₀₅ requires 1033.45, found 1034 (M/Z+H).

### Examples 29 and 30. Synthesis of (R)-4-(((1-(1-(1-(5-(3-(4-(4-(5-(2-aminopyridin-4-yl)-2-methyl-3H-imidazo[4,5-b]pyridin-3-yl)-2-fluorophenyl)piperazin-1-yl)prop-1-yn-1-yl)pyridin-2-yl)piperidine-4-carbonyl)piperidin-4-yl)-1H-pyrazol-4-yl)methyl)amino)-2-(2,6-dioxopiperidin-3-yl)isoindoline-1,3-dione (29)

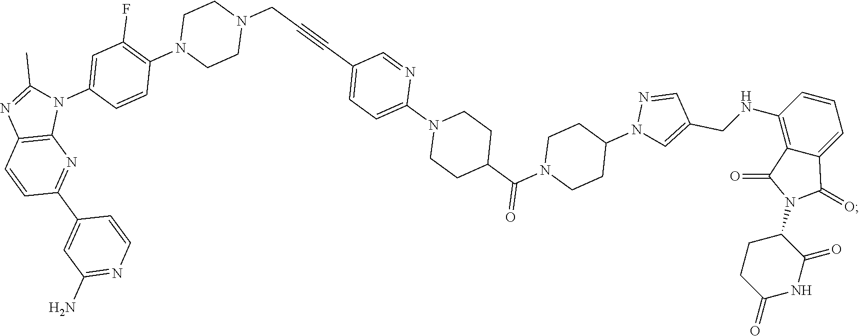 [SMILES: Cc1nc2ccc(-c3ccnc(N)c3)nc2n1-c1ccc(N2CCN(CC#Cc3ccc(N4CCC(C(=O)N5CCC(n6cc(CNc7cccc8c7C(=O)N([C@H]7CCC(=O)NC7=O)C8=O)cn6)CC5)CC4)nc3)CC2)c(F)c1]

and(S)-4-(((1-(1-(1-(5-(3-(4-(4-(5-(2-aminopyridin-4-yl)-2-methyl-3H-imidazo[4,5-b]pyridin-3-yl)-2-fluorophenyl)piperazin-1-yl)prop-1-yn-1-yl)pyridin-2-yl)piperidine-4-carbonyl)piperidin-4-yl)-1H-pyrazol-4-yl)methyl)amino)-2-(2,6-dioxopiperidin-3-yl)isoindoline-1,3-dione (30)

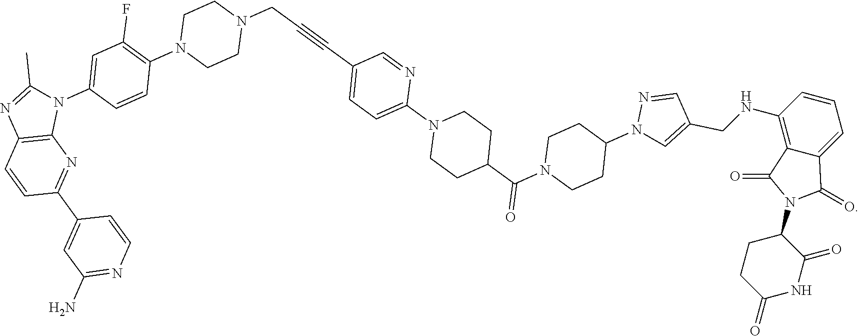 [SMILES: Cc1nc2ccc(-c3ccnc(N)c3)nc2n1-c1ccc(N2CCN(CC#Cc3ccc(N4CCC(C(=O)N5CCC(n6cc(CNc7cccc8c7C(=O)N([C@@H]7CCC(=O)NC7=O)C8=O)cn6)CC5)CC4)nc3)CC2)c(F)c1]

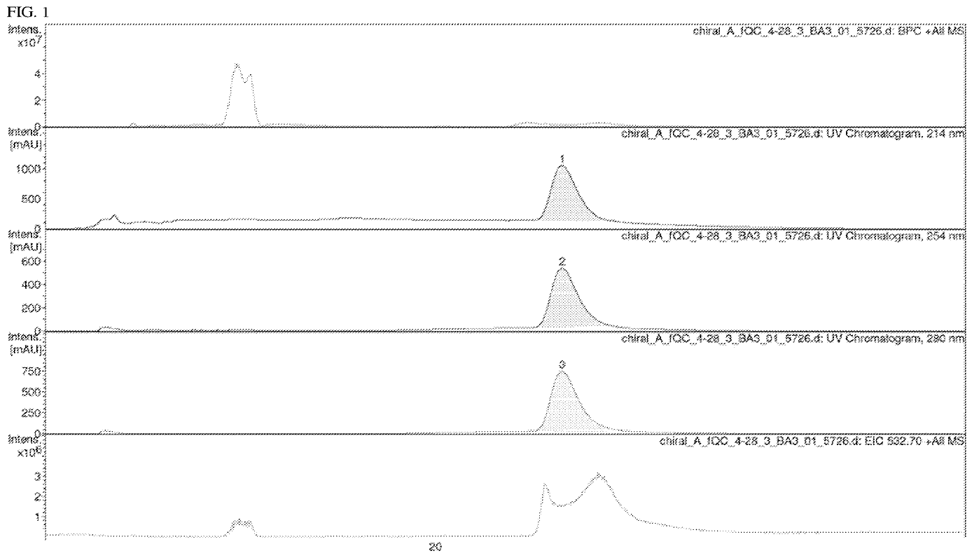

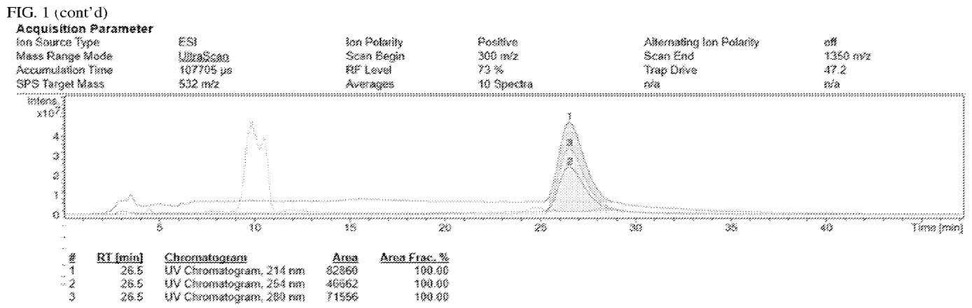

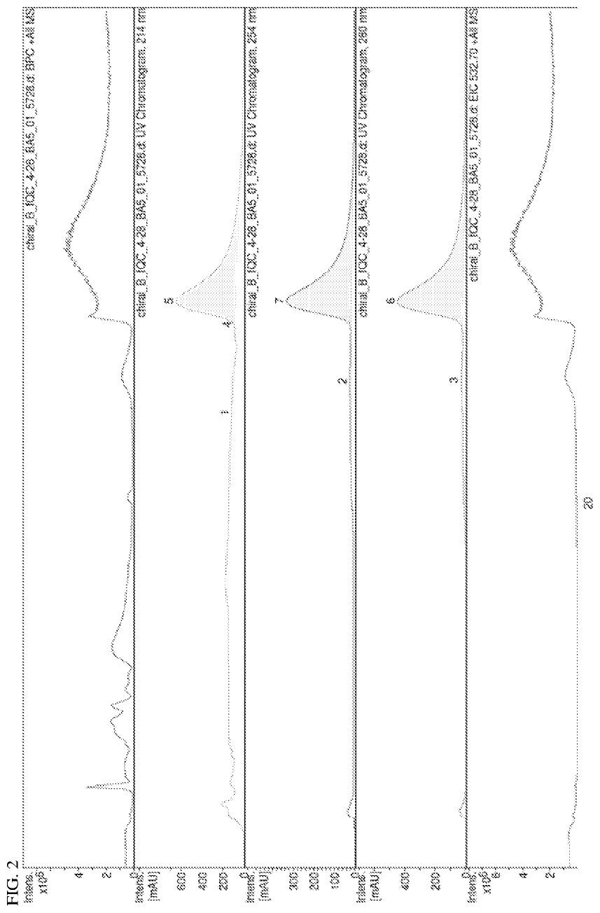

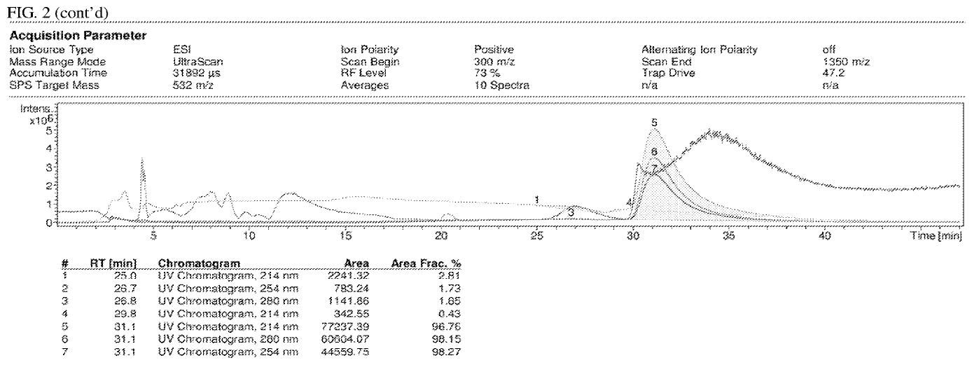

Chiral Chromatography. Using a Daicel Chiraltek AD-RH column 18 (40 mg, 37.5 μmol) was separated into its two enantiomers using an eluent system consisting of solvent A: 50/50 MeCN/water, 5 mM ammonium formate, 1 mL formic acid and solvent B: 88/10/2 IPA/MeCN/water, 5 mM ammonium formate, 1 mL formic acid. (29) (11.88 mg, 11.1 μmol, 29% recovery, >99% ee) was isolated as a yellow solid and DYR874 (30) (10.92 mg, 10.2 μM, 27% recovery, 97.7% ee), a yellow solid were isolated and eluted in this order. Chiral HPLC of 29 is shown at FIG. 1. Chiral HPLC of 30 is shown at FIG. 2.

### Example 31. Synthesis of 4-(((1-(1-(1-(5-(3-(4-(4-(5-(2-aminopyridin-4-yl)-2-methyl-3H-imidazo[4,5-b]pyridin-3-yl)-2-fluorophenyl)piperazin-1-yl)prop-1-yn-1-yl)pyridin-2-yl)piperidine-4-carbonyl)piperidin-4-yl)-1H-pyrazol-4-yl)methyl)amino)-2-(1-methyl-2,6-dioxopiperidin-3-yl)isoindoline-1,3-dione (31)

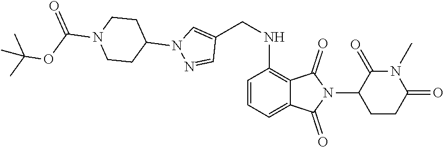 [SMILES: CN1C(=O)CCC(N2C(=O)c3cccc(NCc4cnn(C5CCN(C(=O)OC(C)(C)C)CC5)c4)c3C2=O)C1=O]

### tert-butyl 4-(4-(((2-(1-methyl-2,6-dioxopiperidin-3-yl)-1,3-dioxoisoindolin-4-yl)amino)methyl)-1H-pyrazol-1-yl)piperidine-1-carboxylate (31a)

To a dry 5 mL MWV equipped with a magnetic stir bar containing tert-butyl 4-(4-formyl-1H-pyrazol-1-yl)piperidine-1-carboxylate (16b) (57 mg, 0.20 mmol, 1.0 eq.), 4-amino-2-(1-methyl-2,6-dioxopiperidin-3-yl)isoindoline-1,3-dione (59 mg, 0.20 mmol, 1.0 eq.), and dibutyldichlorostannane (62 mg, 0.20 mmol) was added DMF (0.82 mmol) and phenylsilane (22 mg, 0.20 mmol, 1.0 eq.). The reaction mixture was heated at 80° C. for 24 hours where it was then cooled down to room temperature and concentrated under reduced pressure. The crude residue was purified by flash column chromatography to give tert-butyl 4-(4-(((2-(1-methyl-2,6-dioxopiperidin-3-yl)-1,3-dioxoisoindolin-4-yl)amino)methyl)-1H-pyrazol-1-yl)piperidine-1-carboxylate (31a) (118 mg, 0.20 mmol, 98% yield, 93% pure), a yellow solid. ¹H NMR (400 MHz, CDCl₃) δ 8.01 (s, 1H), 7.55-7.49 (m, 2H), 7.19-7.14 (m, 1H), 6.91-6.84 (m, 1H), 4.91 (dd, J=12.1, 5.7 Hz, 1H), 4.64 (s, 1H), 4.42 (s, 2H), 4.26 (d, J=13.8 Hz, 3H), 3.05-2.89 (m, 1H), 2.88 (s, 3H), 2.82-2.66 (m, 2H), 2.22 (s, 2H), 2.10 (s, 1H), 1.96-1.78 (m, 3H), 1.45 (s, 9H), 0.98-0.90 (m, 1H).

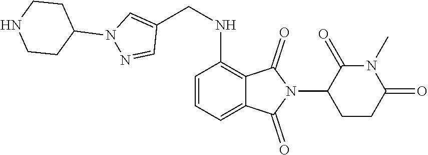 [SMILES: CN1C(=O)CCC(N2C(=O)c3cccc(NCc4cnn(C5CCNCC5)c4)c3C2=O)C1=O]

### 2-(1-methyl-2,6-dioxopiperidin-3-yl)-4-(((1-(piperidin-4-yl)-1H-pyrazol-4-yl)methyl)amino)isoindoline-1,3-dione (31b)

Trifluoroacetic acid (0.31 mL, 3.99 mmol, 20.0 eq.) was added to a 5 mL MWV equipped with a magnetic stir bar containing tert-butyl 4-(4-(((2-(1-methyl-2,6-dioxopiperidin-3-yl)-1,3-dioxoisoindolin-4-yl)amino)methyl)-1H-pyrazol-1-yl)piperidine-1-carboxylate (31a) (118 mg, 0.20 mmol, 1.0 eq.) and DCM (1.50 mL). The reaction mixture was stirred at room temperature for 24 hours to give 2-(1-methyl-2,6-dioxopiperidin-3-yl)-4-(((1-(piperidin-4-yl)-1H-pyrazol-4-yl)methyl)amino)isoindoline-1,3-dione, Trifluoroacetic acid (31b), a yellow semisolid and was used in the next step without further purification. LCMS (ESI) C₂₃H₂₆N₆O₄ requires 450.20, found 451 (M/Z+H).

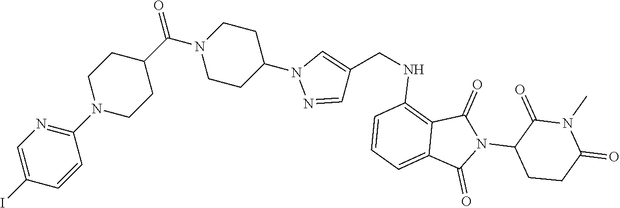 [SMILES: CN1C(=O)CCC(N2C(=O)c3cccc(NCc4cnn(C5CCN(C(=O)C6CCN(c7ccc(I)cn7)CC6)CC5)c4)c3C2=O)C1=O]

### 4-(((1-(1-(1-(5-iodopyridin-2-yl)piperidine-4-carbonyl)piperidin-4-yl)-1H-pyrazol-4-yl)methyl)amino)-2-(1-methyl-2,6-dioxopiperidin-3-yl)isoindoline-1,3-dione (31c)

DMF (1.60 mL) and DIPEA (0.16 mL, 0.96 mmol, 6.0 eq.) were added sequentially to a reaction vessel containing HATU (67 mg, 0.17 mmol, 1.1 eq.), 2-(1-methyl-2,6-dioxopiperidin-3-yl)-4-(((1-(piperidin-4-yl)-1H-pyrazol-4-yl)methyl)amino)isoindoline-1,3-dione, Trifluoroacetic acid (31b) (113 mg, 0.16 mmol, 80% pure), and 1-(5-iodopyridin-2-yl)piperidine-4-carboxylic acid (58 mg, 0.17 mmol, 1.1 eq.). The resulting solution was stirred at room temperature for 16 hours where the reaction mixture was then diluted with water and extracted with DCM. The organic layer was washed with brine (4×), dried over sodium sulfate, and concentrated under reduced pressure. The crude residue was triturated with heptanes and concentrated under reduced pressure (5×) to remove the residual DMF. The crude residue was the purified by flash column chromatography to give 4-(((1-(1-(1-(5-iodopyridin-2-yl)piperidine-4-carbonyl)piperidin-4-yl)-1H-pyrazol-4-yl)methyl)amino)-2-(1-methyl-2,6-dioxopiperidin-3-yl)isoindoline-1,3-dione (31c) (62 mg, 0.08 mmol, 51% yield, eluting at 4% MeOH in DCM), a yellow solid. ¹H NMR (500 MHz, CDCl₃) δ 8.25 (d, J=2.3 Hz, 1H), 7.67 (ddd, J=8.3, 5.1, 3.0 Hz, 1H), 7.53-7.46 (m, 2H), 7.42 (s, 1H), 7.12 (d, J=7.2 Hz, 1H), 6.93 (d, J=8.5 Hz, 1H), 6.59-6.52 (m, 1H), 6.41 (t, J=5.6 Hz, 1H), 4.90 (q, J=5.4 Hz, 1H), 4.71 (d, J=13.8 Hz, 1H), 4.38-4.28 (m, 3H), 4.25 (d, J=13.3 Hz, 2H), 4.08 (d, J=13.9 Hz, 1H), 3.20 (s, 3H), 3.16 (dtd, J=7.5, 4.4, 2.2 Hz, 2H), 3.02-2.91 (m, 2H), 2.87 (s, 1H), 2.83-2.71 (m, 3H), 2.24 (d, J=12.8 Hz, 1H), 2.18-2.05 (m, 2H), 2.03-1.74 (m, 6H). LCMS (ESI) C₃₄H₃₇IN₈O₈ requires 764.19, found 765 (M/Z+H).

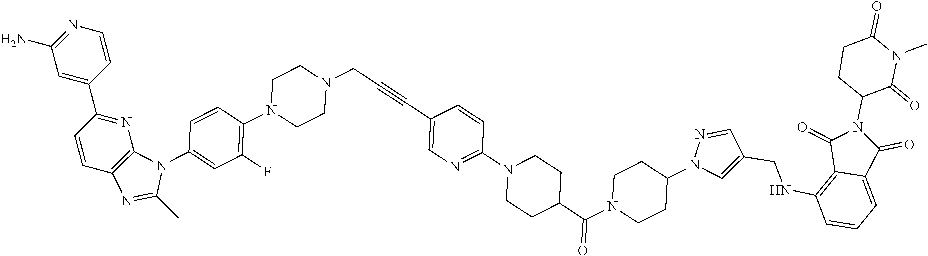 [SMILES: Cc1nc2ccc(-c3ccnc(N)c3)nc2n1-c1ccc(N2CCN(CC#Cc3ccc(N4CCC(C(=O)N5CCC(n6cc(CNc7cccc8c7C(=O)N(C7CCC(=O)N(C)C7=O)C8=O)cn6)CC5)CC4)nc3)CC2)c(F)c1]

4-(((1-(1-(1-(5-(3-(4-(4-(5-(2-aminopyridin-4-yl)-2-methyl-3H-imidazo[4,5-b]pyridin-3-yl)-2-fluorophenyl)piperazin-1-yl)prop-1-yn-1-yl)pyridin-2-yl)piperidine-4-carbonyl)piperidin-4-yl)-1H-pyrazol-4-yl)methyl)amino)-2-(1-methyl-2,6-dioxopiperidin-3-yl)isoindoline-1,3-dione (31) To a dry 5 mL MWV equipped with a magnetic stir bar was added 4-(3-(3-fluoro-4-(4-(prop-2-yn-1-yl)piperazin-1-yl)phenyl)-2-methyl-3H-imidazo[4,5-b]pyridin-5-yl)pyridin-2-amine (A10) (25 mg, 58 μmol, 1.0 eq.), 4-(((1-(1-(1-(5-iodopyridin-2-yl)piperidine-4-carbonyl)piperidin-4-yl)-1H-pyrazol-4-yl)methyl)amino)-2-(1-methyl-2,6-dioxopiperidin-3-yl)isoindoline-1,3-dione (31) (44 mg, 58 μmol, 1.0 eq.), PdCl₂ (dppf) (5.5 mg, 7.5 μmol, 0.13 eq.), and copper (I) iodide (2.7 mg, 14 μmol, 0.25 eq.). The microwave vial was sealed and put under a nitrogen atmosphere where DMF (1.0 mL) and triethylamine (29 mg, 0.29 mmol, 5.0 eq.) were added sequentially. The reaction mixture was stirred at 24 hours at room temperature where it was concentrated under reduced pressure and purified by flash column chromatography to give 4-(((1-(1-(1-(5-(3-(4-(4-(5-(2-aminopyridin-4-yl)-2-methyl-3H-imidazo[4,5-b]pyridin-3-yl)-2-fluorophenyl)piperazin-1-yl)prop-1-yn-1-yl)pyridin-2-yl)piperidine-4-carbonyl)piperidin-4-yl)-1H-pyrazol-4-yl)methyl)amino)-2-(1-methyl-2,6-dioxopiperidin-3-yl)isoindoline-1,3-dione (31), a yellow solid. ¹H NMR (400 MHz, CDCl₃) δ 8.27 (d, J=2.3 Hz, 1H), 8.06 (d, J=8.3 Hz, 1H), 7.95 (d, J=5.7 Hz, 1H), 7.75 (d, J=8.3 Hz, 1H), 7.54-7.46 (m, 3H), 7.41 (s, 1H), 7.29 (d, J=6.0 Hz, 2H), 7.24-7.08 (m, 4H), 6.92 (d, J=8.5 Hz, 1H), 6.59 (d, J=8.9 Hz, 1H), 6.42 (t, J=5.6 Hz, 1H), 5.73 (s, 2H), 4.97-4.86 (m, 1H), 4.74 (d, J=13.5 Hz, 1H), 4.39-4.26 (m, 5H), 4.15-4.02 (m, 1H), 3.62 (s, 2H), 3.38-3.21 (m, 5H), 3.03-2.85 (m, 8H), 2.85-2.69 (m, 4H), 2.60 (s, 3H), 2.31-2.05 (m, 4H), 2.02-1.75 (m, 7H). ¹⁹F NMR (376 MHz, CDCl₃) δ -118.96 (dd, J=12.7, 7.7 Hz).

### Example 32. Synthesis of 4-(((1-(1-(1-(6-(3-(4-(4-(5-(2-aminopyridin-4-yl)-2-methyl-3H-imidazo[4,5-b]pyridin-3-yl)-2-fluorophenyl)piperazin-1-yl)prop-1-yn-1-yl)pyrazin-2-yl)piperidine-4-carbonyl)piperidin-4-yl)-1H-pyrazol-4-yl)methyl)amino)-2-(2,6-dioxopiperidin-3-yl)isoindoline-1,3-dione (32)

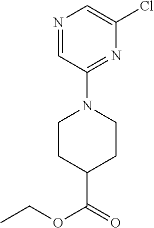 [SMILES: CCOC(=O)C1CCN(c2cncc(Cl)n2)CC1]

### ethyl 1-(6-chloropyrazin-2-yl)piperidine-4-carboxylate (32a)

Triethylamine (1.03 mL, 7.384 mmol, 1.1 eq.) and ethyl piperidine-4-carboxylate (1.09 mL, 7.04 mmol, 1.05 eq.) were added to a 100 mL RBF containing 2,6-dichloropyrazine (1.00 g, 6.71 mmol, 1.0 eq.) and 1,4-dioxane (34.00 mL). The reaction mixture was stirred at 100° C. for 16 hours where the reaction mixture was concentrated under reduced pressure and purified by automated column chromatography to give ethyl 1-(6-chloropyrazin-2-yl)piperidine-4-carboxylate (34a) (1.37 g, 5.08 mmol, 75% yield), a red oil. ¹H NMR (400 MHz, CDCl₃) δ 7.97 (d, J=0.6 Hz, 1H), 7.77 (s, 1H), 4.26-4.18 (m, 1H), 4.15 (q, J=7.1 Hz, 2H), 3.07 (ddd, J=13.5, 11.3, 3.0 Hz, 2H), 2.57 (tt, J=10.8, 4.0 Hz, 1H), 2.06-1.95 (m, 2H), 1.75 (dtd, J=13.6, 11.0, 4.0 Hz, 2H), 1.25 (t, J=7.1 Hz, 3H). ¹³C NMR (101 MHz, CDCl₃) δ 174.30, 154.12, 146.70, 130.51, 128.05, 60.76, 44.13, 41.00, 27.58, 14.32.

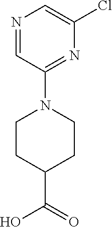 [SMILES: O=C(O)C1CCN(c2cncc(Cl)n2)CC1]

### 1-(6-chloropyrazin-2-yl)piperidine-4-carboxylic acid (32b)

To a solution of ethyl 1-(6-chloropyrazin-2-yl)piperidine-4-carboxylate (32a) (1.28 g, 4.76 mmol, 1.0 eq.) in THF (56.5 mL) and Methanol (18.8 mL) was added lithium hydroxide, water (400 mg, 9.53 mmol, 2.0 eq.). The resulting mixture was stirred at room temperature for 16 hours. Water (50 mL) was added, and the reaction mixture was concentrated under reduced pressure. Water (50 ml) and ether (100 mL) were added and the solution was stirred for 10 minutes. The ethereal layer was removed, and the aqueous layer acidified to pH 2 with 1N HCl at 0° C. and stirred for 20 minutes. The white precipitate was collected by filtration and washed with ice-cold water to give 1-(6-chloropyrazin-2-yl)piperidine-4-carboxylic acid (32b) (809 mg, 3.35 mmol, 70% yield). LCMS (ESI) C₁₀H₁₂ClN₃O₂ requires 241.06, found 242.2 (M/Z+H).

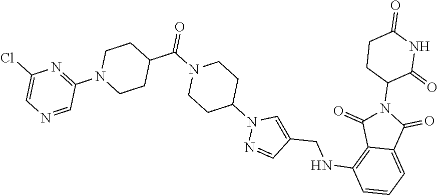 [SMILES: O=C1CCC(N2C(=O)c3cccc(NCc4cnn(C5CCN(C(=O)C6CCN(c7cncc(Cl)n7)CC6)CC5)c4)c3C2=O)C(=O)N1]

### 4-(((1-(1-(1-(6-chloropyrazin-2-yl)piperidine-4-carbonyl)piperidin-4-yl)-1H-pyrazol-4-yl)methyl)amino)-2-(2,6-dioxopiperidin-3-yl)isoindoline-1,3-dione (32c)

DMF (2.7 mL) and DIPEA (0.28 mL, 1.63 mmol, 6. 0 eq.) were added sequentially to a reaction vessel containing HATU (124 mg, 0.32 mmol, 1.2 eq.), 2-(2,6-dioxopiperidin-3-yl)-4-(((1-(piperidin-4-yl)-1H-pyrazol-4-yl)methyl)amino)isoindoline-1,3-dione, trifluoroacetic acid (16d) (200 mg, 0.27 mmol, 1.0 eq., 75% Wt.), and 1-(6-chloropyrazin-2-yl)piperidine-4-carboxylic acid (32b) (72 mg, 0.30 mmol, 1.1 eq.). The resulting solution was stirred at room temperature for 16 hours before the reaction mixture was diluted with water and extracted with ethyl acetate. The organic layer was washed with brine (4×), dried over sodium sulfate, and concentrated under reduced pressure. The crude residue was purified by flash column chromatography to give 4-(((1-(1-(1-(6-chloropyrazin-2-yl)piperidine-4-carbonyl)piperidin-4-yl)-1H-pyrazol-4-yl)methyl)amino)-2-(2,6-dioxopiperidin-3-yl)isoindoline-1,3-dione (32c) (126 mg, 0.19 mmol, 70% yield), a yellow solid. ¹H NMR (400 MHz, CDCl₃) δ 8.33 (s, 1H), 7.99 (s, 1H), 7.78 (s, 1H), 7.54-7.46 (m, 2H), 7.41 (d, J=0.8 Hz, 1H), 7.13 (d, J=7.1 Hz, 1H), 6.93 (d, J=8.5 Hz, 1H), 6.42 (s, 1H), 4.91 (dd, J=12.1, 5.3 Hz, 1H), 4.73 (d, J=13.5 Hz, 1H), 4.42-4.28 (m, 4H), 4.06 (d, J=13.8 Hz, 1H), 3.25 (t, J=13.0 Hz, 1H), 3.10-2.97 (m, 2H), 2.93-2.66 (m, 4H), 2.26 (d, J=12.9 Hz, 1H), 2.21-2.11 (m, 1H), 1.98-1.62 (m, 8H), 1.58-1.38 (m, 1H). LCMS (ESI) C₃₂H₃₄ClN₉O₈ requires 659.23, found 660.3 (M/Z+H).

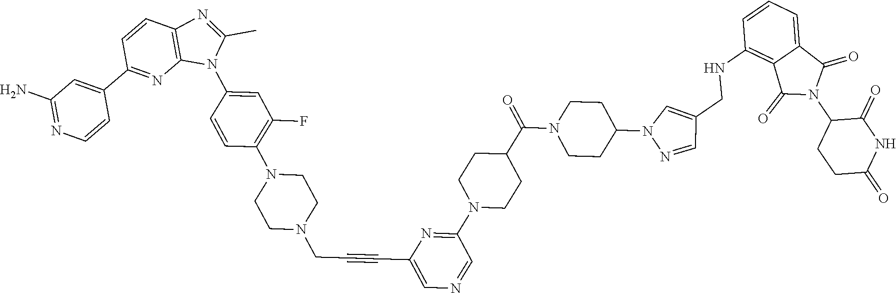 [SMILES: Cc1nc2ccc(-c3ccnc(N)c3)nc2n1-c1ccc(N2CCN(CC#Cc3cncc(N4CCC(C(=O)N5CCC(n6cc(CNc7cccc8c7C(=O)N(C7CCC(=O)NC7=O)C8=O)cn6)CC5)CC4)n3)CC2)c(F)c1]

### 4-(((1-(1-(1-(6-(3-(4-(4-(5-(2-aminopyridin-4-yl)-2-methyl-3H-imidazo[4,5-b]pyridin-3-yl)-2-fluorophenyl)piperazin-1-yl)prop-1-yn-1-yl)pyrazin-2-yl)piperidine-4-carbonyl)piperidin-4-yl)-1H-pyrazol-4-yl)methyl)amino)-2-(2,6-dioxopiperidin-3-yl)isoindoline-1,3-dione (32)

DMF (0.64 mL) and triethylamine (8.4 mg, 83 μmol, 5.0 eq.) were added sequentially to a dry 5 mL MWV equipped with a magnetic stir bar containing 4-(3-(3-fluoro-4-(4-(prop-2-yn-1-yl)piperazin-1-yl)phenyl)-2-methyl-3H-imidazo[4,5-b]pyridin-5-yl)pyridin-2-amine (A10) (7.4 mg, 17 μmol, 1.0 eq.), 4-(((1-(1-(1-(6-chloropyrazin-2-yl)piperidine-4-carbonyl)piperidin-4-yl)-1H-pyrazol-4-yl)methyl)amino)-2-(2,6-dioxopiperidin-3-yl)isoindoline-1,3-dione (32c) (11 mg, 17 μmol, 1.0 eq.), PdCl₂ (dppf) (1.6 mg, 2.2 μmol, 0.13 eq.) and copper (I) iodide (0.8 mg, 4.2 μmol, 0.25 eq.). This reaction mixture was stirred at room temperature for 24 hours where it was concentrated under reduced pressure and purified by flash column chromatography to give 4-(((1-(1-(1-(6-(3-(4-(4-(5-(2-aminopyridin-4-yl)-2-methyl-3H-imidazo[4,5-b]pyridin-3-yl)-2-fluorophenyl)piperazin-1-yl)prop-1-yn-1-yl)pyrazin-2-yl)piperidine-4-carbonyl)piperidin-4-yl)-1H-pyrazol-4-yl)methyl)amino)-2-(2,6-dioxopiperidin-3-yl)isoindoline-1,3-dione (32) (7.0 mg, 6.6 μmol, 40%), a yellow solid. LCMS (ESI) C₅₇H₅₇FN 1605 requires 1064.46, found 1065 (M/Z +H).

### Example 33. 4-(((1-(1-(1-(5-(3-(4-(4-(5-(2-aminopyridin-4-yl)-2-methyl-3H-imidazo[4,5-b]pyridin-3-yl)-2-fluorophenyl)piperazin-1-yl)prop-1-yn-1-yl)pyridin-2-yl)-4-hydroxypiperidine-4-carbonyl)piperidin-4-yl)-1H-pyrazol-4-yl)methyl)amino)-2-(2,6-dioxopiperidin-3-yl)isoindoline-1,3-dione

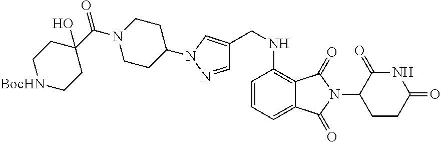

### tert-butyl 4-(4-(4-(((2-(2,6-dioxopiperidin-3-yl)-1,3-dioxoisoindolin-4-yl)amino)methyl)-1H-pyrazol-1-yl)piperidine-1-carbonyl)-4-hydroxypiperidine-1-carboxylate (33a)

DMF (0.85 mL) and DIPEA (0.07 mL, 0.43 mol, 5.0 eq.) were added sequentially to a reaction vessel containing HATU (36 mg, 94 μmol, 1.1 eq.), 2-(2,6-dioxopiperidin-3-yl)-4-(((1-(piperidin-4-yl)-1H-pyrazol-4-yl)methyl)amino)isoindoline-1,3-dione, Trifluoroacetic acid (47 mg, 85 μmol, 1.0 eq.), and 1-(tert-butoxycarbonyl)-4-hydroxypiperidine-4-carboxylic acid (21 mg, 85 μmol, 1.0 eq.). The resulting solution was stirred at room temperature for 16 hours before being diluted with water and extracted with ethyl acetate. The organic layers were washed with brine, combined, dried over sodium sulfate, and concentrated under reduced pressure. The crude residue was purified by flash column chromatography to give tert-butyl 4-(4-(4-(((2-(2,6-dioxopiperidin-3-yl)-1,3-dioxoisoindolin-4-yl)amino)methyl)-1H-pyrazol-1-yl)piperidine-1-carbonyl)-4-hydroxypiperidine-1-carboxylate (33a) (28 mg, 42 μmol, 49% yield), a yellow solid. ¹H NMR (500 MHz, CDCl₃) δ 8.63 (s, 1H), 7.53-7.45 (m, 2H), 7.41 (s, 1H), 7.12 (d, J=7.1 Hz, 1H), 6.93 (d, J=8.5 Hz, 1H), 6.41 (t, J=5.5 Hz, 1H), 5.29 (s, 1H), 4.90 (dd, J=12.1, 5.4 Hz, 1H), 4.68 (d, J=13.7 Hz, 2H), 4.38-4.28 (m, 3H), 4.00-3.79 (m, 4H), 3.71 (hept, J=6.7 Hz, 2H), 3.17 (q, J=7.4 Hz, 3H), 2.97 (d, J=11.1 Hz, 1H), 2.91-2.82 (m, 1H), 2.80-2.63 (m, 2H), 2.13 (dp, J=7.9, 5.0 Hz, 3H), 2.06-1.87 (m, 2H), 1.44 (s, 9H). ¹³C NMR (126 MHz, CDCl₃) δ 173.37, 171.37, 169.57, 168.78, 167.65, 154.97, 146.54, 138.36, 136.37, 132.58, 126.18, 118.12, 117.10, 112.16, 110.57, 79.80, 73.00, 59.07, 55.91, 49.06, 43.83, 37.79, 32.76, 31.55, 28.57, 22.90, 12.70.

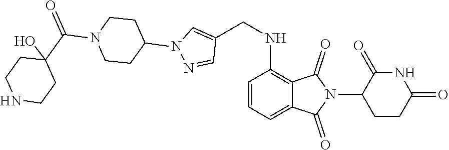 [SMILES: O=C1CCC(N2C(=O)c3cccc(NCc4cnn(C5CCN(C(=O)C6(O)CCNCC6)CC5)c4)c3C2=O)C(=O)N1]

### 2-(2,6-dioxopiperidin-3-yl)-4-(((1-(1-(4-hydroxypiperidine-4-carbonyl)piperidin-4-yl)-1H-pyrazol-4-yl)methyl)amino)isoindoline-1,3-dione (33b)

Trifluoroacetic acid (0.13 mL, 1.7 mmol, 40.0 eq.) was added to a 20 mL dram vial containing tert-butyl 4-(4-(4-(((2-(2,6-dioxopiperidin-3-yl)-1,3-dioxoisoindolin-4-yl)amino)methyl)-1H-pyrazol-1-yl)piperidine-1-carbonyl)-4-hydroxypiperidine-1-carboxylate (33a) (28 mg, 42 μmol, 1.0 eq.) in DCM (0.65 mL). The reaction mixture was stirred for 48 hours at room temperature where it was then concentrated under reduced pressure, extracted with DCM, and washed with a saturated solution of sodium bicarbonate. The organic layers were combined, dried over sodium sulfate, and concentrated under reduced pressure to give 2-(2,6-dioxopiperidin-3-yl)-4-(((1-(1-(4-hydroxypiperidine-4-carbonyl)piperidin-4-yl)-1H-pyrazol-4-yl)methyl)amino)isoindoline-1,3-dione (33b) (21 mg, 37 μmol, 88% yield), a yellow solid. ¹H NMR (500 MHz, CDCl₃) δ 7.31-7.15 (m, 3H), 6.93-6.83 (m, 1H), 6.67 (dd, J=8.9, 5.9 Hz, 1H), 4.66 (h, J=6.4 Hz, 1H), 4.57 (s, 2H), 4.19-4.00 (m, 3H), 3.09-2.68 (m, 9H), 2.68-2.33 (m, 4H), 2.20-1.80 (m, 4H), 1.80-1.53 (m, 3H), 1.46 (d, J=12.4 Hz, 1H). ¹³C NMR (126 MHz, CDCl₃) δ 172.71, 171.99, 169.53, 169.11, 167.77, 146.29, 138.25, 136.29, 132.49, 126.53, 118.07, 116.94, 112.05, 110.43, 59.16, 40.42, 37.47, 32.57, 31.44, 29.72, 22.80.

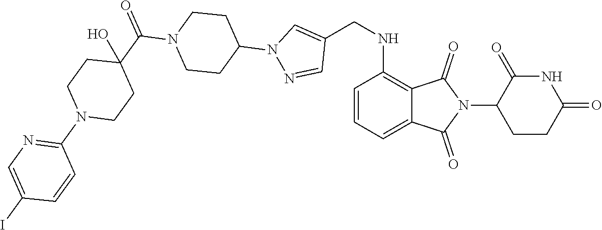 [SMILES: O=C1CCC(N2C(=O)c3cccc(NCc4cnn(C5CCN(C(=O)C6(O)CCN(c7ccc(I)cn7)CC6)CC5)c4)c3C2=O)C(=O)N1]

### 2-(2,6-dioxopiperidin-3-yl)-4-(((1-(1-(4-hydroxy-1-(5-iodopyridin-2-yl)piperidine-4-carbonyl)piperidin-4-yl)-1H-pyrazol-4-yl)methyl)amino)isoindoline-1,3-dione (33c)

To a solution of DMF (0.75 mL), potassium carbonate (13 mg, 93 μmol, 2.5 eq.), and 2-(2,6-dioxopiperidin-3-yl)-4-(((1-(1-(4-hydroxy-1-(5-iodopyridin-2-yl)piperidine-4-carbonyl)piperidin-4-yl)-1H-pyrazol-4-yl)methyl)amino)isoindoline-1,3-dione (33b) (21 mg, 37 μmol, 1.0 eq.) was added 2-fluoro-5-iodopyridine (10 mg, 45 μmol, 1.2 eq.). The MWV was capped and stirred at 110° C. for 24 hours. After stirring, the reaction mixture was cooled to room temperature, co and concentrated under reduced pressure. The crude residue was extracted with DCM and washed with distilled water (3×) and brine (1×). The combined extract(s) were dried over Na₂SO₄ and concentrated under reduced pressure. The residue was purified by flash column chromatography using a DCM/MeOH gradient to give 2-(2,6-dioxopiperidin-3-yl)-4-(((1-(1-(4-hydroxy-1-(5-iodopyridin-2-yl)piperidine-4-carbonyl)piperidin-4-yl)-1H-pyrazol-4-yl)methyl)amino)isoindoline-1,3-dione (33c) (13 mg, 17 μmol, 46% yield), a yellow solid. LCMS (ESI) C₃₃H₃₅IN₈₀₆ requires 766.17, found 767.5 (M/Z+H).

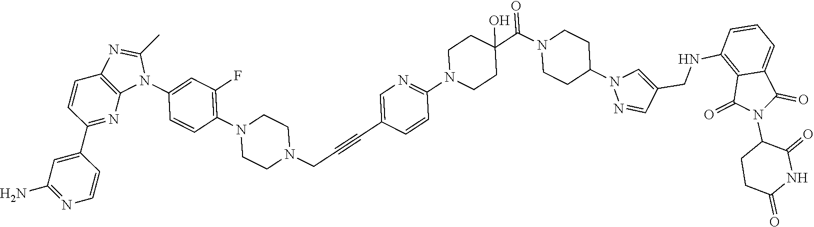 [SMILES: Cc1nc2ccc(-c3ccnc(N)c3)nc2n1-c1ccc(N2CCN(CC#Cc3ccc(N4CCC(O)(C(=O)N5CCC(n6cc(CNc7cccc8c7C(=O)N(C7CCC(=O)NC7=O)C8=O)cn6)CC5)CC4)nc3)CC2)c(F)c1]

### 4-(((1-(1-(1-(5-(3-(4-(4-(5-(2-aminopyridin-4-yl)-2-methyl-3H-imidazo[4,5-b]pyridin-3-yl)-2-fluorophenyl)piperazin-1-yl)prop-1-yn-1-yl)pyridin-2-yl)-4-hydroxypiperidine-4-1,3-dione (33)

DMF (0.80 mL) and triethylamine (12 μL, 85 μmol, 5.0 eq.) were added sequentially to a dry 5 mL MWV equipped with a magnetic stir bar containing 4-(3-(3-fluoro-4-(4-(prop-2-yn-1-yl)piperazin-1-yl)phenyl)-2-methyl-3H-imidazo[4,5-b]pyridin-5-yl)pyridin-2-amine (A10) (7.5 mg, 17 μmol, 1.0 eq.), 2-(2,6-dioxopiperidin-3-yl)-4-(((1-(1-(4-hydroxy-1-(5-iodopyridin-2-yl)piperidine-4-carbonyl)piperidin-4-yl)-1H-pyrazol-4-yl)methyl)amino)isoindoline-1,3-dione (13 mg, 17 μmol, 1.0 eq.), PdCl₂ (dppf) (1.8 mg, 2.5 μmol, 0.15 eq.) and copper (I) iodide (1.0 mg, 5.3 μmol, 0.31 eq.). This was stirred for 24 hours at room temperature where it was concentrated under reduced pressure and purified by flash column chromatography to give 4-(((1-(1-(1-(5-(3-(4-(4-(5-(2-aminopyridin-4-yl)-2-methyl-3H-imidazo[4,5-b]pyridin-3-yl)-2-fluorophenyl)piperazin-1-yl)prop-1-yn-1-yl)pyridin-2-yl)-4-hydroxypiperidine-4-carbonyl)piperidin-4-yl)-1H-pyrazol-4-yl)methyl)amino)-2-(2,6-dioxopiperidin-3-yl)isoindoline-1,3-dione (33) (1.7 mg, 1.6 μmol, 9% yield), a yellow solid. LCMS (ESI) C₅₈H₅₈FN₁₅O₆ requires 1079.46, found 1080 (M/Z+H).

### Example 34. 3-(4-((1-(1-(1-(5-(3-(4-(4-(5-(2-aminopyridin-4-yl)-2-methyl-3H-imidazo[4,5-b]pyridin-3-yl)-2,6-difluorophenyl)piperazin-1-yl)prop-1-yn-1-yl)pyridin-2-yl)piperidine-4-carbonyl)piperidin-4-yl)-1H-pyrazol-4-yl)methoxy)phenyl)piperidine-2,6-dione (34)

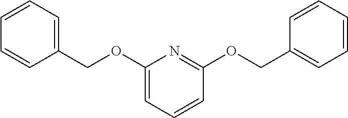 [SMILES: c1ccc(COc2cccc(OCc3ccccc3)n2)cc1]

### 2,6-bis(benzyloxy)pyridine (33a)

To a solution of potassium tert-butoxide (37.9 g, 337.8 mmol, 5.0 eq.) in THF (200 mL) was added benzyl alcohol (14.0 mL, 135.1 mmol, 2.0 eq.) at 0 C. 2,6-dichloropyridine (10.0 g, 67.5 mmol, 1.0 eq.) was added to the mixture at room temperature and stirred at 75° C. for 16 h. The reaction was quenched with saturated aqueous ammonium chloride at 0° C., diluted with ethyl acetate, and extracted with ethyl acetate. The combined organic layers were washed with brine, dried over sodium sulfate, filtered, and concentrated under reduced pressure to give a residue. The residue was triturated with pet ether to afford 2,6-bis(benzyloxy)pyridine (36a) (17.55 g, 60.24 mmol, 89% yield), a white solid. ¹H NMR (400 MHz, CDCl₃) δ 7.53 (t, J=7.9 Hz, 1H), 7.49-7.43 (m, 4H), 7.39 (tt, J=6.2, 1.1 Hz, 4H), 7.37-7.31 (m, 2H), 6.41 (d, J=7.9 Hz, 2H), 5.38 (s, 4H). ¹³C NMR (101 MHz, CDCl₃) δ 162.45, 141.25, 137.75, 128.58, 127.94, 127.88, 102.12, 67.76.

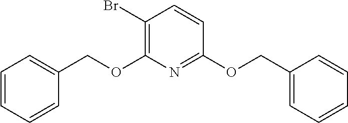 [SMILES: Brc1ccc(OCc2ccccc2)nc1OCc1ccccc1]

### 2,6-bis(benzyloxy)-3-bromopyridine (34b)

To a stirred solution of 2,6-bis(benzyloxy)pyridine (17.5 g, 60.2 mmol, 1.2 eq.) in acetonitrile (120 mL) was added NBS (8.57 g, 48.1 mmol, 1.0 eq.) portionwise and the reaction mixture was heated at 85° C. for 12 hours. The reaction mixture was cooled down to room temperature and partitioned between ethyl acetate and water. The organic layer was separated, dried over anhydrous sodium sulfate and concentrated under reduced pressure. The crude residue was purified by flash column chromatography using a 0-5% ethyl acetate in hexanes gradient to where the starting material co-eluted with the product. This white oily solid was triturated with hexanes and filtered to afford 2,6-bis(benzyloxy)-3-bromopyridine (34b) (14.0 g, 33 mmol, 68% yield, 87% purity), a white solid. ¹H NMR (500 MHz, CDCl₃) δ 7.59 (d, J=8.3 Hz, 1H), 7.37 (t, J=7.9 Hz, 2H), 7.34-7.26 (m, 6H), 7.26-7.21 (m, 2H), 6.24 (d, J=8.3 Hz, 1H), 5.35 (s, 2H), 5.23 (s, 2H). ¹³C NMR (126 MHz, CDCl₃) δ 161.34, 157.80, 144.07, 137.32, 137.26, 128.64, 128.05, 127.85, 127.39, 103.93, 96.08, 68.45, 68.26.

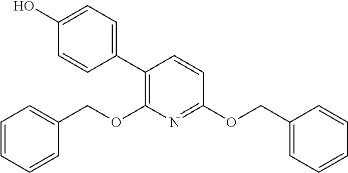 [SMILES: Oc1ccc(-c2ccc(OCc3ccccc3)nc2OCc2ccccc2)cc1]

### 4-(2,6-bis(benzyloxy)pyridin-3-yl) phenol (34c)

A 24 mL MWV equipped with a magnetic stir bar was loaded with 2,6-bis(benzyloxy)-3-bromopyridine (9.18 g, 24.81 mmol, 1.0 eq.), (4-hydroxyphenyl) boronic acid (3.57 g, 25.90 mmol, 1.04 eq.), cesium carbonate (14.07 g, 43.18 mmol, 1.74 eq.), and PdCl₂ (dppf) (1.58 g, 2.15 mmol, 0.08 eq.) was capped, purge with argon, then injected with degassed dioxane (101.8 mL) and D.I. water (25.4 mL) and was heated to 90° C. for 24 hours. The reaction mixture was cooled down, diluted with DCM, filtered through celite, and concentrated under reduced pressure. The crude residue was purified by flash column chromatography to give 4-(2,6-bis(benzyloxy)pyridin-3-yl) phenol (34c) (6.05 g, 15.78 mmol, 63% yield), as a white solid. ¹H NMR (500 MHz, CDCl₃) δ 7.43 (d, J=8.0 Hz, 1H), 7.35-7.28 (m, 4H), 7.26-7.09 (m, 9H), 6.72 (d, J=8.5 Hz, 2H), 6.33 (d, J=8.0 Hz, 1H), 5.29 (s, 2H), 5.23 (s, 2H). LCMS (ESI) C₂₅H₂₁NO₃ requires 383.45, found 384 (M/Z+H). LCMS (ESI) requires 383.15, found 384 (M/Z+H).

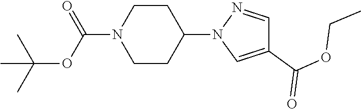 [SMILES: CCOC(=O)c1cnn(C2CCN(C(=O)OC(C)(C)C)CC2)c1]

### tert-butyl 4-(4-(ethoxycarbonyl)-1H-pyrazol-1-yl)piperidine-1-carboxylate (34d)

Ethyl 1H-pyrazole-4-carboxylate (3.77 g, 26.90 mmol, 1.0 eq.) and cesium carbonate (26.29 g, 80.69 mmol, 3.0 eq.) in DMF (75 mL) were stirred in a 250 mL RBF under an inert atmosphere for 30 minutes at room temperature. This was cooled down to 0° C. where tert-butyl 4-((methylsulfonyl)oxy)piperidine-1-carboxylate (7.51 g, 26.90 mmol, 1.0 eq.) was added and the reaction mixture was stirred at 90° C. for 24 hours. The reaction mixture was then poured into ice cold water where the resulting suspension was filtered, washed with water, and dried by vacuum. The off-white solid was used without further purification. tert-butyl 4-(4-(ethoxycarbonyl)-1H-pyrazol-1-yl)piperidine-1-carboxylate (34d) (6.50 g, 20.12 mmol, 74% yield). LCMS (ESI) C₁₆H₂₅N₃O₄ requires 323.18, found 268.37 (M/Z+H), carbamic acid, de-tBu in LCMS.

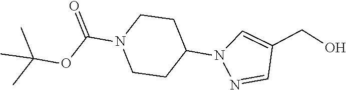 [SMILES: CC(C)(C)OC(=O)N1CCC(n2cc(CO)cn2)CC1]

### tert-butyl 4-(4-(hydroxymethyl)-1H-pyrazol-1-yl)piperidine-1-carboxylate (34e)

To a stirred solution of tert-butyl 4-(4-(ethoxycarbonyl)-1H-pyrazol-1-yl)piperidine-1-carboxylate (34d) (2.33 g, 7.21 mmol, 1.0 eq.) in THF (28.86 mL), diisobutylaluminum hydride (30.0 mL, 36.07 mmol, 1.2 molar, 5.0 eq.) was added dropwise at −78° C. and stirred at this same temperature for 4 hours under a nitrogen atmosphere. After complete consumption, as evidenced from TLC, the reaction was diluted with ethyl acetate and quenched with ammonium chloride. The organic phase was separated, dried over anhydrous sodium sulfate, and concentrated under reduced pressure to give tert-butyl 4-(4-(hydroxymethyl)-1H-pyrazol-1-yl)piperidine-1-carboxylate (34e) (1.83 g, 6.51 mmol, 90% yield) and was used without further purification. LCMS (ESI) C₁₄H₂₃N₃O₃ requires 281.17, found 282 (M/Z+H).

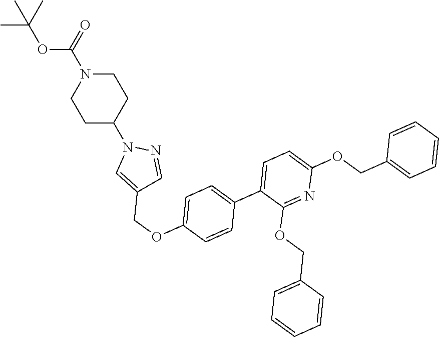 [SMILES: CC(C)(C)OC(=O)N1CCC(n2cc(COc3ccc(-c4ccc(OCc5ccccc5)nc4OCc4ccccc4)cc3)cn2)CC1]

### tert-butyl 4-(4-((4-(2,6-bis(benzyloxy)pyridin-3-yl) phenoxy)methyl)-1H-pyrazol-1-yl)piperidine-1-carboxylate (34f)

Tert-butyl 4-(4-(hydroxymethyl)-1H-pyrazol-1-yl)piperidine-1-carboxylate (34e) (734 mg, 2.61 mmol, 1.25 eq.), 4-(2,6-bis(benzyloxy)pyridin-3-yl) phenol (34c) (800 mg, 2.09 mmol, 1.0 eq.), and triphenylphosphine (822 mg, 3.13 mmol, 1.5 eq.) in THF (10.4 mL) was added DIAD (0.40 mL, 2.09 mmol, 1.0 eq.) dropwise at 0° C. and the reaction mixture was stirred at 60° C. for 12 hours. After 12 hours, there was still a lot of starting material so DEAD (0.58 mL, 3.34 mmol, 1.6 eq.) and triphenylphosphine (548 mg, 2.08 mmol, 1.0 eq.) were added. The reaction mixture was stirred at 70° C. for an additional 16 hours. The reaction mixture was then concentrated under reduced pressure, extracted with ethyl acetate and washed with brine. The organic layers were recombined, dried over anhydrous sodium sulfate and concentrated under reduced pressure. The crude residue was purified by flash column chromatography to give tert-butyl 4-(4-((4-(2,6-bis(benzyloxy)pyridin-3-yl) phenoxy)methyl)-1H-pyrazol-1-yl)piperidine-1-carboxylate (34f) (413 mg, 0.63 mmol, 30% yield). LCMS (ESI) C₃₉H₄₂N₄O₅ requires 646.31, found 647 (M/Z+H).

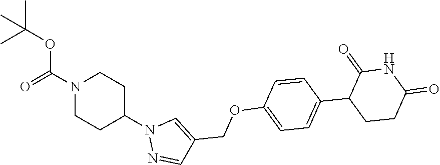 [SMILES: CC(C)(C)OC(=O)N1CCC(n2cc(COc3ccc(C4CCC(=O)NC4=O)cc3)cn2)CC1]

### tert-butyl 4-(4-((4-(2,6-dioxopiperidin-3-yl) phenoxy)methyl)-1H-pyrazol-1-yl)piperidine-1-carboxylate (34g)

Tert-butyl 4-(4-((4-(2,6-bis(benzyloxy)pyridin-3-yl) phenoxy)methyl)-1H-pyrazol-1-yl)piperidine-1-carboxylate (34f) (313 mg, 484 μmol, 1.0 eq.), palladium on carbon (155 mg, 1.46 mmol, 3.0 eq.), and palladium hydroxide on carbon (155 mg, 1.10 mmol, 2.28 eq.) under an atmosphere of nitrogen was added 1,4-dioxane (9.62 mL). The mixture was stirred at 25° C. for 24 hours under a hydrogen atmosphere. After completion of the reaction, the mixture was filtered through a pad of celite which did not get out all the palladium so a syringe filter was used. The filtrate was concentrated under reduced pressure to give tert-butyl 4-(4-((4-(2,6-dioxopiperidin-3-yl) phenoxy)methyl)-1H-pyrazol-1-yl)piperidine-1-carboxylate (34g) (200 mg, 0.42 mmol, 88% yield). LCMS (ESI) C₂₅H₃₂N₄O₅ requires 468.2, found 469.1 (M/Z+H).

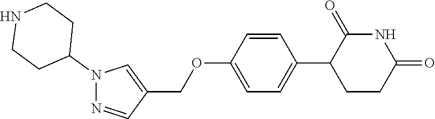 [SMILES: O=C1CCC(c2ccc(OCc3cnn(C4CCNCC4)c3)cc2)C(=O)N1]

### 3-(4-((1-(piperidin-4-yl)-1H-pyrazol-4-yl)methoxy)phenyl)piperidine-2,6-dione (34h)

Trifluoroacetic acid (0.75 mL, 9.82 mmol, 20.0 eq.) is added to a 20 mL dram vial containing tert-butyl 4-(4-((4-(2,6-dioxopiperidin-3-yl) phenoxy)methyl)-1H-pyrazol-1-yl)piperidine-1-carboxylate (34g) (230 mg, 0.49 mmol, 1.0 eq.) and DCM (3.00 mL). The reaction mixture was stirred for 3 hours where it is then concentrated under reduced pressure, extracted with DCM, and washed with a saturated solution of sodium bicarbonate. The organic layers were recombined, dried over sodium sulfate, and concentrated under reduced pressure to give 3-(4-((1-(piperidin-4-yl)-1H-pyrazol-4-yl)methoxy)phenyl)piperidine-2,6-dione (34h) (168 mg, 0.45 mmol, 92% yield). LCMS (ESI) C₂₀H₂₄N₄O₃ requires 368.18, found 369 (M/Z+H).

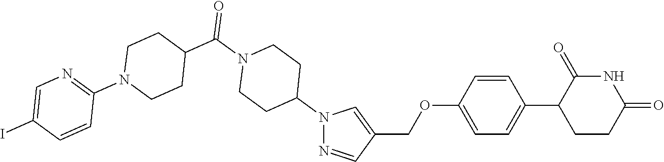 [SMILES: O=C1CCC(c2ccc(OCc3cnn(C4CCN(C(=O)C5CCN(c6ccc(I)cn6)CC5)CC4)c3)cc2)C(=O)N1]

### 3-(4-((1-(1-(1-(5-iodopyridin-2-yl)piperidine-4-carbonyl)piperidin-4-yl)-1H-pyrazol-4-yl)methoxy)phenyl)piperidine-2,6-dione (34i)

DMF (1.10 mL) and DIPEA (0.14 mL, 0.81 mmol, 6.0 eq.) were added sequentially to a reaction vessel containing HATU (62 mg, 0.16 mmol, 1.2 eq.), 3-(4-((1-(piperidin-4-yl)-1H-pyrazol-4-yl)methoxy)phenyl)piperidine-2,6-dione (34h) (50 mg, 0.14 mmol, 1.0 eq.), and 1-(5-iodopyridin-2-yl)piperidine-4-carboxylic acid (18b) (45 mg, 0.14 mmol, 1.0 eq.). The resulting solution was stirred at room temperature for 6 hours before the reaction mixture was diluted with water and extracted with ethyl acetate. The organic layer was washed with brine (4×), dried over sodium sulfate, and concentrated under reduced pressure. The crude residue was purified by flash column chromatography to give 3-(4-((1-(1-(1-(5-iodopyridin-2-yl)piperidine-4-carbonyl)piperidin-4-yl)-1H-pyrazol-4-yl)methoxy)phenyl)piperidine-2,6-dione (34i) (37 mg, 54 μmol, 40% yield), a white solid. ¹H NMR (500 MHz, CDCl₃) δ 8.30 (dd, J=2.4, 0.7 Hz., 1H), 7.99 (s, 1H), 7.64 (dd, J=8.9, 2.4 Hz, 1H), 7.58 (d, J=0.8 Hz, 1H), 7.50 (s, 1H), 7.17-7.11 (m, 2H), 6.98-6.92 (m, 2H), 6.51 (dd, J=9.0, 0.8 Hz, 1H), 4.95 (s, 2H), 4.75 (d, J=13.8 Hz, 1H), 4.40-4.32 (m, 1H), 4.28 (d, J=13.3 Hz, 2H), 4.09 (d, J=14.1 Hz, 1H), 3.79-3.66 (m, 1H), 3.25 (t, J=12.9 Hz, 1H), 2.95-2.86 (m, 2H), 2.83-2.70 (m, 3H), 2.70-2.62 (m, 1H), 2.34-2.19 (m, 2H), 2.03-1.74 (m, 6H), 1.47 (d, J=6.7 Hz, 1H), 1.43 (d, J=6.7 Hz, 1H). LCMS (ESI) C₃₁H₃₅IN₆₀₄ requires 682.17, found 682.9 (M/Z+H).

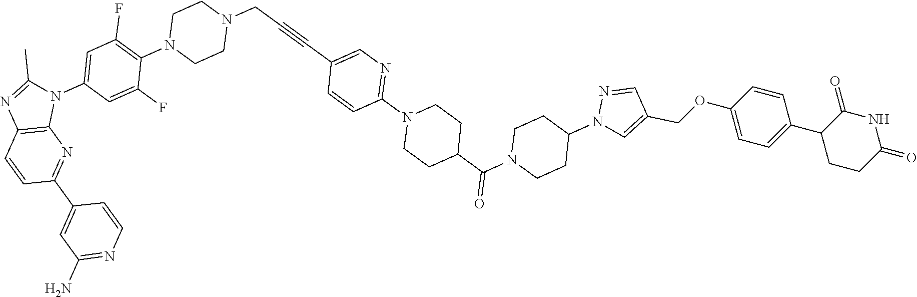 [SMILES: Cc1nc2ccc(-c3ccnc(N)c3)nc2n1-c1cc(F)c(N2CCN(CC#Cc3ccc(N4CCC(C(=O)N5CCC(n6cc(COc7ccc(C8CCC(=O)NC8=O)cc7)cn6)CC5)CC4)nc3)CC2)c(F)c1]

### 3-(4-((1-(1-(1-(5-(3-(4-(4-(5-(2-aminopyridin-4-yl)-2-methyl-3H-imidazo[4,5-b]pyridin-3-yl)-2,6-difluorophenyl)piperazin-1-yl)prop-1-yn-1-yl)pyridin-2-yl)piperidine-4-carbonyl)piperidin-4-yl)-1H-pyrazol-4-yl)methoxy)phenyl)piperidine-2,6-dione (34)

A flame dried 5 mL MWV equipped with a magnetic stir bar containing 3-(4-((1-(1-(1-(5-iodopyridin-2-yl)piperidine-4-carbonyl)piperidin-4-yl)-1H-pyrazol-4-yl)methoxy)phenyl)piperidine-2,6-dione (34i) (36 mg, 53 μmol, 1.0 eq.), 4-(3-(3,5-difluoro-4-(4-(prop-2-yn-1-yl)piperazin-1-yl)phenyl)-2-methyl-3H-imidazo[4,5-b]pyridin-5-yl)pyridin-2-amine (23e) (27 mg, 58 μmol, 1.1 eq.), copper (I) iodide (1.2 mg, 53 μmol, 0.12 eq.), and bis(triphenylphosphine) palladium (II) chloride (4.4 mg, 6.3 μmol, 0.12 eq.) was added DMF (0.80 mL) and triethylamine (22 μL, 0.16 mmol, 3.0 eq.) sequentially and was heated at room temperature for 16 hours where it was concentrated under reduced pressure and purified by flash column chromatography to give 3-(4-((1-(1-(1-(5-(3-(4-(4-(5-(2-aminopyridin-4-yl)-2-methyl-3H-imidazo[4,5-b]pyridin-3-yl)-2,6-difluorophenyl)piperazin-1-yl)prop-1-yn-1-yl)pyridin-2-yl)piperidine-4-carbonyl)piperidin-4-yl)-1H-pyrazol-4-yl)methoxy)phenyl)piperidine-2,6-dione (34) 8.0 mg, 8 μmol, 10% yield), a yellow solid. LCMS (ESI) C₅₆H₅₇F₂N₁₃₀₄ requires 1013.46, found 1014.2 (M/Z+H). HPLC: 89.7%.

### Example 35. Synthesis of 4-(((1-(1-(1-(2-(3-(4-(4-(5-(2-aminopyridin-4-yl)-2-methyl-3H-imidazo[4,5-b]pyridin-3-yl)-2,6-difluorophenyl)piperazin-1-yl)prop-1-yn-1-yl)pyrimidin-5-yl)piperidine-4-carbonyl)piperidin-4-yl)-1H-pyrazol-4-yl)methyl)amino)-2-(2,6-dioxopiperidin-3-yl)isoindoline-1,3-dione (35)

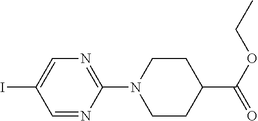 [SMILES: CCOC(=O)C1CCN(c2ncc(I)cn2)CC1]

### ethyl 1-(5-iodopyrimidin-2-yl)piperidine-4-carboxylate (35a)

To a solution of DMF (7.34 mL), DIPEA (2.17 mL, 12.4 mmol, 2.0 eq.), and ethyl piperidine-4-carboxylate (1.15 mL, 7.48 mmol, 1.2 eq.) was added 2-chloro-5-iodopyrimidine (1.50 g, 6.23 mmol, 1.0 eq.). The MWV was capped and stirred at 115° C. for 24 hours where it was cooled down to room temperature and extracted with ethyl acetate, washed with water, then brine. The combined extracts were dried over sodium sulfate, concentrated under reduced pressure, and purified by flash column chromatography. The product was further purified by washing and filtering with hexanes to give ethyl 1-(5-iodopyrimidin-2-yl)piperidine-4-carboxylate (35a) (2.17 g, 6.01 mmol, 96% yield), a white solid. LCMS (ESI) C₁₂H₁₆IN₃₀₂ requires 361.0, found 361.8 (M/Z+H).

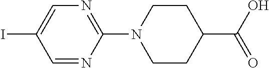 [SMILES: O=C(O)C1CCN(c2ncc(I)cn2)CC1]

### 1-(5-iodopyrimidin-2-yl)piperidine-4-carboxylic acid (35b)

To a solution of ethyl 1-(5-iodopyrimidin-2-yl)piperidine-4-carboxylate (35a) (2.10 g, 5.81 mmol, 1.0 eq.) in THF (30 mL) was added a solution of sodium hydroxide (410 mg, 5.12 mL, 10.2 mmol, 1.76 eq.), and the mixture was heated at 110° C. for 48 hours were it was cooled own and concentrated under reduced pressure. The residue was washed with 1M HCl, filtered, and washed with water and hexanes to give 1-(5-iodopyrimidin-2-yl)piperidine-4-carboxylic acid (35b) (1.57 g, 4.73 mmol, 81% yield), a white solid. LCMS (ESI) requires 332.9, found 333.8 (M/Z+H).

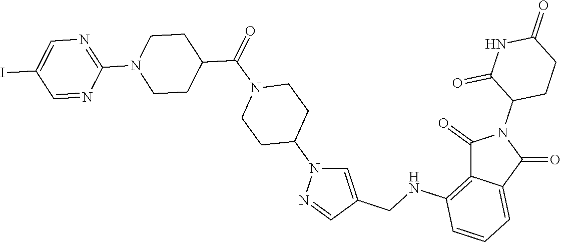 [SMILES: O=C1CCC(N2C(=O)c3cccc(NCc4cnn(C5CCN(C(=O)C6CCN(c7ncc(I)cn7)CC6)CC5)c4)c3C2=O)C(=O)N1]

### 2-(2,6-dioxopiperidin-3-yl)-4-(((1-(1-(1-(5-iodopyrimidin-2-yl)piperidine-4-carbonyl)piperidin-4-yl)-1H-pyrazol-4-yl)methyl)amino)isoindoline-1,3-dione (35c)

DMF (0.85 mL) and DIPEA (0.14 mL, 0.82 mmol, 6.0 eq.) were added sequentially to a reaction vessel containing HATU (63 mg, 0.16 mmol, 1.2 eq.), 2-(2,6-dioxopiperidin-3-yl)-4-(((1-(piperidin-4-yl)-1H-pyrazol-4-yl)methyl)amino)isoindoline-1,3-dione (60 mg, 0.14 mmol, 1.0 eq.), and 1-(5-iodopyrimidin-2-yl)piperidine-4-carboxylic acid (35b) (46 mg, 0.14 mmol, 1.0 eq.). The resulting solution was stirred at room temperature overnight where it was diluted with water and extracted with ethyl acetate. The organic layer was washed with brine (4×), dried over sodium sulfate, and concentrated under reduced pressure. The crude residue was purified by flash column chromatography to give 2-(2,6-dioxopiperidin-3-yl)-4-(((1-(1-(1-(5-iodopyrimidin-2-yl)piperidine-4-carbonyl)piperidin-4-yl)-1H-pyrazol-4-yl)methyl)amino)isoindoline-1,3-dione (35c) (68 mg, 91 μmol, 66% yield, eluting at 5% MeOH in DCM), a yellow solid. LCMS (ESI) C₃₂H₃₄IN₉O₅ requires 751.1, found 751.9 (M/Z+H).

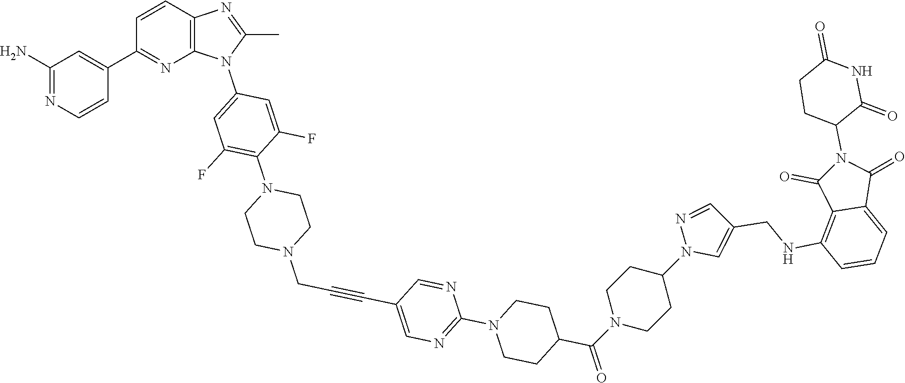 [SMILES: Cc1nc2ccc(-c3ccnc(N)c3)nc2n1-c1cc(F)c(N2CCN(CC#Cc3cnc(N4CCC(C(=O)N5CCC(n6cc(CNc7cccc8c7C(=O)N(C7CCC(=O)NC7=O)C8=O)cn6)CC5)CC4)nc3)CC2)c(F)c1]

### 4-(((1-(1-(1-(2-(3-(4-(4-(5-(2-aminopyridin-4-yl)-2-methyl-3H-imidazo[4,5-b]pyridin-3-yl)-2,6-difluorophenyl)piperazin-1-yl)prop-1-yn-1-yl)pyrimidin-5-yl)piperidine-4-carbonyl)piperidin-4-yl)-1H-pyrazol-4-yl)methyl)amino)-2-(2,6-dioxopiperidin-3-yl)isoindoline-1,3-dione (35)

DMF (0.70 mL) and triethylamine (14.6 μL, 104.5 μmol, 3.0 eq.) were added sequentially to a dry 5 mL MWV equipped with a magnetic stir bar under an inert atmosphere containing 4-(3-(3,5-difluoro-4-(4-(prop-2-yn-1-yl)piperazin-1-yl)phenyl)-2-methyl-3H-imidazo[4,5-b]pyridin-5-yl)pyridin-2-amine (16.0 mg, 34.8 μmol, 1.0 eq.), 3-(4-(((1-(1-(1-(2-iodopyrimidin-5-yl)piperidine-4-carbonyl)piperidin-4-yl)-1H-pyrazol-4-yl)methyl)amino)-1-oxoisoindolin-2-yl)piperidine-2,6-dione (25.6 mg, 34.8 μmol, 1.0 eq.), PdCl₂ (dppf) (2.0 mg, 2.7 μmol, 0.08 eq.) and copper (I) iodide (0.8 mg, 4.1 μmol, 0.12 eq.). This was stirred for 24 hours at room temperature where it was concentrated under reduced pressure and purified by flash column chromatography to give 4-(((1-(1-(1-(2-(3-(4-(4-(5-(2-aminopyridin-4-yl)-2-methyl-3H-imidazo[4,5-b]pyridin-3-yl)-2,6-difluorophenyl)piperazin-1-yl)prop-1-yn-1-yl)pyrimidin-5-yl)piperidine-4-carbonyl)piperidin-4-yl)-1H-pyrazol-4-yl)methyl)amino)-2-(2,6-dioxopiperidin-3-yl)isoindoline-1,3-dione (35) (19.6 mg, 18.1 μmol, 52% yield), a yellow solid. LCMS (ESI) C₅₇H₅₆F₂N₁₆O₅ requires 1082.4, found 1083.3 (M/Z+H).

### Example 36. Synthesis of 4-(((1-(1-(1-(5-(3-(4-(4-(4-(5-(2-aminopyridin-4-yl)-2-methyl-3H-imidazo[4,5-b]pyridin-3-yl)-2,6-difluorophenyl)piperazin-1-yl)piperidin-1-yl)prop-1-yn-1-yl)pyridin-2-yl)piperidine-4-carbonyl)piperidin-4-yl)-1H-pyrazol-4-yl)methyl)amino)-2-(2,6-dioxopiperidin-3-yl)isoindoline-1,3-dione (36)

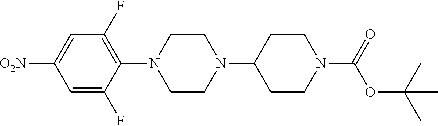 [SMILES: CC(C)(C)OC(=O)N1CCC(N2CCN(c3c(F)cc([N+](=O)[O-])cc3F)CC2)CC1]

### tert-butyl 4-(4-(2,6-difluoro-4-nitrophenyl)piperazin-1-yl)piperidine-1-carboxylate (36a)

To a solution of 1-(2,6-difluoro-4-nitrophenyl)piperazine (306 mg, 1.26 mmol, 1.0 eq.) and tert-butyl 4-oxopiperidine-1-carboxylate (501 mg, 2.52 mmol, 2.0 eq.) in 1,2-dichloroethane (8.39 mL) was added acetic acid (0.18 mL, 3.15 mmol, 2.5 eq.) and was stirred at room temperature for 30 minutes. Sodium triacetoxyborohydride (533 mg, 2.52 mmol, 2.0 eq.) was added and the reaction mixture was stirred for 16 hours where it was then quenched with a saturated solution of sodium bicarbonate and was extracted with ethyl acetate. The organic layer was washed with brine, dried over sodium sulfate, and concentrated under reduced pressure. The residue was purified by flash column chromatography using a hexanes/ethyl acetate gradient to give tert-butyl 4-(4-(2,6-difluoro-4-nitrophenyl)piperazin-1-yl)piperidine-1-carboxylate (36a) (620 mg, 1.1 mmol, 88% yield, 76% Purity), a red oil. LCMS (ESI) C₂₀H₂₈F₂N₄O₄ requires 426.2, found 427.6 (M/Z+H).

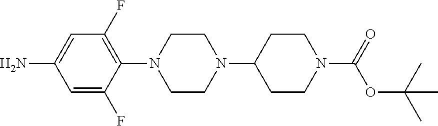 [SMILES: CC(C)(C)OC(=O)N1CCC(N2CCN(c3c(F)cc(N)cc3F)CC2)CC1]

### tert-butyl 4-(4-(4-amino-2,6-difluorophenyl)piperazin-1-yl)piperidine-1-carboxylate (36b)

To a 8 mL MWV equipped with a magnetic stir bar containing tert-butyl 4-(4-(2,6-difluoro-4-nitrophenyl)piperazin-1-yl)piperidine-1-carboxylate (36a) (620 mg, 1.10 mmol, 1.0 eq. 76% wt.) in ethanol (7.69 mL) and water (1.92 mL) was added ammonium chloride (35 mg, 0.66 mmol, 0.6 eq.) and was stirred at 40° C. iron (370 mg, 6.63 mmol, 6.0 eq.) was then added and heated to 90° C. and stirred for 4 hours. After cooling to room temperature, the reaction mixture was filtered and partitioned between DCM and saturated sodium bicarbonate. The aqueous phase was extracted with DCM (3×) and the combined organic phases were dried over sodium sulfate. The residue was redissolved in chloroform and concentrated under reduced pressure (3×) to give tert-butyl 4-(4-(4-amino-2,6-difluorophenyl)piperazin-1-yl)piperidine-1-carboxylate (36b) (305 mg, 0.38 mmol, 35% yield, 50% purity). LCMS (ESI) C₂₀H₃₀F₂N₄O₂ requires 396.2, found 397 (M/Z +H).

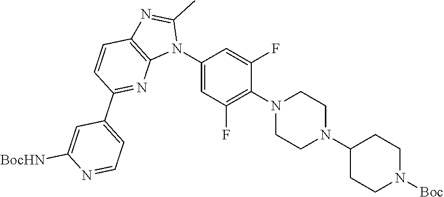 [SMILES: Cc1nc2ccc(-c3ccnc(NC(=O)OC(C)(C)C)c3)nc2n1-c1cc(F)c(N2CCN(C3CCN(C(=O)OC(C)(C)C)CC3)CC2)c(F)c1]

### tert-butyl 4-(4-(4-(5-(2-((tert-butoxycarbonyl)amino)pyridin-4-yl)-2-methyl-3H-imidazo[4,5-b]pyridin-3-yl)-2,6-difluorophenyl)piperazin-1-yl)piperidine-1-carboxylate (36c)

potassium phosphate (351 mg, 1.65 mmol, 6.0 eq.), tert-butyl (5-acetamido-6-chloro-[2,4′-bipyridin]-2′-yl)carbamate (A5) (200 mg, 0.27 mmol, 1.0 eq., 50% wt.), Pd₂dba₃ (10.1 mg, 11.0 μmol, 0.04 eq.), dicyclohexyl(2′,4′,6′-triisopropyl-[1,1′-biphenyl]-2-yl)phosphane (26.3 mg, 55.2 μmol, 0.20 eq.), and tert-butyl 4-(4-(4-amino-2,6-difluorophenyl)piperazin-1-yl)piperidine-1-carboxylate (36b) (219 mg, 0.55 mmol, 2.0 eq.) were added to a flame-dried 5 mL MWV equipped with a magnetic stir bar. The MWV was capped and purged with argon and degassed t-BuOH (0.68 mL) was added, and the reaction mixture was stirred at 90° C. for 16 hours. The reaction mixture was allowed to cool to room temperature and diluted with dichloromethane. The diluted mixture was filtered through Celite with the aid of dichloromethane and 5% MeOH in DCM. The filtrate was concentrated under reduced pressure and purified by flash column chromatography to give tert-butyl 4-(4-(4-(5-(2-((tert-butoxycarbonyl)amino)pyridin-4-yl)-2-methyl-3H-imidazo[4,5-b]pyridin-3-yl)-2,6-difluorophenyl)piperazin-1-yl)piperidine-1-carboxylate (36c) (130 mg, 0.18 mmol, 66% yield), a light-brown solid. LCMS (ESI) C₃₇H₄₆F₂N₈₀₄ requires 704.3, found 705.1 (M/Z+H).

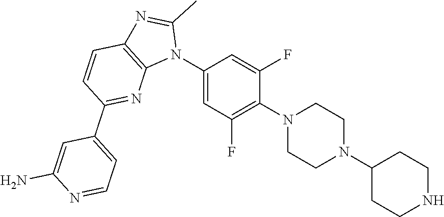 [SMILES: Cc1nc2ccc(-c3ccnc(N)c3)nc2n1-c1cc(F)c(N2CCN(C3CCNCC3)CC2)c(F)c1]

### 4-(3-(3,5-difluoro-4-(4-(piperidin-4-yl)piperazin-1-yl)phenyl)-2-methyl-3H-imidazo[4,5-b]pyridin-5-yl)pyridin-2-amine (36d)

HCl (346 mg, 2.37 mL, 4 molar, 35 Eq, 9.48 mmol) was added to a 20 mL MWV equipped with a magnetic stir bar containing tert-butyl 4-(4-(4-(5-(2-((tert-butoxycarbonyl)amino)pyridin-4-yl)-2-methyl-3H-imidazo[4,5-b]pyridin-3-yl)-2,6-difluorophenyl)piperazin-1-yl)piperidine-1-carboxylate (36c) (191 mg, 1 Eq, 271 μmol). A 4M solution of HCl in 1,4-dioxane (2.37 mL, 9.48 mmol, 35 eq.) was added to a 5 mL MWV equipped with a magnetic stir bar containing tert-butyl 4-(4-(4-(5-(2-((tert-butoxycarbonyl)amino)pyridin-4-yl)-2-methyl-3H-imidazo[4,5-b]pyridin-3-yl)-2,6-difluorophenyl)piperazin-1-yl)piperidine-1-carboxylate (191 mg, 0.27 mmol, 1.0 eq.). The reaction mixture was stirred overnight and concentrated under reduced pressure. LCMS showed that the mono-boc intermediate was still present so dichloromethane (2.00 mL) and trifluoroacetic acid (0.49 mL, 6.35 mmol, 25 eq.) were added. The reaction mixture was stirred for 4 hours where it was then concentrated under reduced pressure and extracted with DCM and washed with a saturated solution of 1M NaOH. The organic layer was dried over magnesium sulfate, filtered, and concentrated under reduced pressure to give 4-(3-(3,5-difluoro-4-(4-(piperidin-4-yl)piperazin-1-yl)phenyl)-2-methyl-3H-imidazo[4,5-b]pyridin-5-yl)pyridin-2-amine (36d) (84 mg, 0.16 mmol, 65%), a brown solid and was used in the next step without further purification.

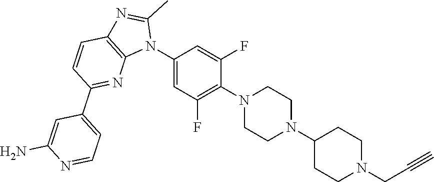 [SMILES: C#CCN1CCC(N2CCN(c3c(F)cc(-n4c(C)nc5ccc(-c6ccnc(N)c6)nc54)cc3F)CC2)CC1]

### 4-(3-(3,5-difluoro-4-(4-(1-(prop-2-yn-1-yl)piperidin-4-yl)piperazin-1-yl)phenyl)-2-methyl-3H-imidazo[4,5-b]pyridin-5-yl)pyridin-2-amine (36e)

Propargyl bromide (16 μL, 0.15 mmol, 0.89 eq.) was added to a 5 mL MWV equipped with a magnetic stir bar containing 4-(3-(3,5-difluoro-4-(4-(piperidin-4-yl)piperazin-1-yl)phenyl)-2-methyl-3H-imidazo[4,5-b]pyridin-5-yl)pyridin-2-amine (35e) (84.5 mg, 0.16 mmol, 1.0 eq.) and potassium carbonate (74 mg, 0.53 mmol, 3.2 eq.) in DMF (1.5 mL). The reaction mixture was stirred at room temperature for 16 hours where it was then diluted with a saturated solution of sodium bicarbonate (5 mL). The mixture was extracted with DCM (3×15 mL) and the organic layers were recombined, dried over sodium sulfate, and concentrated under reduced pressure. The crude residue was purified by flash column chromatography using a DCM/MeOH gradient to afford 4-(3-(3,5-difluoro-4-(4-(1-(prop-2-yn-1-yl)piperidin-4-yl)piperazin-1-yl)phenyl)-2-methyl-3H-imidazo[4,5-b]pyridin-5-yl)pyridin-2-amine (36e) (20.0 mg, 36.9 μmol, 22% yield). LCMS (ESI) C₃₀H₃₂F₂N₈ requires 542.2, found 543 (M/Z+H).

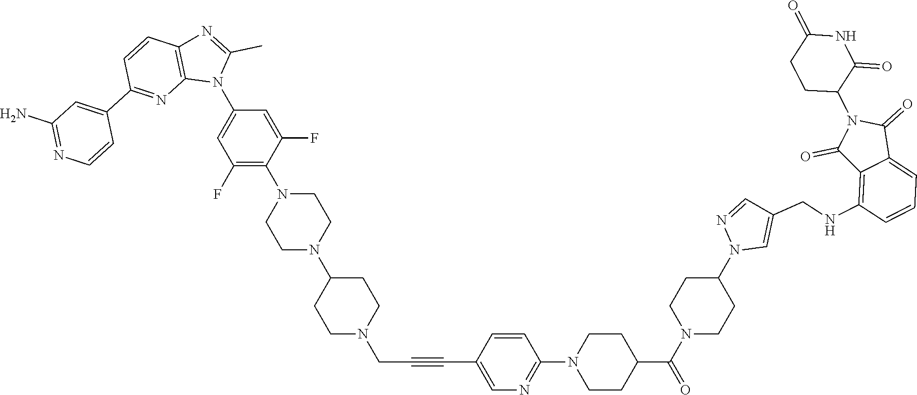 [SMILES: Cc1nc2ccc(-c3ccnc(N)c3)nc2n1-c1cc(F)c(N2CCN(C3CCN(CC#Cc4ccc(N5CCC(C(=O)N6CCC(n7cc(CNc8cccc9c8C(=O)N(C8CCC(=O)NC8=O)C9=O)cn7)CC6)CC5)nc4)CC3)CC2)c(F)c1]

### 4-(((1-(1-(1-(5-(3-(4-(4-(4-(5-(2-aminopyridin-4-yl)-2-methyl-3H-imidazo[4,5-b]pyridin-3-yl)-2,6-difluorophenyl)piperazin-1-yl)piperidin-1-yl)prop-1-yn-1-yl)pyridin-2-yl)piperidine-4-carbonyl)piperidin-4-yl)-1H-pyrazol-4-yl)methyl)amino)-2-(2,6-dioxopiperidin-3-yl)isoindoline-1,3-dione (36)

DMF (1.42 mL) and triethylamine (25.7 μL, 184 μmol, 5.0 eq.) were added sequentially to a dry 5 mL MWV equipped with a magnetic stir bar containing 4-(3-(3,5-difluoro-4-(4-(1-(prop-2-yn-1-yl)piperidin-4-yl)piperazin-1-yl)phenyl)-2-methyl-3H-imidazo[4,5-b]pyridin-5-yl)pyridin-2-amine (36f) (20.0 mg, 36.9 μmol, 1.0 eq.), 2-(2,6-dioxopiperidin-3-yl)-4-(((1-(1-(1-(5-iodopyridin-2-yl)piperidine-4-carbonyl)piperidin-4-yl)-1H-pyrazol-4-yl)methyl)amino)isoindoline-1,3-dione (D7) (27.7 mg, 36.9 μmol, 1.0 eq.), PdCl₂ (dppf) (3.5 mg, 4.7 μmol, 0.13 eq.) and copper (I) iodide (1.75 mg, 9.2 μmol, 0.25 eq.). This was stirred for 24 hours at room temperature where it was concentrated under reduced pressure and purified by flash column chromatography. 4-(((1-(1-(1-(5-(3-(4-(4-(4-(5-(2-aminopyridin-4-yl)-2-methyl-3H-imidazo[4,5-b]pyridin-3-yl)-2,6-difluorophenyl)piperazin-1-yl)piperidin-1-yl)prop-1-yn-1-yl)pyridin-2-yl)piperidine-4-carbonyl)piperidin-4-yl)-1H-pyrazol-4-yl)methyl)amino)-2-(2,6-dioxopiperidin-3-yl)isoindoline-1,3-dione (36) (14.1 mg, 12.1 μmol, 32% yield), a yellow solid. LCMS (ESI) C₆₃H₆₆F₂N₁₆O₅ requires 1164.5, found 583 (M/2+H).

### Example 37. Synthesis of 4-(((1-(1-(1-(5-(3-((2S,6R)-4-(4-(5-(2-aminopyridin-4-yl)-2-methyl-3H-imidazo[4,5-b]pyridin-3-yl)-2,6-difluorophenyl)-2,6-dimethylpiperazin-1-yl)prop-1-yn-1-yl)pyridin-2-yl)piperidine-4-carbonyl)piperidin-4-yl)-1H-pyrazol-4-yl)methyl)amino)-2-(2,6-dioxopiperidin-3-yl)isoindoline-1,3-dione (37)

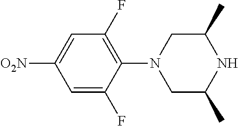 [SMILES: C[C@@H]1CN(c2c(F)cc([N+](=O)[O-])cc2F)C[C@H](C)N1]

### (3S,5R)-1-(2,6-difluoro-4-nitrophenyl)-3,5-dimethylpiperazine (37a)

To a solution of 1,2,3-trifluoro-5-nitrobenzene (543 mg, 3.07 mmol, 1.0 eq.) in DMSO (1.53 mL) was added (2S,6R)-2,6-dimethylpiperazine (350 mg, 3.07 mmol, 1.0 eq.) portion wise and the resulting mixture was stirred at room temperature until the solution became homogeneous. The reaction mixture was heated to 90° C. and stirred for 16 hours where it was allowed to cool to room temperature and concentrated under reduced pressure. The crude residue was extracted with ethyl acetate, washed with water, the organic layer was dried over sodium sulfate and concentrated under reduced pressure and used in the next step without further purification. LCMS (ESI) C₁₂H₁₅F₂N₃₀₂ requires 271.1, found 271.9 (M/Z+H).

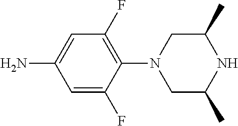 [SMILES: C[C@@H]1CN(c2c(F)cc(N)cc2F)C[C@H](C)N1]

### 4-(3-(3,5-difluoro-4-(4-(1-(prop-2-yn-1-yl)azetidin-3-yl)piperazin-1-yl)phenyl)-2-methyl-3H-imidazo[4,5-b]pyridin-5-yl)pyridin-2-amine (37b)

To a 8 mL MWV equipped with a magnetic stir bar containing (3S,5R)-1-(2,6-difluoro-4-nitrophenyl)-3,5-dimethylpiperazine (37a) (550 mg, 2.03 mmol, 1.0 eq.) in ethanol (14.1 mL) and water (3.53 mL) was added ammonium chloride (47 mg, 0.88 mmol, 0.4 eq.) and was stirred at 40° C. Iron (496 mg, 8.88 mmol, 4.4 eq.) was then added and heated to 90° C. and stirred for 4 hours. After cooling to room temperature, the reaction mixture was filtered and partitioned between DCM and a saturated solution of sodium bicarbonate. The aqueous phase was extracted with

DCM (3×) and the combined organic phases were dried over sodium sulfate, filtered, and concentrated under reduced pressure. The crude residue was purified by flash column chromatography using a DCM/MeOH gradient to give 4-((3S,5R)-3,5-dimethylpiperazin-1-yl)-3,5-difluoroaniline (37b) (214 mg, 0.88 mmol, 43% yield), an orange solid. LCMS (ESI)

C₁₂H₁₇F₂N₃ requires 241.1, found 242.0. (M/Z+H).

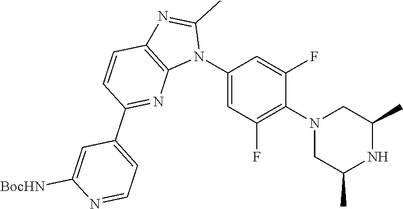 [SMILES: Cc1nc2ccc(-c3ccnc(NC(=O)OC(C)(C)C)c3)nc2n1-c1cc(F)c(N2C[C@@H](C)N[C@@H](C)C2)c(F)c1]

tert-butyl (4-(3-(4-((3S,5R)-3,5-dimethylpiperazin-1-yl)-3,5-difluorophenyl)-2-methyl-3H-imidazo[4,5-b]pyridin-5-yl)pyridin-2-yl)carbamate (37c)

Potassium phosphate (398 mg, 1.88 mmol, 3.0 eq.), tert-butyl (5-acetamido-6-chloro-[2,4′-bipyridin]-2′-yl)carbamate (A5) (227 mg, 0.62 mmol, 1.0 eq.), Pd₂dba₃ (11 mg, 12.5 μmol, 0.02 eq.), dicyclohexyl(2′,4′,6′-triisopropyl-[1,1′-biphenyl]-2-yl)phosphane (29 mg, 62.6 μmol, 0.10 eq.), and 4-((3S,5R)-3,5-dimethylpiperazin-1-yl)-3,5-difluoroaniline (36b) (214 mg, 0.88 mmol, 1.42 eq.) were added to a flame-dried 5 mL MWV equipped with a magnetic stir bar. The MWV was capped and purged with argon where degassed t-BuOH (1.56 mL) was added, and the reaction mixture was stirred at 90° C. for 24 hours. The reaction mixture was allowed to cool to room temperature and was diluted with dichloromethane. The diluted mixture was filtered through Celite with the aid of dichloromethane and 5% MeOH in DCM. The filtrate was concentrated under reduced pressure and purified by flash column chromatography to give tert-butyl (4-(3-(4-((3S,5R)-3,5-dimethylpiperazin-1-yl)-3,5-difluorophenyl)-2-methyl-3H-imidazo[4,5-b]pyridin-5-yl)pyridin-2-yl)carbamate (37c) (185 mg, 0.23 mmol, 37% yield, 69% purity), a light-brown solid. LCMS (ESI) C₂₉H₃₃F₂N₇₀₂ requires 549.2, found 550.1 (M/Z+H).

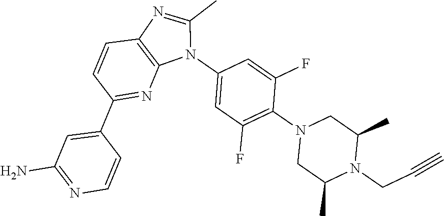 [SMILES: C#CCN1[C@H](C)CN(c2c(F)cc(-n3c(C)nc4ccc(-c5ccnc(N)c5)nc43)cc2F)C[C@@H]1C]

### 4-(3-(4-((3R,5S)-3,5-dimethyl-4-(prop-2-yn-1-yl)piperazin-1-yl)-3,5-difluorophenyl)-2-methyl-3H-imidazo[4,5-b]pyridin-5-yl)pyridin-2-amine (37d)

Propargyl bromide (19.5 μL, 0.18 mmol, 1.0 eq., 80% wt.) was added to a 5 mL MWV equipped with a magnetic stir bar containing tert-butyl (4-(3-(4-((3R,5S)-3,5-dimethylpiperazin-1-yl)-3,5-difluorophenyl)-2-methyl-3H-imidazo[4,5-b]pyridin-5-yl)pyridin-2-yl)carbamate (37c) (100 mg, 0.18 mmol, 1.0 eq.), potassium carbonate (75.4 mg, 0.54 mmol, 1.0 eq.), and potassium iodide (30 mg, 0.18 mmol, 1.0 eq.) in DMF (1.82 mL). The reaction mixture was heated at 90° C. for 2 hours where it was cooled down to room temperature and washed with saturated sodium bicarbonate (5 mL). The mixture was extracted with DCM (3×15 mL) and the organic layers were recombined, dried over sodium sulfate, and concentrated under reduced pressure. The crude residue was purified by flash column chromatography using a DCM/MeOH gradient to afford 4-(3-(4-((3R,5S)-3,5-dimethyl-4-(prop-2-yn-1-yl)piperazin-1-yl)-3,5-difluorophenyl)-2-methyl-3H-imidazo[4,5-b]pyridin-5-yl)pyridin-2-amine (37d) (21 mg, 43 μmol, 24% yield), a brown semisolid. LCMS (ESI) C₂₇H₂₇F₂N₇ requires 487.2, found 488.0 (M/Z+H). Note: Originally attempted with 4-bromo-1-butyne and heated to 125 C with no reaction and gave de-boc of 2-aminopyridine. To this mixture propargyl bromide was added.

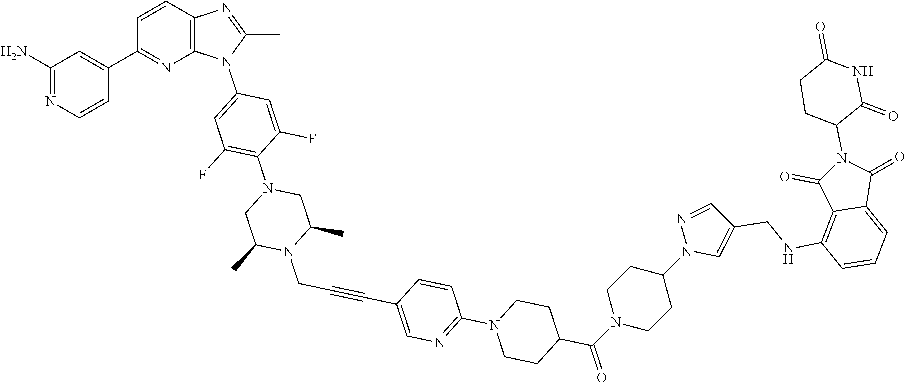 [SMILES: Cc1nc2ccc(-c3ccnc(N)c3)nc2n1-c1cc(F)c(N2C[C@@H](C)N(CC#Cc3ccc(N4CCC(C(=O)N5CCC(n6cc(CNc7cccc8c7C(=O)N(C7CCC(=O)NC7=O)C8=O)cn6)CC5)CC4)nc3)[C@@H](C)C2)c(F)c1]

### 4-(((1-(1-(1-(5-(3-((2S,6R)-4-(4-(5-(2-aminopyridin-4-yl)-2-methyl-3H-imidazo[4,5-b]pyridin-3-yl)-2,6-difluorophenyl)-2,6-dimethylpiperazin-1-yl)prop-1-yn-1-yl)pyridin-2-yl)piperidine-4-carbonyl)piperidin-4-yl)-1H-pyrazol-4-yl)methyl)amino)-2-(2,6-dioxopiperidin-3-yl)isoindoline-1,3-dione (37)

DMF (1.70 mL) and triethylamine (22.4 mg, 30.9 μL, 0.22 mmol, 5.0 eq.) were added sequentially to a dry 5 mL MWV equipped with a magnetic stir bar containing 4-(3-(4-((3S,5R)-3,5-dimethyl-4-(prop-2-yn-1-yl)piperazin-1-yl)-3,5-difluorophenyl)-2-methyl-3H-imidazo[4,5-b]pyridin-5-yl)pyridin-2-amine (37d) (21.6 mg, 44.3 μmol, 1.0 eq.), 2-(2,6-dioxopiperidin-3-yl)-4-(((1-(1-(1-(5-iodopyridin-2-yl)piperidine-4-carbonyl)piperidin-4-yl)-1H-pyrazol-4-yl)methyl)amino)isoindoline-1,3-dione (D7) (33.3 mg, 44.3 μmol, 1.0 eq.), PdCl₂ (dppf) (4.2 mg, 5.76 μmol, 0.13 eq.) and copper (I) iodide (2.1 mg, 11.1 μmol, 0.25 eq.). This reaction mixture was stirred under an inert atmosphere at room temperature for 24 hours where it was concentrated under reduced pressure and purified by flash column chromatography using a DCM/MeOH gradient to give 4-(((1-(1-(1-(5-(3-((2S,6R)-4-(4-(5-(2-aminopyridin-4-yl)-2-methyl-3H-imidazo[4,5-b]pyridin-3-yl)-2,6-difluorophenyl)-2,6-dimethylpiperazin-1-yl)prop-1-yn-1-yl)pyridin-2-yl)piperidine-4-carbonyl)piperidin-4-yl)-1H-pyrazol-4-yl)methyl)amino)-2-(2,6-dioxopiperidin-3-yl)isoindoline-1,3-dione (37) (14.3 mg, 12.9 μmol, 29% yield), a yellow solid. ¹H NMR (500 MHz, CDCl₃) δ 8.27 (s, 1H), 8.14 (s, 1H), 8.05 (d, J=8.3 Hz, 1H), 7.71 (d, J=8.3 Hz, 1H), 7.50 (d, J=8.0 Hz, 3H), 7.40 (s, 1H), 7.19 (s, 1H), 7.13 (d, J=6.7 Hz, 2H), 6.99 (d, J=8.7 Hz, 2H), 6.92 (d, J=8.6 Hz, 1H), 6.58 (d, J=8.9 Hz, 1H), 6.41 (s, 1H), 4.93-4.87 (m, 1H), 4.83 (s, 2H), 4.74 (s, 1H), 4.49-4.25 (m, 5H), 4.07 (s, 1H), 3.90 (s, 2H), 3.38-3.18 (m, 3H), 3.14 (t, J=11.0 Hz, 1H), 3.01 (s, 1H), 2.98-2.85 (m, 1H), 2.77 (m, 3H), 2.60 (s, 3H), 2.25 (s, 1H), 2.13 (s, 3H), 1.99-1.72 (m, 11H), 1.18 (s, 3H), 1.17 (s, 3H). ¹³C NMR (126 MHz, CDCl₃) δ 172.96, 171.43, 169.50, 168.84, 167.53, 161.19, 159.10, 158.50, 157.78, 156.52, 154.10, 151.39, 149.47, 148.77, 148.56, 148.07, 146.37, 140.28, 138.18, 136.21, 134.91, 132.51, 127.17, 125.56, 118.07, 116.83, 116.74, 112.04, 111.82, 110.52, 108.27, 106.17, 83.37, 82.87, 60.14, 59.12, 58.07, 53.73, 48.98, 45.62, 44.74, 44.30, 40.84, 40.80, 38.62, 37.72, 37.66, 33.41, 33.36, 32.06, 31.51, 29.71, 28.22, 27.97, 22.84, 22.70, 17.29, 17.28, 15.34, 1.03. 19F NMR (470 MHz, CDCl₃) δ -118. LCMS (ESI) C₆₀H₆₁F₂N₁₅O₅ requires 1109.4, found 1110.4 (M/Z+H).

### Example 38. Synthesis of 4-(((1-(1-(1-(5-(4-(4-(4-(5-(2-aminopyridin-4-yl)-2-methyl-3H-imidazo[4,5-b]pyridin-3-yl)-2-methoxyphenyl)piperazin-1-yl) but-1-yn-1-yl)pyridin-2-yl)piperidine-4-carbonyl)piperidin-4-yl)-1H-pyrazol-4-yl)methyl)amino)-2-(2,6-dioxopiperidin-3-yl)isoindoline-1,3-dione (38)

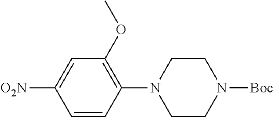 [SMILES: COc1cc([N+](=O)[O-])ccc1N1CCN(C(=O)OC(C)(C)C)CC1]

### tert-butyl 4-(2-methoxy-4-nitrophenyl)piperazine-1-carboxylate (38a)

To an 8 mL MWV equipped with a magnetic stir bar containing tert-butyl piperazine-1-carboxylate (1.08 g, 5.84 mmol, 1.0 eq.), potassium carbonate (2.42 g, 17.53 mmol, 3.0 eq.), in DMF (5.84 mL) was added 1-fluoro-2-methoxy-4-nitrobenzene (1.00 g, 5.84 mmol, 1.0 eq.). The reaction mixture was stirred at 90° C. for 18 hours where the resulting mixture was cooled to room temperature and extracted with ethyl acetate (2×20 mL) and washed with water. The organic layer was then washed with water and brine (5×10 mL), dried over sodium sulfate, filtered, and concentrated under reduced pressure. The crude residue was purified by flash column chromatography to give tert-butyl 4-(2-methoxy-4-nitrophenyl)piperazine-1-carboxylate (38a) (1.61 g, 4.77 mmol, 81% yield), an orange solid. LCMS (ESI) C₁₆H₂₃N₃O₅ requires 337.1, found 338.6 (M/Z+H).

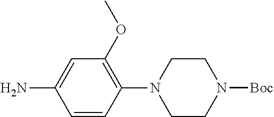 [SMILES: COc1cc(N)ccc1N1CCN(C(=O)OC(C)(C)C)CC1]

### tert-butyl 4-(4-amino-2-methoxyphenyl)piperazine-1-carboxylate (38b)

To a 8 mL MWV equipped with a magnetic stir bar containing tert-butyl 4-(2-methoxy-4-nitrophenyl)piperazine-1-carboxylate (38a) (1.61 g,, 4.77 mmol, 1.0 eq.) in ethanol (33.2 mL) and water (8.3 mL) was added ammonium chloride (107 mg, 2.00 mmol, 0.41 eq.) and was stirred at 40° C. Iron (1.10 g, 20.05 mmol, 4.20 eq.) was then added and heated to 90° C. and stirred for 2 hours. After cooling to room temperature, the reaction mixture was filtered and partitioned between DCM and a saturated solution of sodium bicarbonate. The aqueous phase was extracted with DCM (3×) and the combined organic phases were dried over sodium sulfate and was concentrated under reduced pressure. The crude residue was purified by flash column chromatography using a DCM/MeOH gradient to give tert-butyl 4-(4-amino-2-methoxyphenyl)piperazine-1-carboxylate (38b) (1.04 g, 3.40 mmol, 71% yield), a light brown-solid. LCMS (ESI) C₁₆H₂₅N₃O₃ requires 307.1, found 308.

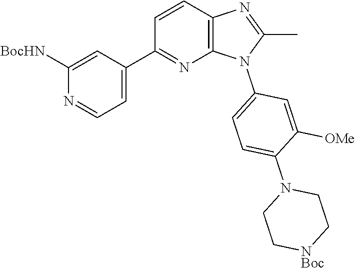 [SMILES: COc1cc(-n2c(C)nc3ccc(-c4ccnc(NC(=O)OC(C)(C)C)c4)nc32)ccc1N1CCN(C(=O)OC(C)(C)C)CC1]

### tert-butyl 4-(4-(5-(2-((tert-butoxycarbonyl)amino)pyridin-4-yl)-2-methyl-3H-imidazo[4,5-b]pyridin-3-yl)-2-methoxyphenyl)piperazine-1-carboxylate (38c)

Potassium phosphate (351 mg, 1.65 mmol, 3.0 eq.), tert-butyl (5-acetamido-6-chloro-[2,4′-bipyridin]-2′-yl)carbamate (A5) (200 mg, 0.55 mmol, 1.0 eq.), tert-butyl 4-(4-amino-2-methoxyphenyl)piperazine-1-carboxylate (38b) (169 mg, 0.55 mmol, 1.0 eq.), dicyclohexyl(2′,4′,6′-triisopropyl-[1,1′-biphenyl]]-2-yl)phosphane (26.3 mg, 55.1 μmol, 0.10 eq.), and Pd₂(dba)₃ (10.1 mg, 11.0 μmol, 0.02 eq.) were added to a flame-dried 5 mL MWV equipped with a magnetic stir bar. The MWV was capped and purged with argon where degassed t-BuOH (1.38 mL, 0.40M) was added and the reaction mixture was stirred at 90° C. for 24 hours. The reaction mixture was allowed to cool to room temperature and diluted with dichloromethane. The diluted mixture was filtered through Celite with the aid of dichloromethane and 5% MeOH in DCM. The filtrate was concentrated under reduced pressure and purified by flash column chromatography to give tert-butyl 4-(4-(5-(2-((tert-butoxycarbonyl)amino)pyridin-4-yl)-2-methyl-3H-imidazo[4,5-b]pyridin-3-yl)-2-methoxyphenyl)piperazine-1-carboxylate (38c) (244 mg, 0.39 mmol, 71% yield), a brown solid.

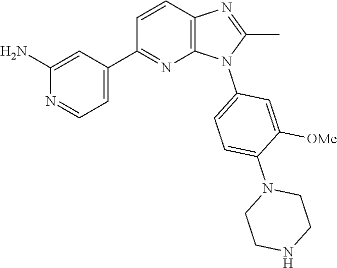 [SMILES: COc1cc(-n2c(C)nc3ccc(-c4ccnc(N)c4)nc32)ccc1N1CCNCC1]

### 4-(3-(3-methoxy-4-(piperazin-1-yl)phenyl)-2-methyl-3H-imidazo[4,5-b]pyridin-5-yl)pyridin-2-amine, Trifluoroacetic acid, Trifluoroacetic acid (38d)

4M HCl in 1,4-dioxane (3.24 mL, 13.0 mmol, 35.0 eq.) was added to a 20 mL scintillation equipped with a magnetic stir bar containing tert-butyl 4-(4-(5-(2-((tert-butoxycarbonyl)amino)pyridin-4-yl)-2-methyl-3H-imidazo[4,5-b]pyridin-3-yl)-2-methoxyphenyl)piperazine-1-carboxylate (38c) (228 mg, 0.37 mmol, 1.0 eq.) and the reaction mixture was stirred for 24 hours at room temperature where it was then concentrated under reduced pressure and extracted with DCM. The organic layer was washed with a saturated solution of sodium bicarbonate then dried over magnesium sulfate, filtered, and concentrated under reduced pressure to give the mono-boc (Boc-2-aminopyridine). Trifluoroacetic acid (0.73 mL, 9.48 mmol, 25.0 eq.) and DCM (2.19 mL) was added, and the reaction mixture was stirred for 24 hours where it was then concentrated under reduced pressure, extracted with DCM and washed with a saturated solution of sodium bicarbonate. The organic layer was dried over magnesium sulfate, filtered, and concentrated under reduced pressure to give 4-(3-(3-methoxy-4-(piperazin-1-yl)phenyl)-2-methyl-3H-imidazo[4,5-b]pyridin-5-yl)pyridin-2-amine (38d), a black solid, and was used without further purification.

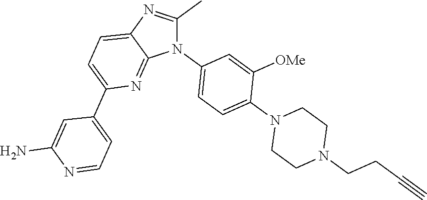 [SMILES: C#CCCN1CCN(c2ccc(-n3c(C)nc4ccc(-c5ccnc(N)c5)nc43)cc2OC)CC1]

### 4-(3-(4-(4-(but-3-yn-1-yl)piperazin-1-yl)-3-methoxyphenyl)-2-methyl-3H-imidazo[4,5-b]pyridin-5-yl)pyridin-2-amine (38e)

4-Bromobut-1-yne (15.3 μL, 0.16 mmol, 1.05 eq.) was added to a 5 mL MWV equipped with a magnetic stir bar containing 4-(3-(3-methoxy-4-(piperazin-1-yl)phenyl)-2-methyl-3H-imidazo[4,5-b]pyridin-5-yl)pyridin-2-amine, *2TFA (38d) (100 mg, 0.15 mmol, 1.0 eq.) and potassium carbonate (68 mg, 0.49 mmol, 3.2 eq.) in DMF (1.5 mL) The reaction mixture was stirred at 0° C. and after 1.5 hours the reaction proceeded slowly (LCMS). The reaction mixture was then heated to 80° C. for 4 hours then cooled down to room temperature, washed with a saturated solution of sodium bicarbonate (5 mL) and extracted with DCM (3×15 mL). The organic layers were recombined, dried over sodium sulfate, concentrated under reduced pressure, and purified by flash column chromatography using a DCM/MeOH gradient to give 4-(3-(4-(4-(but-3-yn-1-yl)piperazin-1-yl)-3-methoxyphenyl)-2-methyl-3H-imidazo[4,5-b]pyridin-5-yl)pyridin-2-amine (38e) (20.7 mg, 44.3 μmol, 28% yield). LCMS (ESI) C₂₇H₂₉N₇O requires 476.2, found 477 (M/Z+H).

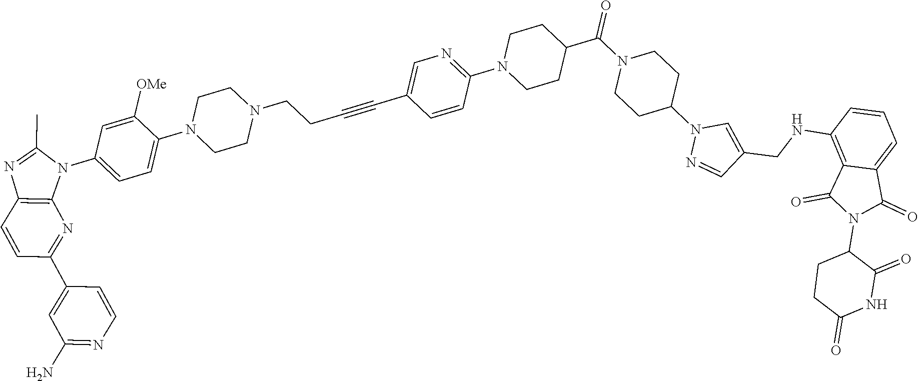 [SMILES: COc1cc(-n2c(C)nc3ccc(-c4ccnc(N)c4)nc32)ccc1N1CCN(CCC#Cc2ccc(N3CCC(C(=O)N4CCC(n5cc(CNc6cccc7c6C(=O)N(C6CCC(=O)NC6=O)C7=O)cn5)CC4)CC3)nc2)CC1]

### 4-(((1-(1-(1-(5-(4-(4-(4-(5-(2-aminopyridin-4-yl)-2-methyl-3H-imidazo[4,5-b]pyridin-3-yl)-2-methoxyphenyl)piperazin-1-yl) but-1-yn-1-yl)pyridin-2-yl)piperidine-4-carbonyl)piperidin-4-yl)-1H-pyrazol-4-yl)methyl)amino)-2-(2,6-dioxopiperidin-3-yl)isoindoline-1,3-dione (38)

DMF (1.70 mL) and triethylamine (22.4 mg, 0.22 mmol, 5.0 eq.) were added sequentially to a dry 5 mL MWV equipped with a magnetic stir bar under an inert atmosphere containing 4-(3-(4-(4-(but-3-yn-1-yl)piperazin-1-yl)-3-methoxyphenyl)-2-methyl-3H-imidazo[4,5-b]pyridin-5-yl)pyridin-2-amine (38e) (20.7 mg, 44.2 μmol, 1.0 eq.), 2-(2,6-dioxopiperidin-3-yl)-4-(((1-(1-(1-(5-iodopyridin-2-yl)piperidine-4-carbonyl)piperidin-4-yl)-1H-pyrazol-4-yl)methyl)amino)isoindoline-1,3-dione (D7) (33.2 mg, 44.2 μmol, 1.0 eq.), PdCl₂ (dppf) (4.20 mg, 5.75 μmol, 0.13 eq.) and copper (I) iodide (2.10 mg, 11.1 μmol, 0.25 eq.). This reaction mixture was stirred at room temperature for 24 hours where it was concentrated under reduced pressure and purified by flash column chromatography to give 4-(((1-(1-(1-(5-(4-(4-(4-(5-(2-aminopyridin-4-yl)-2-methyl-3H-imidazo[4,5-b]pyridin-3-yl)-2-methoxyphenyl)piperazin-1-yl) but-1-yn-1-yl)pyridin-2-yl)piperidine-4-carbonyl)piperidin-4-yl)-1H-pyrazol-4-yl)methyl)amino)-2-(2,6-dioxopiperidin-3-yl)isoindoline-1,3-dione (38) (18.1 mg, 16.6 μmol, 37% yield), a yellow solid. LCMS (ESI) C₆₀H₆₃N₁₅O₆ requires 1089.5, found 1090.5 (M/Z+H).

### Example 39. Synthesis of 4-(((1-(1-(1-(5-(3-(3-(4-(4-(5-(2-aminopyridin-4-yl)-2-methyl-3H-imidazo[4,5-b]pyridin-3-yl)-2,6-difluorophenyl)piperazin-1-yl)azetidin-1-yl)prop-1-yn-1-yl)pyridin-2-yl)piperidine-4-carbonyl)piperidin-4-yl)-1H-pyrazol-4-yl)methyl)amino)-2-(2,6-dioxopiperidin-3-yl)isoindoline-1,3-dione (39)

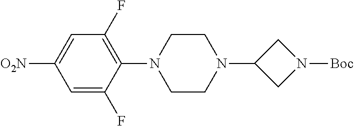 [SMILES: CC(C)(C)OC(=O)N1CC(N2CCN(c3c(F)cc([N+](=O)[O-])cc3F)CC2)C1]

### tert-butyl 3-(4-(2,6-difluoro-4-nitrophenyl)piperazin-1-yl)azetidine-1-carboxylate (39a)

To a solution of 1-(2,6-difluoro-4-nitrophenyl)piperazine (349 mg, 1.43 mmol, 1.0 eq.) and tert-butyl 3-oxoazetidine-1-carboxylate (492 mg, 2.87 mmol, 2.0 eq.) in 1,2-dichloroethane (9.58 mL) was added acetic acid (0.20 mL, 3.59 mmol, 2.5 eq.). After stirring for 30 minutes, sodium triacetoxyborohydride (609 mg, 2.87 mmol, 2.0 eq.) was added and the reaction mixture was stirred at room temperature overnight. The mixture was quenched with a saturated solution of sodium bicarbonate and extracted with ethyl acetate. The organic layers were washed with brine, dried over sodium sulfate, and concentrated under reduced pressure. The residue was purified by flash column chromatography using a hexanes/ethyl acetate gradient to give tert-butyl 3-(4-(2,6-difluoro-4-nitrophenyl)piperazin-1-yl)azetidine-1-carboxylate (39a) (507 mg, 1.27 mmol, 62% yield, 70% wt.), a red oil. LCMS (ESI) C₁₈H₂₄F₂N₄O₄ requires 398.1, found 399.4 (M/Z+H).

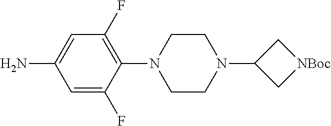 [SMILES: CC(C)(C)OC(=O)N1CC(N2CCN(c3c(F)cc(N)cc3F)CC2)C1]

tert-butyl 3-(4-(4-amino-2,6-difluorophenyl)piperazin-1-yl)azetidine-1-carboxylate (39b) To a 8 mL MWV equipped with a magnetic stir bar containing tert-butyl 3-(4-(2,6-difluoro-4-nitrophenyl)piperazin-1-yl)azetidine-1-carboxylate (39a) (350 mg, 615 μmol, 70% wt., 1.0 eq.) in ethanol (4.28 mL) and water (1.07 mL) was added ammonium chloride (19.7 mg, 0.36 mmol, 0.6 eq.) and was stirred at 40° C. Iron (206 mg, 3.69 mmol, 6.0 eq.) was then added and heated to 90° C. and stirred for 2 hours. After cooling to room temperature, the reaction mixture was filtered through celite and partitioned between DCM and saturated sodium bicarbonate. The aqueous phase was extracted with DCM (3×) and the combined organic phases were recombined and dried over sodium sulfate. The residue was redissolved in chloroform and concentrated under reduced pressure (3×) to give tert-butyl 3-(4-(4-amino-2,6-difluorophenyl)piperazin-1-yl)azetidine-1-carboxylate (39b) (222 mg, 0.54 mmol, 88% yield, 90% Purity), an amber oil.

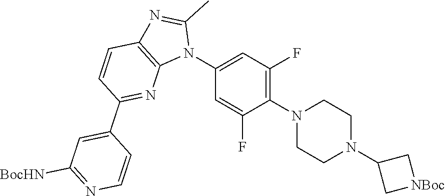 [SMILES: Cc1nc2ccc(-c3ccnc(NC(=O)OC(C)(C)C)c3)nc2n1-c1cc(F)c(N2CCN(C3CN(C(=O)OC(C)(C)C)C3)CC2)c(F)c1]

### tert-butyl 3-(4-(4-(5-(2-((tert-butoxycarbonyl)amino)pyridin-4-yl)-2-methyl-3H-imidazo[4,5-b]pyridin-3-yl)-2,6-difluorophenyl)piperazin-1-yl)azetidine-1-carboxylate (39c)

Tert-butyl 3-(4-(4-amino-2,6-difluorophenyl)piperazin-1-yl)azetidine-1-carboxylate (39b) (203 mg, 0.55 mmol, 1.0 eq.), tert-butyl (5-acetamido-6-chloro-[2,4′-bipyridin]-2′-yl)carbamate (A5) (200 mg, 0.55 mmol, 1.0 eq.), potassium phosphate (351 mg, 1.65 mmol, 3.0 eq.), dicyclohexyl(2′,4′, 6′-triisopropyl-[1,1′-biphenyl]-2-yl)phosphane (26.3 mg, 55.1 μmol, 0.10 eq.), and Pd₂(dba)₃ (10.1 mg, 11.0 μmol, 0.02 eq.) were added to a flame-dried 5 ml MWV containing a flame dried stir bar. The MWV was capped and purged with argon. Degassed t-BuOH (1.38 mL) was added to the MWV and the reaction mixture was stirred at 90° C. for 24 hours. The reaction mixture was cooled to room temperature, diluted with DCM, filtered through celite, and washed with a solution of 5% MeOH in DCM. The filtrate was concentrated under reduced pressure and purified by flash column chromatography to give-butyl 3-(4-(4-(5-(2-((tert-butoxycarbonyl)amino)pyridin-4-yl)-2-methyl-3H-imidazo[4,5-b]pyridin-3-yl)-2,6-difluorophenyl)piperazin-1-yl)azetidine-1-carboxylate (39c) (190 mg, 0.28 mmol, 50% yield), a brown solid.

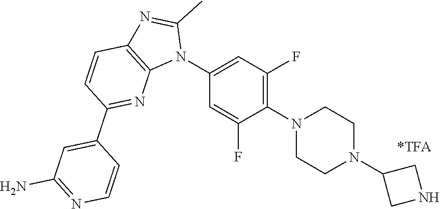 [SMILES: Cc1nc2ccc(-c3ccnc(N)c3)nc2n1-c1cc(F)c(N2CCN(C3CNC3)CC2)c(F)c1]

### 4-(3-(4-(4-(azetidin-3-yl)piperazin-1-yl)-3,5-difluorophenyl)-2-methyl-3H-imidazo[4,5-b]pyridin-5-yl)pyridin-2-amine, Trifluoroacetic acid (39d)

HCl (2.60 mL, 10.4 mmol, 4.0 M in 1,4-dioxane, 35.0 eq.) was added to a 5 mL MWV equipped with a magnetic stir bar containing tert-butyl 3-(4-(4-(5-(2-((tert-butoxycarbonyl)amino)pyridin-4-yl)-2-methyl-3H-imidazo[4,5-b]pyridin-3-yl)-2,6-difluorophenyl)piperazin-1-yl)azetidine-1-carboxylate (39c) (201 mg, 0.29 mmol, 1.0 eq.) The reaction mixture was stirred for 24 hours at room temperature where it was then concentrated under reduced pressure and extracted with DCM. The organic layer was washed with a saturated solution of sodium bicarbonate then dried over magnesium sulfate, filtered, and concentrated under reduced pressure to give the mono-boc (Boc-2-aminopyridine). Trifluoroacetic acid (0.67 mL, 8.71 mmol, 25.0 eq.) and DCM (1.85 mL) were added and the reaction mixture was stirred for 3 hours where it was then triturated with hexanes and concentrated under reduced pressure to give 4-(3-(4-(4-(azetidin-3-yl)piperazin-1-yl)-3,5-difluorophenyl)-2-methyl-3H-imidazo[4,5-b]pyridin-5-yl)pyridin-2-amine, Trifluoroacetic acid (39d) (176 mg, 0.29 mmol, 85% yield), a black solid. LCMS (ESI) C₂₅H₂₆F₂N₈ requires 476.22, found 477.1 (M/Z+H).

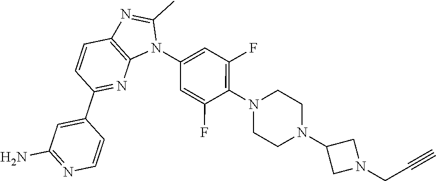 [SMILES: C#CCN1CC(N2CCN(c3c(F)cc(-n4c(C)nc5ccc(-c6ccnc(N)c6)nc54)cc3F)CC2)C1]

### 4-(3-(3,5-difluoro-4-(4-(1-(prop-2-yn-1-yl)azetidin-3-yl)piperazin-1-yl)phenyl)-2-methyl-3H-imidazo[4,5-b]pyridin-5-yl)pyridin-2-amine (39e)

Propargyl bromide (9.7 μL, 0.11 mmol, 0.95 eq.) was added to a 5 mL MWV equipped with a magnetic stir bar containing 4-(3-(4-(4-(azetidin-3-yl)piperazin-1-yl)-3,5-difluorophenyl)-2-methyl-3H-imidazo[4,5-b]pyridin-5-yl)pyridin-2-amine, Trifluoroacetic acid (39d) (71 mg, 0.12 mmol, 1.0 eq.) and potassium carbonate (53 mg, 0.12 mmol, 1.0 eq.) in DMF (1.09 mL). The reaction mixture was stirred at room temperature for 16 hours where it was then washed with a saturated solution of sodium bicarbonate (5 mL) and extracted with DCM (3×15 mL). The organic layers were recombined, dried over sodium sulfate, filtered, and concentrated under reduced pressure, The crude residue was purified by flash column chromatography using a DCM/MeOH gradient to afford 4-(3-(3,5-difluoro-4-(4-(1-(prop-2-yn-1-yl)azetidin-3-yl)piperazin-1-yl)phenyl)-2-methyl-3H-imidazo[4,5-b]pyridin-5-yl)pyridin-2-amine (39e) (11 mg, 21 μmol, 18% yield). LCMS (ESI) C₂₈H₂₈F₂N₈ requires 514.24, found 515.1 (M/Z+H).

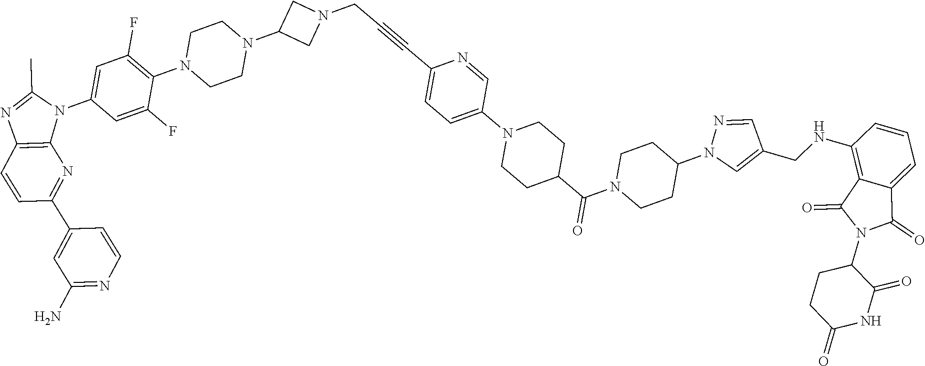 [SMILES: Cc1nc2ccc(-c3ccnc(N)c3)nc2n1-c1cc(F)c(N2CCN(C3CN(CC#Cc4ccc(N5CCC(C(=O)N6CCC(n7cc(CNc8cccc9c8C(=O)N(C8CCC(=O)NC8=O)C9=O)cn7)CC6)CC5)cn4)C3)CC2)c(F)c1]

### 4-(((1-(1-(1-(5-(3-(3-(4-(4-(5-(2-aminopyridin-4-yl)-2-methyl-3H-imidazo[4,5-b]pyridin-3-yl)-2,6-difluorophenyl)piperazin-1-yl)azetidin-1-yl)prop-1-yn-1-yl)pyridin-2-yl)piperidine-4-carbonyl)piperidin-4-yl)-1H-pyrazol-4-yl)methyl)amino)-2-(2,6-dioxopiperidin-3-yl)isoindoline-1,3-dione (39)

A flame dried 5 mL MWV equipped with a magnetic stir bar containing 2-(2,6-dioxopiperidin-3-yl)-4-(((1-(1-(1-(5-iodopyridin-2-yl)piperidine-4-carbonyl)piperidin-4-yl)-1H-pyrazol-4-yl)methyl)amino)isoindoline-1,3-dione (34i) (16 mg, 21 μmol, 1.0 eq.), 4-(3-(3,5-difluoro-4-(4-(1-(prop-2-yn-1-yl)azetidin-3-yl)piperazin-1-yl)phenyl)-2-methyl-3H-imidazo[4,5-b]pyridin-5-yl)pyridin-2-amine (23e) (11 mg, 21 μmol, 1.1 eq.), copper (I) iodide (0.49 mg, 2.6 μmol, 0.12 eq.), and PdCl₂ (dppf) (1.3 mg, 1.7 μmol, 0.08 eq.) was added DMF (0.86 mL) and triethylamine (8.9 μL, 64 μmol, 3.0 eq.) sequentially and was run at room temperature under an atmosphere of argon for 16 hours where it was concentrated under reduced pressure and purified by flash column chromatography to give 4-(((1-(1-(1-(5-(3-(3-(4-(4-(5-(2-aminopyridin-4-yl)-2-methyl-3H-imidazo[4,5-b]pyridin-3-yl)-2,6-difluorophenyl)piperazin-1-yl)azetidin-1-yl)prop-1-yn-1-yl)pyridin-2-yl)piperidine-4-carbonyl)piperidin-4-yl)-1H-pyrazol-4-yl)methyl)amino)-2-(2,6-dioxopiperidin-3-yl)isoindoline-1,3-dione (39). LCMS (ESI) C₆₁H₆₂F₂N₁₆O₅ requires 1136.50, found 569.4 (M/2+H).

### Example 40. Synthesis of 4-(((1-(1-(1-(5-(3-(6-(4-(5-(2-aminopyridin-4-yl)-2-methyl-3H-imidazo[4,5-b]pyridin-3-yl)-2,6-difluorophenyl)-2,6-diazaspiro[3.3]heptan-2-yl)prop-1-yn-1-yl)pyridin-2-yl)piperidine-4-carbonyl)piperidin-4-yl)-1H-pyrazol-4-yl)methyl)amino)-2-(2,6-dioxopiperidin-3-yl)isoindoline-1,3-dione (40)

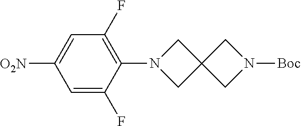 [SMILES: CC(C)(C)OC(=O)N1CC2(C1)CN(c1c(F)cc([N+](=O)[O-])cc1F)C2]

### tert-butyl 6-(2,6-difluoro-4-nitrophenyl)-2,6-diazaspiro[3.3]heptane-2-carboxylate (40a)

To a 5 mL MWV equipped with a magnetic stir bar containing tert-butyl 2,6-diazaspiro[3.3]heptane-2-carboxylate (221 mg, 1.11 mmol, 1.0 eq.), potassium carbonate (461 mg, 3.34 mmol, 3.0 eq.), in DMF (1.11 mL) was added 1,2,3-trifluoro-5-nitrobenzene (0.13 mL, 1.0 eq. 1.11 mmol). The reaction mixture was stirred at 90° C. for 24 hours where the resulting mixture was cooled to room temperature and extracted with ethyl acetate (2×20 mL) and washed with water. The organic layer was then washed with water and brine (5×10 mL) then the organic layer was dried over sodium sulfate, filtered, and concentrated under reduced pressure. The crude residue was purified by flash column chromatography to give tert-butyl 6-(2,6-difluoro-4-nitrophenyl)-2,6-diazaspiro[3.3]heptane-2-carboxylate (40a) (339 mg, 0.95 mmol, 85% yield), an orange solid. LCMS (ESI) C₁₆H₁₉F₂N₃₀₄ requires 355.1, found 356 (M/Z+H).

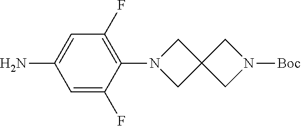 [SMILES: CC(C)(C)OC(=O)N1CC2(C1)CN(c1c(F)cc(N)cc1F)C2]

### tert-butyl 6-(4-amino-2,6-difluorophenyl)-2,6-diazaspiro[3.3]heptane-2-carboxylate (40b)

To a 8 mL MWV equipped with a magnetic stir bar containing tert-butyl 6-(2,6-difluoro-4-nitrophenyl)-2,6-diazaspiro[3.3]heptane-2-carboxylate (40a) (339 mg, 0.95 mmol, 1.0 eq.) in ethanol (6.6 mL) and water (1.6 mL) was added ammonium chloride (30 mg, 0.57 mmol 0.6 eq.) and was stirred at 40° C. Iron (320 mg, 5.72 mmol, 6.0 eq.) was then added and heated to 90° C. and stirred for 4 hours. After cooling to room temperature, the reaction mixture was filtered and partitioned between DCM and saturated sodium bicarbonate. The aqueous phase was extracted with DCM (3×) and the combined organic phases were dried over sodium sulfate. The residue was redissolved in chloroform and concentrated under reduced pressure (3×) to give tert-butyl 6-(4-amino-2,6-difluorophenyl)-2,6-diazaspiro[3.3]heptane-2-carboxylate (40b) (267 mg, 821 μmol, 86%, yield), a gray solid. LCMS (ESI) C₁₆H₂₁F₂N₃₀₂ requires 325.16, found 325.9 (M/Z+H).

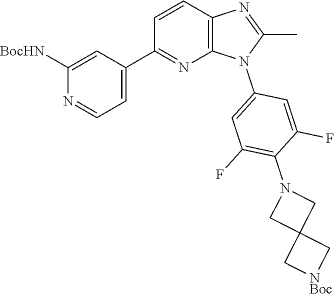 [SMILES: Cc1nc2ccc(-c3ccnc(NC(=O)OC(C)(C)C)c3)nc2n1-c1cc(F)c(N2CC3(CN(C(=O)OC(C)(C)C)C3)C2)c(F)c1]

### tert-butyl 6-(4-(5-(2-((tert-butoxycarbonyl)amino)pyridin-4-yl)-2-methyl-3H-imidazo[4,5-b]pyridin-3-yl)-2,6-difluorophenyl)-2,6-diazaspiro[3.3]heptane-2-carboxylate (40c)

Potassium phosphate (523 mg, 2.46 mmol, 3.0 eq.), tert-butyl (5-acetamido-6-chloro-[2,4′-bipyridin]-2′-yl)carbamate (A5) (298 mg, 821 μmol, 1.0e q.), Pd₂dba₃ (15.0 mg, 16.4 μmol, 0.02 eq.), dicyclohexyl(2′,4′,6′-triisopropyl-[1,1′-biphenyl]-2-yl)phosphane (39.2 mg, 82.1 μmol, 0.10 eq.), and tert-butyl 6-(4-amino-2,6-difluorophenyl)-2,6-diazaspiro[3.3]heptane-2-carboxylate (40b) (267 mg, 821 μmol, 1.0 eq.) were added to a flame-dried 5 mL MWV containing a flame dried stir bar. The MWV was capped and purged with argon. Degassed t-BuOH (2.05 mL) was added to the MWV and heated to 90° C. in an oil bath. The reaction mixture was allowed to cool to room temperature and diluted with dichloromethane. The diluted mixture was filtered through Celite with the aid of dichloromethane and 5% MeOH in DCM. The filtrate was concentrated under reduced pressure and purified by flash column chromatography to give tert-butyl 6-(4-(5-(2-((tert-butoxycarbonyl)amino)pyridin-4-yl)-2-methyl-3H-imidazo[4,5-b]pyridin-3-yl)-2,6-difluorophenyl)-2,6-diazaspiro[3.3]heptane-2-carboxylate (40c) (400 mg, 0.63 mmol, 76% yield), a light-brown solid. LCMS (ESI) C₃₃H₃₇F₂N₇₀₄ requires 633.2, found 634.0 (M/Z+H).

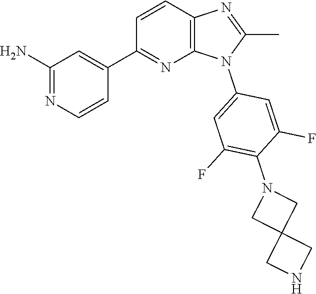 [SMILES: Cc1nc2ccc(-c3ccnc(N)c3)nc2n1-c1cc(F)c(N2CC3(CNC3)C2)c(F)c1]

### 4-(3-(3,5-difluoro-4-(2,6-diazaspiro[3.3]heptan-2-yl)phenyl)-2-methyl-3H-imidazo[4,5-b]pyridin-5-yl)pyridin-2-amine (40d)

A 4M solution of HCl in 1,4-dioxane (6.31 mL, 25.2 mmol, 40.0 eq.) was added to a 20 mL MWV equipped with a magnetic stir bar containing tert-butyl 6-(4-(5-(2-((tert-butoxycarbonyl)amino)pyridin-4-yl)-2-methyl-3H-imidazo[4,5-b]pyridin-3-yl)-2,6-difluorophenyl)-2,6-diazaspiro[3.3]heptane-2-carboxylate (40c) (400 mg, 0.63 mmol, 1.0 eq.). The reaction mixture was stirred overnight and concentrated under reduced pressure. LCMS showed that the mono-boc intermediate was still present so dichloromethane (4.00 mL) and trifluoroacetic acid (2.00 mL, 26.0 mmol, 44.0 eq.) were added. The reaction mixture was stirred overnight where it was then concentrated under reduced pressure. The crude residue was extracted with DCM and washed with a saturated solution of sodium bicarbonate. The organic layer was dried over magnesium sulfate, filtered, and concentrated under reduced pressure to give 4-(3-(3,5-difluoro-4-(2,6-diazaspiro[3.3]heptan-2-yl)phenyl)-2-methyl-3H-imidazo[4,5-b]pyridin-5-yl)pyridin-2-amine (40d), a brown solid and was used in the next step without further purification. LCMS (ESI) C₂₃H₂₁F₂N₇ requires 433.1, found 470 (M/Z+HCl).

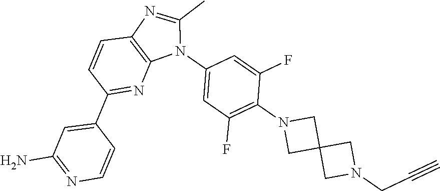 [SMILES: C#CCN1CC2(C1)CN(c1c(F)cc(-n3c(C)nc4ccc(-c5ccnc(N)c5)nc43)cc1F)C2]

### 4-(3-(3,5-difluoro-4-(6-(prop-2-yn-1-yl)-2,6-diazaspiro[3.3]heptan-2-yl)phenyl)-2-methyl-3H-imidazo[4,5-b]pyridin-5-yl)pyridin-2-amine, HCl (40e)

3-bromoprop-1-yne (18 μL, 0.17 mmol, 1.05 eq. 80% wt.) was added to a 5 mL MWV equipped with a magnetic stir bar containing 4-(3-(3,5-difluoro-4-(2,6-diazaspiro[3.3]heptan-2-yl)phenyl)-2-methyl-3H-imidazo[4,5-b]pyridin-5-yl)pyridin-2-amine, HCl (40d) (90 mg, 0.16 mmol, 1.0 eq.) and potassium carbonate (57 mg, 0.41 mmol, 2.5 eq.) in DMF (3.3 mL) The reaction mixture was stirred at room temperature for 16 hours where it was then quenched with a saturated solution of sodium bicarbonate and extracted with DCM. The organic layers were recombined, dried over sodium sulfate, and concentrated under reduced pressure. The crude residue was purified by automated flash column chromatography using a DCM/MeOH gradient to afford 4-(3-(3,5-difluoro-4-(6-(prop-2-yn-1-yl)-2,6-diazaspiro[3.3]heptan-2-yl)phenyl)-2-methyl-3H-imidazo[4,5-b]pyridin-5-yl)pyridin-2-amine, HCl (40e) (25 mg, 49 μmol, 30% yield, eluting at 7% MeOH in DCM). LCMS (ESI) C₂₆H₂₃F₂N₇ requires 471.1, found 508.1 (M/Z+HCl).

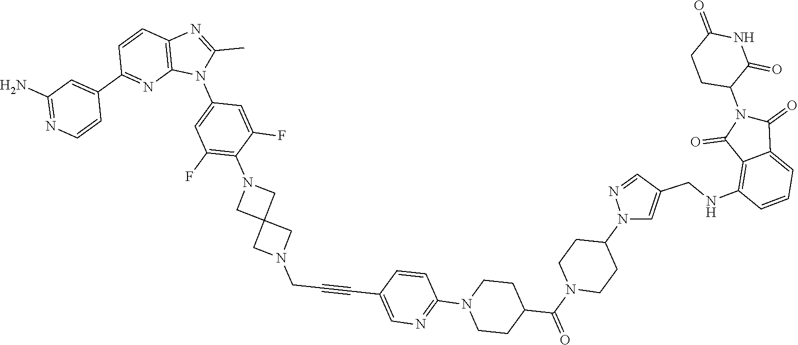 [SMILES: Cc1nc2ccc(-c3ccnc(N)c3)nc2n1-c1cc(F)c(N2CC3(CN(CC#Cc4ccc(N5CCC(C(=O)N6CCC(n7cc(CNc8cccc9c8C(=O)N(C8CCC(=O)NC8=O)C9=O)cn7)CC6)CC5)nc4)C3)C2)c(F)c1]

### 4-(((1-(1-(1-(5-(3-(6-(4-(5-(2-aminopyridin-4-yl)-2-methyl-3H-imidazo[4,5-b]pyridin-3-yl)-2,6-difluorophenyl)-2,6-diazaspiro[3.3]heptan-2-yl)prop-1-yn-1-yl)pyridin-2-yl)piperidine-4-carbonyl)piperidin-4-yl)-1H-pyrazol-4-yl)methyl)amino)-2-(2,6-dioxopiperidin-3-yl)isoindoline-1,3-dione (40)

DMF (2.0 mL) and triethylamine (21 μL, 0.15 mmol, 3.0 eq.) were added sequentially to a dry 5 mL MWV equipped with a magnetic stir bar containing 4-(3-(3,5-difluoro-4-(6-(prop-2-yn-1-yl)-2,6-diazaspiro[3.3]heptan-2-yl)phenyl)-2-methyl-3H-imidazo[4,5-b]pyridin-5-yl)pyridin-2-amine, HCl (40e) (25 mg, 49 μmol, 1.0 eq.), 3-(4-(((1-(1-(1-(5-iodopyridin-2-yl)piperidine-4-carbonyl)piperidin-4-yl)-1H-pyrazol-4-yl)methyl)amino)-1-oxoisoindolin-2-yl)piperidine-2,6-dione (D7) (36 mg, 49 μmol, 1.0 eq.), PdCl₂ (dppf) (2.9 mg, 3.9 μmol, 0.08 eq.) and copper (I) iodide (1.1 mg, 5.9 μmol, 0.12 eq.). This was stirred for 24 hours at room temperature where it was concentrated under reduced pressure and purified by flash column chromatography to give 4-(((1-(1-(1-(5-(3-(6-(4-(5-(2-aminopyridin-4-yl)-2-methyl-3H-imidazo[4,5-b]pyridin-3-yl)-2,6-difluorophenyl)-2,6-diazaspiro[3.3]heptan-2-yl)prop-1-yn-1-yl)pyridin-2-yl)piperidine-4-carbonyl)piperidin-4-yl)-1H-pyrazol-4-yl)methyl)amino)-2-(2,6-dioxopiperidin-3-yl)isoindoline-1,3-dione (40) (3.1 mg, 2.8 μmol, 5% yield, eluting at 20% MeOH in DCM), a yellow solid. LCMS (ESI) C₅₉H₅₇F₂N₁₅O₅ requires 1093.4, found 1130.3 (M/Z+HCl).

### Example II

This example describes a screening assay and KDElect assay for the DYRK1A/B PROTACs described herein (see, Table 2).

The Flp-In T-REx 293 Cell Line and the pcDNA5/FRT/TO vector (Invitrogen) were used to generate stable cell clones for tetracyclin-inducible expression of DYRK1A and DYRK1B. These cells contain a single stably integrated Flp recognition target (FRT) site, which allows for site-specific integration of the expression cassette by the Flp (Flippase) recombinase. DYRK1 constructs were designed to contain a C-terminal HiBiT tag for quantitative detection of HiBiT-tagged proteins in cellular lysates with the help a split luciferase assay (Nano-Glo HiBiT Lytic Detection System, Promega). To monitor the targeted degradation of DYRK1A or DYRK1B, cells were seeded in 96-well plates (10,000 cells/per well) and treated with doxycyclineinal concentration 2 μg/ml) to induce DYRK1 expression. Compounds with presumed PROTAC activity were administered 24 h after seeding. After further incubation for 24 h, cells were grossly and microscopically inspected for overt toxicity. Cells were lysed in 100 μL ice-cold passive lysis buffer (PLB, Promega) by orbital shaking (30 min on ice) before lysates were centrifuged in PCR tube (20 min 20,000 g, RT). A 10 μl aliquot of the supernatant was subjected to the HiBiT lytic assay. Luminescence was quantified in an Orion Microplate luminometer (Berthold Detection System, Germany). All compounds were tested in duplicate or triplicate wells (technical replicates) and at least two independent experiments. Relative degradation was calculated by normalization to vehicle-treated control samples.

KDs were obtained from Eurofins who used the following procedure: Kinase tagged T7 phase strains were prepared in an E. coli host derived from the BL21 strain. E. coli were grown to log-phase and infected with T7 phage and incubated with shaking at 32° C. until lysis. The lysates were centrifuged and filtered to remove cell debris. The remaining kinases were produced in HEK-293 cells and subsequently tagged with DNA for qPCR detection. Streptavidin-coated magnetic beads were treated with biotinylated small molecule ligands for 30 minutes at room temperature to generate affinity resins for kinase assays. The liganded beads were blocked with excess biotin and washed with locking buffer (SeaBlock (Pierce), 1% BSA, 0.05% Tween 20, 1 mM DTT) to remove unbound ligand and to reduce non-specific binding. Binding reactions were assembled by combining kinases, liganded affinity beads, and test compounds in 1× binding buffer (20% SeaBlock, 0.17× PBS, 0.05% Tween 20, 6 mM DTT). Test compounds were prepared as 111X stocks in 100% DMSO. Kds were determined using a 11-poin 3-fold compound dilution series with three DMSO control points. All compounds for Kd measurements are distributed by acoustic transfer (non-contact dispensing) in 100% DMSO. The compounds were then diluted directly into the assays such that the final concentration of DMSO was 0.9%. All reactions performed in polypropylene 384-well plate. Each was a final volume of 0.02 mL. The assay plates were incubated at room temperature with shaking for 1 hour and the affinity beads were washed with wash buffer (1×PBS, 0.05% Tween 20). The beads were then resuspended in elution buffer (1X PBS, 0.05% Tween 20, 0.5 UM nonbiotinylated affinity ligand) and incubated at room temperature with shaking for 30 minutes. The kinase concentration in the eluates was measured by qPCR.

TABLE 2

| | |
|---|---|
| Relative Degradation | Binding |
| at 300 nM | Affinity (Kd) |

| Cpd. No. | DYRK1A | DYRK1B | DYRK1A | DYRK1B |
|---|---|---|---|---|
| 1 | + | + | +++ | +++ |
| 2 | + | + | +++ | +++ |
| 3 | + | + | +++ | +++ |
| 4 | + | + | +++ | +++ |
| 5 | ++ | + | +++ | +++ |
| 6 | ++ | + | +++ | +++ |
| 7 | ++ | ++ | +++ | +++ |
| 8 | + | — | +++ | — |
| 9 | + | — | +++ | — |
| 10 | ++ | + | +++ | ++ |
| 11 | + | — | ++ | ++ |
| 12 | ++ | — | +++ | +++ |
| 13 | + | — | ++ | ++ |
| 14 | + | — | +++ | +++ |
| 15 | + | — | +++ | — |
| 16 | ++ | — | +++ | +++ |
| 17 | + | — | +++ | — |
| 18 | +++ | +++ | +++ | ++ |
| 19 | +++ | +++ | ++ | ++ |
| 20 | ++ | + | +++ | ++ |
| 21 | ++ | + | ++ | + |
| 22 | ++ | + | — | — |
| 23 | +++ | +++ | +++ | +++ |
| 24 | +++ | + | ++ | ++ |
| 25 | +++ | ++ | +++ | +++ |
| 26 | +++ | ++ | — | — |
| 27 | +++ | + | — | — |
| 28 | +++ | — | +++ | ++ |
| 29 | — | — | — | — |
| 30 | — | — | — | — |
| 31 | + | + | — | — |
| 32 | +++ | +++ | ++ | ++ |
| 33 | +++ | — | — | — |
| 34 | — | — | +++ | +++ |
| 35 | — | — | +++ | ++ |
| 36 | — | — | +++ | +++ |
| 37 | — | — | +++ | ++ |
| 38 | — | — | +++ | ++ |
| 39 | — | — | +++ | +++ |
| 40 | — | — | +++ | ++ |

Relative degradation of DYRK1A at 300 nM: +++ = >60%, ++ = 30-60%, + = <30%, + = <30%, ++ = 30-60%, +++ = >60%.
Relative degradation of DYRK1B at 300 nM: +++ = >50%, ++ = 30-50%, + = <30%.
Kd DYRK1A/B: +++ = <10 nM, ++ = 10-100 nM, + = 100-1000 nM.
— = No data available

### INCORPORATION BY REFERENCE

The entire disclosure of each of the patent documents and scientific articles referred to herein is incorporated by reference for all purposes. The following references are herein incorporated by reference in their entireties:

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

What is claimed is:

1. A bifunctional compound comprising the chemical structure:
TBM-L-ULM or TBM-ULM, wherein:
TBM is a binding moiety for one or more of DYRK1A, DYRK1B, DYRK2, DYRK3, CLK1, CLK2, CLK3, CLK4, and HASPIN;
L is a bond or a chemical linker that covalently compounds the TBM and the ULM; and
ULM is an E3 ubiquitin ligase binding moiety.

2. The bifunctional compound of claim 1, wherein TBM is a small molecule inhibitor of one or more of DYRK1A, DYRK1B, DYRK2, DYRK3, CLK1, CLK2, CLK3, CLK4, and HASPIN.

3. The bifunctional compound of claim 2, wherein the small molecule inhibitor of one or more of DYRK1A, DYRK1B, DYRK2, DYRK3, CLK1, CLK2, CLK3, CLK4, and HASPIN is selected from: 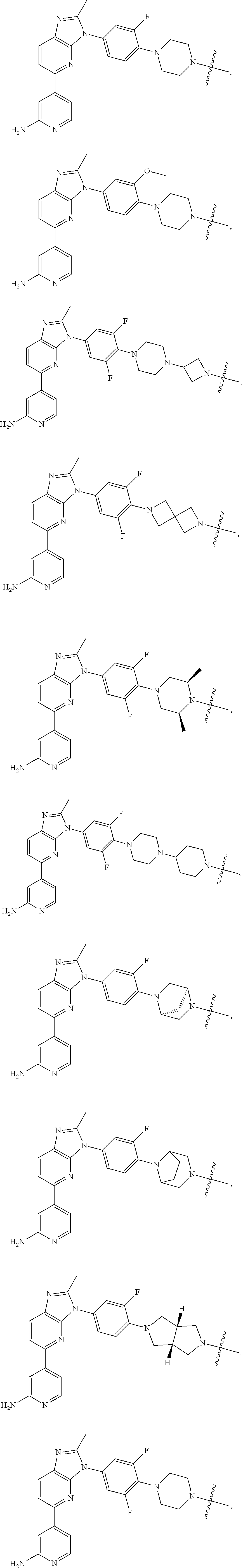 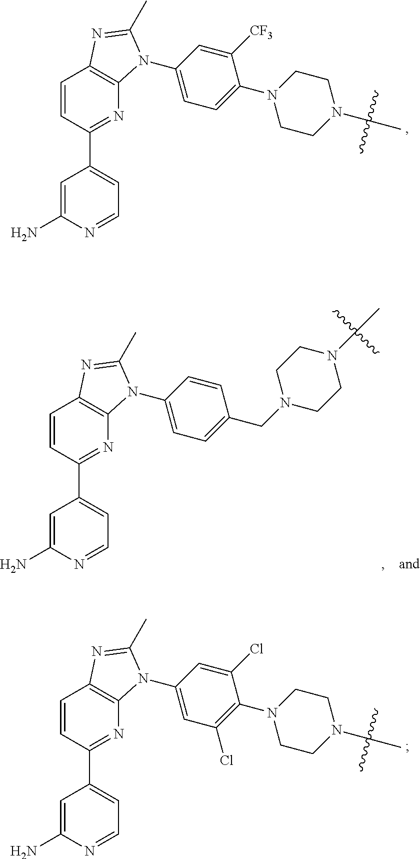 [SMILES: *N1CCN(c2ccc(-n3c(C)nc4ccc(-c5ccnc(N)c5)nc43)cc2C(F)(F)F)CC1 ; *N1CCN(Cc2ccc(-n3c(C)nc4ccc(-c5ccnc(N)c5)nc43)cc2)CC1 ; *N1CCN(c2c(Cl)cc(-n3c(C)nc4ccc(-c5ccnc(N)c5)nc43)cc2Cl)CC1]
wherein “ [symbol] ” indicates the location where the small molecule covalently couples with the linker.

4. The bifunctional compound of claim 1, wherein the ULM covalently couples with the linker at any location within the ULM.

5. The bifunctional compound of claim 1, wherein the ULM is selected from: VHL 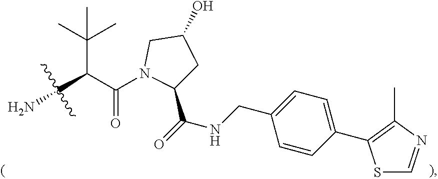
cereblon 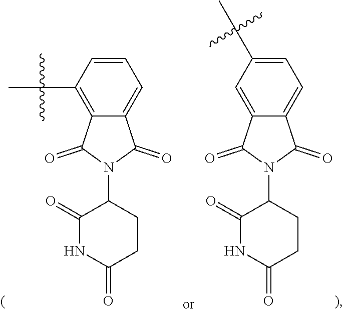
2-(2,6-dioxopiperidin-3-yl)-5-fluoroisoindoline-1,3-dione 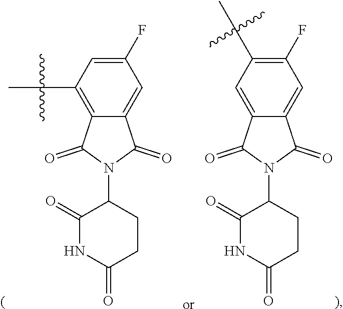
3-(1-oxoisoindolin-2-yl)piperidine-2,6-dione 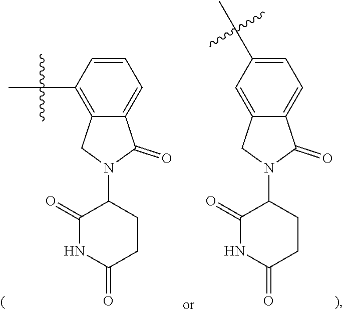
2-(1-methyl-2,6-dioxopiperidin-3-yl)isoindoline-1,3-dione 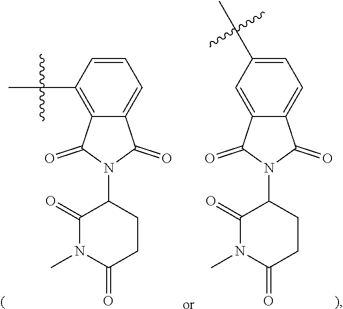
and 3-phenylpiperidine-2,6-dione 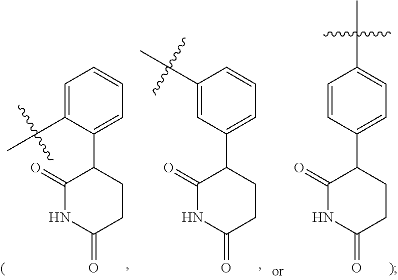 [SMILES: *c1ccccc1C1CCC(=O)NC1=O ; *c1cccc(C2CCC(=O)NC2=O)c1 ; *c1ccc(C2CCC(=O)NC2=O)cc1]
wherein “ [symbol] ” indicates the location where the ULM covalently couples with the linker.

6. The bifunctional compound of claim 1, wherein the linker is represented by one of the following: 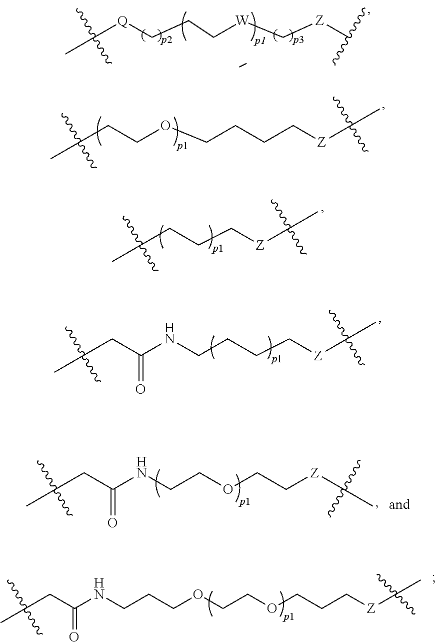
or an enantiomer, diastereomer, or stereoisomer thereof, wherein p1 is an integer selected from 0 to 12; p2 is an integer selected from 0 to 12; p3 is an integer selected from 1 to 6; each W is independently absent, CH₂, O, S, NH, or NR⁸; Z is absent, CH₂, O, NH, or NR⁸; each R⁸ is independently C₁-C₃ alkyl; and Q is absent or CH₂C(O)NH.

7. The bifunctional compound of claim 1, wherein the linker is selected from: the linker is selected from:

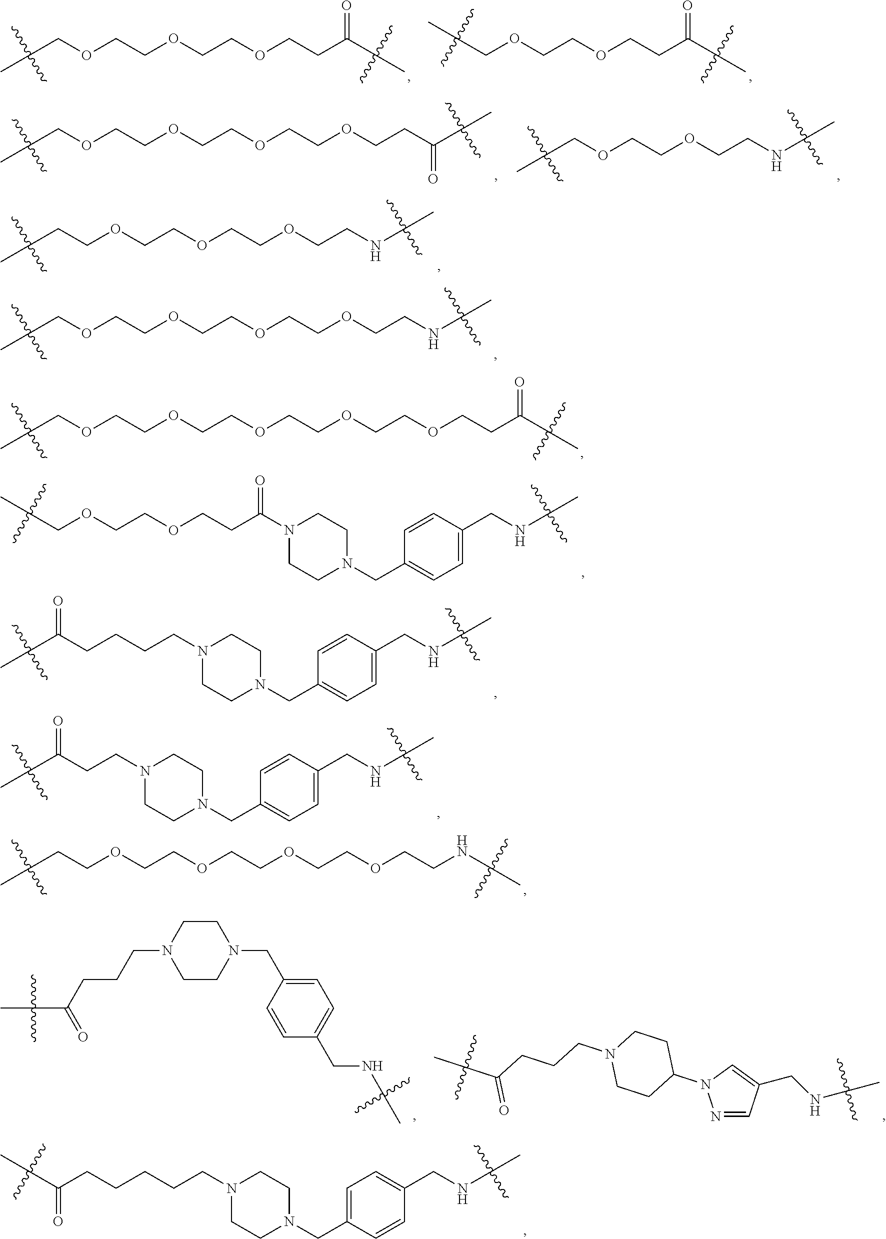

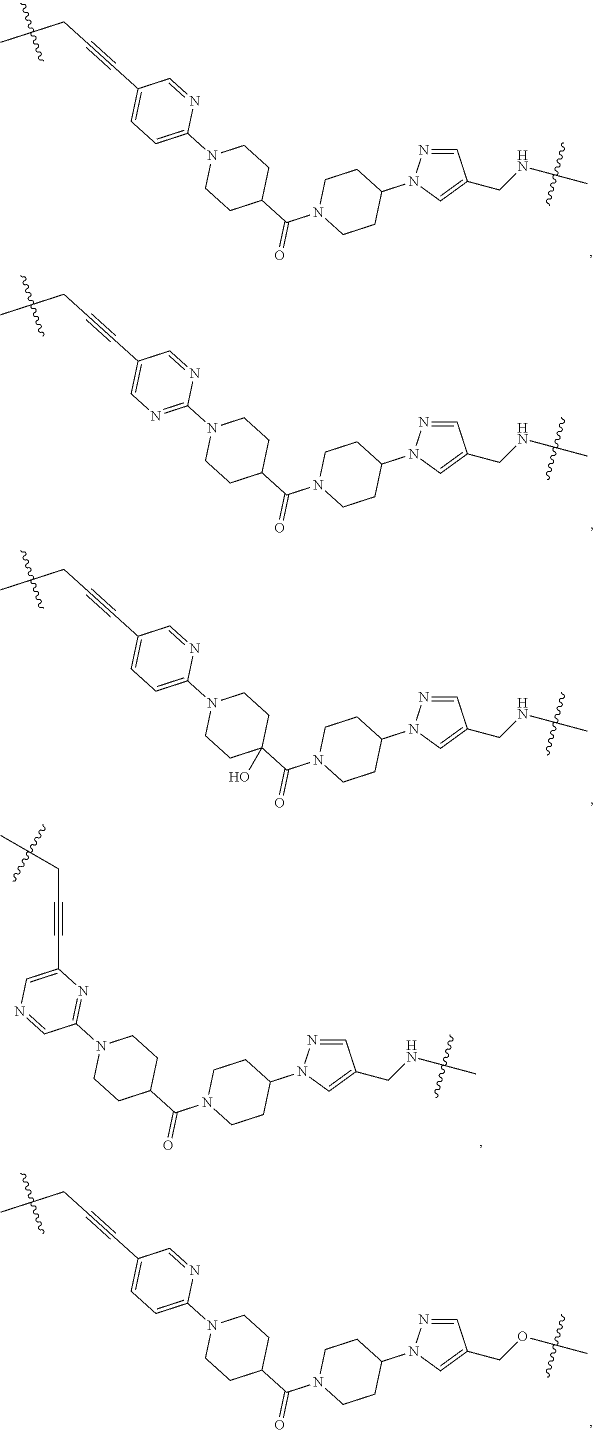 [SMILES: *CC#Cc1ccc(N2CCC(C(=O)N3CCC(n4cc(CN*)cn4)CC3)CC2)nc1 ; *CC#Cc1cnc(N2CCC(C(=O)N3CCC(n4cc(CN*)cn4)CC3)CC2)nc1 ; *CC#Cc1ccc(N2CCC(O)(C(=O)N3CCC(n4cc(CN*)cn4)CC3)CC2)nc1 ; *CC#Cc1cncc(N2CCC(C(=O)N3CCC(n4cc(CN*)cn4)CC3)CC2)n1 ; *CC#Cc1ccc(N2CCC(C(=O)N3CCC(n4cc(CO*)cn4)CC3)CC2)nc1]

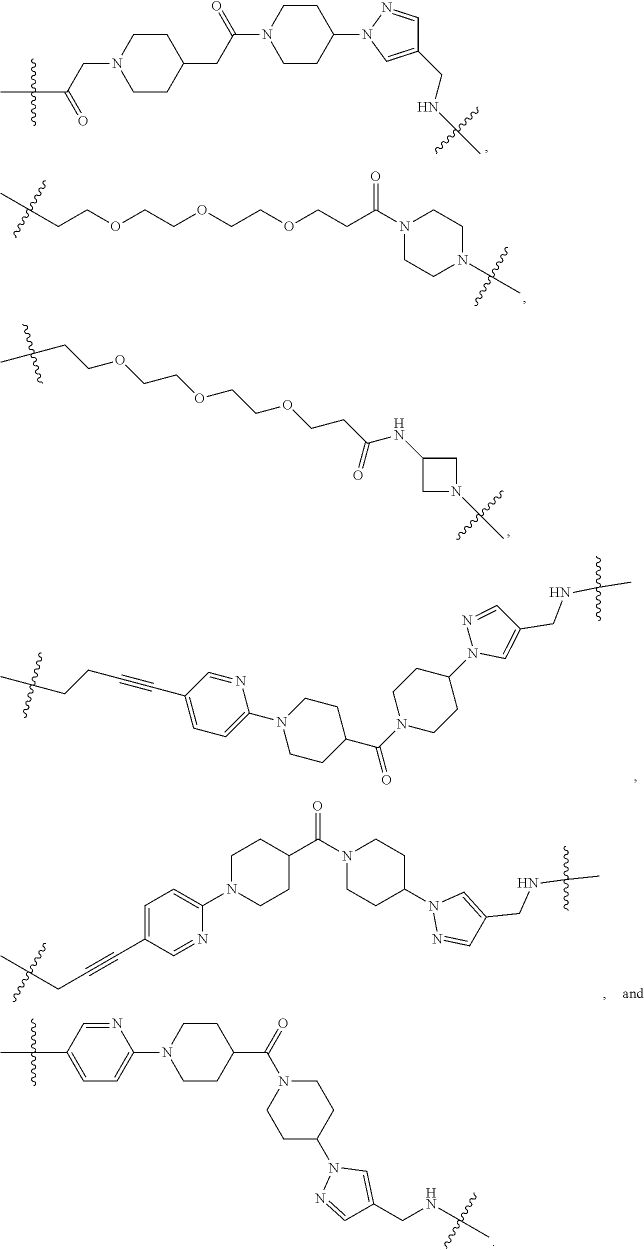

8. The bifunctional compound of claim 1,
wherein TBM-L-ULM is represented by TBM-A-B-modified-cereblon;
wherein A is represented by 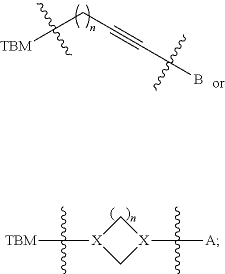
wherein B is represented by 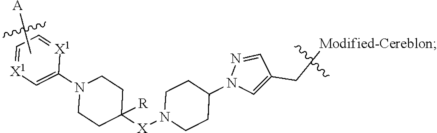
wherein modified-cereblon is selected from: 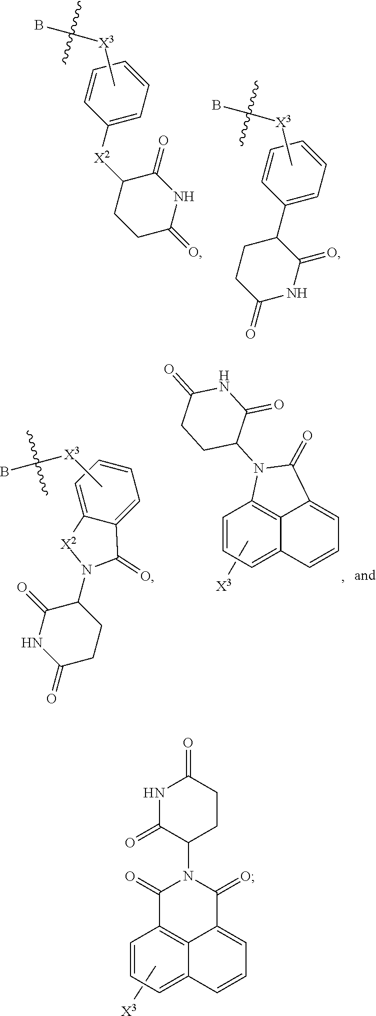
wherein n is 0, 1 or 2;
wherein R is OH or H;
wherein X is CH₂ or CO;
wherein each X¹ is either N or CH;
wherein X² is CO, CH₂, or NH;
wherein X³ is NH or O.

9. The bifunctional compound of claim 1, wherein the bifunctional compound is selected from:

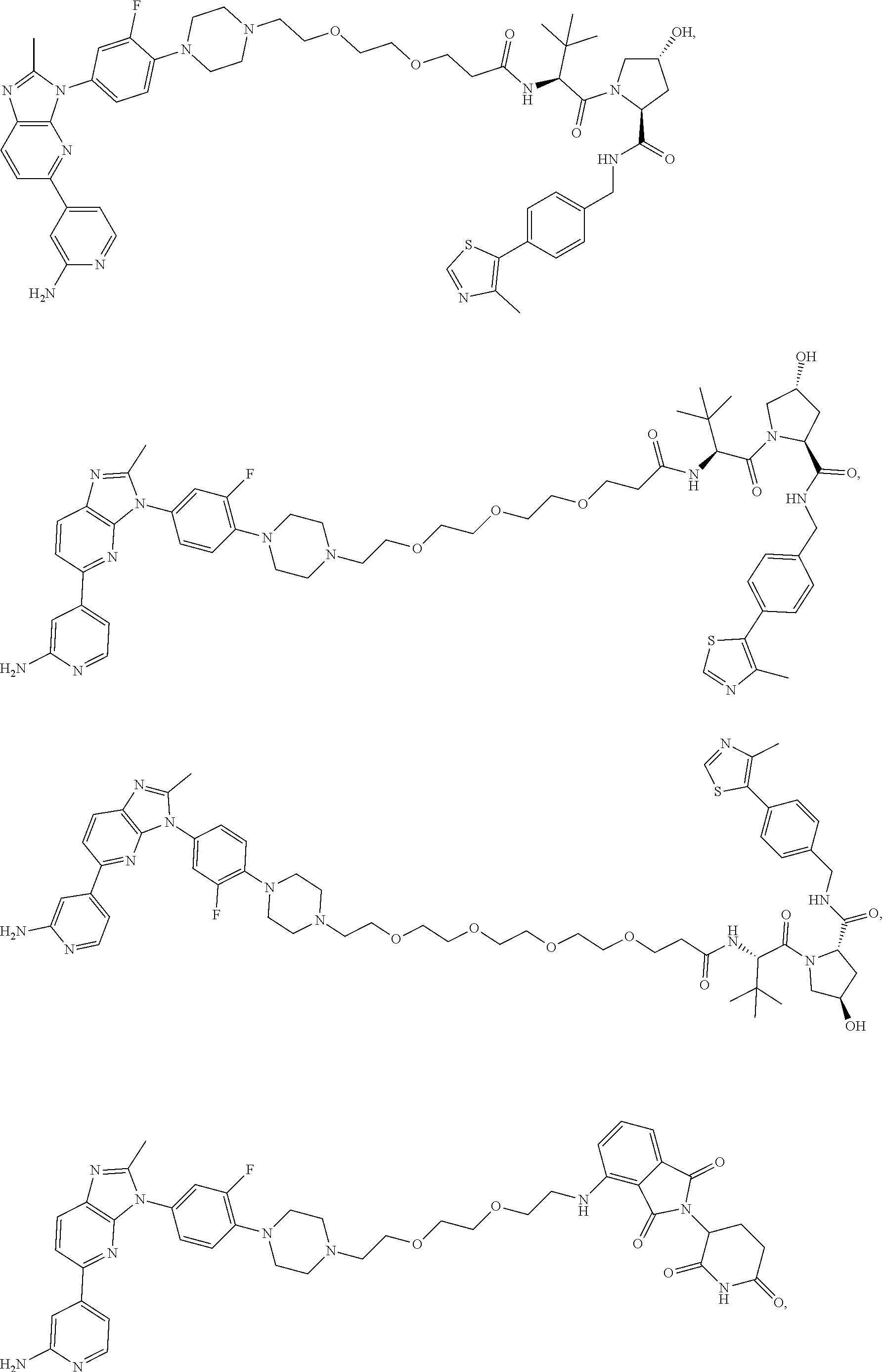

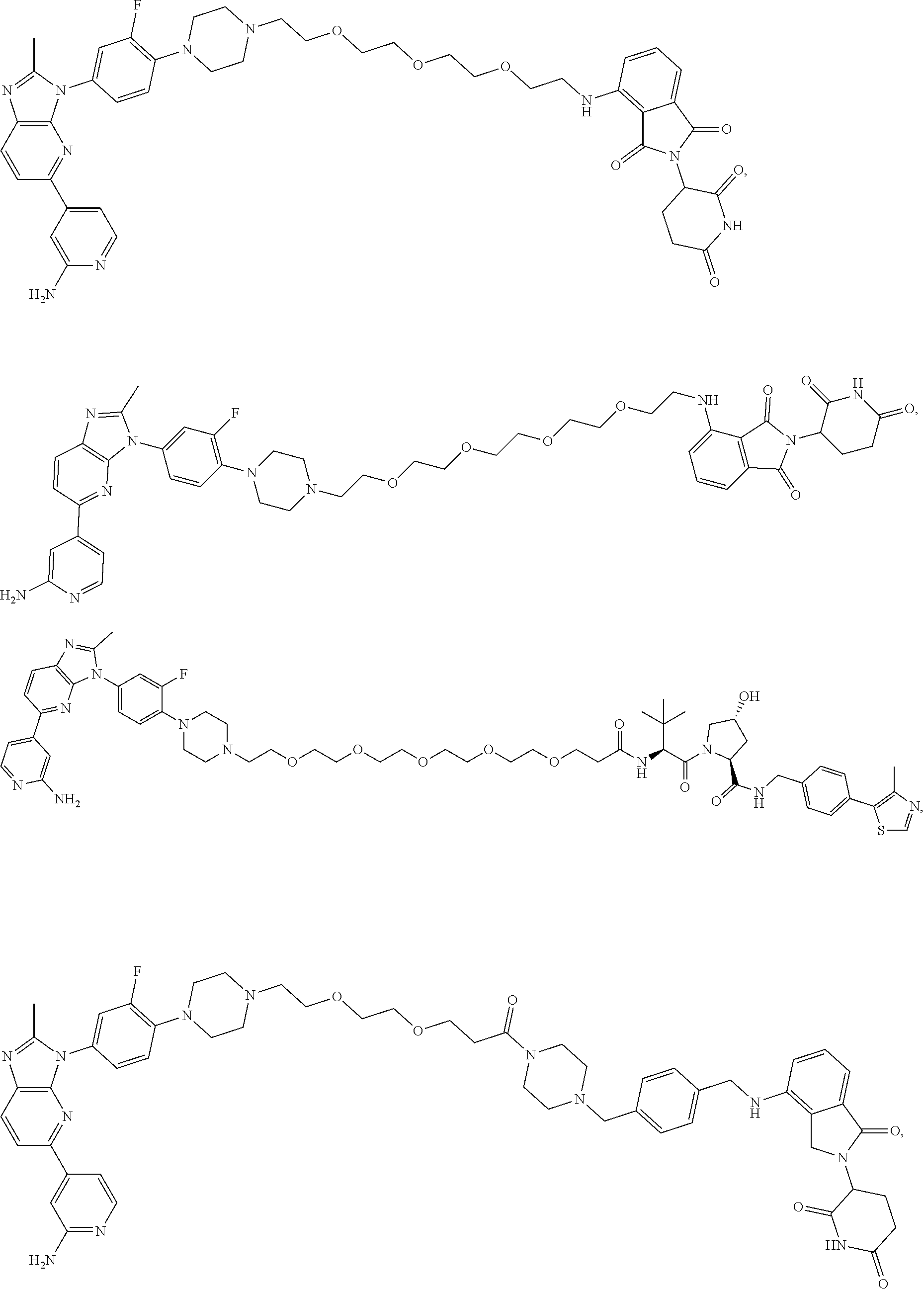 [SMILES: Cc1nc2ccc(-c3ccnc(N)c3)nc2n1-c1ccc(N2CCN(CCOCCOCCOCCNc3cccc4c3C(=O)N(C3CCC(=O)NC3=O)C4=O)CC2)c(F)c1 ; Cc1nc2ccc(-c3ccnc(N)c3)nc2n1-c1ccc(N2CCN(CCOCCOCCOCCOCCNc3cccc4c3C(=O)N(C3CCC(=O)NC3=O)C4=O)CC2)c(F)c1 ; Cc1ncsc1-c1ccc(CNC(=O)[C@@H]2C[C@@H](O)CN2C(=O)[C@@H](NC(=O)CCOCCOCCOCCOCCOCCN2CCN(c3ccc(-n4c(C)nc5ccc(-c6ccnc(N)c6)nc54)cc3F)CC2)C(C)(C)C)cc1 ; Cc1nc2ccc(-c3ccnc(N)c3)nc2n1-c1ccc(N2CCN(CCOCCOCCC(=O)N3CCN(Cc4ccc(CNc5cccc6c5CN(C5CCC(=O)NC5=O)C6=O)cc4)CC3)CC2)c(F)c1]

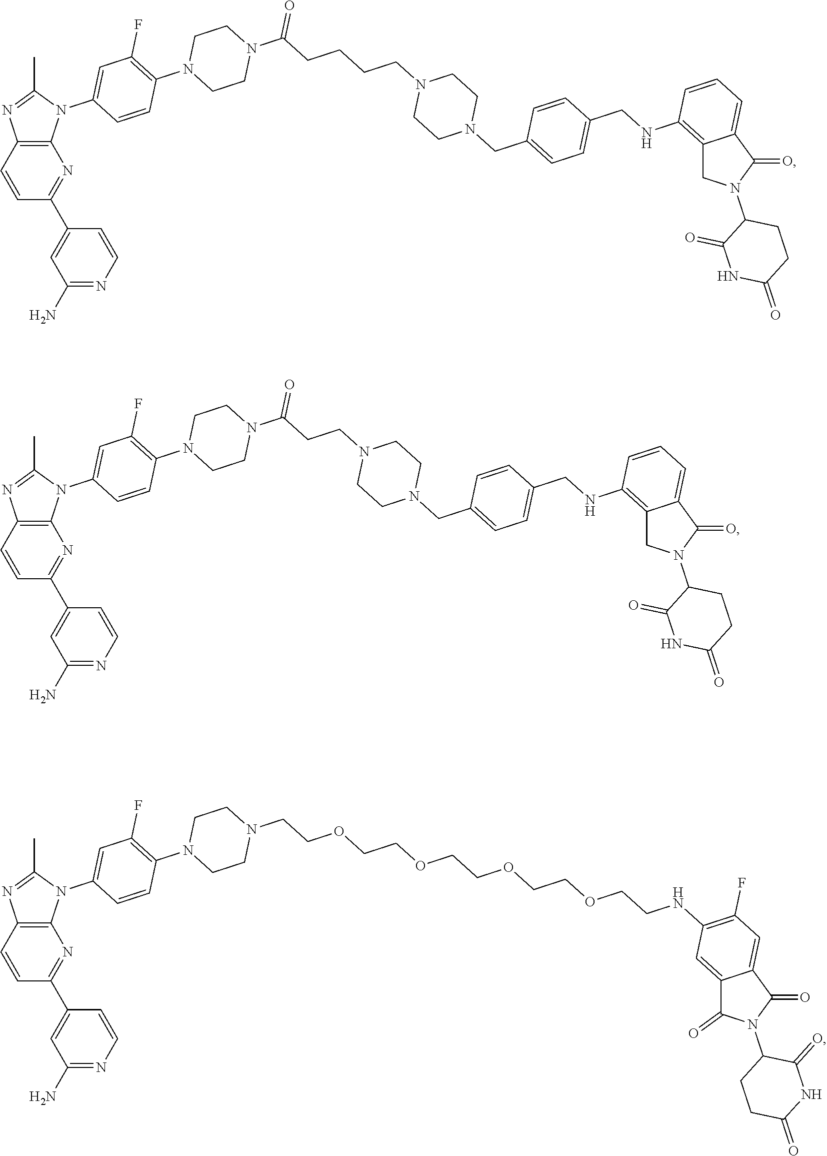 [SMILES: Cc1nc2ccc(-c3ccnc(N)c3)nc2n1-c1ccc(N2CCN(C(=O)CCCCN3CCN(Cc4ccc(CNc5cccc6c5CN(C5CCC(=O)NC5=O)C6=O)cc4)CC3)CC2)c(F)c1 ; Cc1nc2ccc(-c3ccnc(N)c3)nc2n1-c1ccc(N2CCN(C(=O)CCN3CCN(Cc4ccc(CNc5cccc6c5CN(C5CCC(=O)NC5=O)C6=O)cc4)CC3)CC2)c(F)c1 ; Cc1nc2ccc(-c3ccnc(N)c3)nc2n1-c1ccc(N2CCN(CCOCCOCCOCCOCCNc3cc4c(cc3F)C(=O)N(C3CCC(=O)NC3=O)C4=O)CC2)c(F)c1]

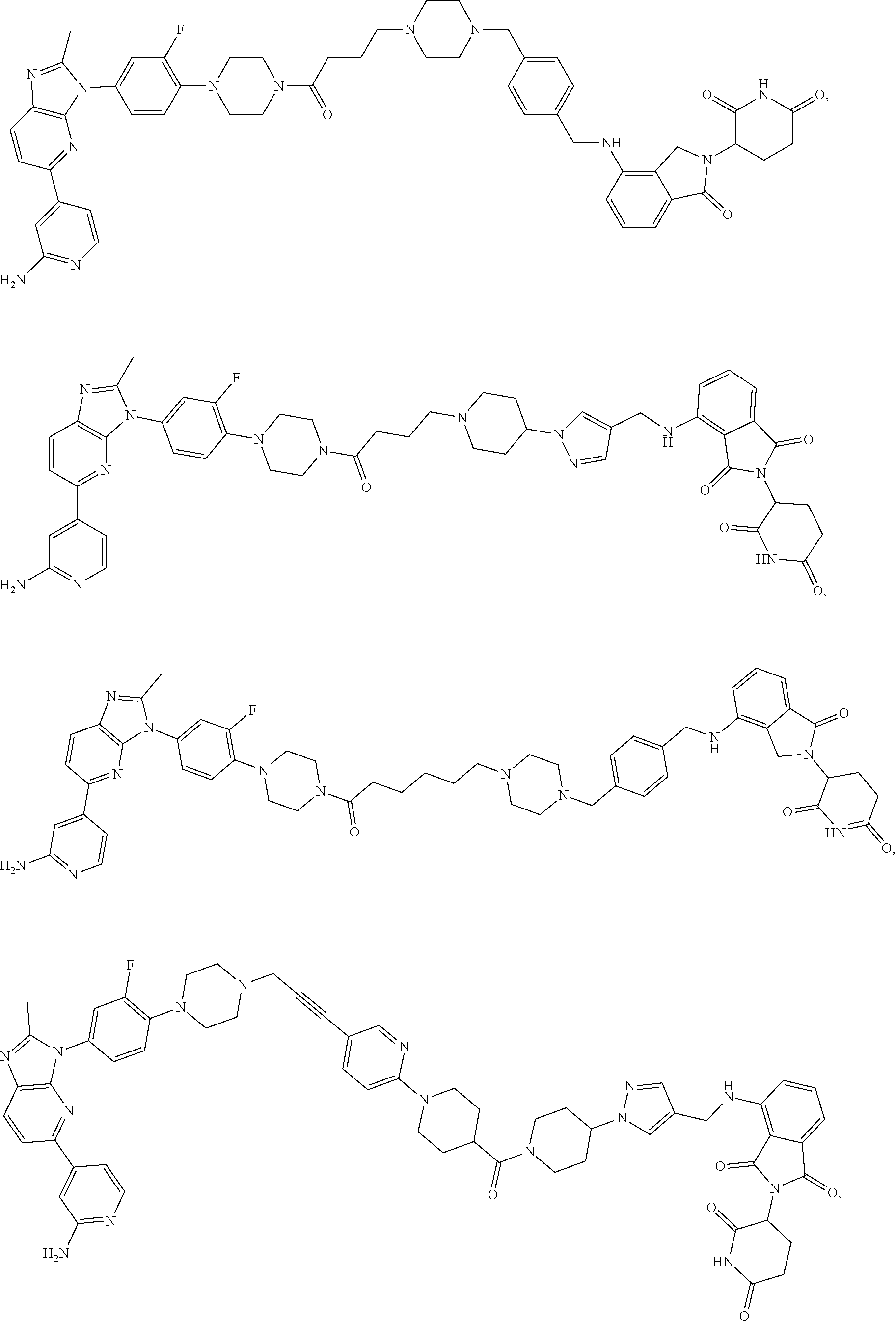

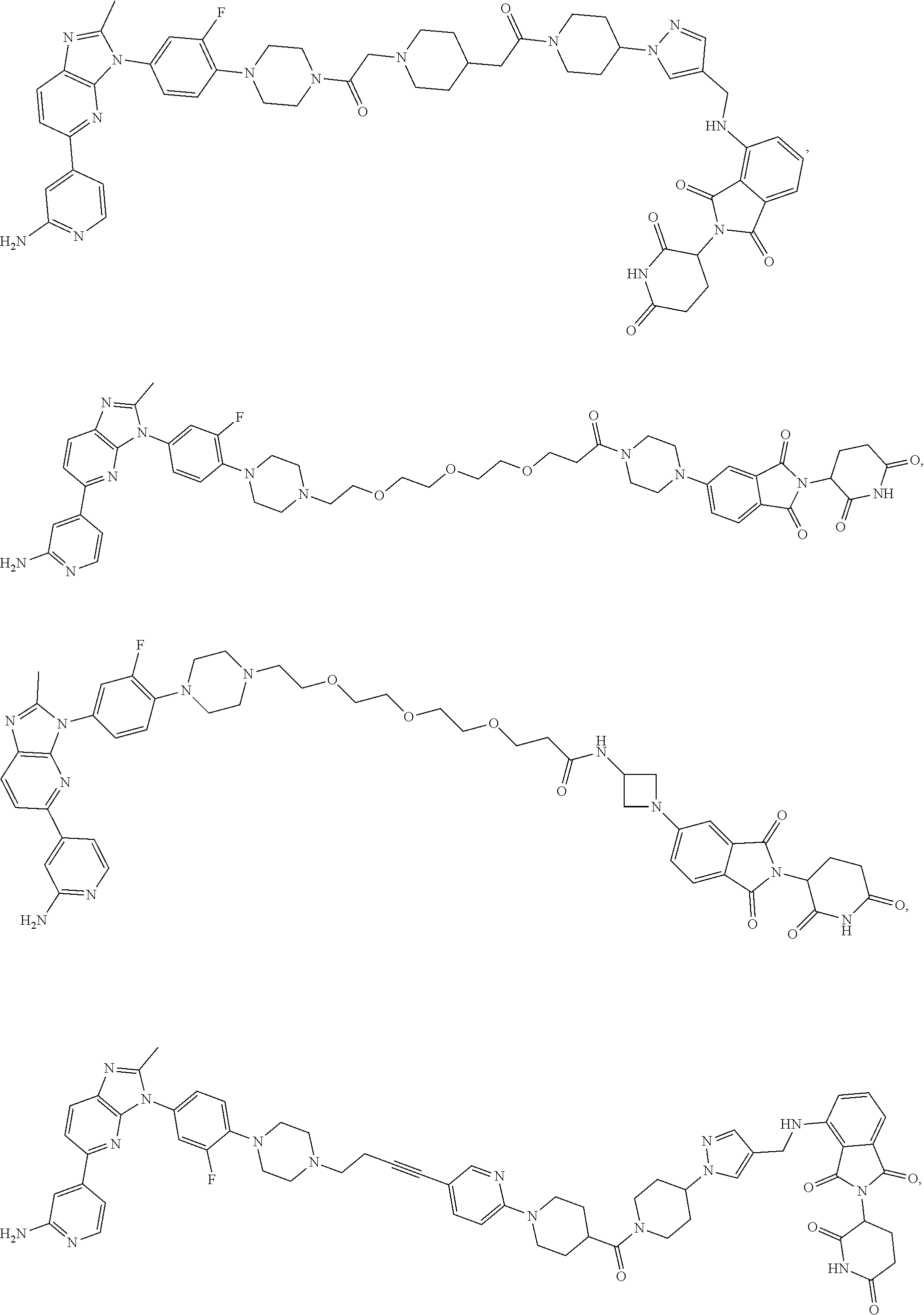

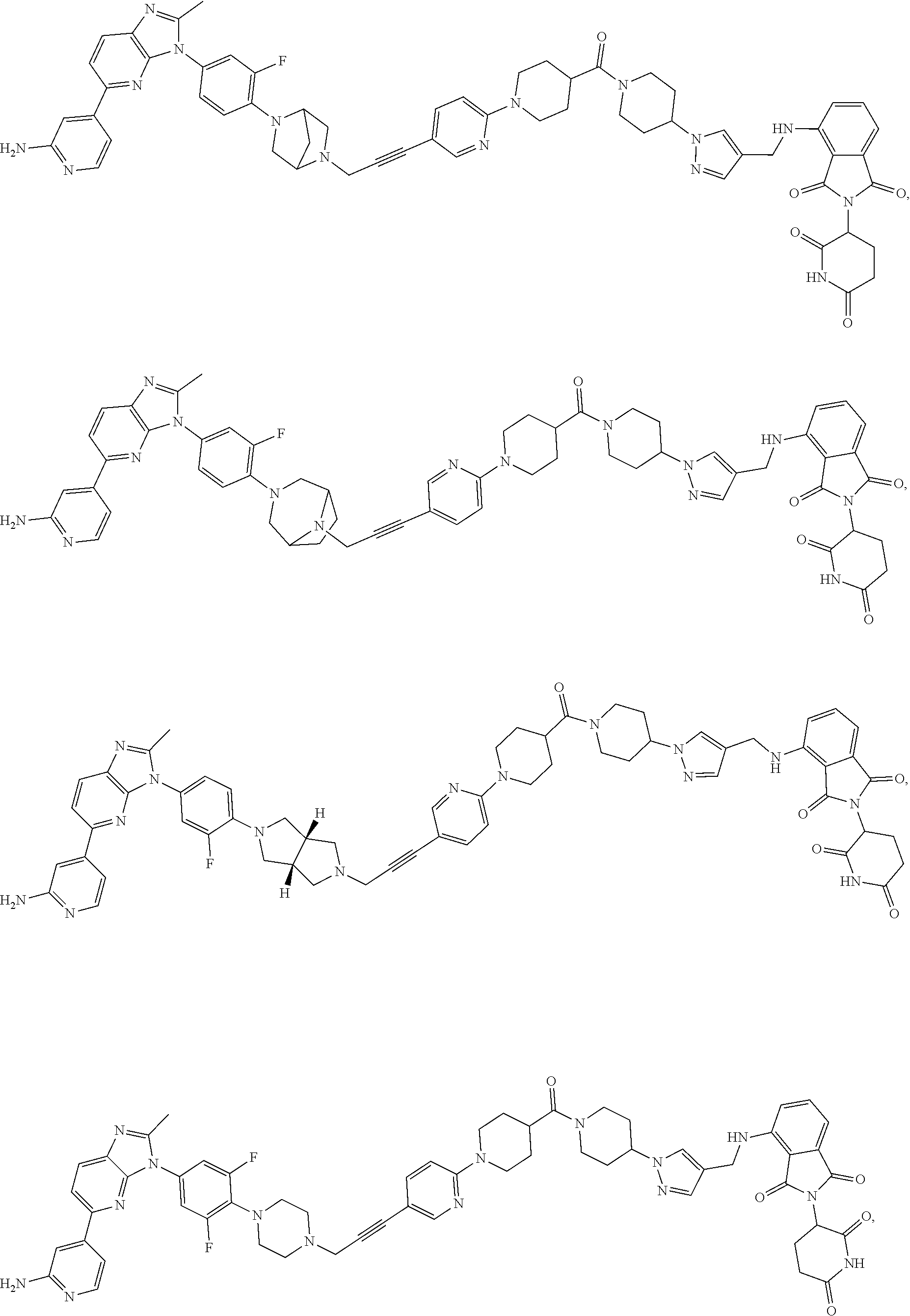

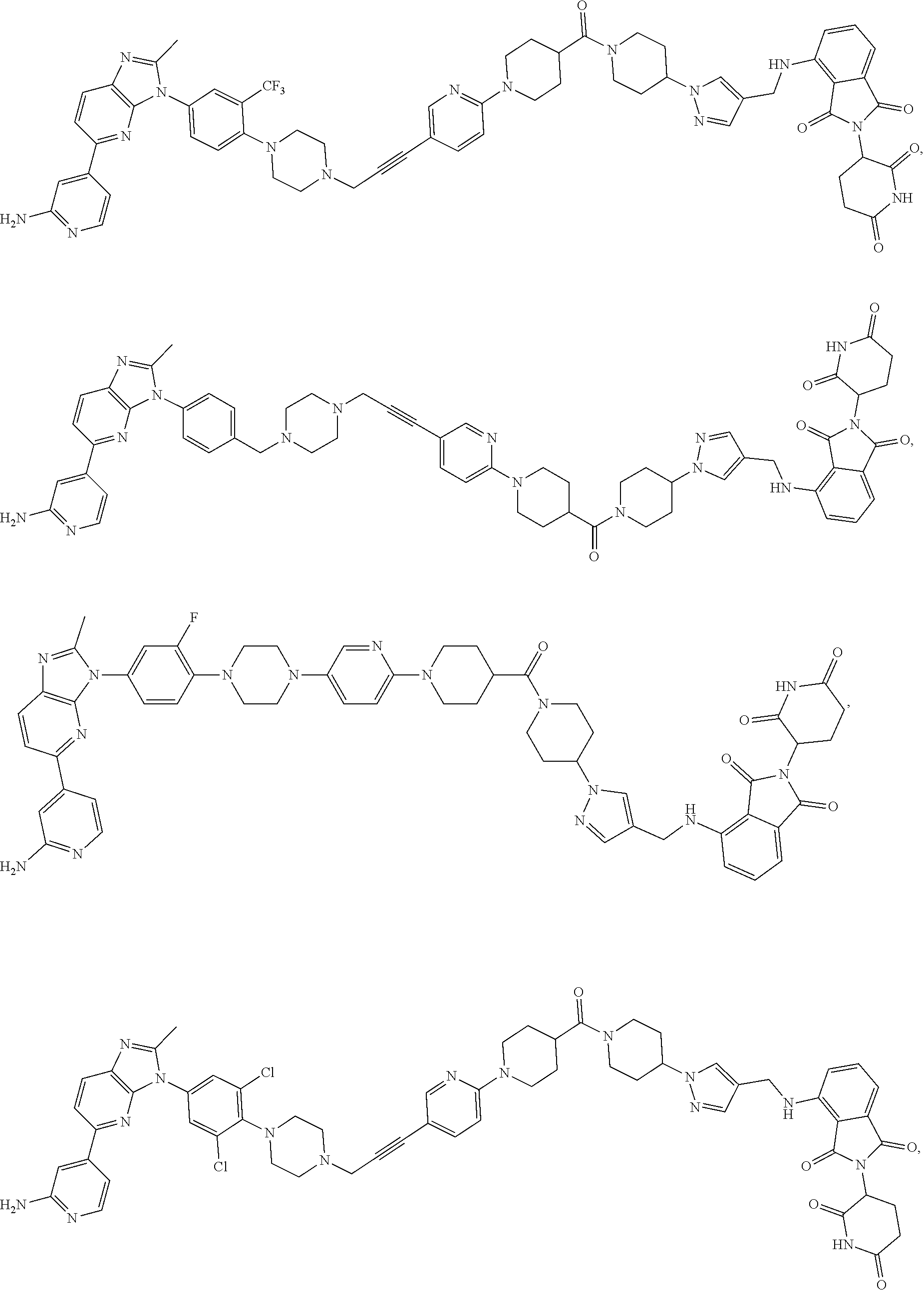

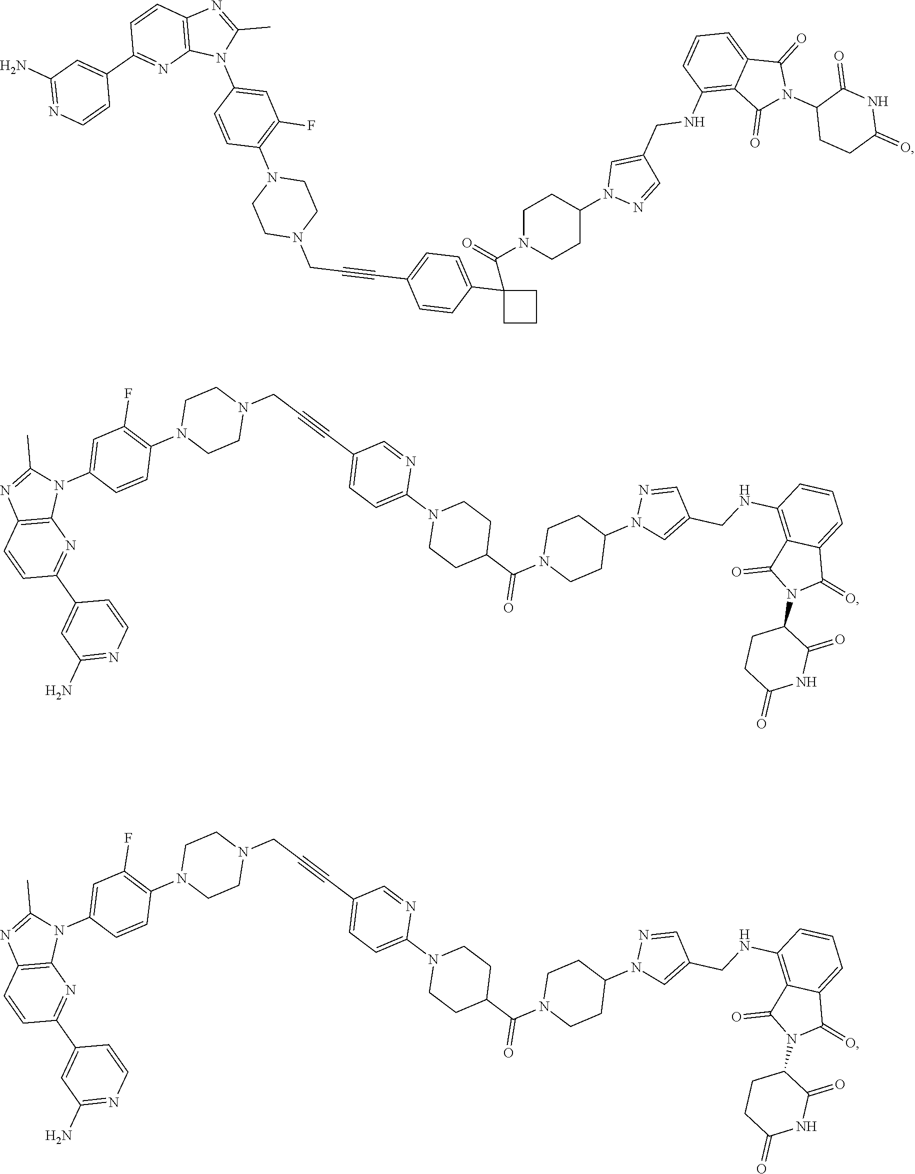 [SMILES: Cc1nc2ccc(-c3ccnc(N)c3)nc2n1-c1ccc(N2CCN(CC#Cc3ccc(C4(C(=O)N5CCC(n6cc(CNc7cccc8c7C(=O)N(C7CCC(=O)NC7=O)C8=O)cn6)CC5)CCC4)cc3)CC2)c(F)c1 ; Cc1nc2ccc(-c3ccnc(N)c3)nc2n1-c1ccc(N2CCN(CC#Cc3ccc(N4CCC(C(=O)N5CCC(n6cc(CNc7cccc8c7C(=O)N([C@@H]7CCC(=O)NC7=O)C8=O)cn6)CC5)CC4)nc3)CC2)c(F)c1 ; Cc1nc2ccc(-c3ccnc(N)c3)nc2n1-c1ccc(N2CCN(CC#Cc3ccc(N4CCC(C(=O)N5CCC(n6cc(CNc7cccc8c7C(=O)N([C@H]7CCC(=O)NC7=O)C8=O)cn6)CC5)CC4)nc3)CC2)c(F)c1]

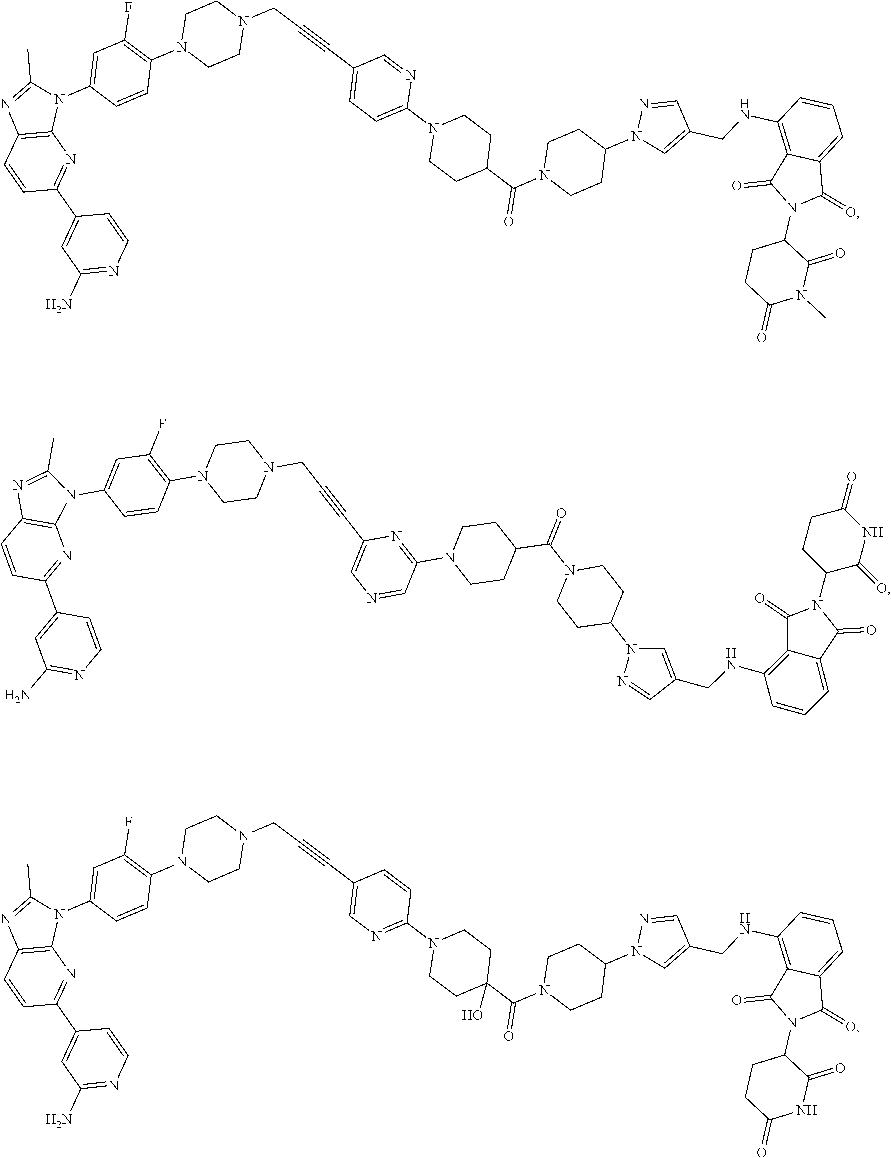 [SMILES: Cc1nc2ccc(-c3ccnc(N)c3)nc2n1-c1ccc(N2CCN(CC#Cc3ccc(N4CCC(C(=O)N5CCC(n6cc(CNc7cccc8c7C(=O)N(C7CCC(=O)N(C)C7=O)C8=O)cn6)CC5)CC4)nc3)CC2)c(F)c1 ; Cc1nc2ccc(-c3ccnc(N)c3)nc2n1-c1ccc(N2CCN(CC#Cc3cncc(N4CCC(C(=O)N5CCC(n6cc(CNc7cccc8c7C(=O)N(C7CCC(=O)NC7=O)C8=O)cn6)CC5)CC4)n3)CC2)c(F)c1 ; Cc1nc2ccc(-c3ccnc(N)c3)nc2n1-c1ccc(N2CCN(CC#Cc3ccc(N4CCC(O)(C(=O)N5CCC(n6cc(CNc7cccc8c7C(=O)N(C7CCC(=O)NC7=O)C8=O)cn6)CC5)CC4)nc3)CC2)c(F)c1]

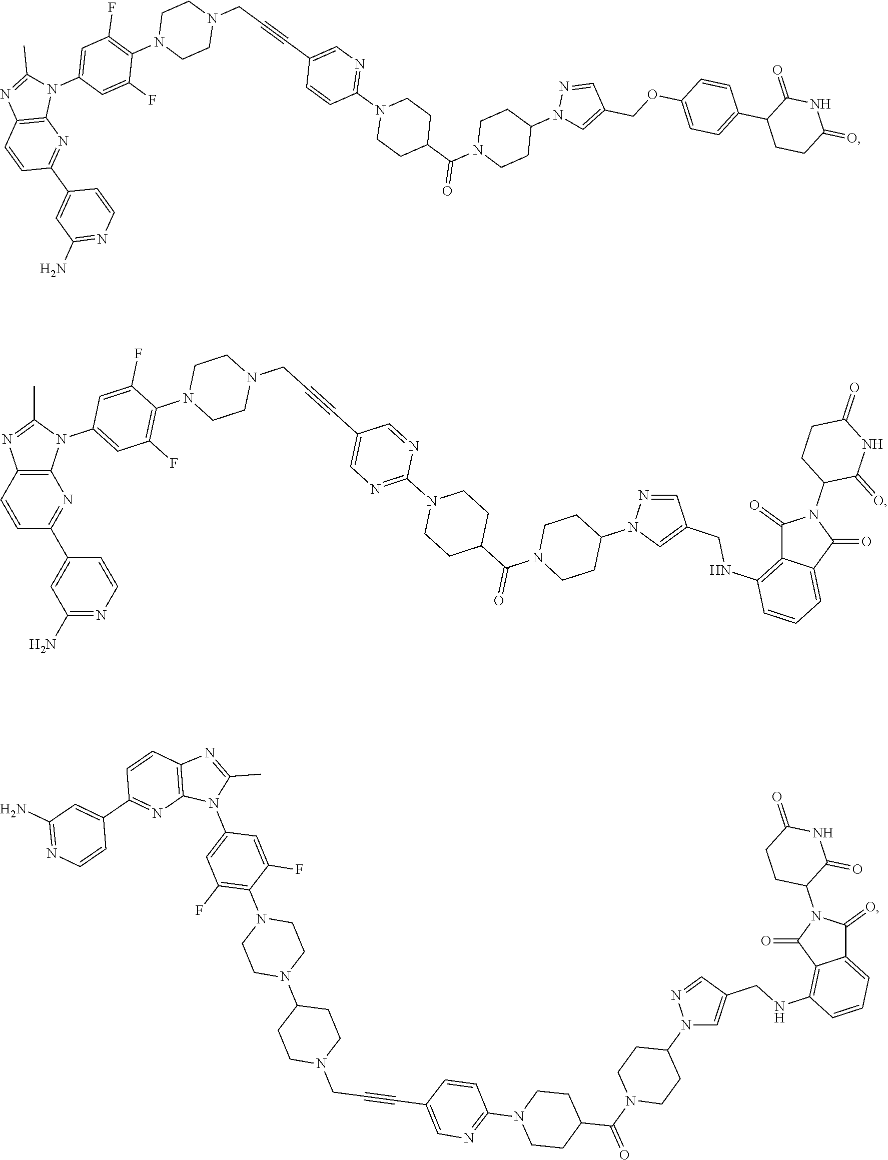 [SMILES: Cc1nc2ccc(-c3ccnc(N)c3)nc2n1-c1cc(F)c(N2CCN(CC#Cc3ccc(N4CCC(C(=O)N5CCC(n6cc(COc7ccc(C8CCC(=O)NC8=O)cc7)cn6)CC5)CC4)nc3)CC2)c(F)c1 ; Cc1nc2ccc(-c3ccnc(N)c3)nc2n1-c1cc(F)c(N2CCN(CC#Cc3cnc(N4CCC(C(=O)N5CCC(n6cc(CNc7cccc8c7C(=O)N(C7CCC(=O)NC7=O)C8=O)cn6)CC5)CC4)nc3)CC2)c(F)c1 ; Cc1nc2ccc(-c3ccnc(N)c3)nc2n1-c1cc(F)c(N2CCN(C3CCN(CC#Cc4ccc(N5CCC(C(=O)N6CCC(n7cc(CNc8cccc9c8C(=O)N(C8CCC(=O)NC8=O)C9=O)cn7)CC6)CC5)nc4)CC3)CC2)c(F)c1]

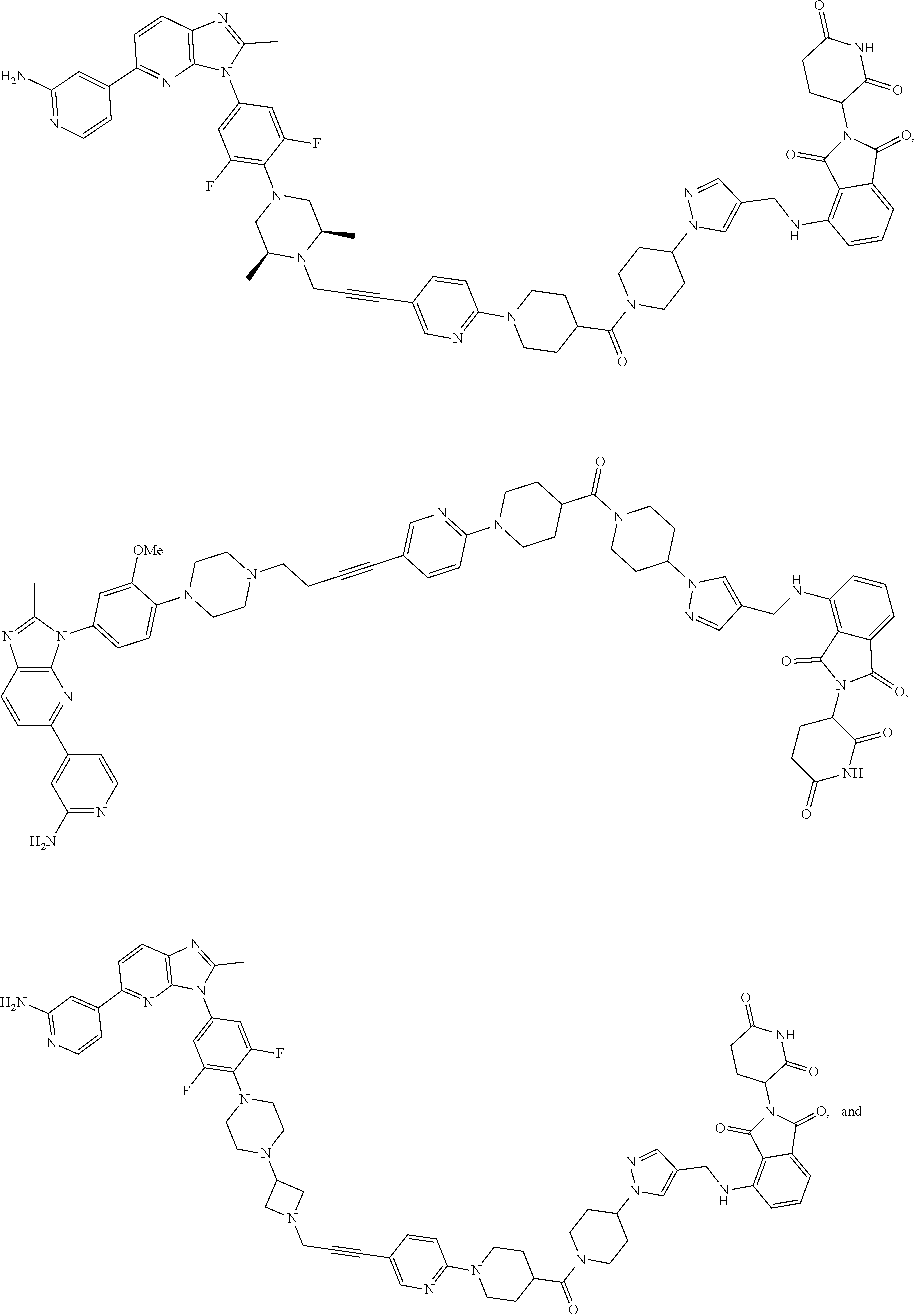 [SMILES: Cc1nc2ccc(-c3ccnc(N)c3)nc2n1-c1cc(F)c(N2C[C@@H](C)N(CC#Cc3ccc(N4CCC(C(=O)N5CCC(n6cc(CNc7cccc8c7C(=O)N(C7CCC(=O)NC7=O)C8=O)cn6)CC5)CC4)nc3)[C@@H](C)C2)c(F)c1 ; COc1cc(-n2c(C)nc3ccc(-c4ccnc(N)c4)nc32)ccc1N1CCN(CCC#Cc2ccc(N3CCC(C(=O)N4CCC(n5cc(CNc6cccc7c6C(=O)N(C6CCC(=O)NC6=O)C7=O)cn5)CC4)CC3)nc2)CC1 ; Cc1nc2ccc(-c3ccnc(N)c3)nc2n1-c1cc(F)c(N2CCN(C3CN(CC#Cc4ccc(N5CCC(C(=O)N6CCC(n7cc(CNc8cccc9c8C(=O)N(C8CCC(=O)NC8=O)C9=O)cn7)CC6)CC5)nc4)C3)CC2)c(F)c1 ; "and"]

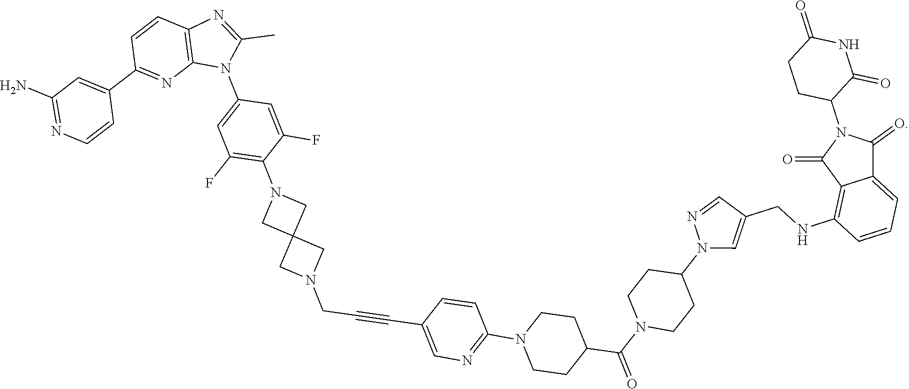 [SMILES: Cc1nc2ccc(-c3ccnc(N)c3)nc2n1-c1cc(F)c(N2CC3(CN(CC#Cc4ccc(N5CCC(C(=O)N6CCC(n7cc(CNc8cccc9c8C(=O)N(C8CCC(=O)NC8=O)C9=O)cn7)CC6)CC5)nc4)C3)C2)c(F)c1]

10. The bifunctional compound of claim 1, wherein the bifunctional compound is recited in Example I.

11. A pharmaceutical composition comprising a compound of claim 1.

12. A method of treating, ameliorating, or preventing a disorder related to one or more of DYRK1A activity, DYRK1B activity, DYRK2 activity, DYRK3 activity, CLK1 activity, CLK2 activity, CLK3 activity, CLK4 activity, and WNT signaling activity, in a patient comprising administering to said patient a therapeutically effective amount of the pharmaceutical composition of claim 11.

13. The method of claim 12, wherein said disorder is selected from Alzheimer's disease, Down syndrome, Huntington's disease, Parkinson's disease, an autoimmune disease, an inflammatory disorder (e.g., airway inflammation, osteoarthritis (e.g., knee related osteoarthritis)), diabetes, or cancer (e.g., glioblastoma, prostate cancer, metastatic breast cancer, metastatic lung cancer, multiple myeloma, secondary metastatic tumors of the brain, colorectal cancer), a viral infection (e.g., SARS-COV-2 infection (e.g., COVID-19)), and other diseases).

14. The method of claim 12, wherein said patient is a human patient.

15. The method of claim 12, further comprising administering to said patient one or more agents for treating Alzheimer's disease, Down syndrome, Huntington's disease, Parkinson's disease, autoimmune disease, an inflammatory disorder (e.g., airway inflammation, osteoarthritis (e.g., knee related osteoarthritis)), diabetes, or cancer (e.g., glioblastoma, prostate cancer, metastatic breast cancer, metastatic lung cancer, multiple myeloma, secondary metastatic tumors of the brain, colorectal cancer), a viral infection (e.g., SARS-COV-2 infection (e.g., COVID-19)), and other diseases).

16. A kit comprising a compound of claim 1 and instructions for administering said compound to a patient having a disorder related to one or more of DYRK1A activity, DYRK1B activity, DYRK2 activity, DYRK3 activity, CLK1 activity, CLK2 activity, CLK3 activity, CLK4 activity, and WNT signaling activity.

17. The kit of claim 16, wherein the disorder is Alzheimer's disease, Down syndrome, Huntington's disease, Parkinson's disease, autoimmune disease, inflammatory disorder (e.g., airway inflammation, osteoarthritis (e.g., knee related osteoarthritis)), diabetes, or cancer (e.g., glioblastoma, prostate cancer, metastatic breast cancer, metastatic lung cancer, multiple myeloma, secondary metastatic tumors of the brain, colorectal cancer), a viral infection (e.g., SARS-COV-2 infection (e.g., COVID-19)), and other diseases).

18. The kit of claim 16, further comprising one or more agents for treating Alzheimer's disease, Down syndrome, Huntington's disease, Parkinson's disease, autoimmune disease, an inflammatory disorder (e.g., airway inflammation, osteoarthritis (e.g., knee related osteoarthritis)), diabetes, or cancer (e.g., glioblastoma, prostate cancer, metastatic breast cancer, metastatic lung cancer, multiple myeloma, secondary metastatic tumors of the brain, colorectal cancer), a viral infection (e.g., SARS-COV-2 infection (e.g., COVID-19)), and other diseases).

19. A method for inhibiting one or more of DYRK1A activity, DYRK1B activity, DYRK2 activity, DYRK3 activity, CLK1 activity, CLK2 activity, CLK3 activity, CLK4 activity, and WNT signaling activity in a subject, comprising administering to said patient a therapeutically effective amount of the pharmaceutical composition of claim 11.

20. The method of claim 19, wherein administration of the compound results in inhibition of one or more of DYRK1A related PI3K/Akt signaling; DYRK1A related tau phosphorylation; DYRK1A related NFAT phosphorylation; DYRK1A related ASK1/JNK1 pathway activation; DYRK1A related p53 phosphorylation; DYRK1A related Amph 1 phosphorylation; DYRK1A related Dynamin 1 phosphorylation; DYRK1A related Synaptojanin phosphorylation; DYRK1A related presenilin 1 (the catalytic sub-unit of y-secretase) activity; DYRK1A related amyloid precursor protein phosphorylation; DYRK1A related SIRT1 activation; DYRK2 related heat shock factor 1 and 26S proteasome activities; DYRK3 related mTOR activity; DYRK3 phosphorylation (e.g., PRAS40); DYRK1B activity; CMGC/CLK kinase activity; CLK1 activity; CLK2 activity; CLK3 activity; CLK4 activity; and WNT signaling.

21. The method of claim 19, wherein the subject is human subject suffering from or at risk for developing a disorder related to DYRK1A, DYRK1B, DYRK2, DYRK3, and/or CLK1, CLK2, CLK3 and CLK4 activity.

22. The method of claim 19, wherein the disorder is Alzheimer's disease, Down syndrome, Huntington's disease, Parkinson's disease, autoimmune disease, an inflammatory disorder (e.g., airway inflammation, osteoarthritis (e.g., knee related osteoarthritis)), diabetes, or cancer (e.g., glioblastoma, prostate cancer, metastatic breast cancer, metastatic lung cancer, multiple myeloma, secondary metastatic tumors of the brain, colorectal cancer), a viral infection (e.g., SARS-COV-2 infection (e.g., COVID-19)), and other diseases).
